SEQUENCE LISTING (119 sequences)

SEQ ID NO: 1 - protein, 1255 aa, synthetic construct
  REGION 1..1255: note = Coronavirus wild-type S-protein sequence
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 2 - DNA, 3765 bp, synthetic construct
  misc_feature 1..3765: note = Coronavirus wild-type S-protein sequence
  source 1..3765: mol_type = other DNA
atgtttatctttctgctgtttctgaccctgaccagcggcagcgacctggatagatgcaccaccttcgacgatgtgcaggcccctaactacacccagcacaccagctctatgcggggcgtgtactaccccgacgagattttcagaagcgacaccctgtatctgacccaggacctgttcctgcctttctacagcaacgtgaccggcttccacaccatcaaccacaccttcggcaaccctgtgatccccttcaaggacggcatctactttgccgccaccgagaagtccaacgtcgtcagaggatgggtgttcggcagcaccatgaacaacaagagccagagcgtgatcatcatcaacaacagcaccaacgtggtcatccgggcctgcaacttcgagctgtgcgacaacccattcttcgccgtgtccaagcctatgggcacccagacacacaccatgatcttcgacaacgccttcaactgcaccttcgagtacatcagcgacgccttcagcctggacgtgtccgaaaagagcggcaacttcaagcacctgagggaattcgtgttcaagaacaaggatggcttcctgtacgtgtacaagggctaccagcctatcgacgtcgtgcgggatctgcccagcggcttcaataccctgaagcctatcttcaagctgcccctgggcatcaacatcaccaacttcagagccatcctgaccgctttcagccccgctcaggatatctggggaacaagcgccgctgcctacttcgtgggctacctgaagccaaccaccttcatgctgaagtacgacgagaacggcaccatcaccgacgccgtggactgtagccaaaatcctctggccgagctgaagtgcagcgtgaagtccttcgagatcgacaagggcatctaccagaccagcaatttcagagtggtgccctccggggatgtcgtgcggttccccaacatcacaaatctgtgccccttcggcgaggtgttcaacgccaccaagtttccctctgtgtacgcctgggagcgcaaaaagatcagcaactgcgtggccgactacagcgtgctgtacaactccaccttcttcagcaccttcaagtgctacggcgtgtccgccacaaagctgaacgacctgtgcttctccaacgtgtacgccgacagcttcgtggtcaaaggcgacgacgttcggcagattgcccctggacaaacaggcgtgatcgccgattacaactacaagctgcctgacgacttcatgggctgcgtgctggcctggaacaccagaaacatcgatgccacctccaccggcaactacaattacaagtacagatacctgcggcacggcaagctgcggcctttcgagagggatatcagcaatgtgccttttagccccgacggcaagccctgcacacctcctgctctgaattgctactggcccctgaacgactacggcttttacaccaccacaggcatcggctatcagccctatagagtggtggtcctgtcctttgagctgctgaatgcccctgccacagtgtgcggacctaagctgtctaccgacctgatcaagaaccagtgcgtgaacttcaacttcaacggcctgaccggcaccggcgtgctgacaccaagcagcaagagattccagcctttccagcagttcggccgggatgtgtccgacttcacagacagcgtcagagatcccaagaccagcgagatcctggacatcagcccttgtgcctttggcggagtgtccgtgatcacccctggcacaaatgcctctagcgaagtggccgtgctgtatcaggacgtgaactgcaccgatgtgtccaccgccattcacgccgatcagctgactcccgcttggcggatctatagcacaggcaacaacgtgttccagacacaagccggctgtctgatcggagccgagcatgtggataccagctacgagtgcgacatccctatcggcgctggcatctgtgcctcttaccacaccgtgtctctgctgcggagcaccagccagaaatccatcgtggcctacaccatgagcctgggcgccgattcttctatcgcctactccaacaacacaatcgctatccccaccaatttcagcatctccatcaccaccgaagtgatgcccgtgtccatggccaagacctccgtggattgcaacatgtacatctgcggcgacagcaccgagtgcgccaatctgctgctccagtacggcagcttctgcacccagctgaatagagccctgtctggaattgccgccgagcaggacagaaacaccagagaagtgttcgcccaagtgaagcagatgtataagaccccgacactcaagtacttcggcgggttcaacttctcccagatcctgcctgatcctctgaagcccaccaagcggagcttcatcgaggacctgctgttcaacaaagtgaccctggccgacgccggctttatgaagcagtatggcgagtgcctgggcgacatcaacgccagggatctgatttgcgcccagaagtttaacggactgaccgtgctgcctcctctgctgaccgatgatatgatcgccgcctacacagccgctctggtgtctggtacagctaccgccggatggacatttggagctggcgccgctctccagattccattcgctatgcagatggcctaccggttcaacggcatcggagtgacccagaatgtgctgtacgagaatcagaagcagatcgccaatcagttcaacaaggccatcagccagatccaagagagcctgaccaccacaagcacagccctgggaaagctccaggacgtggtcaaccagaatgctcaggccctgaacaccctggtcaagcagctgagcagcaacttcggcgccatcagctccgtgctgaatgacatcctgagccggctggacaaggtggaagcagaggtgcagatcgaccggctgatcacaggcagactccagagcctccagacctacgtgacacagcagctgatcagagccgccgagattagagcctctgccaatctggccgccaccaaaatgagcgagtgtgtcctgggccagagcaagagagtggacttttgcggcaagggctatcacctgatgagcttcccacaggccgctcctcatggcgtggtctttctgcacgtgacatacgtgcccagccaagagagaaacttcaccaccgctccagccatctgccacgagggcaaagcctactttcccagagaaggcgtgttcgtgtttaacggcacctcctggtttatcacccagcggaatttcttcagcccgcaaatcatcaccacagacaacaccttcgtgtccggcaactgtgacgtcgtgatcggcatcattaacaataccgtgtacgaccctctccagcctgagctggacagcttcaaagaggaactggataagtacttcaagaatcacacgagccccgatgtggacctgggcgatatctctggcatcaatgccagcgtcgtgaacatccagaaagagattgacaggctgaacgaggtggccaagaacctgaacgagtccctgatcgacctgcaagagctggggaagtacgagcagtacatcaagtggccttggtacgtgtggctgggctttatcgccggactgatcgccatcgtgatggtcaccatcctgctgtgctgcatgaccagctgttgcagctgtctgaagggcgcctgtagctgtggctcctgctgcaagttcgatgaggacgactctgagccagtgctgaaaggcgtgaagctgcactacacc

SEQ ID NO: 3 - protein, 1152 aa, synthetic construct
  REGION 1..1152: note = Coronavirus wild-type trS-protein sequence
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDIS

SEQ ID NO: 4 - DNA, 3456 bp, synthetic construct
  misc_feature 1..3456: note = Coronavirus wild-type tr-S-protein sequence
  source 1..3456: mol_type = other DNA
atgtttatctttctgctgtttctgaccctgaccagcggcagcgacctggatagatgcaccaccttcgacgatgtgcaggcccctaactacacccagcacaccagctctatgcggggcgtgtactaccccgacgagattttcagaagcgacaccctgtatctgacccaggacctgttcctgcctttctacagcaacgtgaccggcttccacaccatcaaccacaccttcggcaaccctgtgatccccttcaaggacggcatctactttgccgccaccgagaagtccaacgtcgtcagaggatgggtgttcggcagcaccatgaacaacaagagccagagcgtgatcatcatcaacaacagcaccaacgtggtcatccgggcctgcaacttcgagctgtgcgacaacccattcttcgccgtgtccaagcctatgggcacccagacacacaccatgatcttcgacaacgccttcaactgcaccttcgagtacatcagcgacgccttcagcctggacgtgtccgaaaagagcggcaacttcaagcacctgagggaattcgtgttcaagaacaaggatggcttcctgtacgtgtacaagggctaccagcctatcgacgtcgtgcgggatctgcccagcggcttcaataccctgaagcctatcttcaagctgcccctgggcatcaacatcaccaacttcagagccatcctgaccgctttcagccccgctcaggatatctggggaacaagcgccgctgcctacttcgtgggctacctgaagccaaccaccttcatgctgaagtacgacgagaacggcaccatcaccgacgccgtggactgtagccaaaatcctctggccgagctgaagtgcagcgtgaagtccttcgagatcgacaagggcatctaccagaccagcaatttcagagtggtgccctccggggatgtcgtgcggttccccaacatcacaaatctgtgccccttcggcgaggtgttcaacgccaccaagtttccctctgtgtacgcctgggagcgcaaaaagatcagcaactgcgtggccgactacagcgtgctgtacaactccaccttcttcagcaccttcaagtgctacggcgtgtccgccacaaagctgaacgacctgtgcttctccaacgtgtacgccgacagcttcgtggtcaaaggcgacgacgttcggcagattgcccctggacaaacaggcgtgatcgccgattacaactacaagctgcctgacgacttcatgggctgcgtgctggcctggaacaccagaaacatcgatgccacctccaccggcaactacaattacaagtacagatacctgcggcacggcaagctgcggcctttcgagagggatatcagcaatgtgccttttagccccgacggcaagccctgcacacctcctgctctgaattgctactggcccctgaacgactacggcttttacaccaccacaggcatcggctatcagccctatagagtggtggtcctgtcctttgagctgctgaatgcccctgccacagtgtgcggacctaagctgtctaccgacctgatcaagaaccagtgcgtgaacttcaacttcaacggcctgaccggcaccggcgtgctgacaccaagcagcaagagattccagcctttccagcagttcggccgggatgtgtccgacttcacagacagcgtcagagatcccaagaccagcgagatcctggacatcagcccttgtgcctttggcggagtgtccgtgatcacccctggcacaaatgcctctagcgaagtggccgtgctgtatcaggacgtgaactgcaccgatgtgtccaccgccattcacgccgatcagctgactcccgcttggcggatctatagcacaggcaacaacgtgttccagacacaagccggctgtctgatcggagccgagcatgtggataccagctacgagtgcgacatccctatcggcgctggcatctgtgcctcttaccacaccgtgtctctgctgcggagcaccagccagaaatccatcgtggcctacaccatgagcctgggcgccgattcttctatcgcctactccaacaacacaatcgctatccccaccaatttcagcatctccatcaccaccgaagtgatgcccgtgtccatggccaagacctccgtggattgcaacatgtacatctgcggcgacagcaccgagtgcgccaatctgctgctccagtacggcagcttctgcacccagctgaatagagccctgtctggaattgccgccgagcaggacagaaacaccagagaagtgttcgcccaagtgaagcagatgtataagaccccgacactcaagtacttcggcgggttcaacttctcccagatcctgcctgatcctctgaagcccaccaagcggagcttcatcgaggacctgctgttcaacaaagtgaccctggccgacgccggctttatgaagcagtatggcgagtgcctgggcgacatcaacgccagggatctgatttgcgcccagaagtttaacggactgaccgtgctgcctcctctgctgaccgatgatatgatcgccgcctacacagccgctctggtgtctggtacagctaccgccggatggacatttggagctggcgccgctctccagattccattcgctatgcagatggcctaccggttcaacggcatcggagtgacccagaatgtgctgtacgagaatcagaagcagatcgccaatcagttcaacaaggccatcagccagatccaagagagcctgaccaccacaagcacagccctgggaaagctccaggacgtggtcaaccagaatgctcaggccctgaacaccctggtcaagcagctgagcagcaacttcggcgccatcagctccgtgctgaatgacatcctgagccggctggacaaggtggaagcagaggtgcagatcgaccggctgatcacaggcagactccagagcctccagacctacgtgacacagcagctgatcagagccgccgagattagagcctctgccaatctggccgccaccaaaatgagcgagtgtgtcctgggccagagcaagagagtggacttttgcggcaagggctatcacctgatgagcttcccacaggccgctcctcatggcgtggtctttctgcacgtgacatacgtgcccagccaagagagaaacttcaccaccgctccagccatctgccacgagggcaaagcctactttcccagagaaggcgtgttcgtgtttaacggcacctcctggtttatcacccagcggaatttcttcagcccgcaaatcatcaccacagacaacaccttcgtgtccggcaactgtgacgtcgtgatcggcatcattaacaataccgtgtacgaccctctccagcctgagctggacagcttcaaagaggaactggataagtacttcaagaatcacacgagccccgatgtggacctgggcgatatctct

SEQ ID NO: 5 - protein, 213 aa, synthetic construct
  REGION 1..213: note = Coronavirus wild-type S-protein RBD sequence
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTD

SEQ ID NO: 6 - DNA, 639 bp, synthetic construct
  misc_feature 1..639: note = Coronavirus wild-type S-protein RBD sequence
  source 1..639: mol_type = other DNA
agagtggtgccctccggggatgtcgtgcggttccccaacatcacaaatctgtgccccttcggcgaggtgttcaacgccaccaagtttccctctgtgtacgcctgggagcgcaaaaagatcagcaactgcgtggccgactacagcgtgctgtacaactccaccttcttcagcaccttcaagtgctacggcgtgtccgccacaaagctgaacgacctgtgcttctccaacgtgtacgccgacagcttcgtggtcaaaggcgacgacgttcggcagattgcccctggacaaacaggcgtgatcgccgattacaactacaagctgcctgacgacttcatgggctgcgtgctggcctggaacaccagaaacatcgatgccacctccaccggcaactacaattacaagtacagatacctgcggcacggcaagctgcggcctttcgagagggatatcagcaatgtgccttttagccccgacggcaagccctgcacacctcctgctctgaattgctactggcccctgaacgactacggcttttacaccaccacaggcatcggctatcagccctatagagtggtggtcctgtcctttgagctgctgaatgcccctgccacagtgtgcggacctaagctgtctaccgac

SEQ ID NO: 7 - protein, 1273 aa, synthetic construct
  REGION 1..1273: note = Coronavirus wild-type S-protein sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - DNA, 3819 bp, synthetic construct
  misc_feature 1..3819: note = Coronavirus wild-type S-protein sequence
  source 1..3819: mol_type = other DNA
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaaaatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactacgtgggatacctccagccaagaaccttcctgctgaagtacaacgagaacggcaccatcaccgacgccgtggattgtgctctggaccctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatcagcaccgaaatctatcaggccggcagcaccccttgcaacggcgtggaaggcttcaactgctacttcccactgcaaagctacggctttcagcccacaaatggcgtgggctaccagccttacagagtggtggtgctgagcttcgagctgctgcatgctcctgccacagtgtgcggccctaagaaatccaccaatctcgtgaagaacaaatgcgtgaacttcaacttcaacggcctgaccggcaccggcgtgctgacagagagcaacaagaagttcctgccattccagcagttcggccgggatatcgccgataccacagatgccgtcagagatccccagacactggaaatcctggacatcaccccatgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgcgcctcttaccagacacagacaaacagccccagacgggctagaagcgtggccagccagagcatcattgcctacacaatgtctctgggcgccgagaacagcgtggcctactccaacaactctatcgctatccccaccaacttcaccatcagcgtgaccaccgagatcctgcctgtgtccatgaccaagaccagcgtggactgcaccatgtacatctgcggcgattccaccgagtgctccaacctgctgctccagtacggcagcttctgcacccagctgaatagagccctgacagggatcgccgtggaacaggacaagaacacccaagaggtgttcgcccaagtgaagcaaatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtacggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctccagattccattcgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctccaggacgtcgtgaaccagaatgcccaggcactgaacaccctggtcaagcagctgtcctccaacttcggcgccatcagctctgtgctgaacgatatcctgagcagactggacaaggtggaagccgaggtgcagatcgacagactgatcaccggcagactccagtctctccagacctacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgttgtgatcggcattgtgaacaataccgtgtacgaccctctccagcctgaactggactccttcaaagaggaactcgacaagtactttaagaaccacacaagccccgacgtggacctgggcgatatcagcggaatcaatgccagcgtggtcaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggctcctgctgcaagttcgacgaggacgattctgagcccgtgctgaagggcgtgaaactgcactacacc

SEQ ID NO: 9 - protein, 1170 aa, synthetic construct
  REGION 1..1170: note = Coronavirus wild-type trS-protein sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDIS

SEQ ID NO: 10 - DNA, 3510 bp, synthetic construct
  misc_feature 1..3510: note = Coronavirus wild-type trS-protein sequence
  source 1..3510: mol_type = other DNA
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaaaatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactacgtgggatacctccagccaagaaccttcctgctgaagtacaacgagaacggcaccatcaccgacgccgtggattgtgctctggaccctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatcagcaccgaaatctatcaggccggcagcaccccttgcaacggcgtggaaggcttcaactgctacttcccactgcaaagctacggctttcagcccacaaatggcgtgggctaccagccttacagagtggtggtgctgagcttcgagctgctgcatgctcctgccacagtgtgcggccctaagaaatccaccaatctcgtgaagaacaaatgcgtgaacttcaacttcaacggcctgaccggcaccggcgtgctgacagagagcaacaagaagttcctgccattccagcagttcggccgggatatcgccgataccacagatgccgtcagagatccccagacactggaaatcctggacatcaccccatgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgcgcctcttaccagacacagacaaacagccccagacgggctagaagcgtggccagccagagcatcattgcctacacaatgtctctgggcgccgagaacagcgtggcctactccaacaactctatcgctatccccaccaacttcaccatcagcgtgaccaccgagatcctgcctgtgtccatgaccaagaccagcgtggactgcaccatgtacatctgcggcgattccaccgagtgctccaacctgctgctccagtacggcagcttctgcacccagctgaatagagccctgacagggatcgccgtggaacaggacaagaacacccaagaggtgttcgcccaagtgaagcaaatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtacggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctccagattccattcgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctccaggacgtcgtgaaccagaatgcccaggcactgaacaccctggtcaagcagctgtcctccaacttcggcgccatcagctctgtgctgaacgatatcctgagcagactggacaaggtggaagccgaggtgcagatcgacagactgatcaccggcagactccagtctctccagacctacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgttgtgatcggcattgtgaacaataccgtgtacgaccctctccagcctgaactggactccttcaaagaggaactcgacaagtactttaagaaccacacaagccccgacgtggacctgggcgatatcagt

SEQ ID NO: 11 - protein, 214 aa, synthetic construct
  REGION 1..214: note = Coronavirus wild-type S-protein RBD sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 12 - DNA, 642 bp, synthetic construct
  misc_feature 1..642: note = Coronavirus S-protein RBD sequence
  source 1..642: mol_type = other DNA
cgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatcagcaccgaaatctatcaggccggcagcaccccttgcaacggcgtggaaggcttcaactgctacttcccactgcaaagctacggctttcagcccacaaatggcgtgggctaccagccttacagagtggtggtgctgagcttcgagctgctgcatgctcctgccacagtgtgcggccctaagaaatccaccaat

SEQ ID NO: 13 - protein, 1288 aa, synthetic construct
  REGION 1..1288: note = Coronavirus S-protein designed sequence
MFLFLFIIIFAFFLLSAKANERCGIFTSKPQPKLAQVSSSRRGVYYPDDIFRSDVLHLTQDYFLPFDSNVTRYFSLNANGPDRIVYFDNPIIPFKDGVYFAATEKSNVIRGWIFGSTLDNTSQSVIIVNNSTNVIIRVCNFDLCNDPFFTVSRPTDKHIKTWSIREFAVYQSAFNCTFEYVSKSFLLDVAEKPGNFKHLREFVFKNVDGFLNVYSTYKPINVVSGLPTGFSVLKPILKLPLGINITSFRVLLTMFRGDPTPGHTTANWLTAAAAYYVGYLKPTTFMLKYNENGTITDAVDCSQNPLAELKCTLKNFNVDKGIYQTSNFRVSPTQEVVRFPNITNLCPFDKVFNATRFPSVYAWERTKISDCVADYTVLYNSTSFSTFKCYGVSPSKLIDLCFTSVYADTFLIRCSEVRQVAPGQTGVIADYNYKLPDDFTGCVIAWNTAKQDTGSSGNYNYYYRSHRKTKLKPFERDLSSDECSPDGKPCTPPAFNGVRGFNCYFTLSTYDFNPNVPVEYQATRVVVLSFELLNAPATVCGPKLSTQLVKNQCVNFNFNGLKGTGVLTASSKRFQSFQQFGRDASDFTDSVRDPQTLEILDISPCSFGGVSVITPGTNTSSEVAVLYQDVNCTDVPTAIHADQLTPAWRVYSTGVNVFQTQAGCLIGAEHVNASYECDIPIGAGICASYHTASNSPRILRSTGQKSIVAYTMSLGAENSIAYANNSIAIPTNFSISVTTEVMPVSMAKTSVDCTMYICGDSLECSNLLLQYGSFCTQLNRALTGIAIEQDKNTQEVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMATILLCCMTSCCSCLKGACSCGSCCKFDEDDSEPVLKGVKLHYT

SEQ ID NO: 14 - DNA, 3864 bp, synthetic construct
  misc_feature 1..3864: note = Coronavirus S-protein designed sequence
  source 1..3864: mol_type = other DNA
atgtttctgttcctcttcattattatcttcgcattcttcctgctgagcgccaaggccaacgagagatgcggcatcttcaccagcaagccccagcctaagctggcccaggtgtccagttctagacggggcgtgtactaccccgacgacatcttcagatccgacgtgctgcatctgacccaggactacttcctgcctttcgacagcaacgtgacccggtacttcagcctgaacgccaacggacccgaccggatcgtgtacttcgacaaccctatcatccccttcaaggacggggtgtactttgccgccaccgagaagtccaacgtgatcagaggctggatcttcggcagcaccctggacaataccagccagagcgtgatcatcgtgaacaacagcaccaacgtcatcatccgcgtgtgcaacttcgacctgtgcaacgacccattcttcaccgtgtccagaccaaccgacaagcacatcaagacctggtccatccgcgagttcgccgtgtaccagagcgccttcaattgcaccttcgagtacgtgtccaagagctttctgctggacgtggccgagaagcccggcaactttaagcacctgagagaattcgtgttcaagaacgtggacggcttcctgaacgtgtacagcacctacaagcccatcaacgtggtgtccggcctgcctacaggattcagcgtgctgaagcccatcctgaagctgcccctgggcatcaacatcaccagcttcagagtgctgctgaccatgttcagaggcgaccctacacctggccacaccaccgctaattggctgacagccgccgctgcctactacgtgggatacctgaagcctaccaccttcatgctcaagtacaacgagaacggcaccatcaccgacgccgtggactgtagccaaaatcctctggccgagctgaagtgcaccctgaagaacttcaacgtggacaagggcatctaccagaccagcaacttccgggtgtcccctacacaagaggtcgtgcggttccccaatatcaccaatctgtgccccttcgacaaggtgttcaacgccaccagatttcccagcgtgtacgcctgggagcgcaccaagatttccgattgcgtggccgactacaccgtgctgtataactccacctccttcagcaccttcaagtgctacggcgtgtccccaagcaagctgatcgatctgtgcttcacctctgtgtacgccgacaccttcctgatccggtgtagcgaagtgcgacaggtggcacctggacagacaggcgtgatcgccgattacaactacaagctgcccgacgacttcaccggctgtgtgatcgcctggaataccgccaagcaggatacaggcagcagcggcaactacaactactactacagaagccaccgcaagaccaagctgaagcctttcgagagggacctgagcagcgacgagtgtagccctgatggcaagccttgtacacctcctgccttcaatggcgtgcggggcttcaactgctacttcaccctgagcacctacgacttcaaccccaacgtgcccgtggaataccaggccacaagagtggtggtgctgagcttcgagctgctgaatgcccctgccacagtgtgtggccctaagctgtctacccagctggtcaagaaccagtgcgtgaacttcaatttcaacggcctgaaaggcaccggcgtgctgaccgccagcagcaagagattccagagcttccagcagttcggcagggacgccagcgatttcacagatagcgtcagagatccccagacactggaaatcctggacatcagcccttgcagcttcggcggagtgtctgtgatcacccctggcaccaatacctctagcgaggtggcagtgctgtaccaggacgtgaactgcaccgatgtgcctacagccatccacgccgatcagctgacaccagcttggagagtgtactctaccggtgtcaacgtgttccagacacaagccggctgtctgattggagccgaacacgtgaacgccagctacgagtgcgacatccctatcggagccggcatctgtgcctcttaccacaccgcctctaacagccccagaatcctgagaagcaccggccagaaatccatcgtggcctacacaatgtctctgggcgccgagaactctatcgcctacgccaacaactccattgctatccccaccaacttcagcatctccgtgaccaccgaagtgatgcctgtgtccatggccaagaccagcgtggactgcacaatgtacatctgcggcgacagcctggaatgcagcaacctgctgctccagtacggcagcttctgcacccagctgaatagagccctgaccggaatcgccatcgagcaggacaagaacacccaagaggtgttcgcccaagtgaagcagatgtataagacccctgccatcaaggacttcggcggctttaacttcagccagatcctgcctgatcctagcaagcccaccaagcggagcttcatcgaggacctgctgttcaacaaagtgaccctggccgacgccggctttatgaagcagtatggcgagtgcctgggcgacatctctgccagggatctgatttgcgcccagaagttcaacggactgaccgtgctgcctcctctgctgaccgatgagatgatcgccgcctatacagccgctctggtgtctggcacagctaccgccggatggacatttggagctggcgccgctctccagattccattcgctatgcagatggcctaccgcttcaacggcatcggcgtgacccagaacgtgctgtacgagaaccagaagcagatcgccaaccagttcaacaaggccatcagtcagatccaagagagcctgaccacaaccagcacagccctgggaaagctccaggacgtcgtgaaccagaatgcccaggctctgaacaccctggtcaagcagctgagcagcaatttcggcgccatcagctccgtgctgaacgacatcctgagccggctggataaggtggaagccgaggtgcagatcgaccggctgattacaggcagactccagtctctccagacctacgtgacacagcagctgatcagagccgccgagattagagcctctgccaatctggccgccaccaagatgtctgagtgtgtgctgggccagtctaagagagtggacttctgcggcaagggctaccacctgatgagcttccctcaggctgctcctcacggcgtggtgtttctgcacgtgacatacgtgcccagccaagagcggaacttcacaactgccccagccatctgccacgagggcaaagcctactttcccagagaaggcgtgttcgtgtccaacggcacctcctggttcatcacccagagaaacttctacagccctcagatcatcaccaccgacaacaccttcgtgtccggcaactgcgacgtggtcatcggcatcatcaacaataccgtgtacgaccctctccagccagaactggatagcttcaaagaggaactcgacaagtacttcaagaatcacacaagccccgacgtggacctgggcgatatcagcggaatcaatgccagcgtggtcaacatccagaaagagatcgacagactgaacgaggtggccaagaacctgaacgagtccctgatcgacctgcaagagctggggaagtacgagcagtacatcaagtggccttggtacgtgtggctgggctttatcgccggactgatcgccattgtgatggccaccatcctgctgtgctgcatgacaagctgctgtagctgcctgaagggcgcctgtagctgtggcagctgctgcaagttcgacgaggacgattctgagcctgtgctgaaaggcgtgaagctgcactacacc

SEQ ID NO: 15 - protein, 1185 aa, synthetic construct
  REGION 1..1185: note = Coronavirus trS-protein designed sequence
MFLFLFIIIFAFFLLSAKANERCGIFTSKPQPKLAQVSSSRRGVYYPDDIFRSDVLHLTQDYFLPFDSNVTRYFSLNANGPDRIVYFDNPIIPFKDGVYFAATEKSNVIRGWIFGSTLDNTSQSVIIVNNSTNVIIRVCNFDLCNDPFFTVSRPTDKHIKTWSIREFAVYQSAFNCTFEYVSKSFLLDVAEKPGNFKHLREFVFKNVDGFLNVYSTYKPINVVSGLPTGFSVLKPILKLPLGINITSFRVLLTMFRGDPTPGHTTANWLTAAAAYYVGYLKPTTFMLKYNENGTITDAVDCSQNPLAELKCTLKNFNVDKGIYQTSNFRVSPTQEVVRFPNITNLCPFDKVFNATRFPSVYAWERTKISDCVADYTVLYNSTSFSTFKCYGVSPSKLIDLCFTSVYADTFLIRCSEVRQVAPGQTGVIADYNYKLPDDFTGCVIAWNTAKQDTGSSGNYNYYYRSHRKTKLKPFERDLSSDECSPDGKPCTPPAFNGVRGFNCYFTLSTYDFNPNVPVEYQATRVVVLSFELLNAPATVCGPKLSTQLVKNQCVNFNFNGLKGTGVLTASSKRFQSFQQFGRDASDFTDSVRDPQTLEILDISPCSFGGVSVITPGTNTSSEVAVLYQDVNCTDVPTAIHADQLTPAWRVYSTGVNVFQTQAGCLIGAEHVNASYECDIPIGAGICASYHTASNSPRILRSTGQKSIVAYTMSLGAENSIAYANNSIAIPTNFSISVTTEVMPVSMAKTSVDCTMYICGDSLECSNLLLQYGSFCTQLNRALTGIAIEQDKNTQEVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVSGNCDVVIGIINNTVYDPLQPELDSFKEELDKYFKNHTSPDVDLGDIS

SEQ ID NO: 16 - DNA, 3555 bp, synthetic construct
  misc_feature 1..3555: note = Coronavirus trS-protein designed sequence
  source 1..3555: mol_type = other DNA
atgtttctgttcctcttcattattatcttcgcattcttcctgctgagcgccaaggccaacgagagatgcggcatcttcaccagcaagccccagcctaagctggcccaggtgtccagttctagacggggcgtgtactaccccgacgacatcttcagatccgacgtgctgcatctgacccaggactacttcctgcctttcgacagcaacgtgacccggtacttcagcctgaacgccaacggacccgaccggatcgtgtacttcgacaaccctatcatccccttcaaggacggggtgtactttgccgccaccgagaagtccaacgtgatcagaggctggatcttcggcagcaccctggacaataccagccagagcgtgatcatcgtgaacaacagcaccaacgtcatcatccgcgtgtgcaacttcgacctgtgcaacgacccattcttcaccgtgtccagaccaaccgacaagcacatcaagacctggtccatccgcgagttcgccgtgtaccagagcgccttcaattgcaccttcgagtacgtgtccaagagctttctgctggacgtggccgagaagcccggcaactttaagcacctgagagaattcgtgttcaagaacgtggacggcttcctgaacgtgtacagcacctacaagcccatcaacgtggtgtccggcctgcctacaggattcagcgtgctgaagcccatcctgaagctgcccctgggcatcaacatcaccagcttcagagtgctgctgaccatgttcagaggcgaccctacacctggccacaccaccgctaattggctgacagccgccgctgcctactacgtgggatacctgaagcctaccaccttcatgctcaagtacaacgagaacggcaccatcaccgacgccgtggactgtagccaaaatcctctggccgagctgaagtgcaccctgaagaacttcaacgtggacaagggcatctaccagaccagcaacttccgggtgtcccctacacaagaggtcgtgcggttccccaatatcaccaatctgtgccccttcgacaaggtgttcaacgccaccagatttcccagcgtgtacgcctgggagcgcaccaagatttccgattgcgtggccgactacaccgtgctgtataactccacctccttcagcaccttcaagtgctacggcgtgtccccaagcaagctgatcgatctgtgcttcacctctgtgtacgccgacaccttcctgatccggtgtagcgaagtgcgacaggtggcacctggacagacaggcgtgatcgccgattacaactacaagctgcccgacgacttcaccggctgtgtgatcgcctggaataccgccaagcaggatacaggcagcagcggcaactacaactactactacagaagccaccgcaagaccaagctgaagcctttcgagagggacctgagcagcgacgagtgtagccctgatggcaagccttgtacacctcctgccttcaatggcgtgcggggcttcaactgctacttcaccctgagcacctacgacttcaaccccaacgtgcccgtggaataccaggccacaagagtggtggtgctgagcttcgagctgctgaatgcccctgccacagtgtgtggccctaagctgtctacccagctggtcaagaaccagtgcgtgaacttcaatttcaacggcctgaaaggcaccggcgtgctgaccgccagcagcaagagattccagagcttccagcagttcggcagggacgccagcgatttcacagatagcgtcagagatccccagacactggaaatcctggacatcagcccttgcagcttcggcggagtgtctgtgatcacccctggcaccaatacctctagcgaggtggcagtgctgtaccaggacgtgaactgcaccgatgtgcctacagccatccacgccgatcagctgacaccagcttggagagtgtactctaccggtgtcaacgtgttccagacacaagccggctgtctgattggagccgaacacgtgaacgccagctacgagtgcgacatccctatcggagccggcatctgtgcctcttaccacaccgcctctaacagccccagaatcctgagaagcaccggccagaaatccatcgtggcctacacaatgtctctgggcgccgagaactctatcgcctacgccaacaactccattgctatccccaccaacttcagcatctccgtgaccaccgaagtgatgcctgtgtccatggccaagaccagcgtggactgcacaatgtacatctgcggcgacagcctggaatgcagcaacctgctgctccagtacggcagcttctgcacccagctgaatagagccctgaccggaatcgccatcgagcaggacaagaacacccaagaggtgttcgcccaagtgaagcagatgtataagacccctgccatcaaggacttcggcggctttaacttcagccagatcctgcctgatcctagcaagcccaccaagcggagcttcatcgaggacctgctgttcaacaaagtgaccctggccgacgccggctttatgaagcagtatggcgagtgcctgggcgacatctctgccagggatctgatttgcgcccagaagttcaacggactgaccgtgctgcctcctctgctgaccgatgagatgatcgccgcctatacagccgctctggtgtctggcacagctaccgccggatggacatttggagctggcgccgctctccagattccattcgctatgcagatggcctaccgcttcaacggcatcggcgtgacccagaacgtgctgtacgagaaccagaagcagatcgccaaccagttcaacaaggccatcagtcagatccaagagagcctgaccacaaccagcacagccctgggaaagctccaggacgtcgtgaaccagaatgcccaggctctgaacaccctggtcaagcagctgagcagcaatttcggcgccatcagctccgtgctgaacgacatcctgagccggctggataaggtggaagccgaggtgcagatcgaccggctgattacaggcagactccagtctctccagacctacgtgacacagcagctgatcagagccgccgagattagagcctctgccaatctggccgccaccaagatgtctgagtgtgtgctgggccagtctaagagagtggacttctgcggcaagggctaccacctgatgagcttccctcaggctgctcctcacggcgtggtgtttctgcacgtgacatacgtgcccagccaagagcggaacttcacaactgccccagccatctgccacgagggcaaagcctactttcccagagaaggcgtgttcgtgtccaacggcacctcctggttcatcacccagagaaacttctacagccctcagatcatcaccaccgacaacaccttcgtgtccggcaactgcgacgtggtcatcggcatcatcaacaataccgtgtacgaccctctccagccagaactggatagcttcaaagaggaactcgacaagtacttcaagaatcacacaagccccgacgtggacctgggcgatatcagt

SEQ ID NO: 17 - protein, 219 aa, synthetic construct
  REGION 1..219: note = Coronavirus S-protein RBD designed sequence
RVSPTQEVVRFPNITNLCPFDKVFNATRFPSVYAWERTKISDCVADYTVLYNSTSFSTFKCYGVSPSKLIDLCFTSVYADTFLIRCSEVRQVAPGQTGVIADYNYKLPDDFTGCVIAWNTAKQDTGSSGNYNYYYRSHRKTKLKPFERDLSSDECSPDGKPCTPPAFNGVRGFNCYFTLSTYDFNPNVPVEYQATRVVVLSFELLNAPATVCGPKLSTQ

SEQ ID NO: 18 - DNA, 657 bp, synthetic construct
  misc_feature 1..657: note = Coronavirus S-protein RBD designed sequence
  source 1..657: mol_type = other DNA
cgggtgtcccctacacaagaggtcgtgcggttccccaatatcaccaatctgtgccccttcgacaaggtgttcaacgccaccagatttcccagcgtgtacgcctgggagcgcaccaagatttccgattgcgtggccgactacaccgtgctgtataactccacctccttcagcaccttcaagtgctacggcgtgtccccaagcaagctgatcgatctgtgcttcacctctgtgtacgccgacaccttcctgatccggtgtagcgaagtgcgacaggtggcacctggacagacaggcgtgatcgccgattacaactacaagctgcccgacgacttcaccggctgtgtgatcgcctggaataccgccaagcaggatacaggcagcagcggcaactacaactactactacagaagccaccgcaagaccaagctgaagcctttcgagagggacctgagcagcgacgagtgtagccctgatggcaagccttgtacacctcctgccttcaatggcgtgcggggcttcaactgctacttcaccctgagcacctacgacttcaaccccaacgtgcccgtggaataccaggccacaagagtggtggtgctgagcttcgagctgctgaatgcccctgccacagtgtgtggccctaagctgtctacccag

SEQ ID NO: 19 - DNA, 91 bp, synthetic construct
  misc_feature 1..91: note = pEVAC Multiple Cloning Site sequence
  source 1..91: mol_type = other DNA
acagactgttcctttccatgggtcttttctgcagtcaccgtcggtaccgtcgacacgtgtgatcatctagaggatccgcggccgcagatct

SEQ ID NO: 20 - DNA, 4405 bp, synthetic construct
  misc_feature 1..4405: note = pEVAC sequence
  source 1..4405: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcggtaccgtcgacacgtgtgatcatctagaggatccgcggccgcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 21 - protein, 76 aa, synthetic construct
  REGION 1..76: note = SARS envelope protein sequence
MYSFVSEETGTLIVNSVLLFLAFVVFLLVTLAILTALRLCAYCCNIVNVSLVKPTVYVYSRVKNLNSSEGVPDLLV

SEQ ID NO: 22 - protein, 76 aa, synthetic construct
  REGION 1..76: note = Coronavirus envelope protein designed sequence
MYSFVSEETGTLIVNSVLLFLAFVVFLLVTLAILTALRLCAYCCNIVNVSLVKPTFYVYSRVKNLNSSQGVPDLLV

SEQ ID NO: 23 - protein, 75 aa, synthetic construct
  REGION 1..75: note = Coronavirus envelope protein designed sequence
MYSFVSEETGTLIVNSVLLFLAFVVFLLVTLAILTALRLCAYCCNIVNVSLVKPTFYVYSRVKNLNSSRVPDLLV

SEQ ID NO: 24 - protein, 221 aa, synthetic construct
  REGION 1..221: note = Coronavirus M protein designed sequence
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTILTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 25 - protein, 222 aa, synthetic construct
  REGION 1..222: note = Coronavirus M protein designed sequence
MADSNGTITVEELKKLLEQWNLVIGFLFLTWICLLQFAYSNRNRFLYIIKLIFLWLLWPVTLACFVLAAVYRINWVTGGIAIAMACIVGLMWLSYFVASFRLFARTRSMWSFNPETNILLNVPLRGSIITRPLMESELVIGAVILRGHLRMAGHSLGRCDIKDLPKEITVATSRTLSYYKLGASQRVASDSGFAVYNRYRIGNYKLNTDHSSSSDNIALLVQ

SEQ ID NO: 26 - protein, 222 aa, synthetic construct
  REGION 1..222: note = Coronavirus M protein reference sequence
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 27 - protein, 214 aa, synthetic construct
  REGION 1..214: note = Coronavirus S-protein RBD designed sequence
RVAPTKEVVRFPNITNLCPFGEVFNATRFPSVYAWERKRISNCVADYSVLYNSTSFSTFKCYGVSPTKLNDLCFTNVYADSFVIRGDEVRQIAPGQTGVIADYNYKLPDDFTGCVIAWNTNNLDSTTGGNYNYLYRSLRKSKLKPFERDISSDIYSPGGKPCSGVEGFNCYYPLRSYGFFPTNGVGYQPYRVVVLSFELLNAPATVCGPKLSTD

SEQ ID NO: 28 - protein, 214 aa, synthetic construct
  REGION 1..214: note = Coronavirus S-protein RBD designed sequence
RVAPTKEVVRFPNITNLCPFGEVFNATKFPSVYAWERKKISNCVADYSVLYNSTSFSTFKCYGVSPTKLNDLCFTNVYADSFVIRGDEVRQIAPGQTGVIADYNYKLPDDFTGCVIAWNTNNIDSTTGGNYNYLYRSLRKSKLKPFERDISSDIYSPGGKPCSGVEGFNCYYPLRSYGFFPTNGVGYQPYRVVVLSFELLNAPATVCGPKLSTD

SEQ ID NO: 29 - protein, 214 aa, synthetic construct
  REGION 1..214: note = Coronavirus S-protein RBD designed sequence
RVAPTKEVVRFPNITNLCPFGEVFNATRFPSVYAWERKRISNCVADYSVLYNSTFFSTFKCYGVSPTKLNDLCFSNVYADSFVIRGDEVRQIAPGQTGVIADYNYKLPDDFMGCVIAWNTNNLDSTTGGNYNYLYRSLRKSKLKPFERDISSDIYSPGGKPCSGVEGFNCYYPLRSYGFFPTNGVGYQPYRVVVLSFELLNAPATVCGPKLSTD

SEQ ID NO: 30 - protein, 214 aa, synthetic construct
  REGION 1..214: note = Coronavirus S-protein RBD designed sequence
RVAPTKEVVRFPNITNLCPFGEVFNATRFPSVYAWERKRISNCVADYSVLYNSTSFSTFKCYGVSPTKLNDLCFTNVYADSFVIRGDEVRQIAPGQTGKIADYNYKLPDDFTGCVIAWNTNNLDSTTGGNYNYLYRLFRKSNLKPFERDISSDIYQAGSTPCSGVEGFNCYFPLQSYGFQPTNGVGYQPYRVVVLSFELLNAPATVCGPKLSTD

SEQ ID NO: 31 - protein, 214 aa, synthetic construct
  REGION 1..214: note = Coronavirus S-protein RBD designed sequence
RVAPTKEVVRFPNITNLCPFGEVFNATKFPSVYAWERKKISNCVADYSVLYNSTSFSTFKCYGVSPTKLNDLCFTNVYADSFVIRGDEVRQIAPGQTGVIADYNYKLPDDFTGCVIAWNTNNIDSTTGGNYNYLYRSLRKSKLKPFERDISSDIYSPGGKPCSGVEGFNCYYPLRSYGFFPTNGTGYQPYRVVVLSFELLNAPATVCGPKLSTD

SEQ ID NO: 32 - protein, 214 aa, synthetic construct
  REGION 1..214: note = Coronavirus S-protein RBD designed sequence
RVAPTKEVVRFPNITNLCPFGEVFNATRFPSVYAWERKRISNCVADYSVLYNSTFFSTFKCYGVSPTKLNDLCFSNVYADSFVIRGDEVRQIAPGQTGVIADYNYKLPDDFMGCVIAWNTNNLDSTTGGNYNYLYRSLRKSKLKPFERDISSDIYSPGGKPCSGVEGFNCYYPLRSYGFFPTNGTGYQPYRVVVLSFELLNAPATVCGPKLSTD

SEQ ID NO: 33 - protein, 214 aa, synthetic construct
  REGION 1..214: note = Coronavirus S-protein RBD designed sequence
  SITE 203: note = MISC_FEATURE - Glycosylation site introduced at this residue
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHANATVCGPKKSTN

SEQ ID NO: 34 - protein, 214 aa, synthetic construct
  REGION 1..214: note = Coronavirus S-protein RBD designed sequence
  SITE 13: note = MISC_FEATURE - Glycosylation site deleted at this residue
  SITE 25: note = MISC_FEATURE - Glycosylation site deleted at this residue
  SITE 54: note = MISC_FEATURE - Glycosylation site introduced at this residue
RVQPTESIVRFPQITNLCPFGEVFQATRFASVYAWNRKRISNCVADYSVLYNSTSFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 35 - protein, 219 aa, synthetic construct
  REGION 1..219: note = Coronavirus S-protein RBD designed sequence
  SITE 203: note = MISC_FEATURE - Glycosylation site introduced at this residue
RVSPTQEVVRFPNITNLCPFDKVFNATRFPSVYAWERTKISDCVADYTVLYNSTSFSTFKCYGVSPSKLIDLCFTSVYADTFLIRCSEVRQVAPGQTGVIADYNYKLPDDFTGCVIAWNTAKQDTGSSGNYNYYYRSHRKTKLKPFERDLSSDECSPDGKPCTPPAFNGVRGFNCYFTLSTYDFNPNVPVEYQATRVVVLSFELLNANATVCGPKLSTQ

SEQ ID NO: 36 - protein, 219 aa, synthetic construct
  REGION 1..219: note = Coronavirus S-protein RBD designed sequence
  SITE 13: note = MISC_FEATURE - Glycosylation site deleted at this residue
  SITE 25: note = MISC_FEATURE - Glycosylation site deleted at this residue
RVSPTQEVVRFPQITNLCPFDKVFQATRFPSVYAWERTKISDCVADYTVLYNSTSFSTFKCYGVSPSKLIDLCFTSVYADTFLIRCSEVRQVAPGQTGVIADYNYKLPDDFTGCVIAWNTAKQDTGSSGNYNYYYRSHRKTKLKPFERDLSSDECSPDGKPCTPPAFNGVRGFNCYFTLSTYDFNPNVPVEYQATRVVVLSFELLNAPATVCGPKLSTQ

SEQ ID NO: 37 - DNA, 642 bp, synthetic construct
  misc_feature 1..642: note = Coronavirus S-protein RBD designed sequence
  source 1..642: mol_type = other DNA
cgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatcagcaccgaaatctatcaggccggcagcaccccttgcaacggcgtggaaggcttcaactgctacttcccactgcaaagctacggctttcagcccacaaatggcgtgggctaccagccttacagagtggtggtgctgagcttcgagctgctgcatgctaacgccacagtgtgcggccctaagaaatccaccaat

SEQ ID NO: 38 - DNA, 642 bp, synthetic construct
  misc_feature 1..642: note = Coronavirus S-protein RBD designed sequence
  source 1..642: mol_type = other DNA
cgggtgcagcccaccgaatccatcgtgcggttcccccagatcaccaatctgtgccccttcggcgaggtgttccaggccaccagattcgcctctgtgtacgcctggaaccggaagcggatcagcaattgcgtggccgactactccgtgctgtacaactccaccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatcagcaccgaaatctatcaggccggcagcaccccttgcaacggcgtggaaggcttcaactgctacttcccactgcaaagctacggctttcagcccacaaatggcgtgggctaccagccttacagagtggtggtgctgagcttcgagctgctgcatgctcctgccacagtgtgcggccctaagaaatccaccaat

SEQ ID NO: 39 - DNA, 657 bp, synthetic construct
  misc_feature 1..657: note = Coronavirus S-protein RBD designed sequence
  source 1..657: mol_type = other DNA
cgggtgtcccctacacaagaggtcgtgcggttccccaatatcaccaatctgtgccccttcgacaaggtgttcaacgccaccagatttcccagcgtgtacgcctgggagcgcaccaagatttccgattgcgtggccgactacaccgtgctgtataactccacctccttcagcaccttcaagtgctacggcgtgtccccaagcaagctgatcgatctgtgcttcacctctgtgtacgccgacaccttcctgatccggtgtagcgaagtgcgacaggtggcacctggacagacaggcgtgatcgccgattacaactacaagctgcccgacgacttcaccggctgtgtgatcgcctggaataccgccaagcaggatacaggcagcagcggcaactacaactactactacagaagccaccgcaagaccaagctgaagcctttcgagagggacctgagcagcgacgagtgtagccctgatggcaagccttgtacacctcctgccttcaatggcgtgcggggcttcaactgctacttcaccctgagcacctacgacttcaaccccaacgtgcccgtggaataccaggccacaagagtggtggtgctgagcttcgagctgctgaatgccaacgccacagtgtgtggccctaagctgtctacccag

SEQ ID NO: 40 - DNA, 657 bp, synthetic construct
  misc_feature 1..657: note = Coronavirus S-protein RBD designed sequence
  source 1..657: mol_type = other DNA
cgggtgtcccctacacaagaggtcgtgcggttcccccagatcaccaatctgtgccccttcgacaaggtgttccaggccaccagatttcccagcgtgtacgcctgggagcgcaccaagatttccgattgcgtggccgactacaccgtgctgtataactccacctccttcagcaccttcaagtgctacggcgtgtccccaagcaagctgatcgatctgtgcttcacctctgtgtacgccgacaccttcctgatccggtgtagcgaagtgcgacaggtggcacctggacagacaggcgtgatcgccgattacaactacaagctgcccgacgacttcaccggctgtgtgatcgcctggaataccgccaagcaggatacaggcagcagcggcaactacaactactactacagaagccaccgcaagaccaagctgaagcctttcgagagggacctgagcagcgacgagtgtagccctgatggcaagccttgtacacctcctgccttcaatggcgtgcggggcttcaactgctacttcaccctgagcacctacgacttcaaccccaacgtgcccgtggaataccaggccacaagagtggtggtgctgagcttcgagctgctgaatgcccctgccacagtgtgtggccctaagctgtctacccag

SEQ ID NO: 41 - protein, 75 aa, synthetic construct
  REGION 1..75: note = SARS2 envelope protein reference sequence
MYSFVSEETGTLIVNSVLLFLAFVVFLLVTLAILTALRLCAYCCNIVNVSLVKPSFYVYSRVKNLNSSRVPDLLV

SEQ ID NO: 42 - protein, 75 aa, synthetic construct
  REGION 1..75: note = Coronavirus envelope protein designed sequence
MYSFVSEETGTLIVASVLLFLAFVVFLLVTLAILTALRLCAYCCNIVNVSLVKPSFYVYSRVKNLNSSRVPDLLV

SEQ ID NO: 43 - protein, 76 aa, synthetic construct
  REGION 1..76: note = Coronavirus envelope protein designed sequence
MYSFVSEETGTLIVASVLLFLAFVVFLLVTLAILTALRLCAYCCNIVNVSLVKPTFYVYSRVKNLNSSQGVPDLLV

SEQ ID NO: 44 - protein, 75 aa, synthetic construct
  REGION 1..75: note = Coronavirus envelope protein designed sequence
MYSFVSEETGTLIVASVLLFLAFVVFLLVTLAILTALRLCAYCCNIVNVSLVKPTFYVYSRVKNLNSSRVPDLLV

SEQ ID NO: 45 - protein, 419 aa, synthetic construct
  REGION 1..419: note = Coronavirus nucleoprotein reference sequence
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 46 - protein, 418 aa, synthetic construct
  REGION 1..418: note = Coronavirus nucleoprotein designed sequence
MSDNGPQNQRSAPRITFGGPSDSTDNNQNGERSGARPKQRRPQGLPNNTASWFTALTQHGKEDLRFPRGQGVPINTNSGKDDQIGYYRRATRRVRGGDGKMKELSPRWYFYYLGTGPEAALPYGANKEGIVWVATEGALNTPKDHIGTRNPNNNAAIVLQLPQGTTLPKGFYAEGSRGGSQASSRSSSRSRGNSRNSTPGSSRGTSPARMASGGGDTALALLLLDRLNQLESKVSGKGQQQQGQTVTKKSAAEASKKPRQKRTATKQYNVTQAFGRRGPEQTQGNFGDQELIRQGTDYKHWPQIAQFAPSASAFFGMSREVTPSGTWLTYHGAIKLDDKDPQFKDNVILLNKHIDAYKTFPPTEPKKDKKKKADEAQPLPQRQKKQPTVTLLPAADLDDFSKQLQNSMSGASADSTQA

SEQ ID NO: 47 - protein, 417 aa, synthetic construct
  REGION 1..417: note = Coronavirus nucleoprotein designed sequence
MTDNGQQGPRNAPRITFGVSDNFDNNQDGGRSGARPKQRRPQGLPNNTASWFTALTQHGKEDLRFPRGQGVPINTNSSPDDQIGYYRRATRRIRGGDGKMKDLSPRWYFYYLGTGPEAALPYGANKEGIVWVATEGALNTPKDHIGTRNPNNNAAIVLQLPQGTTLPKGFYAEGSRGGSQASSRSSSRSRNSSRNSTPGSSRGTSPARNLQAGGDTALALLLLDRLNQLESKMSGKGQQQQGQTVTKKSAAEASKKPRQKRTATKQYNVTQAFGRRGPEQTQGNFGDQELIRQGTDYKQWPQIAQFAPSASAFFGMSREVTPSGTWLTYTGAIKLDDKDPQFKDNVILLNKHIDAYKTFPPTEPKKDKKKKADEAQPLPQRQKKQQTVTLLPAADLDDFSRQLQNSMSGASADSTQA

SEQ ID NO: 48 - protein, 166 aa, synthetic construct
  REGION 1..166: note = Coronavirus membrane protein designed sequence
MADSNGTITVEELKKLLEQITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNGKLNTDHSSSSDNIALLVQ

SEQ ID NO: 49 - protein, 165 aa, synthetic construct
  REGION 1..165: note = Coronavirus membrane protein designed sequence
MADNGTITVEELKQLLEQVTGGIAIAMACIVGLMWLSYFVASFRLFARTRSMWSFNPETNILLNVPLRGTILTRPLMESELVIGAVIIRGHLRMAGHSLGRCDIKDLPKEITVATSRTLSYYKLGASQRVGTDSGFAAYNRYRIGNGKLNTDHAGSNDNIALLVQ

SEQ ID NO: 50 - protein, 166 aa, synthetic construct
  REGION 1..166: note = Coronavirus membrane protein designed sequence
MADSNGTITVEELKKLLEQVTGGIAIAMACIVGLMWLSYFVASFRLFARTRSMWSFNPETNILLNVPLRGSIITRPLMESELVIGAVILRGHLRMAGHSLGRCDIKDLPKEITVATSRTLSYYKLGASQRVASDSGFAVYNRYRIGNGKLNTDHSSSSDNIALLVQ

SEQ ID NO: 51 - protein, 262 aa, synthetic construct
  REGION 1..262: note = Coronavirus S-protein RBD designed protein
MKRGLCCVLLLCGAVFVSPSAARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNGGSGLNDIFEAQKIEWHEGSHHHHHH

SEQ ID NO: 52 - protein, 1273 aa, synthetic construct; identical to SEQ ID NO: 7
  REGION 1..1273: note = Coronavirus S-protein mutant sequence

SEQ ID NO: 53 - protein, 1270 aa, synthetic construct
  REGION 1..1270: note = Coronavirus "Super_spike" S-protein designed sequence
MFVFLVLLPLVSSQCVNFTNRTQLPSAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 54 - protein, 1270 aa, synthetic construct
  REGION 1..1270: note = Coronavirus S-protein designed sequence
MFVFLVLLPLVSSQCVNFTNRTQLPSAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPCQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPCEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 55 - protein, 284 aa, synthetic construct
  REGION 1..284: note = Designed S protein RBD sequence COV_S_T2_19
RVAPTKEVVRFPNITNLCPFGEVFNATRFPSVYAWERKRISNCVADYSVLYNSTSFSTFKCYGVSPTKLNDLCFTNVYADSFVIRGDEVRQIAPGQTGVIADYNYKLPDDFTGCVIAWNTNNLDSTTGGNYNYLYRSLRKSKLKPFERDISSDIYSPGGKPCSGVEGFNCYYPLRSYGFFPTNGVGYQPYRVVVLSFELLNAPATVCGPKLSTDGGGGSGGGGSGGGGSGGGGSKSSIASFFFIIGLIIGLFLVLRVGIHLCIKLKHTKKRQIYTDIEMNRLGK

SEQ ID NO: 56 - protein, 284 aa, synthetic construct
  REGION 1..284: note = Designed S protein RBD sequence COV_S_T2_20
RVAPTKEVVRFPNITNLCPFGEVFNATKFPSVYAWERKKISNCVADYSVLYNSTSFSTFKCYGVSPTKLNDLCFTNVYADSFVIRGDEVRQIAPGQTGVIADYNYKLPDDFTGCVIAWNTNNIDSTTGGNYNYLYRSLRKSKLKPFERDISSDIYSPGGKPCSGVEGFNCYYPLRSYGFFPTNGTGYQPYRVVVLSFELLNAPATVCGPKLSTDGGGGSGGGGSGGGGSGGGGSKSSIASFFFIIGLIIGLFLVLRVGIHLCIKLKHTKKRQIYTDIEMNRLGK

SEQ ID NO: 57 - protein, 16 aa, synthetic construct
  REGION 1..16: note = Part of discontinuous epitope of COV_S_T2_14 and Part ofdiscontinuous epitope of COV_S_T2_17
NITNLCPFGEVFNATK

SEQ ID NO: 58 - protein, 5 aa, synthetic construct
  REGION 1..5: note = Part of discontinuous epitope of COV_S_T2_14 and COV_S_T2_17
KKISN

SEQ ID NO: 59 - skipped

SEQ ID NO: 60 - protein, 25 aa, synthetic construct
  REGION 1..25: note = Part of discontinuous epitope of COV_S_T2_15 and COV_S_T2_18
YNSTFFSTFKCYGVSPTKLNDLCFS

SEQ ID NO: 61 - protein, 4 aa, synthetic construct
  REGION 1..4: note = Part of discontinuous epitope of COV_S_T2_15 and COV_S_T2_18
DDFM

SEQ ID NO: 62 - protein, 5 aa, synthetic construct
  REGION 1..5: note = Part of discontinuous epitope of COV_S_T2_15 and COV_S_T2_18
FELLN

SEQ ID NO: 63 - protein, 7 aa, synthetic construct
  REGION 1..7: note = Part of discontinuous epitope of COV_S_T2_16
RGDEVRQ

SEQ ID NO: 64 - protein, 7 aa, synthetic construct
  REGION 1..7: note = Part of discontinuous epitope of COV_S_T2_16
TGKIADY

SEQ ID NO: 65 - protein, 8 aa, synthetic construct
  REGION 1..8: note = Part of discontinuous epitope of COV_S_T2_16
YRLFRKSN

SEQ ID NO: 66 - protein, 6 aa, synthetic construct
  REGION 1..6: note = Part of discontinuous epitope of COV_S_T2_16
YQAGST

SEQ ID NO: 67 - protein, 20 aa, synthetic construct
  REGION 1..20: note = Part of discontinuous epitope of COV_S_T2_16
FNCYFPLQSYGFQPTNGVGY

SEQ ID NO: 68 - protein, 16 aa, synthetic construct
  REGION 1..16: note = Part of discontinuous epitope present in COV_S_T2_13
NITNLCPFGEVFNATR

SEQ ID NO: 69 - protein, 5 aa, synthetic construct
  REGION 1..5: note = Part of discontinuous epitope present in COV_S_T2_13
KRISN

SEQ ID NO: 70 - skipped

SEQ ID NO: 71 - protein, 25 aa, synthetic construct
  REGION 1..25: note = Part of discontinuous epitope present in COV_S_T2_13
YNSTSFSTFKCYGVSPTKLNDLCFT

SEQ ID NO: 72 - protein, 4 aa, synthetic construct
  REGION 1..4: note = Part of discontinuous epitope present in COV_S_T2_13
DDFT

SEQ ID NO: 73 - protein, 7 aa, synthetic construct
  REGION 1..7: note = Part of discontinuous epitope present in COV_S_T2_13
TGVIADY

SEQ ID NO: 74 - protein, 8 aa, synthetic construct
  REGION 1..8: note = Part of discontinuous epitope present in COV_S_T2_13
YRSLRKSK

SEQ ID NO: 75 - protein, 6 aa, synthetic construct
  REGION 1..6: note = Part of discontinuous epitope present in COV_S_T2_13
YSPGGK

SEQ ID NO: 76 - protein, 20 aa, synthetic construct
  REGION 1..20: note = Part of discontinuous epitope present in COV_S_T2_13
FNCYYPLRSYGFFPTNGVGY

SEQ ID NO: 77 - protein, 20 aa, synthetic construct
  REGION 1..20: note = Part of discontinuous epitope present in COV_S_T2_13
FNCYYPLRSYGFFPTNGTGY

SEQ ID NO: 78 - DNA, 642 bp, synthetic construct
  misc_feature 1..642: note = COV_S_T2_13 encoding nucleic acid
  source 1..642: mol_type = other DNA
agagtggcccctaccaaagaagtcgtgcggttccccaacatcaccaatctgtgccctttcggcgaggtgttcaacgccaccagatttccctctgtgtacgcctgggagagaaagcggatcagcaactgcgtggccgactacagcgtgctgtacaacagcaccagcttcagcaccttcaagtgctacggcgtgtcacccaccaagctgaacgacctgtgcttcaccaacgtgtacgccgacagcttcgtgatcagaggcgacgaagtgcggcagattgcccctggacaaacaggcgtgatcgccgattacaactacaagctgcccgacgacttcaccggctgtgtgatcgcctggaacaccaacaacctggacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcgtgggctaccagccttacagagtggtggtcctgagcttcgagctgctgaatgcccctgccacagtgtgtggccctaagctgtctaccgac

SEQ ID NO: 79 - DNA, 642 bp, synthetic construct
  misc_feature 1..642: note = COV_S_T2_14 encoding nucleic acid
  source 1..642: mol_type = other DNA
agagtggcccctaccaaagaagtcgtgcggttccccaacatcaccaatctgtgccctttcggcgaggtgttcaacgccaccaagtttccctctgtgtacgcctgggagcgcaaaaagatcagcaactgcgtggccgactacagcgtgctgtacaacagcaccagcttcagcaccttcaagtgctacggcgtgtcacccaccaagctgaacgacctgtgcttcaccaacgtgtacgccgacagcttcgtgatcagaggcgacgaagtgcggcagattgcccctggacaaacaggcgtgatcgccgattacaactacaagctgcccgacgacttcaccggctgtgtgatcgcctggaacaccaacaacatcgacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcgtgggctaccagccttacagagtggtggtcctgagcttcgagctgctgaatgcccctgccacagtgtgtggccctaagctgtctaccgac

SEQ ID NO: 80 - DNA, 642 bp, synthetic construct
  misc_feature 1..642: note = COV_S_T2_15 encoding nucleic acid
  source 1..642: mol_type = other DNA
agagtggcccctaccaaagaagtcgtgcggttccccaacatcaccaatctgtgccctttcggcgaggtgttcaacgccaccagatttccctctgtgtacgcctgggagagaaagcggatcagcaactgcgtggccgactacagcgtgctgtacaacagcaccttcttcagcacctttaagtgctacggcgtgtcacccaccaagctgaacgacctgtgcttcagcaacgtgtacgccgacagcttcgtgatcagaggcgacgaagtgcggcagattgcccctggacaaacaggcgtgatcgccgattacaactacaagctgcccgacgacttcatgggctgtgtgatcgcctggaacaccaacaacctggacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcgtgggctaccagccttacagagtggtggtcctgagcttcgagctgctgaatgcccctgccacagtgtgtggccctaagctgtctaccgac

SEQ ID NO: 81 - DNA, 642 bp, synthetic construct
  misc_feature 1..642: note = COV_S_T2_16 encoding nucleic acid
  source 1..642: mol_type = other DNA
agagtggcccctaccaaagaagtcgtgcggttccccaacatcaccaatctgtgccctttcggcgaggtgttcaacgccaccagatttccctctgtgtacgcctgggagagaaagcggatcagcaactgcgtggccgactacagcgtgctgtacaacagcaccagcttcagcaccttcaagtgctacggcgtgtcacccaccaagctgaacgacctgtgcttcaccaacgtgtacgccgacagcttcgtgatcagaggcgacgaagtgcggcagattgcccctggacagacaggcaagatcgccgattacaactacaagctgcccgacgacttcaccggctgtgtgatcgcctggaacaccaacaacctggacagcaccaccggcggcaactacaactacctgtaccggctgttccggaagtccaacctgaagcctttcgagcgggacatcagcagcgacatctatcaggccggcagcacaccttgttctggcgtggaaggcttcaactgctacttcccactgcaaagctacggcttccagcctaccaacggcgtgggctaccagccttatagagtggtggtcctgagcttcgagctgctgaatgcccctgccacagtgtgtggccctaagctgtctaccgac

SEQ ID NO: 82 - DNA, 642 bp, synthetic construct
  misc_feature 1..642: note = COV_S_T2_17 encoding nucleic acid
  source 1..642: mol_type = other DNA
agagtggcccctaccaaagaagtcgtgcggttccccaacatcaccaatctgtgccctttcggcgaggtgttcaacgccaccaagtttccctctgtgtacgcctgggagcgcaaaaagatcagcaactgcgtggccgactacagcgtgctgtacaacagcaccagcttcagcaccttcaagtgctacggcgtgtcacccaccaagctgaacgacctgtgcttcaccaacgtgtacgccgacagcttcgtgatcagaggcgacgaagtgcggcagattgcccctggacaaacaggcgtgatcgccgattacaactacaagctgcccgacgacttcaccggctgtgtgatcgcctggaacaccaacaacatcgacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcacaggctaccagccttacagagtggtggtcctgagcttcgagctgctgaatgcccctgccacagtgtgtggccctaagctgtctaccgac

SEQ ID NO: 83 - DNA, 642 bp, synthetic construct
  misc_feature 1..642: note = COV_S_T2_18 encoding nucleic acid
  source 1..642: mol_type = other DNA
agagtggcccctaccaaagaagtcgtgcggttccccaacatcaccaatctgtgccctttcggcgaggtgttcaacgccaccagatttccctctgtgtacgcctgggagagaaagcggatcagcaactgcgtggccgactacagcgtgctgtacaacagcaccttcttcagcacctttaagtgctacggcgtgtcacccaccaagctgaacgacctgtgcttcagcaacgtgtacgccgacagcttcgtgatcagaggcgacgaagtgcggcagattgcccctggacaaacaggcgtgatcgccgattacaactacaagctgcccgacgacttcatgggctgtgtgatcgcctggaacaccaacaacctggacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcacaggctaccagccttacagagtggtggtcctgagcttcgagctgctgaatgcccctgccacagtgtgtggccctaagctgtctaccgac

SEQ ID NO: 84 - DNA, 852 bp, synthetic construct
  misc_feature 1..852: note = COV_S_T2_19 encoding nucleic acid
  source 1..852: mol_type = other DNA
agagtggcccctaccaaagaagtcgtgcggttccccaacatcaccaatctgtgccctttcggcgaggtgttcaacgccaccagatttccctctgtgtacgcctgggagagaaagcggatcagcaactgcgtggccgactacagcgtgctgtacaacagcaccagcttcagcaccttcaagtgctacggcgtgtcacccaccaagctgaacgacctgtgcttcaccaacgtgtacgccgacagcttcgtgatcagaggcgacgaagtgcggcagattgcccctggacaaacaggcgtgatcgccgattacaactacaagctgcccgacgacttcaccggctgtgtgatcgcctggaacaccaacaacctggacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcgtgggctaccagccttacagagtggtggtcctgagcttcgagctgctgaatgcccctgccacagtgtgtggccctaagctgtctacagatggcggcggaggatctggcggaggtggaagcggaggcggaggaagcggtggcggcggatctaaatcttctatcgccagcttcttcttcatcatcggcctgattatcggcctgttcctggtgctgagagtgggcatccacctgtgcatcaagctgaaacacaccaagaagcggcaaatctacaccgacatcgagatgaaccggctgggcaaa

SEQ ID NO: 85 - DNA, 852 bp, synthetic construct
  misc_feature 1..852: note = COV_S_T2_20 encoding nucleic acid
  source 1..852: mol_type = other DNA
agagtggcccctaccaaagaagtcgtgcggttccccaacatcaccaatctgtgccctttcggcgaggtgttcaacgccaccaagtttccctctgtgtacgcctgggagcgcaaaaagatcagcaactgcgtggccgactacagcgtgctgtacaacagcaccagcttcagcaccttcaagtgctacggcgtgtcacccaccaagctgaacgacctgtgcttcaccaacgtgtacgccgacagcttcgtgatcagaggcgacgaagtgcggcagattgcccctggacaaacaggcgtgatcgccgattacaactacaagctgcccgacgacttcaccggctgtgtgatcgcctggaacaccaacaacatcgacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcacaggctaccagccttacagagtggtggtcctgagcttcgagctgctgaatgcccctgccacagtgtgtggccctaagctgtctacagatggcggcggaggatctggcggaggtggaagcggaggcggaggaagcggtggcggcggatctaaatcttctatcgccagcttcttcttcatcatcggcctgattatcggcctgttcctggtgctgagagtgggcatccacctgtgcatcaagctgaaacacaccaagaagcggcaaatctacaccgacatcgagatgaaccggctgggcaaa

SEQ ID NO: 86 - DNA, 5102 bp, synthetic construct
  source 1..5102: mol_type = other DNA
cctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcggtaccgccaccatggatgctatgaagaggggcctgtgctgcgtgctgcttctgtgtggcgctgtgtttgtgtctcctagcgccgctagagtggcccctaccaaagaagtcgtgcggttccccaacatcaccaatctgtgccctttcggcgaggtgttcaacgccaccaagtttccctctgtgtacgcctgggagcgcaaaaagatcagcaactgcgtggccgactacagcgtgctgtacaacagcaccagcttcagcaccttcaagtgctacggcgtgtcacccaccaagctgaacgacctgtgcttcaccaacgtgtacgccgacagcttcgtgatcagaggcgacgaagtgcggcagattgcccctggacaaacaggcgtgatcgccgattacaactacaagctgcccgacgacttcaccggctgtgtgatcgcctggaacaccaacaacatcgacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcacaggctaccagccttacagagtggtggtcctgagcttcgagctgctgaatgcccctgccacagtgtgtggccctaagctgtctaccgactgagcggccgcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaa

SEQ ID NO: 87 - protein, 1270 aa, synthetic construct
MFVFLVLLPLVSSQCVNFTNRTQLPSAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFRPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 88 - protein, 1251 aa, synthetic construct
MFVFLVLLPLVSSQCVNFTNRTQLPSAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFRPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 89 - DNA, 3810 bp, synthetic construct
  source 1..3810: mol_type = other DNA
atgttcgtgtttctggtgctgctgcctctggtgtccagccagtgcgtgaacttcaccaacagaacccagctgcctagcgcctacaccaacagctttaccagaggcgtgtactaccccgacaaggtgttcagatccagcgtgctgcactctacccaggacctgttcctgcctttcttcagcaacgtgacctggttccacgccatcagcggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtgtaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaaaatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactacgtgggatacctccagcctagaaccttcctgctgaagtacaacgagaacggcaccatcaccgacgccgtggattgtgctctggaccctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagaccggcaatatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatcagcaccgaaatctatcaggccggcagcaccccttgcaatggcgtgaagggctttaactgctacttcccactgcaaagctacggcttccagccaacatacggcgtgggctaccagccttacagagtggtggtgctgagcttcgagctgctgcatgctcctgccacagtgtgcggccctaagaaatccaccaatctcgtgaagaacaaatgcgtcaacttcaatttcaacggcctgaccggcaccggcgtgctgacagagagcaacaagaagttcctgccattccagcagttcggccgggacattgccgataccacagatgccgtcagagatccccagacactggaaatcctggacatcaccccatgcagcttcggcggagtgtctgtgatcacccctggcaccaacaccagcaatcaggtggcagtgctgtaccagggcgtcaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgcgcctcttaccagacacagaccaacagccacagacgggctagaagcgtggccagccagagcatcattgcctacacaatgtctctgggcgccgagaacagcgtggcctactccaacaactctatcgctatccccaccaatttcaccatcagcgtgaccaccgagatcctgcctgtgtccatgaccaagaccagcgtggactgcaccatgtacatctgcggcgattccaccgagtgctccaacctgctgctccagtacggcagcttctgcacccagctgaatagagccctgacagggatcgccgtggaacaggacaagaacacccaagaggtgttcgcccaagtgaagcaaatctacaagacccctcctatcaaggacttcggcggcttcaacttcagccagattctgcccgatcctagcaagcccagcaagcggagcttcatcgaggacctgctgttcaacaaagtgacactggccgacgccggcttcatcaagcagtacggcgattgtctgggcgacattgcagccagggatctgatctgcgcccagaagtttaacggactgacagtgctgcctcctctgctgaccgatgagatgatcgcccagtacacatctgccctgctggccggcacaatcacaagcggctggacatttggagctggcgccgctctccagattccattcgctatgcagatggcctacaggttcaacggcatcggagtgacccagaatgtgctgtacgagaaccagaagctgatcgccaaccagttcaacagcgccatcggcaagatccaggacagcctgagcagcacagcaagcgccctgggaaagctccaggacgtggtcaaccagaatgcccaggcactgaacaccctggtcaagcagctgtcctccaacttcggcgccatctctagcgtgctgaacgatatcctgagcagactggacccacctgaggccgaggtgcagatcgacagactgatcacaggccggctccagagcctccagacatacgttacacagcagctgatcagagccgccgagattagagcctctgccaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaactttaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtccggcaactgcgacgtcgtgatcggcattgtgaacaataccgtgtacgaccctctccagccggaactggactccttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccttggtacatctggctgggctttatcgccggactgattgccatcgtgatggtcacaatcatgctgtgttgcatgaccagctgttgcagctgcctgaagggctgctgtagctgtggctcctgctgcaagttcgacgaggacgattctgagcccgtgctgaagggcgtgaaactgcactacacc

SEQ ID NO: 90 - protein, 3810 aa, synthetic construct
ATGTTCGTGTTTCTGGTGCTGCTGCCTCTGGTGTCCAGCCAGTGCGTGAACTTCACCAACAGAACCCAGCTGCCTAGCGCCTACACCAACAGCTTTACCAGAGGCGTGTACTACCCCGACAAGGTGTTCAGATCCAGCGTGCTGCACTCTACCCAGGACCTGTTCCTGCCTTTCTTCAGCAACGTGACCTGGTTCCACGCCATCAGCGGCACCAATGGCACCAAGAGATTCGACAACCCCGTGCTGCCCTTCAACGACGGGGTGTACTTTGCCAGCACCGAGAAGTCCAACATCATCAGAGGCTGGATCTTCGGCACCACACTGGACAGCAAGACCCAGAGCCTGCTGATCGTGAACAACGCCACCAACGTGGTCATCAAAGTGTGCGAGTTCCAGTTCTGCAACGACCCCTTCCTGGGCGTGTACCACAAGAACAACAAGAGCTGGATGGAAAGCGAGTTCCGGGTGTACAGCAGCGCCAACAACTGCACCTTCGAGTACGTGTCCCAGCCTTTCCTGATGGACCTGGAAGGCAAGCAGGGCAACTTCAAGAACCTGCGCGAGTTCGTGTTCAAGAACATCGACGGCTACTTCAAAATCTACAGCAAGCACACCCCTATCAACCTCGTGCGGGATCTGCCTCAGGGCTTCTCTGCTCTGGAACCCCTGGTGGATCTGCCCATCGGCATCAACATCACCCGGTTTCAGACACTGCTGGCCCTGCACAGAAGCTACCTGACACCTGGCGATAGCAGCAGCGGATGGACAGCTGGTGCCGCCGCTTACTACGTGGGATACCTCCAGCCTAGAACCTTCCTGCTGAAGTACAACGAGAACGGCACCATCACCGACGCCGTGGATTGTGCTCTGGACCCTCTGAGCGAGACAAAGTGCACCCTGAAGTCCTTCACCGTGGAAAAGGGCATCTACCAGACCAGCAACTTCCGGGTGCAGCCCACCGAATCCATCGTGCGGTTCCCCAATATCACCAATCTGTGCCCCTTCGGCGAGGTGTTCAATGCCACCAGATTCGCCTCTGTGTACGCCTGGAACCGGAAGCGGATCAGCAATTGCGTGGCCGACTACTCCGTGCTGTACAACTCCGCCAGCTTCAGCACCTTCAAGTGCTACGGCGTGTCCCCTACCAAGCTGAACGACCTGTGCTTCACAAACGTGTACGCCGACAGCTTCGTGATCCGGGGAGATGAAGTGCGGCAGATTGCCCCTGGACAGACCGGCAATATCGCCGACTACAACTACAAGCTGCCCGACGACTTCACCGGCTGTGTGATTGCCTGGAACAGCAACAACCTGGACTCCAAAGTCGGCGGCAACTACAATTACCTGTACCGGCTGTTCCGGAAGTCCAATCTGAAGCCCTTCGAGCGGGACATCAGCACCGAAATCTATCAGGCCGGCAGCACCCCTTGCAATGGCGTGAAGGGCTTTAACTGCTACTTCCCACTGCAAAGCTACGGCTTCCGGCCAACATACGGCGTGGGCTACCAGCCTTACAGAGTGGTGGTGCTGAGCTTCGAGCTGCTGCATGCTCCTGCCACAGTGTGCGGCCCTAAGAAATCCACCAATCTCGTGAAGAACAAATGCGTCAACTTCAATTTCAACGGCCTGACCGGCACCGGCGTGCTGACAGAGAGCAACAAGAAGTTCCTGCCATTCCAGCAGTTCGGCCGGGACATTGCCGATACCACAGATGCCGTCAGAGATCCCCAGACACTGGAAATCCTGGACATCACCCCATGCAGCTTCGGCGGAGTGTCTGTGATCACCCCTGGCACCAACACCAGCAATCAGGTGGCAGTGCTGTACCAGGGCGTCAACTGTACAGAGGTGCCAGTGGCCATTCACGCCGATCAGCTGACCCCTACTTGGCGGGTGTACTCCACAGGCAGCAATGTGTTTCAGACCAGAGCCGGCTGTCTGATCGGAGCCGAGCACGTGAACAATAGCTACGAGTGCGACATCCCCATCGGCGCTGGCATCTGCGCCTCTTACCAGACACAGACCAACAGCCACAGACGGGCTAGAAGCGTGGCCAGCCAGAGCATCATTGCCTACACAATGTCTCTGGGCGCCGAGAACAGCGTGGCCTACTCCAACAACTCTATCGCTATCCCCACCAATTTCACCATCAGCGTGACCACCGAGATCCTGCCTGTGTCCATGACCAAGACCAGCGTGGACTGCACCATGTACATCTGCGGCGATTCCACCGAGTGCTCCAACCTGCTGCTCCAGTACGGCAGCTTCTGCACCCAGCTGAATAGAGCCCTGACAGGGATCGCCGTGGAACAGGACAAGAACACCCAAGAGGTGTTCGCCCAAGTGAAGCAAATCTACAAGACCCCTCCTATCAAGGACTTCGGCGGCTTCAACTTCAGCCAGATTCTGCCCGATCCTAGCAAGCCCAGCAAGCGGAGCTTCATCGAGGACCTGCTGTTCAACAAAGTGACACTGGCCGACGCCGGCTTCATCAAGCAGTACGGCGATTGTCTGGGCGACATTGCAGCCAGGGATCTGATCTGCGCCCAGAAGTTTAACGGACTGACAGTGCTGCCTCCTCTGCTGACCGATGAGATGATCGCCCAGTACACATCTGCCCTGCTGGCCGGCACAATCACAAGCGGCTGGACATTTGGAGCTGGCGCCGCTCTCCAGATTCCATTCGCTATGCAGATGGCCTACAGGTTCAACGGCATCGGAGTGACCCAGAATGTGCTGTACGAGAACCAGAAGCTGATCGCCAACCAGTTCAACAGCGCCATCGGCAAGATCCAGGACAGCCTGAGCAGCACAGCAAGCGCCCTGGGAAAGCTCCAGGACGTGGTCAACCAGAATGCCCAGGCACTGAACACCCTGGTCAAGCAGCTGTCCTCCAACTTCGGCGCCATCTCTAGCGTGCTGAACGATATCCTGAGCAGACTGGACCCACCTGAGGCCGAGGTGCAGATCGACAGACTGATCACAGGCCGGCTCCAGAGCCTCCAGACATACGTTACACAGCAGCTGATCAGAGCCGCCGAGATTAGAGCCTCTGCCAATCTGGCCGCCACCAAGATGTCTGAGTGTGTGCTGGGCCAGAGCAAGAGAGTGGACTTTTGCGGCAAGGGCTACCACCTGATGAGCTTCCCTCAGTCTGCACCACACGGCGTGGTGTTTCTGCACGTGACATACGTGCCCGCTCAAGAGAAGAACTTTACCACCGCTCCAGCCATCTGCCACGACGGCAAAGCCCACTTTCCTAGAGAAGGCGTGTTCGTGTCCAACGGCACCCATTGGTTCGTGACCCAGCGGAACTTCTACGAGCCCCAGATCATCACCACCGACAACACCTTCGTGTCCGGCAACTGCGACGTCGTGATCGGCATTGTGAACAATACCGTGTACGACCCTCTCCAGCCGGAACTGGACTCCTTCAAAGAGGAACTGGATAAGTACTTTAAGAACCACACAAGCCCCGACGTGGACCTGGGCGATATCAGCGGAATCAATGCCAGCGTCGTGAACATCCAGAAAGAGATCGACCGGCTGAACGAGGTGGCCAAGAATCTGAACGAGAGCCTGATCGACCTGCAAGAACTGGGGAAGTACGAGCAGTACATCAAGTGGCCTTGGTACATCTGGCTGGGCTTTATCGCCGGACTGATTGCCATCGTGATGGTCACAATCATGCTGTGTTGCATGACCAGCTGTTGCAGCTGCCTGAAGGGCTGCTGTAGCTGTGGCTCCTGCTGCAAGTTCGACGAGGACGATTCTGAGCCCGTGCTGAAGGGCGTGAAACTGCACTACACC

SEQ ID NO: 91 - DNA, 3753 bp, synthetic construct
  source 1..3753: mol_type = other DNA
atgttcgtgtttctggtgctgctgcctctggtgtccagccagtgcgtgaacttcaccaacagaacccagctgcctagcgcctacaccaacagctttaccagaggcgtgtactaccccgacaaggtgttcagatccagcgtgctgcactctacccaggacctgttcctgcctttcttcagcaacgtgacctggttccacgccatcagcggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtgtaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaaaatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactacgtgggatacctccagcctagaaccttcctgctgaagtacaacgagaacggcaccatcaccgacgccgtggattgtgctctggaccctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagaccggcaatatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatcagcaccgaaatctatcaggccggcagcaccccttgcaatggcgtgaagggctttaactgctacttcccactgcaaagctacggcttccggccaacatacggcgtgggctaccagccttacagagtggtggtgctgagcttcgagctgctgcatgctcctgccacagtgtgcggccctaagaaatccaccaatctcgtgaagaacaaatgcgtcaacttcaatttcaacggcctgaccggcaccggcgtgctgacagagagcaacaagaagttcctgccattccagcagttcggccgggacattgccgataccacagatgccgtcagagatccccagacactggaaatcctggacatcaccccatgcagcttcggcggagtgtctgtgatcacccctggcaccaacaccagcaatcaggtggcagtgctgtaccagggcgtcaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgcgcctcttaccagacacagaccaacagccacagacgggctagaagcgtggccagccagagcatcattgcctacacaatgtctctgggcgccgagaacagcgtggcctactccaacaactctatcgctatccccaccaatttcaccatcagcgtgaccaccgagatcctgcctgtgtccatgaccaagaccagcgtggactgcaccatgtacatctgcggcgattccaccgagtgctccaacctgctgctccagtacggcagcttctgcacccagctgaatagagccctgacagggatcgccgtggaacaggacaagaacacccaagaggtgttcgcccaagtgaagcaaatctacaagacccctcctatcaaggacttcggcggcttcaacttcagccagattctgcccgatcctagcaagcccagcaagcggagcttcatcgaggacctgctgttcaacaaagtgacactggccgacgccggcttcatcaagcagtacggcgattgtctgggcgacattgcagccagggatctgatctgcgcccagaagtttaacggactgacagtgctgcctcctctgctgaccgatgagatgatcgcccagtacacatctgccctgctggccggcacaatcacaagcggctggacatttggagctggcgccgctctccagattccattcgctatgcagatggcctacaggttcaacggcatcggagtgacccagaatgtgctgtacgagaaccagaagctgatcgccaaccagttcaacagcgccatcggcaagatccaggacagcctgagcagcacagcaagcgccctgggaaagctccaggacgtggtcaaccagaatgcccaggcactgaacaccctggtcaagcagctgtcctccaacttcggcgccatctctagcgtgctgaacgatatcctgagcagactggacccacctgaggccgaggtgcagatcgacagactgatcacaggccggctccagagcctccagacatacgttacacagcagctgatcagagccgccgagattagagcctctgccaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaactttaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtccggcaactgcgacgtcgtgatcggcattgtgaacaataccgtgtacgaccctctccagccggaactggactccttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccttggtacatctggctgggctttatcgccggactgattgccatcgtgatggtcacaatcatgctgtgttgcatgaccagctgttgcagctgcctgaagggctgctgtagctgtggctcctgctgc

SEQ ID NO: 92 - protein, 239 aa, synthetic construct
MDAMKRGLCCVLLLCGAVFVSPSAARVAPTKEVVRFPNITNLCPFGEVFNATKFPSVYAWERKKISNCVADYSVLYNSTSFSTFKCYGVSPTKLNDLCFTNVYADSFVIRGDEVRQIAPGQTGVIADYNYKLPDDFTGCVIAWNTNNIDSTTGGNYNYLYRSLRKSKLKPFERDISSDIYSPGGKPCSGVEGFNCYYPLRSYGFFPTNGTGYQPYRVVVLSFELLNAPATVCGPKLSTD

SEQ ID NO: 93 - DNA, 720 bp, synthetic construct
  source 1..720: mol_type = other DNA
atggatgctatgaagaggggcctgtgctgcgtgctgcttctgtgtggcgctgtgtttgtgtctcctagcgccgctagagtggcccctaccaaagaagtcgtgcggttccccaacatcaccaatctgtgccctttcggcgaggtgttcaacgccaccaagtttccctctgtgtacgcctgggagcgcaaaaagatcagcaactgcgtggccgactacagcgtgctgtacaacagcaccagcttcagcaccttcaagtgctacggcgtgtcacccaccaagctgaacgacctgtgcttcaccaacgtgtacgccgacagcttcgtgatcagaggcgacgaagtgcggcagattgcccctggacaaacaggcgtgatcgccgattacaactacaagctgcccgacgacttcaccggctgtgtgatcgcctggaacaccaacaacatcgacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcacaggctaccagccttacagagtggtggtcctgagcttcgagctgctgaatgcccctgccacagtgtgtggccctaagctgtctaccgactga

SEQ ID NO: 94 - DNA, 5102 bp, synthetic construct
  source 1..5102: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcggtaccgccaccatggatgctatgaagaggggcctgtgctgcgtgctgcttctgtgtggcgctgtgtttgtgtctcctagcgccgctagagtggcccctaccaaagaagtcgtgcggttccccaacatcaccaatctgtgccctttcggcgaggtgttcaacgccaccaagtttccctctgtgtacgcctgggagcgcaaaaagatcagcaactgcgtggccgactacagcgtgctgtacaacagcaccagcttcagcaccttcaagtgctacggcgtgtcacccaccaagctgaacgacctgtgcttcaccaacgtgtacgccgacagcttcgtgatcagaggcgacgaagtgcggcagattgcccctggacaaacaggcgtgatcgccgattacaactacaagctgcccgacgacttcaccggctgtgtgatcgcctggaacaccaacaacatcgacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcacaggctaccagccttacagagtggtggtcctgagcttcgagctgctgaatgcccctgccacagtgtgtggccctaagctgtctaccgactgagcggccgcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 95 - DNA, 8138 bp, synthetic construct
  source 1..8138: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcggtaccgccaccatgttcgtgtttctggtgctgctgcctctggtgtccagccagtgcgtgaacttcaccaacagaacccagctgcctagcgcctacaccaacagctttaccagaggcgtgtactaccccgacaaggtgttcagatccagcgtgctgcactctacccaggacctgttcctgcctttcttcagcaacgtgacctggttccacgccatcagcggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtgtaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaaaatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactacgtgggatacctccagcctagaaccttcctgctgaagtacaacgagaacggcaccatcaccgacgccgtggattgtgctctggaccctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagaccggcaatatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatcagcaccgaaatctatcaggccggcagcaccccttgcaatggcgtgaagggctttaactgctacttcccactgcaaagctacggcttccggccaacatacggcgtgggctaccagccttacagagtggtggtgctgagcttcgagctgctgcatgctcctgccacagtgtgcggccctaagaaatccaccaatctcgtgaagaacaaatgcgtcaacttcaatttcaacggcctgaccggcaccggcgtgctgacagagagcaacaagaagttcctgccattccagcagttcggccgggacattgccgataccacagatgccgtcagagatccccagacactggaaatcctggacatcaccccatgcagcttcggcggagtgtctgtgatcacccctggcaccaacaccagcaatcaggtggcagtgctgtaccagggcgtcaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgcgcctcttaccagacacagaccaacagccacagacgggctagaagcgtggccagccagagcatcattgcctacacaatgtctctgggcgccgagaacagcgtggcctactccaacaactctatcgctatccccaccaatttcaccatcagcgtgaccaccgagatcctgcctgtgtccatgaccaagaccagcgtggactgcaccatgtacatctgcggcgattccaccgagtgctccaacctgctgctccagtacggcagcttctgcacccagctgaatagagccctgacagggatcgccgtggaacaggacaagaacacccaagaggtgttcgcccaagtgaagcaaatctacaagacccctcctatcaaggacttcggcggcttcaacttcagccagattctgcccgatcctagcaagcccagcaagcggagcttcatcgaggacctgctgttcaacaaagtgacactggccgacgccggcttcatcaagcagtacggcgattgtctgggcgacattgcagccagggatctgatctgcgcccagaagtttaacggactgacagtgctgcctcctctgctgaccgatgagatgatcgcccagtacacatctgccctgctggccggcacaatcacaagcggctggacatttggagctggcgccgctctccagattccattcgctatgcagatggcctacaggttcaacggcatcggagtgacccagaatgtgctgtacgagaaccagaagctgatcgccaaccagttcaacagcgccatcggcaagatccaggacagcctgagcagcacagcaagcgccctgggaaagctccaggacgtggtcaaccagaatgcccaggcactgaacaccctggtcaagcagctgtcctccaacttcggcgccatctctagcgtgctgaacgatatcctgagcagactggacccacctgaggccgaggtgcagatcgacagactgatcacaggccggctccagagcctccagacatacgttacacagcagctgatcagagccgccgagattagagcctctgccaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaactttaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtccggcaactgcgacgtcgtgatcggcattgtgaacaataccgtgtacgaccctctccagccggaactggactccttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccttggtacatctggctgggctttatcgccggactgattgccatcgtgatggtcacaatcatgctgtgttgcatgaccagctgttgcagctgcctgaagggctgctgtagctgtggctcctgctgctgagcggccgcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 96 - DNA, 1711 bp, synthetic construct
  source 1..1711: mol_type = other DNA
ctgaatatgaaggagcaaaaggttgtaacattttattaccgtgtgggatataaaagtccttgatccattgatctggaaacgggcatctccatttaagactagacgccacggggtttaaaatactaatcatgacattttgtagagcgtaattacttagtaaatccgccgtactaggttcatttcctcctcgtttggatctcacatcagaaattaaaataatcttagaaggatgcagttgttttttgatggatcgtagatattcctcatcaacgaaccgagtcactagagtcacatcacgcaatccatttaaaataggatcatgatggcggccgtcaattagcatccatttgatgatcactcctaaattatagaaatgatctctcaaataacgtatatgtgtaccgggagcagatcctatatacactacggtggcaccatctaatataccgtgtcgctgtaacttactaagaaaaaataattctcctagtaatagttttaactgtccttgatacggcagtttttttgcgacctcatttgcactttctggttcgtaatctaactcattatcaatttcctcaaaatacataaacggtttatctaacgacacaacatccatttttaagtattatattaaaatttaatcaatgtttatttttagttttttagataaaaaatataatattatgagccgacgtaacactttctacacaccgattgatacatatcattacctcctattatctctatctcggtttcctcacccaatcgtttagaaaaggaagcctccttaaagcatttcatacacacagcagttagttttaccaccatttcagataatggaataagattcaaaatattattaaacggtttacgttgaaatgtcccatcgagtgcggctactataactatttttccttcgtttgccatacgctcacagaattcaacaatgtctggaaagaactgtccttcatcgatacctatcacggagaaatctgtaattgattccaagacatcacatagtttagttgcttccaatgcttcaaaattattcttatcatgcgtccatagtcccgttccgtatctattatcgttagaatattttatagtcacgcatttatattgagctatttgataacgtctaactcgtctaattaattctgtacttttacctgaaaacatggggccgattatcaactgaatatgtccgccgttcatgatgacaataaagaattaattattgttcactttattcgactttaatatatccatcacgttagaaaatgcgatatcgcgacgaggatctatgtatctaataggatctattgcggtggtagctagagaggattcttttttgaatcgcatcaaactaatcacaaagtcgaacaaatatcctttattaagtttgacccttccatctgtaacaatagggaccttgttaaacagttttttaaaatcttgaaagtctgtgaattttgtcaattgtctgtattcctctgaaagagattcataacaatgacccacggcttctaatttattttttgattggatcaataataataacagaaagtctagatattgagtgatttgcaatatatcagataatgaagattcatcatcttgactagccaaatacttaaaaaatgaatcatcatctgcgaagaacatcgttaagagatactggttgtgatccatttattgatcgcaaaagctt

SEQ ID NO: 97 - DNA, 817 bp, synthetic construct
  source 1..817: mol_type = other DNA
aaaaaatgaaaataaatacaaaggttcttgagggttgtgttaaattgaaagcgagaaataatcataaattggtaccgccaccatggatgctatgaagaggggcctgtgctgcgtgctgcttctgtgtggcgctgtgtttgtgtctcctagcgccgctagagtggcccctaccaaagaagtcgtgcggttccccaacatcaccaatctgtgccctttcggcgaggtgttcaacgccaccaagtttccctctgtgtacgcctgggagcgcaaaaagatcagcaactgcgtggccgactacagcgtgctgtacaacagcaccagcttcagcaccttcaagtgctacggcgtgtcacccaccaagctgaacgacctgtgcttcaccaacgtgtacgccgacagcttcgtgatcagaggcgacgaagtgcggcagattgcccctggacaaacaggcgtgatcgccgattacaactacaagctgcccgacgacttcaccggctgtgtgatcgcctggaacaccaacaacatcgacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcacaggctaccagccttacagagtggtggtcctgagcttcgagctgctgaatgcccctgccacagtgtgtggccctaagctgtctaccgactgagcggccgctttttat

SEQ ID NO: 98 - DNA, 3853 bp, synthetic construct
  source 1..3853: mol_type = other DNA
aaaaaatgaaaataaatacaaaggttcttgagggttgtgttaaattgaaagcgagaaataatcataaattggtaccgccaccatgttcgtgtttctggtgctgctgcctctggtgtccagccagtgcgtgaacttcaccaacagaacccagctgcctagcgcctacaccaacagctttaccagaggcgtgtactaccccgacaaggtgttcagatccagcgtgctgcactctacccaggacctgttcctgcctttcttcagcaacgtgacctggttccacgccatcagcggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtgtaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaaaatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactacgtgggatacctccagcctagaaccttcctgctgaagtacaacgagaacggcaccatcaccgacgccgtggattgtgctctggaccctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagaccggcaatatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatcagcaccgaaatctatcaggccggcagcaccccttgcaatggcgtgaagggctttaactgctacttcccactgcaaagctacggcttccggccaacatacggcgtgggctaccagccttacagagtggtggtgctgagcttcgagctgctgcatgctcctgccacagtgtgcggccctaagaaatccaccaatctcgtgaagaacaaatgcgtcaacttcaatttcaacggcctgaccggcaccggcgtgctgacagagagcaacaagaagttcctgccattccagcagttcggccgggacattgccgataccacagatgccgtcagagatccccagacactggaaatcctggacatcaccccatgcagcttcggcggagtgtctgtgatcacccctggcaccaacaccagcaatcaggtggcagtgctgtaccagggcgtcaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgcgcctcttaccagacacagaccaacagccacagacgggctagaagcgtggccagccagagcatcattgcctacacaatgtctctgggcgccgagaacagcgtggcctactccaacaactctatcgctatccccaccaatttcaccatcagcgtgaccaccgagatcctgcctgtgtccatgaccaagaccagcgtggactgcaccatgtacatctgcggcgattccaccgagtgctccaacctgctgctccagtacggcagcttctgcacccagctgaatagagccctgacagggatcgccgtggaacaggacaagaacacccaagaggtgttcgcccaagtgaagcaaatctacaagacccctcctatcaaggacttcggcggcttcaacttcagccagattctgcccgatcctagcaagcccagcaagcggagcttcatcgaggacctgctgttcaacaaagtgacactggccgacgccggcttcatcaagcagtacggcgattgtctgggcgacattgcagccagggatctgatctgcgcccagaagtttaacggactgacagtgctgcctcctctgctgaccgatgagatgatcgcccagtacacatctgccctgctggccggcacaatcacaagcggctggacatttggagctggcgccgctctccagattccattcgctatgcagatggcctacaggttcaacggcatcggagtgacccagaatgtgctgtacgagaaccagaagctgatcgccaaccagttcaacagcgccatcggcaagatccaggacagcctgagcagcacagcaagcgccctgggaaagctccaggacgtggtcaaccagaatgcccaggcactgaacaccctggtcaagcagctgtcctccaacttcggcgccatctctagcgtgctgaacgatatcctgagcagactggacccacctgaggccgaggtgcagatcgacagactgatcacaggccggctccagagcctccagacatacgttacacagcagctgatcagagccgccgagattagagcctctgccaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaactttaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtccggcaactgcgacgtcgtgatcggcattgtgaacaataccgtgtacgaccctctccagccggaactggactccttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccttggtacatctggctgggctttatcgccggactgattgccatcgtgatggtcacaatcatgctgtgttgcatgaccagctgttgcagctgcctgaagggctgctgtagctgtggctcctgctgctgagcggccgctttttat

SEQ ID NO: 99 - protein, 91 aa, synthetic construct
  REGION 1..91: note = SARS1 S protein
VFQTQAGCLIGAEHVDTSYECDIPIGAGICASYHTVSLLRSTSQKSIVAYTMSLGADSSIAYSNNTIAIPTNFSISITTEVMPVSMAKTSV

SEQ ID NO: 100 - protein, 91 aa, synthetic construct
  REGION 1..91: note = RaTG13 S protein
VFQTRAGCLIGAEHVNNSYECDIPIGAGICASYQTQTNSRSVASQSIIAYTMSLGAENSVAYSNNSIAIPTNFTISVTTEILPVSMTKTSV

SEQ ID NO: 101 - protein, 95 aa, synthetic construct
  REGION 1..95: note = EPI_ISL_402119
VFQTRAGCLIGAEHVNNSYECDIPIGAGICASYQTQTNSPRRARSVASQSIIAYTMSLGAENSVAYSNNSIAIPTNFTISVTTEILPVSMTKTSV

SEQ ID NO: 102 - protein, 95 aa, synthetic construct; identical to SEQ ID NO: 101
  REGION 1..95: note = EPI_ISL_402132

SEQ ID NO: 103 - protein, 95 aa, synthetic construct; identical to SEQ ID NO: 101
  REGION 1..95: note = EPI_ISL_403936

SEQ ID NO: 104 - protein, 95 aa, synthetic construct; identical to SEQ ID NO: 101
  REGION 1..95: note = EPI_ISL_404253

SEQ ID NO: 105 - protein, 95 aa, synthetic construct; identical to SEQ ID NO: 101
  REGION 1..95: note = Consensus S protein

SEQ ID NO: 106 - protein, 77 aa, synthetic construct
  REGION 1..77: note = JQ765573_Human_coronavirus_NL63_Alpha E protein
MFLRLIDDNGIVLNSILWLLVMIFFFVLAMTFIKLIQLCFTCHYFFSRTLYQPVYKIFLAYQDYMQIAPVPAEVLNV

SEQ ID NO: 107 - protein, 77 aa, synthetic construct
  REGION 1..77: note = KF514430_Human_coronavirus_229E_Alpha E protein
MFLKLVDDHALVVNVLLWCVVLIVILLVCITIIKLIKLCFTCHMFCNRTVYGPIKNVYHIYQSYMHIDPFPKRVIDF

SEQ ID NO: 108 - protein, 82 aa, synthetic construct
  REGION 1..82: note = KF686340_Human_coronavirus_HKU1_Beta E protein
MVDLFFNDTAWYIGQILVLVLFCLISLIFVVAFLATIKLCMQLCGFCNFFIISPSAYVYKRGMQLYKSYSEQVIPPTSDYLI

SEQ ID NO: 109 - protein, 82 aa, synthetic construct
  REGION 1..82: note = MH940245_Human_coronavirus_HKU1_Beta E protein
MVDVFFTDTAWYVGQIFFLVLSCVIFLIFVVALLATIKLCIQICGFCNIFIISPSAYVYNRGRQLYKSYSEHVIPSTLDDLI

SEQ ID NO: 110 - protein, 82 aa, synthetic construct
  REGION 1..82: note = KF600630_MERS_Beta E protein
MLPFVQERVGLFIVNFFIFTVVCAITLLVCMAFLTATRLCVQCMTGFNTLLVQPALYLYNTGRSVYVKFQDSKPPLPPDEWV

SEQ ID NO: 111 - protein, 59 aa, synthetic construct
  REGION 1..59: note = Consensus E protein
MLVETGLIVNSLLLVCVFLLVALTAILCQCCFCNLVPSAYVYRGLYSYEIVPPPPDLLV

SEQ ID NO: 112 - protein, 57 aa, synthetic construct
  REGION 1..57: note = Consensus E protein
MFVETGIVNLLLACVIFLLVALTARLCAQCCNVLVPSAYVYRGLYSYSEVIPPDLLV

SEQ ID NO: 113 - protein, 73 aa, synthetic construct
  REGION 1..73: note = Consensus E protein
MYSFVSEETGTLIVNSVLLFLAFVVFLLVTLAILTALRLCAYCCNIVNVSLVKPYVYSRVKNLNSSGVPDLLV

SEQ ID NO: 114 - protein, 221 aa, synthetic construct
  REGION 1..221: note = SARS-CoV-2 M protein
MADNGTITVEELKQLLEQWNLVIGFLFLAWIMMLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 115 - protein, 213 aa, synthetic construct
  REGION 1..213: note = WIV16 S protein RBD
RVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLSTD

SEQ ID NO: 116 - protein, 214 aa, synthetic construct
  REGION 1..214: note = RaTG13 S protein RBD
RVQPTDSIVRFPNITNLCPFGEVFNATTFASVYAWNRKRISNCVADYSVLYNSTSFSTFKCYGVSPTKLNDLCFTNVYADSFVITGDEVRQIAPGQTGKIADYNYKLPDDFTGCVIAWNSKHIDAKEGGNFNYLYRLFRKANLKPFERDISTEIYQAGSKPCNGQTGLNCYYPLYRYGFYPTDGVGHQPYRVVVLSFELLNAPATVCGPKKSTN

SEQ ID NO: 117 - protein, 215 aa, synthetic construct
  REGION 1..215: note = SARS-CoV-2 N1 protein
RVQPTESIVRFPNITNLCPFGEVFNATRFASVYAWNRKRISNCCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYYPLQSYGFQPTNGVGYQPYRVVVLSFELLHAPATVCGPKKSTN

SEQ ID NO: 118 - protein, 215 aa, synthetic construct
  REGION 1..215: note = SARS-CoV-1 N protein
RVVPSGDVVRFPNITNLCPFGEVFNATKFPSVYAWERKKISNCCVADYSVLYNSTTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTD

SEQ ID NO: 119 - protein, 215 aa, synthetic construct
  REGION 1..215: note = EPI_ISL_402119_RBD with C-terminal Lysine residue
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNL

(19) United States
(12) Patent Application Publication
(10) Pub. No.: US 2025/0312437 A1
(43) Pub. Date: Oct. 9, 2025

(54) CORONAVIRUS VACCINES

(71) Applicants: DIOSynVax Ltd, Hertford Hertfordshire (GB); The Chancellor, Masters and Scholars of the University of Cambridge, Cambridge Cambridgeshire (GB); Universitaet Regensburg, Regensburg (DE)

(72) Inventors: Jonathan Luke HEENEY, Cambridge Cambridgeshire (GB); Sneha VISHWANATH, Cambridge Cambridgeshire (GB); George CARNELL, Cambridge Cambridgeshire (GB); David WELLS, Hertford Hertfordshire (GB); Matteo FERRARI, Hertford Hertfordshire (GB); Benedikt ASBACH, Regensburg (DE); Ralf WAGNER, Regensburg (DE); Martina BILLMEIER, Regensburg (DE); Patrick NECKERMANN, Regensburg (DE)

(21) Appl. No.: 18/699,034

(22) PCT Filed: Oct. 6, 2022
(86) PCT No.: PCT/GB2022/052537

§ 371 Date: Apr. 5, 2024

(30) Foreign Application Priority Data
Oct. 6, 2021 (GB) 2114318.5
Sep. 27, 2022 (GB) 2214126.1

(51) Int. Cl. A61K 39/215; A61K 39/00; A61P 35/00; C07K 14/005; C12N 15/86

(52) U.S. Cl. CPC A61K 39/215; A61P 35/00; C07K 14/005; C12N 15/86; A61K 2039/5256; A61K 2039/53; C12N 2710/24143; C12N 2770/20034; C12N 2770/20071

(57) ABSTRACT

Designed coronavirus polypeptide sequences are described, and their use as vaccines against viruses of the coronavirus family. The designed sequences include designed coronavirus spike (S) proteins and fragments thereof, including designed full-length S protein sequences SEQ ID NOs: 88, 87, and 53. Designed coronavirus envelope (E), membrane (M), and nucleocapsid (N) protein sequences are also described, and their use as vaccines. Nucleic acid molecules encoding the polypeptides, vectors, fusion proteins, pharmaceutical compositions, cells, and their use as vaccines against viruses of the coronavirus family are also described.

This invention relates to nucleic acid molecules, polypeptides, vectors, cells, fusion proteins, pharmaceutical compositions, combined preparations, and their use as vaccines against viruses of the coronavirus family.

Coronaviruses (CoVs) cause a wide variety of animal and human disease. Notable human diseases caused by CoVs are zoonotic infections, such as severe acute respiratory syndrome (SARS) and Middle-East respiratory syndrome (MERS). Viruses within this family generally cause mild, self-limiting respiratory infections in immunocompetent humans, but can also cause severe, lethal disease characterised by onset of fever, extreme fatigue, breathing difficulties, anoxia, and pneumonia. CoVs transmit through close contact via respiratory droplets of infected subjects, with varying degrees of infectivity within each strain.

CoVs belong to the Coronaviridae family of viruses, all of which are enveloped. CoVs contain a single-stranded positive-sense RNA genome, with a length of between 25 and 31 kilobases (Siddell S. G. 1995, The Coronaviridae), the largest genome so far found in RNA viruses. The Coronaviridae family are subtyped into four genera: α, β, γ, and δ coronaviruses, based on phylogenetic clustering, with each genus subdivided again into clusters depending on the strain of the virus. For example, within the genus β-CoV (Group 2 CoV), four lineages (a, b, c, and d) are commonly recognized:
Lineage A (subgenus Embecovirus) includes HCoV-OC43 and HCoV-HKU1 (various species)
Lineage B (subgenus Sarbecovirus) includes SARSr-COV (which includes all its strains such as SARS-CoV, SARS-CoV-2, and Bat SL-CoV-WIV1)
Lineage C (subgenus Merbecovirus) includes Tylonycteris bat coronavirus HKU4 (BtCoV-HKU4), Pipistrellus bat coronavirus HKU5 (BtCoV-HKU5), and MERS-COV (various species)
Lineage D (subgenus Nobecovirus) includes Rousettus bat coronavirus HKU9 (BtCoV-HKU9)

CoV virions are spherical with characteristic club-shape spike projections emanating from the surface of the virion. The virions contain four main structural proteins: spike(S); membrane (M); envelope (E); and nucleocapsid (N) proteins, all of which are encoded by the viral genome. Some subsets of β-CoVs also comprise a fifth structural protein, hemagglutinin-esterase (HE), which enhances S protein-mediated cell entry and viral spread through the mucosa via its acetyl-esterase activity. Homo-trimers of the S glycoprotein make up the distinctive spike structure on the surface of the virus. These trimers are a class I fusion protein, mediating virus attachment to the host receptor by interaction of the S protein and its receptor. In most CoVs, S is cleaved by host cell protease into two separate polypeptides—S1 and S2. S1 contains the receptor-binding domain (RBD) of the S protein (the exact positioning of the RBD varies depending on the viral strain), while S2 forms the stem of the spike molecule.

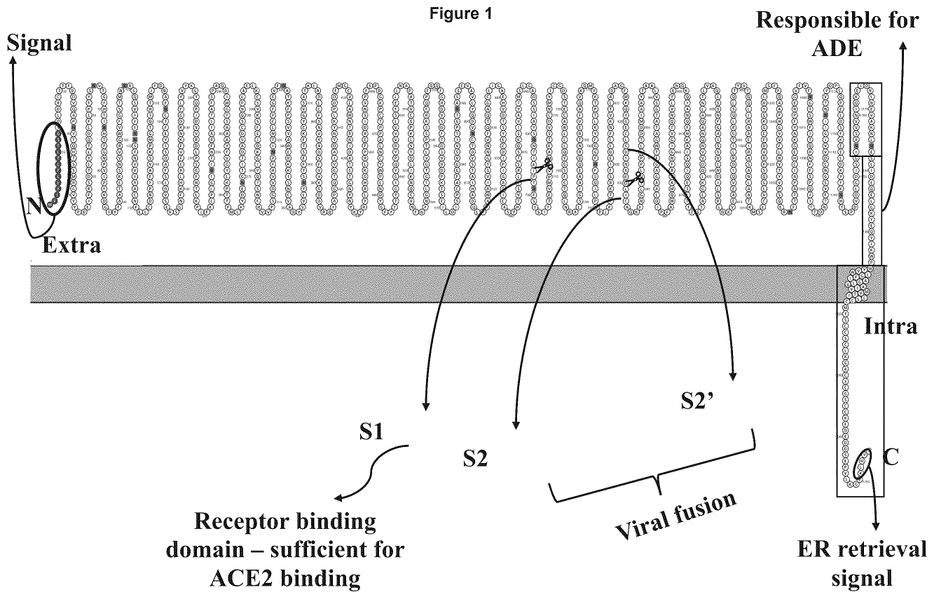

FIG. 1 shows SARS S-protein architecture. The N-terminal sequence is responsible for relaying extracellular signals intracellularly. Studies show that the N-terminal region of the S protein is much more diverse than the C-terminal region, which is highly conserved (Dong et al, Genomic and protein structure modelling analysis depicts the origin and infectivity of 2019-nCoV, a new coronavirus which caused a pneumonia outbreak in Wuhan, China. 2020). The figure shows the S domain, which comprises S1 and S2 domains, responsible for receptor binding and cell membrane fusion respectively.

RNA viruses generally have very high mutation rates compared to DNA viruses, because viral RNA polymerases lack the proofreading ability of DNA polymerases. This is one reason why the virus is able to transmit from its natural host reservoir to other species, and from human to human, and why it is difficult to make effective vaccines to prevent diseases caused by RNA viruses. In most cases, current vaccine candidates against RNA viruses are limited by the viral strain used as the vaccine insert, which is often chosen based on availability of a wild-type strain rather than by informed design. Technical challenges for developing vaccines for enveloped RNA viruses include: i) viral variation of wild-type field isolate glycoproteins (GPS) provide limited breadth of protection as vaccine antigens; ii) selection of vaccine antigens expressed by the vaccine inserts is highly empirical; immunogen selection is a slow, trial and error process; iii) in an evolving or unanticipated viral epidemic, developing new vaccine candidates is time-consuming and can delay vaccine deployment.

Before 2002, CoVs were only thought to cause mild respiratory problems, and were endemic in the human population, causing 15-30% of respiratory tract infections each year. Since their first discovery in the 1960's, the CoV family has expanded massively and has caused many outbreaks in both humans and animals. The SARS pandemic that occurred in 2002-2003 in the Guangdong Province of China was the most severe disease caused by any coronavirus known to that date. During that period, approximately 8098 cases occurred with 774 deaths (mortality rate ˜9.6% overall). The mortality rate was ˜50% in individuals over 90 years of age. The virus, identified as SARS-CoV, a group 2b β-CoV, originated in bats. Two novel virus isolates from bats show more similarity to the human SARS-CoV than any other virus identified to date, and bind to the same cellular receptor as human derived SARS-CoV—angiotensin converting enzyme 2 (ACE2).

While the SARS-CoV epidemic was controlled in 2003, a novel human CoV, a group 2c β-CoV, emerged in the Middle East in 2012. MERS is the causative agent of a series of highly pathogenic respiratory tract infections in the Middle East, with an initial mortality rate of 50%. An estimate of 2,494 cases and 858 deaths caused by MERS has been reported since its emergence, with a total estimated fatality rate by the World Health Organisation (WHO) of 34.4%. Along with SARS-CoV, this novel CoV originated from bats, likely with an intermediate host such as dromedary camels contributing to the spread of the outbreak. This virus utilises dipeptidyl peptidase (DPP4) as its receptor, another peptidase receptor. It is currently unclear why CoVs utilise host peptidases as their binding receptor, as entry occurs even in the absence of enzyme activity.

Towards the end of 2019, another novel CoV emerged; severe acute respiratory syndrome coronavirus 2 (SARS-CoV-2). The outbreak began in Wuhan, China in late 2019. By 30 Jan. 2020 the WHO declared a global health emergency as the virus had spread to over 25 countries within a month of its emergence. The number of SARS-CoV-2 (SARS2) infections increased exponentially across many countries around the world. Efforts to stop the spread of the virus were made, which curtailed the number of cases of infection and the number of deaths caused by the virus. However, second and third waves of the virus have occurred in many countries, resulting (by 22 Apr. 2021, according to the WHO) in global figures of more than 142 million confirmed cases of infection, and over 3 million confirmed deaths.

Since the first described human infection with SARS-CoV-2 in December of 2019, nine vaccines have been approved for use in humans (Craven, 2021, Regulatory Focus, News Articles, 2020, 3, COVID-19 Vaccine Tracker: https://www.raps.org/news-and-articles/news-articles/2020/3/covid-19-vaccine-tracker). As of October 2022, over 37 vaccines have been approved for use in humans, with many more in development (Craven, 2022, Regulatory Focus, News Articles, 2020, 3, COVID-19 Vaccine Tracker: https://www.raps.org/news-and-articles/news-articles/2020/3/covid-19-vaccine-tracker). The AstraZeneca/Oxford COVID-19 vaccine (AZD1222) uses an adenoviral vector. Two of the vaccines currently in use worldwide, BNT162b2 (manufactured by Pfizer) and mRNA-1273 (manufactured by Moderna), are based on lipid nanoparticle delivery of mRNA encoding a prefusion stabilized form of spike protein derived from SARS-CoV-2 isolated early in the epidemic from Wuhan, China. Both of these vaccines demonstrated >94% efficacy at preventing coronavirus disease 2019 (COVID-19) in phase III clinical studies performed in late 2020 in multiple countries (Polack et al., C4591001 Clinical Trial Group (2020). Safety and Efficacy of the BNT162b2 mRNA Covid-19 Vaccine. N. Engl. J. Med. 383, 2603-2615; Baden et al., COVE Study Group (2021). Efficacy and Safety of the mRNA-1273 SARS-CoV-2 Vaccine. N. Engl. J. Med. 384, 403-416). However, the recent emergence of novel circulating variants has raised significant concerns about the effectiveness of the current vaccines, especially in countries such as South Africa and Brazil, where the epidemic is dominated by variant strains (Garcia-Beltran et al., 2021, Cell 184, 2372-2383: Multiple SARS-CoV-2 variants escape neutralization by vaccine-induced humoral immunity).

One of the earliest variants that emerged and rapidly became globally dominant was D614G. In the United Kingdom, a novel lineage termed B.1.1.7 (also known as VOC-202012/01 or 501Y.V1) has rapidly emerged. B.1.1.7 includes three amino acid deletions and seven missense mutations in spike, including D614G as well as N501Y in the ACE2 receptor-binding domain (RBD), and has been reported to be more infectious than D614G. There have also been reports of SARS-CoV-2 transmission between humans and minks in Denmark with a variant called mink cluster 5 or B. 1.1.298, which includes a two-amino acid deletion and four missense mutations including Y453F in RBD. Another variant that recently emerged in California, termed B.1.429, contains four missense mutations in spike, one of which is a single L452R RBD mutation. The ability of B.1.1.298 and B.1.429 variants to evade neutralizing humoral immunity from prior infection or vaccination has yet to be determined. Novel variants arising from the B.1.1.28 lineage first described in Brazil and Japan, termed P.2 (with 3 spike missense mutations) and P.1 (also termed Gamma variant, with 12 spike missense mutations), contain a E484K mutation, and P.1 also contains K417T and N501Y mutations in RBD. These strains have been spreading rapidly, and both P.2 and P. 1 were recently found in documented cases of SARS-CoV-2 reinfection. Of greatest concern has been the emergence of multiple strains of the B.1.351 lineage (also known as 501Y.V2), which were first reported in South Africa and have since spread globally. This lineage contains three RBD mutations, K417N, E484K, and N501Y, in addition to several mutations outside of RBD. B.1.617.2 (Delta variant) then emerged, comprising increased transmissibility. First detected in India in December 2020, the variant contains four mutations in the RBD: L452R, T478K, K417N, and E484K. More recently, the B.1.1.529 (BA. 1/Omicron) variant emerged, comprising 30 mutations in the S protein, 15 of which are in the RBD, which have shown to cause significant humoral immune evasion and high transmissibility. Since then, a number of sub-variants of Omicron have emerged, including BA.2. BA.3, BA.4, and BA.5. Some of these sub-variants also comprise sub-variants, including BA.2.12.1. The emergence of novel variants that appear to escape immune responses has spurred vaccine manufacturers to develop boosters for these spike variants.

Human cases or outbreaks of haemorrhagic fevers caused by coronaviruses occur sporadically and irregularly. The occurrence of outbreaks cannot be easily predicted. With a few exceptions, there is no cure or established drug treatment for CoV infections. Vaccines have only been approved for some CoVs, but these vaccines are not always used because they are either not very effective or in some cases have been reported to promote selection of novel pathogenic CoVs via recombination of circulating strains. By April 2020, several potential vaccines had been developed for SARS-CoV but none had been approved for use. A year later, several novel vaccines have had regulatory approval, and a mass vaccination programme was underway. A year later still, many more vaccines had been granted regulatory approval. The first mass vaccination programme started in early December 2020, and as of 15 Feb. 2021, the WHO estimates that 175.3 million vaccine doses have been administered. At least 7 different vaccines are being used worldwide. WHO issued an Emergency Use Listing (EUL) for the Pfizer-BioNTech COVID-19 vaccine (BNT162b2) on 31 Dec. 2020. On 15 Feb. 2021, WHO issued EULs for two versions of the AstraZeneca/Oxford COVID-19 vaccine (AZD1222). As of 18 Feb. 2021, the UK had administered 12 million people with their first dose of either of the Pfizer-BioNTech or the AstraZeneca/Oxford vaccine. Both the Pfizer and Moderna vaccine use an mRNA platform encoding the S protein. Pfizer uses a nanoparticle vector for nucleic acid delivery, whereas AstraZeneca uses an adenoviral vector.

There are many hurdles to overcome in the development of an effective vaccine for CoVs. Firstly, immunity, whether it is natural or artificial, does not necessarily prevent subsequent infection (Fehr et al. Methods Mol Biol. 2015, 1282:1-23). Secondly, the propensity of the viruses to recombine may pose a problem by rendering the vaccine useless by increasing the genetic diversity of the virus. Additionally, vaccination with the viral S-protein has been shown to lead to enhanced disease in the case of FIPV (feline infectious peritonitis virus), a highly virulent strain of feline CoV. This enhanced pathogenicity of the disease is caused by non-neutralising antibodies that facilitate viral entry into host cells in a process called antibody-dependent enhancement (ADE). After primary infection of one strain of a virus, neutralising antibodies are produced against the same strain of the virus. However, if a different strain infects the host in a secondary infection, non-neutralising antibodies produced during the first infection, which do not neutralise the virus, instead, bind to the virus and then bind to the IgG Fc receptors on immune cells and mediate viral entry into these cells (Wan et al. Journal of Virology. 2020, 94(5):1-13).

When developing vaccines against viruses that are capable of ADE (or of triggering ADE-like pro-inflammatory responses), it is crucial that epitopes are identified that are responsible for eliciting non-neutralising antibodies, and that these epitopes are either masked by modification or are removed from the vaccine. These non-neutralising epitopes on the S-protein may also result in immune diversion wherein the non-neutralising epitopes outcompete neutralising epitopes for binding to antibodies. The neutralising epitopes are neglected by the immune system which fails to neutralise the antigen. In the case of recombinant RBD vaccines, previously buried surfaces containing non-neutralising immunodominant epitopes may become newly exposed which outcompete epitopes responsible for neutralisation by the immune system.

There is a need, therefore, to provide effective vaccines that induce a broadly neutralising immune response to protect against emerging and re-emerging diseases caused by CoVs, especially β-CoVs, such as SARS-CoV and the recent SARS-CoV-2. In particular, there is a need to provide vaccines lacking non-neutralising epitopes that may result in virus immune evasion and disease progression by ADE (or ADE-like pro-inflammatory responses).

There is also a need to provide improved coronavirus vaccines that elicit broadly neutralising antibodies against SARS-CoV-2 variants, in particular against current and recent variants of concern. In particular there is a need to provide effective vaccines that induce a broadly neutralising immune response to protect against the Delta strain and several Omicron strains.

Furthermore, there is a need to provide vaccines that successfully combat vaccine escape of new SARS-CoV-2 variants.

### Designed Coronavirus Spike(s) Protein Sequences (Full-Length, Truncated, and Receptor Binding Domain, RBD)

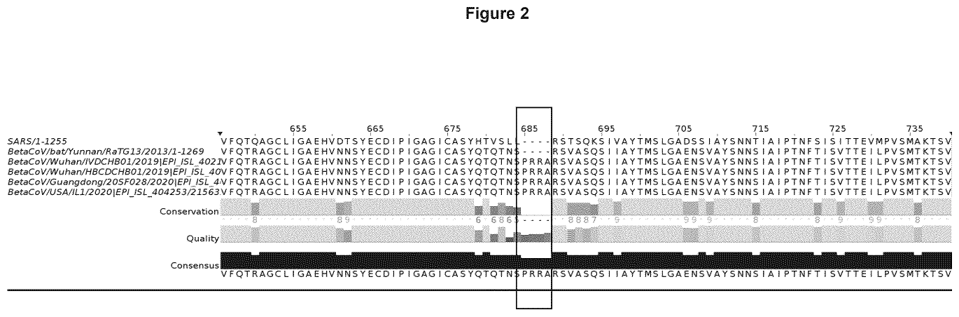

FIG. 2 shows a multiple sequence alignment of the S-protein (the region around the cleavage site 1) comparing SARS-CoV isolate (SARS-CoV-1), and closely related bat betacoronavirus (RaTG13) isolate, with four SARS-CoV-2 isolates. The SARS-CoV S-protein (1269 amino acid residues) shares a high sequence identity (˜73%) with the SARS-CoV-2 S-protein (1273 amino acid residues). Expansion of cleavage site one (shown as a boxed area in the figure) is observed in all SARS-CoV-2 strains so far. The majority of the insertions/substitutions are observed in the subunit 1, with minimal substitutions in the subunit S2, as compared to SARS-CoV-1. The C-terminus contains epitopes which elicit non-neutralising antibodies and are responsible for antibody dependent enhancement.

The applicant has generated a novel amino acid sequence for an S-protein, called CoV_T2_1 (also referred to below as Wuhan-Node-1), which has improved immunogenicity (which allows the protein and its derivatives to elicit a broadly neutralising immune response).

The amino acid sequences of the full length S-protein (SEQ ID NO: 13) (COV_T2_1; Wuhan-Node-1), truncated S-protein (tr, missing the C-terminal part of the S2 sequence) (SEQ ID NO:15) (CoV_T2_4; Wuhan_Node1_tr), and the receptor binding domain (RBD) (SEQ ID NO:17) (CoV_T2_7; Wuhan_Node1_RBD) (and their respective encoding nucleic acid sequences, SEQ ID NOs: 14, 16, 18) are provided in the examples below.

According to the invention there is provided an isolated polypeptide which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17.

According to the invention there is also provided an isolated polypeptide which comprises an amino acid sequence of SEQ ID NO: 17.

SEQ ID NO:17 is the amino acid sequence of a novel S-protein RBD designed by the applicant.

There is also provided according to the invention an isolated polypeptide comprising an amino acid sequence of SEQ ID NO: 15, or an amino acid sequence which has at least 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 15.

According to the invention there is also provided an isolated polypeptide which comprises an amino acid sequence of SEQ ID NO: 15.

There is also provided according to the invention an isolated polypeptide comprising an amino acid sequence of SEQ ID NO: 13, or an amino acid sequence which has at least 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 13.

According to the invention there is also provided an isolated polypeptide which comprises an amino acid sequence of SEQ ID NO: 13.

Examples 6 and 7 below provide amino acid sequence alignments of the novel S-protein RBD amino acid sequence (Wuhan_Node1_RBD (COV_T2_7) (SEQ ID NO:17)) with the RBD amino acid sequences of SARS-TOR2 isolate AY274119 (AY274119_RBD (COV_T2_5) (SEQ ID NO: 5)), and SARS_COV_2 isolate hCov-19/Wuhan/LVDC-HB-01/2019 (EPI_ISL_402119) (EPI_ISL_402119_RBD (COV_T2_6) (SEQ ID NO:11)), respectively.

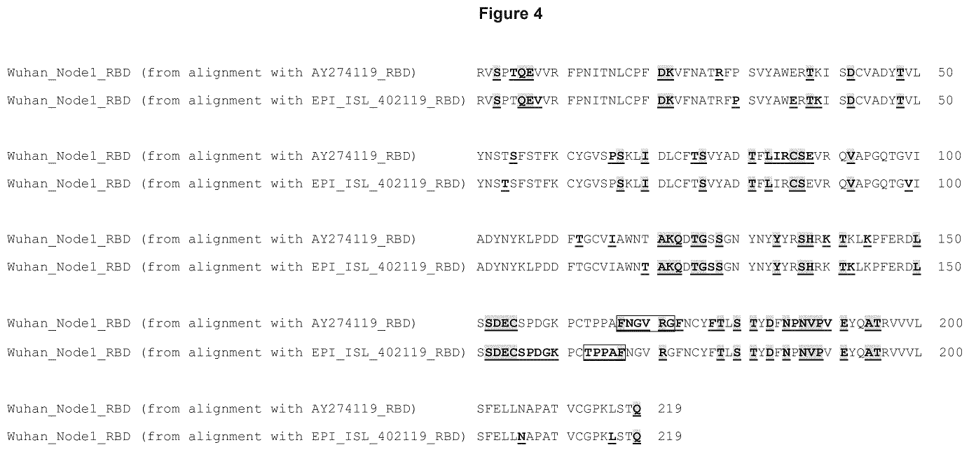

As explained in Example 9 below, FIG. 4 shows Wuhan_Node1_RBD (CoV_T2_7) amino acid sequence (SEQ ID NO:17) with amino acid residue differences highlighted in bold and underline from the respective alignments with AY274119_RBD (COV_T2_5) (SEQ ID NO:5) and EPI_ISL_402119_RBD (COV_T2_6) (SEQ ID NO:11) amino acid sequences (Examples 6 and 7, respectively). The amino acid residue differences from the two alignments are listed in the table below (the numbering of residue positions corresponds to positions of the Wuhan_Node1_RBD (COV_T2_7) (SEQ ID NO:17) amino acid sequence. The common differences from the two alignments are at amino acid residues: 3, 6, 7, 21, 22, 38, 42, 48, 67, 70, 76, 81, 83, 86, 87, 92, 121, 122, 123, 125, 126, 128, 134, 137, 138, 141, 150, 152, 153, 154, 155, 167, 171, 178, 180, 181, 183, 185, 187, 188, 189, 191, 194, 195, 219 (shown with grey highlighting in FIG. 4, and in the table below):

TABLE 1

| Wuhan_Node1_RBD | Amino acid residue | Amino acid residue |
|---|---|---|
| (CoV_T2_7) residue | difference vs | difference vs |
| position | AY274119_RBD | EPI_ISL_402119_RBD |
| 3 | S | S |
| 5 | T | — |
| 6 | Q | Q |
| 7 | E | E |
| 8 | — | V |
| 21 | D | D |
| 22 | K | K |
| 28 | R | — |
| 30 | — | P |
| 36 | — | E |
| 38 | T | T |
| 39 | — | K |
| 42 | D | D |
| 48 | T | T |
| 54 | — | T |
| 55 | S | — |
| 66 | P | — |
| 67 | S | S |
| 70 | I | I |
| 75 | T | — |
| 76 | S | S |
| 81 | T | T |
| 83 | L | L |
| 84 | I | — |
| 85 | R | — |
| 86 | C | C |
| 87 | S | S |
| 88 | E | — |
| 92 | V | V |
| 99 | — | V |
| 112 | T | — |
| 116 | I | — |
| 120 | — | T |
| 121 | A | A |
| 122 | K | K |
| 123 | Q | Q |
| 125 | T | T |
| 126 | G | G |
| 127 | — | S |
| 128 | S | S |
| 134 | Y | Y |
| 137 | S | S |
| 138 | H | H |
| 140 | K | — |
| 141 | T | T |
| 142 | — | K |
| 144 | K | — |
| 150 | L | L |
| 152 | S | S |
| 153 | D | D |
| 154 | E | E |
| 155 | C | C |
| 156 | — | S |
| 157 | — | P |
| 158 | — | D |
| 159 | — | G |
| 160 | — | K |
| 163 | — | T |
| 164 | — | P |
| 165 | — | P |
| 166 | — | A |
| 167 | F | F |
| 168 | N | — |
| 169 | G | — |
| 170 | V | — |
| 171 | R | R |
| 172 | G | — |
| 173 | F | — |
| 177 | F | — |
| 178 | T | T |
| 180 | S | S |
| 181 | T | T |
| 183 | D | D |
| 185 | N | N |
| 186 | P | — |
| 187 | N | N |
| 188 | V | V |
| 189 | P | P |
| 190 | V | — |
| 191 | E | E |
| 194 | A | A |
| 195 | T | T |
| 206 | — | N |
| 216 | — | L |
| 219 | Q | Q |

Amino acid insertions are at positions 167-172 (compared to AY274119_RBD), and 163-167 (compared to EPI_ISL_402119_RBD) (shown boxed in FIG. 4).

Optionally an isolated polypeptide of the invention comprises at least one of the amino acid residues, at a position corresponding to the amino acid residue position of SEQ ID NO: 17, as shown in Table 2 below:

TABLE 2

| Wuhan_Node1_RBD | Amino acid |
|---|---|
| (CoV_T2_7) residue position | residue |
| 3 | S |
| 6 | Q |
| 7 | E |
| 21 | D |
| 22 | K |
| 38 | T |
| 42 | D |
| 48 | T |
| 67 | S |
| 70 | I |
| 76 | S |
| 81 | T |
| 83 | L |
| 86 | C |
| 87 | S |
| 92 | V |
| 121 | A |
| 122 | K |
| 123 | Q |
| 125 | T |
| 126 | G |
| 128 | S |
| 134 | Y |
| 137 | S |
| 138 | H |
| 141 | T |
| 150 | L |
| 152 | S |
| 153 | D |
| 154 | E |
| 155 | C |
| 167 | F |
| 171 | R |
| 178 | T |
| 180 | S |
| 181 | T |
| 183 | D |
| 185 | N |
| 187 | N |
| 188 | V |
| 189 | P |
| 191 | E |
| 194 | A |
| 195 | T |
| 219 | Q |

Optionally an isolated polypeptide of the invention comprises at least five of the amino acid residues, at positions corresponding to the amino acid residue positions of SEQ ID NO: 17, as shown in Table 2.

Optionally an isolated polypeptide of the invention comprises at least ten of the amino acid residues, at positions corresponding to the amino acid residue positions of SEQ ID NO: 17, as shown in Table 2.

Optionally an isolated polypeptide of the invention comprises at least fifteen of the amino acid residues, at positions corresponding to the amino acid residue positions of SEQ ID NO: 17, as shown in Table 2.

Optionally an isolated polypeptide of the invention comprises at least twenty of the amino acid residues, at positions corresponding to the amino acid residue positions of SEQ ID NO: 17, as shown in Table 2.

Optionally an isolated polypeptide of the invention comprises at least twenty five of the amino acid residues, at positions corresponding to the amino acid residue positions of SEQ ID NO: 17, as shown in Table 2.

Optionally an isolated polypeptide of the invention comprises at least thirty of the amino acid residues, at positions corresponding to the amino acid residue positions of SEQ ID NO: 17, as shown in Table 2.

Optionally an isolated polypeptide of the invention comprises at least thirty five of the amino acid residues, at positions corresponding to the amino acid residue positions of SEQ ID NO: 17, as shown in Table 2.

Optionally an isolated polypeptide of the invention comprises at least forty of the amino acid residues, at positions corresponding to the amino acid residue positions of SEQ ID NO: 17, as shown in Table 2.

Optionally an isolated polypeptide of the invention comprises all of the amino acid residues, at positions corresponding to the amino acid residue positions of SEQ ID NO:17, as shown in Table 2.

Optionally an isolated polypeptide of the invention comprises at least one of the amino acid residues, at a position corresponding to the amino acid residue position of SEQ ID NO: 17, as shown in Table 3 below:

TABLE 3

| Wuhan_Node1_RBD | Amino acid |
|---|---|
| (CoV_T2_7) residue position | residue |
| 3 | S |
| 6 | Q |
| 7 | E |
| 8 | V |
| 21 | D |
| 22 | K |
| 30 | P |
| 36 | E |
| 38 | T |
| 39 | K |
| 42 | D |
| 48 | T |
| 54 | T |
| 67 | S |
| 70 | I |
| 76 | S |
| 81 | T |
| 83 | L |
| 86 | C |
| 87 | S |
| 92 | V |
| 99 | V |
| 120 | T |
| 121 | A |
| 122 | K |
| 123 | Q |
| 125 | T |
| 126 | G |
| 127 | S |
| 128 | S |
| 134 | Y |
| 137 | S |
| 138 | H |
| 141 | T |
| 142 | K |
| 150 | L |
| 152 | S |
| 153 | D |
| 154 | E |
| 155 | C |
| 156 | S |
| 157 | P |
| 158 | D |
| 159 | G |
| 160 | K |
| 163 | T |
| 164 | P |
| 165 | P |
| 166 | A |
| 167 | F |
| 171 | R |
| 178 | T |
| 180 | S |
| 181 | T |
| 183 | D |
| 185 | N |
| 187 | N |
| 188 | V |
| 189 | P |
| 191 | E |
| 194 | A |
| 195 | T |
| 206 | N |
| 216 | L |
| 219 | Q |

Optionally an isolated polypeptide of the invention comprises at least five of the amino acid residues, at positions corresponding to the amino acid residue positions of SEQ ID NO: 17, as shown in Table 3.

Optionally an isolated polypeptide of the invention comprises at least ten of the amino acid residues, at positions corresponding to the amino acid residue positions of SEQ ID NO: 17, as shown in Table 3.

Optionally an isolated polypeptide of the invention comprises at least fifteen of the amino acid residues, at positions corresponding to the amino acid residue positions of SEQ ID NO: 17, as shown in Table 3.

Optionally an isolated polypeptide of the invention comprises at least twenty of the amino acid residues, at positions corresponding to the amino acid residue positions of SEQ ID NO: 17, as shown in Table 3.

Optionally an isolated polypeptide of the invention comprises at least twenty five of the amino acid residues, at positions corresponding to the amino acid residue positions of SEQ ID NO: 17, as shown in Table 3.

Optionally an isolated polypeptide of the invention comprises at least thirty of the amino acid residues, at positions corresponding to the amino acid residue positions of SEQ ID NO: 17, as shown in Table 3.

Optionally an isolated polypeptide of the invention comprises at least thirty five of the amino acid residues, at positions corresponding to the amino acid residue positions of SEQ ID NO: 17, as shown in Table 3.

Optionally an isolated polypeptide of the invention comprises at least forty of the amino acid residues, at positions corresponding to the amino acid residue positions of SEQ ID NO: 17, as shown in Table 3.

Optionally an isolated polypeptide of the invention comprises at least forty five of the amino acid residues, at positions corresponding to the amino acid residue positions of SEQ ID NO: 17, as shown in Table 3.

Optionally an isolated polypeptide of the invention comprises at least fifty of the amino acid residues, at positions corresponding to the amino acid residue positions of SEQ ID NO: 17, as shown in Table 3.

Optionally an isolated polypeptide of the invention comprises at least fifty five of the amino acid residues, at positions corresponding to the amino acid residue positions of SEQ ID NO: 17, as shown in Table 3.

Optionally an isolated polypeptide of the invention comprises at least sixty of the amino acid residues, at positions corresponding to the amino acid residue positions of SEQ ID NO: 17, as shown in Table 3.

Optionally an isolated polypeptide of the invention comprises all of the amino acid residues, at positions corresponding to the amino acid residue positions of SEQ ID NO: 17, as shown in Table 3.

Optionally an isolated polypeptide of the invention comprises at least one of the amino acid residues, at a position corresponding to the amino acid residue position of SEQ ID NO: 17, as shown in Table 4 below:

TABLE 4

| Wuhan_Node1_RBD | Amino acid |
|---|---|
| (CoV_T2_7) residue position | residue |
| 3 | S |
| 5 | T |
| 6 | Q |
| 7 | E |
| 21 | D |
| 22 | K |
| 28 | R |
| 38 | T |
| 42 | D |
| 48 | T |
| 55 | S |
| 66 | P |
| 67 | S |
| 70 | I |
| 75 | T |
| 76 | S |
| 81 | T |
| 83 | L |
| 84 | I |
| 85 | R |
| 86 | C |
| 87 | S |
| 88 | E |
| 92 | V |
| 112 | T |
| 116 | I |
| 121 | A |
| 122 | K |
| 123 | Q |
| 125 | T |
| 126 | G |
| 128 | S |
| 134 | Y |
| 137 | S |
| 138 | H |
| 140 | K |
| 141 | T |
| 144 | K |
| 150 | L |
| 152 | S |
| 153 | D |
| 154 | E |
| 155 | C |
| 167 | F |
| 168 | N |
| 169 | G |
| 170 | V |
| 171 | R |
| 172 | G |
| 173 | F |
| 177 | F |
| 178 | T |
| 180 | S |
| 181 | T |
| 183 | D |
| 185 | N |
| 186 | P |
| 187 | N |
| 188 | V |
| 189 | P |
| 190 | V |
| 191 | E |
| 194 | A |
| 195 | T |
| 219 | Q |

Optionally an isolated polypeptide of the invention comprises at least five of the amino acid residues, at positions corresponding to the amino acid residue positions of SEQ ID NO: 17, as shown in Table 4.

Optionally an isolated polypeptide of the invention comprises at least ten of the amino acid residues, at positions corresponding to the amino acid residue positions of SEQ ID NO: 17, as shown in Table 4.

Optionally an isolated polypeptide of the invention comprises at least fifteen of the amino acid residues, at positions corresponding to the amino acid residue positions of SEQ ID NO: 17, as shown in Table 4.

Optionally an isolated polypeptide of the invention comprises at least twenty of the amino acid residues, at positions corresponding to the amino acid residue positions of SEQ ID NO: 17, as shown in Table 4.

Optionally an isolated polypeptide of the invention comprises at least twenty five of the amino acid residues, at positions corresponding to the amino acid residue positions of SEQ ID NO: 17, as shown in Table 4.

Optionally an isolated polypeptide of the invention comprises at least thirty of the amino acid residues, at positions corresponding to the amino acid residue positions of SEQ ID NO: 17, as shown in Table 4.

Optionally an isolated polypeptide of the invention comprises at least thirty five of the amino acid residues, at positions corresponding to the amino acid residue positions of SEQ ID NO: 17, as shown in Table 4.

Optionally an isolated polypeptide of the invention comprises at least forty of the amino acid residues, at positions corresponding to the amino acid residue positions of SEQ ID NO: 17, as shown in Table 4.

Optionally an isolated polypeptide of the invention comprises at least forty five of the amino acid residues, at positions corresponding to the amino acid residue positions of SEQ ID NO: 17, as shown in Table 4.

Optionally an isolated polypeptide of the invention comprises at least fifty of the amino acid residues, at positions corresponding to the amino acid residue positions of SEQ ID NO: 17, as shown in Table 4.

Optionally an isolated polypeptide of the invention comprises at least fifty five of the amino acid residues, at positions corresponding to the amino acid residue positions of SEQ ID NO: 17, as shown in Table 4.

Optionally an isolated polypeptide of the invention comprises at least sixty of the amino acid residues, at positions corresponding to the amino acid residue positions of SEQ ID NO: 17, as shown in Table 4.

Optionally an isolated polypeptide of the invention comprises all of the amino acid residues, at positions corresponding to the amino acid residue positions of SEQ ID NO: 17, as shown in Table 4.

According to the invention there is also provided an isolated polypeptide, which comprises a coronavirus S protein RBD domain with any, or all of the amino acid residues at positions corresponding to the amino acid residue positions as shown in Table 5 below:

TABLE 5

| S protein RBD | Amino acid |
|---|---|
| residue position | residue |
| 3 | S |
| 6 | Q |
| 7 | E |
| 21 | D |
| 22 | K |
| 38 | T |
| 42 | D |
| 48 | T |
| 67 | S |
| 70 | I |
| 76 | S |
| 81 | T |
| 83 | L |
| 86 | C |
| 87 | S |
| 92 | V |
| 121 | A |
| 122 | K |
| 123 | Q |
| 125 | T |
| 126 | G |
| 128 | S |
| 134 | Y |
| 137 | S |
| 138 | H |
| 141 | T |
| 150 | L |
| 152 | S |
| 153 | D |
| 154 | E |
| 155 | C |
| 167 | F |
| 171 | R |
| 178 | T |
| 180 | S |
| 181 | T |
| 183 | D |
| 185 | N |
| 187 | N |
| 188 | V |
| 189 | P |
| 191 | E |
| 194 | A |
| 195 | T |
| 219 | Q |

According to the invention there is also provided an isolated polypeptide, which comprises a coronavirus S protein RBD domain with any, or all of the amino acid residues at positions corresponding to the amino acid residue positions as shown in Table 6 below:

TABLE 6

| S protein RBD | Amino acid |
|---|---|
| residue position | residue |
| 3 | S |
| 6 | Q |
| 7 | E |
| 8 | V |
| 21 | D |
| 22 | K |
| 30 | P |
| 36 | E |
| 38 | T |
| 39 | K |
| 42 | D |
| 48 | T |
| 54 | T |
| 67 | S |
| 70 | I |
| 76 | S |
| 81 | T |
| 83 | L |
| 86 | C |
| 87 | S |
| 92 | V |
| 99 | V |
| 120 | T |
| 121 | A |
| 122 | K |
| 123 | Q |
| 125 | T |
| 126 | G |
| 127 | S |
| 128 | S |
| 134 | Y |
| 137 | S |
| 138 | H |
| 141 | T |
| 142 | K |
| 150 | L |
| 152 | S |
| 153 | D |
| 154 | E |
| 155 | C |
| 156 | S |
| 157 | P |
| 158 | D |
| 159 | G |
| 160 | K |
| 163 | T |
| 164 | P |
| 165 | P |
| 166 | A |
| 167 | F |
| 171 | R |
| 178 | T |
| 180 | S |
| 181 | T |
| 183 | D |
| 185 | N |
| 187 | N |
| 188 | V |
| 189 | P |
| 191 | E |
| 194 | A |
| 195 | T |
| 206 | N |
| 216 | L |
| 219 | Q |

There is also provided according to the invention an isolated polypeptide, which comprises a coronavirus S protein RBD domain with any, or all of the amino acid residues at positions corresponding to the amino acid residue positions as shown in Table 7 below:

TABLE 7

| S protein RBD | Amino acid |
|---|---|
| residue position | residue |
| 3 | S |
| 5 | T |
| 6 | Q |
| 7 | E |
| 21 | D |
| 22 | K |
| 28 | R |
| 38 | T |
| 42 | D |
| 48 | T |
| 55 | S |
| 66 | P |
| 67 | S |
| 70 | I |
| 75 | T |
| 76 | S |
| 81 | T |
| 83 | L |
| 84 | I |
| 85 | R |
| 86 | C |
| 87 | S |
| 88 | E |
| 92 | V |
| 112 | T |
| 116 | I |
| 121 | A |
| 122 | K |
| 123 | Q |
| 125 | T |
| 126 | G |
| 128 | S |
| 134 | Y |
| 137 | S |
| 138 | H |
| 140 | K |
| 141 | T |
| 144 | K |
| 150 | L |
| 152 | S |
| 153 | D |
| 154 | E |
| 155 | C |
| 167 | F |
| 168 | N |
| 169 | G |
| 170 | V |
| 171 | R |
| 172 | G |
| 173 | F |
| 177 | F |
| 178 | T |
| 180 | S |
| 181 | T |
| 183 | D |
| 185 | N |
| 186 | P |
| 187 | N |
| 188 | V |
| 189 | P |
| 190 | V |
| 191 | E |
| 194 | A |
| 195 | T |
| 219 | Q |

Optionally an isolated polypeptide of the invention which comprises a coronavirus S protein RBD domain comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:5.

Optionally an isolated polypeptide of the invention which comprises a coronavirus S protein RBD domain comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:11.

Further novel S protein RBD sequences are referred to herein as COV_S_T2_13-COV_S_T2_18 (SEQ ID NOs: 27-32, respectively). CoV_S_T2_13 is the direct output of our design algorithm, and CoV_S_T2_14-COV_S_T2_18 are epitope-enriched versions of COV_S_T2_13. The amino acid sequences of these designed sequences are provided below, and in Example 12:

>COV_S_T2_13
(SEQ ID NO: 27)
RVAPTKEVVR FPNITNLCPF GEVFNATRFP SVYAWERKRI SNCVADYSVL YNSTSFSTFK CYGVSPTKLN
DLCFTNVYAD SFVIRGDEVR QIAPGQTGVI ADYNYKLPDD FTGCVIAWNT NNLDSTTGGN YNYLYRSLRK
SKLKPFERDI SSDIYSPGGK PCSGVEGFNC YYPLRSYGFF PTNGVGYQPY RVVVLSFELL NAPATVCGPK
LSTD
>COV_S_T2_14
(SEQ ID NO: 28)
RVAPTKEVVR FPNITNLCPF GEVFNATKFP SVYAWERKKI SNCVADYSVL YNSTSFSTFK CYGVSPTKLN
DLCFTNVYAD SFVIRGDEVR QIAPGQTGVI ADYNYKLPDD FTGCVIAWNT NNIDSTTGGN YNYLYRSLRK
SKLKPFERDI SSDIYSPGGK PCSGVEGFNC YYPLRSYGFF PTNGVGYQPY RVVVLSFELL NAPATVCGPK
LSTD
>COV_S_T2_15
(SEQ ID NO: 29)
RVAPTKEVVR FPNITNLCPF GEVFNATRFP SVYAWERKRI SNCVADYSVL YNSTFFSTFK CYGVSPTKLN
DLCFSNVYAD SFVIRGDEVR QIAPGQTGVI ADYNYKLPDD FMGCVIAWNT NNLDSTTGGN YNYLYRSLRK
SKLKPFERDI SSDIYSPGGK PCSGVEGFNC YYPLRSYGFF PTNGVGYQPY RVVVLSFELL NAPATVCGPK
LSTD
>COV_S_T2_16
(SEQ ID NO: 30)
RVAPTKEVVR FPNITNLCPF GEVFNATRFP SVYAWERKRI SNCVADYSVL YNSTSFSTFK CYGVSPTKLN
DLCFTNVYAD SFVIRGDEVR QIAPGQTGKI ADYNYKLPDD FTGCVIAWNT NNLDSTTGGN YNYLYRLFRK
SNLKPFERDI SSDIYQAGST PCSGVEGFNC YFPLQSYGFQ PTNGVGYQPY RVVVLSFELL NAPATVCGPK
LSTD
>COV_S_T2_17
(SEQ ID NO: 31)
RVAPTKEVVR FPNITNLCPF GEVFNATKEP SVYAWERKKI SNCVADYSVL YNSTSFSTFK CYGVSPTKLN
DLCFTNVYAD SFVIRGDEVR QIAPGQTGVI ADYNYKLPDD FTGCVIAWNT NNIDSTTGGN YNYLYRSLRK
SKLKPFERDI SSDIYSPGGK PCSGVEGFNC YYPLRSYGFF PTNGTGYQPY RVVVLSFELL NAPATVCGPK
LSTD
>COV_S_T2_18
(SEQ ID NO: 32)
RVAPTKEVVR FPNITNLCPF GEVFNATRFP SVYAWERKRI SNCVADYSVL YNSTFFSTFK CYGVSPTKLN
DLCFSNVYAD SFVIRGDEVR QIAPGQTGVI ADYNYKLPDD FMGCVIAWNT NNLDSTTGGN YNYLYRSLRK
SKLKPFERDI SSDIYSPGGK PCSGVEGFNC YYPLRSYGFF PTNGTGYQPY RVVVLSFELL NAPATVCGPK
LSTD

Alignment of these sequences with SARS2 Reference sequence (EPI_ISL_402119_RBD (COV_T2_6) (SEQ ID NO:11)) is shown in Example 12 below.

The amino acid differences of the designed sequences from the SARS2 reference sequence are shown in Table 8.1 below (with differences from the reference sequence highlighted in bold, and differences that are common to all the designed sequences underlined):

TABLE 8.1

| SARS2 RBD | | T2_13 | T2_14 | T2_15 | T2_16 | T2_17 | T2_18 |
|---|---|---|---|---|---|---|---|
| (CoV_T2_6; SEQ | | residue | residue | residue | residue | residue | residue |
| ID NO: 11) | Reference | (SEQ ID | (SEQ ID | (SEQ ID | (SEQ ID | (SEQ ID | (SEQ ID |
| residue position | residue | NO: 27) | NO: 28) | NO: 29) | NO: 30) | NO: 31) | NO: 32) |
| 3 | Q | A | A | A | A | A | A |
| 6 | E | K | K | K | K | K | K |
| 7 | S | E | E | E | E | E | E |
| 8 | I | V | V | V | V | V | V |
| 28 | R | R | K | R | R | K | R |
| 30 | A | P | P | P | P | P | P |
| 36 | N | E | E | E | E | E | E |
| 39 | R | R | K | R | R | K | R |
| 54 | A | T | T | T | T | T | T |
| 55 | S | S | S | F | S | S | F |
| 75 | T | T | T | S | T | T | S |
| 99 | K | V | V | V | K | V | V |
| 112 | T | T | T | M | T | T | M |
| 120 | S | T | T | T | T | T | T |
| 123 | L | L | I | L | L | I | L |
| 126 | K | T | T | T | T | T | T |
| 127 | V | T | T | T | T | T | T |
| 137 | L | S | S | S | L | S | S |
| 138 | F | L | L | L | F | L | L |
| 142 | N | K | K | K | N | K | K |
| 152 | T | S | S | S | S | S | S |
| 153 | E | D | D | D | D | D | D |
| 156 | Q | S | S | S | Q | S | S |
| 157 | A | P | P | P | A | P | P |
| 159 | S | G | G | G | S | G | G |
| 160 | T | K | K | K | T | K | K |
| 163 | N | S | S | S | S | S | S |
| 172 | F | Y | Y | Y | F | Y | Y |
| 175 | Q | R | R | R | Q | R | R |
| 180 | Q | F | F | F | Q | F | F |
| 185 | V | V | V | V | V | T | T |
| 201 | H | N | N | N | N | N | N |
| 211 | K | L | L | L | L | L | L |
| 214 | N | D | D | D | D | D | D |
| Total no of | — | 27 | 30 | 30 | 16 | 31 | 31 |
| differences from | | | | | | | |
| reference | | | | | | | |
| Percentage | — | 87.38 | 85.98 | 85.98 | 92.52 | 85.51 | 85.51 |
| identity with | | | | | | | |
| reference | | | | | | | |

The amino acid changes common to all of the designed sequences are summarised in Table 8.2 below:

TABLE 8.2

| SARS2 RBD (CoV_T2_6; | | |
|---|---|---|
| SEQ ID NO: 11) | Reference | Design |
| residue position | residue | residue |
| 3 | Q | A |
| 6 | E | K |
| 7 | S | E |
| 8 | I | V |
| 30 | A | P |
| 36 | N | E |
| 54 | A | T |
| 120 | S | T |
| 126 | K | T |
| 127 | V | T |
| 152 | T | S |
| 153 | E | D |
| 163 | N | S |
| 201 | H | N |
| 211 | K | L |
| 214 | N | D |

Optional additional changes are summarised in Table 8.3 below:

TABLE 8.3

| SARS2 RBD (CoV_T2_6; | | |
|---|---|---|
| SEQ ID NO: 11) | Reference | Design |
| residue position | residue | residue |
| 99 | K | V |
| 137 | L | S |
| 138 | F | L |
| 142 | N | K |
| 156 | Q | S |
| 157 | A | P |
| 159 | S | G |
| 160 | T | K |
| 172 | F | Y |
| 175 | Q | R |
| 180 | Q | F |

The additional changes listed in Table 8.3 are found in SEQ ID NOs: 27-29, 31, and 32.

Further optional additional changes are summarised in Tables 8.4-8.6 below:

TABLE 8.4

| SARS2 RBD (CoV_T2_6; | | | |
|---|---|---|---|
| SEQ ID NO: 11) | Reference | Design | Found in |
| residue position | residue | residue | SEQ ID NO: |
| 28 | R | K | 28, 31 |
| 39 | R | K | 28, 31 |
| 123 | L | I | 28, 31 |

TABLE 8.5

| SARS2 RBD (CoV_T2_6; | | | |
|---|---|---|---|
| SEQ ID NO: 11) | Reference | Design | Found in |
| residue position | residue | residue | SEQ ID NO: |
| 55 | S | F | 29, 32 |
| 75 | T | S | 29, 32 |
| 112 | T | M | 29, 32 |

TABLE 8.6

| SARS2 RBD (CoV_T2_6; | | | |
|---|---|---|---|
| SEQ ID NO: 11) | Reference | Design | Found in |
| residue position | residue | residue | SEQ ID NO: |
| 185 | V | T | 31, 32 |

According to the invention there is also provided an isolated polypeptide comprising an amino acid sequence of SEQ ID NO: 27 (COV_S_T2_13), or an amino acid sequence which has at least 88%, 89%, 90%, 91%, 92%, 93%, 94%, 95%, 96%, 97%, 98%, or 99% amino acid identity over its entire length with the amino acid sequence of SEQ ID NO:27.

According to the invention there is also provided an isolated polypeptide comprising an amino acid sequence of SEQ ID NO: 28 (COV_S_T2_14), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28.

According to the invention there is also provided an isolated polypeptide comprising an amino acid sequence of SEQ ID NO: 29 (COV_S_T2_15), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29.

According to the invention there is also provided an isolated polypeptide comprising an amino acid sequence of SEQ ID NO: 30 (COV_S_T2_16), or an amino acid sequence which has at least 93%, 94%, 95%, 96%, 97%, 98%, or 99% amino acid identity over its entire length with the amino acid sequence of SEQ ID NO:30.

According to the invention there is also provided an isolated polypeptide comprising an amino acid sequence of SEQ ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31.

According to the invention there is also provided an isolated polypeptide comprising an amino acid sequence of SEQ ID NO: 32 (COV_S_T2_18), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32.

Optionally a polypeptide of the invention comprising an amino acid sequence of SEQ ID NO:27 (COV_S_T2_13), or an amino acid sequence which has at least 88%, 89%, 90%, 91%, 92%, 93%, 94%, 95%, 96%, 97%, 98%, or 99% amino acid identity over its entire length with the amino acid sequence of SEQ ID NO:27, comprises at least one, or all of the amino acid residues at positions corresponding to the amino acid residue positions of SEQ ID NO: 11 as shown in Table 8.2 above.

Optionally a polypeptide of the invention comprising an amino acid sequence of SEQ ID NO: 28 (COV_S_T2_14), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28, comprises at least one, or all of the amino acid residues at positions corresponding to the amino acid residue positions as shown in Table 8.2 above.

Optionally a polypeptide of the invention comprising an isolated polypeptide comprising an amino acid sequence of SEQ ID NO: 29 (COV_S_T2_15), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29, comprises at least one, or all of the amino acid residues at positions corresponding to the amino acid residue positions as shown in Table 8.2 above.

Optionally a polypeptide of the invention comprising an amino acid sequence of SEQ ID NO: 30 (COV_S_T2_16), or an amino acid sequence which has at least 93%, 94%, 95%, 96%, 97%, 98%, or 99% amino acid identity over its entire length with the amino acid sequence of SEQ ID NO:30, comprises at least one, or all of the amino acid residues at positions corresponding to the amino acid residue positions as shown in Table 8.2 above.

Optionally a polypeptide of the invention comprising an amino acid sequence of SEQ ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31, comprises at least one, or all of the amino acid residues at positions corresponding to the amino acid residue positions as shown in Table 8.2 above.

Optionally a polypeptide of the invention comprising an amino acid sequence of SEQ ID NO: 32 (COV_S_T2_18), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32, comprises at least one, or all of the amino acid residues at positions corresponding to the amino acid residue positions as shown in Table 8.2 above.

Optionally a polypeptide of the invention comprising an amino acid sequence of SEQ ID NO:27 (COV_S_T2_13), or an amino acid sequence which has at least 88%, 89%, 90%, 91%, 92%, 93%, 94%, 95%, 96%, 97%, 98%, or 99% amino acid identity over its entire length with the amino acid sequence of SEQ ID NO:27, further comprises at least one, or all of the amino acid residues at positions corresponding to the amino acid residue positions as shown in Table 8.3 above.

Optionally a polypeptide of the invention comprising an amino acid sequence of SEQ ID NO: 28 (COV_S_T2_14), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28, further comprises at least one, or all of the amino acid residues at positions corresponding to the amino acid residue positions as shown in Table 8.3 above.

Optionally a polypeptide of the invention comprising an amino acid sequence of SEQ ID NO: 29 (COV_S_T2_15), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29, further comprises at least one, or all of the amino acid residues at positions corresponding to the amino acid residue positions as shown in Table 8.3 above.

Optionally a polypeptide of the invention comprising an amino acid sequence of SEQ ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31, further comprises at least one, or all of the amino acid residues at positions corresponding to the amino acid residue positions as shown in Table 8.3 above.

Optionally a polypeptide of the invention comprising an amino acid sequence of SEQ ID NO: 32 (COV_S_T2_18), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32, further comprises at least one, or all of the amino acid residues at positions corresponding to the amino acid residue positions as shown in Table 8.3 above. Optionally a polypeptide of the invention comprising an amino acid sequence of SEQ ID NO: 28 (COV_S_T2_14), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28, further comprises at least one, or all of the amino acid residues at positions corresponding to the amino acid residue positions as shown in Table 8.4 above.

Optionally a polypeptide of the invention comprising an isolated polypeptide comprising an amino acid sequence of SEQ ID NO: 29 (COV_S_T2_15), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29, further comprises at least one, or all of the amino acid residues at positions corresponding to the amino acid residue positions as shown in Table 8.5 above.

Optionally a polypeptide of the invention comprising an amino acid sequence of SEQ ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31, further comprises at least one, or all of the amino acid residues at positions corresponding to the amino acid residue positions as shown in Table 8.4 above.

Optionally a polypeptide of the invention comprising an amino acid sequence of SEQ ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31, further comprises at least one, or all of the amino acid residues at positions corresponding to the amino acid residue positions as shown in Table 8.6 above.

Optionally a polypeptide of the invention comprising an amino acid sequence of SEQ ID NO: 32 (COV_S_T2_18), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32, further comprises at least one, or all of the amino acid residues at positions corresponding to the amino acid residue positions as shown in Table 8.5 above.

Optionally a polypeptide of the invention comprising an amino acid sequence of SEQ ID NO: 32 (COV_S_T2_18), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32, further comprises at least one, or all of the amino acid residues at positions corresponding to the amino acid residue positions as shown in Table 8.6 above.

According to the invention there is provided an isolated polypeptide, which comprises an amino acid sequence of SEQ ID NO: 27 (COV_S_T2_13).

According to the invention there is provided an isolated polypeptide, which comprises an amino acid sequence of SEQ ID NO:28 (COV_S_T2_14).

According to the invention there is provided an isolated polypeptide, which comprises an amino acid sequence of SEQ ID NO:29 (COV_S_T2_15).

According to the invention there is provided an isolated polypeptide, which comprises an amino acid sequence of SEQ ID NO:30 (COV_S_T2_16).

According to the invention there is provided an isolated polypeptide, which comprises an amino acid sequence of SEQ ID NO:31 (COV_S_T2_17).

According to the invention there is provided an isolated polypeptide, which comprises an amino acid sequence of SEQ ID NO:32 (COV_S_T2_18).

According to the invention there is also provided an isolated polypeptide which comprises a coronavirus S protein RBD domain with at least one of the amino acid residues at positions corresponding to the amino acid residue positions of SEQ ID NO:11, as shown in Table 8.2 above.

Optionally an isolated polypeptide of the invention which comprises a coronavirus S protein RBD domain with at least one of the amino acid residues at positions corresponding to the amino acid residue positions of SEQ ID NO:11, as shown in Table 8.2 above, comprises at least five amino acid residues at positions corresponding to the amino acid residue positions of SEQ ID NO: 11, as shown in Table 8.2 above.

Optionally an isolated polypeptide of the invention which comprises a coronavirus S protein RBD domain with at least one of the amino acid residues at positions corresponding to the amino acid residue positions of SEQ ID NO:11, as shown in Table 8.2 above, comprises at least ten amino acid residues at positions corresponding to the amino acid residue positions of SEQ ID NO: 11, as shown in Table 8.2 above.

Optionally an isolated polypeptide of the invention which comprises a coronavirus S protein RBD domain with at least one of the amino acid residues at positions corresponding to the amino acid residue positions of SEQ ID NO:11, as shown in Table 8.2 above, comprises at least fifteen amino acid residues at positions corresponding to the amino acid residue positions of SEQ ID NO: 11, as shown in Table 8.2 above.

Optionally an isolated polypeptide of the invention which comprises a coronavirus S protein RBD domain with at least one of the amino acid residues at positions corresponding to the amino acid residue positions of SEQ ID NO: 11, as shown in Table 8.2 above, comprises all of the amino acid residues at positions corresponding to the amino acid residue positions of SEQ ID NO: 11, as shown in Table 8.2 above.

Optionally an isolated polypeptide of the invention which comprises a coronavirus S protein RBD domain with at least one, five, ten, fifteen, or all, of the amino acid residues at positions corresponding to the amino acid residue positions of SEQ ID NO: 11, as shown in Table 8.2 above further comprises at least one, or all of the amino acid residues at positions corresponding to the amino acid residue positions of SEQ ID NO:11, as shown in Table 8.3 above.

Optionally an isolated polypeptide of the invention which comprises a coronavirus S protein RBD domain with at least one, five, ten, fifteen, or all, of the amino acid residues at positions corresponding to the amino acid residue positions of SEQ ID NO:11, as shown in Table 8.2 above and at least one, or all of the amino acid residues at positions corresponding to the amino acid residue positions of SEQ ID NO:11, as shown in Table 8.3 above, further comprises at least one, or all of the amino acid residues at positions corresponding to the amino acid residue positions of SEQ ID NO: 11, as shown in any of Tables 8.4 to 8.6 above.

Optionally an isolated polypeptide of the invention which comprises a coronavirus S protein RBD domain comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:5.

Optionally an isolated polypeptide of the invention which comprises a coronavirus S protein RBD domain comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:11.

There is also provided according to the invention an isolated polypeptide comprising an amino acid sequence of SEQ ID NO:92 (COV_S_T2_17+tPA signal sequence).

### Discontinuous Epitope Sequences of Designed S Protein RBD Sequences COV_S_T2_14-18 (SEQ ID NOs: 28-32)

The sequence alignment below shows the designed S protein RBD sequences COV_S_T2_13-18 aligned. The figure shows the residues of discontinuous epitopes present in sequences COV_S_T2_14-18 shown in different format (bold and underline; bold and italics; and underline). The changes made relative to the COV_S_T2_13 sequence to provide discontinuous epitopes that elicit a broader or more potent immune response are shown by the vertical boxed regions:

| | | |
|---|---|---|
| >COV_S_T2_13 >COV_S_T2_14 >COV_S_T2_15 >COV_S_T2_16 >COV_S_T2_17 >COV_S_T2_18 | 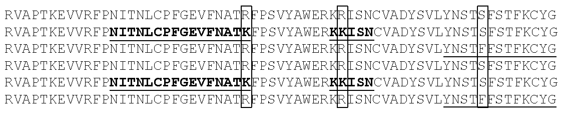 | 63 63 63 63 63 63 |
| >COV_S_T2_13 >COV_S_T2_14 >COV_S_T2_15 >COV_S_T2_16 >COV_S_T2_17 >COV_S_T2_18 | 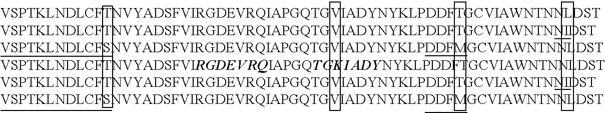 | 126 126 126 126 126 126 |
| >COV_S_T2_13 >COV_S_T2_14 >COV_S_T2_15 >COV_S_T2_16 >COV_S_T2_17 >COV_S_T2_18 | 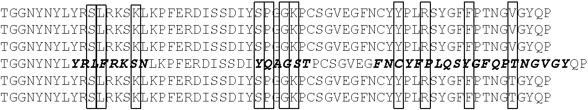 | 189 189 189 189 189 189 |
| >COV_S_T2_13 | YRVVVLSFELLNAPATVCGPKLSTD | 214 |
| >COV_S_T2_14 | YRVVVLSFELLNAPATVCGPKLSTD | 214 |
| >COV_S_T2_15 | YRVVVLSFELLNAPATVCGPKLSTD | 214 |
| >COV_S_T2_16 | YRVVVLSFELLNAPATVCGPKLSTD | 214 |
| >COV_S_T2_17 | YRVVVLSFELLNAPATVCGPKLSTD | 214 |
| >COV_S_T2_18 | YRVVVLSFELLNAPATVCGPKLSTD | 214 |

The residues of the discontinuous epitope present in COV_S_T2_14 and COV_S_T2_17 (marked in bold and underline) are as follows:
i) NITNLCPFGEVFNATK (SEQ ID NO:57)—residues 13-28;
ii) KKISN (SEQ ID NO:58)—residues 38-42;
iii) NI (SEQ ID NO:59)—residues 122-123

The residues of the discontinuous epitope present in COV_S_T2_15 and COV_S_T2_18 (marked in underline) are as follows:
i) YNSTFFSTFKCYGVSPTKLNDLCFS (SEQ ID NO:60)—residues 51-75;
ii) DDFM (SEQ ID NO:61)—residues 109-112
iii) FELLN (SEQ ID NO:62)—residues 197-201

The residues of the discontinuous epitope present in COV_S_T2_16 (marked in bold and italics) are as follows:
i) RGDEVRQ (SEQ ID NO:63)—residues 85-91;
ii) TGKIADY (SEQ ID NO:64)—residues 97-103;
iii) YRLFRKSN (SEQ ID NO:65)—residues 135-142;
iv) YQAGST (SEQ ID NO:66)—residues 155-160
v) FNCYFPLQSYGFQPTNGVGY (SEQ ID NO:67)—residues 168-187

The residues of the discontinuous epitope present in COV_S_T2_13, COV_S_T2_15, COV_S_T2_16, and COV_S_T2_18 (vertically adjacent the epitope marked in bold and underline) are as follows:
(i) NITNLCPFGEVFNATR (SEQ ID NO:68)—residues 13-28;
(ii) KRISN (SEQ ID NO:69)—residues 38-42;
(iii) NL (SEQ ID NO:70)—residues 122-123

The residues of the discontinuous epitope present in COV_S_T2_13, COV_S_T2_14, COV_S_T2_16, and COV_S_T2_17 (vertically adjacent the epitope marked in underline) are as follows:
(i) YNSTSFSTFKCYGVSPTKLNDLCFT (SEQ ID NO:71)—residues 51-75;
(ii) DDFT (SEQ ID NO:72)—residues 109-112
(iii) FELLN (SEQ ID NO:62)—residues 197-201

The residues of the discontinuous epitope present in COV_S_T2_13, COV_S_T2_14, and COV_S_T2_15 (vertically adjacent the epitope marked in bold and italics) are as follows:
(i) RGDEVRQ (SEQ ID NO:63)—residues 85-91;
(ii) TGVIADY (SEQ ID NO:73)—residues 97-103;
(iii) YRSLRKSK (SEQ ID NO:74)—residues 135-142;
(iv) YSPGGK (SEQ ID NO:75)—residues 155-160
(v) FNCYYPLRSYGFFPTNGVGY (SEQ ID NO:76)—residues 168-187

The residues of the discontinuous epitope present in COV_S_T2_17 and COV_S_T2_18 (vertically adjacent the epitope marked in bold and italics) are as follows:
(i) RGDEVRQ (SEQ ID NO:63)—residues 85-91;
(ii) TGVIADY (SEQ ID NO:73)—residues 97-103;
(iii) YRSLRKSK (SEQ ID NO:74)—residues 135-142;
(iv) YSPGGK (SEQ ID NO:75)—residues 155-160
(v) FNCYYPLRSYGFFPTNGTGY (SEQ ID NO:77)—residues 168-187

According to the invention there is provided an isolated polypeptide comprising an amino acid sequence with the following discontinuous amino acid sequences:

i)
(SEQ ID NO: 57)
NITNLCPFGEVFNATK;
ii)
(SEQ ID NO: 58)
KKISN;
iii)
(SEQ ID NO: 59)
NI.

According to the invention there is provided an isolated polypeptide comprising an amino acid sequence with the following discontinuous amino acid sequences:

i)
(SEQ ID NO: 60)
YNSTFFSTFKCYGVSPTKLN DLCFS;
ii)
(SEQ ID NO: 61)
DDFM;
iii)
(SEQ ID NO: 62)
FELLN.

According to the invention there is provided an isolated polypeptide comprising an amino acid sequence with the following discontinuous amino acid sequences:

i)
(SEQ ID NO: 63)
RGDEVRQ;
ii)
(SEQ ID NO: 64)
TGKIADY;
iii)
(SEQ ID NO: 65)
YRLFRKSN;
iv)
(SEQ ID NO: 66)
YQAGST;
v)
(SEQ ID NO: 67)
FNCYFPLQSYGFQPTNGVGY.

Optionally one or more residues of the amino acid residues of SEQ ID NOs: 63-67 in a polypeptide of the invention comprising discontinuous amino acid sequences of SEQ ID NOs: 63-67 may be changed (for example, by substitution or deletion) to provide a glycosylation site.

According to the invention there is also provided an isolated polypeptide comprising an amino acid sequence with the following discontinuous amino acid sequences:

(i)
(SEQ ID NO: 68)
NITNLCPFGEVFNATR;
ii)
(SEQ ID NO: 69)
KRISN;
(iii)
(SEQ ID NO: 70)
NL

According to the invention there is provided an isolated polypeptide comprising an amino acid sequence with the following discontinuous amino acid sequences:

(i)
(SEQ ID NO: 71)
YNSTSFSTFKCYGVSPTKLNDLCFT;
(ii)
(SEQ ID NO: 72)
DDFT
(iii)
(SEQ ID NO: 62)
FELLN

According to the invention there is provided an isolated polypeptide comprising an amino acid sequence with the following discontinuous amino acid sequences:

(i)
(SEQ ID NO: 63)
RGDEVRQ;
(ii)
(SEQ ID NO: 73)
TGVIADY;
(iii)
(SEQ ID NO: 74)
YRSLRKSK;
(iv)
(SEQ ID NO: 75)
YSPGGK
(v)
(SEQ ID NO: 76)
FNCYYPLRSYGFFPTNGVGY

According to the invention there is provided an isolated polypeptide comprising an amino acid sequence with the following discontinuous amino acid sequences:

(i)
(SEQ ID NO: 63)
RGDEVRQ;
(ii)
(SEQ ID NO: 73)
TGVIADY;
(iii)
(SEQ ID NO: 74)
YRSLRKSK;
(iv)
(SEQ ID NO: 75)
YSPGGK
(v)
(SEQ ID NO: 77)
FNCYYPLRSYGFFPTNGTGY

Optionally the discontinuous amino acid sequences of each polypeptide of the invention are present in the order recited.

Optionally each discontinuous amino acid sequence is separated by at least 3 amino acid residues from an adjacent discontinuous amino acid sequence.

Optionally each discontinuous amino acid sequence is separated by upto 100 amino acid residues from an adjacent discontinuous amino acid sequence.

Optionally a polypeptide of the invention comprising the recited discontinuous amino acid sequences is up to 250, 500, 750, 1,000, 1,250, or 1,500 amino acid residues in length.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28, comprises the following discontinuous amino acid sequences:

i)
(SEQ ID NO: 57)
NITNLCPFGEVFNATK;
ii)
(SEQ ID NO: 58)
KKISN;
iii)
(SEQ ID NO: 59)
NI.

Optionally the discontinuous amino acid sequences (i), (ii), and (iii) are at amino acid residue positions corresponding to (i) residues 13-28; (ii) residues 38-42; and (iii) residues 122-123 of SEQ ID NO:28, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29, comprises the following discontinuous amino acid sequences:

i)
(SEQ ID NO: 60)
YNSTFFSTFKCYGVSPTKLNDLCFS;
ii)
(SEQ ID NO: 61)
DDFM;
iii)
(SEQ ID NO: 62)
FELLN.

Optionally the discontinuous amino acid sequences (i), (ii), and (iii) are at amino acid residue positions corresponding to (i) residues 51-75; (ii) residues 109-112; and (iii) residues 197-201 of SEQ ID NO:29, respectively.

Optionally an isolated polypeptide of the invention comprising an amino acid sequence which has at least 93%, 94%, 95%, 96%, 97%, 98%, or 99% amino acid identity over its entire length with the amino acid sequence of SEQ ID NO:30, comprises the following discontinuous amino acid sequences:

i)
(SEQ ID NO: 63)
RGDEVRQ;
ii)
(SEQ ID NO: 64)
TGKIADY;
iii)
(SEQ ID NO: 65)
YRLFRKSN;
iv)
(SEQ ID NO: 66)
YQAGST;
v)
(SEQ ID NO: 67)
FNCYFPLQSYGFQPTNGVGY.

Optionally the discontinuous amino acid sequences (i), (ii), (iii), (iv), and (v) are at amino acid residue positions corresponding to (i) residues 85-91, (ii) residues 97-103, (iii) residues 135-142, (iv) residues 155-160, and (v) residues 168-187 of SEQ ID NO:30, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31,

i)
(SEQ ID NO: 57)
NITNLCPFGEVFNATK;
ii)
(SEQ ID NO: 58)
KKISN;
iii)
(SEQ ID NO: 59)
NI.

Optionally the discontinuous amino acid sequences (i), (ii), and (iii) are at amino acid residue positions corresponding to (i) residues 13-28; (ii) residues 38-42; and (iii) residues 122-123 of SEQ ID NO:31, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32, comprises the following discontinuous amino acid sequences:

i)
(SEQ ID NO: 60)
YNSTFFSTFKCYGVSPTKLNDLCFS;
ii)
(SEQ ID NO: 61)
DDFM;
iii)
(SEQ ID NO: 62)
FELLN.

Optionally the discontinuous amino acid sequences (i), (ii), and (iii) are at amino acid residue positions corresponding to (i) residues 51-75; (ii) residues 109-112; and (iii) residues 197-201 of SEQ ID NO:32, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29, comprises the following discontinuous amino acid sequences:

(i)
(SEQ ID NO: 68)
NITNLCPFGEVFNATR;
(ii)
(SEQ ID NO: 69)
KRISN;
(iii)
(SEQ ID NO: 70)
NL

Optionally the discontinuous amino acid sequences (i), (ii), and (iii) are at amino acid residue positions corresponding to (i) residues 13-28; (ii) residues 38-42; and (iii) residues 122-123 of SEQ ID NO:29, respectively.

Optionally an isolated polypeptide of the invention comprising an amino acid sequence which has at least 93%, 94%, 95%, 96%, 97%, 98%, or 99% amino acid identity over its entire length with the amino acid sequence of SEQ ID NO:30, comprises the following discontinuous amino acid sequences:

(i)
(SEQ ID NO: 68)
NITNLCPFGEVFNATR;
ii)
(SEQ ID NO:69)
KRISN;
(iii)
(SEQ ID NO: 70)
NL

Optionally the discontinuous amino acid sequences (i), (ii), and (iii) are at amino acid residue positions corresponding to (i) residues 13-28; (ii) residues 38-42; and (iii) residues 122-123 of SEQ ID NO:30, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32, comprises the following discontinuous amino acid sequences:

(i)
(SEQ ID NO: 68)
NITNLCPFGEVFNATR;
(ii)
(SEQ ID NO: 69)
KRISN;
(iii)
(SEQ ID NO: 70)
NL

Optionally the discontinuous amino acid sequences (i), (ii), and (iii) are at amino acid residue positions corresponding to (i) residues 13-28; (ii) residues 38-42; and (iii) residues 122-123 of SEQ ID NO:32, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28, comprises the following discontinuous amino acid sequences:

(i)
(SEQ ID NO: 71)
YNSTSFSTFKCYGVSPTKLNDLCFT;
(ii)
(SEQ ID NO: 72)
DDFT
(iii)
(SEQ ID NO: 62)
FELLN

Optionally the discontinuous amino acid sequences (i), (ii), and (iii) are at amino acid residue positions corresponding to (i) residues 51-75; (ii) residues 109-112; and (iii) residues 197-201 of SEQ ID NO:28, respectively.

Optionally an isolated polypeptide of the invention comprising an amino acid sequence which has at least 93%, 94%, 95%, 96%, 97%, 98%, or 99% amino acid identity over its entire length with the amino acid sequence of SEQ ID NO:30, comprises the following discontinuous amino acid sequences:

(i)
(SEQ ID NO: 71)
YNSTSFSTFKCYGVSPTKLNDLCFT;
(ii)
(SEQ ID NO: 72)
DDFT
(iii)
(SEQ ID NO: 62)
FELLN

Optionally the discontinuous amino acid sequences (i), (ii), and (iii) are at amino acid residue positions corresponding to (i) residues 51-75; (ii) residues 109-112; and (iii) residues 197-201 of SEQ ID NO:30, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31, comprises the following discontinuous amino acid sequences:

(i)
(SEQ ID NO: 71)
YNSTSFSTFKCYGVSPTKLNDLCFT;
(ii)
(SEQ ID NO: 72)
DDFT
(iii)
(SEQ ID NO: 62)
FELLN

Optionally the discontinuous amino acid sequences (i), (ii), and (iii) are at amino acid residue positions corresponding to (i) residues 51-75; (ii) residues 109-112; and (iii) residues 197-201 of SEQ ID NO:31, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28, comprises the following discontinuous amino acid sequences:

(i)
(SEQ ID NO: 63)
RGDEVRQ;
(ii)
(SEQ ID NO: 73)
TGVIADY;
(iii)
(SEQ ID NO: 74)
YRSLRKSK;
(iv)
(SEQ ID NO: 75)
YSPGGK
(v)
(SEQ ID NO: 76)
FNCYYPLRSYGFFPTNGVGY

Optionally the discontinuous amino acid sequences (i), (ii), and (iii) are at amino acid residue positions corresponding to (i) residues 51-75; (ii) residues 109-112; and (iii) residues 197-201 of SEQ ID NO:28, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29, comprises the following discontinuous amino acid sequences:

(i)
(SEQ ID NO: 63)
RGDEVRQ;
(ii)
(SEQ ID NO: 73)
TGVIADY;
(iii)
(SEQ ID NO: 74)
YRSLRKSK;
(iv)
(SEQ ID NO: 75)
YSPGGK
(v)
(SEQ ID NO: 76)
FNCYYPLRSYGFFPTNGVGY

Optionally the discontinuous amino acid sequences (i), (ii), and (iii) are at amino acid residue positions corresponding to (i) residues 51-75; (ii) residues 109-112; and (iii) residues 197-201 of SEQ ID NO:29, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31, comprises the following discontinuous amino acid sequences:

(i)
(SEQ ID NO: 63)
RGDEVRQ;
(ii)
(SEQ ID NO: 73)
TGVIADY;
(iii)
(SEQ ID NO: 74)
YRSLRKSK;
(iv)
(SEQ ID NO: 75)
YSPGGK
(v)
(SEQ ID NO: 77)
FNCYYPLRSYGFFPTNGTGY

Optionally the discontinuous amino acid sequences (i), (ii), and (iii) are at amino acid residue positions corresponding to (i) residues 51-75; (ii) residues 109-112; and (iii) residues 197-201 of SEQ ID NO:31, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32, comprises the following discontinuous amino acid sequences:

(i)
(SEQ ID NO: 63)
RGDEVRQ;
(ii)
(SEQ ID NO: 73)
TGVIADY;
(iii)
(SEQ ID NO: 74)
YRSLRKSK;
(iv)
(SEQ ID NO: 75)
YSPGGK
(v)
(SEQ ID NO: 77)
FNCYYPLRSYGFFPTNGTGY

Optionally the discontinuous amino acid sequences (i), (ii), and (iii) are at amino acid residue positions corresponding to (i) residues 51-75; (ii) residues 109-112; and (iii) residues 197-201 of SEQ ID NO:32, respectively.

Designed Coronavirus S Protein RBD Sequences with Altered Glycosylation Sites

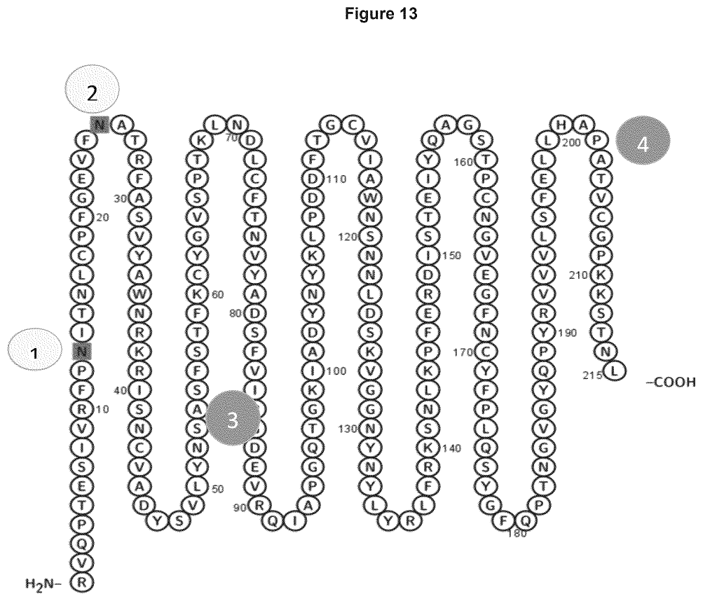

Masking/de-masking of epitopes has been shown to alter the immune response by masking non-neutralising epitopes, or by de-masking important epitopes in MERS (Du L et. al., Nat. Comm, volume 7, Article number: 13473 (2016)). We have prepared additional designed S protein RBD sequences (SARS2 RBD designs M7, M8, M9, and M10) in which we have deleted a glycosylation site of SARS2 RBD sequence, or introduced a glycosylation site to SARS2 RBD sequence. The changes made are illustrated in FIG. 13, and discussed in Example 14 below. Designs M7 and M9 include a glycosylation site introduced at the position indicated by circled number 4 (residue position 203) in FIG. 13. Designs M8 and M10 include a deleted glycosylation site at each of the positions indicated by circled numbers 1 and 2 (residue positions 13 and 25, respectively) in FIG. 13. The M8 design also includes an introduced glycosylation site at the position indicated by circled number 3 (residue position 54).

The amino acid sequences of SARS2 RBD designs M7, M8, M9, and M10 are shown below, and in Example 14:

>M7
(SEQ ID NO: 33)
RVQPTESIVR FPNITNLCPF GEVFNATRFA SVYAWNRKRI SNCVADYSVL YNSASFSTFK CYGVSPTKLN
DLCFTNVYAD SFVIRGDEVR QIAPGQTGKI ADYNYKLPDD FTGCVIAWNS NNLDSKVGGN YNYLYRLFRK
SNLKPFERDI STEIYQAGST PCNGVEGFNC YFPLQSYGFQ PTNGVGYQPY RVVVLSFELL HANATVCGPK
KSTN
>M8
(SEQ ID NO: 34)
RVQPTESIVR FPQITNLCPF GEVFQATRFA SVYAWNRKRI SNCVADYSVL YNSTSFSTFK CYGVSPTKLN
DLCFTNVYAD SFVIRGDEVR QIAPGQTGKI ADYNYKLPDD FTGCVIAWNS NNLDSKVGGN YNYLYRLFRK
SNLKPFERDI STEIYQAGST PCNGVEGFNC YFPLQSYGFQ PTNGVGYQPY RVVVLSFELL HAPATVCGPK
KSTN
>M9
(SEQ ID NO: 35)
RVSPTQEVVR FPNITNLCPF DKVFNATRFP SVYAWERTKI SDCVADYTVL YNSTSFSTFK CYGVSPSKLI
DLCFTSVYAD TFLIRCSEVR QVAPGQTGVI ADYNYKLPDD FTGCVIAWNT AKQDTGSSGN YNYYYRSHRK
TKLKPFERDL SSDECSPDGK PCTPPAFNGV RGFNCYFTLS TYDFNPNVPV EYQATRVVVL SFELLNANAT
VCGPKLSTQ
>M10
(SEQ ID NO: 36)
RVSPTQEVVR FPQITNLCPF DKVFQATRFP SVYAWERTKI SDCVADYTVL YNSTSFSTFK CYGVSPSKLI
DLCFTSVYAD TFLIRCSEVR QVAPGQTGVI ADYNYKLPDD FTGCVIAWNT AKQDTGSSGN YNYYYRSHRK
TKLKPFERDL SSDECSPDGK PCTPPAFNGV RGFNCYFTLS TYDFNPNVPV EYQATRVVVL SFELLNAPAT
VCGPKLSTQ

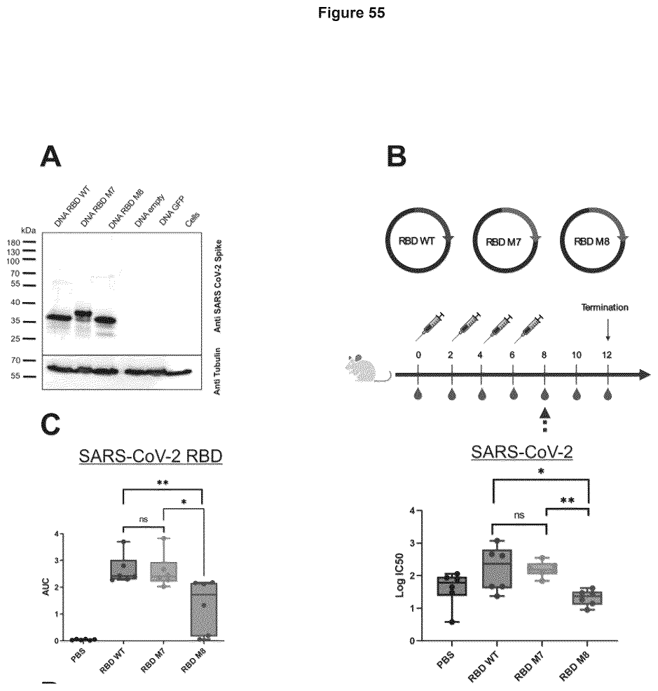

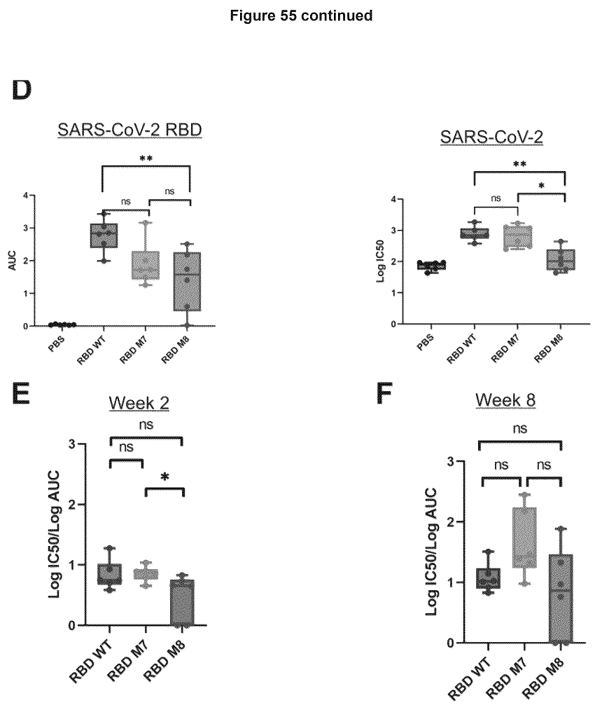

Such polypeptides are particularly advantageous as they elicit broadly neutralising antibody responses to a diverse panel of coronavirus VOCs, as demonstrated by the results described FIGS. 55-59, and Example 38. In particular, heterologous immunisation using M7 DNA prime followed by M7 MVA boost results in significantly higher titres of neutralising antibodies against panel of VOCs (Wuhan-1 B, Alpha B.1.1.7, Beta B.1.351, Gamma P.1, Delta B.1.617.2, and Omicron BA.1) compared with homologous immunisation of M7 DNA prime followed by M7 DNA boost (FIG. 57C). The strongest nAb response could be observed in MVA RBD M7 boosted mice against Wuhan-1 B, Alpha B.1.1.7, Gamma P.1, Delta B.1.617.2 variants. Furthermore, M7 DNA prime followed by M7 MVA boost elicited significantly higher titres of neutralising antibodies against Wuhan-1 B, Alpha B.1.1.7, Gamma P.1, Delta B.1.617.2 compared to heterologous DNA prime/MVA boost with WT RBD, and comparable neutralisation against Beta B.1.351 and Omicron BA.1 and BA.2.

Alignment of these sequences with the SARS2 Reference sequence (EPI_ISL_402119_RBD (CoV_T2_6) (SEQ ID NO:11)) is shown in Example 14 below.

The amino acid differences of the designed sequences from the SARS2 reference sequence are shown in Table 9 below (with differences from the reference sequence highlighted in bold):

TABLE 9

| | SARS2 RBD | | | | | |
|---|---|---|---|---|---|---|
| Circled | (SEQ ID | | M7 residue | M8 residue | M9 residue | M10 residue |
| number of | NO: 11) residue | Reference | (SEQ ID | (SEQ ID | (SEQ ID | (SEQ ID |
| FIG. 13 | position | residue | NO: 33) | NO: 34) | NO: 35) | NO: 36) |
| | 3 | Q | | | S | S |
| | 6 | E | | | Q | Q |
| | 7 | S | | | E | E |
| | 8 | I | | | V | V |
| 1 | 13 | N | | Q | | Q |
| | 21 | G | | | D | K |
| | 22 | E | | | D | K |
| 2 | 25 | N | | Q | | Q |
| | 30 | A | | | P | P |
| | 36 | N | | | E | E |
| | 38 | K | | | T | K |
| | 39 | R | | | T | K |
| | 42 | N | | | D | D |
| | 48 | S | | | T | T |
| 3 | 54 | A | | T | T | T |
| | 67 | T | | | S | S |
| | 70 | N | | | I | I |
| | 76 | N | | | S | S |
| | 81 | S | | | T | T |
| | 83 | V | | | L | L |
| | 86 | G | | | C | C |
| | 87 | D | | | S | S |
| | 92 | I | | | V | V |
| | 99 | K | | | V | V |
| | 120 | S | | | T | T |
| | 121 | N | | | A | A |
| | 122 | N | | | K | K |
| | 123 | L | | | Q | Q |
| | 125 | S | | | T | T |
| | 126 | K | | | G | G |
| | 127 | V | | | S | S |
| | 128 | G | | | S | S |
| | 134 | L | | | Y | Y |
| | 137 | L | | | S | S |
| | 138 | F | | | H | H |
| | 141 | S | | | T | T |
| | 142 | N | | | K | K |
| | 150 | I | | | L | L |
| | 152 | T | | | S | S |
| | 153 | E | | | D | D |
| | 154 | I | | | E | E |
| | 155 | Y | | | C | C |
| | 156 | Q | | | S | S |
| | 157 | A | | | P | P |
| | 158 | G | | | D | D |
| | 159 | S | | | G | G |
| | 160 | T | | | K | K |
| | * | — | | | T | T |
| | * | — | | | P | P |
| | * | — | | | P | P |
| | * | — | | | A | A |
| | * | — | | | F | F |
| | 166 | E | | | R | R |
| | 173 | P | | | T | T |
| | 175 | Q | | | S | S |
| | 176 | S | | | T | T |
| | 178 | G | | | D | D |
| | 180 | Q | | | N | N |
| | 182 | T | | | N | N |
| | 183 | N | | | V | V |
| | 184 | G | | | P | P |
| | 186 | E | | | E | E |
| | 189 | P | | | A | A |
| | 190 | Y | | | T | T |
| | 201 | H | | | N | N |
| 4 | 203 | P | N | | N | — |
| | 211 | K | | | L | L |
| | 214 | Q | | | Q | Q |
| Total no of | | | 1 | 3 | 66 | 67 |
| differences | | | | | | |
| from | | | | | | |
| reference | | | | | | |
| Percentage | | | 99.53% | 98.60% | 69.12% | 68.69% |
| identity with | | | | | | |
| reference | | | | | | |

* Residues inserted between amino acid residue positions 162 and 163 of SEQ ID NO: 11.

According to the invention there is provided an isolated polypeptide, which comprises an amino acid sequence according to SEQ ID NO:33 (Designed S protein RBD sequence M7).

According to the invention there is provided an isolated polypeptide, which comprises an amino acid sequence according to SEQ ID NO:34 (Designed S protein RBD sequence M8).

According to the invention there is provided an isolated polypeptide, which comprises an amino acid sequence according to SEQ ID NO:35 (Designed S protein RBD sequence M9).

According to the invention there is provided an isolated polypeptide, which comprises an amino acid sequence according to SEQ ID NO:36 (Designed S protein RBD sequence M10).

According to the invention there is also provided an isolated polypeptide comprising an amino acid sequence of SEQ ID NO: 34 (M8), or an amino acid sequence which has at least 99% amino acid identity over its entire length with the amino acid sequence of SEQ ID NO:34.

Optionally a polypeptide of the invention comprising an amino acid sequence of SEQ ID NO:34 (M8), or an amino acid sequence which has at least 99% amino acid identity over its entire length with the amino acid sequence of SEQ ID NO:34, comprises at least one, or all of the following amino acid residues at positions corresponding to the amino acid residue positions of SEQ ID NO: 11: 13Q, 25Q, 54T.

According to the invention there is also provided an isolated polypeptide which comprises a coronavirus S protein RBD domain with at least one of the following amino acid residues at positions corresponding to the amino acid residue positions of SEQ ID NO:11: 13Q, 25Q, 54T, 203N.

According to the invention there is also provided an isolated polypeptide comprising an amino acid sequence of SEQ ID NO: 35 (M9), or an amino acid sequence which has at least 70% amino acid identity over its entire length with the amino acid sequence of SEQ ID NO:35.

Optionally an isolated polypeptide of the invention which comprises an amino acid sequence of SEQ ID NO: 35 (M9), or an amino acid sequence which has at least 70% amino acid identity over its entire length with the amino acid sequence of SEQ ID NO:35, comprises at least one, or all of the amino acid residues at positions corresponding to the amino acid residue positions as shown in Table 9.1 below.

TABLE 9.1

| SARS2 RBD (SEQ ID NO: 11) | M9 residue |
|---|---|
| residue position | (SEQ ID NO: 35) |
| 3 | S |
| 6 | Q |
| 7 | E |
| 8 | V |
| 21 | D |
| 22 | D |
| 30 | P |
| 36 | E |
| 38 | T |
| 39 | T |
| 42 | D |
| 48 | T |
| 54 | T |
| 67 | S |
| 70 | I |
| 76 | S |
| 81 | T |
| 83 | L |
| 86 | C |
| 87 | S |
| 92 | V |
| 99 | V |
| 120 | T |
| 121 | A |
| 122 | K |
| 123 | Q |
| 125 | T |
| 126 | G |
| 127 | S |
| 128 | S |
| 134 | Y |
| 137 | S |
| 138 | H |
| 141 | T |
| 142 | K |
| 150 | L |
| 152 | S |
| 153 | D |
| 154 | E |
| 155 | C |
| 156 | S |
| 157 | P |
| 158 | D |
| 159 | G |
| 160 | K |
| * | T |
| * | P |
| * | P |
| * | A |
| * | F |
| 166 | R |
| 173 | T |
| 175 | S |
| 176 | T |
| 178 | D |
| 180 | N |
| 182 | N |
| 183 | V |
| 184 | P |
| 186 | E |
| 189 | A |
| 190 | T |
| 201 | N |
| 203 | N |
| 211 | L |
| 214 | Q |

* Residues for insertion between amino acid residue positions 162 and 163 of SEQ ID NO: 11.

Optionally an isolated polypeptide of the invention which comprises an amino acid sequence of SEQ ID NO: 35 (M9), or an amino acid sequence which has at least 70% amino acid identity over its entire length with the amino acid sequence of SEQ ID NO:35, comprises at least one, or both of the following amino acid residues at positions corresponding to the amino acid residue positions of SEQ ID NO: 11: 54T, 203N.

According to the invention there is also provided an isolated polypeptide comprising an amino acid sequence of SEQ ID NO: 36 (M10), or an amino acid sequence which has at least 69% amino acid identity over its entire length with the amino acid sequence of SEQ ID NO:36.

Optionally an isolated polypeptide of the invention which comprises an amino acid sequence of SEQ ID NO: 36 (M10), or an amino acid sequence which has at least 69% amino acid identity over its entire length with the amino acid sequence of SEQ ID NO:36, comprises at least one, or all of the amino acid residues at positions corresponding to the amino acid residue positions as shown in Table 9.2 below.

TABLE 9.2

| SARS2 RBD (SEQ ID NO: 11) | M10 residue |
|---|---|
| residue position | (SEQ ID NO: 36) |
| 3 | S |
| 6 | Q |
| 7 | E |
| 8 | V |
| 13 | Q |
| 21 | K |
| 22 | K |
| 25 | Q |
| 30 | P |
| 36 | E |
| 38 | K |
| 39 | K |
| 42 | D |
| 48 | T |
| 54 | T |
| 67 | S |
| 70 | I |
| 76 | S |
| 81 | T |
| 83 | L |
| 86 | C |
| 87 | S |
| 92 | V |
| 99 | V |
| 120 | T |
| 121 | A |
| 122 | K |
| 123 | Q |
| 125 | T |
| 126 | G |
| 127 | S |
| 128 | S |
| 134 | Y |
| 137 | S |
| 138 | H |
| 141 | T |
| 142 | K |
| 150 | L |
| 152 | S |
| 153 | D |
| 154 | E |
| 155 | C |
| 156 | S |
| 157 | P |
| 158 | D |
| 159 | G |
| 160 | K |
| * | T |
| * | P |
| * | P |
| * | A |
| * | F |
| 166 | R |
| 173 | T |
| 175 | S |
| 176 | T |
| 178 | D |
| 180 | N |
| 182 | N |
| 183 | V |
| 184 | P |
| 186 | E |
| 189 | A |
| 190 | T |
| 201 | N |
| 211 | L |
| 214 | Q |

* Residues for insertion between amino acid residue positions 162 and 163 of SEQ ID NO: 11.

Optionally an isolated polypeptide of the invention which comprises an amino acid sequence of SEQ ID NO: 36 (M10), or an amino acid sequence which has at least 69% amino acid identity over its entire length with the amino acid sequence of SEQ ID NO:36, comprises at least one, or all of the following amino acid residues at positions corresponding to the amino acid residue positions of SEQ ID NO: 11: 13Q, 25Q, 54T.

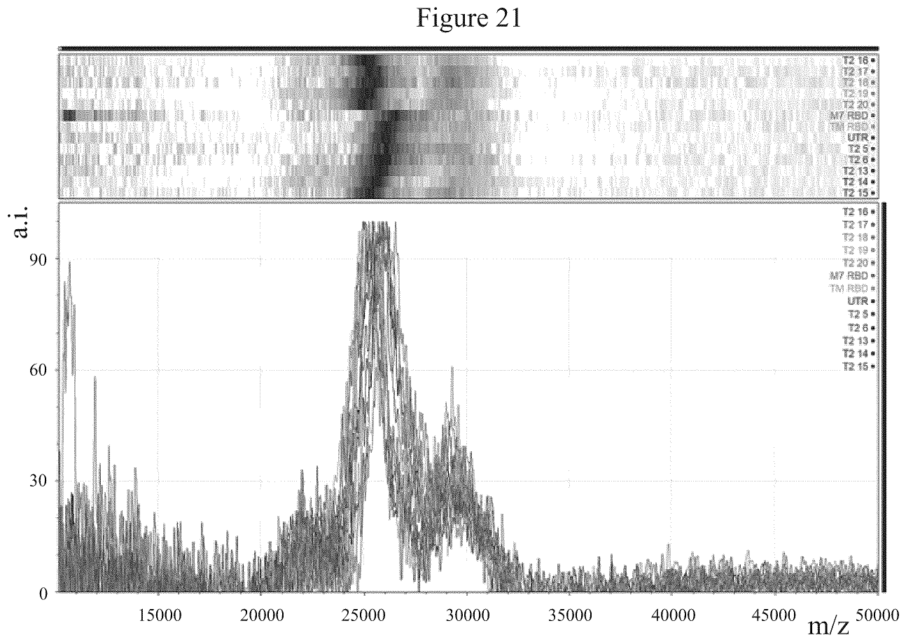

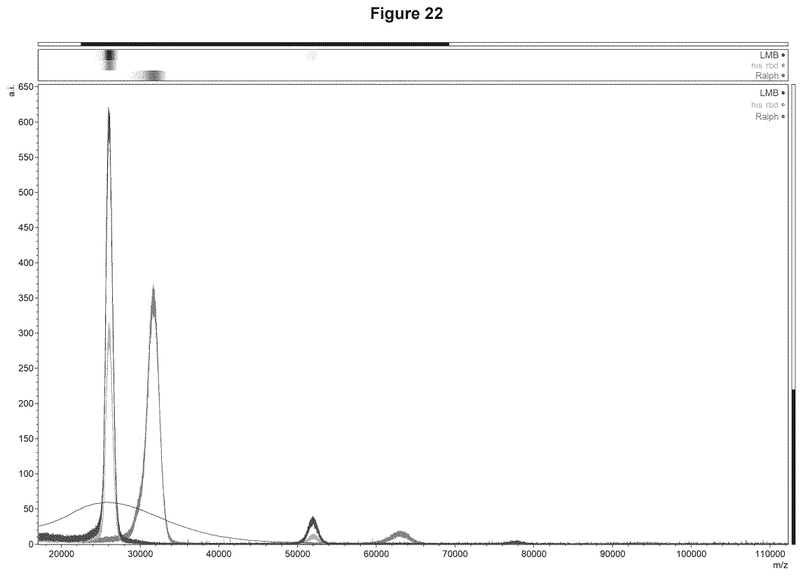

The effect of glycosylation of the RBD protein is believed to be important. We have found that M7 and wild-type SARS2 RBD DNA (believed to result in expression of glycosylated RBD protein) is superior to recombinant SARS2 RBD protein (non-glycosylated, or sparsely glycosylated) in inducing neutralising responses to SARS2. Example 28 below describes Mass spectroscopy data obtained to study glycosylation of SARS-CoV-2 (SARS2) RBD proteins in supernatants derived from HEK cells transfected with pEVAC plasmid encoding SARS-CoV-2 RBD sequences, compared with recombinant SARS-CoV-2 RBD proteins (see FIGS. 21 and 22). It was concluded from the results that there are two main glycosylated forms of the proteins obtained from the supernatants, in comparison to purified (recombinant) protein. The purified protein is non-glycosylated or sparsely glycosylated. This difference in glycosylation is believed to be important, as the glycosylation sites surround the epitope region and are conserved in most sarbecoviruses. These glycosylation sites are also important for interaction with some of the antibodies.

Optionally a polypeptide of the invention comprising an amino acid sequence of a designed coronavirus spike(S) protein (full-length, truncated, or RBD) comprises at least one glycosylation site in the RBD sequence.

Optionally a polypeptide of the invention comprising an amino acid sequence of a designed coronavirus spike(S) protein (full-length, truncated, or RBD) comprises at least two glycosylation sites in the RBD sequence.

Optionally a polypeptide of the invention comprising an amino acid sequence of a designed coronavirus spike(S) protein (full-length, truncated, or RBD) comprises at least three glycosylation sites in the RBD sequence.

Optionally a polypeptide of the invention comprising an amino acid sequence of a designed coronavirus spike(S) protein (full-length, truncated, or RBD) comprises a glycosylation site located within the last 10 amino acids of the RBD sequence, preferably at a residue position corresponding to residue position 203 of the RBD sequence.

According to the invention there is also provided an isolated polypeptide, which comprises an amino acid sequence of a SARS2 RBD with a glycosylation site located within the last 10 amino acids of the SARS2 RBD sequence, preferably at a residue position corresponding to residue position 203 of the RBD sequence.

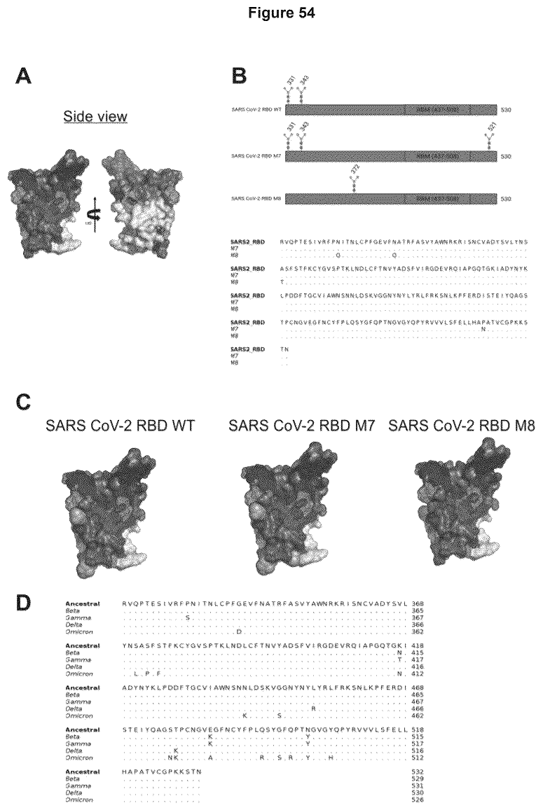

According to the invention there is also provided an isolated polypeptide, which comprises an amino acid sequence of a SARS2 RBD with a glycosylation site located within the epitope region of monoclonal antibody CR3022 (the epitope region of mAb CR3022 is shown in FIG. 54B).

We have also found that immunisation of mice with a wild-type SARS1 S protein, or RBD protein, or a wild-type SARS2 S protein, or RBD protein, induced antibodies that bind SARS2 RBD.

There is also provided according to the invention an isolated polypeptide comprising an amino acid sequence of SEQ ID NO:5.

There is also provided according to the invention an isolated polypeptide comprising an amino acid sequence of SEQ ID NO: 11.

A conventional way to produce cross-reactive antigens is to generate a consensus sequence based on natural diversity. Antigenic sequences encoded by nucleic acid sequences of the invention described herein account for sampling bias and coevolution between sites. The result is a realistic molecule which induces an immune response to a range of viruses. As a further refinement, we enrich the antigenic sequences for known and predicted epitopes. We have developed an algorithm to select the combination of epitopes that maximise population protection against a range of target viruses. This algorithm identifies conserved epitopes whilst penalising redundancy and ensuring that the selected epitopes are bound by a range of common MHC alleles.

To avoid disease enhancement we modify the antigens, deleting regions associated with immunopathology, often referred to as antibody dependent enhancement (ADE) and/or complement triggered, or virus triggered proinflammatory responses. In order to validate these modifications, we have developed assays to screen against such ADE-like effects. Using assays modified from Yip et al. (Yip et al. “Antibody-dependent infection of human macrophages by severe acute respiratory syndrome coronavirus”, Virol J. 2014; 11:82; Jaume et al. “Anti-Severe Acute Respiratory Syndrome Coronavirus Spike Antibodies Trigger Infection of Human Immune Cells via a pH- and Cysteine Protease-Independent FcγR Pathway” Journal Of Virology, October 2011, p. 10582-10597), non-neutralising antibodies to the non-RBD site of the S protein that allow SARS-CoV-1 to enter non-ACE2 expressing immune cells, which bear Fc-γ-RII, can be identified.

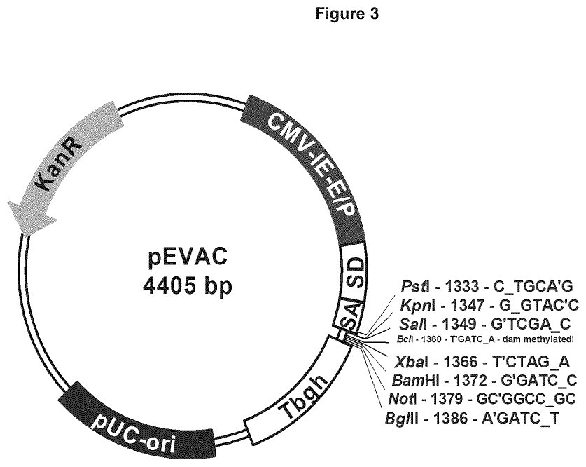

After designing antigens, DNA sequences encoding them are optimised for expression in mammalian cells. In this DNA form, multiple synthetic genes of the target antigens are inserted into a DNA plasmid vector (for example, pEVAC-see FIG. 3), which is used for both in vitro and in vivo immune screening.

### Designed Coronavirus Full-Length S Protein Sequence to Protect Against COVID-19 Variants

Multiple SARS-CoV-2 variants are circulating globally. Several new variants emerged in the fall of 2020, most notably:

In the United Kingdom (UK), a new variant of SARS-CoV-2 (known as 201/501Y.V1, VOC 202012/01, or B.1.1.7) emerged with a large number of mutations. This variant has since been detected in numerous countries around the world, including the United States (US). In January 2021, scientists from UK reported evidence that suggests the B.1.1.7 variant may be associated with an increased risk of death compared with other variants, although more studies are needed to confirm this finding. This variant was reported in the US at the end of December 2020.

In South Africa, another variant of SARS-CoV-2 (known as 20H/501Y.V2 or B. 1.351) emerged independently of B.1.1.7. This variant shares some mutations with B.1.1.7. Cases attributed to this variant have been detected in multiple countries outside of South Africa. This variant was reported in the US at the end of January 2021.

In Brazil, a variant of SARS-CoV-2 (known as P.1) emerged that was first was identified in four travelers from Brazil, who were tested during routine screening at Haneda airport outside Tokyo, Japan. This variant has 17 unique mutations, including three in the receptor binding domain of the spike protein. This variant was detected in the US at the end of January 2021. Scientists are working to learn more about these variants to better understand how easily they might be transmitted and the effectiveness of currently authorized vaccines against them. New information about the virologic, epidemiologic, and clinical characteristics of these variants is rapidly emerging.

As described in more detail in Example 30 below, we have designed a new full-length S protein sequence (referred to as “VOC Chimera”, or COV_S_T2_29) for use as a COVID-19 vaccine insert to protect against variants B.1.1.7, P.1, and B.1.351. The amino acid sequence of the designed full-length S protein sequence is given below, and in Example 30:

| >COV_S_T2_29 (VOC chimera) | |
|---|---|
| (SEQ ID NO: 53) | |
| MFVFLVLLPL VSSQCVNFTN RTQLPSAYTN SFTRGVYYPD KVFRSSVLHS TQDLFLPFFS | 60 |
| NVTWFHAISG TNGTKRFDNP VLPFNDGVYF ASTEKSNIIR GWIFGTTLDS KTQSLLIVNN | 120 |
| ATNVVIKVCE FQFCNDPFLG VYHKNNKSWM ESEFRVYSSA NNCTFEYVSQ PFLMDLEGKQ | 180 |
| GNFKNLREFV FKNIDGYFKI YSKHTPINLV RDLPQGFSAL EPLVDLPIGI NITRFQTLLA | 240 |
| LHRSYLTPGD SSSGWTAGAA AYYVGYLQPR TFLLKYNENG TITDAVDCAL DPLSETKCTL | 300 |
| KSFTVEKGIY QTSNFRVQPT ESIVRFPNIT NLCPFGEVFN ATRFASVYAW NRKRISNCVA | 360 |
| DYSVLYNSAS FSTFKCYGVS PTKLNDLCFT NVYADSFVIR GDEVRQIAPG QTGNIADYNY | 420 |
| KLPDDFTGCV IAWNSNNLDS KVGGNYNYLY RLFRKSNLKP FERDISTEIY QAGSTPCNGV | 480 |
| KGFNCYFPLQ SYGFQPTYGV GYQPYRVVVL SFELLHAPAT VCGPKKSTNL VKNKCVNFNF | 540 |
| NGLTGTGVLT ESNKKFLPFQ QFGRDIADTT DAVRDPQTLE ILDITPCSFG GVSVITPGTN | 600 |
| TSNQVAVLYQ GVNCTEVPVA IHADQLTPTW RVYSTGSNVF QTRAGCLIGA EHVNNSYECD | 660 |
| IPIGAGICAS YQTQTNSHRR ARSVASQSII AYTMSLGAEN SVAYSNNSIA IPTNFTISVT | 720 |
| TEILPVSMTK TSVDCTMYIC GDSTECSNLL LQYGSFCTQL NRALTGIAVE QDKNTQEVFA | 780 |
| QVKQIYKTPP IKDFGGFNFS QILPDPSKPS KRSFIEDLLF NKVTLADAGF IKQYGDCLGD | 840 |
| IAARDLICAQ KFNGLTVLPP LLTDEMIAQY TSALLAGTIT SGWTFGAGAA LQIPFAMQMA | 900 |
| YRFNGIGVTQ NVLYENQKLI ANQFNSAIGK IQDSLSSTAS ALGKLQDVVN QNAQALNTLV | 960 |
| KQLSSNFGAI SSVLNDILSR LDPPEAEVQI DRLITGRLQS LQTYVTQQLI RAAEIRASAN | 1020 |
| LAATKMSECV LGQSKRVDFC GKGYHLMSFP QSAPHGVVFL HVTYVPAQEK NFTTAPAICH | 1080 |
| DGKAHFPREG VFVSNGTHWF VTQRNFYEPQ IITTDNTFVS GNCDVVIGIV NNTVYDPLQP | 1140 |
| ELDSFKEELD KYFKNHTSPD VDLGDISGIN ASVVNIQKEI DRLNEVAKNL NESLIDLQEL | 1200 |
| GKYEQYIKWP WYIWLGFIAG LIAIVMVTIM LCCMTSCCSC LKGCCSCGSC CKFDEDDSEP | 1260 |
| VLKGVKLHYT | 1270 |

Alignment of this sequence with SARS2 Reference sequence (EPI_ISL_402130 (Wuhan strain) (SEQ ID NO:52)) is shown in Example 30 below.

The amino acid differences of the designed sequence COV_S_T2_29 (SEQ ID NO:53) from the SARS2 reference sequence (SEQ ID NO:52) are shown in Table 9.3 below:

TABLE 9.3

| SARS2 S protein | SARS2 Reference | COV_S_T2_29 |
|---|---|---|
| residue position | amino acid residue | amino acid residue |
| (SEQ ID NO: 52) | (SEQ ID NO: 52) | (SEQ ID NO: 53) |
| 18 | L | F |
| 20 | T | N |
| 26 | P | S |
| 69 | H | — (deletion) |
| 70 | V | — (deletion) |
| 144 | Y | — (deletion) |
| 417 | K | N |
| 484 | E | K |
| 501 | N | Y |
| 614 | D | G |
| 681 | P | H |
| 986 | K | P |
| 987 | V | P |

According to the invention there is provided an isolated polypeptide, which comprises an amino acid sequence of SEQ ID NO:53.

According to the invention there is also provided an isolated polypeptide comprising an amino acid sequence of SEQ ID NO:53, or an amino acid sequence which has at least 99% amino acid identity over its entire length with the amino acid sequence of SEQ ID NO:53.

Optionally a polypeptide of the invention comprising an amino acid sequence of SEQ ID NO: 53, or an amino acid sequence which has at least 99% amino acid identity over its entire length with the amino acid sequence of SEQ ID NO:53, comprises at least one, or all of the amino acid residues or deletions, at positions corresponding to the amino acid residue positions of SEQ ID NO:52, as shown in Table 9.4 below:

TABLE 9.4

| SARS2 S protein | COV_S_T2_29 |
|---|---|
| residue position | amino acid residue |
| (SEQ ID NO: 52) | (SEQ ID NO: 53) |
| 18 | F |
| 20 | N |
| 26 | S |
| 69 | — (deletion) |
| 70 | — (deletion) |
| 144 | — (deletion) |
| 417 | N |
| 484 | K |
| 501 | Y |
| 614 | G |
| 681 | H |
| 986 | P |
| 987 | P |

Optionally a polypeptide of the invention comprising an amino acid sequence of SEQ ID NO: 53, or an amino acid sequence which has at least 99% amino acid identity over its entire length with the amino acid sequence of SEQ ID NO:53, comprises at least five of the amino acid residues or deletions, at positions corresponding to the amino acid residue positions of SEQ ID NO:52, as shown in Table 9.4.

Optionally a polypeptide of the invention comprising an amino acid sequence of SEQ ID NO: 53, or an amino acid sequence which has at least 99% amino acid identity over its entire length with the amino acid sequence of SEQ ID NO:53, comprises at least ten of the amino acid residues or deletions, at positions corresponding to the amino acid residue positions of SEQ ID NO:52, as shown in Table 9.4.

Optionally a polypeptide of the invention comprising an amino acid sequence of SEQ ID NO: 53, or an amino acid sequence which has at least 99% amino acid identity over its entire length with the amino acid sequence of SEQ ID NO:53, comprises amino acid residue P at position 986, and amino acid residue P at position 987, corresponding to the amino acid residue positions of SEQ ID NO:52, and at least one, or all of the amino acid residues or deletions, at positions corresponding to the amino acid residue positions of SEQ ID NO:52, as shown in Table 9.5 below:

TABLE 9.5

| SARS2 S protein | COV_S_T2_29 |
|---|---|
| residue position | amino acid residue |
| (SEQ ID NO: 52 | (SEQ ID NO: 53) |
| 18 | F |
| 20 | N |
| 26 | S |
| 69 | — (deletion) |
| 70 | — (deletion) |
| 144 | — (deletion) |
| 417 | N |
| 484 | K |
| 501 | Y |
| 614 | G |
| 681 | H |

According to the invention there is also provided an isolated polypeptide which comprises a coronavirus S protein with at least one, or all of the amino acid residues or deletions, at positions corresponding to the amino acid residue positions of SEQ ID NO:52, as shown in Table 9.4 above.

Optionally an isolated polypeptide of the invention which comprises at least one of the amino acid residues or deletions, at positions corresponding to the amino acid residue positions of SEQ ID NO:52, as shown in Table 9.4 above, comprises at least five of the amino acid residues or deletions, at positions corresponding to the amino acid residue positions of SEQ ID NO: 52, as shown in Table 9.4 above.

Optionally an isolated polypeptide of the invention which comprises at least one of the amino acid residues or deletions, at positions corresponding to the amino acid residue positions of SEQ ID NO:52, as shown in Table 9.4 above, comprises at least ten of the amino acid residues or deletions, at positions corresponding to the amino acid residue positions of SEQ ID NO:52, as shown in Table 9.4 above.

Optionally the coronavirus S protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:52.

Optionally an isolated polypeptide of the invention which comprises at least one of the amino acid residues or deletions, at positions corresponding to the amino acid residue positions of SEQ ID NO:52, as shown in Table 9.4 above, comprises amino acid residue P at position 986, and amino acid residue P at position 987, corresponding to the amino acid residue positions of SEQ ID NO:52, and at least one, or all of the amino acid residues or deletions, at positions corresponding to the amino acid residue positions of SEQ ID NO:52, as shown in Table 9.5 above.

As described in more detail in Example 37 below, we have designed new full-length S protein COV_S_T2_29 with an arginine residue at position 498 of SEQ ID NO:52 (COV_S_T2_29+Q498R; SEQ ID NO:87), which corresponds to position 495, of SEQ ID NO:53 (COV_S_T2_29). The designed construct is effective for use as a COVID-19 vaccine insert to protect against variants B.1.617.2, P.1, B.1.351, and BA. 1, as explained in the Example. The amino acid sequence of the designed full-length S protein sequence is given below, and in Example 37:

>COV_S_T2_29 + Q498R
(SEQ ID NO: 87)
MFVFLVLLPLVSSQCVNFTNRTQLPSAYTNSFTRGVYYPDKVFRSSVLHSTQDLFLPFFSNVTWFHAI--SG
TNGTKRFDNPVLPFNDGVYFASTEKSNIIRGWIFGTTLDSKTQSLLIVNNATNVVIKVCEFQFCNDPFLGV-
YHKNNKSWMESEFRVYSSANNCTFEYVSQPFLMDLEGKQGNFKNLREFVFKNIDGYFKIYSKHTPINLVR--
-DLPQGFSALEPLVDLPIGINITRFQTLLALHRSYLTPGDSSSGWTAGAAAYYVGYLQPRTFLLKYNENGTI
TDAVDCALDPLSETKCTLKSFTVEKGIYQTSNFRVQPTESIVRFPNITNLCPFGEVFNATRFASVYAWNRKR
ISNCVADYSVLYNSASFSTFKCYGVSPTKLNDLCFTNVYADSFVIRGDEVRQIAPGQTGNIADYNYKLPDDF
TGCVIAWNSNNLDSKVGGNYNYLYRLFRKSNLKPFERDISTEIYQAGSTPCNGVKGFNCYFPLQSYGFRPTY
GVGYQPYRVVVLSFELLHAPATVCGPKKSTNLVKNKCVNFNFNGLTGTGVLTESNKKFLPFQQFGRDIADTT
DAVRDPQTLEILDITPCSFGGVSVITPGTNTSNQVAVLYQGVNCTEVPVAIHADQLTPTWRVYSTGSNVFQT
RAGCLIGAEHVNNSYECDIPIGAGICASYQTQTNSHRRARSVASQSIIAYTMSLGAENSVAYSNNSIAIPTN
FTISVTTEILPVSMTKTSVDCTMYICGDSTECSNLLLQYGSFCTQLNRALTGIAVEQDKNTQEVFAQVKQIY
KTPPIKDFGGFNFSQILPDPSKPSKRSFIEDLLFNKVTLADAGFIKQYGDCLGDIAARDLICAQKFNGLTVL
PPLLTDEMIAQYTSALLAGTITSGWTFGAGAALQIPFAMQMAYRFNGIGVTQNVLYENQKLIANQFNSAIGK
IQDSLSSTASALGKLQDVVNQNAQALNTLVKQLSSNFGAISSVLNDILSRLDPPEAEVQIDRLITGRLQSLQ
TYVTQQLIRAAEIRASANLAATKMSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNCDVVIGIVNNTVYDPLQPELDSFK
EELDKYFKNHTSPDVDLGDISGINASVVNIQKEIDRLNEVAKNLNESLIDLQELGKYEQYIKWPWYIWLGFI
AGLIAIVMVTIMLCCMTSCCSCLKGCCSCGSCCKFDEDDSEPVLKGVKLHYT

The amino acid differences of the designed sequence COV_S_T2_29+Q498R (SEQ ID NO:87) from the SARS2 reference sequence (SEQ ID NO:52) are shown in Table 9.6 below:

TABLE 9.6

| SARS2 S protein | SARS2 Reference | COV_S_T2_29 + Q498R |
|---|---|---|
| residue position | amino acid residue | amino acid residue |
| (SEQ ID NO: 52) | (SEQ ID NO: 52) | (SEQ ID NO: 87) |
| 18 | L | F |
| 20 | T | N |
| 26 | P | S |
| 69 | H | — (deletion) |
| 70 | V | — (deletion) |
| 144 | Y | — (deletion) |
| 417 | K | N |
| 484 | E | K |
| 498 | Q | R |
| 501 | N | Y |
| 614 | D | G |
| 681 | P | H |
| 986 | K | P |
| 987 | V | P |

According to the invention there is provided an isolated polypeptide, which comprises an amino acid sequence of SEQ ID NO:87.

According to the invention there is also provided an isolated polypeptide comprising an amino acid sequence of SEQ ID NO:87, or an amino acid sequence which has at least 99% amino acid identity over its entire length with the amino acid sequence of SEQ ID NO:87.

Optionally a polypeptide of the invention comprising an amino acid sequence of SEQ ID NO: 87, or an amino acid sequence which has at least 99% amino acid identity over its entire length with the amino acid sequence of SEQ ID NO:87, comprises at least one, or all of the amino acid residues or deletions, at positions corresponding to the amino acid residue positions of SEQ ID NO:52, as shown in Table 9.7 below:

TABLE 9.7

| SARS2 S protein | COV_S_T2_29 + Q498R |
|---|---|
| residue position | amino acid residue |
| (SEQ ID NO: 52) | (SEQ ID NO: 87) |
| 18 | F |
| 20 | N |
| 26 | S |
| 69 | — (deletion) |
| 70 | — (deletion) |
| 144 | — (deletion) |
| 417 | N |
| 484 | K |
| 498 | R |
| 501 | Y |
| 614 | G |
| 681 | H |
| 986 | P |
| 987 | P |

Optionally a polypeptide of the invention comprising an amino acid sequence of SEQ ID NO: 87, or an amino acid sequence which has at least 99% amino acid identity over its entire length with the amino acid sequence of SEQ ID NO:87, comprises at least five of the amino acid residues or deletions, at positions corresponding to the amino acid residue positions of SEQ ID NO:52, as shown in Table 9.7.

Optionally a polypeptide of the invention comprising an amino acid sequence of SEQ ID NO: 87, or an amino acid sequence which has at least 99% amino acid identity over its entire length with the amino acid sequence of SEQ ID NO:87, comprises at least ten of the amino acid residues or deletions, at positions corresponding to the amino acid residue positions of SEQ ID NO:52, as shown in Table 9.7.

Optionally a polypeptide of the invention comprising an amino acid sequence of SEQ ID NO: 87, or an amino acid sequence which has at least 99% amino acid identity over its entire length with the amino acid sequence of SEQ ID NO:87, comprises amino acid residue P at position 986, and amino acid residue P at position 987, corresponding to the amino acid residue positions of SEQ ID NO:52, and at least one, or all of the amino acid residues or deletions, at positions corresponding to the amino acid residue positions of SEQ ID NO:52, as shown in Table 9.8 below:

TABLE 9.8

| SARS2 S protein | COV_S_T2_29 + Q498R |
|---|---|
| residue position | amino acid residue |
| (SEQ ID NO: 52) | (SEQ ID NO: 87) |
| 18 | F |
| 20 | N |
| 26 | S |
| 69 | — (deletion) |
| 70 | — (deletion) |
| 144 | — (deletion) |
| 417 | N |
| 484 | K |
| 498 | R |
| 501 | Y |
| 614 | G |
| 681 | H |

According to the invention there is also provided an isolated polypeptide which comprises a coronavirus S protein with at least one, or all of the amino acid residues or deletions, at positions corresponding to the amino acid residue positions of SEQ ID NO:52, as shown in Table 9.8 above.

The designed construct is effective for use as a COVID-19 vaccine insert to protect against variants B.1.617.2, P.1, B.1.351, and BA.1 (delta, gamma, beta, and omicron BA.1, respectively), as explained in the Example. Also as explained in Example 37, the designed construct generated at least two-fold better neutralising response against Beta, Gamma, and Omicron in comparison to WTdER (FIG. 50C) after three doses of DNA vaccine. The neutralising antibody titres against Delta challenge were lower than WTdER (FIG. 50C) before MVA boost.

As described in more detail in Example 37 below, we have also designed new full-length S protein COV_S_T2_29+Q498R with 19 amino acid C-terminal truncation (dER) (COV_S_T2_29+Q498R+dER; SEQ ID NO:88). The designed construct is effective for use as a COVID-19 vaccine insert to protect against variants B.1.617.2, P.1, B.1.351, and BA.1, as explained in the Example. The amino acid sequence of the designed full-length S protein sequence is given below, and in Example 37:

>COV_S_T2_29 + Q498R + dER
(SEQ ID NO: 88)
MFVFLVLLPLVSSQCVNFTNRTQLPSAYTNSFTRGVYYPDKVFRSSVLHSTQDLFLPFFSNVTWFHAI--SG
TNGTKRFDNPVLPFNDGVYFASTEKSNIIRGWIFGTTLDSKTQSLLIVNNATNVVIKVCEFQFCNDPFLGV-
YHKNNKSWMESEFRVYSSANNCTFEYVSQPFLMDLEGKQGNFKNLREFVFKNIDGYFKIYSKHTPINLVR--
-DLPQGFSALEPLVDLPIGINITRFQTLLALHRSYLTPGDSSSGWTAGAAAYYVGYLQPRTFLLKYNENGTI
TDAVDCALDPLSETKCTLKSFTVEKGIYQTSNFRVQPTESIVRFPNITNLCPFGEVFNATRFASVYAWNRKR
ISNCVADYSVLYNSASFSTFKCYGVSPTKLNDLCFTNVYADSFVIRGDEVRQIAPGQTGNIADYNYKLPDDF
TGCVIAWNSNNLDSKVGGNYNYLYRLFRKSNLKPFERDISTEIYQAGSTPCNGVKGFNCYFPLQSYGFRPTY
GVGYQPYRVVVLSFELLHAPATVCGPKKSTNLVKNKCVNFNFNGLTGTGVLTESNKKFLPFQQFGRDIADTT
DAVRDPQTLEILDITPCSFGGVSVITPGTNTSNQVAVLYQGVNCTEVPVAIHADQLTPTWRVYSTGSNVFQT
RAGCLIGAEHVNNSYECDIPIGAGICASYQTQTNSHRRARSVASQSIIAYTMSLGAENSVAYSNNSIAIPTN
FTISVTTEILPVSMTKTSVDCTMYICGDSTECSNLLLQYGSFCTQLNRALTGIAVEQDKNTQEVFAQVKQIY
KTPPIKDFGGFNFSQILPDPSKPSKRSFIEDLLFNKVTLADAGFIKQYGDCLGDIAARDLICAQKFNGLTVL
PPLLTDEMIAQYTSALLAGTITSGWTFGAGAALQIPFAMQMAYRFNGIGVTQNVLYENQKLIANQFNSAIGK
IQDSLSSTASALGKLQDVVNQNAQALNTLVKQLSSNFGAISSVLNDILSRLDPPEAEVQIDRLITGRLQSLQ
TYVTQQLIRAAEIRASANLAATKMSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNCDVVIGIVNNTVYDPLQPELDSFK
EELDKYFKNHTSPDVDLGDISGINASVVNIQKEIDRLNEVAKNLNESLIDLQELGKYEQYIKWPWYIWLGFI
AGLIAIVMVTIMLCCMTSCCSCLKGCCSCGSCC-

The amino acid differences of the designed sequence COV_S_T2_29+Q498R+dER (SEQ ID NO: 88) from the SARS2 reference sequence (SEQ ID NO:52) are shown in Table 9.9 below:

TABLE 9.9

| SARS2 S protein | SARS2 Reference | COV_S_T2_29 + Q498R + dER |
|---|---|---|
| residue position | amino acid residue | amino acid residue |
| (SEQ ID NO: 52) | (SEQ ID NO: 52) | (SEQ ID NO: 88) |
| 18 | L | F |
| 20 | T | N |
| 26 | P | S |
| 69 | H | — (deletion) |
| 70 | V | — (deletion) |
| 144 | Y | — (deletion) |
| 417 | K | N |
| 484 | E | K |
| 498 | Q | R |
| 501 | N | Y |
| 614 | D | G |
| 681 | P | H |
| 986 | K | P |
| 987 | V | P |
| 1255-1273 | GSCCKFDEDDSEPVLKGVKL | — (deletion) |
| | HYT | |

According to the invention there is provided an isolated polypeptide, which comprises an amino acid sequence of SEQ ID NO:88.

According to the invention there is also provided an isolated polypeptide comprising an amino acid sequence of SEQ ID NO:88, or an amino acid sequence which has at least 98% amino acid identity over its entire length with the amino acid sequence of SEQ ID NO:88.

Optionally a polypeptide of the invention comprising an amino acid sequence of SEQ ID NO: 88, or an amino acid sequence which has at least 98% amino acid identity over its entire length with the amino acid sequence of SEQ ID NO:88, comprises at least one, or all of the amino acid residues or deletions, at positions corresponding to the amino acid residue positions of SEQ ID NO:52, as shown in Table 9.10 below:

TABLE 9.10

| SARS2 S protein | COV_S_T2_29 + Q498R + dER |
|---|---|
| residue position | amino acid residue |
| (SEQ ID NO: 52) | (SEQ ID NO: 88) |
| 18 | F |
| 20 | N |
| 26 | S |
| 69 | — (deletion) |
| 70 | — (deletion) |
| 144 | — (deletion) |
| 417 | N |
| 484 | K |
| 498 | R |
| 501 | Y |
| 614 | G |
| 681 | H |
| 986 | P |
| 987 | P |
| 1255-1273 | — (deletion) |

Optionally a polypeptide of the invention comprising an amino acid sequence of SEQ ID NO: 88, or an amino acid sequence which has at least 98% amino acid identity over its entire length with the amino acid sequence of SEQ ID NO:88, comprises at least five of the amino acid residues or deletions, at positions corresponding to the amino acid residue positions of SEQ ID NO:52, as shown in Table 9.10.

Optionally a polypeptide of the invention comprising an amino acid sequence of SEQ ID NO: 88, or an amino acid sequence which has at least 98% amino acid identity over its entire length with the amino acid sequence of SEQ ID NO:88, comprises at least ten of the amino acid residues or deletions, at positions corresponding to the amino acid residue positions of SEQ ID NO:52, as shown in Table 9.10.

Optionally a polypeptide of the invention comprising an amino acid sequence of SEQ ID NO: 88, or an amino acid sequence which has at least 98% amino acid identity over its entire length with the amino acid sequence of SEQ ID NO:88, comprises at least fifteen of the amino acid residues or deletions, at positions corresponding to the amino acid residue positions of SEQ ID NO:52, as shown in Table 9.10.

Optionally a polypeptide of the invention comprising an amino acid sequence of SEQ ID NO: 88, or an amino acid sequence which has at least 98% amino acid identity over its entire length with the amino acid sequence of SEQ ID NO:88, comprises amino acid residue P at position 986, and amino acid residue P at position 987, corresponding to the amino acid residue positions of SEQ ID NO:52, and at least one, or all of the amino acid residues or deletions, at positions corresponding to the amino acid residue positions of SEQ ID NO:52, as shown in Table 9.11 below:

TABLE 9.11

| SARS2 S protein | COV_S_T2_29 + Q498R + dER |
|---|---|
| residue position | amino acid residue |
| (SEQ ID NO: 52) | (SEQ ID NO: 88) |
| 18 | F |
| 20 | N |
| 26 | S |
| 69 | — (deletion) |
| 70 | — (deletion) |
| 144 | — (deletion) |
| 417 | N |
| 484 | K |
| 498 | R |
| 501 | Y |
| 614 | G |
| 681 | H |
| 1255-1273 | — (deletion) |

According to the invention there is also provided an isolated polypeptide which comprises a coronavirus S protein with at least one, or all of the amino acid residues or deletions, at positions corresponding to the amino acid residue positions of SEQ ID NO:52, as shown in Table 9.11 above.

The designed construct is effective for use as a COVID-19 vaccine insert to protect against variants B.1.617.2, P.1, B.1.351, and BA.1 (delta, gamma, beta, and omicron, respectively), as explained in the Example. Also as explained in Example 37, the designed construct generated at least two-fold better neutralising response against Beta, Gamma, and Omicron in comparison to WTdER (FIG. 50C) after three doses of DNA vaccine. The neutralising antibody titres against both the Ancestral sequence and Delta were comparable to WTdER (FIG. 50C) for the T2_29+Q+dER design.

### Designed Coronavirus S Protein Sequence in Closed State to Protect Against COVID-19 Variants, and Predicted Future Variants

The majority of SARS-CoV-2 vaccines in use or in advanced clinical development are based on the viral spike protein(S) as their immunogen. S is present on virions as pre-fusion trimers in which the receptor binding domain (RBD) is stochastically open or closed. Neutralizing antibodies have been described that act against both open and closed conformations. The long-term success of vaccination strategies will depend upon inducing antibodies that provide long-lasting broad immunity against evolving, circulating SARS-CoV-2 strains, while avoiding the risk of antibody dependent enhancement as observed with other Coronavirus vaccines.

Carnell et al. (“SARS-CoV-2 spike protein arrested in the closed state induces potent neutralizing responses”; https://doi.org/10.1101/2021.01.14.426695, posted 14 Jan. 2021) have assessed the results of immunization in a mouse model using an S protein trimer that is arrested in the closed state to prevent exposure of the receptor binding site and therefore interaction with the receptor. The authors compared this with a range of other modified S protein constructs, including representatives used in current vaccines. They found that all trimeric S proteins induce a long-lived, strongly neutralizing antibody response as well as T-cell responses. Notably, the protein binding properties of sera induced by the closed spike differed from those induced by standard S protein constructs. Closed S proteins induced more potent neutralising responses than expected based on the degree to which they inhibit interactions between the RBD and ACE2. The authors conclude that these observations suggest that closed spikes recruit different, but equally potent, virus-inhibiting immune responses than open spikes, and that this is likely to include neutralizing antibodies against conformational epitopes present in the closed conformation.

We have appreciated that the amino acid changes of the designed S protein sequences disclosed herein (and especially of SEQ ID NO:53 as described in Example 30) may optionally be present in a designed S protein that is arrested in the closed state, and thereby further improve the antibody response of the designed sequences. In particular, use of such structural constraints may reduce immunodominance to key regions, and spread the antibody response to focus on other, or less immunodominant sites.

Example 31 below describes optional additional amino acid changes that may be made to a designed S protein sequence to allow it to form a closed structure.

Optionally a designed S protein sequence of the invention may comprise cysteine residues at positions corresponding to positions 413 and 987 of the full length S protein sequence. For example, G413C and V987C.

For example, a designed S protein sequence of the invention may comprise the following amino acid sequence (SEQ ID NO:54) (with cysteine residues at positions 410 and 984, which correspond to positions 413 and 987, respectively, of SEQ ID NO:52):

| | |
|---|---|
| MFVFLVLLPL VSSQCVNFTN RTQLPSAYTN SFTRGVYYPD KVFRSSVLHS TQDLFLPFFS | 60 |
| NVTWFHAISG TNGTKRFDNP VLPFNDGVYF ASTEKSNIIR GWIFGTTLDS KTQSLLIVNN | 120 |
| ATNVVIKVCE FQFCNDPFLG VYHKNNKSWM ESFFRVYSSA NNCTFEYVSQ PFLMDLEGKQ | 180 |
| GNFKNLREFV FKNIDGYFKI YSKHTPINLV RDLPQGFSAL EPLVDLPIGI NITRFQTLLA | 240 |
| LHRSYLTPGD SSSGWTAGAA AYYVGYLQPR TFLLKYNENG TITDAVDCAL DPLSETKCTL | 300 |
| KSFTVEKGIY QTSNFRVQPT ESIVRFPNIT NLCPFGEVFN ATRFASVYAW NRKRISNCVA | 360 |
| DYSVLYNSAS FSTFKCYGVS PTKLNDLCFT NVYADSFVIR GDEVRQIAPC QTGNIADYNY | 420 |
| KLPDDFTGCV IAWNSNNLDS KVGGNYNYLY RLFRKSNLKP FERDISTEIY QAGSTPCNGV | 480 |
| KGFNCYFPLQ SYGFQPTYGV GYQPYRVVVL SFELLHAPAT VCGPKKSTNL VKNKCVNFNF | 540 |
| NGLTGTGVLT ESNKKFLPFQ QFGRDIADTT DAVRDPQTLE ILDITPCSFG GVSVITPGTN | 600 |
| TSNQVAVLYQ GVNCTEVPVA IHADQLTPTW RVYSTGSNVF QTRAGCLIGA EHVNNSYECD | 660 |
| IPIGAGICAS YQTQTNSHRR ARSVASQSII AYTMSLGAEN SVAYSNNSIA IPTNFTISVT | 720 |
| TEILPVSMTK TSVDCTMYIC GDSTECSNLL LQYGSFCTQL NRALTGIAVE QDKNTQEVFA | 780 |
| QVKQIYKTPP IKDFGGFNFS QILPDPSKPS KRSFIEDLLF NKVTLADAGF IKQYGDCLGD | 840 |
| IAARDLICAQ KFNGLTVLPP LLTDEMIAQY TSALLAGTIT SGWTFGAGAA LQIPFAMQMA | 900 |
| YRFNGIGVTQ NVLYENQKLI ANQFNSAIGK IQDSLSSTAS ALGKLQDVVN QNAQALNTLV | 960 |
| KQLSSNFGAI SSVLNDILSR LDPCEAEVQI DRLITGRLQS LQTYVTQQLI RAAEIRASAN | 1020 |
| LAATKMSECV LGQSKRVDFC GKGYHLMSFP QSAPHGVVFL HVTYVPAQEK NFTTAPAICH | 1080 |
| DGKAHFPREG VFVSNGTHWF VTQRNFYEPQ IITTDNTFVS GNCDVVIGIV NNTVYDPLQP | 1140 |
| ELDSFKEELD KYFKNHTSPD VDLGDISGIN ASVVNIQKEI DRLNEVAKNL NESLIDLQEL | 1200 |
| GKYEQYIKWP WYIWLGFIAG LIAIVMVTIM LCCMTSCCSC LKGCCSCGSC CKFDEDDSEP | 1260 |
| VLKGVKLHYT | 1270 |

According to the invention there is provided an isolated polypeptide, which comprises an amino acid sequence of SEQ ID NO:54.

According to the invention there is also provided an isolated polypeptide comprising an amino acid sequence of SEQ ID NO:54, or an amino acid sequence which has at least 99% amino acid identity over its entire length with the amino acid sequence of SEQ ID NO:54.

Optionally a polypeptide of the invention comprising an amino acid sequence of SEQ ID NO: 54, or an amino acid sequence which has at least 99% amino acid identity over its entire length with the amino acid sequence of SEQ ID NO:54, comprises at least one, or all of the amino acid residues or deletions, at positions corresponding to the amino acid residue positions of SEQ ID NO:52, as shown in Table 9.4 below:

TABLE 9.4

| SARS2 S protein | COV_S_T2_29 |
|---|---|
| residue position | amino acid residue |
| (SEQ ID NO: 52) | (SEQ ID NO: 53) |
| 18 | F |
| 20 | N |
| 26 | S |
| 69 | — (deletion) |
| 70 | — (deletion) |
| 144 | — (deletion) |
| 417 | N |
| 484 | K |
| 501 | Y |
| 614 | G |
| 681 | H |
| 986 | P |
| 987 | P |

Optionally a polypeptide of the invention comprising an amino acid sequence of SEQ ID NO: 54, or an amino acid sequence which has at least 99% amino acid identity over its entire length with the amino acid sequence of SEQ ID NO:54, comprises at least five of the amino acid residues or deletions, at positions corresponding to the amino acid residue positions of SEQ ID NO:52, as shown in Table 9.4.

Optionally a polypeptide of the invention comprising an amino acid sequence of SEQ ID NO: 54, or an amino acid sequence which has at least 99% amino acid identity over its entire length with the amino acid sequence of SEQ ID NO:54, comprises at least ten of the amino acid residues or deletions, at positions corresponding to the amino acid residue positions of SEQ ID NO:52, as shown in Table 9.4.

Optionally a polypeptide of the invention comprising an amino acid sequence of SEQ ID NO: 54, or an amino acid sequence which has at least 99% amino acid identity over its entire length with the amino acid sequence of SEQ ID NO:54, comprises at least one, or all of the amino acid residues or deletions, at positions corresponding to the amino acid residue positions of SEQ ID NO:52, as shown in Table 9.5 below:

Optionally a polypeptide of the invention comprising an amino acid sequence of SEQ ID NO: 54, or an amino acid sequence which has at least 99% amino acid identity over its entire length with the amino acid sequence of SEQ ID NO:54, comprises amino acid residue P at position 986 corresponding to the amino acid residue positions of SEQ ID NO:52, and at least one, or all of the amino acid residues or deletions, at positions corresponding to the amino acid residue positions of SEQ ID NO:52, as shown in Table 9.5 below:

TABLE 9.5

| SARS2 S protein | COV_S_T2_29 |
|---|---|
| residue position | amino acid residue |
| (SEQ ID NO: 52) | (SEQ ID NO: 53) |
| 18 | F |
| 20 | N |
| 26 | S |
| 69 | — (deletion) |
| 70 | — (deletion) |
| 144 | — (deletion) |
| 417 | N |
| 484 | K |
| 501 | Y |
| 614 | G |
| 681 | H |

According to the invention there is also provided an isolated polypeptide which comprises a coronavirus S protein comprising cysteine amino acid residues at positions corresponding to positions 413 and 987 of SEQ ID NO:52, and at least one, or all of the amino acid residues or deletions, at positions corresponding to the amino acid residue positions of SEQ ID NO:52, as shown in Table 9.5 above.

Optionally an isolated polypeptide of the invention which comprises cysteine amino acid residues at positions corresponding to positions 413 and 987 of SEQ ID NO:52, and at least one of the amino acid residues or deletions, at positions corresponding to the amino acid residue positions of SEQ ID NO:52, as shown in Table 9.5 above, comprises at least five of the amino acid residues or deletions, at positions corresponding to the amino acid residue positions of SEQ ID NO:52, as shown in Table 9.5 above.

Optionally an isolated polypeptide of the invention which comprises cysteine amino acid residues at positions corresponding to positions 413 and 987 of SEQ ID NO:52, and at least one of the amino acid residues or deletions, at positions corresponding to the amino acid residue positions of SEQ ID NO:52, as shown in Table 9.5 above, comprises at least ten of the amino acid residues or deletions, at positions corresponding to the amino acid residue positions of SEQ ID NO:52, as shown in Table 9.5 above.

Optionally an isolated polypeptide of the invention which comprises cysteine amino acid residues at positions corresponding to positions 413 and 987 of SEQ ID NO:52, and at least one of the amino acid residues or deletions, at positions corresponding to the amino acid residue positions of SEQ ID NO:52, as shown in Table 9.5 above, comprises amino acid residue P at position 986.

We have also appreciated that any SARS-CoV-2 spike protein may be modified to include cysteine residues at positions corresponding to positions 413 and 987 of SEQ ID NO:52 to allow it to form a spike protein arrested in the closed state, in accordance with Carnell et al. (supra), and thereby elicit more potent neutralising responses compared with the corresponding unmodified protein. For example, Jeong et al. (https://virological.org/t/assemblies-of-putative-sars-cov2-spike-encoding-mma-sequences-for-vaccines-bnt-162b2-and-mma-1273/663—version 0.2Beta Mar. 30, 2021) have recently reported experimental sequence information for the RNA components of the initial Moderna (https://pubmed.ncbi.nlm.nih.gov/32756549/) and Pfizer/BioNTech (https://pubmed.ncbi.nlm.nih.gov/33301246/) COVID-19 vaccines, allowing a working assembly of the former and a confirmation of previously reported sequence information for the latter RNA (see the sequences provided in FIGS. 1 and 2 of the document). Spike protein encoded by such sequence may be modified to include cysteine residues at positions corresponding to positions 413 and 987 of SEQ ID NO:52.

According to the invention there is also provided an isolated polypeptide which comprises a coronavirus S protein comprising cysteine amino acid residues at positions corresponding to positions 413 and 987 of SEQ ID NO:52.

Optionally the coronavirus S protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:52.

SARS-CoV-2 is continually evolving, with more contagious mutations spreading rapidly. Zahradník et al., 2021 (“SARS-CoV-2 RBD in vitro evolution follows contagious mutation spread, yet generates an able infection inhibitor”; doi: https://doi.org/10.1101/2021.01.06.425392, posted 29 Jan. 2021) recently reported using in vitro evolution to affinity maturate the receptor-binding domain (RBD) of the spike protein towards ACE2 resulting in the more contagious mutations, S477N, E484K, and N501Y, to be among the first selected, explaining the convergent evolution of the “European” (20E-EU1), “British” (501.V1), “South African” (501.V2), and “Brazilian” variants (501.V3). The authors report that further in vitro evolution enhancing binding by 600-fold provides guidelines towards potentially new evolving mutations with even higher infectivity. For example, Q498R epistatic to N501Y.

We have also appreciated that the designed S protein sequences (RBD, truncated, or full-length) disclosed herein (and especially in the sections entitled “Designed Coronavirus full-length S protein sequence to protect against COVID-19 variants”, and “Designed Coronavirus S protein sequence in closed state to protect against COVID-19 variants, and predicted future variants” above, and in Examples 30 and 31 below) may optionally also include amino acid substitutions at one or more residue positions predicted to be mutated in future COVID-19 variants with a vaccine escape response, for example at one or more (or all) of positions 446, 452, 477, and 498 (for example, G446R, S477N, Q498R, especially Q498R).

Optionally an isolated polypeptide of the invention includes amino acid changes at one or more (or all) of the following positions (corresponding to amino acid residue positions of SEQ ID NO: 52): 446, 452, 477, and 498 (for example, G446R, S477N, Q498R, especially Q498R).

Optionally an isolated polypeptide of the invention includes amino acid changes at positions (corresponding to amino acid residue positions of SEQ ID NO:52): Q498R and N501Y.

### Designed Coronavirus Envelope (E) Protein Sequences

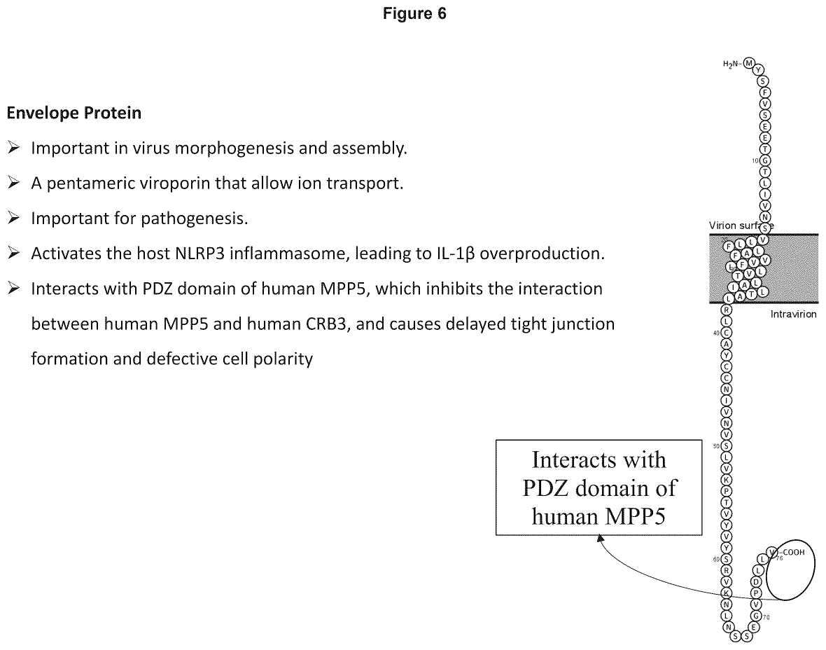

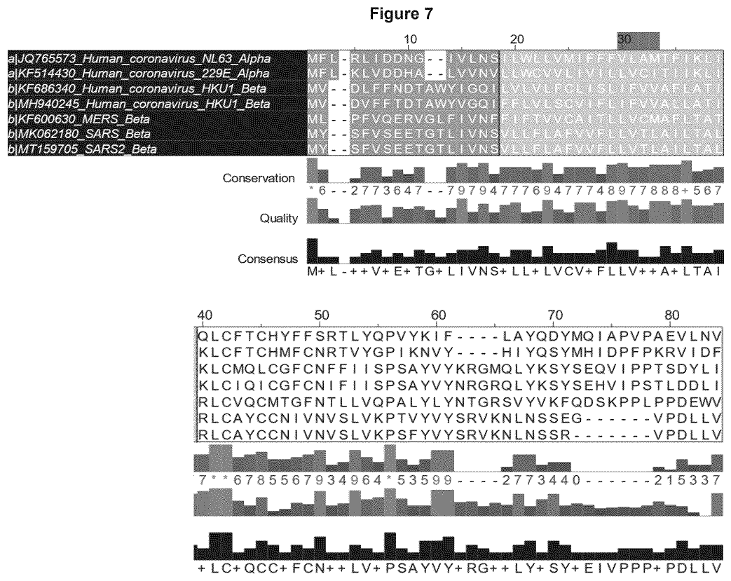

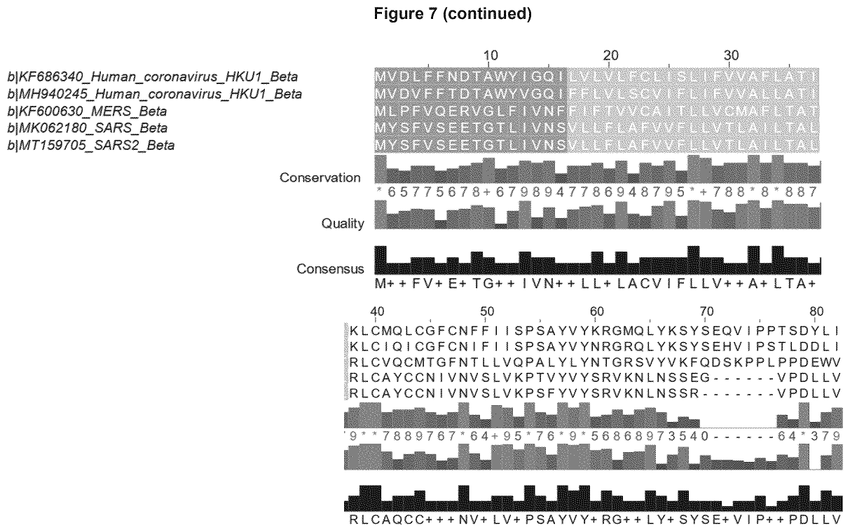

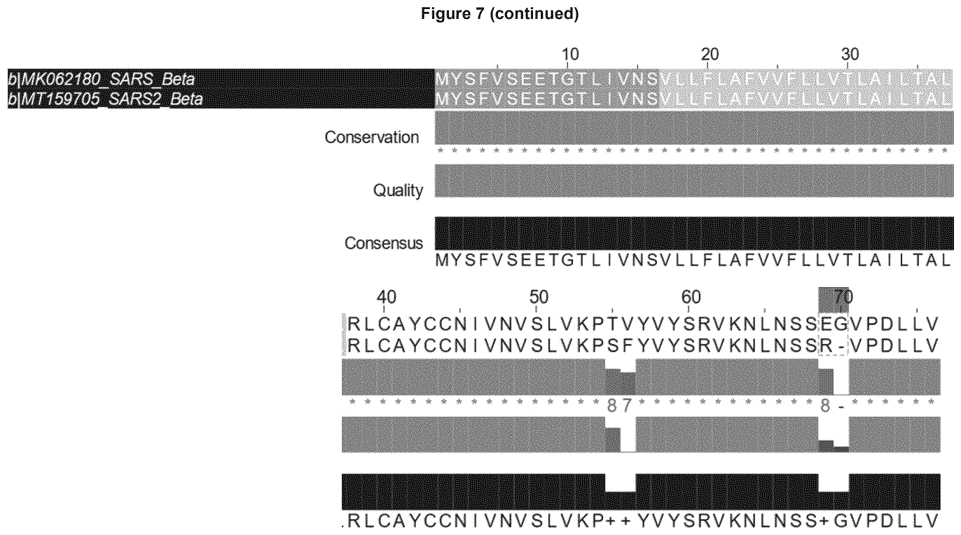

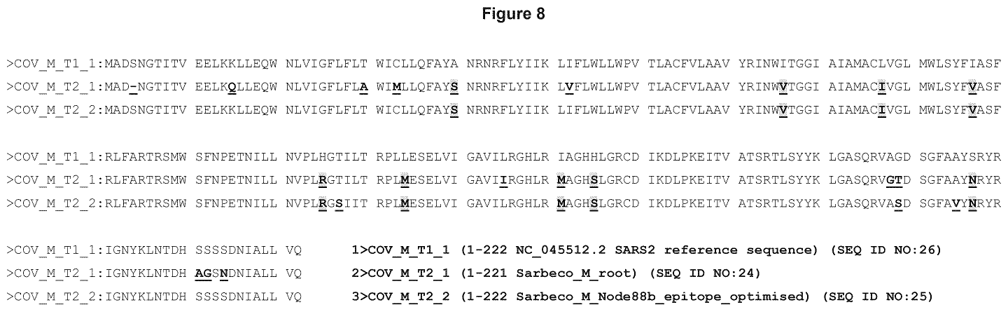

We have also generated novel amino acid sequences for coronavirus Envelope (E) protein. FIG. 6 shows an amino acid sequence of the SARS Envelope (E) protein (SEQ ID NO:21), and illustrates key features of the sequence. As described in Example 10 below, FIG. 7 shows a multiple sequence alignment of coronavirus E protein sequences, comparing sequences for isolates of NL63 and 229E (alpha-coronaviruses), and HKU1, MERS, SARS, and SARS2 (beta-coronaviruses). The alignment shows that the C-terminal end of the E protein for the SARS2 and SARS sequences (beta-coronaviruses of subgenus Sarbeco) includes a deletion, compared with the other sequences, and that the SARS2 E protein sequence includes a deletion, and an Arginine (positively charged) amino acid residue, compared with the SARS sequence.

The novel amino acid sequences for coronavirus E protein are called COV_E_T2_1 (a designed Sarbecovirus sequence) (SEQ ID NO:22) and COV_E_T2_2 (a designed SARS2 sequence) (SEQ ID NO:23):

>COV_E_T2_1
(SEQ ID NO: 22)
MYSFVSEETG TLIVNSVLLF LAFVVFLLVT LAILTALRLC
AYCCNIVNVS LVKPTFYVYS RVKNLNSSQG VPDLLV
>COV_E_T2_2
(SEQ ID NO: 23)
MYSFVSEETG TLIVNSVLLF LAFVVFLLVT LAILTALRLC
AYCCNIVNVS LVKPTFYVYS RVKNLNSSR- VPDLLV

Alignment of the SARS2 reference E protein sequence in FIG. 7 with these designed sequences highlights that there are four amino acid differences between the SARS2 reference E protein sequence and the COV_E_T2_1 designed sequence (SEQ ID NO:22), and two amino acid differences between the SARS2 reference E protein sequence and the COV_E_T2_2 designed sequence (SEQ ID NO:23):

| | | |
|---|---|---|
| SARS2 COV_E_T2_1 COV_E_T2_2 | 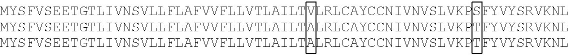 | 65 65 65 |
| SARS2 COV_E_T2_1 COV_E_T2_2 | 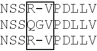 | 75 76 75 |

The C-terminal sequence of the COV_E_T2_2 sequence is identical to the SARS2 reference sequence. The C-terminal of the E protein is one of the identified epitopes for E-protein, so the amino acid deletion and the substitution with an Arginine residue present in the SARS2 reference sequence (compared with the SARS reference sequence in FIG. 6) have been retained in the COV_E_T2_2 designed sequence. The amino acid differences at the other positions are optimised to maximise induction of an immune response that recognises all Sarbeco viruses.

The amino acid differences are summarised in the table below:

TABLE 10.1

| SARS2 E | SARS2 | COV_E_T2_1 | COV_E_T2_2 |
|---|---|---|---|
| protein residue | Reference Amino | Amino acid | Amino acid |
| position | acid residue | residue | residue |
| 36 | V | A | A |
| 55 | S | T | T |
| 69 | R | Q | R |
| 70 | — | G | — |

There is also provided according to the invention an isolated polypeptide which comprises an amino acid sequence of SEQ ID NO:22, or an amino acid sequence which has at least 95%, 96%, 97%, 98%, or 99% amino acid identity over its entire length with the amino acid sequence of SEQ ID NO:22.

Optionally an isolated polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, comprises one or both amino acid residues, at positions corresponding to the amino acid residue positions of SEQ ID NO:22, as shown in the table below:

TABLE 10.2

| SARS2 E protein | COV_E_T2_1 Amino |
|---|---|
| residue position | acid residue |
| 36 | A |
| 55 | T |

Optionally an isolated polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, comprises any, at least two, at least three, or all, of the amino acid residues, at positions corresponding to the amino acid residue positions of SEQ ID NO:22, as shown in the table below:

TABLE 10.3

| SARS2 E protein | COV_E_T2_1 Amino |
|---|---|
| residue position | acid residue |
| 36 | A |
| 55 | T |
| 69 | Q |
| 70 | G |

There is also provided according to the invention an isolated polypeptide which comprises an amino acid sequence of SEQ ID NO:22.

There is also provided according to the invention an isolated polypeptide, which comprises an amino acid sequence of SEQ ID NO:23, or an amino acid sequence which has at least 98%, or 99% amino acid identity over its entire length with the amino acid sequence of SEQ ID NO: 23.

Optionally an isolated polypeptide of the invention which comprises an amino acid sequence of SEQ ID NO:23, or an amino acid sequence which has at least 98%, or 99% amino acid identity over its entire length with the amino acid sequence of SEQ ID NO:23, comprises one or both amino acid residues, at positions corresponding to the amino acid residue positions of SEQ ID NO:23, as shown in the table below:

TABLE 10.4

| SARS2 E protein | COV_E_T2_2 Amino |
|---|---|
| residue position | acid residue |
| 36 | A |
| 55 | T |

There is also provided according to the invention an isolated polypeptide, which comprises an amino acid sequence of SEQ ID NO:23.

According to the invention there is also provided an isolated polypeptide, which comprises a coronavirus E protein with one or both of the amino acid residues at positions corresponding to the amino acid residue positions as shown in the table below:

TABLE 10.5

| E protein residue position | Amino acid residue |
|---|---|
| 36 | A |
| 55 | T |

According to the invention there is also provided an isolated polypeptide, which comprises a coronavirus E protein with any, at least two, at least three, or all, of the amino acid residues at positions corresponding to the amino acid residue positions as shown in the table below:

TABLE 10.6

| E protein residue position | Amino acid residue |
|---|---|
| 36 | A |
| 55 | T |
| 69 | Q |
| 70 | G |

Optionally an isolated polypeptide of the invention which comprises a coronavirus E protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:21.

In the alignment above residue 36 of the SARS2 reference sequence is shown as V, but is actually A (as correctly shown in FIG. 7 and SEQ ID NO:21). Alignment of SEQ ID NO:21 with the designed sequences highlights that there are three amino acid differences between the alternative SARS2 reference E protein sequence and the COV_E_T2_1 designed sequence (SEQ ID NO:22), and one amino acid difference between the SARS2 reference E protein sequence and the COV_E_T2_2 designed sequence (SEQ ID NO:23):

| | | |
|---|---|---|
| SARS2 COV_E_T2_1 COV_E_T2_2 | 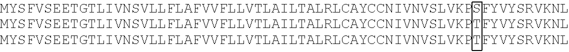 | 65 65 65 |
| SARS2 COV_E_T2_1 COV_E_T2_2 | 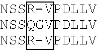 | 75 76 75 |

The amino acid differences are summarised in the table below:

TABLE 10.7

| SARS2 E | SARS2 | COV_E_T2_1 | COV_E_T2_2 |
|---|---|---|---|
| protein | Reference | Amino | Amino |
| residue | Amino acid | acid | acid |
| position | residue | residue | residue |
| 55 | S | T | T |
| 69 | R | Q | R |
| 70 | — | G | — |

There is also provided according to the invention an isolated polypeptide which comprises an amino acid sequence of SEQ ID NO:22 (COV_E_T2_1), or an amino acid sequence which has at least 97%, 98%, or 99% amino acid identity over its entire length with the amino acid sequence of SEQ ID NO:22.

Optionally an isolated polypeptide of the invention which comprises an amino acid sequence of SEQ ID NO:22, or an amino acid sequence which has at least 97%, 98%, or 99% amino acid identity over its entire length with the amino acid sequence of SEQ ID NO:22, comprises the amino acid residue, at a position corresponding to the amino acid residue position of SEQ ID NO: 22, as shown in the table below:

TABLE 10.8

| SARS2 E protein | COV_E_T2_1 Amino |
|---|---|
| residue position | acid residue |
| 55 | T |

Optionally an isolated polypeptide of the invention which comprises an amino acid sequence of SEQ ID NO:22, or an amino acid sequence which has at least 97%, 98%, or 99% amino acid identity over its entire length with the amino acid sequence of SEQ ID NO:22, comprises any, at least two, or all, of the amino acid residues, at positions corresponding to the amino acid residue positions of SEQ ID NO:22, as shown in the table below:

TABLE 10.9

| SARS2 E protein | COV_E_T2_1 Amino |
|---|---|
| residue position | acid residue |
| 55 | T |
| 69 | Q |
| 70 | G |

There is also provided according to the invention an isolated polypeptide, which comprises an amino acid sequence of SEQ ID NO:23, or an amino acid sequence which has at least 99% amino acid identity over its entire length with the amino acid sequence of SEQ ID NO:23.

Optionally an isolated polypeptide of the invention which comprises an amino acid sequence of SEQ ID NO:23, or an amino acid sequence which has at least 99% amino acid identity over its entire length with the amino acid sequence of SEQ ID NO:23, comprises an amino acid residues, at a position corresponding to the amino acid residue position a of SEQ ID NO:23, as shown in the table below:

TABLE 10.10

| SARS2 E protein | COV_E_T2_2 Amino |
|---|---|
| residue position | acid residue |
| 55 | T |

According to the invention there is also provided an isolated polypeptide, which comprises a coronavirus E protein with the amino acid residue at a position corresponding to the amino acid residue position as shown in the table below:

TABLE 10.11

| E protein residue | Amino acid |
|---|---|
| position | residue |
| 55 | T |

According to the invention there is also provided an isolated polypeptide, which comprises a coronavirus E protein with any, at least two, or all, of the amino acid residues at positions corresponding to the amino acid residue positions as shown in the table below:

TABLE 10.12

| E protein residue | Amino acid |
|---|---|
| position | residue |
| 55 | T |
| 69 | Q |
| 70 | G |

Optionally an isolated polypeptide of the invention which comprises a coronavirus E protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:21.

There is also provided according to the invention an isolated polypeptide, which comprises an amino acid sequence of SEQ ID NO:21.

SARS-CoV envelope (E) gene encodes a 76-amino acid transmembrane protein with ion channel (IC) activity, an important function in virus-host interaction. Infection of mice with viruses lacking or displaying E protein IC activity revealed that activation of the inflammasome pathway, and the exacerbated inflammatory response induced by SARS-CoV, was decreased in infections by ion channel-deficient viruses (Nieto-Torres et al., 2014, Severe Acute Respiratory Syndrome Coronavirus Envelope Protein on Channel Activity Promotes Virus Fitness and Pathogenesis. PLOS Pathog 10 (5): e1004077).

We have made new E protein designs Cov_E_T2_3, COV_E_T2_4 and CoV_E_T2_5, which correspond to new designs of SARS2 reference (SEQ ID NO:41), COV_E_T2_1 (SEQ ID NO:22), and CoV_E_T2_2 (SEQ ID NO:23) (see Example 10), respectively. These new designs have a point mutation, N15A, which abrogates the ion channel activity, but does not influence the stability of the structure. Nieto-Torres et al., supra, discusses this mutation as well as the toxicity and inflammatory action of SARS E on the host cell.

The amino acid sequence of SARS2 envelope protein reference (SEQ ID NO:41) is:

(SEQ ID NO: 41)
MYSFVSEETG TLIVNSVLLF LAFVVFLLVT LAILTALRLC
AYCCNIVNVS LVKPSFYVYS RVKNLNSSRV PDLLV

The amino acid sequences of the new E protein designs are shown below, and in Example 25:

>COV_E_T2_3 (SARS2_mutant)
(SEQ ID NO: 42)
MYSFVSEETG TLIVASVLLF LAFVVFLLVT LAILTALRLC
AYCCNIVNVS LVKPSFYVYS RVKNLNSSR- VPDLLV
>COV_E_T2_4 (Env1_mutant)
(SEQ ID NO: 43)
MYSFVSEETG TLIVASVLLF LAFVVFLLVT LAILTALRLC
AYCCNIVNVS LVKPTFYVYS RVKNLNSSQG VPDLLV
>COV_E_T2_5 (Env2_mutant)
(SEQ ID NO: 44)
MYSFVSEETG TLIVASVLLF LAFVVFLLVT LAILTALRLC
AYCCNIVNVS LVKPTFYVYS RVKNLNSSR- VPDLLV

Alignment of the E protein designs with SARS2 E protein reference sequence is shown below:

| | | |
|---|---|---|
| SARS2 COV_E_T2_1 COV_E_T2_2 COV_E_T2_3 COV_E_T2_4 COV_E_T2_5 | 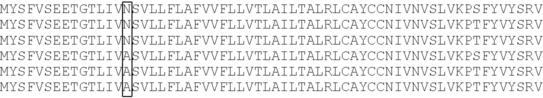 | 62 62 62 62 62 62 |
| SARS2 | 63 KNLNSSR-VPDLLV | 75 |
| COV_E_T2_1 | 63 KNLNSSQGVPDLLV | 75 |
| COV_E_T2_2 | 63 KNLNSSA-VPDLLV | 75 |
| COV_E_T2_3 | 63 KNLNSSR-VPDLLV | 75 |
| COF_E_T2_4 | 63 KNLNSSQGVPDLLV | 76 |
| COV_E_T2_5 | 63 KNLNSSR-VPDLLV | 75 |

The amino acid differences of the designed sequences from the SARS2 reference sequence (SEQ ID NO: 41) are shown in the table below (with differences from the reference sequence highlighted in bold):

TABLE 10.13

| | SARS2 | | | | | |
|---|---|---|---|---|---|---|
| SARS2 E | Reference | | | | | |
| protein | Amino | COV_E_T2_1 | COV_E_T2_2 | COV_E_T2_3 | COV_E_T2_4 | COV_E_T2_5 |
| (SEQ ID | acid | Amino acid | Amino acid | Amino acid | Amino acid | Amino acid |
| NO: 41) | residue | residue | residue | residue | residue | residue |
| residue | (SEQ ID | (SEQ ID | (SEQ ID | (SEQ ID | (SEQ ID | (SEQ ID |
| position | NO: 41) | NO: 22) | NO: 23) | NO: 42) | NO: 43) | NO: 44) |
| 15 | N | N | N | A | A | A |
| 55 | S | T | T | S | T | T |
| 69 | R | Q | R | R | Q | R |
| 70 | — | G | — | — | G | — |
| Total no of | — | 3 | 1 | 1 | 4 | 2 |
| differences | | | | | | |
| from | | | | | | |
| reference | | | | | | |
| Percentage | — | 96 | 98.67 | 98.67 | 94.67 | 97.33 |
| identity with | | | | | | |
| reference | | | | | | |

According to the invention there is provided an isolated polypeptide, which comprises an amino acid sequence of SEQ ID NO:36.

According to the invention there is provided an isolated polypeptide, which comprises an amino acid sequence of SEQ ID NO:37.

According to the invention there is provided an isolated polypeptide, which comprises an amino acid sequence of SEQ ID NO:38.

According to the invention there is also provided an isolated polypeptide comprising an amino acid sequence of SEQ ID NO:42 (COV_E_T2_3), or an amino acid sequence which has at least 99% amino acid identity over its entire length with the amino acid sequence of SEQ ID NO: 42.

Optionally a polypeptide of the invention comprising an amino acid sequence of SEQ ID NO:42 (COV_E_T2_3), or an amino acid sequence which has at least 99% amino acid identity over its entire length with the amino acid sequence of SEQ ID NO:42, comprises amino acid residue A at a position corresponding to amino acid residue position 15 of SEQ ID NO:41.

According to the invention there is also provided an isolated polypeptide comprising an amino acid sequence of SEQ ID NO:42.

According to the invention there is also provided an isolated polypeptide comprising an amino acid sequence of SEQ ID NO:43 (COV_E_T2_4), or an amino acid sequence which has at least 95%, 96%, 97%, 98%, or 99% amino acid identity over its entire length with the amino acid sequence of SEQ ID NO:43.

Optionally a polypeptide of the invention comprising an amino acid sequence of SEQ ID NO:43 (COV_E_T2_4), or an amino acid sequence which has at least 95%, 96%, 97%, 98%, or 99% amino acid identity over its entire length with the amino acid sequence of SEQ ID NO:43, comprises at least one, or all of the following amino acid residues at positions corresponding to the amino acid residue positions of SEQ ID NO:41: 15A, 55T, 69Q, 70G.

According to the invention there is also provided an isolated polypeptide comprising an amino acid sequence of SEQ ID NO:43.

According to the invention there is also provided an isolated polypeptide comprising an amino acid sequence of SEQ ID NO:44 (COV_E_T2_5), or an amino acid sequence which has at least 98%, or 99% amino acid identity over its entire length with the amino acid sequence of SEQ ID NO:44.

Optionally a polypeptide of the invention comprising an amino acid sequence of SEQ ID NO:44 (COV_E_T2_5), or an amino acid sequence which has at least 98%, or 99% amino acid identity over its entire length with the amino acid sequence of SEQ ID NO:44, comprises at least one, or all of the following amino acid residues at positions corresponding to the amino acid residue positions of SEQ ID NO:41: 15A, 55T.

According to the invention there is also provided an isolated polypeptide comprising an amino acid sequence of SEQ ID NO:44.

According to the invention there is also provided an isolated polypeptide which comprises a coronavirus E protein with at least one of the following amino acid residues at positions corresponding to the amino acid residue positions of SEQ ID NO:41: 15A, 55T, 69Q, 70G.

Optionally an isolated polypeptide of the invention which comprises a coronavirus E protein, comprises the following amino acid residues at positions corresponding to the amino acid residue positions of SEQ ID NO:41: 15A, 55T.

Optionally an isolated polypeptide of the invention which comprises a coronavirus E protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:21.

### Designed Coronavirus Membrane (M) Protein Sequences

The applicant has also generated novel amino acid sequences for coronavirus Membrane (M) protein:
COV_M_T2_1 Sarbecovirus root ancestor (SEQ ID NO:24);
COV_M_T2_2 Epitope optimised version of SARS2 clade ancestor Node88b (D4 removed), SARS2 equivalent of B cell epitope from start and end added, and then T cell epitopes added whilst observing coevolving site constraints (SEQ ID NO:25).

The amino acid sequences of these designed sequences are:

>COV_M_T2_1/1-221 Sarbeco_M_root:
(SEQ ID NO: 24)
MADNGTITVE ELKQLLEQWN LVIGFLFLAW IMLLQFAYSN RNRFLYIIKL VFLWLLWPVT
LACFVLAAVY RINWVTGGIA IAMACIVGLM WLSYFVASFR LFARTRSMWS FNPETNILLN
VPLRGTILTR PLMESELVIG AVIIRGHLRM AGHSLGRCDI KDLPKEITVA TSRTLSYYKL
GASQRVGTDS GFAAYNRYRI GNYKLNTDHA GSNDNIALLV Q
>COV_M_T2_2/1-222 Sarbeco_M_Node88b_epitope_optimised:
(SEQ ID NO: 25)
MADSNGTITV EELKKLLEQW NLVIGFLFLT WICLLQFAYS NRNRFLYIIK LIFLWLLWPV
TLACFVLAAV YRINWVIGGI AIAMACIVGL MWLSYFVASF RLFARTRSMW SFNPETNILL
NVPLRGSIIT RPLMESELVI GAVILRGHLR MAGHSLGRCD IKDLPKEITV ATSRTLSYYK
LGASQRVASD SGFAVYNRYR IGNYKLNTDH SSSSDNIALL VQ

As described in Example 11 below, FIG. 8 shows alignment of a SARS2 reference M protein sequence (SEQ ID NO:26) with the designed sequences. The alignment shown in FIG. 8 highlights the amino acid differences between the SARS2 reference M protein sequence and the COV_M_T2_1 and COV_M_T2_2 designed sequences, as shown in the table below:

TABLE 11.1

| SARS2 M | SARS2 | COV_M_T2_1 | COV_M_T2_2 |
|---|---|---|---|
| reference | Reference | Amino | Amino |
| protein | Amino acid | acid | acid |
| residue | residue | residue | residue |
| position | (SQ ID | (SEQ ID | (SEQ ID |
| (SEQ ID NO: 26) | NO: 26) | NO: 24) | NO: 25) |
| 4 | S | — | S |
| 15 | K | Q | K |
| 30 | T | A | T |
| 33 | C | M | C |
| 40 | A | S | S |
| 52 | I | V | I |
| 76 | I | V | V |
| 87 | L | I | I |
| 97 | I | V | V |
| 125 | H | R | R |
| 127 | T | T | S |
| 134 | L | M | M |
| 145 | L | I | L |
| 151 | I | M | M |
| 155 | H | S | S |
| 188 | A | G | A |
| 189 | G | T | S |
| 195 | A | A | V |
| 197 | S | N | N |
| 211 | S | A | S |
| 212 | S | G | S |
| 214 | S | N | S |

According to the invention there is also provided an isolated polypeptide which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24.

Optionally an isolated polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, comprises at least one of the amino acid residues, at a position corresponding to the amino acid residue position of SEQ ID NO:26, as shown in the table below:

TABLE 11.2

| SARS2 M protein | COV_M_T2_1 Amino |
|---|---|
| residue position | acid residue |
| 40 | S |
| 76 | V |
| 87 | I |
| 97 | V |
| 125 | R |
| 134 | M |
| 151 | M |
| 155 | S |
| 197 | N |

Optionally an isolated polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, comprises at least five of the amino acid residues, at positions corresponding to the amino acid residue positions of SEQ ID NO:26, as shown in Table 11.2.

Optionally an isolated polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, comprises all of the amino acid residues, at positions corresponding to the amino acid residue positions of SEQ ID NO:26, as shown in Table 11.2.

Optionally an isolated polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, comprises at least one of the amino acid residues, at a position corresponding to the amino acid residue position of SEQ ID NO:26, as shown in the table below:

TABLE 11.3

| SARS2 M protein | COV_M_T2_1 Amino |
|---|---|
| residue position | acid residue |
| 4 | — (deletion) |
| 15 | Q |
| 30 | A |
| 33 | M |
| 40 | S |
| 52 | V |
| 76 | V |
| 87 | I |
| 97 | V |
| 125 | R |
| 134 | M |
| 145 | I |
| 151 | M |
| 155 | S |
| 188 | G |
| 189 | T |
| 197 | N |
| 211 | A |
| 212 | G |
| 214 | N |

Optionally an isolated polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, comprises at least five of the amino acid residues, at positions corresponding to the amino acid residue positions of SEQ ID NO:26, as shown in Table 11.3.

Optionally an isolated polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, comprises at least ten of the amino acid residues, at positions corresponding to the amino acid residue positions of SEQ ID NO:26, as shown in Table 11.3.

Optionally an isolated polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, comprises at least fifteen of the amino acid residues, at positions corresponding to the amino acid residue positions of SEQ ID NO:26, as shown in Table 11.3.

Optionally an isolated polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, comprises all of the amino acid residues, at positions corresponding to the amino acid residue positions of SEQ ID NO:26, as shown in Table 11.3.

According to the invention there is also provided an isolated polypeptide comprising an amino acid sequence of SEQ ID NO:24.

There is also provided according to the invention an isolated polypeptide which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally an isolated polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25, comprises at least one of the amino acid residues, at a position corresponding to the amino acid residue positions of SEQ ID NO:25, as shown in the table below:

TABLE 11.4

| SARS2 M protein | COV_M_T2_2 Amino |
|---|---|
| residue position | acid residue |
| 40 | S |
| 76 | V |
| 87 | I |
| 97 | V |
| 125 | R |
| 134 | M |
| 151 | M |
| 155 | S |
| 197 | N |

Optionally an isolated polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25, comprises at least five of the amino acid residues, at positions corresponding to the amino acid residue positions of SEQ ID NO:25, as shown in Table 11.4.

Optionally an isolated polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25, comprises all of the amino acid residues, at positions corresponding to the amino acid residue positions of SEQ ID NO:25, as shown in Table 11.4.

Optionally an isolated polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25, comprises at least one of the amino acid residues, at a position corresponding to the amino acid residue position of SEQ ID NO:25, as shown in the table below:

TABLE 11.5

| SARS2 M protein | COV_M_T2_2 Amino |
|---|---|
| residue position | acid residue |
| 40 | S |
| 76 | V |
| 87 | I |
| 97 | V |
| 125 | R |
| 127 | S |
| 134 | M |
| 151 | M |
| 155 | S |
| 189 | S |
| 195 | V |
| 197 | N |

Optionally an isolated polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25, comprises at least five of the amino acid residues, at positions corresponding to the amino acid residue positions of SEQ ID NO:25, as shown in Table 11.5.

Optionally an isolated polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25, comprises at least ten of the amino acid residues, at positions corresponding to the amino acid residue positions of SEQ ID NO:25, as shown in Table 11.5.

Optionally an isolated polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25, comprises all of the amino acid residues, at positions corresponding to the amino acid residue positions of SEQ ID NO:25, as shown in Table 11.5.

According to the invention there is also provided an isolated polypeptide comprising an amino acid sequence of SEQ ID NO:25.

According to the invention there is also provided an isolated polypeptide, which comprises a coronavirus M protein with any, or all of the amino acid residues at positions corresponding to the amino acid residue positions as shown in the table below:

TABLE 11.6

| M protein residue | Amino acid |
|---|---|
| position | residue |
| 40 | S |
| 76 | V |
| 87 | I |
| 97 | V |
| 125 | R |
| 134 | M |
| 151 | M |
| 155 | S |
| 197 | N |

According to the invention there is also provided an isolated polypeptide, which comprises a coronavirus M protein with any, or all of the amino acid residues at positions corresponding to the amino acid residue positions as shown in the table below:

TABLE 11.7

| M protein residue | Amino acid |
|---|---|
| position | residue |
| 4 | — (deletion) |
| 15 | Q |
| 30 | A |
| 33 | M |
| 40 | S |
| 52 | V |
| 76 | V |
| 87 | I |
| 97 | V |
| 125 | R |
| 134 | M |
| 145 | I |
| 151 | M |
| 155 | S |
| 188 | G |
| 189 | T |
| 197 | N |
| 211 | A |
| 212 | G |
| 214 | N |

According to the invention there is also provided an isolated polypeptide, which comprises a coronavirus M protein with any, or all of the amino acid residues at positions corresponding to the amino acid residue positions as shown in the table below:

TABLE 11.8

| M protein residue | Amino acid |
|---|---|
| position | residue |
| 40 | S |
| 76 | V |
| 87 | I |
| 97 | V |
| 125 | R |
| 134 | M |
| 151 | M |
| 155 | S |
| 197 | N |

According to the invention there is also provided an isolated polypeptide, which comprises a coronavirus M protein with any, or all of the amino acid residues at positions corresponding to the amino acid residue positions as shown in the table below:

TABLE 11.9

| M protein residue | Amino acid |
|---|---|
| position | residue |
| 40 | S |
| 76 | V |
| 87 | I |
| 97 | V |
| 125 | R |
| 127 | S |
| 134 | M |
| 151 | M |
| 155 | S |
| 189 | S |
| 195 | V |
| 197 | N |

Optionally an isolated polypeptide of the invention which comprises a coronavirus M protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:26.

We have made further new M protein designs (COV_M_T2_3, COV_M_T2_4, COV_M_T2_5)). In these designs, we have deleted the first and the second transmembrane region of the membrane protein to abrogate its interaction with the S protein:
The string construct with S, M and E was showing higher order aggregates.
Abrogation of interaction between S and M—can reduce aggregation.
M-del constructs (Cov_M_T2_(3-5)) designed to abrogate the interaction with S.

FIG. 20 shows an illustration of the M protein. Interaction between the M, E and N proteins is important for viral assembly. The M protein also binds to the nucleocapsid, and this interaction promotes the completion of virion assembly. These interactions have been mapped to the C-terminus of the endo-domain of the M protein, and the C-terminal domain of the N-protein. In FIG. 20, * denotes identification of immunodominant epitopes on the membrane protein of the Severe Acute Respiratory Syndrome-Associated Coronavirus, and ** denotes mapping of the Coronavirus membrane protein domains involved in interaction with the Spike protein.

The amino acid sequences of the new M protein designs are given below:

>COV_M_T2_3
(SEQ ID NO: 48)
MADSNGTITV EELKKLLEQI TGGIAIAMAC LVGLMWLSYF IASFRLFART RSMWSFNPET NILLNVPLHG
TILTRPLLES ELVIGAVILR GHLRIAGHHL GRCDIKDLPK EITVATSRTL SYYKLGASQR VAGDSGFAAY
SRYRIGNGKL NTDHSSSSDN IALLVQ
>COV_M_T2_4
(SEQ ID NO: 49)
MADNGTITVE ELKQLLEQVT GGIAIAMACI VGLMWLSYFV ASFRLFARTR SMWSFNPETN ILLNVPLRGT
ILTRPLMESE LVIGAVIIRG HLRMAGHSLG RCDIKDLPKE ITVATSRTLS YYKLGASQRV GTDSGFAAYN
RYRIGNGKLN TDHAGSNDNI ALLVQ
>COV_M_T2_5
(SEQ ID NO: 50)
MADSNGTITV EELKKLLEQV TGGIAIAMAC IVGLMWLSYF VASFRLFART RSMWSFNPET NILLNVPLRG
SIITRPLMES ELVIGAVILR GHLRMAGHSL GRCDIKDLPK EITVATSRTL SYYKLGASQR VASDSGFAVY
NRYRIGNGKL NTDHSSSSDN IALLVQ

Sequence alignment of the new M protein designs (COV_M_T2_3, COV_M_T2_4, COV_M_T2_5) with the previous M protein designs (COV_M_T1_1, COV_M_T2_1, COV_M_T2_2) is shown below.

| | | |
|---|---|---|
| COV_M_T1_1 | MADSNGTITVEELKKLLEQWNLVIGFLFLTWICLLQFAYANRNRFLYIIKLIFLWLLWPVTLA | 63 |
| COV_M_T2_3 | MADSNGTITVEELKKLLEQ-------------------------------------------- | 19 |
| COV_M_T2_1 | MAD-NGTITVEELKQLLEQWNLVIGFLFLAWIMLLQFAYSNRNRFLYIIKLVFLWLLWPVTLA | 62 |
| COV_M_T2_4 | MAD-NGTITVEELKQLLEQ-------------------------------------------- | 18 |
| COV_M T2_2 | MADSNGTITVEELKLLLEQWNLVIGFLFLTWICLLQFAYSNRNRFLYIIKLIFLWLLWPVTLA | 63 |
| COV_M_72_5 | MADSNGTITVEELKKLLEQ-------------------------------------------- | 19 |
| COV_M_T1_1 | CFVLAAVYRINWITGGIAIAMACLVGLMWLSYFIASFRLFARTRSMWSFNPETNILLNVPLHG | 126 |
| COV_M_T2_3 | ------------ITGGIAIAMACLVGLMWLSYFIASFRLFARTRSMWSFNPETNILLNVPLHG | 70 |
| COV_M_T2_1 | CFVLAAVYRINWVTGGIAIAMACIVGLMWLSYFVASFRLFARTRSMWSFNPETNILLNVPLRG | 125 |
| COV_M_T2_4 | ------------VTGGIAIAMACIVGLMWLSYFVASFRLFARTRSMWSFNPETNILLNVPLRG | 69 |
| COV_M T2_2 | CFVLAAVYRINWVTGGIAIAMACIVGLMWLSYFVASFRLFARTRSMWSFNPETNILLNVPLRG | 126 |
| COV_M_72_5 | ------------VTGGIAIAMACIVGLMWLSYFVASFRLFARTRSMWSFNPETNILLNVPLRG | 70 |
| COV_M_T1_1 | TILTRPLLESELVIGAVILRGHLRIAGHHLGRCDIKDLPKEITVATSRTLSYYKLGASQRVAG | 189 |
| COV_M_T2_3 | TILTRPLLESELVIGAVILRGHLRIAGHHLGRCDIKDLPKEITVATSRTLSYYKLGASQRVAG | 133 |
| COV_M_T2_1 | TILTRPLMESELVIGAVIIRGHLAMAGHSLGRCDIKDLPKEITVATSRTLSYYKLGASQRVGT | 188 |
| COV_M_T2_4 | TILTRPLMESELVIGAVIIRGHLRMAGHSLGRCDIKDLPKEITVATSRTLSYYKLGASQRVGT | 132 |
| COV_M T2_2 | SIITRPLMESELVIGAVILRGHLRMAGHSLGRCDIKDLPKEITVATSRTLSYYKLGASQRVAS | 189 |
| COV_M_72_5 | SIITRPLMESELVIGAVILRGHLRMAGHSLGRCDIKDLPKEITVATSRTLSYYKLGASQRVAS | 133 |
| COV_M_T1_1 | DSGFAAYSRYRIGNYKLNTDHSSSSDNIALLVQ | 222 |
| COV_M_T2_3 | DSGFAAYSRYRIGNGKLNTDHSSSSDNIALLVQ | 166 |
| COV_M_T2_1 | DSGFAAYNRYRIGNYKLNTDHAGSNDNIALLVQ | 221 |
| COV_M_T2_4 | DSGFAAYNRYRIGNGKLNTDHAGSNDNIALLVQ | 165 |
| COV_M T2_2 | DSGFAVYNRYRIGNYKLNTDHSSSSDNIALLVQ | 222 |
| COV_M_72_5 | DSGFAVYNRYRIGNGKLNTDHSSSSDNIALLVQ | 166 |

The amino acid differences of the designed sequences from the SARS2 M protein reference sequence are shown in the table below (with differences from the reference sequence highlighted in bold):

TABLE 11.10

| | SARS2 Reference | COV_M_T2_1 | COV_M_T2_2 | COV_M_T2_3 | COV_M_T2_4 | COV_M_T2_5 |
|---|---|---|---|---|---|---|
| SARS2 M protein | Amino acid residue | Amino acid | Amino acid | Amino acid | Amino acid | Amino acid |
| residue position | (COV_M_T1_1) (SEQ | residue (SEQ | residue (SEQ | residue (SEQ | residue (SEQ | residue (SEQ |
| (SEQ ID NO: 26) | ID NO: 26) | ID NO: 24) | ID NO: 25) | ID NO: 48) | ID NO: 49) | ID NO: 50) |
| 4 | S | Deleted | S | S | Deleted | S |
| 15 | K | Q | K | K | Q | K |
| 20-75 | | | | Deleted | Deleted | Deleted |
| 30 | T | A | T | | | |
| 33 | C | M | C | | | |
| 40 | A | S | S | | | |
| 52 | I | V | I | | | |
| 76 | I | V | V | I | V | V |
| 87 | L | I | I | L | I | I |
| 97 | I | V | V | I | V | V |
| 125 | H | R | R | H | R | R |
| 127 | T | T | S | T | T | S |
| 129 | L | L | I | L | L | I |
| 134 | L | M | M | L | M | M |
| 145 | L | I | L | L | I | L |
| 151 | I | M | M | I | M | M |
| 155 | H | S | S | H | S | S |
| 188 | A | G | A | A | G | A |
| 189 | G | T | S | G | T | S |
| 195 | A | A | V | A | A | V |
| 197 | S | N | N | S | N | N |
| 204 | Y | Y | Y | G | G | G |
| 21 | S | A | S | S | A | S |
| 212 | S | G | S | S | G | S |
| 214 | S | N | S | S | N | S |
| Total no of | — | 20 | 13 | 57 | 73 | 69 |
| differences from | | | | | | |
| reference | | | | | | |
| Percentage identity | — | 90.99% | 94.14% | 74.32% | 67.12% | 68.92% |
| with reference | | | | | | |

According to the invention there is also provided an isolated polypeptide which comprises an amino acid sequence of SEQ ID NO:48, or an amino acid sequence which has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:48.

Optionally an isolated polypeptide of the invention which comprises an amino acid sequence of SEQ ID NO:48, or an amino acid sequence which has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:48, comprises a deletion of amino acid residues at positions corresponding to positions 20-75 of SEQ ID NO:26.

Optionally an isolated polypeptide of the invention which comprises an amino acid sequence of SEQ ID NO:48, or an amino acid sequence which has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:48, comprises amino acid residue G at a position corresponding to amino acid residue position 204 of SEQ ID NO:26.

According to the invention there is also provided an isolated polypeptide comprising an amino acid sequence of SEQ ID NO:48.

According to the invention there is also provided an isolated polypeptide which comprises an amino acid sequence of SEQ ID NO:49, or an amino acid sequence which has at least 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:49.

Optionally an isolated polypeptide of the invention which comprises an amino acid sequence of SEQ ID NO:49, or an amino acid sequence which has at least 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:49, comprises a deletion of amino acid residues at positions corresponding to positions 20-75 of SEQ ID NO:26.

Optionally an isolated polypeptide of the invention which comprises an amino acid sequence of SEQ ID NO:49, or an amino acid sequence which has at least 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:49, comprises at least one, or all, of the amino acid residues, at a position corresponding to the amino acid residue position of SEQ ID NO:26, as shown in the table below:

TABLE 11.11

| SARS2 M protein | |
|---|---|
| residue position | Amino acid |
| (SEQ ID NO: 26) | residue |
| 20-75 | Deleted |
| 76 | V |
| 87 | I |
| 97 | V |
| 125 | R |
| 134 | M |
| 151 | M |
| 155 | S |
| 189 | T |
| 197 | N |
| 204 | G |

Optionally an isolated polypeptide of the invention which comprises an amino acid sequence of SEQ ID NO:49, or an amino acid sequence which has at least 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:49, comprises at least one, or all, of the amino acid residues, at a position corresponding to the amino acid residue position of SEQ ID NO:26, as shown in the table below:

TABLE 11.12

| | COV_M_T2_4 |
|---|---|
| SARS2 M protein | Amino acid |
| residue position | residue (SEQ |
| (SEQ ID NO: 26) | ID NO: 49) |
| 4 | Deleted |
| 15 | Q |
| 20-75 | Deleted |
| 76 | V |
| 87 | I |
| 97 | V |
| 125 | R |
| 134 | M |
| 145 | I |
| 151 | M |
| 155 | S |
| 188 | G |
| 189 | T |
| 197 | N |
| 204 | G |
| 211 | A |
| 212 | G |
| 214 | N |

According to the invention there is also provided an isolated polypeptide comprising an amino acid sequence of SEQ ID NO:49.

According to the invention there is also provided an isolated polypeptide which comprises an amino acid sequence of SEQ ID NO:50, or an amino acid sequence which has at least 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:50.

Optionally an isolated polypeptide of the invention which comprises an amino acid sequence of SEQ ID NO:50, or an amino acid sequence which has at least 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:50, comprises a deletion of amino acid residues at positions corresponding to positions 20-75 of SEQ ID NO:26.

Optionally an isolated polypeptide of the invention which comprises an amino acid sequence of SEQ ID NO:50, or an amino acid sequence which has at least 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:50, comprises at least one, or all, of the amino acid residues, at a position corresponding to the amino acid residue position of SEQ ID NO: 26, as shown in the table below:

TABLE 11.11

| SARS2 M protein | |
|---|---|
| residue position | Amino acid |
| (SEQ ID NO: 26) | residue |
| 20-75 | Deleted |
| 76 | V |
| 87 | I |
| 97 | V |
| 125 | R |
| 134 | M |
| 151 | M |
| 155 | S |
| 189 | T |
| 197 | N |
| 204 | G |

Optionally an isolated polypeptide of the invention which comprises an amino acid sequence of SEQ ID NO:50, or an amino acid sequence which has at least 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:50, comprises at least one, or all, of the amino acid residues, at a position corresponding to the amino acid residue position of SEQ ID NO: 26, as shown in the table below:

TABLE 11.13

| | COV_M_T2_5 |
|---|---|
| SARS2 M protein | Amino acid |
| residue position | residue (SEQ |
| (SEQ ID NO: 26) | ID NO: 50) |
| 20-75 | Deleted |
| 76 | V |
| 87 | I |
| 97 | V |
| 125 | R |
| 127 | S |
| 129 | I |
| 134 | M |
| 151 | M |
| 155 | S |
| 189 | S |
| 195 | V |
| 197 | N |
| 204 | G |

According to the invention there is also provided an isolated polypeptide comprising an amino acid sequence of SEQ ID NO:50.

According to the invention there is also provided an isolated polypeptide, which comprises a coronavirus M protein with any, or all of the amino acid residues at positions corresponding to the amino acid residue positions as shown in the table below:

TABLE 11.11

| SARS2 M protein | |
|---|---|
| residue position | Amino acid |
| (SEQ ID NO: 26) | residue |
| 20-75 | Deleted |
| 76 | V |
| 87 | I |
| 97 | V |
| 125 | R |
| 134 | M |
| 151 | M |
| 155 | S |
| 189 | T |
| 197 | N |
| 204 | G |

According to the invention there is also provided an isolated polypeptide, which comprises a coronavirus M protein with any, or all of the amino acid residues at positions corresponding to the amino acid residue positions as shown in the table below:

TABLE 11.12

| | COV_M_T2_4 |
|---|---|
| SARS2 M protein | Amino acid |
| residue position | residue (SEQ |
| (SEQ ID NO: 26) | ID NO: 49) |
| 4 | Deleted |
| 15 | Q |
| 20-75 | Deleted |
| 76 | V |
| 87 | I |
| 97 | V |
| 125 | R |
| 134 | M |
| 145 | I |
| 151 | M |
| 155 | S |
| 188 | G |
| 189 | T |
| 197 | N |
| 204 | G |
| 211 | A |
| 212 | G |
| 214 | N |

According to the invention there is also provided an isolated polypeptide, which comprises a coronavirus M protein with any, or all of the amino acid residues at positions corresponding to the amino acid residue positions as shown in the table below:

TABLE 11.13

| | COV_M_T2_5 |
|---|---|
| SARS2 M protein | Amino acid |
| residue position | residue (SEQ |
| (SEQ ID NO: 26) | ID NO: 50) |
| 20-75 | Deleted |
| 76 | V |
| 87 | I |
| 97 | V |
| 125 | R |
| 127 | S |
| 129 | I |
| 134 | M |
| 151 | M |
| 155 | S |
| 189 | S |
| 195 | V |
| 197 | N |
| 204 | G |

Optionally an isolated polypeptide of the invention which comprises a coronavirus M protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:26.

### Designed Coronavirus Nucleoprotein (N) Sequences

We have made new N protein designs, COV_N_T2_1 (SEQ ID NO:46) and COV_N_T2_2 (SEQ ID NO: 47). The amino acid sequences of these designs is shown below, and in Example 15. Sequence COV_N_T2_2 was designed using a methodology and algorithm which selected predicted epitopes to include based on their conservation across the sarbecoviruses (whilst minimising redundancy), the frequency and number of MHC alleles the epitope is restricted by the predicted epitope quality, and a handful of user specified weightings.

>YP_009724397.2/1-419 nucleocapsid phosphoprotein [SARS-CoV-2] (reference sequence)
(SEQ ID NO: 45)
MSDNGPQ-NQ RNAPRITFGG PSDSTGSNQN GERSGARSKQ RRPQGLPNNT ASWFTALTQH GKEDLKFPRG
QGVPINTNSS PDDQIGYYRR ATRRIRGGDG KMKDLSPRWY FYYLGTGPEA GLPYGANKDG IIWVATEGAL
NTPKDHIGTR NPANNAAIVL QLPQGTTLPK GFYAEGSRGG SQASSRSSSR SRNSSRNSTP GSSRGTSPAR
MAGNGGDAAL ALLLLDRLNQ LESKMSGKGQ QQQGQTVTKK SAAEASKKPR QKRTATKAYN VTQAFGRRGP
EQTQGNFGDQ ELIRQGTDYK HWPQIAQFAP SASAFFGMSR IGMEVTPSGT WLTYTGAIKL DDKDPNFKDQ
VILLNKHIDA YKTFPPTEPK KDKKKKADET QALPQRQKKQ QTVTLLPAAD LDDFSKQLQQ SMSSA--DST
QA
>COV_N_T2_1/1-418 Node1b 321-323 deleted
(SEQ ID NO: 46)
MSDNGPQ-NQ RSAPRITFGG PSDSTDNNQN GERSGARPKQ RRPQGLPNNT ASWFTALTQH GKEDLRFPRG
QGVPINTNSG KDDQIGYYRR ATRRVRGGDG KMKELSPRWY FYYLGTGPEA ALPYGANKEG IVWVATEGAL
NTPKDHIGTR NPNNNAAIVL QLPQGTTLPK GFYAEGSRGG SQASSRSSSR SRGNSRNSTP GSSRGTSPAR
MASGGGDTAL ALLLLDRLNQ LESKVSGKGQ QQQGQTVTKK SAAEASKKPR QKRTATKQYN VTQAFGRRGP
EQTQGNFGDQ ELIRQGTDYK HWPQIAQFAP SASAFFGMSR ---EVTPSGT WLTYHGAIKL DDKDPQFKDN
VILLNKHIDA YKTFPPTEPK KDKKKKADEA QPLPQRQKKQ PTVTLLPAAD LDDFSKQLQN SMSGASADST
QA
>COV_N_T2 2/1-417 epitope optimised 321-323 deleted
(SEQ ID NO: 47)
MTDNGQQ-GP RNAPRITF-G VSDNFDNNQD GGRSGARPKQ RRPQGLPNNT ASWFTALTQH GKEDLRFPRG
QGVPINTNSS PDDQIGYYRR ATRRIRGGDG KMKDLSPRWY FYYLGTGPEA ALPYGANKEG IVWVATEGAL
NTPKDHIGTR NPNNNAAIVL QLPQGTTLPK GFYAEGSRGG SQASSRSSSR SRNSSRNSTP GSSRGTSPAR
NLQAGGDTAL ALLLLDRINQ LESKMSGKGQ QQQGQTVTKK SAAEASKKPR QKRTATKQYN VTQAFGRRGP
EQTQGNFGDQ ELIRQGTDYK QWPQIAQFAP SASAFFGMSR ---EVTPSGT WLTYTGAIKL DDKDPQFKDN
VILLNKHIDA YKTFPPTEPK KDKKKKADEA QPLPQRQKKQ QTVTLLPAAD LDDFSRQLQN SMSGASADST
QA

Alignment of the N protein designs with SARS2 N protein reference sequence is shown below.

| | | |
|---|---|---|
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |

[symbol]

indicates data missing or illegible when filed

The amino acid differences of the designed sequences from the SARS2 reference sequence are shown in the Table 12.1 below (with differences from the reference sequence highlighted in bold, and differences that are common to all the designed sequences underlined):

TABLE 12.1

| SARS2 N | | | |
|---|---|---|---|
| protein | | | |
| reference | | | |
| (SEQ ID | | | N_T2_2 |
| NO: 45) | | N_T2_1 amino | amino acid |
| residue | SARS2 Reference | acid residue | residue (SEQ |
| position | amino acid residue | (SEQ ID NO: 46) | ID NO: 47) |
| 2 | S | S | T |
| 6 | P | P | Q |
| 8 | N | N | G |
| 9 | Q | Q | P |
| 11 | N | S | N |
| 18 | G | G | — |
| 20 | P | P | V |
| 23 | S | S | N |
| 24 | T | T | F |
| 25 | G | D | D |
| 26 | S | N | N |
| 29 | N | N | D |
| 31 | E | E | G |
| 37 | S | P | P |
| 65 | K | R | R |
| 79 | S | G | S |
| 80 | P | K | P |
| 94 | I | V | I |
| 103 | D | E | D |
| 120 | G | A | A |
| 128 | D | E | E |
| 131 | I | V | V |
| 152 | A | N | N |
| 192 | N | G | N |
| 193 | S | N | S |
| 211 | A | A | L |
| 212 | G | S | Q |
| 213 | N | G | A |
| 217 | A | T | T |
| 234 | M | V | M |
| 267 | A | Q | Q |
| 300 | H | H | Q |
| 320 | I | — | — |
| 321 | G | — | — |
| 322 | M | — | — |
| 334 | T | H | T |
| 345 | N | Q | Q |
| 349 | Q | N | N |
| 379 | T | A | A |
| 390 | Q | P | Q |
| 406 | K | K | R |
| 409 | Q | N | N |
| 413 | S | G | G |
| 415 | — | S | S |
| 416 | — | A | A |
| Total no of | — | 31 | 35 |
| differences | | | |
| from | | | |
| reference | | | |
| Percentage | — | 92.60 | 91.65 |
| identity with | | | |
| reference | | | |

Positions 415 and 416 of the SARS2 N protein reference residue position column are italicised as they are not residues of the reference sequences, but include insertions in the N_T2_1 and N_T2_2 sequences.

The amino acid changes common to both of the designed sequences are summarised in the table below:

TABLE 12.2

| SARS2 N | Amino acid |
|---|---|
| protein | residue of |
| (SEQ ID | designed |
| NO: 45) | sequences |
| residue | (SEQ ID |
| position | Nos: 46, 47) |
| 26 | N |
| 37 | P |
| 65 | R |
| 120 | A |
| 128 | E |
| 131 | V |
| 152 | N |
| 217 | T |
| 267 | Q |
| 345 | Q |
| 349 | N |
| 379 | A |
| 409 | N |
| 413 | G |
| 415 | S (insertion) |
| 416 | A (insertion) |

Optional additional changes are summarised in the table below:

TABLE 12.3

| SARS2 N | |
|---|---|
| protein | Amino acid |
| (SEQ ID | residue of |
| NO: 45) | designed |
| residue | sequence (SEQ |
| position | ID NO: 46) |
| 11 | S |
| 79 | G |
| 80 | K |
| 94 | V |
| 103 | E |
| 192 | G |
| 193 | N |
| 212 | S |
| 213 | G |
| 234 | V |
| 320 | — |
| 321 | — |
| 322 | — |
| 334 | H |
| 390 | P |

Alternative optional additional changes are summarised in the table below:

TABLE 12.4

| SARS2 N | |
|---|---|
| protein | |
| (SEQ ID | |
| NO: 45) | Amino acid |
| residue | residue (SEQ |
| position | ID NO: 47) |
| 2 | T |
| 6 | Q |
| 8 | G |
| 9 | P |
| 18 | — |
| 20 | V |
| 23 | N |
| 24 | F |
| 25 | D |
| 29 | D |
| 31 | G |
| 211 | L |
| 212 | Q |
| 213 | A |
| 300 | Q |
| 320 | — |
| 321 | — |
| 322 | — |
| 406 | R |

According to the invention there is provided an isolated polypeptide which comprises an amino acid sequence of SEQ ID NO:46 (COV_N_T2_1), or an amino acid sequence which has at least 93%, 94%, 95%, 96%, 97%, 98%, or 99% amino acid identity over its entire length with the amino acid sequence of SEQ ID NO:46.

Optionally a polypeptide of the invention comprising an isolated polypeptide comprising an amino acid sequence of SEQ ID NO:46, or an amino acid sequence which has at least 93%, 94%, 95%, 96%, 97%, 98%, or 99% amino acid identity over its entire length with the amino acid sequence of SEQ ID NO:46, further comprises at least one, or all of the amino acid residues at positions corresponding to the amino acid residue positions as shown in Table 12.2 above.

Optionally a polypeptide of the invention comprising an isolated polypeptide comprising an amino acid sequence of SEQ ID NO:46, or an amino acid sequence which has at least 93%, 94%, 95%, 96%, 97%, 98%, or 99% amino acid identity over its entire length with the amino acid sequence of SEQ ID NO:46, further comprises at least one, or all of the amino acid residues at positions corresponding to the amino acid residue positions as shown in Table 12.3 above.

According to the invention there is provided an isolated polypeptide which comprises an amino acid sequence of SEQ ID NO:46 (COV_N_T2_1).

According to the invention there is also provided an isolated polypeptide which comprises an amino acid sequence of SEQ ID NO:47 (COV_N_T2_2), or an amino acid sequence which has at least 92%, 93%, 94%, 95%, 96%, 97%, 98%, or 99% amino acid identity over its entire length with the amino acid sequence of SEQ ID NO:47.

Optionally a polypeptide of the invention comprising an isolated polypeptide comprising an amino acid sequence of SEQ ID NO:47, or an amino acid sequence which has at least 92%, 93%, 94%, 95%, 96%, 97%, 98%, or 99% amino acid identity over its entire length with the amino acid sequence of SEQ ID NO:47, further comprises at least one, or all of the amino acid residues at positions corresponding to the amino acid residue positions as shown in Table 12.2 above.

Optionally a polypeptide of the invention comprising an isolated polypeptide comprising an amino acid sequence of SEQ ID NO:47, or an amino acid sequence which has at least 92%, 93%, 94%, 95%, 96%, 97%, 98%, or 99% amino acid identity over its entire length with the amino acid sequence of SEQ ID NO:47, further comprises at least one, or all of the amino acid residues at positions corresponding to the amino acid residue positions as shown in Table 12.4 above.

According to the invention there is also provided an isolated polypeptide which comprises an amino acid sequence of SEQ ID NO:47 (COV_N_T2_2).

According to the invention there is also provided an isolated polypeptide, which comprises a coronavirus N protein with at least one, or all of the amino acid residues at positions corresponding to the amino acid residue positions of SEQ ID NO:45 as shown in Table 12.2 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least five amino acid residues at positions corresponding to the amino acid residue positions of SEQ ID NO: 45, as shown in Table 12.2 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least ten amino acid residues at positions corresponding to the amino acid residue positions of SEQ ID NO: 45, as shown in Table 12.2 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least fifteen amino acid residues at positions corresponding to the amino acid residue positions of SEQ ID NO: 45, as shown in Table 12.2 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least one, or all of the amino acid residues at positions corresponding to the amino acid residue positions of SEQ ID NO:45, as shown in Table 12.3 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least five of the amino acid residues at positions corresponding to the amino acid residue positions of SEQ ID NO: 45, as shown in Table 12.3 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least ten of the amino acid residues at positions corresponding to the amino acid residue positions of SEQ ID NO: 45, as shown in Table 12.3 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least one, or all of the amino acid residues at positions corresponding to the amino acid residue positions of SEQ ID NO:45, as shown in Table 12.4 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least five of the amino acid residues at positions corresponding to the amino acid residue positions of SEQ ID NO: 45, as shown in Table 12.4 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least ten of the amino acid residues at positions corresponding to the amino acid residue positions of SEQ ID NO: 45, as shown in Table 12.4 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least fifteen of the amino acid residues at positions corresponding to the amino acid residue positions of SEQ ID NO:45, as shown in Table 12.4 above.

Optionally an isolated polypeptide of the invention which comprises a coronavirus N protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:45.

Polypeptides of the invention are particularly advantageous because they can elicit a broadly neutralising immune response to several different types of coronavirus, in particular several different types of β-coronavirus. Polypeptides of the invention comprising an amino acid sequence of SEQ ID NO:15 (or an amino acid sequence which has at least 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 15), or SEQ ID NO:17 (or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17) are also advantageous because they lack non-neutralising epitopes that may result in virus immune evasion and disease progression by ADE (or ADE-like pro-inflammatory responses).

Similarly, polypeptides of the invention comprising a novel designed coronavirus E protein amino acid sequence (for example, an amino acid sequence of SEQ ID NO:22, or an amino acid sequence which has at least 95%, 96%, 97%, 98%, or 99% amino acid identity over its entire length with the amino acid sequence of SEQ ID NO:22, or an amino acid sequence of SEQ ID NO:23, or an amino acid sequence which has at least 98%, or 99% amino acid identity over its entire length with the amino acid sequence of SEQ ID NO:23), or a coronavirus M protein amino acid sequence (for example, an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or an amino acid sequence of SEQ ID NO:25, or an amino acid sequence which has at least 95%, 96%, 97%, 98%, or 99% amino acid identity over its entire length with the amino acid sequence of SEQ ID NO:25) are advantageous because they lack non-neutralising epitopes that may result in virus immune evasion and disease progression by ADE (or ADE-like pro-inflammatory responses).

A polypeptide of the invention may include one or more conservative amino acid substitutions. Conservative amino acid substitutions are those substitutions that, when made, least interfere with the properties of the original polypeptide, that is, the structure and especially the function of the protein is conserved and not significantly changed by such substitutions. Examples of conservative substitutions are shown below:

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, serine or threonine, is substituted for (or by) a hydrophobic residue, for example, leucine, isoleucine, phenylalanine, valine or alanine; (b) a cysteine or proline is substituted for (or by) any other residue; (c) a residue having an electropositive side chain, for example, lysine, arginine, or histidine, is substituted for (or by) an electronegative residue, for example, glutamate or aspartate; or (d) a residue having a bulky side chain, for example, phenylalanine, is substituted for (or by) one not having a side chain, for example, glycine.

The term “broadly neutralising immune response” is used herein to mean an immune response elicited in a subject that is sufficient to inhibit (i.e. reduce), neutralise or prevent infection, and/or progress of infection, of a virus within the coronavirus family. Optionally a broadly neutralising immune response is sufficient to inhibit, neutralise or prevent infection, and/or progress of infection, of more than one type of β-coronavirus (for example, SARS-CoV, and SARS-CoV-2). Optionally a broadly neutralising immune response is sufficient to inhibit, neutralise or prevent infection, and/or progress of infection, of more than one type of β-coronavirus within the same β-coronavirus lineage (for example, more than one type of β-coronavirus within the subgenus Sarbecovirus, such as SARS-CoV, SARS-CoV-2, and Bat SL-CoV-WIV1). Optionally a broadly neutralising immune response is sufficient to inhibit, neutralise or prevent infection, and/or progress of infection, of coronaviruses of different β-coronavirus lineages, such as lineage B (for example, SARS-CoV, and SARS-CoV-2) and lineage C (for example, MERS-COV). Optionally a broadly neutralising immune response is sufficient to inhibit, neutralise or prevent infection, and/or progress of infection, of most or all different β-coronaviruses. Optionally a broadly neutralising immune response is sufficient to inhibit, neutralise or prevent infection, and/or progress of infection, of most or all different viruses of the coronavirus family. Optionally a broadly neutralising immune response is sufficient to inhibit, neutralise or prevent infection, and/or progress of infection, of more than one type of β-coronavirus SARS-CoV-2 variant of concern (VOC), for example more than one of an alpha, beta, gamma, delta, omicron SARS-CoV-2 VOC.

The immune response may be humoral and/or a cellular immune response. A cellular immune response is a response of a cell of the immune system, such as a β-cell, T-cell, macrophage or polymorphonucleocyte, to a stimulus such as an antigen or vaccine. An immune response can include any cell of the body involved in a host defence response, including for example, an epithelial cell that secretes an interferon or a cytokine. An immune response includes, but is not limited to, an innate immune response or inflammation.

Optionally a polypeptide of the invention induces a protective immune response. A protective immune response refers to an immune response that protects a subject from infection or disease (i.e. prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, or antibody production.

Optionally a polypeptide of the invention is able to induce the production of antibodies and/or a T-cell response in a human or non-human animal to which the polypeptide has been administered (either as a polypeptide or, for example, expressed from an administered nucleic acid expression vector).

Optionally a polypeptide of the invention is a glycosylated polypeptide.

### Nucleic Acid Molecules

According to the invention there is also provided an isolated nucleic acid molecule encoding a polypeptide of the invention, or the complement thereof.

There is also provided according to the invention an isolated nucleic acid molecule comprising a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length to a nucleic acid molecule of the invention encoding a polypeptide of the invention, or the complement thereof.

Optionally an isolated nucleic acid molecule of the invention comprises a nucleotide sequence of SEQ ID NO: 18, 16, or 14, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with a nucleotide sequence of SEQ ID NO: 18, 16, or 14 over its entire length, or the complement thereof.

According to the invention there is also provided an isolated nucleic acid molecule which comprises a nucleotide sequence encoding a polypeptide of the invention comprising an amino acid sequence of SEQ ID NO:33, 34, 35, or 36.

Optionally the nucleotide sequence encoding a polypeptide comprising an amino acid sequence of SEQ ID NO:33, 34, 35, or 36 comprises a nucleotide sequence of SEQ ID NO:37, 38, 39, or 40, respectively.

According to the invention there is also provided an isolated nucleic acid molecule which comprises a nucleotide sequence encoding an isolated polypeptide of the invention comprising an amino acid sequence of SEQ ID NO: 34 (M8), or an amino acid sequence which has at least 99% amino acid identity over its entire length with the amino acid sequence of SEQ ID NO:34.

According to the invention there is also provided an isolated nucleic acid molecule which comprises a nucleotide sequence encoding an isolated polypeptide which comprises a coronavirus S protein RBD domain with at least one of the following amino acid residues at positions corresponding to the amino acid residue positions of SEQ ID NO:11: 13Q, 25Q, 54T, 203N.

According to the invention there is also provided an isolated nucleic acid molecule which comprises a nucleotide sequence encoding an isolated polypeptide comprising an amino acid sequence of SEQ ID NO: 35 (M9), or an amino acid sequence which has at least 70% amino acid identity over its entire length with the amino acid sequence of SEQ ID NO:35.

According to the invention there is also provided an isolated nucleic acid molecule which comprises a nucleotide sequence encoding an isolated polypeptide comprising an amino acid sequence of SEQ ID NO: 36 (M10), or an amino acid sequence which has at least 69% amino acid identity over its entire length with the amino acid sequence of SEQ ID NO:36.

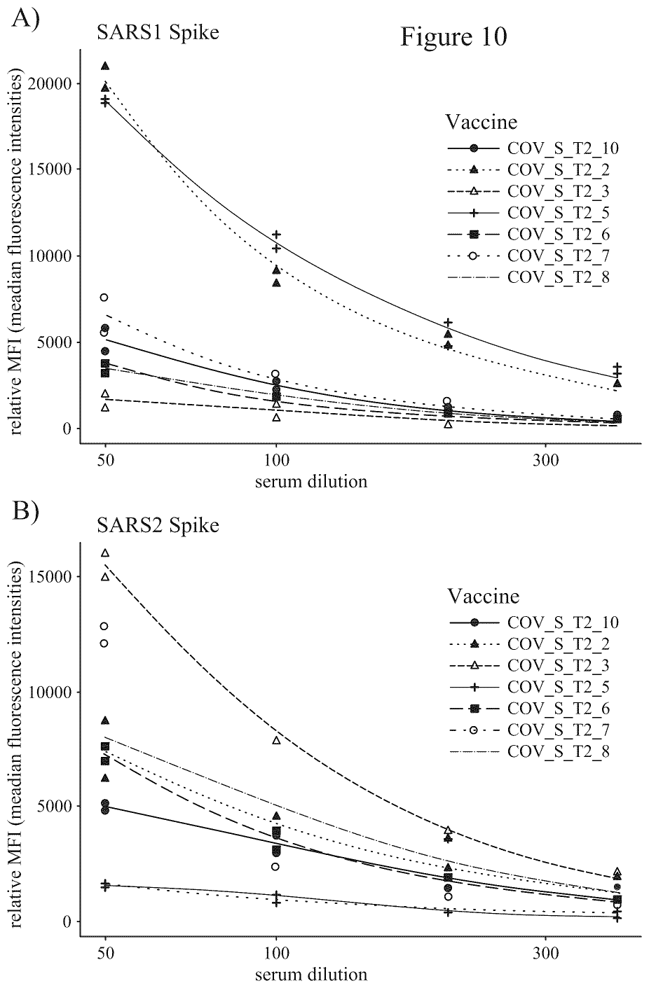

We have found that immunisation of mice with nucleic acid (in particular, DNA) encoding SARS2 truncated S protein induces production of antibodies that are able to bind SARS2 spike protein (see Example 17, FIG. 10).

According to the invention there is provided an isolated nucleic acid molecule encoding a SARS2 truncated S protein of amino acid sequence SEQ ID NO:9 (COV_T2_3).

Optionally the isolated nucleic acid molecule encoding a SARS2 truncated S protein of amino acid sequence SEQ ID NO:9 (COV_T2_3) comprises a nucleotide sequence of SEQ ID NO: 10.

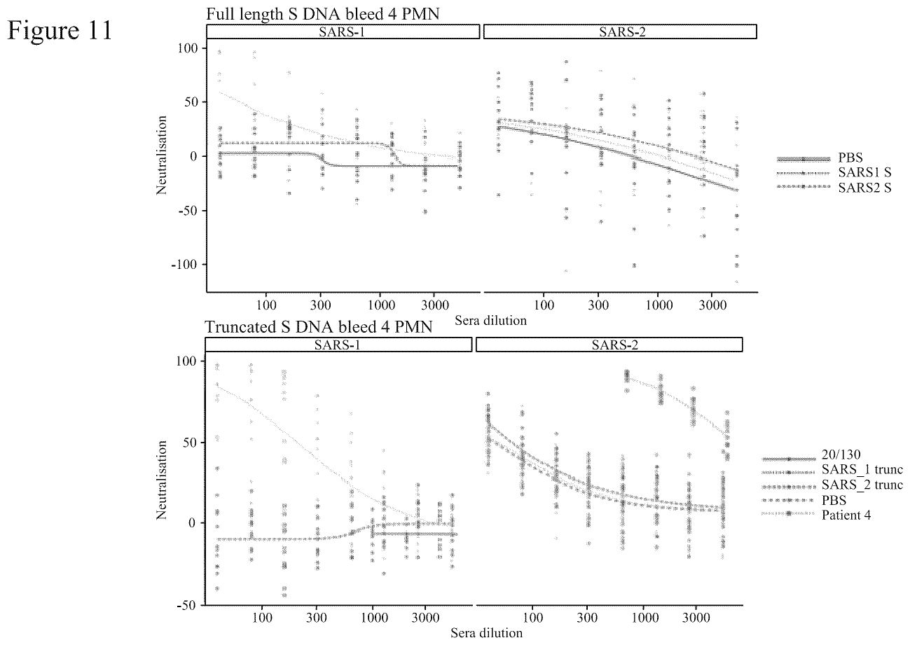

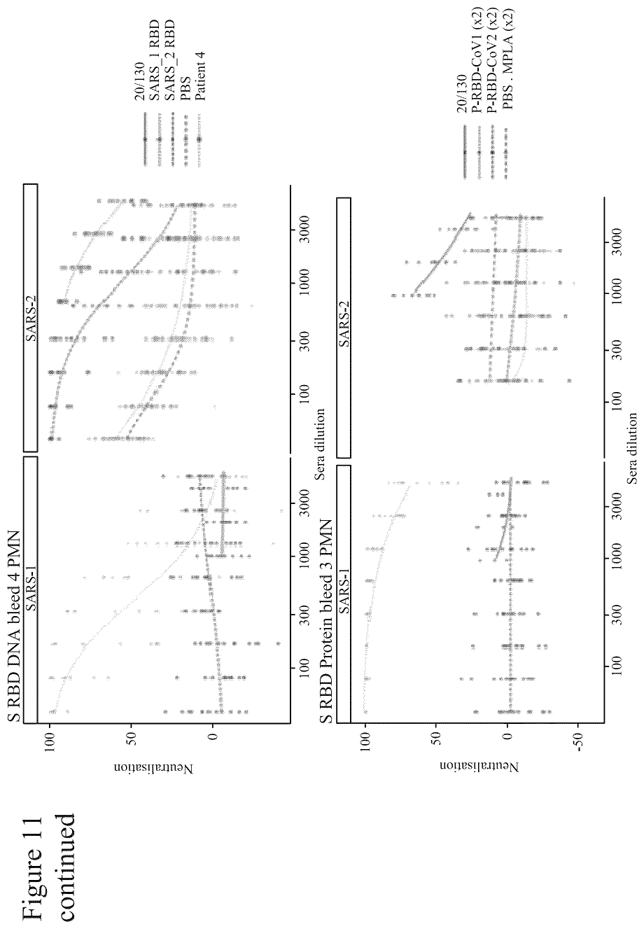

We have also found that immunisation of mice with nucleic acid (in particular, DNA) encoding SARS2 S protein RBD induces production of antibodies that are able to neutralise SARS2 pseudotype virus (see Example 18, FIG. 11).

We have also found that M7 and wild-type SARS2 RBD DNA (believed to result in expression of glycosylated RBD protein) is superior to recombinant SARS2 RBD protein (non-glycosylated, or sparsely glycosylated) in inducing neutralising responses to SARS2.

According to the invention there is provided an isolated nucleic acid molecule encoding a SARS2 S protein RBD of amino acid sequence SEQ ID NO: 11 (COV_T2_6).

Optionally the isolated nucleic acid molecule encoding a SARS2 S protein RBD of amino acid sequence SEQ ID NO: 11 (COV_T2_6) comprises a nucleotide sequence of SEQ ID NO: 12.

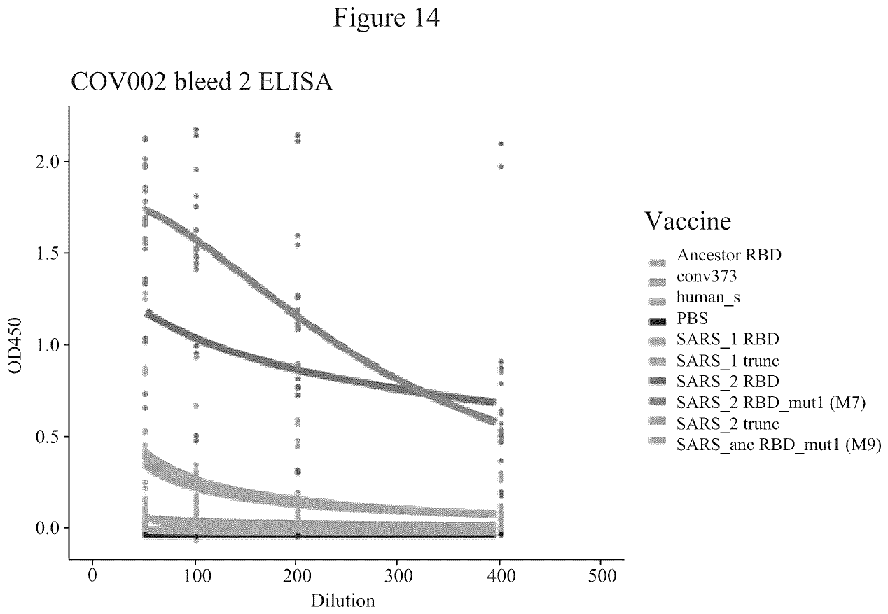

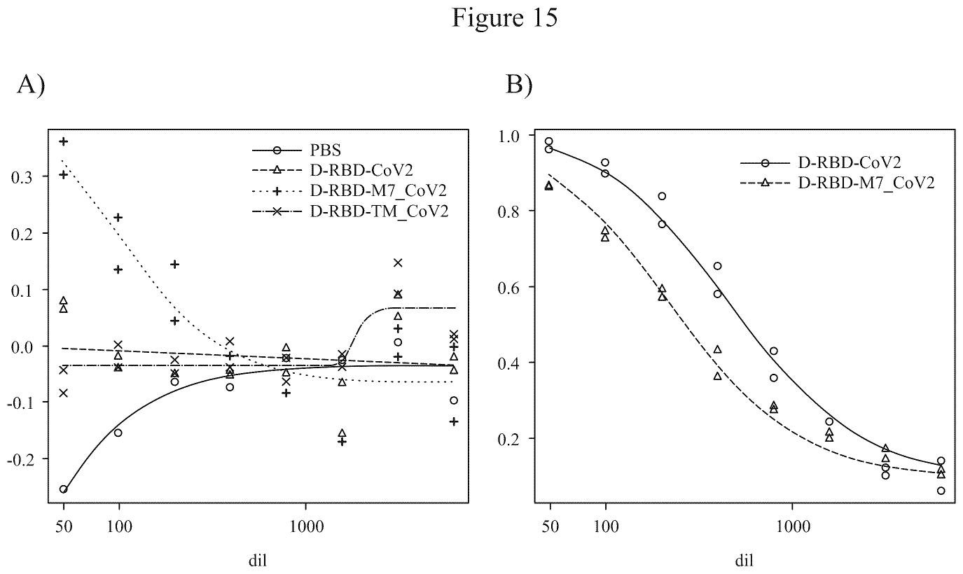

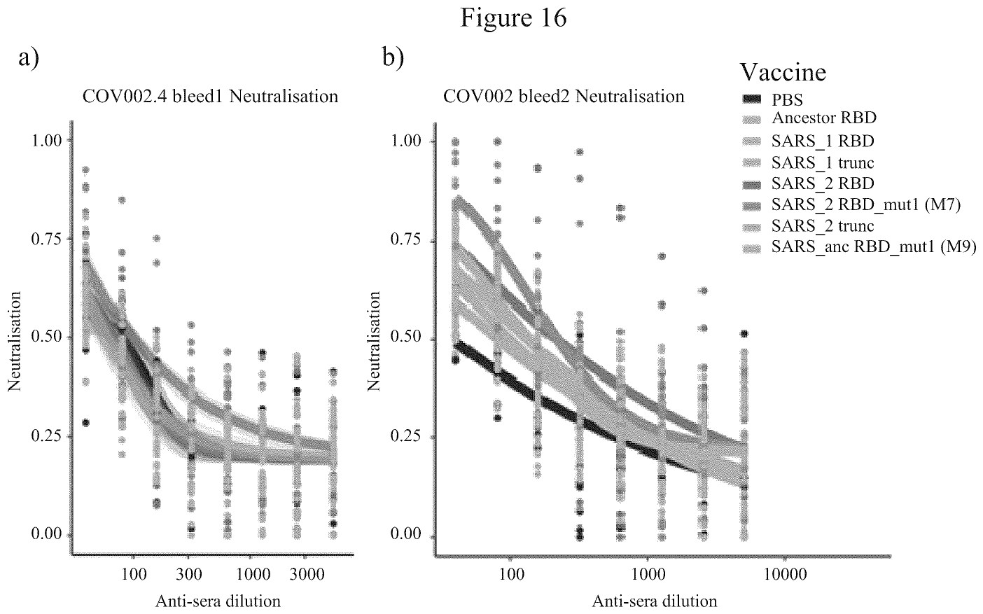

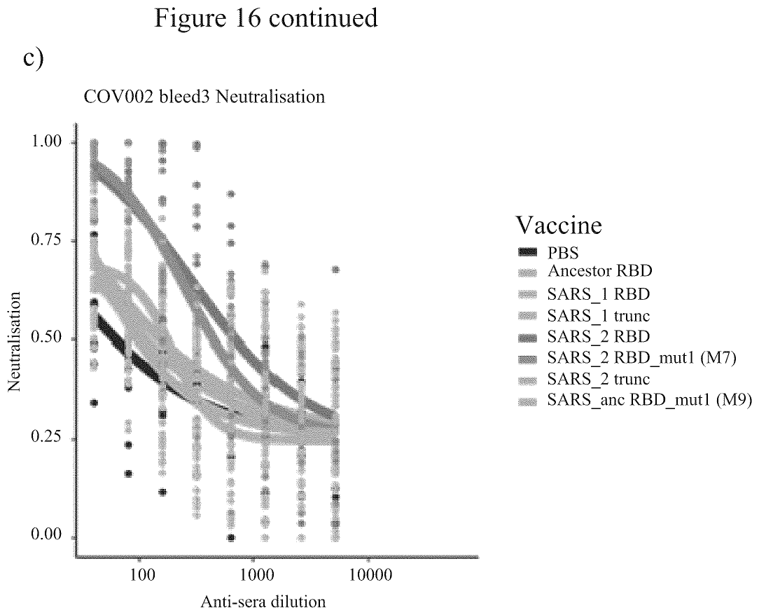

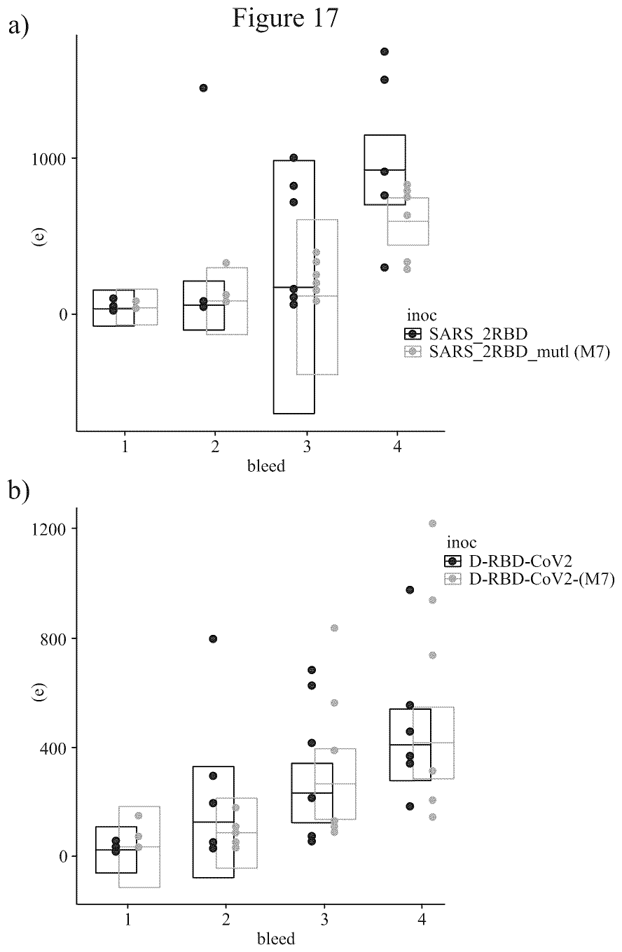

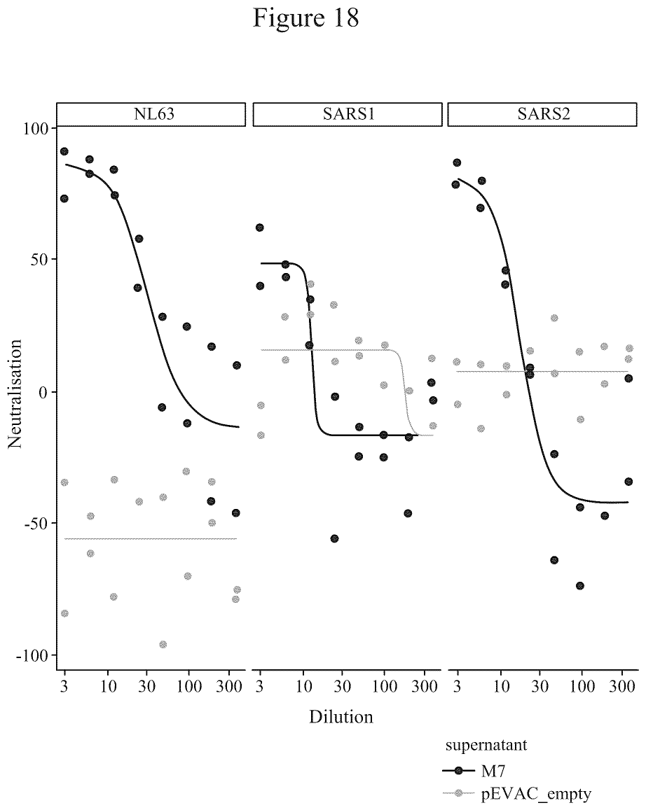

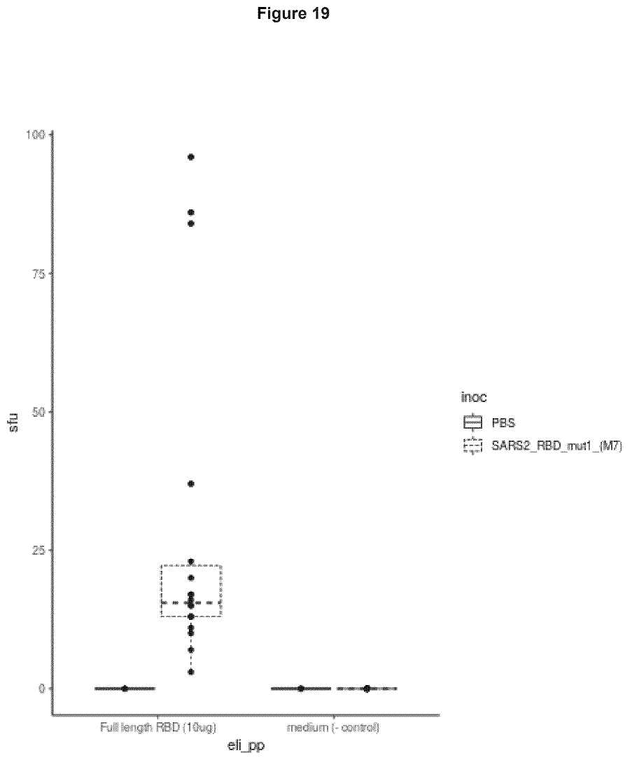

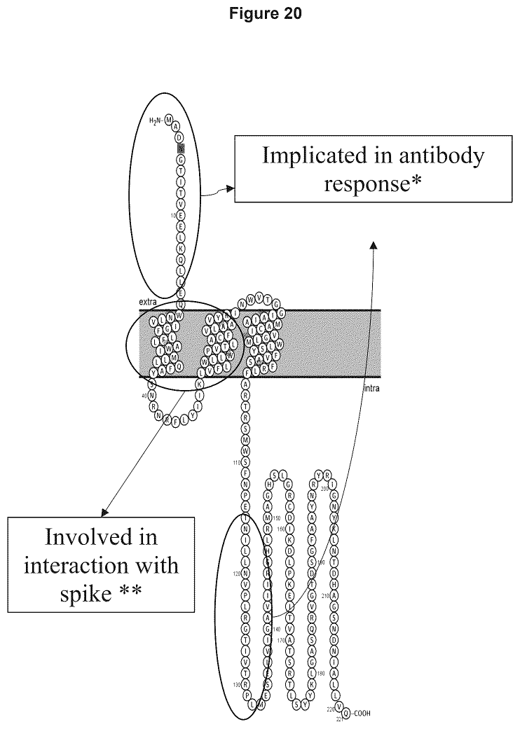

We have also found that nucleic acid (in particular, DNA) encoding the designed M7 SARS2 S protein RBD has especially advantageous effects. In particular, we have found that:
immunisation of mice with a DNA vaccine comprising nucleic acid encoding M7 SARS2 RBD (SEQ ID NO:33) induced an immune response with stronger binding to SARS2 RBD than wild-type SARS2 RBD (see Example 20, and FIG. 14);
immunisation of mice with a DNA vaccine encoding M7 SARS2 RBD (SEQ ID NO:33) elicits a neutralising immune response more rapidly than a DNA vaccine encoding wild-type SARS2 RBD (see Example 21, and FIG. 15);
immunisation of mice with a DNA vaccine encoding M7 SARS2 RBD (SEQ ID NO:33) induces a more neutralising response than a DNA vaccine encoding wild-type SARS2 RBD in sera collected from bleeds at weeks 1 and 2 (see Example 22, and FIGS. 16, 17);
supernatant comprising M7 SARS2 RBD competes effectively with three ACE2 binding viruses for ACE2 cell entry (see Example 23, and FIG. 18); and
T cell responses were induced by a DNA vaccine encoding M7 SARS2 RBD (SEQ ID NO: 33) that were reactive against peptides of an RBD peptide pool, but not against full length RBD or medium (see Example 24, and FIG. 19).

There is also provided according to the invention an isolated nucleic acid molecule comprising a nucleotide sequence of SEQ ID NO:37.

There is also provided according to the invention an isolated nucleic acid molecule comprising a nucleotide sequence of SEQ ID NO:78 (nucleic acid encoding COV_S_T2_13).

There is also provided according to the invention an isolated nucleic acid molecule comprising a nucleotide sequence of SEQ ID NO:79 (nucleic acid encoding COV_S_T2_14).

There is also provided according to the invention an isolated nucleic acid molecule comprising a nucleotide sequence of SEQ ID NO:80 (nucleic acid encoding COV_S_T2_15).

There is also provided according to the invention an isolated nucleic acid molecule comprising a nucleotide sequence of SEQ ID NO:81 (nucleic acid encoding COV_S_T2_16).

There is also provided according to the invention an isolated nucleic acid molecule comprising a nucleotide sequence of SEQ ID NO:82 (nucleic acid encoding COV_S_T2_17).

There is also provided according to the invention an isolated nucleic acid molecule comprising a nucleotide sequence of SEQ ID NO:83 (nucleic acid encoding COV_S_T2_18).

There is also provided according to the invention an isolated nucleic acid molecule comprising a nucleotide sequence of SEQ ID NO:84 (nucleic acid encoding COV_S_T2_19).

There is also provided according to the invention an isolated nucleic acid molecule comprising a nucleotide sequence of SEQ ID NO:85 (nucleic acid encoding COV_S_T2_20).

There is also provided according to the invention an isolated nucleic acid molecule comprising a nucleotide sequence of SEQ ID NO:86 (T2_17+pEVAC Expression Vector).

There is also provided according to the invention an isolated nucleic acid molecule comprising a nucleotide sequence encoding an amino acid sequence of SEQ ID NO:92 (CoV_S_T2_17+tPA signal sequence).

There is also provided according to the invention an isolated nucleic acid molecule comprising a nucleotide sequence of SEQ ID NO:93 (COV_S_T2_17+tPA signal sequence).

There is also provided according to the invention an isolated nucleic acid molecule comprising a nucleotide sequence of SEQ ID NO:94 (pURVAC_T2_17+tPA).

There is also provided according to the invention an isolated nucleic acid molecule comprising a nucleotide sequence of SEQ ID NO:95 (pURVAC_COV_S_T2_29+Q498R+dER).

There is also provided according to the invention an isolated nucleic acid molecule comprising a nucleotide sequence of SEQ ID NO:97 (pMVA Trans TK mH5 T2_17+tPA).

There is also provided according to the invention an isolated nucleic acid molecule comprising a nucleotide sequence of SEQ ID NO:98 (pMVA Trans TK mH5 T2_29+Q498R+dER).

### Sequence Identity

The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a given gene or protein will possess a relatively high degree of sequence identity when aligned using standard methods. Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-244, 1988; Higgins and Sharp, CABIOS 5:151-153, 1989; Corpet et al., Nucleic Acids' Research 16:10881-10890, 1988; and Pearson and Lipman, Proc. Natl. Acad. Sci. U.S.A. 85:2444, 1988. Altschul et al., Nature Genet. 6:119-129, 1994. The NCBI Basic Local Alignment Search Tool (BLAST™) (Altschul et al., J. Mol. Biol. 215:403-410, 1990) is available from several sources, including the National Center for Biotechnology Information (NCBI, Bethesda, MD) and on the Internet, for use in connection with the sequence analysis programs blastp, blastn, blastx, tblastn and tblastx.

Sequence identity between nucleic acid sequences, or between amino acid sequences, can be determined by comparing an alignment of the sequences. When an equivalent position in the compared sequences is occupied by the same nucleotide, or amino acid, then the molecules are identical at that position. Scoring an alignment as a percentage of identity is a function of the number of identical nucleotides or amino acids at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4:29; program available from http://bitincka.com/ledion/matgat), Gap (Needleman & Wunsch, 1970, J. Mol. Biol. 48:443-453), FASTA (Altschul et al., 1990, J. Mol. Biol. 215: 403-410; program available from http://www.ebi.ac.uk/fasta), Clustal W 2.0 and X 2.0 (Larkin et al., 2007, Bioinformatics 23:2947-2948; program available from http://www.ebi.ac.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from_http://www.ebi.ac.uk/tools/emboss/align). All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score. Default parameters for amino acid sequence comparisons (“Protein Molecule” option) may be Gap Extend penalty: 0.5, Gap Open penalty: 10.0, Matrix: Blosum 62.

The sequence comparison may be performed over the full length of the reference sequence.

### Corresponding Positions

Sequences described herein include reference to an amino acid sequence comprising an amino acid residue “at a position corresponding to an amino acid residue position” of another sequence. Such corresponding positions may be identified, for example, from an alignment of the sequences using a sequence alignment method described herein, or another sequence alignment method known to the person of ordinary skill in the art.

### Vectors

There is also provided according to the invention a vector comprising a nucleic acid molecule of the invention.

There is also provided according to the invention a vector comprising a nucleic acid molecule encoding a polypeptide of the invention.

Optionally a vector of the invention comprises a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17.

Optionally a vector of the invention comprises a nucleic acid molecule encoding a polypeptide of the invention which comprises an amino acid sequence of SEQ ID NO: 15, or an amino acid sequence which has at least 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 15.

Optionally a vector of the invention comprises a nucleic acid molecule encoding a polypeptide of the invention which comprises an amino acid sequence of SEQ ID NO: 13, or an amino acid sequence which has at least 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 13.

Optionally a vector of the invention comprises a nucleic acid molecule encoding a polypeptide of the invention which comprises an amino acid sequence of SEQ ID NO: 27 (COV_S_T2_13), or an amino acid sequence which has at least 88%, 89%, 90%, 91%, 92%, 93%, 94%, 95%, 96%, 97%, 98%, or 99% amino acid identity over its entire length with the amino acid sequence of SEQ ID NO:27.

Optionally a vector of the invention comprises a nucleic acid molecule encoding a polypeptide of the invention which comprises an amino acid sequence of SEQ ID NO: 28 (COV_S_T2_14), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28.

Optionally a vector of the invention comprises a nucleic acid molecule encoding a polypeptide of the invention which comprises an amino acid sequence of SEQ ID NO: 29 (COV_S_T2_15), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29.

Optionally a vector of the invention comprises a nucleic acid molecule encoding a polypeptide of the invention which comprises an amino acid sequence of SEQ ID NO: 30 (COV_S_T2_16), or an amino acid sequence which has at least 93%, 94%, 95%, 96%, 97%, 98%, or 99% amino acid identity over its entire length with the amino acid sequence of SEQ ID NO:30.

Optionally a vector of the invention comprises a nucleic acid molecule encoding a polypeptide of the invention which comprises an amino acid sequence of SEQ ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31.

Optionally a vector of the invention comprises a nucleic acid molecule encoding a polypeptide of the invention which comprises an amino acid sequence of SEQ ID NO: 32 (COV_S_T2_18), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32.

Optionally a vector of the invention comprises a nucleic acid molecule encoding a polypeptide of the invention which comprises an amino acid sequence of SEQ ID NO: 33.

Optionally a vector of the invention comprises a nucleic acid molecule encoding a polypeptide of the invention which comprises an amino acid sequence of SEQ ID NO: 34, or an amino acid sequence which has at least 99% amino acid identity over its entire length with the amino acid sequence of SEQ ID NO:34.

Optionally a vector of the invention comprises a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22.

Optionally a vector of the invention comprises a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23.

Optionally a vector of the invention comprises a nucleic acid molecule encoding a polypeptide of the invention which comprises an amino acid sequence of SEQ ID NO:42 (COV_E_T2_3), or an amino acid sequence which has at least 99% amino acid identity over its entire length with the amino acid sequence of SEQ ID NO:42.

Optionally a vector of the invention comprises a nucleic acid molecule encoding a polypeptide of the invention which comprises an amino acid sequence of SEQ ID NO:43 (COV_E_T2_4), or an amino acid sequence which has at least 95%, 96%, 97%, 98%, or 99% amino acid identity over its entire length with the amino acid sequence of SEQ ID NO:43.

Optionally a vector of the invention comprises a nucleic acid molecule encoding a polypeptide of the invention which comprises an amino acid sequence of SEQ ID NO:44 (COV_E_T2_5), or an amino acid sequence which has at least 98%, or 99% amino acid identity over its entire length with the amino acid sequence of SEQ ID NO:44.

Optionally a vector of the invention comprises a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24.

Optionally a vector of the invention comprises a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally a vector of the invention comprises a nucleic acid molecule encoding a polypeptide of the invention which comprises an amino acid sequence of SEQ ID NO:46 (COV_N_T2_1), or an amino acid sequence which has at least 93%, 94%, 95%, 96%, 97%, 98%, or 99% amino acid identity over its entire length with the amino acid sequence of SEQ ID NO:46.

Optionally a vector of the invention comprises a nucleic acid molecule encoding a polypeptide of the invention which comprises an amino acid sequence of SEQ ID NO:47 (COV_N_T2_2), or an amino acid sequence which has at least 92%, 93%, 94%, 95%, 96%, 97%, 98%, or 99% amino acid identity over its entire length with the amino acid sequence of SEQ ID NO:47.

Optionally a vector of the invention further comprises a promoter operably linked to the nucleic acid.

Optionally the promoter is for expression of a polypeptide encoded by the nucleic acid in mammalian cells.

Optionally the promoter is for expression of a polypeptide encoded by the nucleic acid in yeast or insect cells.

Optionally a vector of the invention comprises more than one nucleic acid molecule encoding a different polypeptide of the invention. Advantageously, a vector of the invention comprises a nucleic acid molecule of the invention encoding a designed coronavirus S protein (full length, truncated, or RBD) of the invention and/or a nucleic acid molecule of the invention encoding a designed coronavirus E protein of the invention and/or a nucleic acid molecule of the invention encoding a designed coronavirus M protein of the invention.

Optionally a vector of the invention comprises more than one nucleic acid molecule encoding a different polypeptide of the invention. Advantageously, a vector of the invention comprises a nucleic acid molecule of the invention encoding a designed coronavirus S protein (full length, truncated, or RBD) of the invention and/or a nucleic acid molecule of the invention encoding a designed coronavirus E protein of the invention and/or a nucleic acid molecule of the invention encoding a designed coronavirus M protein of the invention and/or a nucleic acid molecule of the invention encoding a designed coronavirus N protein of the invention

Optionally a vector of the invention comprises a nucleic acid molecule of the invention encoding a designed coronavirus S protein (full length, truncated, or RBD) of the invention and a nucleic acid molecule of the invention encoding a designed coronavirus E protein of the invention.

Optionally a vector of the invention comprises a nucleic acid molecule of the invention encoding a designed coronavirus S protein (full length, truncated, or RBD) of the invention and a nucleic acid molecule of the invention encoding a designed coronavirus M protein of the invention.

Optionally a vector of the invention comprises a nucleic acid molecule of the invention encoding a designed coronavirus S protein (full length, truncated, or RBD) of the invention and a nucleic acid molecule of the invention encoding a designed coronavirus N protein of the invention.

Optionally a vector of the invention comprises a nucleic acid molecule of the invention encoding a designed coronavirus E protein of the invention and a nucleic acid molecule of the invention encoding a designed coronavirus M protein of the invention.

Optionally a vector of the invention comprises a nucleic acid molecule of the invention encoding a designed coronavirus E protein of the invention and a nucleic acid molecule of the invention encoding a designed coronavirus N protein of the invention.

Optionally a vector of the invention comprises a nucleic acid molecule of the invention encoding a designed coronavirus S protein (full length, truncated, or RBD) of the invention and a nucleic acid molecule of the invention encoding a designed coronavirus E protein of the invention and a nucleic acid molecule of the invention encoding a designed coronavirus M protein of the invention.

Optionally a vector of the invention comprises a nucleic acid molecule of the invention encoding a designed coronavirus S protein (full length, truncated, or RBD) of the invention and a nucleic acid molecule of the invention encoding a designed coronavirus E protein of the invention and a nucleic acid molecule of the invention encoding a designed coronavirus N protein of the invention.

Optionally a vector of the invention comprises a nucleic acid molecule of the invention encoding a designed coronavirus E protein of the invention and a nucleic acid molecule of the invention encoding a designed coronavirus M protein of the invention and a nucleic acid molecule of the invention encoding a designed coronavirus N protein of the invention.

Optionally a vector of the invention comprises a nucleic acid molecule of the invention encoding a designed coronavirus S protein (full length, truncated, or RBD) of the invention and a nucleic acid molecule of the invention encoding a designed coronavirus E protein of the invention and a nucleic acid molecule of the invention encoding a designed coronavirus M protein of the invention and a nucleic acid molecule of the invention encoding a designed coronavirus N protein of the invention.

Optionally a vector of the invention comprises:
a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17; and
a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23.

Optionally a vector of the invention comprises:
a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17; and
a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally a vector of the invention comprises:
a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23; and
a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally a vector of the invention comprises:
a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17; and
a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23; and
a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally a vector of the invention which further comprises, for each nucleic acid molecule of the vector encoding a polypeptide, a separate promoter operably linked to that nucleic acid molecule.

Optionally the, or each promoter is for expression of a polypeptide encoded by the nucleic acid molecule in mammalian cells.

Optionally the, or each promoter is for expression of a polypeptide encoded by the nucleic acid molecule in yeast or insect cells.

Optionally the vector is a vaccine vector.

Optionally the vector is a viral vaccine vector, a bacterial vaccine vector, an RNA vaccine vector, or a DNA vaccine vector.

A nucleic acid molecule of the invention may comprise a DNA or an RNA molecule. For embodiments in which the nucleic acid comprises an RNA molecule, it will be appreciated that the nucleic acid sequence of the nucleic acid will be the same as that recited in the respective SEQ ID, or the complement thereof, but with each ‘T’ nucleotide replaced by ‘U’.

For embodiments in which the nucleic acid molecule comprises an RNA molecule, it will be appreciated that the molecule may comprise an RNA sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with, or identical with, any of SEQ ID NOs: 18, 16, or 14, in which each ‘T’ nucleotide is replaced by ‘U’, or the complement thereof.

For example, it will be appreciated that where an RNA vaccine vector comprising a nucleic acid of the invention is provided, the nucleic acid sequence of the nucleic acid of the invention will be an RNA sequence, so may comprise for example an RNA nucleic acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with, or identical with, any of SEQ ID NOs: 18, 16, or 14 in which each ‘T’ nucleotide is replaced by ‘U’, or the complement thereof.

Viral vaccine vectors use live viruses to deliver nucleic acid (for example, DNA or RNA) into human or non-human animal cells. The nucleic acid contained in the virus encodes one or more antigens that, once expressed in the infected human or non-human animal cells, elicit an immune response. Both humoral and cell-mediated immune responses can be induced by viral vaccine vectors. Viral vaccine vectors combine many of the positive qualities of nucleic acid vaccines with those of live attenuated vaccines. Like nucleic acid vaccines, viral vaccine vectors carry nucleic acid into a host cell for production of antigenic proteins that can be tailored to stimulate a range of immune responses, including antibody, T helper cell (CD4+ T cell), and cytotoxic T lymphocyte (CTL, CD8+ T cell) mediated immunity. Viral vaccine vectors, unlike nucleic acid vaccines, also have the potential to actively invade host cells and replicate, much like a live attenuated vaccine, further activating the immune system like an adjuvant. A viral vaccine vector therefore generally comprises a live attenuated virus that is genetically engineered to carry nucleic acid (for example, DNA or RNA) encoding protein antigens from an unrelated organism. Although viral vaccine vectors are generally able to produce stronger immune responses than nucleic acid vaccines, for some diseases viral vectors are used in combination with other vaccine technologies in a strategy called heterologous prime-boost. In this system, one vaccine is given as a priming step, followed by vaccination using an alternative vaccine as a booster. The heterologous prime-boost strategy aims to provide a stronger overall immune response. Viral vaccine vectors may be used as both prime and boost vaccines as part of this strategy. Viral vaccine vectors are reviewed by Ura et al., 2014 (Vaccines 2014, 2, 624-641) and Choi and Chang, 2013 (Clinical and Experimental Vaccine Research 2013; 2:97-105).

Optionally the viral vaccine vector is based on a viral delivery vector, such as a Poxvirus (for example, Modified Vaccinia Ankara (MVA), NYVAC, AVIPOX), herpesvirus (e.g. HSV, CMV, Adenovirus of any host species), Morbillivirus (e.g. measles), Alphavirus (e.g. SFV, Sendai), Flavivirus (e.g. Yellow Fever), or Rhabdovirus (e.g. VSV)-based viral delivery vector, a bacterial delivery vector (for example, Salmonella, E. coli), an RNA expression vector, or a DNA expression vector.

Adenoviruses are by far the most utilised and advanced viral vectors developed for SARS2 vaccines. They are non-enveloped double-stranded DNA (dsDNA) viruses with a packaging capacity of up to 7.5 kb of foreign genes. Almost all SARS2 adenovirus based vaccines have been engineered for the expression of the SARS2 S protein or the RBD subunit. Recombinant Adenovirus vectors are widely used because of their high transduction efficiency, high level of transgene expression, and broad range of viral tropism. These vaccines are highly cell specific, highly efficient in gene transduction, and efficient at inducing an immune response. Adenovirus vaccines are effective at triggering and priming T cells, leading to long term and high level of antigenic protein expression and therefore long lasting protection. AZD1222 (manufactured by AstraZeneca) vaccine construct comprises a recombinant adenoviral vector vaccine encoding the SARS2 S protein. The recombinant adenovirus genome comprises SARS2 S gene at the E1 locus.

Optionally a vaccine of the invention (optionally a nucleic acid or polypeptide of the invention) is administered as part of a heterologous prime-boost regimen, for example using an heterologous DNA prime/MVA boost regimen.

Optionally a method of inducing an immune response to a coronavirus in a subject, or a method of immunising a subject against a coronavirus, according to the invention comprises administering a nucleic acid of the invention, a vector of the invention, or a pharmaceutical composition of the invention, wherein the nucleic acid, vector, or pharmaceutical composition is administered as part of a heterologous prime boost regimen.

Optionally the heterologous prime boost regimen comprises a prime with a DNA vector of the invention followed by a boost with an MVA vector of the invention.

Optionally the DNA prime comprises administration of a DNA vaccine vector comprising a nucleic acid molecule of the invention, and the MVA boost comprises administration of an MVA vector comprising a nucleic acid molecule of the invention, optionally wherein the nucleic acid molecule of the invention of the DNA vaccine vector encodes the same amino acid sequence as the nucleic acid molecule of the invention of the MVA vector.

For example, a nucleic acid molecule (optionally a DNA molecule) encoding a designed S protein RBD sequence M7 polypeptide of the invention (SEQ ID NO:33) may be administered as part of a prime-boost vaccination using an MVA boost. As shown in Example 38 below, a heterologous DNA prime/MVA boost M7 regimen induced higher, broadly neutralising, and long-lasting antibodies against variants of concern.

In a further example, a nucleic acid molecule (optionally a DNA molecule) encoding a designed S protein sequence T2_29 polypeptide of the invention (SEQ ID NO:88-COV_S_T2_29+Q498R+dER; COV_S_T2_29+Q498R-SEQ ID NO:87; or COV_S_T2_29-SEQ ID NO:53) may be administered as part of an heterologous prime-boost vaccination using an MVA boost. As shown in Example 37 below, a prime with DNA vector comprising DNA encoding amino acid sequence of SEQ ID NO:53, 87, or 88, followed by a boost with an MVA vector comprising nucleic acid encoding amino acid sequence of SEQ ID NO:88, induced broad neutralising response against all the VOCs tested—at least two-fold better neutralising response against Alpha, Beta, Gamma, and Omicron VOCs in comparison to WTdER after three doses of DNA vaccine.

In a further example, a nucleic acid molecule (optionally a DNA molecule) encoding a designed S protein sequence T2_17 polypeptide of the invention (SEQ ID NO:31) may be administered as part of an heterologous prime-boost vaccination using an MVA boost with an MVA vector comprising nucleic acid encoding amino acid sequence of SEQ ID NO:31.

Optionally the prime with a DNA vector of the invention may comprise administration of the DNA vector once, twice, or three times, prior to the MVA boost.

The MVA boost may be administered at least a day, at least a week, or at least two, three, four, five, six, or seven weeks, after the final administration of the DNA vector.

There is also provided according to the invention a kit comprising a DNA vaccine vector which comprises a nucleic acid molecule of the invention, and an MVA vector which comprises a nucleic acid molecule of the invention, optionally wherein the nucleic acid molecule of the invention of the DNA vaccine vector encodes the same amino acid sequence as the nucleic acid molecule of the invention of the MVA vector.

Optionally the nucleic acid molecule of the invention of the DNA vaccine vector encodes a designed S protein sequence T2_29 polypeptide of the invention (SEQ ID NO:88-COV_S_T2_29+Q498R+dER; COV_S_T2_29+Q498R-SEQ ID NO:87; or COV_S_T2_29-SEQ ID NO:53), and the nucleic acid molecule of the invention of the MVA vector encodes an amino acid sequence of SEQ ID NO:88.

Optionally the nucleic acid molecule of the invention of the DNA vaccine vector encodes an amino acid sequence of SEQ ID NO:33, and the nucleic acid molecule of the invention of the MVA vector encodes an amino acid sequence of SEQ ID NO:33.

Optionally the nucleic acid molecule of the invention of the DNA vaccine vector encodes an amino acid sequence of SEQ ID NO:31, and the nucleic acid molecule of the invention of the MVA vector encodes an amino acid sequence of SEQ ID NO:31.

Optionally the nucleic acid expression vector is a nucleic acid expression vector, and a viral pseudotype vector.

Optionally the nucleic acid expression vector is a vaccine vector.

Optionally the nucleic acid expression vector comprises, from a 5′ to 3′ direction: a promoter; a splice donor site (SD); a splice acceptor site (SA); and a terminator signal, wherein the multiple cloning site is located between the splice acceptor site and the terminator signal.

Optionally the promoter comprises a CMV immediate early 1 enhancer/promoter (CMV-IE-E/P) and/or the terminator signal comprises a terminator signal of a bovine growth hormone gene (Tbgh) that lacks a Kpnl restriction endonuclease site.

Optionally the nucleic acid expression vector further comprises an origin of replication, and nucleic acid encoding resistance to an antibiotic. Optionally the origin of replication comprises a pUC-plasmid origin of replication and/or the nucleic acid encodes resistance to kanamycin.

Optionally the vector is a pEVAC-based expression vector.

Optionally the nucleic acid expression vector comprises a nucleic acid sequence of SEQ ID NO: 20 (pEVAC). The pEVAC vector has proven to be a highly versatile expression vector for generating viral pseudotypes as well as direct DNA vaccination of animals and humans. The pEVAC expression vector is described in more detail in Example 8 below. FIG. 3 shows a plasmid map for pEVAC.

The terms “polynucleotide” and “nucleic acid” are used interchangeably herein.

A polynucleotide (or nucleic acid) of the invention may comprise a DNA molecule.

The or each polynucleotide (or nucleic acid) of a pharmaceutical composition, a combined preparation, or a vector, of the invention may comprise a DNA molecule.

A vector of the invention may be a DNA vector.

The or each vector of a pharmaceutical composition or a combined preparation of the invention may be a DNA vector.

A polynucleotide (or nucleic acid) of the invention, or a polynucleotide (or nucleic acid) of a pharmaceutical composition, a combined preparation, or a vector, of the invention, may be provided as part of a DNA vaccine.

There is also provided according to the invention a DNA vaccine which comprises a polynucleotide (or nucleic acid) of the invention, a vector of the invention, or a pharmaceutical composition or a combined preparation of the invention which comprises one or more polynucleotides (or nucleic acids), wherein the or each polynucleotide (or nucleic acid) is a DNA molecule.

Optionally the, or each vaccine vector is an RNA vaccine vector.

A polynucleotide (or nucleic acid) of the invention may comprise an RNA molecule.

The or each polynucleotide (or nucleic acid) of a pharmaceutical composition, a combined preparation, or a vector, of the invention may comprise an RNA molecule.

A vector of the invention may be an RNA vector.

The or each vector of a pharmaceutical composition or a combined preparation of the invention may be an RNA vector.

A polynucleotide (or nucleic acid) of the invention, or a polynucleotide (or nucleic acid) of a pharmaceutical composition, a combined preparation, or a vector, of the invention, may be provided as part of an RNA vaccine.

There is also provided according to the invention an RNA vaccine which comprises a polynucleotide (or nucleic acid) of the invention, a vector of the invention, or a pharmaceutical composition or a combined preparation of the invention which comprises one or more polynucleotides (or nucleic acids), wherein the or each polynucleotide (or nucleic acid) is an RNA molecule.

A polynucleotide (or nucleic acid) of the invention may comprise an mRNA molecule.

The or each polynucleotide (or nucleic acid) of a pharmaceutical composition, a combined preparation, or a vector, of the invention may comprise an mRNA molecule.

A vector of the invention may be an mRNA vector.

Optionally the, or each vaccine vector is an mRNA vaccine vector.

The or each vector of a pharmaceutical composition or a combined preparation of the invention may be an mRNA vector.

A polynucleotide (or nucleic acid) of the invention, or a polynucleotide (or nucleic acid) of a pharmaceutical composition, a combined preparation, or a vector, of the invention, may be provided as part of an mRNA vaccine.

There is also provided according to the invention an mRNA vaccine which comprises a polynucleotide (or nucleic acid) of the invention, a vector of the invention, or a pharmaceutical composition or a combined preparation of the invention which comprises one or more polynucleotides (or nucleic acids), wherein the or each polynucleotide (or nucleic acid) comprises an mRNA molecule.

Messenger RNA (mRNA) vaccines are a new form of vaccine (recently reviewed in Pardi et al., Nature Reviews Drug Discovery Volume 17, pages 261-279 (2018); Wang et al., Molecular Cancer (2021) 20:33: mRNA vaccine: a potential therapeutic strategy). The first mRNA vaccines to be approved for use were BNT162b2 (manufactured by Pfizer) and mRNA-1273 (manufactured by Moderna) during the COVID-19 pandemic. mRNA vaccines have a unique feature of temporarily promoting the expression of antigen (typically days). The expression of the exogenous antigen is controlled by the lifetime of encoding mRNA, which is regulated by cellular degradation pathways. While this transient nature of protein expression requires repeated administration for the treatment of genetic diseases and cancers, it is extremely beneficial for vaccines, where prime or prime-boost vaccination is sufficient to develop highly specific adaptive immunity without any exposure to the contagion.

mRNA based vaccines trigger an immune response after the synthetic mRNA which encodes viral antigens transfects human cells. The cytosolic mRNA molecules are then translated by the host's own cellular machinery into specific viral antigens. These antigens may then be presented on the cell surface where they can be recognised by immune cells, triggering an immune response.

The structural elements of a vaccine vector mRNA molecule are similar to those of natural mRNA, comprising a 5′ cap, 5′ untranslated region (UTR), coding region (for example, comprising an open reading frame encoding a polypeptide of the invention), 3′ UTR, and a poly(A) tail. The 5′ UTR (also known as a leader sequence, transcript leader, or leader RNA) is the region of an mRNA that is directly upstream from the initiation codon. This region is important for the regulation of translation of a transcript. In many organisms, the 5′ UTR forms complex secondary structure to regulate translation. The 5′ UTR begins at the transcription start site and ends one nucleotide (nt) before the initiation sequence (usually AUG) of the coding region. In eukaryotes, the length of the 5′ UTR tends to be anywhere from 100 to several thousand nucleotides long. The differing sizes are likely due to the complexity of the eukaryotic regulation which the 5′ UTR holds as well as the larger pre-initiation complex that must form to begin translation. The eukaryotic 5′ UTR contains the Kozak consensus sequence (ACCAUG (initiation codon underlined), which contains the initiation codon AUG. An elongated Kozak sequence may be used: GCCACCAUG (initiation codon underlined).

Two major types of RNA are currently studied as vaccines: non-replicating mRNA and virally derived, self-amplifying RNA. While both types of vaccines share a common structure in mRNA constructs, self-amplifying RNA vaccines contain additional sequences in the coding region for RNA replication, including RNA-dependent RNA polymerases.

BNT162b2 vaccine construct comprises a lipid nanoparticle (LNP) encapsulated mRNA molecule encoding trimerised full-length SARS2 S protein with a PP mutation (at residue positions 986-987). The mRNA is encapsulated in 80 nm ionizable cationic lipid nanoparticles. mRNA-1273 vaccine construct is also based on an LNP vector, but the synthetic mRNA encapsulated within the lipid construct encodes the full-length SARS2 S protein.

U.S. Pat. No. 10,702,600 B1 (ModernaTX) describes betacoronavirus mRNA vaccines, including suitable LNPs for use in such vaccines.

A nucleic acid vaccine (for example, a mRNA) of the invention may be formulated in a lipid nanoparticle.

mRNA vaccines have several advantages in comparison with conventional vaccines containing inactivated (or live attenuated) disease-causing organisms. Firstly, mRNA-based vaccines can be rapidly developed due to design flexibility and the ability of the constructs to mimic antigen structure and expression as seen in the course of a natural infection. mRNA vaccines can be developed within days or months based on sequencing information from a target virus, while conventional vaccines often take years and require a deep understanding of the target virus to make the vaccine effective and safe. Secondly, these novel vaccines can be rapidly produced. Due to high yields from in vitro transcription reactions, mRNA production can be rapid, inexpensive and scalable. Thirdly, vaccine risks are low. mRNA does not contain infectious viral elements that pose risks for infection and insertional mutagenesis. Anti-vector immunity is also avoided as mRNA is the minimally immunogenic genetic vector, allowing repeated administration of the vaccine. The challenge for effective application of mRNA vaccines lies in cytosolic delivery. mRNA isolates are rapidly degraded by extracellular RNases and cannot penetrate cell membranes to be transcribed in the cytosol. However, efficient in vivo delivery can be achieved by formulating mRNA into carrier molecules, allowing rapid uptake and expression in the cytoplasm. To date, numerous delivery methods have been developed including lipid-, polymer-, or peptide-based delivery, virus-like replicon particle, cationic nanoemulsion, naked mRNAs, and dendritic cell-based delivery (each reviewed in Wang et al., supra). Decationic lipid nanoparticle (LNP) delivery is the most appealing and commonly used mRNA vaccine delivery tool.

Exogenous mRNA may be highly immunostimulatory. Single-stranded RNA (ssRNA) molecules are considered a pathogen associated molecular pattern (PAMP), and are recognised by various Toll-like receptors (TLR) which elicit a pro-inflammatory reaction. Although a strong cellular and humoral immune response is desirable in response to vaccination, the innate immune reaction elicited by exogenous mRNA may cause undesirable side-effects in the subject. The U-rich sequence of mRNA is a key element to activate TLR (Wang et al., supra). Additionally, enzymatically synthesised mRNA preparations contain double stranded RNA (dsRNA) contaminants as aberrant products of the in vitro transcription (IVT) process. dsRNA is a potent PAMP, and elicits downstream reactions resulting in the inhibition of translation and the degradation of cellular mRNA and ribosomal RNA (Pardi et al., supra). Thus, the mRNA may suppress antigen expression and thus reduce vaccine efficacy.

Studies over the past decade have shown that the immunostimulatory effect of mRNA can be shaped by the purification of IVT mRNA, the introduction of modified nucleosides, complexing the mRNA with various carrier molecules (Pardi et al., supra), adding poly(A) tails or optimising mRNA with GC-rich sequence (Wang et al., supra). Chemical modification of uridine is a common approach to minimise the immunogenicity of foreign mRNA. Incorporation of pseudouridine (ψ) and N1-methylpseudouridine (m1ψ) to IVT mRNA prevents TLR activation and other innate immune sensors, thus reducing pro-inflammatory signalling in response to the exogenous mRNA. Such nucleoside modification also suppresses recognition of dsRNA species (Pardi et al., supra) and can reduce innate immune sensing of exogenous mRNA translation (Hou et al. Nature Reviews Materials, 2021, https://doi.org/10.1038/s41578-021-00358-0).

Other nucleoside chemical modifications include, but are not limited to, 5-methylcytidine (m5C), 5-methyluridine (m5U), N1-methyladenosine (m1A), N6-methyladenosine (m6A), 2-thiouridine (s2U), and 5-methoxyuridine (5moU) (Wang et al., supra).

The IVT mRNA molecules used in the mRNA-1273 and BNT162b2 COVID-19 vaccines were prepared by replacing uridine with m1ψ, and their sequences were optimized to encode a stabilized pre-fusion spike protein with two pivotal proline substitutions (Hou et al., supra). However, CureVac's mRNA vaccine candidate, CVnCOV, uses unmodified nucleosides and relies on a combination of mRNA sequence alterations to allow immune evasion without affecting the expressed protein. Firstly, CVnCOV has a higher GC content (63%) than rival vaccines (BNT162b2 has 56%) and the original SARS-CoV-2 virus itself (37%). Secondly, the vaccine comprises C-rich motifs which bind to poly(C)-binding protein, enhancing both the stability and expression of the mRNA. A further modification of CVnCOV is that it contains a histone stem-loop sequence as well as a poly(A) tail, to enhance the longevity and translation of the mRNA (Hubert, B., 2021. The CureVac Vaccine, and a brief tour through some of the wonders of nature. URL https://berthub.eu/articles/posts/curevac-vaccine-and-wonders-of-biology/. (accessed 15.09.21). However, the vaccine had disappointing results from phase III clinical trials, which experts assert are down to the decision not to incorporate chemically modified nucleosides into the mRNA sequence. Nonetheless, CureVac and Acuitas Therapeutics delivered erythropoietin (EPO)-encoding mRNA, which has rich GC codons, to pigs with lipid nanoparticles (LNPs). Their results indicated EPO-related responses were elicited without immunogenicity (Wang et al., supra), suggesting that there is still scope for unmodified mRNA nucleoside-based vaccines.

A polynucleotide (or nucleic acid) of the invention may comprise an mRNA molecule.

The or each polynucleotide (or nucleic acid) of a pharmaceutical composition, a combined preparation, or a vector, of the invention may comprise an mRNA molecule.

A vector of the invention may be an mRNA vector.

The or each vector of a pharmaceutical composition or a combined preparation of the invention may be an mRNA vector.

A polynucleotide (or nucleic acid) of the invention, or a polynucleotide (or nucleic acid) of a pharmaceutical composition, a combined preparation, or a vector, of the invention, may be provided as part of an mRNA vaccine.

There is also provided according to the invention an mRNA vaccine which comprises a polynucleotide (or nucleic acid) of the invention, a vector of the invention, or a pharmaceutical composition or a combined preparation of the invention which comprises one or more polynucleotides (or nucleic acids), wherein the or each polynucleotide (or nucleic acid) comprises an mRNA molecule.

RNA or mRNA of a polynucleotide (or nucleic acid) of the invention, or of a polynucleotide (or nucleic acid) of a pharmaceutical composition, a combined preparation, a vector, or a vaccine, of the invention may be produced by in vitro transcription (IVT).

A polynucleotide (or nucleic acid) of the invention, or a polynucleotide (or nucleic acid) of a pharmaceutical composition, a combined preparation, a vector, or a vaccine, of the invention may comprise one or more modified nucleosides.

The one or more modified nucleosides may be present in DNA or RNA of a polynucleotide (or nucleic acid) of the invention, or of a polynucleotide (or nucleic acid) of a pharmaceutical composition, a combined preparation, a vector, or a vaccine, of the invention.

Optionally, at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, the chemical modification is in the 5-position of the uracil. In some embodiments, the chemical modification is a N1-methylpseudouridine. In some embodiments, the chemical modification is a N1-ethylpseudouridine.

For example, an RNA or an mRNA of a polynucleotide (or nucleic acid) of the invention, or of a polynucleotide (or nucleic acid) of a pharmaceutical composition, a combined preparation, a vector, or a vaccine, of the invention may comprise one or more of the following modified nucleosides:
pseudouridine (ψ);
N1-methylpseudouridine (m1ψ)
5-methylcytidine (m5C)
5-methyluridine (m5U)
N1-methyladenosine (m1A)
N6-methyladenosine (m6A)
2-thiouridine (s2U)
5-methoxyuridine (5moU)

In some embodiments, 100% of the uracil in the open reading frame have a chemical modification. In some embodiments, a chemical modification is in the 5-position of the uracil. In some embodiments, a chemical modification is a N1-methyl pseudouridine. In some embodiments, 100% of the uracil in the open reading frame have a N1-methyl pseudouridine in the 5-position of the uracil.

The polynucleotide (or nucleic acid) may contain from about 1% to about 100% modified nucleotides (or nucleosides) (either in relation to overall nucleotide content, or in relation to one or more types of nucleotide (or nucleoside), i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). Any remaining percentage is accounted for by the presence of unmodified A, G, U, or C.

Optionally a polynucleotide (or nucleic acid) of the invention, or of a polynucleotide (or nucleic acid) of a pharmaceutical composition, a combined preparation, a vector, or a vaccine, of the invention, comprises an RNA molecule in which the nucleic acid sequence of the polynucleotide (or nucleic acid) is the same as that recited in the respective SEQ ID, or the complement thereof, but with each ‘U’ replaced by m1ψ.

Optionally a polynucleotide (or nucleic acid) of the invention, or of a polynucleotide (or nucleic acid) of a pharmaceutical composition, a combined preparation, a vector, or a vaccine, of the invention, comprises an mRNA molecule in which the nucleic acid sequence of the polynucleotide is the same as that recited in the respective SEQ ID, or the complement thereof, but with each ‘U’ replaced by m1ψ.

Optionally a polynucleotide (or nucleic acid) of the invention, or of a polynucleotide (or nucleic acid) of a pharmaceutical composition, a combined preparation, a vector, or a vaccine, of the invention, comprises an RNA molecule in which the nucleic acid sequence of the polynucleotide (or nucleic acid) is the same as that recited in the respective SEQ ID, or the complement thereof, but with at least 50% of the ‘U’s replaced by m1ψ. The remaining ‘U’s may all be unmodified, or may comprise unmodified and one or more other modified nucleosides.

Optionally a polynucleotide (or nucleic acid) of the invention, or of a polynucleotide (or nucleic acid) of a pharmaceutical composition, a combined preparation, a vector, or a vaccine, of the invention, comprises an mRNA molecule in which the nucleic acid sequence of the polynucleotide (or nucleic acid) is the same as that recited in the respective SEQ ID, or the complement thereof, but with at least 50% of the ‘U’s replaced by m1ψ. The remaining ‘U’s may all be unmodified, or may comprise unmodified and one or more other modified nucleosides.

Optionally a polynucleotide (or nucleic acid) of the invention, or of a polynucleotide (or nucleic acid) of a pharmaceutical composition, a combined preparation, a vector, or a vaccine, of the invention, comprises an RNA molecule in which the nucleic acid sequence of the polynucleotide (or nucleic acid) is the same as that recited in the respective SEQ ID, or the complement thereof, but with at least 90% of the ‘U’s replaced by m1ψ. The remaining ‘U’s may all be unmodified, or may comprise unmodified and one or more other modified nucleosides.

Optionally a polynucleotide (or nucleic acid) of the invention, or of a polynucleotide (or nucleic acid) of a pharmaceutical composition, a combined preparation, a vector, or a vaccine, of the invention, comprises an mRNA molecule in which the nucleic acid sequence of the polynucleotide (or nucleic acid) is the same as that recited in the respective SEQ ID, or the complement thereof, but with at least 90% of the ‘U’s replaced by m1ψ. The remaining ‘U’s may all be unmodified, or may comprise unmodified and one or more other modified nucleosides.

mRNA vaccines of the invention may be co-administered with an immunological adjuvant, for example MF59 (Novartis), TriMix, RNActive (CureVac AG), RNAdjuvant (again reviewed in Wang et al., supra).

Where mRNA vaccines encoding different polypeptides of the invention are used in accordance with the invention, it is preferred that each different polypeptide of the invention (for example, a designed coronavirus S protein (full length, truncated, or RBD) of the invention and/or a designed coronavirus E protein of the invention and/or a designed coronavirus M protein of the invention and/or a designed coronavirus N protein of the invention), is encoded as part of a separate mRNA vaccine vector.

Thus, in preferred embodiments, each vector of a pharmaceutical composition, or combined preparation, of the invention is an mRNA vaccine vector.

There is also provided according to the invention an isolated cell comprising or transfected with a vector of the invention.

There is also provided according to the invention a fusion protein comprising a polypeptide of the invention.

### Pharmaceutical Compositions

According to the invention there is also provided a pharmaceutical composition comprising a polypeptide of the invention, and a pharmaceutically acceptable carrier, excipient, or diluent.

Optionally a pharmaceutical composition of the invention comprises more than one different polypeptide of the invention.

Advantageously, a pharmaceutical composition of the invention comprises a designed coronavirus S protein (full length, truncated, or RBD) of the invention and/or a designed coronavirus E protein of the invention and/or a designed coronavirus M protein of the invention.

Advantageously, a pharmaceutical composition of the invention comprises a designed coronavirus S protein (full length, truncated, or RBD) of the invention and/or a designed coronavirus E protein of the invention and/or a designed coronavirus M protein of the invention and/or a designed coronavirus N protein of the invention.

Optionally a pharmaceutical composition of the invention comprises a designed coronavirus S protein (full length, truncated, or RBD) of the invention and a designed coronavirus E protein of the invention.

Optionally a pharmaceutical composition of the invention comprises a designed coronavirus S protein (full length, truncated, or RBD) of the invention and a designed coronavirus M protein of the invention.

Optionally a pharmaceutical composition of the invention comprises a designed coronavirus S protein (full length, truncated, or RBD) of the invention and a designed coronavirus N protein of the invention.

Optionally a pharmaceutical composition of the invention comprises a designed coronavirus E protein of the invention and a designed coronavirus M protein of the invention.

Optionally a pharmaceutical composition of the invention comprises a designed coronavirus E protein of the invention and a designed coronavirus N protein of the invention.

Optionally a pharmaceutical composition of the invention comprises a designed coronavirus S protein (full length, truncated, or RBD) of the invention and a designed coronavirus E protein of the invention and a designed coronavirus M protein of the invention.

Optionally a pharmaceutical composition of the invention comprises a designed coronavirus S protein (full length, truncated, or RBD) of the invention and a designed coronavirus E protein of the invention and a designed coronavirus N protein of the invention.

Optionally a pharmaceutical composition of the invention comprises a designed coronavirus E protein of the invention and a designed coronavirus M protein of the invention and a designed coronavirus N protein of the invention.

Optionally a pharmaceutical composition of the invention comprises:
a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17; and
a polypeptide of the invention which comprises an amino acid sequence of SEQ ID NO: 22, or an amino acid sequence which has at least 95%, 96%, 97%, 98%, or 99% amino acid identity over its entire length with the amino acid sequence of SEQ ID NO:22, or a polypeptide of the invention which comprises an amino acid sequence of SEQ ID NO:23, or an amino acid sequence which has at least 98%, or 99% amino acid identity over its entire length with the amino acid sequence of SEQ ID NO:23.

Optionally a pharmaceutical composition of the invention comprises:
a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17; and
a polypeptide of the invention which comprises an amino acid sequence of SEQ ID NO: 24, or an amino acid sequence which has at least 91%, 92%, 93%, 94%, 95%, 96%, 97%, 98%, or 99% amino acid identity over its entire length with the amino acid sequence of SEQ ID NO: 24, or a polypeptide of the invention which comprises an amino acid sequence of SEQ ID NO: 25, or an amino acid sequence which has at least 95%, 96%, 97%, 98%, or 99% amino acid identity over its entire length with the amino acid sequence of SEQ ID NO:25.

Optionally a pharmaceutical composition of the invention comprises:
a polypeptide of the invention which comprises an amino acid sequence of SEQ ID NO: 22, or an amino acid sequence which has at least 95%, 96%, 97%, 98%, or 99% amino acid identity over its entire length with the amino acid sequence of SEQ ID NO:22, or a polypeptide of the invention which comprises an amino acid sequence of SEQ ID NO:23, or an amino acid sequence which has at least 98%, or 99% amino acid identity over its entire length with the amino acid sequence of SEQ ID NO:23; and
a polypeptide of the invention which comprises an amino acid sequence of SEQ ID NO: 24, or an amino acid sequence which has at least 91%, 92%, 93%, 94%, 95%, 96%, 97%, 98%, or 99% amino acid identity over its entire length with the amino acid sequence of SEQ ID NO: 24, or a polypeptide of the invention which comprises an amino acid sequence of SEQ ID NO: 25, or an amino acid sequence which has at least 95%, 96%, 97%, 98%, or 99% amino acid identity over its entire length with the amino acid sequence of SEQ ID NO:25.

Optionally a pharmaceutical composition of the invention comprises:
a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17; and
a polypeptide of the invention which comprises an amino acid sequence of SEQ ID NO: 22, or an amino acid sequence which has at least 95%, 96%, 97%, 98%, or 99% amino acid identity over its entire length with the amino acid sequence of SEQ ID NO:22, or a polypeptide of the invention which comprises an amino acid sequence of SEQ ID NO:23, or an amino acid sequence which has at least 98%, or 99% amino acid identity over its entire length with the amino acid sequence of SEQ ID NO:23; and
a polypeptide of the invention which comprises an amino acid sequence of SEQ ID NO: 24, or an amino acid sequence which has at least 91%, 92%, 93%, 94%, 95%, 96%, 97%, 98%, or 99% amino acid identity over its entire length with the amino acid sequence of SEQ ID NO: 24, or a polypeptide of the invention which comprises an amino acid sequence of SEQ ID NO: 25, or an amino acid sequence which has at least 95%, 96%, 97%, 98%, or 99% amino acid identity over its entire length with the amino acid sequence of SEQ ID NO:25.

According to the invention there is also provided a pharmaceutical composition comprising a nucleic acid of the invention, and a pharmaceutically acceptable carrier, excipient, or diluent.

Optionally a pharmaceutical composition of the invention comprises more than one nucleic acid molecule of the invention encoding a different polypeptide of the invention.

Advantageously, a pharmaceutical composition of the invention comprises a nucleic acid molecule of the invention encoding a designed coronavirus S protein (full length, truncated, or RBD) of the invention and/or a nucleic acid molecule of the invention encoding a designed coronavirus E protein of the invention and/or a nucleic acid molecule of the invention encoding a designed coronavirus M protein of the invention.

Advantageously, a pharmaceutical composition of the invention comprises a nucleic acid molecule of the invention encoding a designed coronavirus S protein (full length, truncated, or RBD) of the invention and/or a nucleic acid molecule of the invention encoding a designed coronavirus E protein of the invention and/or a nucleic acid molecule of the invention encoding a designed coronavirus M protein of the invention and/or a nucleic acid molecule of the invention encoding a designed coronavirus N protein of the invention.

Optionally a pharmaceutical composition of the invention comprises a nucleic acid molecule of the invention encoding a designed coronavirus S protein (full length, truncated, or RBD) of the invention and a nucleic acid molecule of the invention encoding a designed coronavirus E protein of the invention.

Optionally a pharmaceutical composition of the invention comprises a nucleic acid molecule of the invention encoding a designed coronavirus S protein (full length, truncated, or RBD) of the invention and a nucleic acid molecule of the invention encoding a designed coronavirus M protein of the invention.

Optionally a pharmaceutical composition of the invention comprises a nucleic acid molecule of the invention encoding a designed coronavirus S protein (full length, truncated, or RBD) of the invention and a nucleic acid molecule of the invention encoding a designed coronavirus N protein of the invention.

Optionally a pharmaceutical composition of the invention comprises a nucleic acid molecule of the invention encoding a designed coronavirus E protein of the invention and a nucleic acid molecule of the invention encoding a designed coronavirus M protein of the invention.

Optionally a pharmaceutical composition of the invention comprises a nucleic acid molecule of the invention encoding a designed coronavirus E protein of the invention and a nucleic acid molecule of the invention encoding a designed coronavirus N protein of the invention.

Optionally a pharmaceutical composition of the invention comprises a nucleic acid molecule of the invention encoding a designed coronavirus S protein (full length, truncated, or RBD) of the invention and a nucleic acid molecule of the invention encoding a designed coronavirus E protein of the invention and a nucleic acid molecule of the invention encoding a designed coronavirus M protein of the invention.

Optionally a pharmaceutical composition of the invention comprises a nucleic acid molecule of the invention encoding a designed coronavirus S protein (full length, truncated, or RBD) of the invention and a nucleic acid molecule of the invention encoding a designed coronavirus E protein of the invention and a nucleic acid molecule of the invention encoding a designed coronavirus N protein of the invention.

Optionally a pharmaceutical composition of the invention comprises a nucleic acid molecule of the invention encoding a designed coronavirus E protein of the invention and a nucleic acid molecule of the invention encoding a designed coronavirus M protein of the invention and a nucleic acid molecule of the invention encoding a designed coronavirus N protein of the invention.

Optionally a pharmaceutical composition of the invention comprises:
a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17; and
a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23.

Optionally a pharmaceutical composition of the invention comprises:
a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17; and
a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally a pharmaceutical composition of the invention comprises:
a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23; and
a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally a pharmaceutical composition of the invention comprises:
a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17; and
a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23; and
a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

According to the invention there is also provided a pharmaceutical composition comprising a vector of the invention, and a pharmaceutically acceptable carrier, excipient, or diluent.

Optionally a pharmaceutical composition of the invention further comprises an adjuvant for enhancing an immune response in a subject to the polypeptide, or to a polypeptide encoded by the nucleic acid, of the composition.

Optionally a pharmaceutical composition of the invention further comprises an adjuvant for enhancing an immune response in a subject to the polypeptides, or to polypeptides encoded by the nucleic acids, of the composition.

There is also provided according to the invention a pseudotyped virus comprising a polypeptide of the invention.

### Combined Preparations

The term “combined preparation” as used herein refers to a “kit of parts” in the sense that the combination components (i) and (ii), or (i), (ii) and (iii), or (i), (ii) (iii) and (iv) as defined herein, can be dosed independently or by use of different fixed combinations with distinguished amounts of the combination components (i) and (ii), or (i), (ii) and (iii), or (i), (ii) (iii) and (iv). The components can be administered simultaneously or one after the other. If the components are administered one after the other, preferably the time interval between administration is chosen such that the therapeutic effect of the combined use of the components is greater than the effect which would be obtained by use of only any one of the combination components (i) and (ii), or (i), (ii) and (iii), or (i), (ii) (iii) and (iv).

The components of the combined preparation may be present in one combined unit dosage form, or as a first unit dosage form of component (i) and a separate, second unit dosage form of component (ii), or as a first unit dosage form of component (i), a separate, second unit dosage form of component (ii), and a separate, third unit dosage form of component (iii), or as a first unit dosage form of component (i), a separate, second unit dosage form of component (ii), a separate, third unit dosage form of component (iii), and a separate, third unit dosage form of component (iv). The ratio of the total amounts of the combination component (i) to the combination component (ii), or of the combination component (i) to the combination component (ii) and to the combination component (iii), or of the combination component (i) to the combination component (ii) to the combination component (iii) and to the combination component (iv) to be administered in the combined preparation can be varied, for example in order to cope with the needs of a patient sub-population to be treated, or the needs of the single patient, which can be due, for example, to the particular disease, age, sex, or body weight of the patient.

Preferably, there is at least one beneficial effect, for example an enhancing of the effect of the component (i), or an enhancing of the effect of the component (ii), or a mutual enhancing of the effect of the combination components (i) and (ii), or an enhancing of the effect of the component (i), or an enhancing of the effect of the component (ii), or an enhancing of the effect of the component (iii), or a mutual enhancing of the effect of the combination components (i), (ii), and (iii), or an enhancing of the effect of the component (i), or an enhancing of the effect of the component (ii), or an enhancing of the effect of the component (iii), or an enhancing of the effect of the component (iv), or a mutual enhancing of the effect of the combination components (i), (ii), (iii), and (iv), for example a more than additive effect, additional advantageous effects, fewer side effects, less toxicity, or a combined therapeutic effect compared with an effective dosage of one or both of the combination components (i) and (ii), or (i), (ii), and (iii), or (i), (ii), (iii), and (iv), and very preferably a synergism of the combination components (i) and (ii), or (i), (ii), and (iii), or (i), (ii), (iii), and (iv).

A combined preparation of the invention may be provided as a pharmaceutical combined preparation for administration to a mammal, preferably a human. The component (i) may optionally be provided together with a pharmaceutically acceptable carrier, excipient, or diluent, and/or the component (ii) may optionally be provided together with a pharmaceutically acceptable carrier, excipient, or diluent, or the component (i) may optionally be provided together with a pharmaceutically acceptable carrier, excipient, or diluent, and/or the component (ii) may optionally be provided together with a pharmaceutically acceptable carrier, excipient, or diluent and/or the component (iii) may optionally be provided together with a pharmaceutically acceptable carrier, excipient, or diluent, or the component (i) may optionally be provided together with a pharmaceutically acceptable carrier, excipient, or diluent, and/or the component (ii) may optionally be provided together with a pharmaceutically acceptable carrier, excipient, or diluent and/or the component (iii) may optionally be provided together with a pharmaceutically acceptable carrier, excipient, or diluent and/or the component (iv) may optionally be provided together with a pharmaceutically acceptable carrier, excipient, or diluent.

According to the invention there is provided a combined preparation, which comprises:
i) a designed coronavirus S protein (full length, truncated, or RBD) of the invention; and/or
ii) a designed coronavirus E protein of the invention; and/or
iii) a designed coronavirus M protein of the invention; and/or
iv) a designed coronavirus N protein of the invention.

According to the invention there is provided a combined preparation, which comprises:
i) a designed coronavirus S protein (full length, truncated, or RBD) of the invention; and/or
ii) a designed coronavirus E protein of the invention; and/or
iii) a designed coronavirus M protein of the invention.

According to the invention there is provided a combined preparation, which comprises:
i) a designed coronavirus S protein (full length, truncated, or RBD) of the invention;
ii) a designed coronavirus E protein of the invention;
iii) a designed coronavirus M protein of the invention; and
iv) a designed coronavirus N protein of the invention.

According to the invention there is provided a combined preparation, which comprises:
i) a designed coronavirus S protein (full length, truncated, or RBD) of the invention; and
ii) a designed coronavirus E protein of the invention.

According to the invention there is provided a combined preparation, which comprises:
i) a designed coronavirus S protein (full length, truncated, or RBD) of the invention; and
ii) a designed coronavirus M protein of the invention.

According to the invention there is provided a combined preparation, which comprises:
i) a designed coronavirus S protein (full length, truncated, or RBD) of the invention; and
ii) a designed coronavirus N protein of the invention.

According to the invention there is provided a combined preparation, which comprises:
i) a designed coronavirus E protein of the invention; and
ii) a designed coronavirus M protein of the invention.

According to the invention there is provided a combined preparation, which comprises:
i) a designed coronavirus E protein of the invention; and
ii) a designed coronavirus N protein of the invention.

According to the invention there is provided a combined preparation, which comprises:
i) a designed coronavirus S protein (full length, truncated, or RBD) of the invention; and
ii) a designed coronavirus E protein of the invention; and
iii) a designed coronavirus M protein of the invention.

According to the invention there is provided a combined preparation, which comprises:
i) a designed coronavirus S protein (full length, truncated, or RBD) of the invention; and
ii) a designed coronavirus E protein of the invention; and
iii) a designed coronavirus N protein of the invention.

According to the invention there is provided a combined preparation, which comprises:
i) a designed coronavirus E protein of the invention; and
ii) a designed coronavirus M protein of the invention; and
iii) a designed coronavirus N protein of the invention.

Optionally a combined preparation of the invention comprises:
i) a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17; and
ii) a polypeptide of the invention which comprises an amino acid sequence of SEQ ID NO: 22, or an amino acid sequence which has at least 95%, 96%, 97%, 98%, or 99% amino acid identity over its entire length with the amino acid sequence of SEQ ID NO:22, or a polypeptide of the invention which comprises an amino acid sequence of SEQ ID NO:23, or an amino acid sequence which has at least 98%, or 99% amino acid identity over its entire length with the amino acid sequence of SEQ ID NO:23.

Optionally a combined preparation of the invention comprises:
i) a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17; and
ii) a polypeptide of the invention which comprises an amino acid sequence of SEQ ID NO: 24, or an amino acid sequence which has at least 91%, 92%, 93%, 94%, 95%, 96%, 97%, 98%, or 99% amino acid identity over its entire length with the amino acid sequence of SEQ ID NO: 24, or a polypeptide of the invention which comprises an amino acid sequence of SEQ ID NO: 25, or an amino acid sequence which has at least 95%, 96%, 97%, 98%, or 99% amino acid identity over its entire length with the amino acid sequence of SEQ ID NO:25.

Optionally a combined preparation of the invention comprises:
i) a polypeptide of the invention which comprises an amino acid sequence of SEQ ID NO: 22, or an amino acid sequence which has at least 95%, 96%, 97%, 98%, or 99% amino acid identity over its entire length with the amino acid sequence of SEQ ID NO:22, or a polypeptide of the invention which comprises an amino acid sequence of SEQ ID NO:23, or an amino acid sequence which has at least 98%, or 99% amino acid identity over its entire length with the amino acid sequence of SEQ ID NO:23; and
ii) a polypeptide of the invention which comprises an amino acid sequence of SEQ ID NO: 24, or an amino acid sequence which has at least 91%, 92%, 93%, 94%, 95%, 96%, 97%, 98%, or 99% amino acid identity over its entire length with the amino acid sequence of SEQ ID NO: 24, or a polypeptide of the invention which comprises an amino acid sequence of SEQ ID NO: 25, or an amino acid sequence which has at least 95%, 96%, 97%, 98%, or 99% amino acid identity over its entire length with the amino acid sequence of SEQ ID NO:25.

Optionally a combined preparation of the invention comprises:
i) a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17; and
ii) a polypeptide of the invention which comprises an amino acid sequence of SEQ ID NO: 22, or an amino acid sequence which has at least 95%, 96%, 97%, 98%, or 99% amino acid identity over its entire length with the amino acid sequence of SEQ ID NO:22, or a polypeptide of the invention which comprises an amino acid sequence of SEQ ID NO:23, or an amino acid sequence which has at least 98%, or 99% amino acid identity over its entire length with the amino acid sequence of SEQ ID NO:23; and
iii) a polypeptide of the invention which comprises an amino acid sequence of SEQ ID NO: 24, or an amino acid sequence which has at least 91%, 92%, 93%, 94%, 95%, 96%, 97%, 98%, or 99% amino acid identity over its entire length with the amino acid sequence of SEQ ID NO: 24, or a polypeptide of the invention which comprises an amino acid sequence of SEQ ID NO: 25, or an amino acid sequence which has at least 95%, 96%, 97%, 98%, or 99% amino acid identity over its entire length with the amino acid sequence of SEQ ID NO:25.

According to the invention there is provided a combined preparation, which comprises:
i) a nucleic acid molecule of the invention encoding a designed coronavirus S protein (full length, truncated, or RBD) of the invention; and/or
ii) a nucleic acid molecule of the invention encoding a designed coronavirus E protein of the invention; and/or
iii) a nucleic acid molecule of the invention encoding a designed coronavirus M protein of the invention.

According to the invention there is provided a combined preparation, which comprises:
i) a nucleic acid molecule of the invention encoding a designed coronavirus S protein (full length, truncated, or RBD) of the invention; and/or
ii) a nucleic acid molecule of the invention encoding a designed coronavirus E protein of the invention; and/or
iii) a nucleic acid molecule of the invention encoding a designed coronavirus M protein of the invention; and/or
iv) a nucleic acid molecule of the invention encoding a designed coronavirus N protein of the invention.

According to the invention there is provided a combined preparation, which comprises:
i) a nucleic acid molecule of the invention encoding a designed coronavirus S protein (full length, truncated, or RBD) of the invention;
ii) a nucleic acid molecule of the invention encoding a designed coronavirus E protein of the invention;
iii) a nucleic acid molecule of the invention encoding a designed coronavirus M protein of the invention; and
iv) a nucleic acid molecule of the invention encoding a designed coronavirus N protein of the invention.

According to the invention there is provided a combined preparation, which comprises:
i) a nucleic acid molecule of the invention encoding a designed coronavirus S protein (full length, truncated, or RBD) of the invention; and
ii) a nucleic acid molecule of the invention encoding a designed coronavirus E protein of the invention.

According to the invention there is provided a combined preparation, which comprises:
i) a nucleic acid molecule of the invention encoding a designed coronavirus S protein (full length, truncated, or RBD) of the invention; and
ii) a nucleic acid molecule of the invention encoding a designed coronavirus M protein of the invention.

According to the invention there is provided a combined preparation, which comprises:
i) a nucleic acid molecule of the invention encoding a designed coronavirus S protein (full length, truncated, or RBD) of the invention; and
ii) a nucleic acid molecule of the invention encoding a designed coronavirus N protein of the invention.

According to the invention there is provided a combined preparation, which comprises:
i) a nucleic acid molecule of the invention encoding a designed coronavirus E protein of the invention; and
ii) a nucleic acid molecule of the invention encoding a designed coronavirus M protein of the invention.

According to the invention there is provided a combined preparation, which comprises:
i) a nucleic acid molecule of the invention encoding a designed coronavirus E protein of the invention; and
ii) a nucleic acid molecule of the invention encoding a designed coronavirus N protein of the invention.

According to the invention there is provided a combined preparation, which comprises:
i) a nucleic acid molecule of the invention encoding a designed coronavirus S protein (full length, truncated, or RBD) of the invention; and
ii) a nucleic acid molecule of the invention encoding a designed coronavirus E protein of the invention; and
iii) a nucleic acid molecule of the invention encoding a designed coronavirus M protein of the invention.

According to the invention there is provided a combined preparation, which comprises:
i) a nucleic acid molecule of the invention encoding a designed coronavirus S protein (full length, truncated, or RBD) of the invention; and
ii) a nucleic acid molecule of the invention encoding a designed coronavirus E protein of the invention; and
iii) a nucleic acid molecule of the invention encoding a designed coronavirus N protein of the invention.

According to the invention there is provided a combined preparation, which comprises:
i) a nucleic acid molecule of the invention encoding a designed coronavirus E protein of the invention; and
ii) a nucleic acid molecule of the invention encoding a designed coronavirus M protein of the invention; and
iii) a nucleic acid molecule of the invention encoding a designed coronavirus N protein of the invention.

Optionally a combined preparation of the invention comprises:
i) a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17; and
ii) a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23.

Optionally a combined preparation of the invention comprises:
i) a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17; and
ii) a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally a combined preparation of the invention comprises:
i) a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23; and
ii) a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally a combined preparation of the invention comprises:
i) a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17; and
ii) a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23; and
iii) a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Each different nucleic acid molecule of a combined preparation of the invention may be provided as part of a separate vector.

According to the invention there is also provided a combined preparation comprising a vector of the invention, and a pharmaceutically acceptable carrier, excipient, or diluent.

Optionally a combined preparation of the invention further comprises an adjuvant for enhancing an immune response in a subject to the polypeptide, or to a polypeptide encoded by the nucleic acid, of the composition.

Optionally a combined preparation of the invention further comprises an adjuvant for enhancing an immune response in a subject to the polypeptides, or to polypeptides encoded by the nucleic acids, of the composition.

### Strings

Embodiments of the invention in which different polypeptides of the invention are encoded as part of the same polynucleotide (or nucleic acid), or are provided in the same polypeptide (i.e. as “strings” of different subunits, e.g. S protein RBD and/or E protein and/or M protein, and/or N protein), are particularly advantageous since use of such a “string” as part of a vaccine requires testing only of the single product containing the “string” for safety and efficacy, rather than testing each different subunit individually. This dramatically reduces the time and cost of developing the vaccine compared with individual subunits. In some embodiments, a combination of different strings (polynucleotide and/or polypeptide), or a combination of one or more strings and one or more single subunits (polypeptide or encoded subunit) may be used.

Strategies for multigene co-expression include introduction of multiple vectors, use of multiple promoters in a single vector, fusion proteins, proteolytic cleavage sites between genes, internal ribosome entry sites (IRES), and “self-cleaving” 2A peptides. Multicistronic vectors based on IRES nucleotide sequence and self-cleaving 2A peptides are reviewed in Shaimardanova et al. (Pharmaceutics 2019, 11, 580; doi: 10.3390/pharmaceutics11110580).

### Vaccines

Vaccines may be provided, for example, as nucleic acid vaccines, either as separate polynucleotides, each encoding a different subunit (for administration together or separately) or pieced together in a string as a single polynucleotide encoding all of the subunits. The separate polynucleotides may be administered as a mixture together (for example, as a pharmaceutical composition comprising the separate polynucleotides), or co-administered or administered sequentially in any order (in which case, the separate polynucleotides may be provided as a combined preparation for co-administration or sequential administration). Nucleic acid vaccines may be provided as DNA, RNA, or mRNA vaccines. Production and application of multicistronic constructs (for example, where the subunits are provided in a string as a single polynucleotide) is reviewed by Shaimardanova et al. (Pharmaceutics 2019, 11, 580; doi: 10.3390/pharmaceutics11110580).

Vaccine constructs of the invention may also be provided, for example, either as separate polypeptides, each comprising a different designed subunit or pieced together in a string as a single polypeptide comprising all of the subunits. The separate polypeptides may be administered as a mixture together (for example, as a pharmaceutical composition comprising the separate polypeptides), or co-administered or administered sequentially in any order (in which case, the separate polypeptides may be provided as a combined preparation for co-administration or sequential administration).

### Methods of Treatment and Uses

There is also provided according to the invention a method of inducing an immune response to a coronavirus in a subject, which comprises administering to the subject an effective amount of a polypeptide of the invention, a nucleic acid of the invention, a vector of the invention, or a pharmaceutical composition of the invention.

There is also provided according to the invention a method of immunising a subject against a coronavirus, which comprises administering to the subject an effective amount of a polypeptide of the invention, a nucleic acid of the invention, a vector of the invention, or a pharmaceutical composition of the invention.

An effective amount is an amount to produce an antigen-specific immune response in a subject.

There is further provided according to the invention a polypeptide of the invention, a nucleic acid of the invention, a vector of the invention, or a pharmaceutical composition of the invention, for use as a medicament.

There is further provided according to the invention a polypeptide of the invention, a nucleic acid of the invention, a vector of the invention, or a pharmaceutical composition of the invention, for use in the prevention, treatment, or amelioration of a coronavirus infection.

There is also provided according to the invention use of a polypeptide of the invention, a nucleic acid of the invention, a vector of the invention, or a pharmaceutical composition of the invention, in the manufacture of a medicament for the prevention, treatment, or amelioration of a coronavirus infection.

Optionally the coronavirus is a β-coronavirus.

Optionally the β-coronavirus is a lineage B or C β-coronavirus.

Optionally the β-coronavirus is a lineage B β-coronavirus.

Optionally the lineage B β-coronavirus is SARS-CoV or SARS-CoV-2.

Optionally the lineage C β-coronavirus is MERS-COV.

Optionally an immune response is induced against more than one lineage B beta-coronavirus.

Optionally an immune response is induced against SARS-1 and SARS-2 beta-coronavirus.

Optionally an immune response is induced against SARS-1 and MERS beta-coronavirus.

Optionally an immune response is induced against SARS-2 and MERS beta-coronavirus.

Optionally an immune response is induced against SARS-1, SARS-2, and MERS beta-coronavirus.

Optionally the beta-coronavirus is a variant of concern (VOC).

Optionally the beta-coronavirus is a SARS-CoV-2 VOC.

Optionally the beta-coronavirus is a SARS-CoV-2 lineage B1.248 (Brazil P1 lineage) VOC.

Optionally the beta-coronavirus is a SARS-CoV-2 lineage B1.351 (South Africa) VOC.

Optionally the beta-coronavirus is a SARS-CoV-2 beta, gamma, or delta VOC.

Optionally the beta-coronavirus is a SARS-CoV-2 beta VOC.

Optionally the beta-coronavirus is a SARS-CoV-2 gamma VOC.

Optionally the beta-coronavirus is a SARS-CoV-2 delta VOC.

Optionally the beta-coronavirus is a SARS-CoV-2 alpha VOC.

Optionally the beta-coronavirus is a SARS-CoV-2 omicron VOC.

Optionally the beta-coronavirus is SARS-CoV-2 omicron BA. 1.

Optionally the beta-coronavirus is a SARS-CoV-2 omicron BA.2.

It can readily be determined whether an immune response has been induced to a beta-coronavirus using methods well-known to the skilled person. For example, a pseudotype neutralisation assay as described in any of the examples below may be used.

### Administration

Any suitable route of administration may be used. Methods of administration include, but are not limited to, intradermal, intramuscular, intraperitoneal, parenteral, intravenous, subcutaneous, vaginal, rectal, intranasal, inhalation or oral. Parenteral administration, such as subcutaneous, intravenous or intramuscular administration, is generally achieved by injection. Injectables can be prepared in conventional forms, either as liquid solutions or suspensions, solid forms suitable for solution or suspension in liquid prior to injection, or as emulsions. Injection solutions and suspensions can be prepared from sterile powders, granules, and tablets of the kind previously described. Administration can be systemic or local. Routes for systemic administration in general include, for example, transdermal, oral, parenteral routes, including subcutaneous, intravenous, intramuscular, intraarterial, intradermal and intraperitoneal injections and/or intranasal administration routes. Routes for local administration in general include, for example, topical administration routes but also intradermal, transdermal, subcutaneous, or intramuscular injections or intralesional, intracranial, intrapulmonal, intracardial, and sublingual injections.

Compositions may be administered in any suitable manner, such as with pharmaceutically acceptable carriers. Pharmaceutically acceptable carriers are determined in part by the particular composition being administered, as well as by the particular method used to administer the composition. Preparations for parenteral administration include sterile aqueous or nonaqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Administration can be accomplished by single or multiple doses. The dose administered to a subject in the context of the present disclosure should be sufficient to induce a beneficial therapeutic response in a subject over time, or to inhibit or prevent infection. The dose required will vary from subject to subject depending on the species, age, weight and general condition of the subject, the severity of the infection being treated, the particular composition being used and its mode of administration. An appropriate dose can be determined by one of ordinary skill in the art using only routine experimentation.

The present disclosure includes methods comprising administering an RNA vaccine, an mRNA vaccine, or a DNA vaccine to a subject in need thereof. The exact amount required will vary from subject to subject, depending on the species, age, and general condition of the subject, the severity of the disease, the particular composition, its mode of administration, its mode of activity, and the like.

The RNA or DNA is typically formulated in dosage unit form for ease of administration and uniformity of dosage. It will be understood, however, that the total daily usage of the RNA may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount of the RNA or DNA, as provided herein, may be as low as 20 pg, administered for example as a single dose or as two 10 pg doses. In some embodiments, the effective amount is a total dose of 20 μg-300 μg or 25 μg-300 μg. For example, the effective amount may be a total dose of 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 250 μg, or 300 μg. In some embodiments, the effective amount is a total dose of 20 μg. In some embodiments, the effective amount is a total dose of 25 pg. In some embodiments, the effective amount is a total dose of 50 μg. In some embodiments, the effective amount is a total dose of 75 μg. In some embodiments, the effective amount is a total dose of 100 μg. In some embodiments, the effective amount is a total dose of 150 μg. In some embodiments, the effective amount is a total dose of 200 μg. In some embodiments, the effective amount is a total dose of 250 pg. In some embodiments, the effective amount is a total dose of 300 μg.

The RNA or DNA described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

Optionally, an RNA (e.g., mRNA) or DNA vaccine is formulated in an effective amount to produce an antigen specific immune response in a subject.

In some embodiments, the effective amount is a total dose of 25 μg to 1000 μg, or 50 μg to 1000 μg. In some embodiments, the effective amount is a total dose of 100 μg. In some embodiments, the effective amount is a dose of 25 μg administered to the subject a total of two times. In some embodiments, the effective amount is a dose of 100 μg administered to the subject a total of two times. In some embodiments, the effective amount is a dose of 400 μg administered to the subject a total of two times. In some embodiments, the effective amount is a dose of 500 μg administered to the subject a total of two times.

Optionally a dosage of between 10 μg/kg and 400 μg/kg of the nucleic acid vaccine is administered to the subject. In some embodiments the dosage of the RNA or DNA polynucleotide (or nucleic acid) is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the nucleic acid vaccine is administered to the subject by intradermal or intramuscular injection. In some embodiments, the nucleic acid vaccine is administered to the subject on day zero. In some embodiments, a second dose of the nucleic acid vaccine is administered to the subject on day twenty one.

### Pharmaceutically Acceptable Carriers

Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, and magnesium carbonate. Any of the common pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used with the compositions and methods provided herein are normal saline and sesame oil.

In some embodiments, the compositions comprise a pharmaceutically acceptable carrier and/or an adjuvant. For example, the adjuvant can be alum, Freund's complete adjuvant, a biological adjuvant or immunostimulatory oligonucleotides (such as CpG oligonucleotides).

The pharmaceutically acceptable carriers (vehicles) useful in this disclosure are conventional. Remington's Pharmaceutical Sciences, by E. W. Martin, Mack Publishing Co., Easton, PA, 15th Edition (1975), describes compositions and formulations suitable for pharmaceutical delivery of one or more therapeutic compositions, such as one or more influenza vaccines, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Optionally a polypeptide, nucleic acid, or composition of the invention is administered intramuscularly.

Optionally a polypeptide, nucleic acid, or composition of the invention is administered intramuscularly, intradermally, subcutaneously by needle or by gene gun, or electroporation.

### Diagnostic Methods

There is also provided according to the invention a method of diagnosing whether a subject has a coronavirus infection, which comprises determining whether a polypeptide of the invention is bound by antibodies produced by the subject.

Optionally the method is an in vitro method.

Optionally the antibodies are in a biological sample obtained from the subject, or in a sample derived from a biological sample obtained from the subject.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples, and cellular samples.

Optionally the biological sample is selected from the group consisting of blood, serum, plasma, urine, saliva, cerebrospinal fluid, interstitial fluid, ocular fluid, synovial fluid, solid tissue sample, tissue culture sample, and cellular sample.

Optionally the biological sample is a blood or a serum sample.

Suitable methods for determining whether a polypeptide of the invention is bound by antibodies produced by the subject are well-known to those skilled in the art, including, for example, ELISA, luminex, legendplex.

A diagnostic method of the present invention can be used to determine the stage (severity) of a coronavirus infection. A diagnostic method of the present invention can be used to monitor progression of a coronavirus infection in the subject. A diagnostic method of the invention can be used to determine a subject's response to a treatment regimen for treating a coronavirus infection.

Diagnostic methods of the invention generally involve (a) determining the amount of an antibody (or antibodies) bound by a polypeptide of the invention in a biological sample obtained from the subject; and (b) comparing the amount of the antibody (or antibodies) in the biological sample to a reference, a standard, or a normal control value that indicates the amount of the antibody (or antibodies) in normal control subjects. A significant difference between the amount of antibody (or antibodies) in the biological sample and the normal control value indicates that the individual has a coronavirus infection. In some embodiments, the step of determining comprises contacting the biological sample with a polypeptide of the invention and quantitating binding of the polypeptide to the antibody (or antibodies) present in the sample.

Various aspects of the invention are defined in the following numbered paragraphs:

1. An isolated polypeptide which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 17.

2. An isolated polypeptide comprising an amino acid sequence of SEQ ID NO: 15, or an amino acid sequence which has at least 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 15.

3. An isolated polypeptide comprising an amino acid sequence of SEQ ID NO: 13, or an amino acid sequence which has at least 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 13.

4. An isolated polypeptide according to any preceding paragraph, which comprises at least one of the amino acid residues, at a position corresponding to the amino acid residue position of SEQ ID NO: 17, as shown in the table below:

| S protein RBD | |
|---|---|
| residue position | Amino acid residue |
| 3 | S |
| 6 | Q |
| 7 | E |
| 21 | D |
| 22 | K |
| 38 | T |
| 42 | D |
| 48 | T |
| 67 | S |
| 70 | I |
| 76 | S |
| 81 | T |
| 83 | L |
| 86 | C |
| 87 | S |
| 92 | V |
| 121 | A |
| 122 | K |
| 123 | Q |
| 125 | T |
| 126 | G |
| 128 | S |
| 134 | Y |
| 137 | S |
| 138 | H |
| 141 | T |
| 150 | L |
| 152 | S |
| 153 | D |
| 154 | E |
| 155 | C |
| 167 | F |
| 171 | R |
| 178 | T |
| 180 | S |
| 181 | T |
| 183 | D |
| 185 | N |
| 187 | N |
| 188 | V |
| 189 | P |
| 191 | E |
| 194 | A |
| 195 | T |
| 219 | Q |

5. An isolated polypeptide according to any preceding paragraph, which comprises amino acid residues at positions corresponding to the amino acid residue positions of SEQ ID NO: 17, as shown in the table below:

| S protein RBD | |
|---|---|
| residue position | Amino acid residue |
| 3 | S |
| 6 | Q |
| 7 | E |
| 21 | D |
| 22 | K |
| 38 | T |
| 42 | D |
| 48 | T |
| 67 | S |
| 70 | I |
| 76 | S |
| 81 | T |
| 83 | L |
| 86 | C |
| 87 | S |
| 92 | V |
| 121 | A |
| 122 | K |
| 123 | Q |
| 125 | T |
| 126 | G |
| 128 | S |
| 134 | Y |
| 137 | S |
| 138 | H |
| 141 | T |
| 150 | L |
| 152 | S |
| 153 | D |
| 154 | E |
| 155 | C |
| 167 | F |
| 171 | R |
| 178 | T |
| 180 | S |
| 181 | T |
| 183 | D |
| 185 | N |
| 187 | N |
| 188 | V |
| 189 | P |
| 191 | E |
| 194 | A |
| 195 | T |
| 219 | Q |

6. An isolated polypeptide according to paragraph 5, which comprises amino acid residues at positions corresponding to the amino acid residue positions of SEQ ID NO: 17, as shown in the table below:

| S protein RBD | |
|---|---|
| residue position | Amino acid residue |
| 3 | S |
| 6 | Q |
| 7 | E |
| 8 | V |
| 21 | D |
| 22 | K |
| 30 | P |
| 36 | E |
| 38 | T |
| 39 | K |
| 42 | D |
| 48 | T |
| 54 | T |
| 67 | S |
| 70 | I |
| 76 | S |
| 81 | T |
| 83 | L |
| 86 | C |
| 87 | S |
| 92 | V |
| 99 | V |
| 120 | T |
| 121 | A |
| 122 | K |
| 123 | Q |
| 125 | T |
| 126 | G |
| 127 | S |
| 128 | S |
| 134 | Y |
| 137 | S |
| 138 | H |
| 141 | T |
| 142 | K |
| 150 | L |
| 152 | S |
| 153 | D |
| 154 | E |
| 155 | C |
| 156 | S |
| 157 | P |
| 158 | D |
| 159 | G |
| 160 | K |
| 163 | T |
| 164 | P |
| 165 | P |
| 166 | A |
| 167 | F |
| 171 | R |
| 178 | T |
| 180 | S |
| 181 | T |
| 183 | D |
| 185 | N |
| 187 | N |
| 188 | V |
| 189 | P |
| 191 | E |
| 194 | A |
| 195 | T |
| 206 | N |
| 216 | L |
| 219 | Q |

7. An isolated polypeptide according to paragraph 5, which comprises amino acid residues at positions corresponding to the amino acid residue positions of SEQ ID NO:17, as shown in the table below:

| S protein RBD | Amino acid |
|---|---|
| residue position | residue |
| 3 | S |
| 5 | T |
| 6 | Q |
| 7 | E |
| 21 | D |
| 22 | K |
| 28 | R |
| 38 | T |
| 42 | D |
| 48 | T |
| 55 | S |
| 66 | P |
| 67 | S |
| 70 | I |
| 75 | T |
| 76 | S |
| 81 | T |
| 83 | L |
| 84 | I |
| 85 | R |
| 86 | C |
| 87 | S |
| 88 | E |
| 92 | V |
| 112 | T |
| 116 | I |
| 121 | A |
| 122 | K |
| 123 | Q |
| 125 | T |
| 126 | G |
| 128 | S |
| 134 | Y |
| 137 | S |
| 138 | H |
| 140 | K |
| 141 | T |
| 144 | K |
| 150 | L |
| 152 | S |
| 153 | D |
| 154 | E |
| 155 | C |
| 167 | F |
| 168 | N |
| 169 | G |
| 170 | V |
| 171 | R |
| 172 | G |
| 173 | F |
| 177 | F |
| 178 | T |
| 180 | S |
| 181 | T |
| 183 | D |
| 185 | N |
| 186 | P |
| 187 | N |
| 188 | V |
| 189 | P |
| 190 | V |
| 191 | E |
| 194 | A |
| 195 | T |
| 219 | Q |

8. A polypeptide according to any preceding paragraph, which comprises an amino acid sequence of SEQ ID NO:17.

9. An isolated polypeptide, which comprises a coronavirus S protein RBD domain with any, or all of the amino acid residues at positions corresponding to the amino acid residue positions as shown in the table below.

| S protein RBD | Amino acid |
|---|---|
| residue position | residue |
| 3 | S |
| 6 | Q |
| 7 | E |
| 21 | D |
| 22 | K |
| 38 | T |
| 42 | D |
| 48 | T |
| 67 | S |
| 70 | I |
| 76 | S |
| 81 | T |
| 83 | L |
| 86 | C |
| 87 | S |
| 92 | V |
| 121 | A |
| 122 | K |
| 123 | Q |
| 125 | T |
| 126 | G |
| 128 | S |
| 134 | Y |
| 137 | S |
| 138 | H |
| 141 | T |
| 150 | L |
| 152 | S |
| 153 | D |
| 154 | E |
| 155 | C |
| 167 | F |
| 171 | R |
| 178 | T |
| 180 | S |
| 181 | T |
| 183 | D |
| 185 | N |
| 187 | N |
| 188 | V |
| 189 | P |
| 191 | E |
| 194 | A |
| 195 | T |
| 219 | Q |

10. An isolated polypeptide according to paragraph 9, which comprises at least five, at least ten, at least fifteen, at least twenty, at least twenty five, at least thirty, at least thirty five, or at least forty of the amino acid residues at positions corresponding to the amino acid residue positions as shown in the table.

11. An isolated polypeptide, which comprises a coronavirus S protein RBD domain with any, or all of the amino acid residues at positions corresponding to the amino acid residue positions as shown in the table below:

| S protein RBD | Amino acid |
|---|---|
| residue position | residue |
| 3 | S |
| 6 | Q |
| 7 | E |
| 8 | V |
| 21 | D |
| 22 | K |
| 30 | P |
| 36 | E |
| 38 | T |
| 39 | K |
| 42 | D |
| 48 | T |
| 54 | T |
| 67 | S |
| 70 | I |
| 76 | S |
| 81 | T |
| 83 | L |
| 86 | C |
| 87 | S |
| 92 | V |
| 99 | V |
| 120 | T |
| 121 | A |
| 122 | K |
| 123 | Q |
| 125 | T |
| 126 | G |
| 127 | S |
| 128 | S |
| 134 | Y |
| 137 | S |
| 138 | H |
| 141 | T |
| 142 | K |
| 150 | L |
| 152 | S |
| 153 | D |
| 154 | E |
| 155 | C |
| 156 | S |
| 157 | P |
| 158 | D |
| 159 | G |
| 160 | K |
| 163 | T |
| 164 | P |
| 165 | P |
| 166 | A |
| 167 | F |
| 171 | R |
| 178 | T |
| 180 | S |
| 181 | T |
| 183 | D |
| 185 | N |
| 187 | N |
| 188 | V |
| 189 | P |
| 191 | E |
| 194 | A |
| 195 | T |
| 206 | N |
| 216 | L |
| 219 | Q |

12. An isolated polypeptide, which comprises a coronavirus S protein RBD domain with any, or all of the amino acid residues at positions corresponding to the amino acid residue positions as shown in the table below:

| S protein RBD | Amino acid |
|---|---|
| residue position | residue |
| 3 | S |
| 5 | T |
| 6 | Q |
| 7 | E |
| 21 | D |
| 22 | K |
| 28 | R |
| 38 | T |
| 42 | D |
| 48 | T |
| 55 | S |
| 66 | P |
| 67 | S |
| 70 | I |
| 75 | T |
| 76 | S |
| 81 | T |
| 83 | L |
| 84 | I |
| 85 | R |
| 86 | C |
| 87 | S |
| 88 | E |
| 92 | V |
| 112 | T |
| 116 | I |
| 121 | A |
| 122 | K |
| 123 | Q |
| 125 | T |
| 126 | G |
| 128 | S |
| 134 | Y |
| 137 | S |
| 138 | H |
| 140 | K |
| 141 | T |
| 144 | K |
| 150 | L |
| 152 | S |
| 153 | D |
| 154 | E |
| 155 | C |
| 167 | F |
| 168 | N |
| 169 | G |
| 170 | V |
| 171 | R |
| 172 | G |
| 173 | F |
| 177 | F |
| 178 | T |
| 180 | S |
| 181 | T |
| 183 | D |
| 185 | N |
| 186 | P |
| 187 | N |
| 188 | V |
| 189 | P |
| 190 | V |
| 191 | E |
| 194 | A |
| 195 | T |
| 219 | Q |

13. An isolated polypeptide, which comprises an amino acid sequence of SEQ ID NO: 27 (COV_S_T2_13), or an amino acid sequence which has at least 88%, 89%, 90%, 91%, 92%, 93%, 94%, 95%, 96%, 97%, 98%, or 99% amino acid identity over its entire length with the amino acid sequence of SEQ ID NO:27.

14. A polypeptide according to paragraph 13, which comprises at least one, or all of the amino acid residues at positions corresponding to the amino acid residue positions of SEQ ID NO: 11, as shown in the table below:

| SARS2 RBD | |
|---|---|
| residue position | |
| (CoV_T2_6; | |
| SEQ ID NO: 11) | Residue |
| 3 | A |
| 6 | K |
| 7 | E |
| 8 | V |
| 30 | P |
| 36 | E |
| 54 | T |
| 120 | T |
| 126 | T |
| 127 | T |
| 152 | S |
| 153 | D |
| 163 | S |
| 201 | N |
| 211 | L |
| 214 | D |

15. A polypeptide according to paragraph 13 or 14, which comprises at least one, or all of the amino acid residues at positions corresponding to the amino acid residue positions of SEQ ID NO: 11, as shown in the table below:

| SARS2 RBD | |
|---|---|
| residue position | |
| (CoV_T2_6; | |
| SEQ ID NO: 11) | Residue |
| 99 | V |
| 137 | S |
| 138 | L |
| 142 | K |
| 156 | S |
| 157 | P |
| 159 | G |
| 160 | K |
| 172 | Y |
| 175 | R |
| 180 | F |

16. An isolated polypeptide according to any of paragraphs 13 to 15, which comprises an amino acid sequence of SEQ ID NO: 27 (COV_S_T2_13).

17. An isolated polypeptide, which comprises an amino acid sequence of SEQ ID NO: 28 (COV_S_T2_14), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28.

18. A polypeptide according to paragraph 17, which comprises at least one, or all of the amino acid residues at positions corresponding to the amino acid residue positions of SEQ ID NO: 11, as shown in the table below:

| SARS2 RBD | |
|---|---|
| residue position | |
| (CoV_T2_6; | |
| SEQ ID NO: 11) | Residue |
| 3 | A |
| 6 | K |
| 7 | E |
| 8 | V |
| 30 | P |
| 36 | E |
| 54 | T |
| 120 | T |
| 126 | T |
| 127 | T |
| 152 | S |
| 153 | D |
| 163 | S |
| 201 | N |
| 211 | L |
| 214 | D |

19. A polypeptide according to paragraph 17 or 18, which comprises at least one, or all of the amino acid residues at positions corresponding to the amino acid residue positions of SEQ ID NO: 11, as shown in the table below:

| SARS2 RBD | |
|---|---|
| residue position | |
| (CoV_T2_6; | |
| SEQ ID NO: 11) | Residue |
| 99 | V |
| 137 | S |
| 138 | L |
| 142 | K |
| 156 | S |
| 157 | P |
| 159 | G |
| 160 | K |
| 172 | Y |
| 175 | R |
| 180 | F |

20. A polypeptide according to any of paragraphs 17 to 19, which comprises at least one, or all of the amino acid residues at positions corresponding to the amino acid residue positions of SEQ ID NO:11, as shown in the table below:

| SARS2 RBD | |
|---|---|
| (CoV_T2_6; | |
| SEQ ID NO: 11) | |
| residue position | Residue |
| 28 | K |
| 39 | K |
| 123 | I |

21. An polypeptide according to any of paragraphs 17 to 20, which comprises an amino acid sequence of SEQ ID NO: 28 (COV_S_T2_14).

22. An isolated polypeptide, which comprises an amino acid sequence of SEQ ID NO: 29 (COV_S_T2_15), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29.

23. A polypeptide according to paragraph 22, which comprises at least one, or all of the amino acid residues at positions corresponding to the amino acid residue positions of SEQ ID NO: 11, as shown in the table below:

| SARS2 RBD | |
|---|---|
| residue position | |
| (CoV_T2_6; | |
| SEQ ID NO: 11) | Residue |
| 3 | A |
| 6 | K |
| 7 | E |
| 8 | V |
| 30 | P |
| 36 | E |
| 54 | T |
| 120 | T |
| 126 | T |
| 127 | T |
| 152 | S |
| 153 | D |
| 163 | S |
| 201 | N |
| 211 | L |
| 214 | D |

24. A polypeptide according to paragraph 22 or 23, which comprises at least one, or all of the amino acid residues at positions corresponding to the amino acid residue positions of SEQ ID NO: 11, as shown in the table below:

| SARS2 RBD | |
|---|---|
| residue position | |
| (CoV_T2_6; | |
| SEQ ID NO: 11) | Residue |
| 99 | V |
| 137 | S |
| 138 | L |
| 142 | K |
| 156 | S |
| 157 | P |
| 159 | G |
| 160 | K |
| 172 | Y |
| 175 | R |
| 180 | F |

25. A polypeptide according to any of paragraphs 22 to 24, which comprises at least one, or all of the amino acid residues at positions corresponding to the amino acid residue positions of SEQ ID NO: 11, as shown in the table below:

| SARS2 RBD | |
|---|---|
| residue position | |
| (CoV_T2_6; | |
| SEQ ID NO: 11) | Residue |
| 55 | F |
| 75 | S |
| 112 | M |

26. An isolated polypeptide according to any of paragraphs 22 to 25, which comprises an amino acid sequence of SEQ ID NO: 29 (COV_S_T2_15).

27. An isolated polypeptide, which comprises an amino acid sequence of SEQ ID NO: 30 (COV_S_T2_16), or an amino acid sequence which has at least 93%, 94%, 95%, 96%, 97%, 98%, or 99% amino acid identity over its entire length with the amino acid sequence of SEQ ID NO: 30.

28. A polypeptide according to paragraph 27, which comprises at least one, or all of the amino acid residues at positions corresponding to the amino acid residue positions of SEQ ID NO: 11, as shown in the table below:

| SARS2 RBD | |
|---|---|
| residue position | |
| (CoV_T2_6; | |
| SEQ ID NO: 11) | Residue |
| 3 | A |
| 6 | K |
| 7 | E |
| 8 | V |
| 30 | P |
| 36 | E |
| 54 | T |
| 120 | T |
| 126 | T |
| 127 | T |
| 152 | S |
| 153 | D |
| 163 | S |
| 201 | N |
| 211 | L |
| 214 | D |

29. An isolated polypeptide according to paragraph 27 or 28, which comprises an amino acid sequence of SEQ ID NO: 30 (COV_S_T2_16).

30. An isolated polypeptide, which comprises an amino acid sequence of SEQ ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31.

31. A polypeptide according to paragraph 30, which comprises at least one, or all of the amino acid residues at positions corresponding to the amino acid residue positions of SEQ ID NO: 11, as shown in the table below:

| SARS2 RBD | |
|---|---|
| residue position | |
| (CoV_T2_6; | |
| SEQ ID NO: 11) | Residue |
| 3 | A |
| 6 | K |
| 7 | E |
| 8 | V |
| 30 | P |
| 36 | E |
| 54 | T |
| 120 | T |
| 126 | T |
| 127 | T |
| 152 | S |
| 153 | D |
| 163 | S |
| 201 | N |
| 211 | L |
| 214 | D |

32. A polypeptide according to paragraph 30 or 31, which comprises at least one, or all of the amino acid residues at positions corresponding to the amino acid residue positions of SEQ ID NO: 11, as shown in the table below:

| SARS2 RBD | |
|---|---|
| residue position | |
| (CoV_T2_6; | |
| SEQ ID NO: 11) | Residue |
| 99 | V |
| 137 | S |
| 138 | L |
| 142 | K |
| 156 | S |
| 157 | P |
| 159 | G |
| 160 | K |
| 172 | Y |
| 175 | R |
| 180 | F |

33. A polypeptide according to any of paragraphs 30 to 32, which comprises at least one, or all of the amino acid residues at positions corresponding to the amino acid residue positions of SEQ ID NO:11, as shown in the table below:

| SARS2 RBD | |
|---|---|
| residue position | |
| (CoV_T2_6; | |
| SEQ ID NO: 11) | Residue |
| 28 | K |
| 39 | K |
| 123 | I |

34. A polypeptide according to any of paragraphs 30 to 33, which comprises at least one, or all of the amino acid residues at positions corresponding to the amino acid residue positions of SEQ ID NO: 11, as shown in the table below:

| SARS2 RBD | |
|---|---|
| residue position | |
| (CoV_T2_6; | Design |
| SEQ ID NO: 11) | residue |
| 185 | T |

35. An isolated polypeptide according to any of paragraphs 30 to 34, which comprises an amino acid sequence of SEQ ID NO: 31 (COV_S_T2_17),

36. An isolated polypeptide, which comprises an amino acid sequence of SEQ ID NO: 32 (COV_S_T2_18), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32.

37. A polypeptide according to paragraph 36, which comprises at least one, or all of the amino acid residues at positions corresponding to the amino acid residue positions of SEQ ID NO: 11, as shown in the table below:

| SARS2 RBD | |
|---|---|
| residue position | |
| (CoV_T2_6; | |
| SEQ ID NO: 11) | Residue |
| 3 | A |
| 6 | K |
| 7 | E |
| 8 | V |
| 30 | P |
| 36 | E |
| 54 | T |
| 120 | T |
| 126 | T |
| 127 | T |
| 152 | S |
| 153 | D |
| 163 | S |
| 201 | N |
| 211 | L |
| 214 | D |

38. A polypeptide according to paragraph 36 or 37, which comprises at least one, or all of the amino acid residues at positions corresponding to the amino acid residue positions of SEQ ID NO: 11, as shown in the table below:

| SARS2 RBD | |
|---|---|
| residue position | |
| (CoV_T2_6; | |
| SEQ ID NO: 11) | Residue |
| 99 | V |
| 137 | S |
| 138 | L |
| 142 | K |
| 156 | S |
| 157 | P |
| 159 | G |
| 160 | K |
| 172 | Y |
| 175 | R |
| 180 | F |

39. A polypeptide according to any of paragraphs 36 to 38, which comprises at least one, or all of the amino acid residues at positions corresponding to the amino acid residue positions of SEQ ID NO:11, as shown in the table below:

| SARS2 RBD | |
|---|---|
| residue position | |
| (CoV_T2_6; | |
| SEQ ID NO: 11) | Residue |
| 55 | F |
| 75 | S |
| 112 | M |

40. A polypeptide according to any of paragraphs 36 to 39, which comprises at least one, or all of the amino acid residues at positions corresponding to the amino acid residue positions of SEQ ID NO:11, as shown in the table below:

| SARS2 RBD | |
|---|---|
| residue position | |
| (CoV_T2_6; | |
| SEQ ID NO: 11) | Residue |
| 185 | T |

41. An isolated polypeptide according to any of paragraphs 36 to 40, which comprises an amino acid sequence of SEQ ID NO: 32 (COV_S_T2_18).

42. An isolated polypeptide which comprises a coronavirus S protein RBD domain with at least one, at least five, at least ten, at least fifteen, or all of the amino acid residues at positions corresponding to the amino acid residue positions of SEQ ID NO:11, as shown in the table below:

| SARS2 RBD | |
|---|---|
| residue position | |
| (CoV_T2_6; | |
| SEQ ID NO: 11) | Residue |
| 3 | A |
| 6 | K |
| 7 | E |
| 8 | V |
| 30 | P |
| 36 | E |
| 54 | T |
| 120 | T |
| 126 | T |
| 127 | T |
| 152 | S |
| 153 | D |
| 163 | S |
| 201 | N |
| 211 | L |
| 214 | D |

43. An isolated polypeptide according to paragraph 42, which further comprises at least one, or all of the amino acid residues at positions corresponding to the amino acid residue positions of SEQ ID NO:11, as shown in the table below:

| SARS2 RBD | |
|---|---|
| residue position | |
| (CoV_T2_6; | |
| SEQ ID NO: 11) | Residue |
| 99 | V |
| 137 | S |
| 138 | L |
| 142 | K |
| 156 | S |
| 157 | P |
| 159 | G |
| 160 | K |
| 172 | Y |
| 175 | R |
| 180 | F |

44. An isolated polypeptide according to paragraph 42 or 43, which further comprises at least one, or all of the amino acid residues at positions corresponding to the amino acid residue positions of SEQ ID NO:11, as shown in the table below:

| SARS2 RBD | |
|---|---|
| (CoV_T2_6; | |
| SEQ ID NO: 11) | |
| residue position | Residue |
| 28 | K |
| 39 | K |
| 123 | I |

45. An isolated polypeptide according to any of paragraphs 42 to 44, which further comprises at least one, or all of the amino acid residues at positions corresponding to the amino acid residue positions of SEQ ID NO: 11, as shown in the table below:

| SARS2 RBD | |
|---|---|
| residue position | |
| (CoV_T2_6; | |
| SEQ ID NO: 11) | Residue |
| 55 | F |
| 75 | S |
| 112 | M |

46. An isolated polypeptide according to any of paragraphs 42 to 45, which further comprises at least one, or all of the amino acid residues at positions corresponding to the amino acid residue positions of SEQ ID NO: 11, as shown in the table below:

| SARS2 RBD | |
|---|---|
| residue position | |
| (CoV_T2_6; | |
| SEQ ID NO: 11) | Residue |
| 185 | T |

47. An isolated polypeptide, which comprises an amino acid sequence of SEQ ID NO:33.

48. An isolated polypeptide, which comprises an amino acid sequence of a SARS2 RBD with a glycosylation site located within the last 10 amino acids of the SARS2 RBD sequence, preferably at residue position 203.

49. An isolated polypeptide, which comprises an amino acid sequence of SEQ ID NO:34, or an amino acid sequence which has at least 99% amino acid identity over its entire length with the amino acid sequence of SEQ ID NO:34.

50. A polypeptide according to paragraph 49, which comprises at least one, or all of the following amino acid residues at positions corresponding to the amino acid residue positions of SEQ ID NO:11: 13Q, 25Q, 54T.

51. An isolated polypeptide which comprises a coronavirus S protein RBD domain with at least one of the following amino acid residues at positions corresponding to the amino acid residue positions of SEQ ID NO: 11: 13Q, 25Q, 54T, 203N.

52. An isolated polypeptide comprising an amino acid sequence of SEQ ID NO:35 (M9), or an amino acid sequence which has at least 70% amino acid identity over its entire length with the amino acid sequence of SEQ ID NO:35.

53. A polypeptide according to paragraph 52, which comprises at least one, or all of the amino acid residues at positions corresponding to the amino acid residue positions as shown in the table below:

| SARS2 RBD | |
|---|---|
| (SEQ ID | |
| NO: 11) | M9 residue |
| residue | (SEQ ID |
| position | NO: 35) |
| 3 | S |
| 6 | Q |
| 7 | E |
| 8 | V |
| 21 | D |
| 22 | D |
| 30 | P |
| 36 | E |
| 38 | T |
| 39 | T |
| 42 | D |
| 48 | T |
| 54 | T |
| 67 | S |
| 70 | I |
| 76 | S |
| 81 | T |
| 83 | L |
| 86 | C |
| 87 | S |
| 92 | V |
| 99 | V |
| 120 | T |
| 121 | A |
| 122 | K |
| 123 | Q |
| 125 | T |
| 126 | G |
| 127 | S |
| 128 | S |
| 134 | Y |
| 137 | S |
| 138 | H |
| 141 | T |
| 142 | K |
| 150 | L |
| 152 | S |
| 153 | D |
| 154 | E |
| 155 | C |
| 156 | S |
| 157 | P |
| 158 | D |
| 159 | G |
| 160 | K |
| * | T |
| * | P |
| * | P |
| * | A |
| * | F |
| 166 | R |
| 173 | T |
| 175 | S |
| 176 | T |
| 178 | D |
| 180 | N |
| 182 | N |
| 183 | V |
| 184 | P |
| 186 | E |
| 189 | A |
| 190 | T |
| 201 | N |
| 203 | N |
| 211 | L |
| 214 | Q |

* Residues for insertion between amino acid residue positions 162 and 163 of SEQ ID NO: 11.

54. A polypeptide according to paragraph 52 or 53 54, which comprises at least one, or both of the following amino acid residues at positions corresponding to the amino acid residue positions of SEQ ID NO: 11: 54T, 203N.

55. A polypeptide according to any of paragraphs 52 to 54, which comprises an amino acid sequence of SEQ ID NO:35 (M9).

56. An isolated polypeptide comprising an amino acid sequence of SEQ ID NO:36 (M10), or an amino acid sequence which has at least 69% amino acid identity over its entire length with the amino acid sequence of SEQ ID NO:36.

57. A polypeptide according to paragraph 56, which comprises at least one, or all of the amino acid residues at positions corresponding to the amino acid residue positions as shown in the table below:

| SARS2 RBD | |
|---|---|
| (SEQ ID | |
| NO: 11) | M10 residue |
| residue | (SEQ ID |
| position | NO: 36) |
| 3 | S |
| 6 | Q |
| 7 | E |
| 8 | V |
| 13 | Q |
| 21 | K |
| 22 | K |
| 25 | Q |
| 30 | P |
| 36 | E |
| 38 | K |
| 39 | K |
| 42 | D |
| 48 | T |
| 54 | T |
| 67 | S |
| 70 | I |
| 76 | S |
| 81 | T |
| 83 | L |
| 86 | C |
| 87 | S |
| 92 | V |
| 99 | V |
| 120 | T |
| 121 | A |
| 122 | K |
| 123 | Q |
| 125 | T |
| 126 | G |
| 127 | S |
| 128 | S |
| 134 | Y |
| 137 | S |
| 138 | H |
| 141 | T |
| 142 | K |
| 150 | L |
| 152 | S |
| 153 | D |
| 154 | E |
| 155 | C |
| 156 | S |
| 157 | P |
| 158 | D |
| 159 | G |
| 160 | K |
| * | T |
| * | P |
| * | P |
| * | A |
| * | F |
| 166 | R |
| 173 | T |
| 175 | S |
| 176 | T |
| 178 | D |
| 180 | N |
| 182 | N |
| 183 | V |
| 184 | P |
| 186 | E |
| 189 | A |
| 190 | T |
| 201 | N |
| 211 | L |
| 214 | Q |

* Residues for insertion between amino acid residue positions 162 and 163 of SEQ ID NO: 11.

58. A polypeptide according to paragraph 56 or 578, which comprises at least one, or all of the following amino acid residues at positions corresponding to the amino acid residue positions of SEQ ID NO: 11: 13Q, 25Q, 54T.

59. An polypeptide according to any of paragraphs 56 to 58, which comprises an amino acid sequence of SEQ ID NO:36 (M10).

60. A polypeptide according to any preceding paragraph, which comprises at least one glycosylation site within amino acid sequence of the receptor binding domain (RBD).

61. A polypeptide according to any preceding paragraph, which comprises a glycosylation site located within the last 10 amino acids of amino acid sequence of the RBD, preferably at a residue position corresponding to residue position 203 of the RBD sequence.

62. An isolated polypeptide which comprises an amino acid sequence of SEQ ID NO:22, or an amino acid sequence which has at least 95%, 96%, 97%, 98%, or 99% amino acid identity over its entire length with the amino acid sequence of SEQ ID NO:22.

63. An isolated polypeptide according to paragraph 62, which comprises amino acid residues at positions corresponding to the amino acid residue positions of SEQ ID NO:22, as shown in the table below:

| SARS2 E protein | COV_E_T2_1 Amino |
|---|---|
| residue position | acid residue |
| 36 | A |
| 55 | T |

64. An isolated polypeptide according to paragraph 63, which comprises amino acid residues at positions corresponding to the amino acid residue positions of SEQ ID NO:22, as shown in the table below:

| SARS2 E protein | COV_E_T2_1 Amino |
|---|---|
| residue position | acid residue |
| 36 | A |
| 55 | T |
| 69 | Q |
| 70 | G |

65. An isolated polypeptide, which comprises an amino acid sequence of SEQ ID NO:23, or an amino acid sequence which has at least 98%, or 99% amino acid identity over its entire length with the amino acid sequence of SEQ ID NO:23.

66. An isolated polypeptide according to paragraph 65, which comprises amino acid residues at positions corresponding to the amino acid residue positions of SEQ ID NO:23, as shown in the table below:

| SARS2 E protein | COV_E_T2_2 Amino |
|---|---|
| residue position | acid residue |
| 36 | A |
| 55 | T |

67. An isolated polypeptide, which comprises an amino acid sequence of SEQ ID NO:42 (COV_E_T2_3), or an amino acid sequence which has at least 99% amino acid identity over its entire length with the amino acid sequence of SEQ ID NO:42.

68. A polypeptide according to paragraph 67, which comprises amino acid residue A at a position corresponding to amino acid residue position 15 of SEQ ID NO:41.

69. An isolated polypeptide comprising an amino acid sequence of SEQ ID NO:43 (COV_E_T2_4), or an amino acid sequence which has at least 95%, 96%, 97%, 98%, or 99% amino acid identity over its entire length with the amino acid sequence of SEQ ID NO:43.

70. A polypeptide according to paragraph 69, which comprises at least one, or all of the following amino acid residues at positions corresponding to the amino acid residue positions of SEQ ID NO:41: 15A, 55T, 69Q, 70G.

71 An isolated polypeptide, which comprises an amino acid sequence of SEQ ID NO:44 (COV_E_T2_5), or an amino acid sequence which has at least 98%, or 99% amino acid identity over its entire length with the amino acid sequence of SEQ ID NO:44.

72. A polypeptide according to paragraph 71, which comprises at least one, or all of the following amino acid residues at positions corresponding to the amino acid residue positions of SEQ ID NO:41: 15A, 55T.

73. An isolated polypeptide which comprises a coronavirus E protein with at least one of the following amino acid residues at positions corresponding to the amino acid residue positions of SEQ ID NO:41: 15A, 55T, 69Q, 70G.

74. An isolated polypeptide according to paragraph 73, which comprises at least one, or all of the following amino acid residues at positions corresponding to the amino acid residue positions of SEQ ID NO:41: 15A, 55T.

75. An isolated polypeptide which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO: 24.

76. An isolated polypeptide according to paragraph 75, which comprises amino acid residues at positions corresponding to the amino acid residue positions of SEQ ID NO:26, as shown in the table below:

| SARS2 M protein | COV_M_T2 1 Amino |
|---|---|
| residue position | acid residue |
| 40 | S |
| 76 | V |
| 87 | I |
| 97 | V |
| 125 | R |
| 134 | M |
| 151 | M |
| 155 | S |
| 197 | N |

77. An isolated polypeptide according to paragraph 75, which comprises amino acid residues at positions corresponding to the amino acid residue positions of SEQ ID NO:26, as shown in the table below:

| SARS2 M protein | COV_M_T2_1 Amino |
|---|---|
| residue position | acid residue |
| 4 | — (deletion) |
| 15 | Q |
| 30 | A |
| 33 | M |
| 40 | S |
| 52 | V |
| 76 | V |
| 87 | I |
| 97 | V |
| 125 | R |
| 134 | M |
| 145 | I |
| 151 | M |
| 155 | S |
| 188 | G |
| 189 | T |
| 197 | N |
| 211 | A |
| 212 | G |
| 214 | N |

78. An isolated polypeptide which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

79. An isolated polypeptide according to paragraph 78, which comprises amino acid residues at positions corresponding to the amino acid residue positions of SEQ ID NO:25, as shown in the table below:

| SARS2 M protein | COV_M_T2_2 Amino |
|---|---|
| residue position | acid residue |
| 40 | S |
| 76 | V |
| 87 | I |
| 97 | V |
| 125 | R |
| 134 | M |
| 151 | M |
| 155 | S |
| 197 | N |

80. An isolated polypeptide according to paragraph 78, which comprises amino acid residues at positions corresponding to the amino acid residue positions of SEQ ID NO:25, as shown in the table below:

| SARS2 M protein | COV_M_T2_2 Amino |
|---|---|
| residue position | acid residue |
| 40 | S |
| 76 | V |
| 87 | I |
| 97 | V |
| 125 | R |
| 127 | S |
| 134 | M |
| 151 | M |
| 155 | S |
| 189 | S |
| 195 | V |
| 197 | N |

81. An isolated polypeptide which comprises an amino acid sequence of SEQ ID NO:48, or an amino acid sequence which has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO: 48.

82. A polypeptide according to paragraph 81, which comprises a deletion of amino acid residues at positions corresponding to positions 20-75 of SEQ ID NO:26.

83. A polypeptide according to paragraph 81 or 82, which comprises amino acid residue G at a position corresponding to amino acid residue position 204 of SEQ ID NO:26.

84. An isolated polypeptide which comprises an amino acid sequence of SEQ ID NO:49, or an amino acid sequence which has at least 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:49.

85. A polypeptide according to paragraph 84, which comprises a deletion of amino acid residues at positions corresponding to positions 20-75 of SEQ ID NO:26.

86. A polypeptide according to paragraph 84 or 85, which comprises at least one, or all, of the amino acid residues, at a position corresponding to the amino acid residue position of SEQ ID NO: 26, as shown in the table below:

| SARS2 M protein | |
|---|---|
| residue position | Amino acid |
| (SEQ ID NO: 26) | residue |
| 20-75 | Deleted |
| 76 | V |
| 87 | I |
| 97 | V |
| 125 | R |
| 134 | M |
| 151 | M |
| 155 | S |
| 189 | T |
| 197 | N |
| 204 | G |

87. A polypeptide according to paragraph 86, which comprises at least five of the amino acid residues, at positions corresponding to the amino acid residue positions of SEQ ID NO: 26, as shown in the table.

88. A polypeptide according to paragraph 84 or 85, which comprises at least one, or all, of the amino acid residues, at a position corresponding to the amino acid residue position of SEQ ID NO: 26, as shown in the table below:

| | COV_M_T2_4 |
|---|---|
| SARS2 M protein | Amino acid |
| residue position | residue (SEQ |
| (SEQ ID NO: 26) | ID NO: 49) |
| 4 | Deleted |
| 15 | Q |
| 20-75 | Deleted |
| 76 | V |
| 87 | I |
| 97 | V |
| 125 | R |
| 134 | M |
| 145 | I |
| 151 | M |
| 155 | S |
| 188 | G |
| 189 | T |
| 197 | N |
| 204 | G |
| 211 | A |
| 212 | G |
| 214 | N |

89. A polypeptide according to paragraph 88, which comprises at least five, at least ten, or at least fifteen of the amino acid residues, at positions corresponding to the amino acid residue positions of SEQ ID NO:26, as shown in the table.

90. An isolated polypeptide which comprises an amino acid sequence of SEQ ID NO:50, or an amino acid sequence which has at least 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:50.

91. A polypeptide according to paragraph 90, which comprises a deletion of amino acid residues at positions corresponding to positions 20-75 of SEQ ID NO:26.

92. A polypeptide according to paragraph 90 or 91, which comprises at least one, or all, of the amino acid residues, at a position corresponding to the amino acid residue position of SEQ ID NO: 26, as shown in the table below:

| SARS2 M protein | |
|---|---|
| residue position | Amino acid |
| (SEQ ID NO: 26) | residue |
| 20-75 | Deleted |
| 76 | V |
| 87 | I |
| 97 | V |
| 125 | R |
| 134 | M |
| 151 | M |
| 155 | S |
| 189 | T |
| 197 | N |
| 204 | G |

93. A polypeptide according to paragraph 92, which comprises at least five of the amino acid residues, at positions corresponding to the amino acid residue positions of SEQ ID NO: 26, as shown in the table.

94. A polypeptide according to paragraph 90 or 91, which comprises at least one, or all, of the amino acid residues, at a position corresponding to the amino acid residue position of SEQ ID NO: 26, as shown in the table below:

| | COV_M_T2_5 |
|---|---|
| SARS2 M protein | Amino acid |
| residue position | residue (SEQ |
| (SEQ ID NO: 26) | ID NO: 50) |
| 20-75 | Deleted |
| 76 | V |
| 87 | I |
| 97 | V |
| 125 | R |
| 127 | S |
| 129 | I |
| 134 | M |
| 151 | M |
| 155 | S |
| 189 | S |
| 195 | V |
| 197 | N |
| 204 | G |

95. A polypeptide according to paragraph 94, which comprises at least five or at least ten of the amino acid residues, at positions corresponding to the amino acid residue positions of SEQ ID NO:26, as shown in the table.

96. An isolated polypeptide, which comprises a coronavirus M protein with any, or all of the amino acid residues at positions corresponding to the amino acid residue positions as shown in the table below:

| SARS2 M protein | |
|---|---|
| residue position | Amino acid |
| (SEQ ID NO: 26) | residue |
| 20-75 | Deleted |
| 76 | V |
| 87 | I |
| 97 | V |
| 125 | R |
| 134 | M |
| 151 | M |
| 155 | S |
| 189 | T |
| 197 | N |
| 204 | G |

97. A polypeptide according to paragraph 96, which comprises at least five of the amino acid residues, at positions corresponding to the amino acid residue positions of SEQ ID NO: 26, as shown in the table.

98. An isolated polypeptide, which comprises a coronavirus M protein with any, or all of the amino acid residues at positions corresponding to the amino acid residue positions as shown in the table below:

| | COV_M_T2_4 |
|---|---|
| SARS2 M protein | Amino acid |
| residue position | residue (SEQ |
| (SEQ ID NO: 26) | ID NO: 49) |
| 4 | Deleted |
| 15 | Q |
| 20-75 | Deleted |
| 76 | V |
| 87 | I |
| 97 | V |
| 125 | R |
| 134 | M |
| 145 | I |
| 151 | M |
| 155 | S |
| 188 | G |
| 189 | T |
| 197 | N |
| 204 | G |
| 211 | A |
| 212 | G |
| 214 | N |

99. A polypeptide according to paragraph 98, which comprises at least five, at least ten, or at least fifteen of the amino acid residues, at positions corresponding to the amino acid residue positions of SEQ ID NO:26, as shown in the table.

100. An isolated polypeptide, which comprises a coronavirus M protein with any, or all of the amino acid residues at positions corresponding to the amino acid residue positions as shown in the table below:

| | COV_M_T2_5 |
|---|---|
| SARS2 M protein | Amino acid |
| residue position | residue (SEQ |
| (SEQ ID NO: 26) | ID NO: 50) |
| 20-75 | Deleted |
| 76 | V |
| 87 | I |
| 97 | V |
| 125 | R |
| 127 | S |
| 129 | I |
| 134 | M |
| 151 | M |
| 155 | S |
| 189 | S |
| 195 | V |
| 197 | N |
| 204 | G |

101. A polypeptide according to paragraph 100, which comprises at least five or at least ten of the amino acid residues, at positions corresponding to the amino acid residue positions of SEQ ID NO:26, as shown in the table.

102. An isolated polypeptide which comprises an amino acid sequence of SEQ ID NO:46 (COV_N_T2_1), or an amino acid sequence which has at least 93%, 94%, 95%, 96%, 97%, 98%, or 99% amino acid identity over its entire length with the amino acid sequence of SEQ ID NO: 46.

103. A polypeptide according to paragraph 102, which further comprises at least one, or all of the amino acid residues at positions corresponding to the amino acid residue positions as shown in Table 12.2 above.

104. A polypeptide according to paragraph 103, which comprises at least five, at least ten, or at least fifteen of the amino acid residues at positions corresponding to the amino acid residue positions as shown in the table.

105. A polypeptide according to any of paragraphs 102 to 104, which further comprises at least one, or all of the amino acid residues at positions corresponding to the amino acid residue positions as shown in Table 12.3 above.

106. A polypeptide according to paragraph 105, which comprises at least five or at least ten of the amino acid residues at positions corresponding to the amino acid residue positions as shown in the table.

107. An isolated polypeptide which comprises an amino acid sequence of SEQ ID NO:47 (COV_N_T2_2), or an amino acid sequence which has at least 92%, 93%, 94%, 95%, 96%, 97%, 98%, or 99% amino acid identity over its entire length with the amino acid sequence of SEQ ID NO:47.

108. A polypeptide according to paragraph 107, which further comprises at least one, or all of the amino acid residues at positions corresponding to the amino acid residue positions as shown in Table 12.2 above.

109. A polypeptide according to paragraph 108, which comprises at least five, at least ten, or at least fifteen of the amino acid residues at positions corresponding to the amino acid residue positions as shown in the table.

110. A polypeptide according to any of paragraphs 107 to 109, which further comprises at least one, or all of the amino acid residues at positions corresponding to the amino acid residue positions as shown in Table 12.4 above.

111. A polypeptide according to paragraph 110, which comprises at least five, at least ten, or at least fifteen of the amino acid residues at positions corresponding to the amino acid residue positions as shown in the table.

112. An isolated polypeptide, which comprises a coronavirus N protein with at least one, or all of the amino acid residues at positions corresponding to the amino acid residue positions of SEQ ID NO:45 as shown in Table 12.2 above.

113. An isolated polypeptide according to paragraph 112, which comprises at least five, at least ten, or at least fifteen amino acid residues at positions corresponding to the amino acid residue positions of SEQ ID NO:45, as shown in Table 12.2 above.

114. An isolated polypeptide according to paragraph 112 or 113, which comprises at least one, or all of the amino acid residues at positions corresponding to the amino acid residue positions of SEQ ID NO:45, as shown in Table 12.3 above.

115. An isolated polypeptide according to paragraph 114, which comprises at least five, or at least ten, of the amino acid residues at positions corresponding to the amino acid residue positions of SEQ ID NO:45, as shown in Table 12.3 above.

116. An isolated polypeptide according to paragraph 114 or 115, which comprises at least one, or all of the amino acid residues at positions corresponding to the amino acid residue positions of SEQ ID NO:45, as shown in Table 12.4 above.

117. An isolated polypeptide according to paragraph 116, which comprises at least five, at least ten, or at least fifteen, of the amino acid residues at positions corresponding to the amino acid residue positions of SEQ ID NO:45, as shown in Table 12.4 above.

118. An isolated polypeptide comprising an amino acid sequence of SEQ ID NO:5.

119. An isolated polypeptide comprising an amino acid sequence of SEQ ID NO: 11.

120. An isolated polypeptide, which comprises an amino acid sequence of SEQ ID NO:53, or an amino acid sequence which has at least 99% amino acid identity over its entire length with the amino acid sequence of SEQ ID NO:53.

121. An isolated polypeptide according to paragraph 120, which comprises at least one, or all of the amino acid residues or deletions, at positions corresponding to the amino acid residue positions of SEQ ID NO:52, as shown in the Table below:

| SARS2 S | |
|---|---|
| protein | |
| residue | |
| position | COV_S_T2_29 |
| (SEQ ID | amino acid residue |
| NO: 52) | (SEQ ID NO: 53) |
| 18 | F |
| 20 | N |
| 26 | S |
| 69 | — (deletion) |
| 70 | — (deletion) |
| 144 | — (deletion) |
| 417 | N |
| 484 | K |
| 501 | Y |
| 614 | G |
| 681 | H |
| 986 | P |
| 987 | P |

122. A polypeptide according to paragraph 121, which comprises at least five or at least ten of the amino acid residues or deletions, at positions corresponding to the amino acid residue positions of SEQ ID NO:52, as shown in the table.

123. An isolated polypeptide according to any of paragraphs 120 to 122, which comprises amino acid residue P at position 986, and amino acid residue P at position 987, corresponding to the amino acid residue positions of SEQ ID NO:52, and at least one, or all of the amino acid residues or deletions, at positions corresponding to the amino acid residue positions of SEQ ID NO: 52, as shown in the Table below:

| SARS2 S | |
|---|---|
| protein | |
| residue | |
| position | COV_S_T2_29 |
| (SEQ ID | amino acid residue |
| NO: 52) | (SEQ ID NO: 53) |
| 18 | F |
| 20 | N |
| 26 | S |
| 69 | — (deletion) |
| 70 | — (deletion) |
| 144 | — (deletion) |
| 417 | N |
| 484 | K |
| 501 | Y |
| 614 | G |
| 681 | H |

124. A polypeptide according to paragraph 123, which comprises at least five or at least ten of the amino acid residues or deletions, at positions corresponding to the amino acid residue positions of SEQ ID NO:52, as shown in the table.

125. An isolated polypeptide, which comprises a coronavirus S protein with at least one, or all of the amino acid residues or deletions, at positions corresponding to the amino acid residue positions of SEQ ID NO:52, as shown in the Table below:

| SARS2 S | |
|---|---|
| protein | |
| residue | |
| position | COV_S_T2_29 |
| (SEQ ID | amino acid residue |
| NO: 52) | (SEQ ID NO: 53) |
| 18 | F |
| 20 | N |
| 26 | S |
| 69 | — (deletion) |
| 70 | — (deletion) |
| 144 | — (deletion) |
| 417 | N |
| 484 | K |
| 501 | Y |
| 614 | G |
| 681 | H |
| 986 | P |
| 987 | P |

126. A polypeptide according to paragraph 125, which comprises at least five or at least ten of the amino acid residues or deletions, at positions corresponding to the amino acid residue positions of SEQ ID NO:52, as shown in the table.

127. An isolated polypeptide according to paragraph 125 or 126, which comprises amino acid residue P at position 986, and amino acid residue P at position 987, corresponding to the amino acid residue positions of SEQ ID NO:52, and at least one, or all of the amino acid residues or deletions, at positions corresponding to the amino acid residue positions of SEQ ID NO: 52, as shown in the Table below:

| SARS2 S protein | COV_S_T2_29 |
|---|---|
| residue position | amino acid residue |
| (SEQ ID NO: 52) | (SEQ ID NO: 53) |
| 18 | F |
| 20 | N |
| 26 | S |
| 69 | — (deletion) |
| 70 | — (deletion) |
| 144 | — (deletion) |
| 417 | N |
| 484 | K |
| 501 | Y |
| 614 | G |
| 681 | H |

128. A polypeptide according to paragraph 127, which comprises at least five or at least ten of the amino acid residues or deletions, at positions corresponding to the amino acid residue positions of SEQ ID NO:52, as shown in the table.

129. An isolated polypeptide according to any of paragraphs 125 to 128, wherein the coronavirus S protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:52.

130. An isolated polypeptide, which comprises an amino acid sequence of SEQ ID NO:54, or an amino acid sequence which has at least 99% amino acid identity over its entire length with the amino acid sequence of SEQ ID NO:54.

131. An isolated polypeptide according to paragraph 130, which comprises cysteine amino acid residues at positions corresponding to positions 413 and 987 of SEQ ID NO:52, and at least one, or all of the amino acid residues or deletions, at positions corresponding to the amino acid residue positions of SEQ ID NO:52, as shown in the Table below:

| SARS2 S protein | |
|---|---|
| residue position | |
| (SEQ ID NO: 52) | SEQ ID NO: 54 |
| 18 | F |
| 20 | N |
| 26 | S |
| 69 | — (deletion) |
| 70 | — (deletion) |
| 144 | — (deletion) |
| 417 | N |
| 484 | K |
| 501 | Y |
| 614 | G |
| 681 | H |

132. A polypeptide according to paragraph 131, which comprises at least five or at least ten of the amino acid residues or deletions, at positions corresponding to the amino acid residue positions of SEQ ID NO:52, as shown in the table.

133. An isolated polypeptide according to any of paragraphs 130 to 132, which comprises amino acid residue P at a position corresponding to position 986 of SEQ ID NO:52.

134. An isolated polypeptide, which comprises a coronavirus S protein comprising cysteine amino acid residues at positions corresponding to positions 413 and 987 of SEQ ID NO:52, and at least one or all of the amino acid residues or deletions at positions corresponding to the amino acid residue positions of SEQ ID NO:52, as shown in the Table below:

| SARS2 S protein | |
|---|---|
| residue position | |
| (SEQ ID NO: 52) | SEQ ID NO: 54 |
| 18 | F |
| 20 | N |
| 26 | S |
| 69 | — (deletion) |
| 70 | — (deletion) |
| 144 | — (deletion) |
| 417 | N |
| 484 | K |
| 501 | Y |
| 614 | G |
| 681 | H |

135. A polypeptide according to paragraph 134, which comprises at least five or at least ten of the amino acid residues or deletions, at positions corresponding to the amino acid residue positions of SEQ ID NO:52, as shown in the table.

136. An isolated polypeptide according to paragraph 134 or 135, which comprises amino acid residue residue P at a position corresponding to position 986 of SEQ ID NO:52.

137. An isolated polypeptide according to any of paragraphs 134 to 136, wherein the coronavirus S protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:52.

138. An isolated polypeptide according to any of paragraphs 1-61, or 118-137, which comprises an amino acid change at one or more (or all) positions corresponding to the following amino acid residue positions of SEQ ID NO:52: G446, L452, S477, and Q498.

139. An isolated polypeptide according to paragraph 138, which comprises one or more (or all) of the following amino acid residues at positions corresponding to the amino acid residue positions of SEQ ID NO:52: 446R, 477N, and 498R.

140. An isolated polypeptide according to paragraph 138 or 139, which comprises the following amino acid residues at positions corresponding to the amino acid residue positions of SEQ ID NO:52: 498R and 501Y.

141. A polypeptide according to any of paragraphs 17-21, which comprises the following discontinuous amino acid sequences:

(i)
(SEQ ID NO: 57)
NITNLCPFGEVFNATK;
(ii)
(SEQ ID NO: 58)
KKISN;
(iii)
(SEQ ID NO: 59)
NI

142. A polypeptide according to paragraph 141, wherein the discontinuous amino acid sequences (i), (ii), and (iii) are at amino acid residue positions corresponding to (i) residues 13-28; (ii) residues 38-42; and (iii) residues 122-123 of SEQ ID NO:28, respectively.

143. A polypeptide according to any of paragraphs 22-26, which comprises the following discontinuous amino acid sequences:

(i)
(SEQ ID NO: 60)
YNSTFFSTFKCYGVSPTKLNDLCFS;
(ii)
(SEQ ID NO: 61)
DDFM;
(iii)
(SEQ ID NO: 62)
FELLN.

144. A polypeptide according to paragraph 143, wherein the discontinuous amino acid sequences (i), (ii), and (iii) are at amino acid residue positions corresponding to (i) residues 51-75; (ii) residues 109-112; and (iii) residues 197-201 of SEQ ID NO:29, respectively.

145. A polypeptide according to any of paragraphs 27-29, which comprises the following discontinuous amino acid sequences:

(i)
(SEQ ID NO: 63)
RGDEVRQ;
(ii)
(SEQ ID NO: 64)
TGKIADY;
(iii)
(SEQ ID NO: 65)
YRLFRKSN;
(iv)
(SEQ ID NO: 66)
YQAGST;
(v)
(SEQ ID NO: 67)
FNCYFPLQSYGFQPTNGVGY.

146. A polypeptide according to paragraph 145, wherein the discontinuous amino acid sequences (i), (ii), (iii), (iv), and (v) are at amino acid residue positions corresponding to (i) residues 85-91, (ii) residues 97-103, (iii) residues 135-142, (iv) residues 155-160, and (v) residues 168-187 of SEQ ID NO:30, respectively.

147. A polypeptide according to any of paragraphs 30-35, which comprises the following discontinuous amino acid sequences:

(i)
(SEQ ID NO: 57)
NITNLCPFGEVFNATK;
(ii)
(SEQ ID NO: 58)
KKISN;
(iii)
(SEQ ID NO: 59)
NI.

148. A polypeptide according to paragraph 147, wherein the discontinuous amino acid sequences (i), (ii), and (iii) are at amino acid residue positions corresponding to (i) residues 13-28; (ii) residues 38-42; and (iii) residues 122-123 of SEQ ID NO:31, respectively.

149. A polypeptide according to any of paragraphs 36-41, which comprises the following discontinuous amino acid sequences:

(i)
(SEQ ID NO: 60)
YNSTFFSTFKCYGVSPTKLNDLCFS;
(ii)
(SEQ ID NO: 61)
DDFM;
(iii)
(SEQ ID NO: 62)
FELLN.

150. A polypeptide according to paragraph 149, wherein the discontinuous amino acid sequences (i), (ii), and (iii) are at amino acid residue positions corresponding to (i) residues 51-75; (ii) residues 109-112; and (iii) residues 197-201 of SEQ ID NO:32, respectively.

151. A polypeptide according to any of paragraphs 22-26, which comprises the following discontinuous amino acid sequences:

(i)
(SEQ ID NO: 68)
NITNLCPFGEVFNATR;
(ii)
(SEQ ID NO: 69)
KRISN;
(iii)
(SEQ ID NO: 70)
NL

152. A polypeptide according to paragraph 151, wherein the discontinuous amino acid sequences (i), (ii), and (iii) are at amino acid residue positions corresponding to (i) residues 13-28; (ii) residues 38-42; and (iii) residues 122-123 of SEQ ID NO:29, respectively.

153. A polypeptide according to any of paragraphs 27-29, which comprises the following discontinuous amino acid sequences:

(i)
(SEQ ID NO: 68)
NITNLCPFGEVFNATR;
(ii)
(SEQ ID NO: 69)
KRISN;
(iii)
(SEQ ID NO: 70)
NL

154. A polypeptide according to paragraph 153, wherein the discontinuous amino acid sequences (i), (ii), and (iii) are at amino acid residue positions corresponding to (i) residues 13-28; (ii) residues 38-42; and (iii) residues 122-123 of SEQ ID NO:30, respectively.

155. An isolated according to any of paragraphs 36-41, which comprises the following discontinuous amino acid sequences:

(i)
(SEQ ID NO: 68)
NITNLCPFGEVENATR;
(ii)
(SEQ ID NO: 69)
KRISN;
(iii)
(SEQ ID NO: 70)
NL

156. A polypeptide according to paragraph 155, wherein the discontinuous amino acid sequences (i), (ii), and (iii) are at amino acid residue positions corresponding to (i) residues 13-28; (ii) residues 38-42; and (iii) residues 122-123 of SEQ ID NO:32, respectively.

157. An isolated polypeptide according to any of paragraphs 17-21, which comprises the following discontinuous amino acid sequences:

(i)
(SEQ ID NO: 71)
YNSTSFSTFKCYGVSPTKLNDLCFT;
(ii)
(SEQ ID NO: 72)
DDFT
(iii)
(SEQ ID NO: 62)
FELLN

158. A polypeptide according to paragraph 157, wherein the discontinuous amino acid sequences (i), (ii), and (iii) are at amino acid residue positions corresponding to (i) residues 51-75; (ii) residues 109-112; and (iii) residues 197-201 of SEQ ID NO:28, respectively.

159. An isolated polypeptide according to any of paragraphs 27-29, which comprises the following discontinuous amino acid sequences:

(i)
(SEQ ID NO: 71)
YNSTSFSTFKCYGVSPTKLNDLCFT;
(ii)
(SEQ ID NO: 72)
DDFT
(iii)
(SEQ ID NO: 62)
FELLN

160. A polypeptide according to paragraph 159, wherein the discontinuous amino acid sequences (i), (ii), and (iii) are at amino acid residue positions corresponding to (i) residues 51-75; (ii) residues 109-112; and (iii) residues 197-201 of SEQ ID NO:30, respectively.

161. An isolated polypeptide according to any of paragraphs 30-35, which comprises the following discontinuous amino acid sequences:

(i)
(SEQ ID NO: 71)
YNSTSFSTFKCYGVSPTKLNDLCFT;
(ii)
(SEQ ID NO: 72)
DDFT
(iii)
(SEQ ID NO: 62)
FELLN

162. A polypeptide according to paragraph 161, wherein the discontinuous amino acid sequences (i), (ii), and (iii) are at amino acid residue positions corresponding to (i) residues 51-75; (ii) residues 109-112; and (iii) residues 197-201 of SEQ ID NO:31, respectively.

163. An isolated polypeptide according to any of paragraphs 17-21, which comprises the following discontinuous amino acid sequences:

(i)
(SEQ ID NO: 63)
RGDEVRQ;
(ii)
(SEQ ID NO: 73)
TGVIADY;
(iii)
(SEQ ID NO: 74)
YRSLRKSK;
(iv)
(SEQ ID NO: 75)
YSPGGK
(v)
(SEQ ID NO: 76)
FNCYYPLRSYGFFPTNGVGY

164. A polypeptide according to paragraph 163, wherein the discontinuous amino acid sequences (i), (ii), and (iii) are at amino acid residue positions corresponding to (i) residues 51-75; (ii) residues 109-112; and (iii) residues 197-201 of SEQ ID NO:28, respectively.

165. An isolated polypeptide according to any of paragraphs 22-26, which comprises the following discontinuous amino acid sequences:

(i)
(SEQ ID NO: 63)
RGDEVRQ;
(ii)
(SEQ ID NO: 73)
TGVIADY;
(iii)
(SEQ ID NO: 74)
YRSLRKSK;
(iv)
(SEQ ID NO: 75)
YSPGGK
(v)
(SEQ ID NO: 76)
FNCYYPLRSYGFFPTNGVGY

166. A polypeptide according to paragraph 165, wherein the discontinuous amino acid sequences (i), (ii), and (iii) are at amino acid residue positions corresponding to (i) residues 51-75; (ii) residues 109-112; and (iii) residues 197-201 of SEQ ID NO:29, respectively.

167. An isolated polypeptide according to any of paragraphs 30-35, which comprises the following discontinuous amino acid sequences:

(i)
(SEQ ID NO: 63)
RGDEVRQ;
(ii)
(SEQ ID NO: 73)
TGVIADY;
(iii)
(SEQ ID NO: 74)
YRSLRKSK;
(iv)
(SEQ ID NO: 75)
YSPGGK
(v)
(SEQ ID NO: 77)
FNCYYPLRSYGFFPTNGTGY

168. A polypeptide according to paragraph 167, wherein the discontinuous amino acid sequences (i), (ii), and (iii) are at amino acid residue positions corresponding to (i) residues 51-75; (ii) residues 109-112; and (iii) residues 197-201 of SEQ ID NO:31, respectively.

169. An isolated polypeptide according to any of paragraphs 36-41, which comprises the following discontinuous amino acid sequences:

(i)
(SEQ ID NO: 63)
RGDEVRQ;
(ii)
(SEQ ID NO: 73)
TGVIADY;
(iii)
(SEQ ID NO: 74)
YRSLRKSK;
(iv)
(SEQ ID NO: 75)
YSPGGK
(v)
(SEQ ID NO: 77)
FNCYYPLRSYGFFPTNGTGY

170. A polypeptide according to paragraph 169, wherein the discontinuous amino acid sequences (i), (ii), and (iii) are at amino acid residue positions corresponding to (i) residues 51-75; (ii) residues 109-112; and (iii) residues 197-201 of SEQ ID NO:32, respectively.

171. An isolated polypeptide comprising an amino acid sequence with the following discontinuous amino acid sequences:

(i)
(SEQ ID NO: 57)
NITNLCPFGEVFNATK;
(ii)
(SEQ ID NO: 58)
KKISN;
(iii)
(SEQ ID NO: 59)
NI.

172. An isolated polypeptide comprising an amino acid sequence with the following discontinuous amino acid sequences:

(i)
(SEQ ID NO: 60)
YNSTFFSTFKCYGVSPTKLN DLCFS;
(ii)
(SEQ ID NO: 61)
DDFM;
(iii)
(SEQ ID NO: 62)
FELLN.

173. An isolated polypeptide comprising an amino acid sequence with the following discontinuous amino acid sequences:

(i)
(SEQ ID NO: 63)
RGDEVRQ;
(ii)
(SEQ ID NO: 64)
TGKIADY;
(iii)
(SEQ ID NO: 65)
YRLFRKSN;
(iv)
(SEQ ID NO: 66)
YQAGST;
(v)
(SEQ ID NO: 67)
FNCYFPLQSYGFQPTNGVGY.

174. An isolated polypeptide comprising an amino acid sequence with the following discontinuous amino acid sequences:

(i)
(SEQ ID NO: 68)
NITNLCPFGEVFNATR;
(ii)
(SEQ ID NO: 69)
KRISN;
(iii)
(SEQ ID NO: 70)
NL

175. An isolated polypeptide comprising an amino acid sequence with the following discontinuous amino acid sequences:

(i)
(SEQ ID NO: 71)
YNSTSFSTFKCYGVSPTKLNDLCFT;
(ii)
(SEQ ID NO: 72)
DDFT
(iii)
(SEQ ID NO: 62)
FELLN

176. An isolated polypeptide comprising an amino acid sequence with the following discontinuous amino acid sequences:

(i)
(SEQ ID NO: 63)
RGDEVRQ;
(ii)
(SEQ ID NO: 73)
TGVIADY;
(iii)
(SEQ ID NO: 74)
YRSLRKSK;
(iv)
(SEQ ID NO: 75)
YSPGGK
(v)
(SEQ ID NO: 76)
FNCYYPLRSYGFFPTNGVGY

177. An isolated polypeptide comprising an amino acid sequence with the following discontinuous amino acid sequences:

(i)
(SEQ ID NO: 63)
RGDEVRQ;
(ii)
(SEQ ID NO: 73)
TGVIADY;
(iii)
(SEQ ID NO: 74)
YRSLRKSK;
(iv)
(SEQ ID NO: 75)
YSPGGK
(v)
(SEQ ID NO: 77)
FNCYYPLRSYGFFPTNGTGY

178. A polypeptide according to any of paragraphs 141-177, wherein the discontinuous amino acid sequences are present in the order recited.

179. A polypeptide according to any of paragraphs 141-178, wherein each discontinuous amino acid sequence is separated by at least 3 amino acid residues from an adjacent discontinuous amino acid sequence.

180. A polypeptide according to any of paragraphs 141-179, wherein each discontinuous amino acid sequence is separated by upto 100 amino acid residues from an adjacent discontinuous amino acid sequence.

181. A polypeptide according to any of paragraphs 141-180, which is up to 250, 500, 750, 1,000, 1,250, or 1,500 amino acid residues in length.

182. An isolated nucleic acid molecule encoding a polypeptide according to any of paragraphs 1 to 181, or the complement thereof.

183. An isolated nucleic acid molecule according to paragraph 182, comprising a nucleotide sequence of SEQ ID NO: 18, 16, or 14, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with a nucleotide sequence of SEQ ID NO: 18, 16, or 14 over its entire length, or the complement thereof.

184. An isolated nucleic acid molecule according to paragraph 182, comprising a nucleotide sequence of SEQ ID NO:37, 38, 39, or 40, or the complement thereof.

185. An isolated nucleic acid molecule comprising a nucleotide sequence encoding a SARS2 truncated S protein of amino acid sequence SEQ ID NO:9 (COV_T2_3), or the complement thereof.

186. A nucleic acid molecule according to paragraph 185, which comprises a nucleotide sequence of SEQ ID NO: 10, or the complement thereof.

187. An isolated nucleic acid molecule comprising a nucleotide sequence encoding a SARS2 S protein RBD of amino acid sequence SEQ ID NO:11 (COV_T2_6), or the complement thereof.

188. A nucleic acid molecule according to paragraph 187, which comprises a nucleotide sequence of SEQ ID NO: 12, or the complement thereof.

189. A vector comprising a nucleic acid molecule of any of paragraphs 182 to 188.

190. A vector according to paragraph 189, comprising a nucleic acid molecule encoding a polypeptide according to any of paragraphs 1 to 61, or 118 to 181.

191. A vector according to paragraph 189 or 190, comprising a nucleic acid molecule encoding a polypeptide according to any of paragraphs 62 to 74.

192. A vector according to any of paragraphs 189 to 191, comprising a nucleic acid molecule encoding a polypeptide according to any of paragraphs 75 to 101.

193. A vector according to any of paragraphs 189 to 192, comprising a nucleic acid molecule encoding a polypeptide according to any of paragraphs 102 to 117.

194. A vector according to paragraph 189, which further comprises a promoter operably linked to the nucleic acid.

195. A vector according to any of paragraphs 190 to 194, which further comprises, for each nucleic acid molecule of the vector encoding a polypeptide, a separate promoter operably linked to that nucleic acid molecule.

196. A vector according to paragraph 194, wherein the promoter is for expression of a polypeptide encoded by the nucleic acid in mammalian cells.

197. A vector according to paragraph 195, wherein the, or each promoter is for expression of a polypeptide encoded by the nucleic acid molecule in mammalian cells.

198. A vector according to paragraph 194, wherein the promoter is for expression of a polypeptide encoded by the nucleic acid in yeast or insect cells.

199. A vector according to paragraph 195, wherein the, or each promoter is for expression of a polypeptide encoded by the nucleic acid molecule in yeast or insect cells.

200. A vector according to any of paragraphs 189 to 199, which is a vaccine vector.

201. A vector according to paragraph 200, which is a viral vaccine vector, a bacterial vaccine vector, an RNA vaccine vector, or a DNA vaccine vector.

202. A vector according to paragraph 200, which is an mRNA vaccine vector.

203. An isolated cell comprising a vector of any of paragraphs 189 to 202.

204. A fusion protein comprising a polypeptide according to any of paragraphs 1 to 181.

205. A pharmaceutical composition comprising a polypeptide according to any of paragraphs 1 to 181, and a pharmaceutically acceptable carrier, excipient, or diluent.

206. A pharmaceutical composition according to paragraph 205, comprising a polypeptide according to any of paragraphs 1 to 61, or 118 to 181.

207. A pharmaceutical composition according to paragraph 205 or 206, comprising a polypeptide according to any of paragraphs 62 to 74.

208. A pharmaceutical composition according to any of paragraphs 205 to 207, comprising a polypeptide according to any of paragraphs 75 to 101.

209. A pharmaceutical composition according to any of paragraphs 205 to 208, comprising a polypeptide according to any of paragraphs 102 to 117.

210. A pharmaceutical composition comprising a nucleic acid according to any of paragraphs 182 to 188, and a pharmaceutically acceptable carrier, excipient, or diluent.

211. A pharmaceutical composition according to paragraph 210, comprising a nucleic acid molecule encoding a polypeptide according to any of paragraphs 1 to 61, or 118 to 181.

212. A pharmaceutical composition according to paragraph 210 or 211, comprising a nucleic acid molecule encoding a polypeptide according to any of paragraphs 62 to 74.

213. A pharmaceutical composition according to any of paragraphs 210 to 212, comprising a nucleic acid molecule encoding a polypeptide according to any of paragraphs 75 to 101.

214. A pharmaceutical composition according to any of paragraphs 210 to 213, comprising a nucleic acid molecule encoding a polypeptide according to any of paragraphs 102 to 117.

215. A pharmaceutical composition comprising a vector according to any of paragraphs 189 to 202, and a pharmaceutically acceptable carrier, excipient, or diluent.

216. A pharmaceutical composition according to any of paragraphs 205 to 215, which further comprises an adjuvant for enhancing an immune response in a subject to the polypeptide, or to a polypeptide encoded by the nucleic acid, of the composition.

217. A combined preparation, which comprises:
i) a polypeptide according to any of paragraphs 1 to 61, or 118 to 181; and
ii) a polypeptide according to any of paragraphs 62 to 74.

218. A combined preparation, which comprises:
i) a polypeptide according to any of paragraphs 1 to 61, or 118 to 181; and
ii) a polypeptide according to any of paragraphs 75 to 101.

219. A combined preparation, which comprises:
i) a polypeptide according to any of paragraphs 62 to 74; and
ii) a polypeptide according to any of paragraphs 75 to 101.

220. A combined preparation, which comprises:
i) a polypeptide according to any of paragraphs 1 to 61, or 118 to 181; and
ii) a polypeptide according to any of paragraphs 102 to 117.

221. A combined preparation, which comprises:
i) a polypeptide according to any of paragraphs 62 to 74; and
ii) a polypeptide according to any of paragraphs 102 to 117.

222. A combined preparation, which comprises:
i) a polypeptide according to any of paragraphs 75 to 101; and
ii) a polypeptide according to any of paragraphs 102 to 117.

223. A combined preparation, which comprises:
i) a polypeptide according to any of paragraphs 1 to 61, or 118 to 181;
ii) a polypeptide according to any of paragraphs 62 to 74; and
iii) a polypeptide according to any of paragraphs 75 to 101.

224. A combined preparation, which comprises:
i) a polypeptide according to any of paragraphs 62 to 74;
ii) a polypeptide according to any of paragraphs 75 to 101; and
iii) a polypeptide according to any of paragraphs 102 to 117.

225. A combined preparation, which comprises:
i) a polypeptide according to any of paragraphs 1 to 61, or 118 to 181;
ii) a polypeptide according to any of paragraphs 62 to 74; and
iii) a polypeptide according to any of paragraphs 102 to 117.

226. A combined preparation, which comprises:
i) a polypeptide according to any of paragraphs 1 to 61, or 118 to 181;
ii) a polypeptide according to any of paragraphs 75 to 101; and
iii) a polypeptide according to any of paragraphs 102 to 117.

227. A combined preparation, which comprises:
i) a polypeptide according to any of paragraphs 1 to 61, or 118 to 181;
ii) a polypeptide according to any of paragraphs 62 to 74;
iii) a polypeptide according to any of paragraphs 75 to 101; and
iv) a polypeptide according to any of paragraphs 102 to 117.

228. A combined preparation, which comprises:
i) a nucleic acid encoding a polypeptide according to any of paragraphs 1 to 61, or 118 to 181; and
ii) a nucleic acid encoding a polypeptide according to any of paragraphs 62 to 74.

229. A combined preparation, which comprises:
i) a nucleic acid encoding a polypeptide according to any of paragraphs 1 to 61, or 118 to 181; and
ii) a nucleic acid encoding a polypeptide according to any of paragraphs 75 to 101.

230. A combined preparation, which comprises:
i) a nucleic acid encoding a polypeptide according to any of paragraphs 62 to 74; and
ii) a nucleic acid encoding a polypeptide according to any of paragraphs 75 to 101.

231. A combined preparation, which comprises:
i) a nucleic acid encoding a polypeptide according to any of paragraphs 1 to 61, or 118 to 181; and
ii) a nucleic acid encoding a polypeptide according to any of paragraphs 102 to 117.

232. A combined preparation, which comprises:
i) a nucleic acid encoding a polypeptide according to any of paragraphs 62 to 74; and
ii) a nucleic acid encoding a polypeptide according to any of paragraphs 102 to 117.

233. A combined preparation, which comprises:
i) a nucleic acid encoding a polypeptide according to any of paragraphs 75 to 101;
and
ii) a nucleic acid encoding a polypeptide according to any of paragraphs 102 to 117.

234. A combined preparation, which comprises:
i) a nucleic acid encoding a polypeptide according to any of paragraphs 1 to 61, or 118 to 181;
ii) a nucleic acid encoding a polypeptide according to any of paragraphs 62 to 74; and
iii) a nucleic acid encoding a polypeptide according to any of paragraphs 75 to 101.

235. A combined preparation, which comprises:
i) a nucleic acid encoding a polypeptide according to any of paragraphs 62 to 74;
ii) a nucleic acid encoding a polypeptide according to any of paragraphs 75 to 101; and
iii) a nucleic acid encoding a polypeptide according to any of paragraphs 102 to 117.

236. A combined preparation, which comprises:
i) a nucleic acid encoding a polypeptide according to any of paragraphs 1 to 61, or 118 to 181;
ii) a nucleic acid encoding a polypeptide according to any of paragraphs 62 to 74; and
iii) a nucleic acid encoding a polypeptide according to any of paragraphs 102 to 117. 237. A combined preparation, which comprises:

i) a nucleic acid encoding a polypeptide according to any of paragraphs 1 to 61, or 118 to 181;
ii) a nucleic acid encoding a polypeptide according to any of paragraphs 75 to 101; and
iii) a nucleic acid encoding a polypeptide according to any of paragraphs 102 to 117.

238. A combined preparation, which comprises:
i) a nucleic acid encoding a polypeptide according to any of paragraphs 1 to 61, or 118 to 181;
ii) a nucleic acid encoding a polypeptide according to any of paragraphs 62 to 74;
iii) a nucleic acid encoding a polypeptide according to any of paragraphs 75 to 101; and
iv) a nucleic acid encoding a polypeptide according to any of paragraphs 102 to 117.

239. A pharmaceutical composition according to any of paragraphs 211 to 214, wherein the or each nucleic acid molecule is provided by a vector.

240. A combined preparation according to any of paragraphs to 228 to 238, wherein each nucleic acid is provided by a vector.

241. A pharmaceutical composition according to paragraph 239, or a combined preparation according to paragraph 240, wherein the, or each vector is a vaccine vector.

242. A pharmaceutical composition according to paragraph 239 or 241, or a combined preparation according to paragraph 240 or 241, wherein the, or each vaccine vector is a viral vaccine vector, a bacterial vaccine vector, an RNA vaccine vector, an mRNA vaccine vector, or a DNA vaccine vector.

243. A pharmaceutical composition or a combined preparation according to paragraph 242, wherein the, or each vaccine vector is a DNA vaccine vector.

244. A pharmaceutical composition or a combined preparation according to paragraph 242, wherein the, or each vaccine vector is an mRNA vaccine vector.

245. A nucleic acid according to any of paragraphs 182-188, which comprises one or more modified nucleosides.

246. A vector according to any of paragraphs 189-202, wherein the nucleic acid of the vector comprises one or more modified nucleosides.

247. A pharmaceutical composition according to any of paragraphs 210-216, 239, or 241-244, wherein the or each nucleic acid of the composition comprises one or more modified nucleosides.

248. A combined preparation according to any of paragraphs 228-238, or 240-244, wherein each nucleic acid of the combined preparation comprises one or more modified nucleosides.

249. A nucleic acid according to paragraph 245, a vector according to paragraph 246, a pharmaceutical composition according to paragraph 247, or a combined preparation according to paragraph 248, wherein the or each nucleic acid comprises a messenger RNA (mRNA).

250. A nucleic acid according to paragraph 245 or 249, a vector according to paragraph 246 or 249, a pharmaceutical composition according to paragraph 247 or 249, or a combined preparation according to paragraph 248 or 249, wherein the one or more modified nucleosides comprise a 1-methylpseudouridine modification.

251. A nucleic acid according to paragraph 245 or 249 or 250, a vector according to paragraph 246 or 249 or 250, a pharmaceutical composition according to paragraph 247 or 249 or 250, or a combined preparation according to paragraph 248 or 249 or 250, wherein the one or more modified nucleosides comprise a 1-methylpseudouridine modification.

252. A nucleic acid according to any of paragraphs 245, or 249-251, a vector according to any of paragraphs 246, or 249-251, a pharmaceutical composition according to any of paragraphs 247, or 249-251, or a combined preparation according to any of paragraphs 248-251, wherein at least 80% of the uridines in the open reading frame have been modified.

253. A pseudotyped virus comprising a polypeptide according to any of paragraphs 1 to 181.

254. A method of inducing an immune response to a coronavirus in a subject, which comprises administering to the subject an effective amount of a polypeptide according to any of paragraphs 1 to 181, a nucleic acid according to any of paragraphs 182 to 188, 245, or 249-252, a vector according to any of paragraphs 189 to 202, 246, or 249-252, a pharmaceutical composition according to any of paragraphs 205 to 216, 239, 241-244, 247, or 249-252, or a combined preparation according to any of paragraphs 217-238, 240-244, or 248-252.

255. A method of immunising a subject against a coronavirus, which comprises administering to the subject an effective amount of a polypeptide according to any of paragraphs 1 to 181, a nucleic acid according to any of paragraphs 182 to 188, 245, or 249-252, a vector according to any of paragraphs 189 to 202, 246, or 249-252, a pharmaceutical composition according to any of paragraphs 205 to 216, 239, 241-244, 247, or 249-252, or a combined preparation according to any of paragraphs 217-238, 240-244, or 248-252.

256. A polypeptide according to any of paragraphs 1 to 181, a nucleic acid according to any of paragraphs 182 to 188, 245, or 249-252, a vector according to any of paragraphs 189 to 202, 246, or 249-252, a pharmaceutical composition according to any of paragraphs 205 to 216, 239, 241-244, 247, or 249-252, or a combined preparation according to any of paragraphs 217-238, 240-244, or 248-252, for use as a medicament.

257. A polypeptide according to any of paragraphs 1 to 181, a nucleic acid according to any of paragraphs 182 to 188, 245, or 249-252, a vector according to any of paragraphs 189 to 202, 246, or 249-252, a pharmaceutical composition according to any of paragraphs 205 to 216, 239, 241-244, 247, or 249-252, or a combined preparation according to any of paragraphs 217-238, 240-244, or 248-252, for use in the prevention, treatment, or amelioration of a coronavirus infection.

258. Use of a polypeptide according to any of paragraphs 1 to 181, a nucleic acid according to any of paragraphs 182 to 188, 245, or 249-252, a vector according to any of paragraphs 189 to 202, 246, or 249-252, a pharmaceutical composition according to any of paragraphs 205 to 216, 239, 241-244, 247, or 249-252, or a combined preparation according to any of paragraphs 217-238, 240-244, or 248-252, in the manufacture of a medicament for the prevention, treatment, or amelioration of a coronavirus infection.

259. A method according to paragraph 254 or 255, a polypeptide, nucleic acid, vector, pharmaceutical composition, or combined preparation, for use according to paragraph 257, or use according to paragraph 258, wherein the coronavirus is a β-coronavirus.

260. A method, or a polypeptide, nucleic acid, vector, pharmaceutical composition, or combined preparation for use, or use according to paragraph 259, wherein the β-coronavirus is a lineage B or C β-coronavirus.

261. A method, or a polypeptide, nucleic acid, vector, or pharmaceutical composition for use, or use according to paragraph 259, wherein the β-coronavirus is a lineage B β-coronavirus.

262. A method, or a polypeptide, nucleic acid, vector, or pharmaceutical composition for use, or use according to paragraph 260 or 261, wherein the lineage B β-coronavirus is SARS-CoV or SARS-CoV-2.

263. A method, or a polypeptide, nucleic acid, vector, or pharmaceutical composition for use, or use according to 260, wherein the lineage C β-coronavirus is MERS-COV.

264. A method, or a polypeptide, nucleic acid, vector, pharmaceutical composition, or combined preparation for use, or use according to paragraph 259, wherein the beta-coronavirus is a variant of concern (VOC).

265. A method, or a polypeptide, nucleic acid, vector, pharmaceutical composition, or combined preparation for use, or use according to paragraph 259, wherein the beta-coronavirus is a SARS-CoV-2 VOC.

266. A method, or a polypeptide, nucleic acid, vector, pharmaceutical composition, or combined preparation for use, or use according to paragraph 259, wherein the beta-coronavirus is a SARS-CoV-2 beta, gamma, or delta VOC.

Embodiments of the invention are now described, by way of example only, with reference to the accompanying drawings, in which:

FIG. 1 shows SARS S-protein architecture;

FIG. 2 shows a multiple sequence alignment of the S-protein (region around the S1 cleavage site) comparing SARS-CoV-1 isolate (SEQ ID NO:99) and closely related bat betacoronavirus isolate (SEQ ID NO: 100) with four SARS-CoV-2 isolates (SEQ ID NO: 101-104);

FIG. 3 shows a plasmid map for pEVAC DNA vector;

FIG. 4 shows Wuhan_Node1_RBD (CoV_T2_7) amino acid sequence (SEQ ID NO: 17) with amino acid residue differences highlighted in bold and underline from the respective alignments with AY274119_RBD (COV_T2_5) (SEQ ID NO:5) and EPI_ISL_402119_RBD (CoV_T2_6) (SEQ ID NO:11) amino acid sequences. Common differences from the two alignments are shown highlighted in grey. Amino acid insertions are shown boxed;

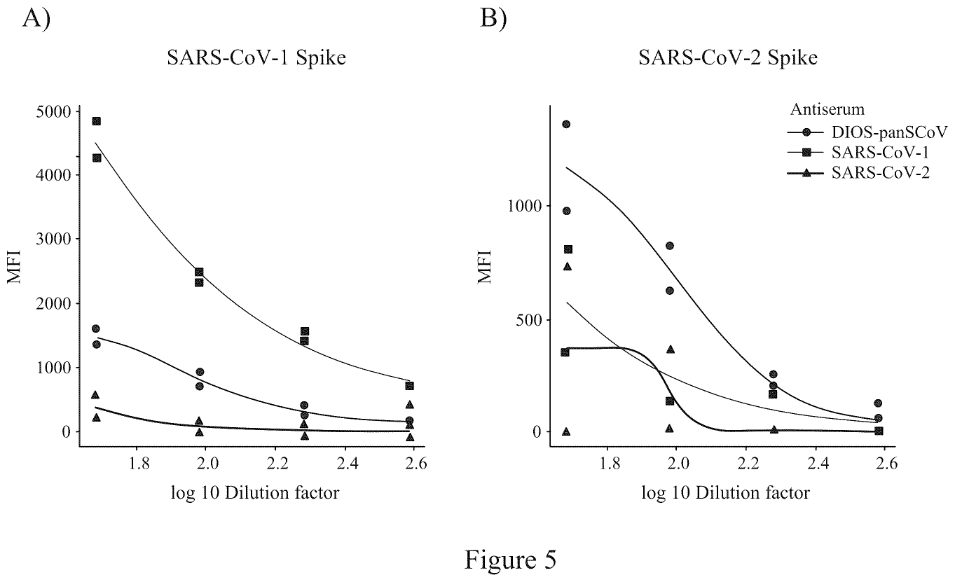

FIG. 5 shows dose response curves of antibody binding to full length Spike protein of SARS-CoV-1, or SARS-CoV-2 expressed on HEK293T cells. Flow cytometry based cell display assay reported in MFI (Median Fluorescent Intensity). In the left hand figure, the upper to lower curves are SARS-CoV-1, DIOS-panSCOV, SARS-CoV2; in the right hand figure, the upper to lower curves are DIOS-panSCoV, SARS-CoV-1, SARS-CoV2;

FIG. 6 shows coronavirus SARS Envelope protein sequence (SEQ ID NO:21), and its significant elements;

FIG. 7 shows a multiple sequence alignment of coronavirus Envelope protein sequences, comparing sequences for isolates of NL63 (SEQ ID NO: 106), 229E (SEQ ID NO:107), HKU1 (SEQ ID NOs: 108-109), MERS (SEQ ID NO:110), SARS (SEQ ID NO:21), and SARS2 (SEQ ID NO: 41), and consensus E protein sequences (SEQ ID NOs: 111-113);

FIG. 8 shows a multiple sequence alignment of coronavirus Membrane (M) protein sequences, comparing sequences for a SARS2 reference sequence (isolate NC_045512.2) against COV_M_T2_1 (Sarbeco_M_root) and COV_M_T2_2 (Sarbeco_M_Node88b_epitope_optimised);

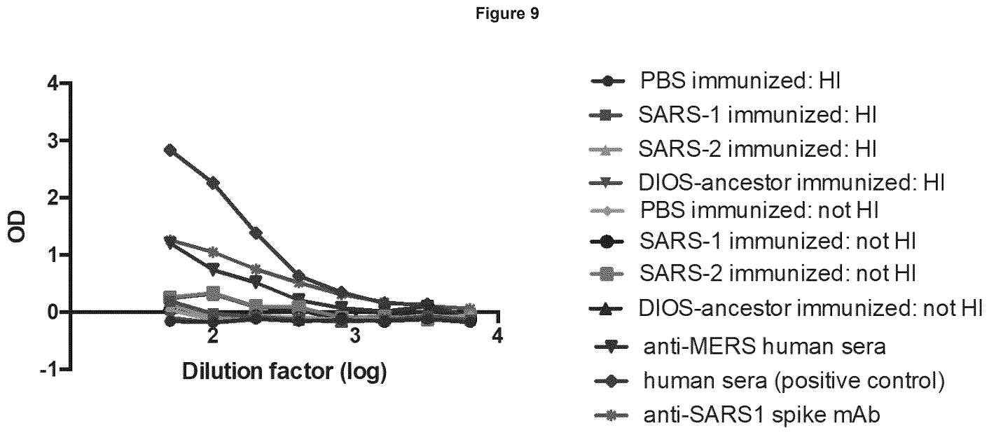

FIG. 9 shows binding (by ELISA) of mouse sera, collected following immunisation of mice with different full-length S protein genes, to SARS2 RBD;

FIG. 10 shows binding by FACS of mouse sera, collected following immunisation of mice with different DNA vaccines, to SARS1 spike protein and SARS2 spike protein;

FIG. 11 shows the ability of DNA vaccines encoding wild-type SARS1 or SARS2 spike protein (full-length, truncated, or RBD) to induce a neutralisation response to SARS1 and SARS2 pseudotypes—the only SARS2 immunogen which induces SARS2 pseudotype neutralising antibodies is the DNA encoding SARS2 RBD;

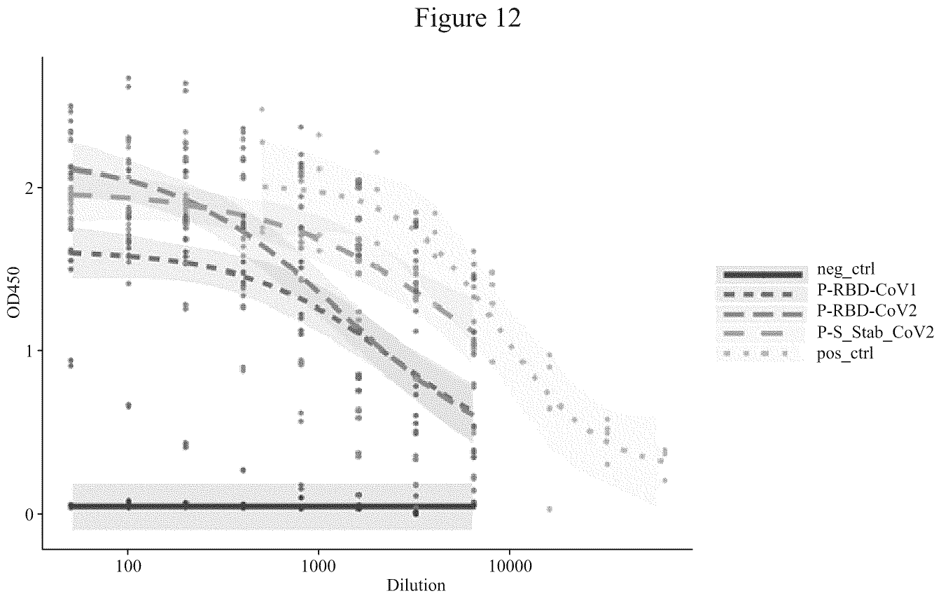

FIG. 12 shows the ability of SARS1 and SARS2 RBD protein vaccines to induce antibodies to SARS2 RBD;

FIG. 13 illustrates new RBD antigen designs based on the amino acid sequence of the RBD region (SEQ ID NO:119);

FIG. 14 shows the ability of different S protein RBD DNA vaccines to induce antibodies to SARS2 RBD-M7 DNA vaccine induces a stronger binding response (by ELISA) to SARS2 RBD than wild-type SARS2 RBD DNA vaccine (the uppermost curve, from the left hand end of the figure, is for SARS_2 RBD_mut1 (M7), the next curve down is for SARS_2 RBD);

FIG. 15 shows the results of a competition assay for inhibition of RBD-ACE2 interaction by sera collected following immunisation with M7 and wild-type SARS2 RBD DNA vaccines—the results show that M7 RBD DNA vaccine elicits a faster neutralisation response than wild-type RBD DNA vaccine;

FIG. 16 shows a SARS2 pseudotype neutralisation response induced by M7 and wild-type SARS2 RBD DNA vaccines: FIG. 16(a) bleed at week 2 from the immunised mice, FIG. 16(b) bleed at week 3 from the immunised mice, and FIG. 16(c) bleed at week 4 from the immunised mice-M7 is more neutralising in the early stages (the uppermost curve, from the left hand end of FIG. 16 (a), (b), (c), is for SARS2 RBD_mut1 (M7), the next curve down is for SARS_2 RBD);

FIG. 17 shows SARS2 pseudotype neutralisation IC₅₀ values for sera collected from the mice immunised with wild-type SARS2 RBD DNA vaccine, and M7 SARS2 RBD DNA vaccine. The dots in FIG. 17 show IC₅₀ values for individual mice, and the horizontal cross bars show the estimate based on all mice with 95% confidence intervals;

FIG. 18 shows that the supernatant of cells expressing M7 competes with other ACE2 binding viruses for ACE2 cell entry;

FIG. 19 shows the results of an ELISPOT assay showing T cell response to M7 SARS2 RBD DNA vaccine;

FIG. 20 shows an illustration of the M protein (SEQ ID NO: 114), and its significant elements;

FIG. 21 shows the spectra overlap (MALDI MS) of supernatants derived from HEK cells transfected with pEVAC plasmid encoding S protein RBD sequences;

FIG. 22 shows spectra for recombinant RBD proteins;

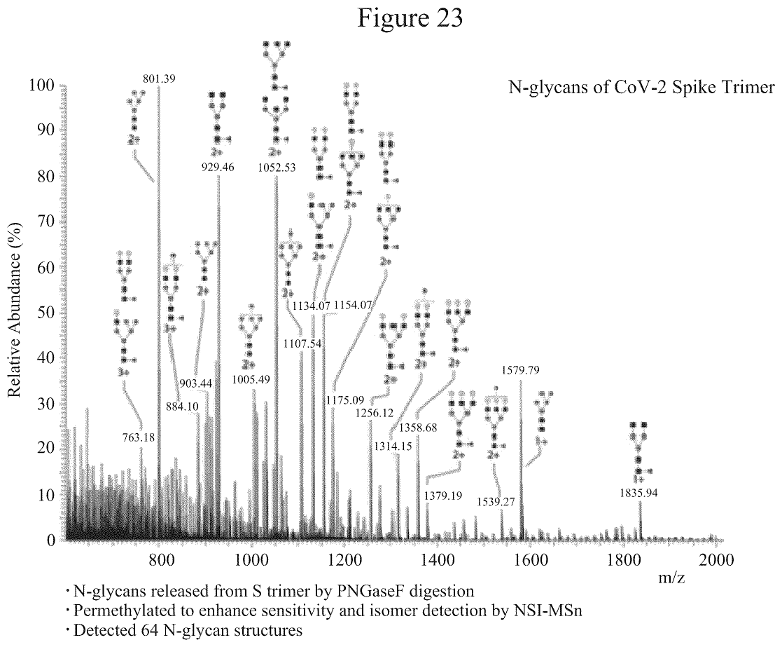

FIG. 23 provides a reference for glycosylation of the S protein;

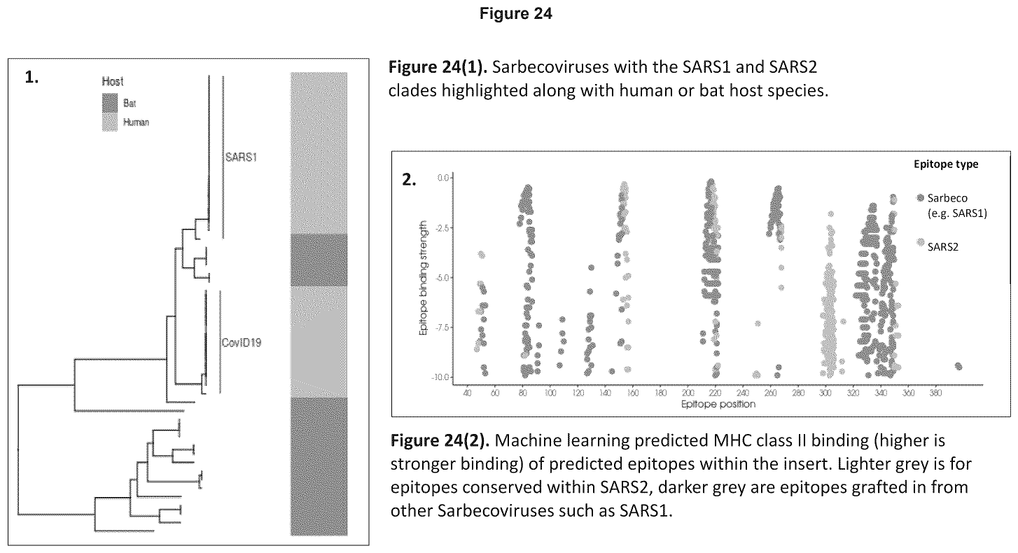

FIG. 24 shows coronavirus vaccine pan-Sarbecovirus vaccine coverage. Pan-Sarbecovirus protection: Beta-Coronaviruses including SARS-CoV-2 (SARS2), -1 (SARS1) & the many ACE2 receptor using Bat SARSr-COV that threaten to spillover into humans. Antigenic coverage achieved by universal Sarbecovirus β-cell and T-cell antigen targets: Part 1. Sarbecoviruses with the SARS1 and SARS2 clades highlighted along with human or bat host species. Part 2. Machine learning predicted MHC class II binding (higher is stronger binding) of predicted epitopes within the insert. Lighter grey is for epitopes conserved within SARS2, darker grey are epitopes grafted in from other Sarbecoviruses such as SARS1;

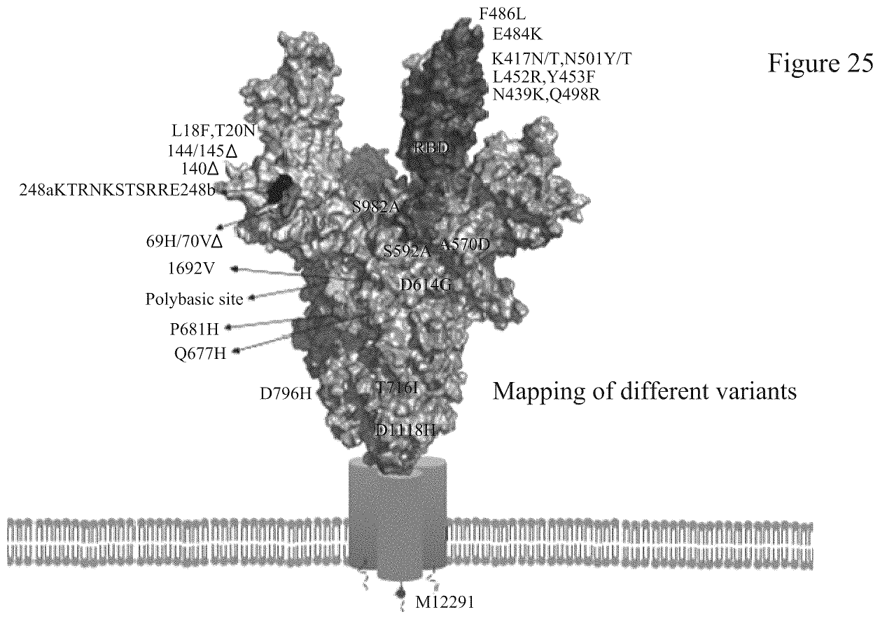

FIG. 25 illustrates mapping of different SARS-CoV-2 variants:

Inclusive list of all the important variants: Pink=exposed mutation; Black=insertion; Yellow=partially buried or fully buried; Purple=in the cytoplasmic tail; Blue colour=RBD; Wheat colour=NTD;

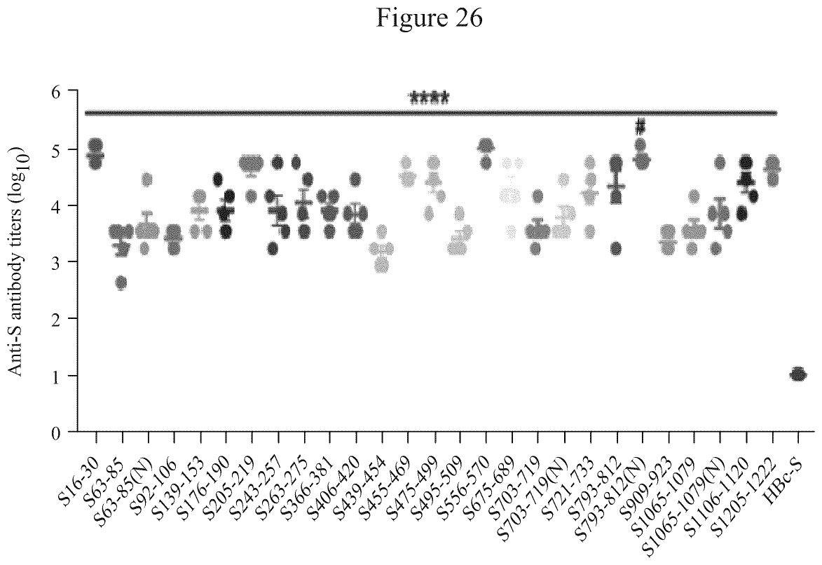

FIG. 26 shows the immunodominant and neutralization linear epitopes for SARS-CoV-2:

| EpitopesEpitopes | Variant | Immuno-dominant* |
|---|---|---|
| 16-30 | Japan | Yes |
| 92-106 | | |
| 139-153 | UK, Japan | |
| 243-257 | | |
| 406-420 | Japan, South Africa | |
| 439-454 | | |
| 455-499 | Japan, South Africa | Yes |
| 556-570 | UK | Yes |
| 675-689 | UK | |
| 721-733 | | Yes |

Study limited to Chinese population. Expressed peptides as VSV.
*Against G614 variant

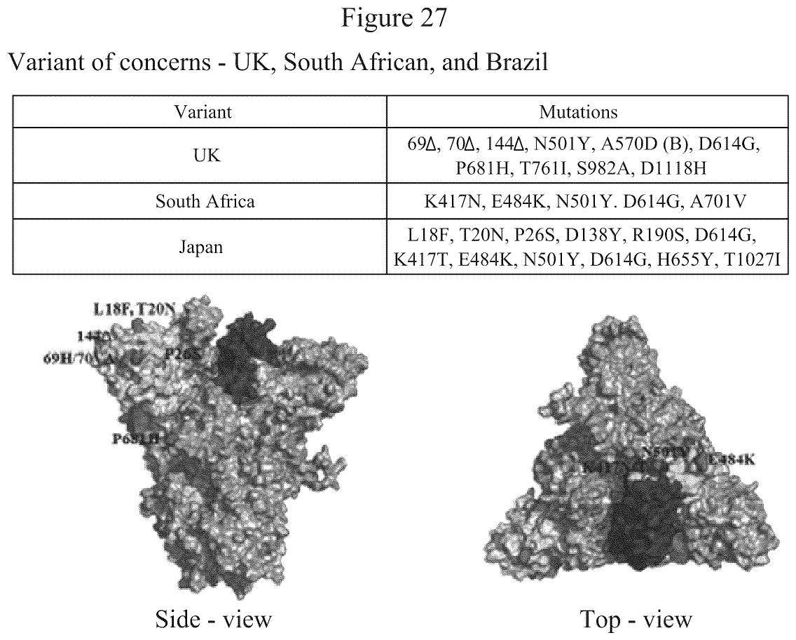

FIG. 27 contains a table describing the mutations in the variants of concern (UK, South African, and Brazil), and structural figures with immunodominant epitope coloured teal and mutations shown in red. RBD—Blue; NTD—wheat;

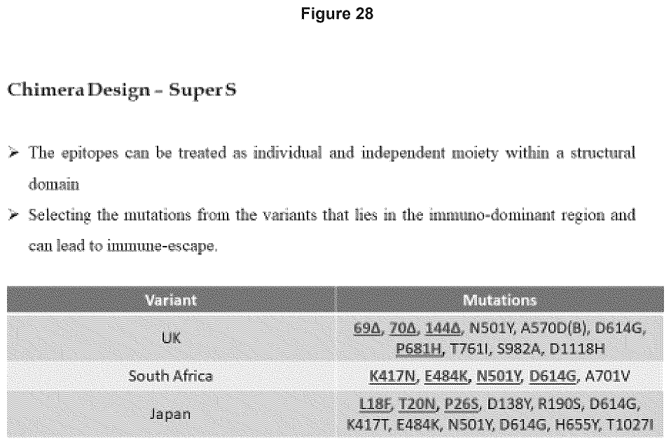

FIG. 28 explains the chimeric design of a super spike protein according to an embodiment of the invention;

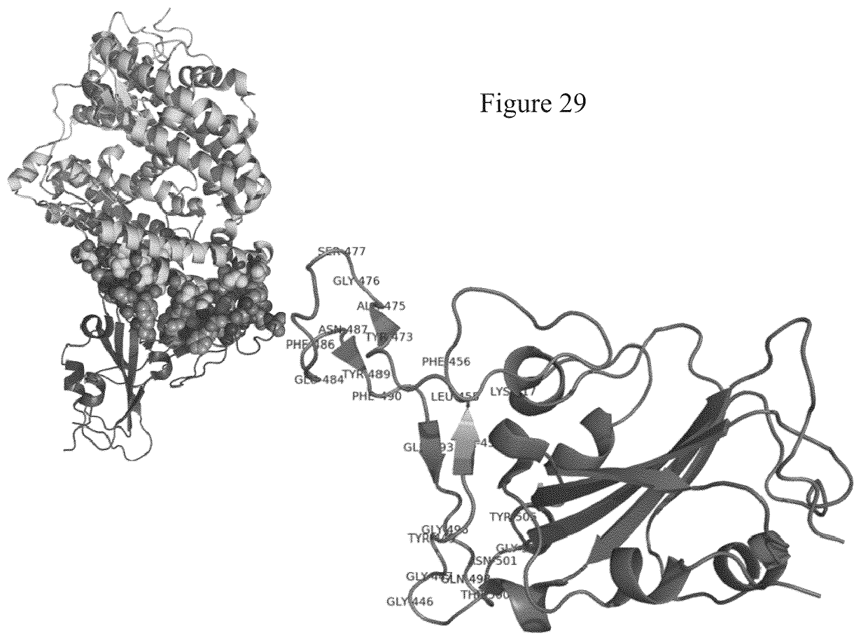

FIG. 29 illustrates the positions of the mutations on a structural image of the spike protein;

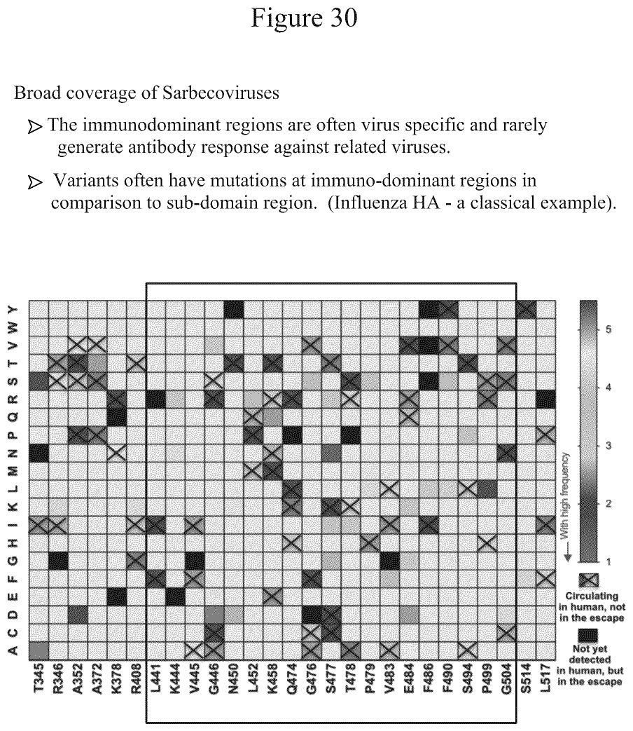

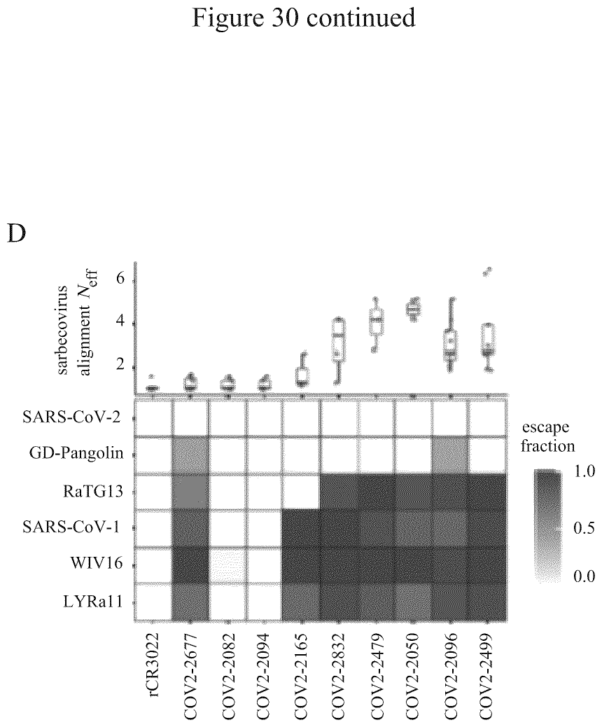

FIG. 30 shows data taken from the literature, showing maximum of current variants have mutation in RBM region and the other epitopes in RBD are conserved and the antibodies against them cross-react; Boxed is the RBM. Figure D—top is the distribution of entropy.

Lower the spread, better conserved in the represented sarbecoviruses. All the antibodies targeting this region show cross-neutralisation (white boxes). Black or grey boxes indicate no neutralisation;

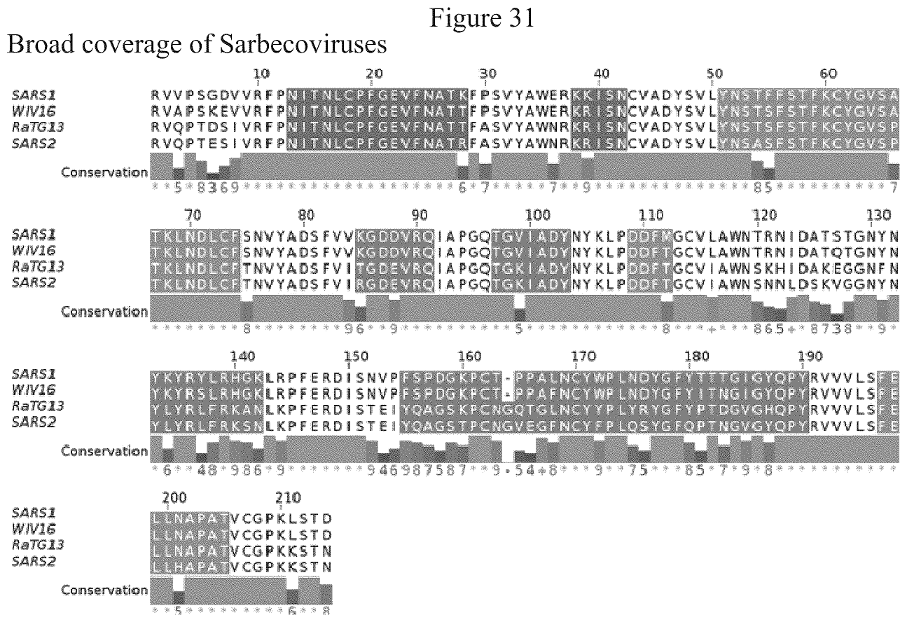

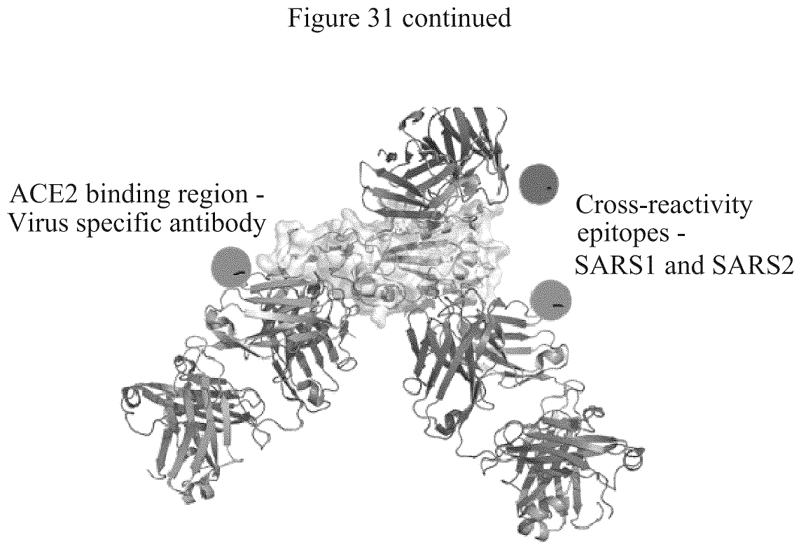

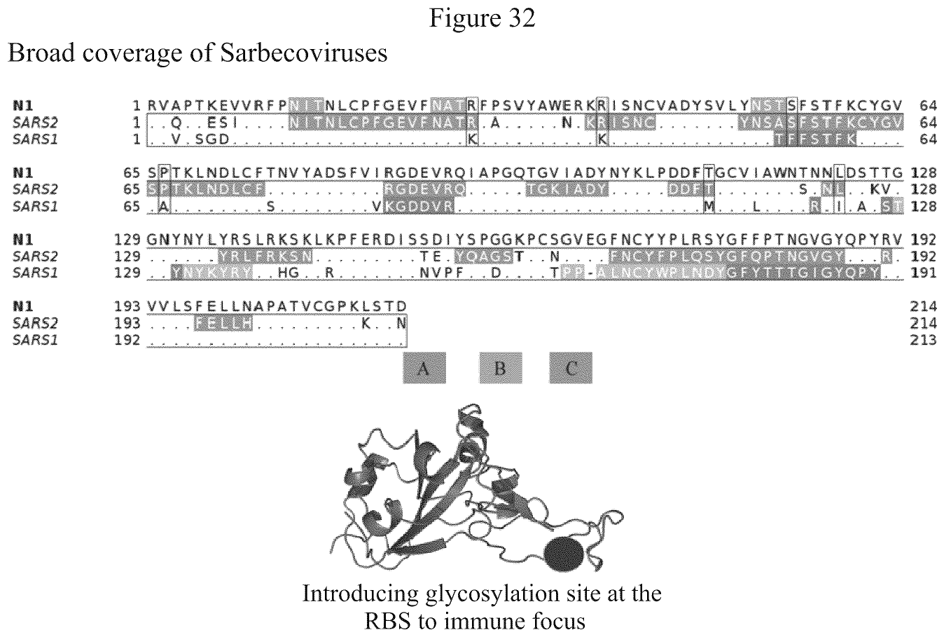

FIGS. 31 and 32 illustrate use of the structural information to identify epitopes, and to include this in the design of S proteins of the invention, and diverting the immune response by glycosylation. FIG. 31 shows RBD sequences of SARS1 (SEQ ID NO:5), WIV16 (SEQ ID NO: 102), RaTG13 (SEQ ID NO:116), and SARS2 (SEQ ID NO:11). In FIG. 32, N1-Phylogenetically optimised design (CoV_S_T2_13) (SEQ ID NO:27), SARS2 N1 (SEQ ID NO: 117), and SARS1 N1 (SEQ ID NO:118);

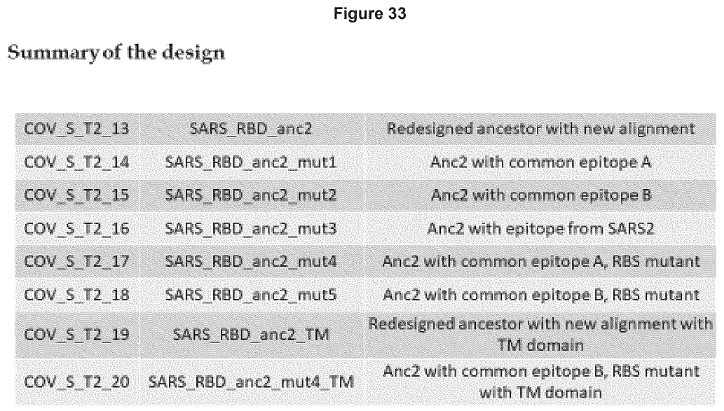

FIG. 33 summarises designs according to embodiments of the invention;

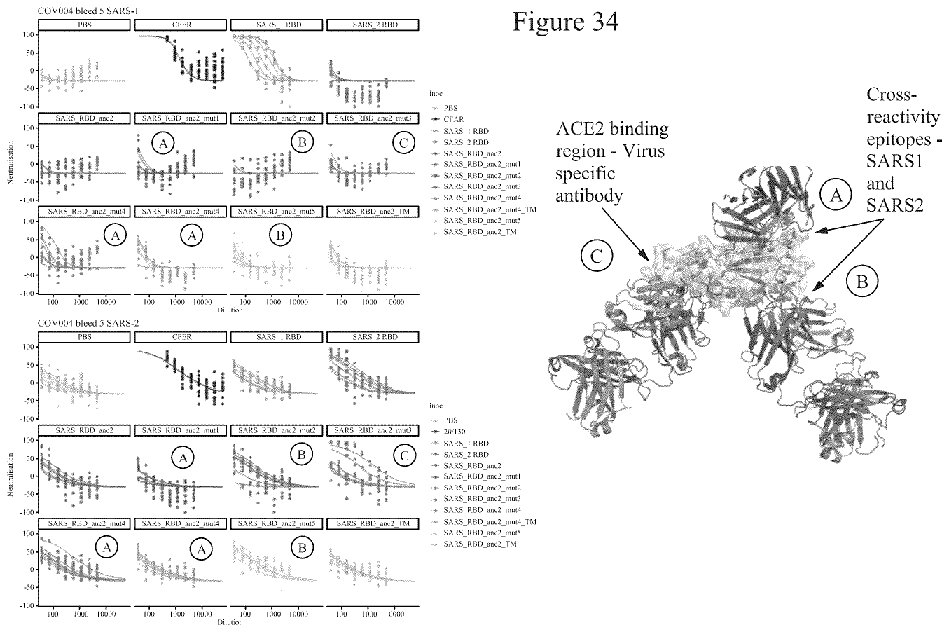

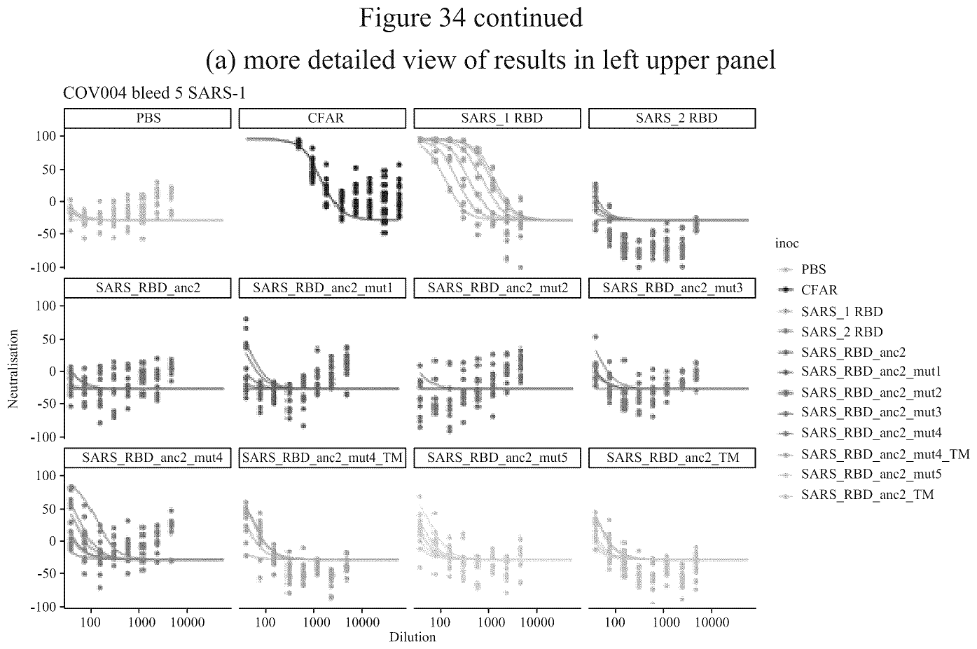

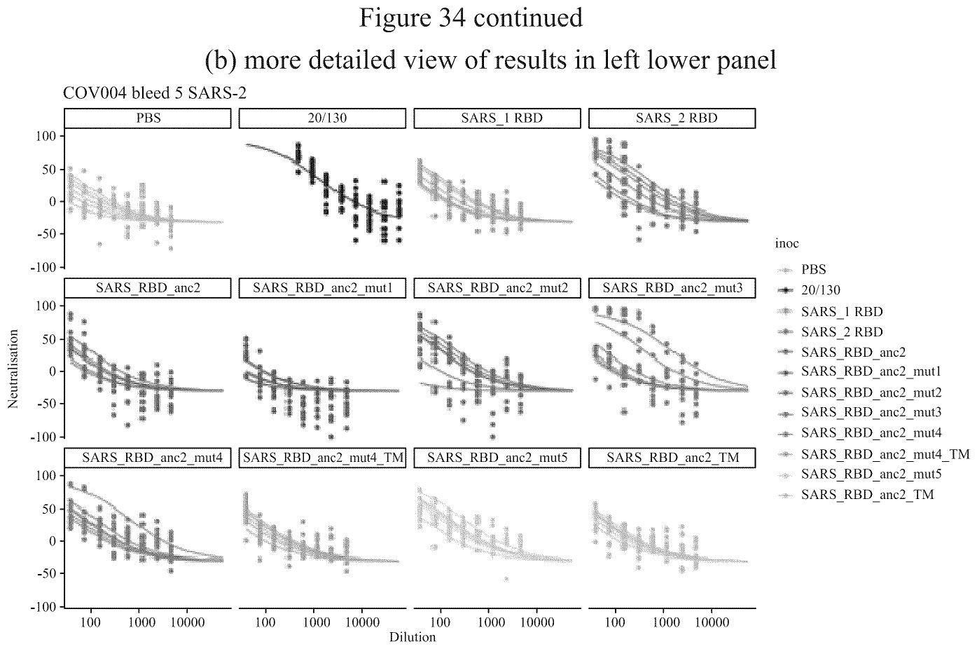

FIG. 34 summarises data obtained for designs according to embodiments of the invention;

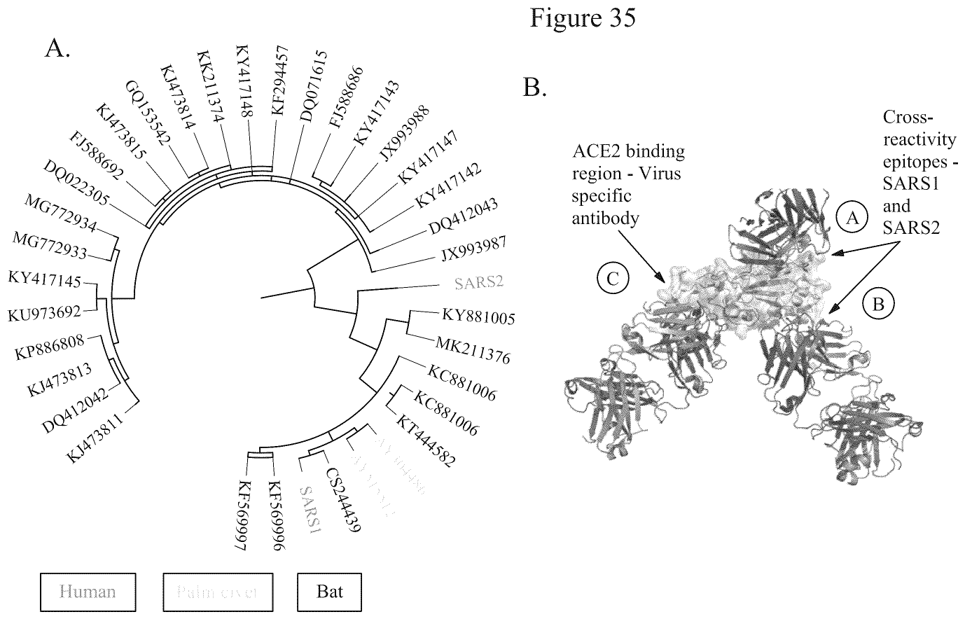

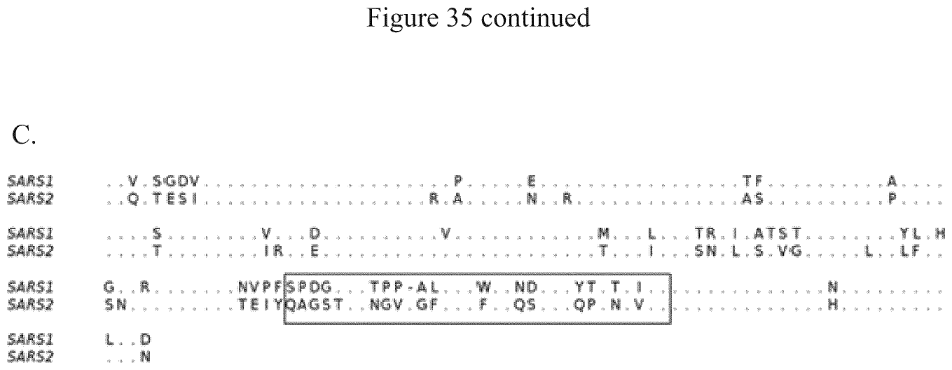

FIG. 35 In-silico design of a vaccine according to an embodiment of the invention:
A. Phylogenetic tree generated for sarbecoviruses using protein sequence of receptor binding domain (RBD) of the spike protein. The tree was generated using IQ-Tree. Human viruses are represented in green, palm civet viruses in pink and bat viruses in dark grey.
B. Structural model of the antibody-RBD complex. The antibodies are represented as cartoon and coloured green and orange and the RBD is represented as both cartoon and surface and coloured pink. The different epitope regions are labelled as A, B and C.
C. Sequence alignment of SARS-1 and SARS-2. Only the non-conserved amino acids are shown. The epitope C is boxed in black;

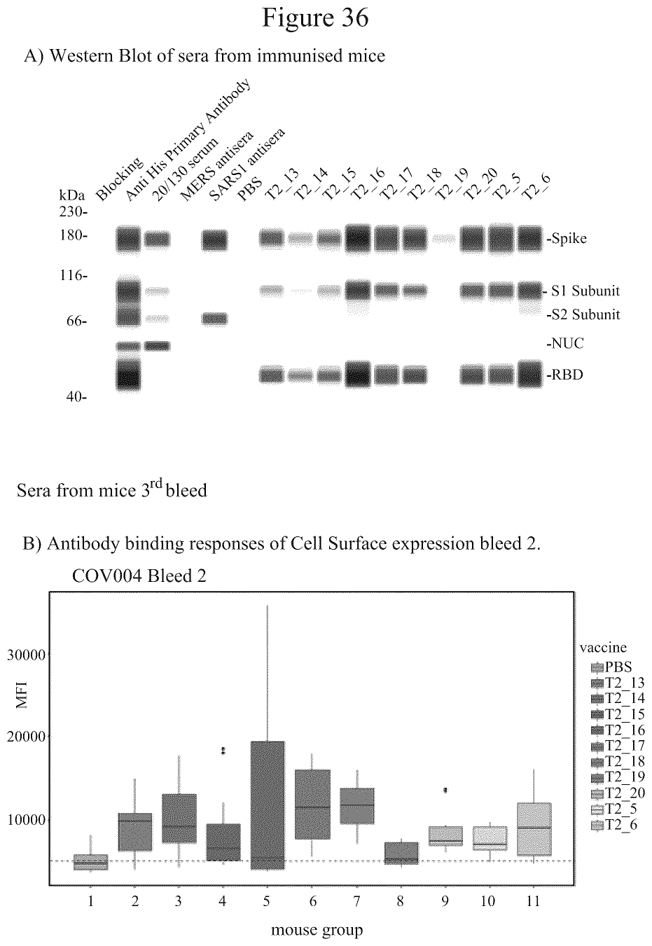

FIG. 36(A) shows a Western Blot of sera from mice immunised with the vaccine designs of Example 32 (COV_S_T2_13-20). FIG. 36 (B) shows antibody binding responses of Cell Surface expression bleed 2;

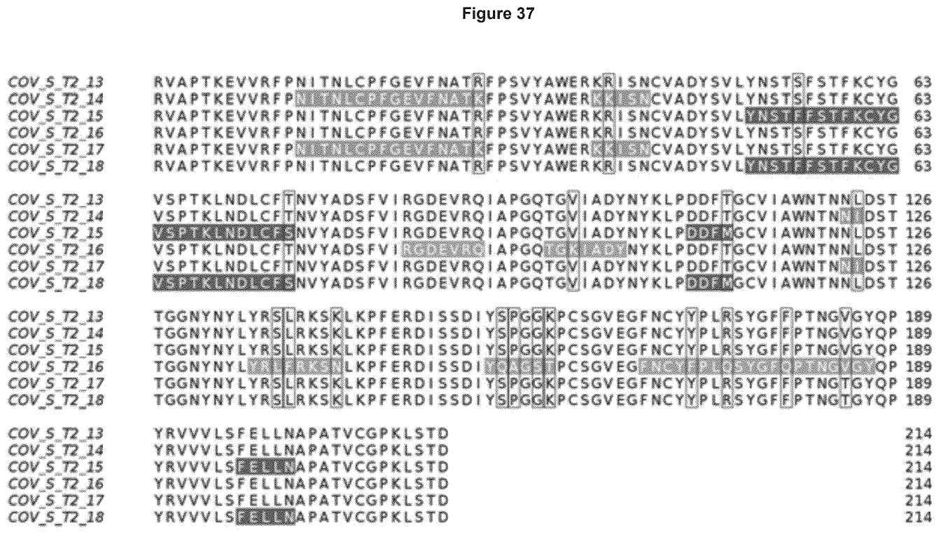

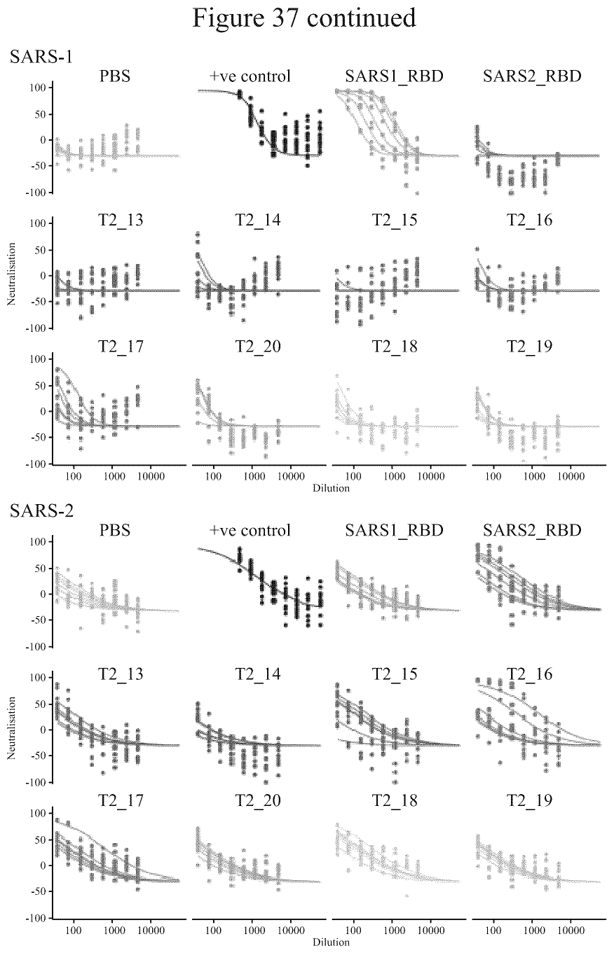

FIG. 37 Neutralisation data:
A. Sequence alignment of the vaccine designs (COV_S_T2_13-18) (SEQ ID NOS: 27-32, respectively). The epitopes are highlighted as coloured blocks. The amino acid residues differing between the designs are boxed in black.
B. Neutralisation curves of vaccine designs, SARS-1 RBD and SARS-2 RBD against SARS1 pseudotype (upper panel) and SARS2 pseudotype (lower panel). The X-axis represents the dilution of the sera and the Y-axis represent the percentage of neutralisation observed. Each curve in the plots represents an individual mouse;

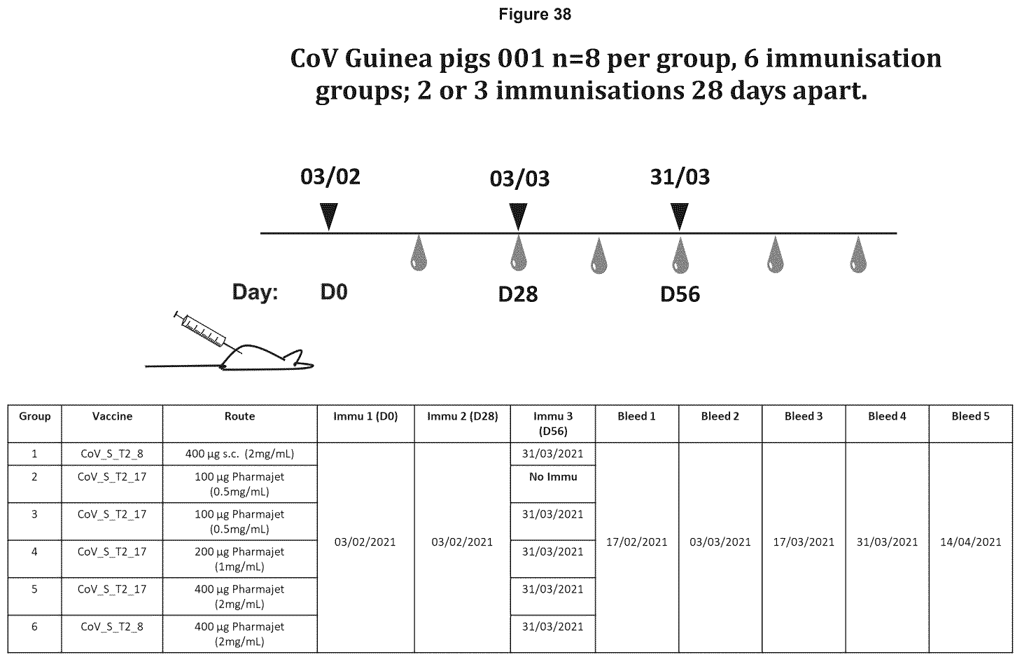

FIG. 38 represents the study protocol of a dose finding study of COV_S_T2_17 (SEQ ID NO: 31);

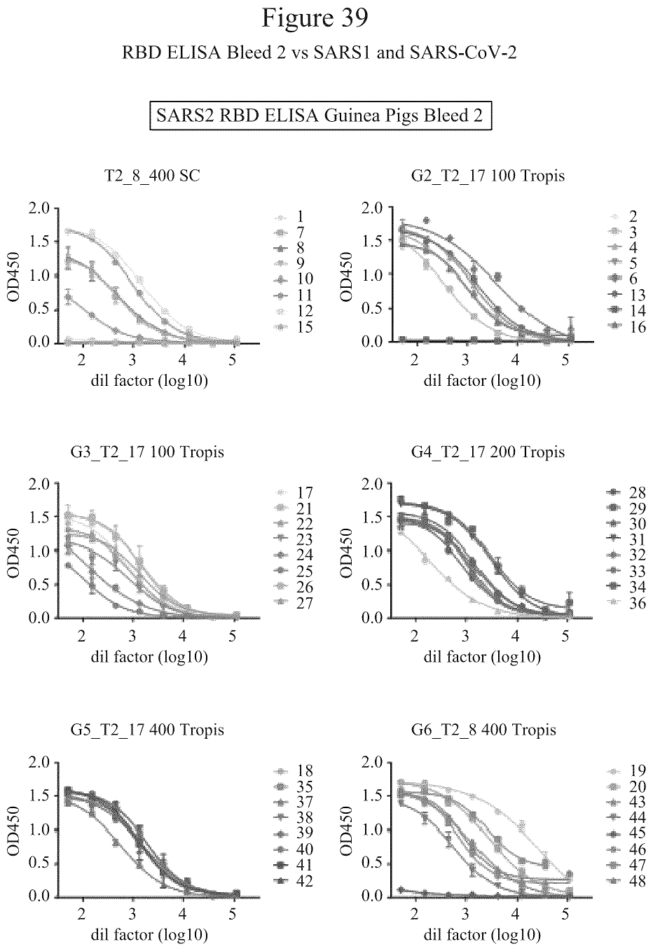

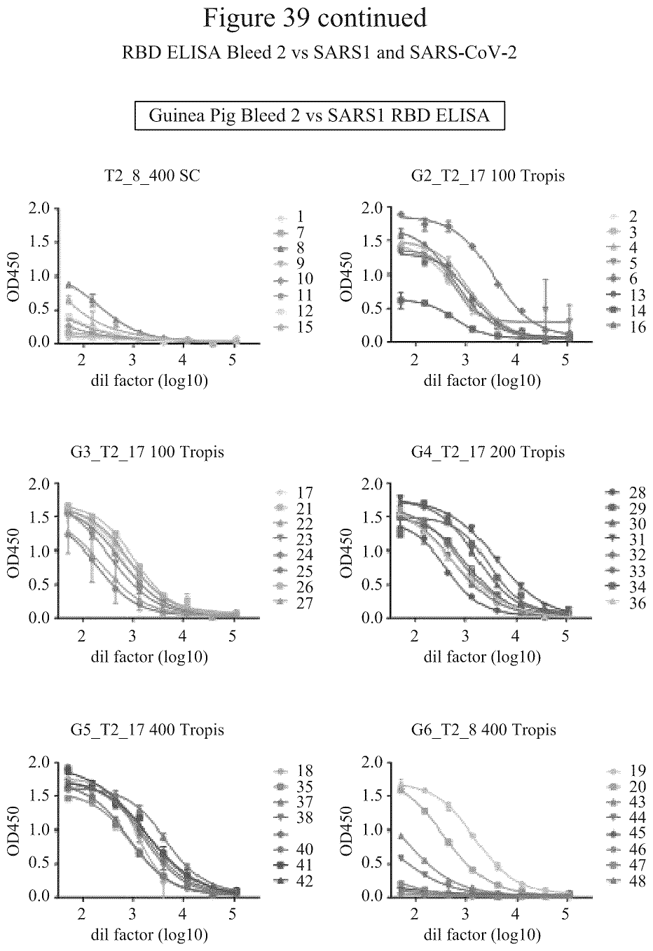

FIG. 39 shows the results of ELISA to determine the level of antibodies to the RBD of SARS-CoV-2, and SARS. Panel A (left) Plates coated with SARS-CoV-2 RBD. Panel B (right) Plates coated with SARS RBD;

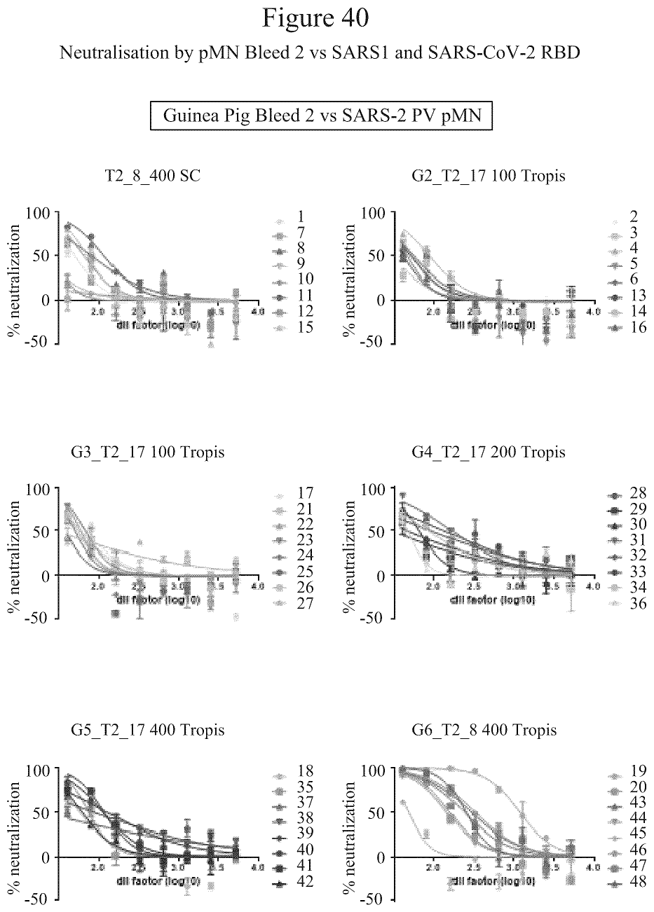

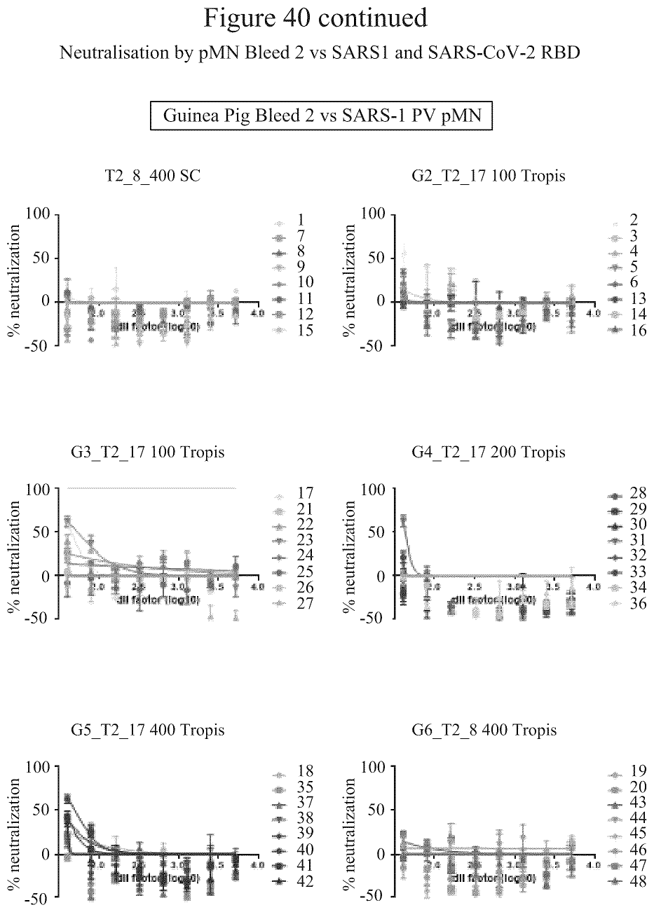

FIG. 40 shows virus neutralisation at day 28 after 1 immunisation (Pseudotype MicroNeutralisation or pMN assay). Panel A (left) Antibody neutralisation of SARS-CoV-2 28 days after 1 dose. Panel B (right) Antibody neutralisation of SARS 28 days after 1 dose;

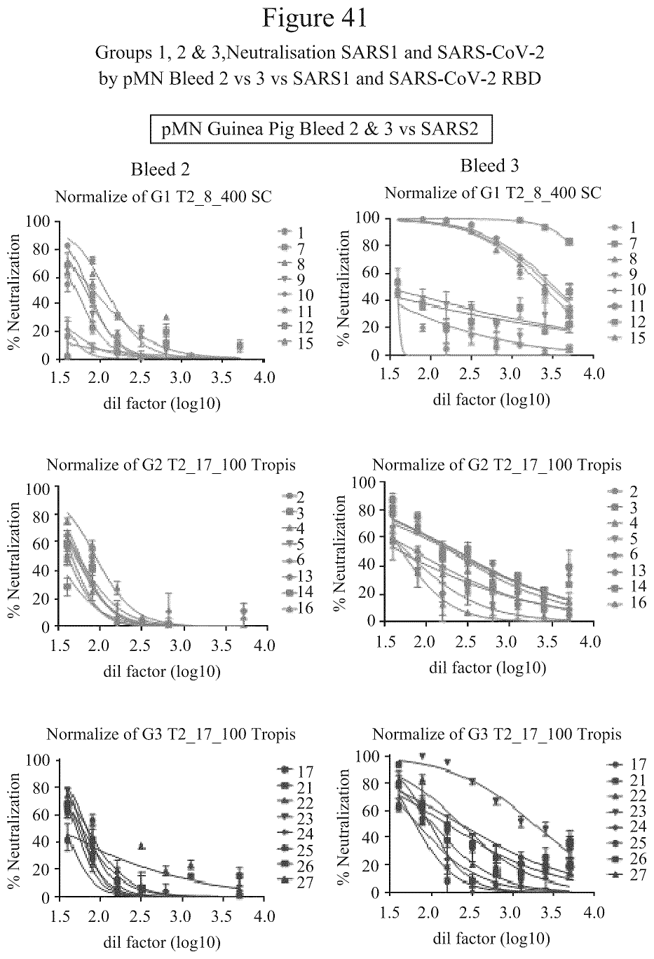

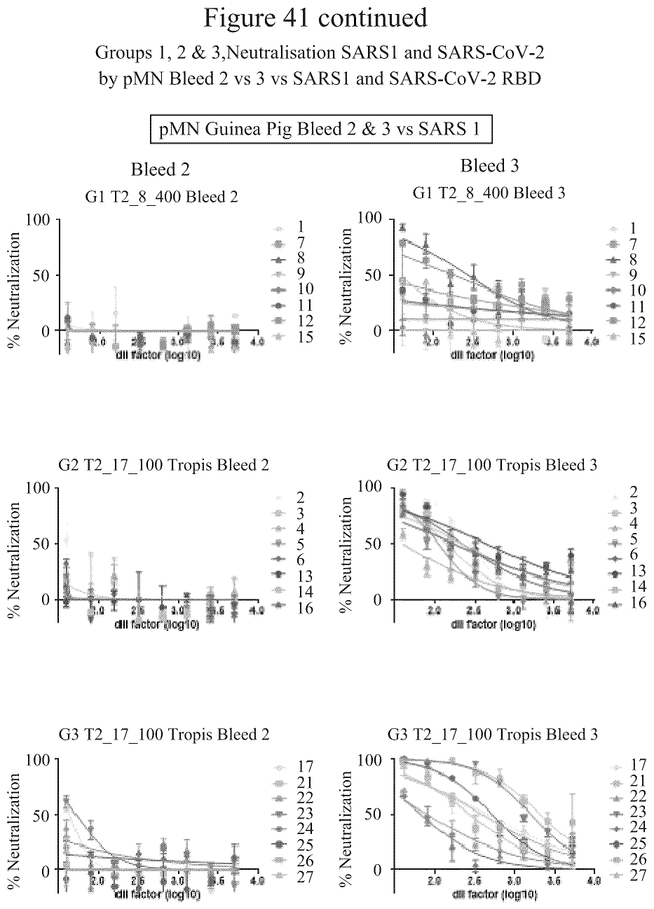

FIG. 41 shows (for Groups 1, 2, and 3) comparison of virus neutralisation responses after first to second immunisation. Panel A (left SARS-CoV-2) Comparing bleeds 2 (pre) and 3 (post) second immunisation (boost). Panel B (right SARS) Comparing bleeds 2 (pre) and 3 (post) second immunisation (boost);

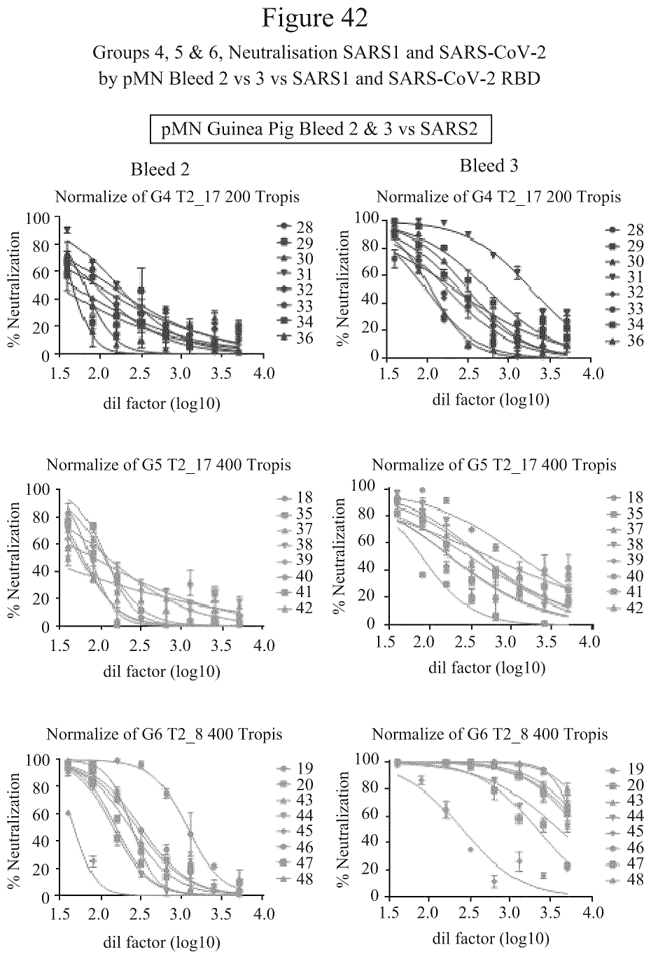

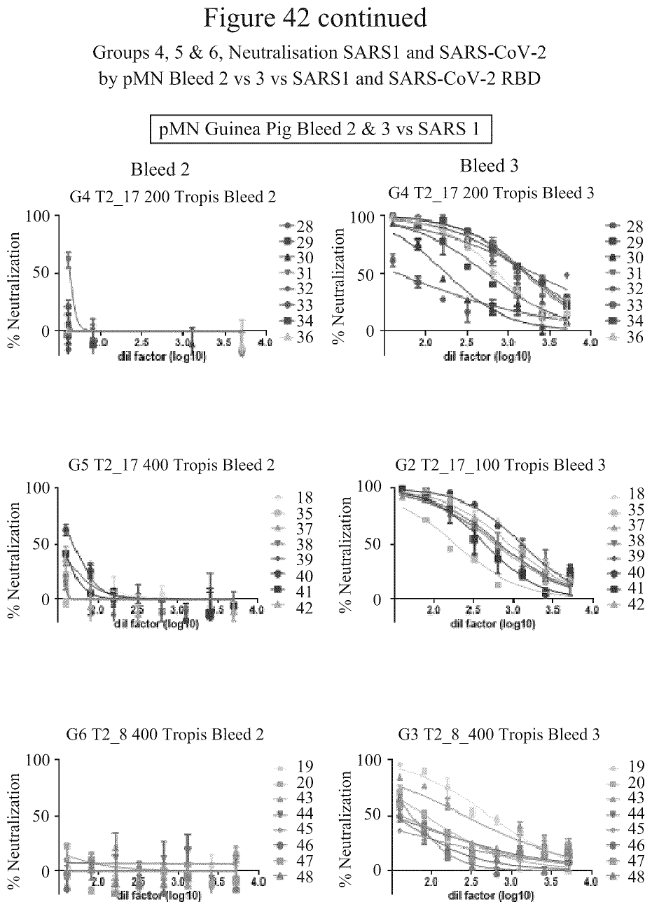

FIG. 42 shows (for groups 4, 5 and 6) comparison of virus neutralisation responses after first to second immunisation. Panel A (left SARS-CoV-2) Comparing bleeds 2 (pre) and 3 (post) second immunisation (boost). Panel B (right SARS) Comparing bleeds 2 (pre) and 3 (post) second immunisation (boost);

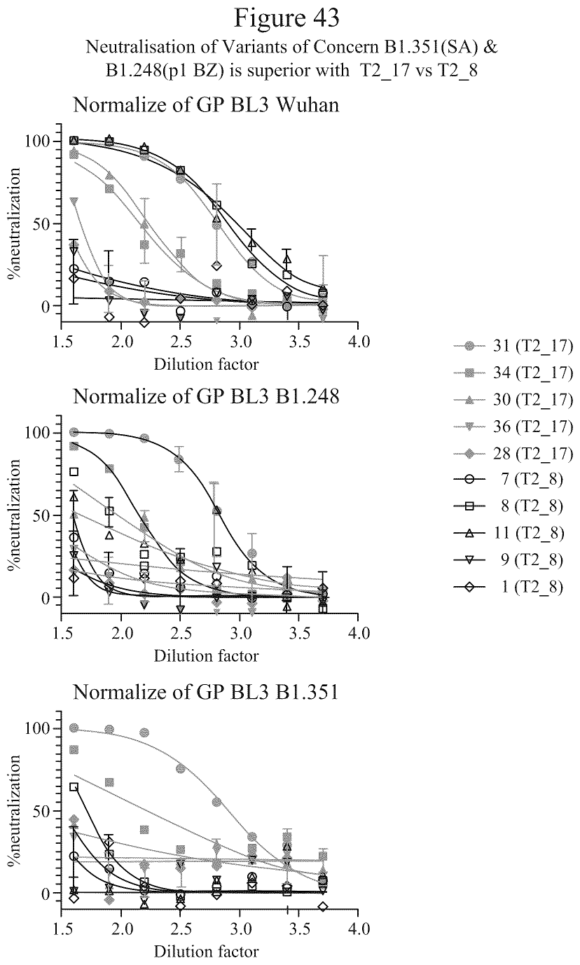

FIG. 43 shows neutralisation of variants of concern (B1.351 (SA) & B1.248 (P1 BZ) is superior with T2_17 vs T2_8);

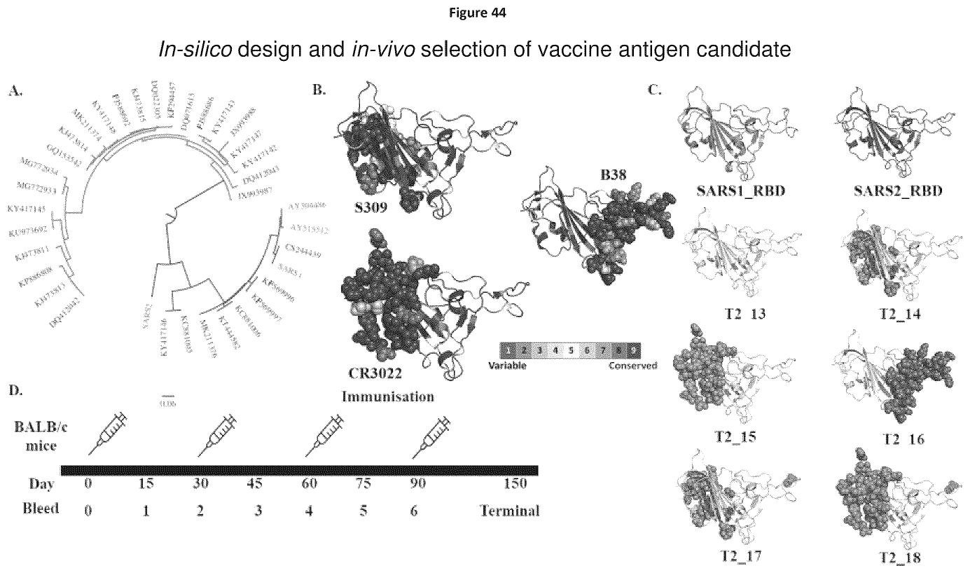

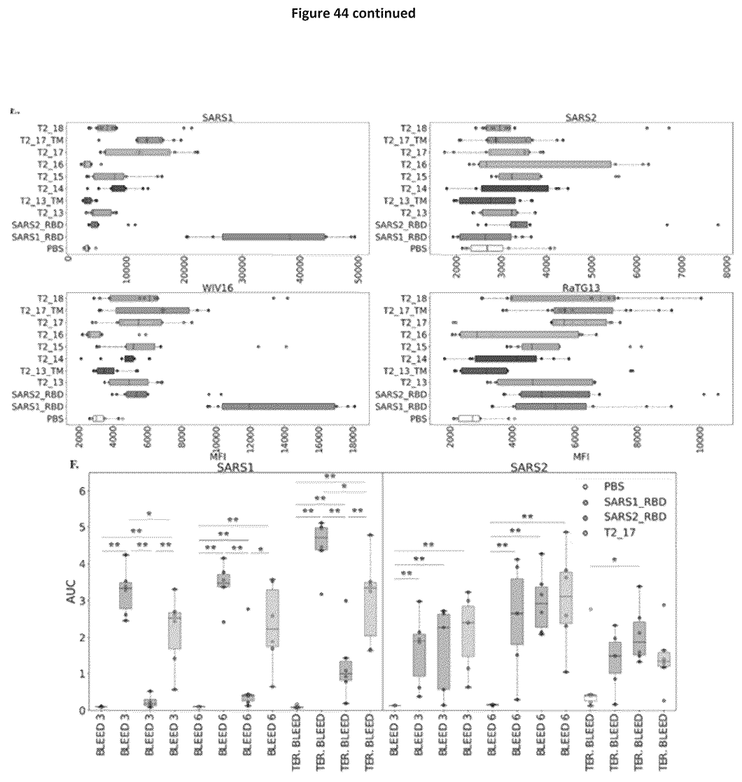

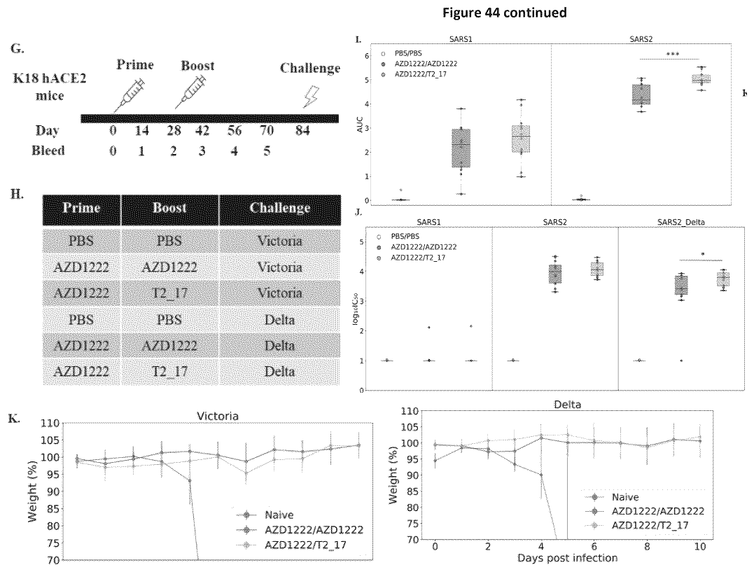

FIG. 44 shows in silico design and in-vivo selection of vaccine antigen candidate;

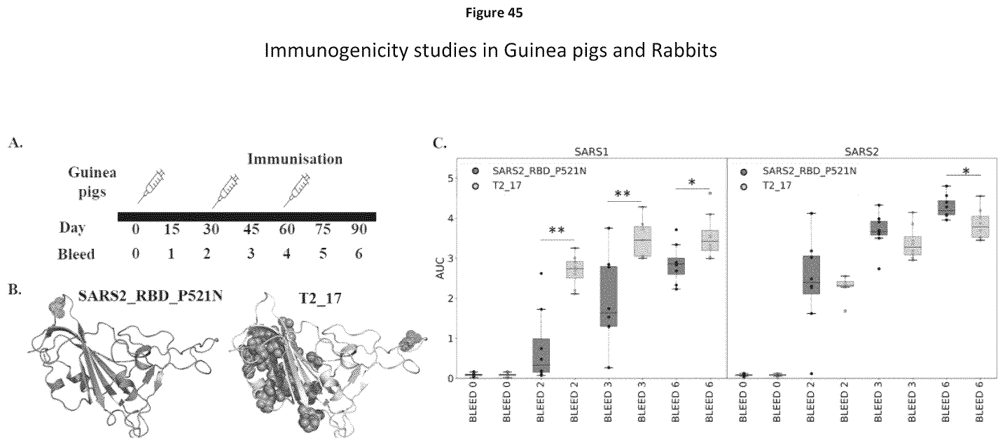

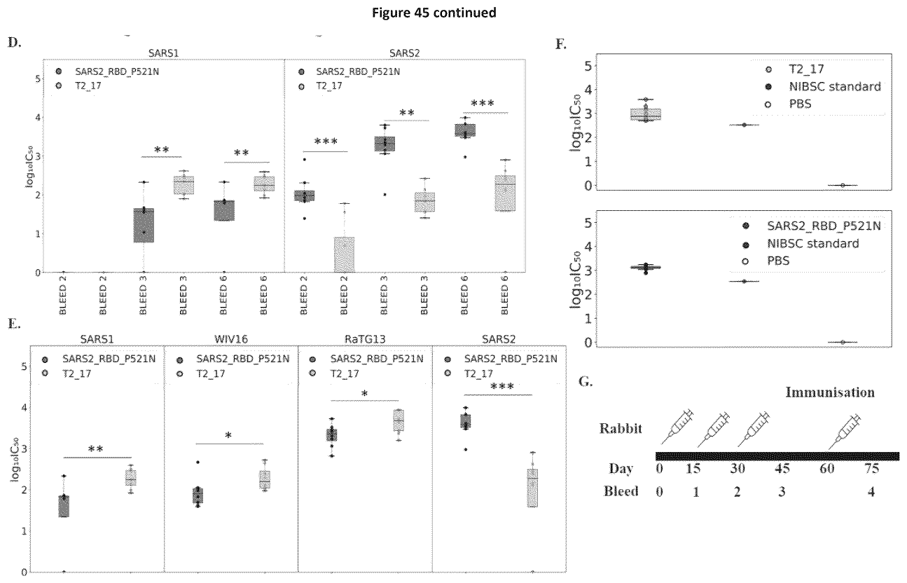

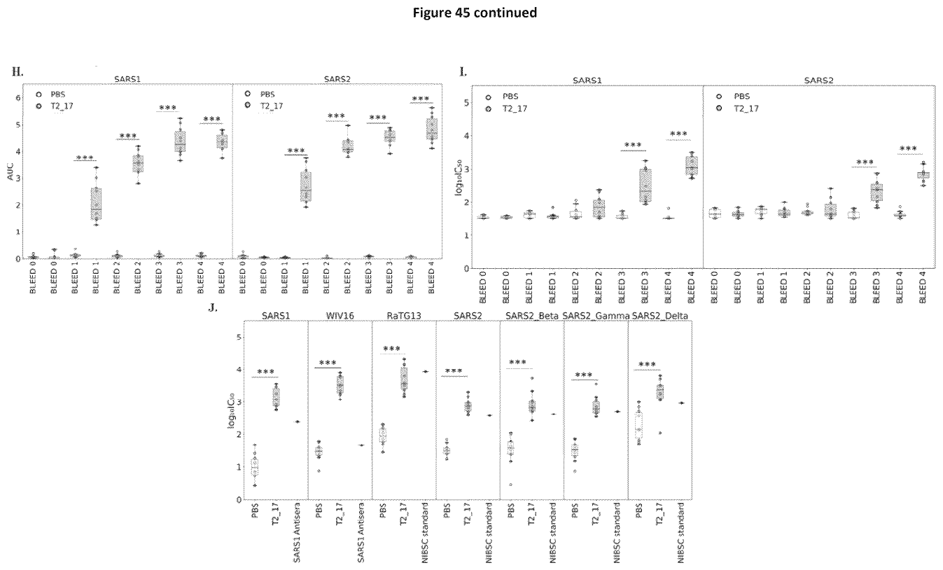

FIG. 45 shows immunogenicity studies in Guinea pigs and rabbits;

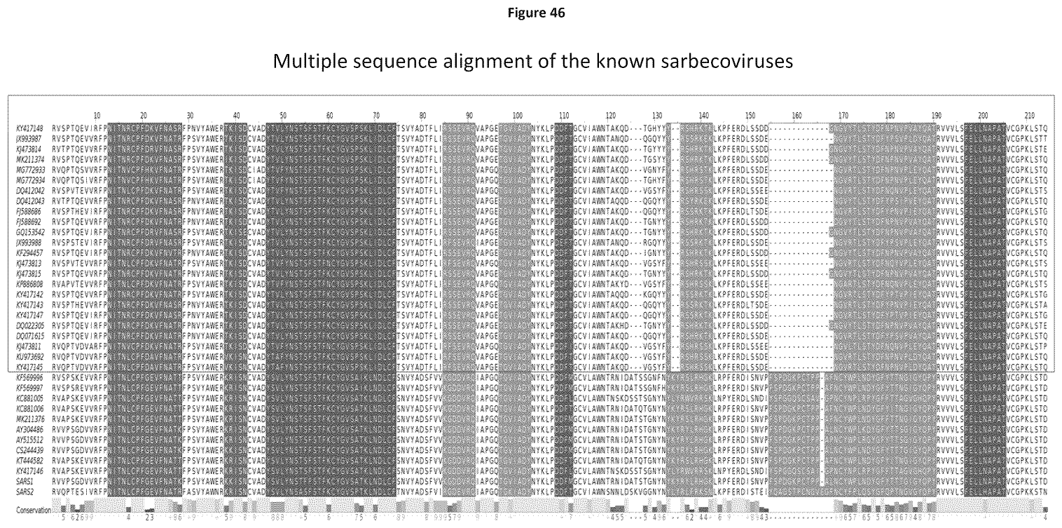

FIG. 46 shows multiple sequence alignment of the known sarbecoviruses;

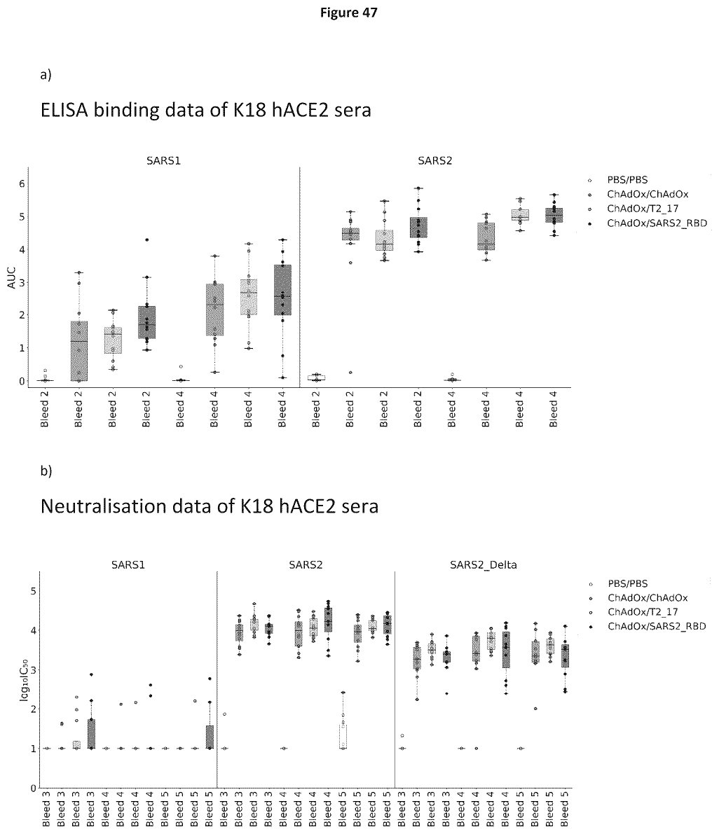

FIG. 47A shows ELISA binding data of K18 hACE2 sera;

FIG. 47B shows neutralisation data of K18 hACE2 sera; and

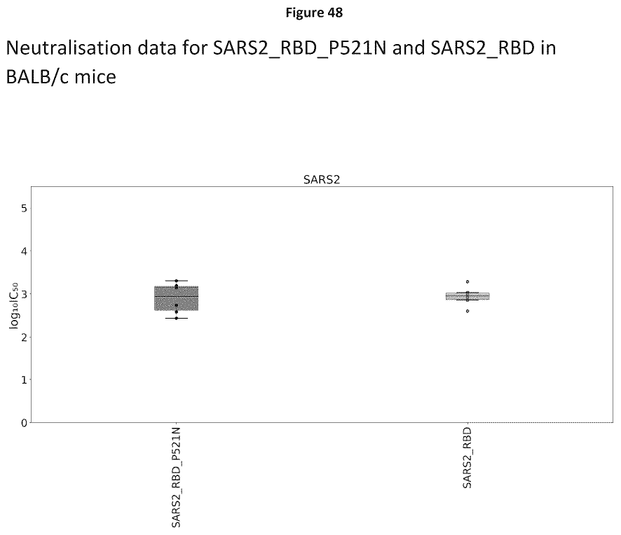

FIG. 48 shows neutralisation data for SARS2_RBD_P521N and SARS2_RBD in BALB/c mice;

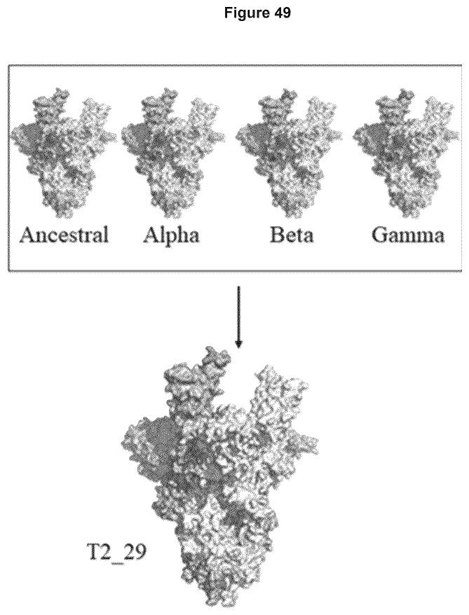

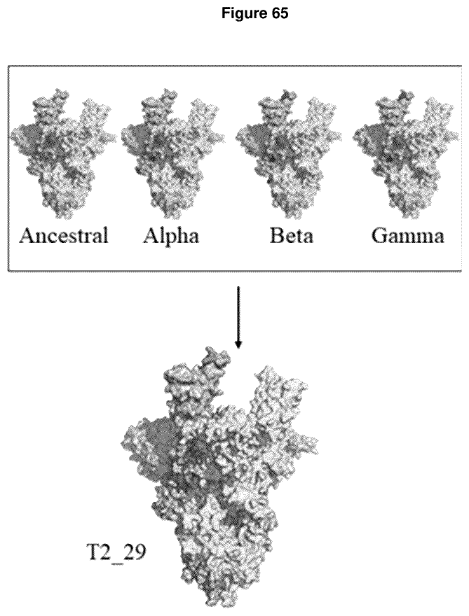

FIG. 49 shows surface representation of the extra-virion region of the spike protein of SARS-CoV-2. The three subunits are coloured in pale yellow, pale blue, and grey. The mutations reported in different variants are coloured as red. The mutations introduced in the spike vaccine antigens are coloured as orange in T2_29. The distinction between these colours can be seen in FIG. 65;

FIG. 50: Spike vaccine antigen T2_29 delivered by DNA and MVA in Guinea pigs;

FIG. 51 shows VOC RBD binding antibody levels (ELISA) of guinea pigs at bleed 4 after DNA immunisation with T2_29 constructs;

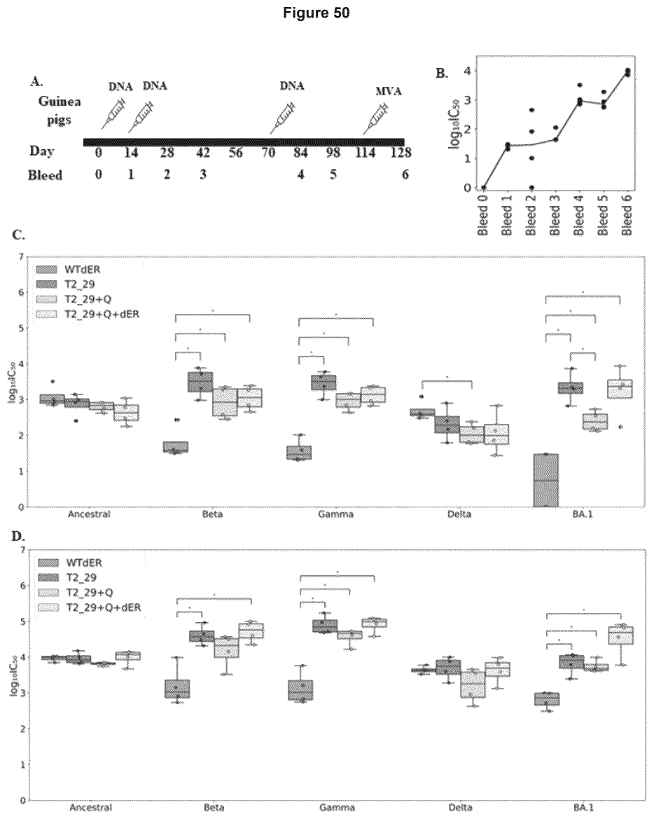

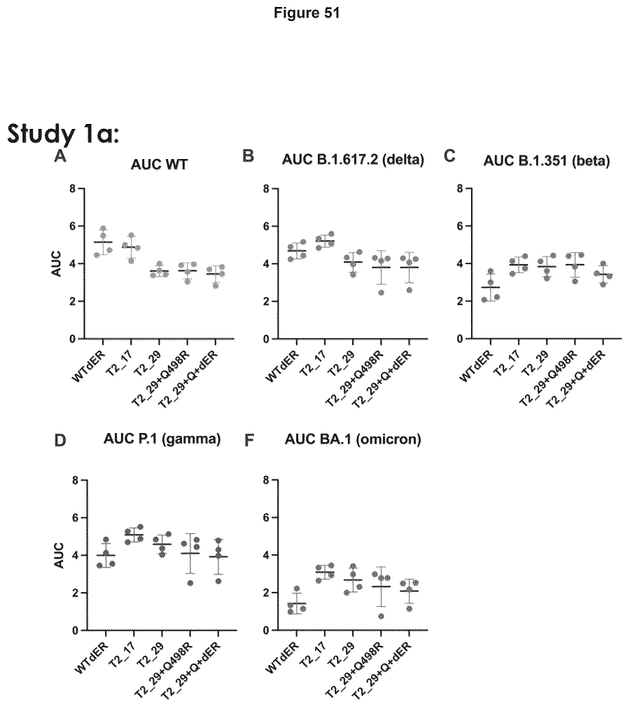

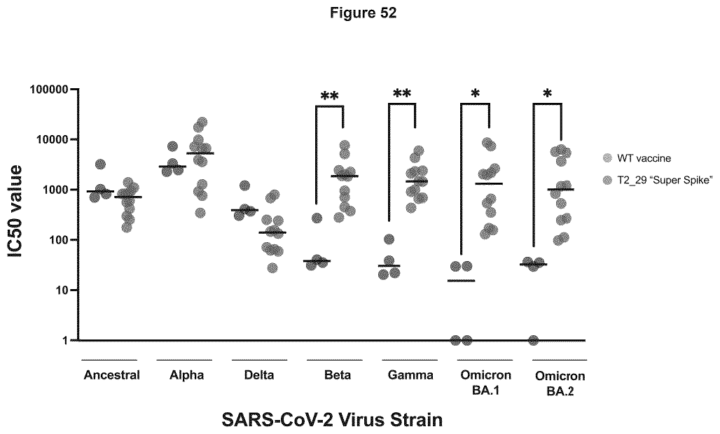

FIG. 52 shows the distribution of the neutralisation titre of guinea pig serum (at bleed 4) against Ancestral and VOCs, after DNA immunisation using WT vaccine (WTdER) and T2_29 vaccine (combined data for groups 2a, 2b, 2c);

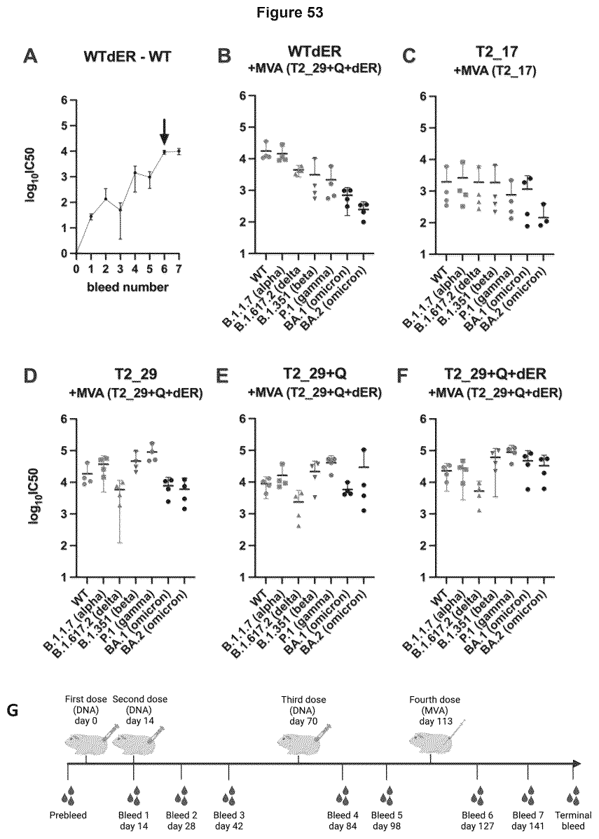

FIG. 53A shows neutralisation titre of guinea pig serum after WTdER vaccination. FIG. 53B-F shows neutralisation titre of guinea pig serum after immunisation with DNA and MVA vaccine constructs (T2-17, T2_29, and T2_29 associated). FIG. 53G shows an overview of 3×DNA and MVA boost immunisation and bleed schedule;

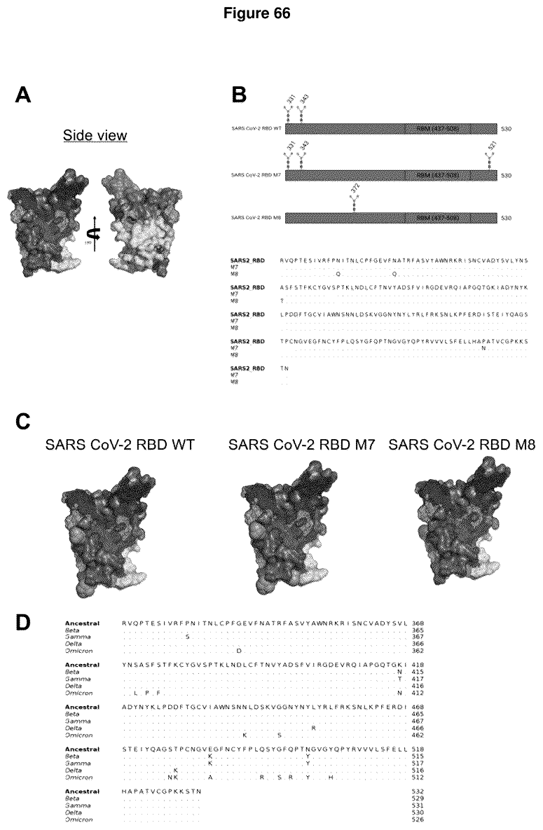

FIG. 54 shows rational immunogen design of glycan engineered SARS COV-2 RBD mutants (colour version of this figure is provided in FIG. 66);

FIG. 55 shows SARS COV-2 RBD DNA-based vaccine candidates induce humoral immune response in Balb/c mice;

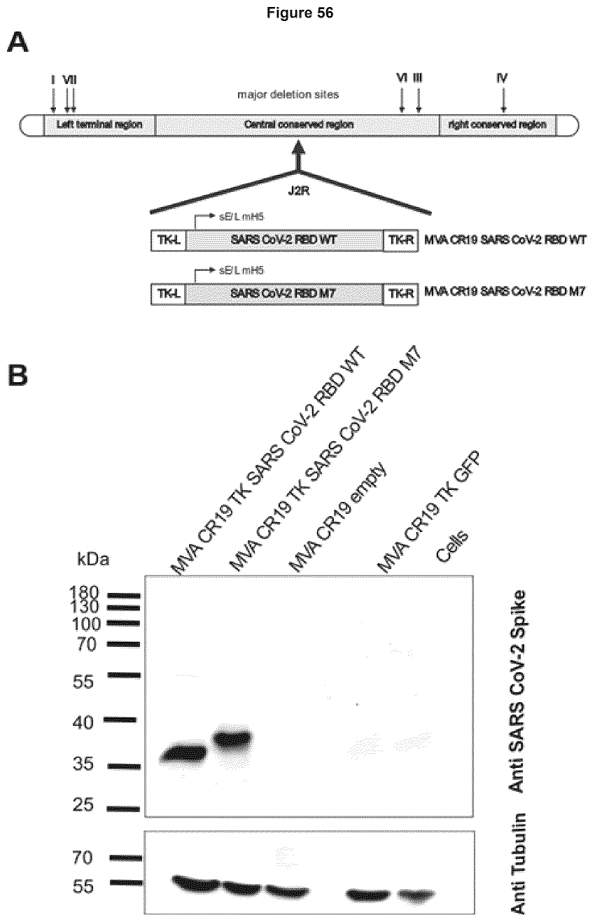

FIG. 56 shows construction and biochemical characterization of recombinant MVAs encoding for SARS COV-2 RBD WT and SARS COV-2 RBD M7 antigens;

FIG. 57 shows DNA/MVA superior to DNA/DNA regimen regarding induction of binding and neutralizing antibodies against VOCs;

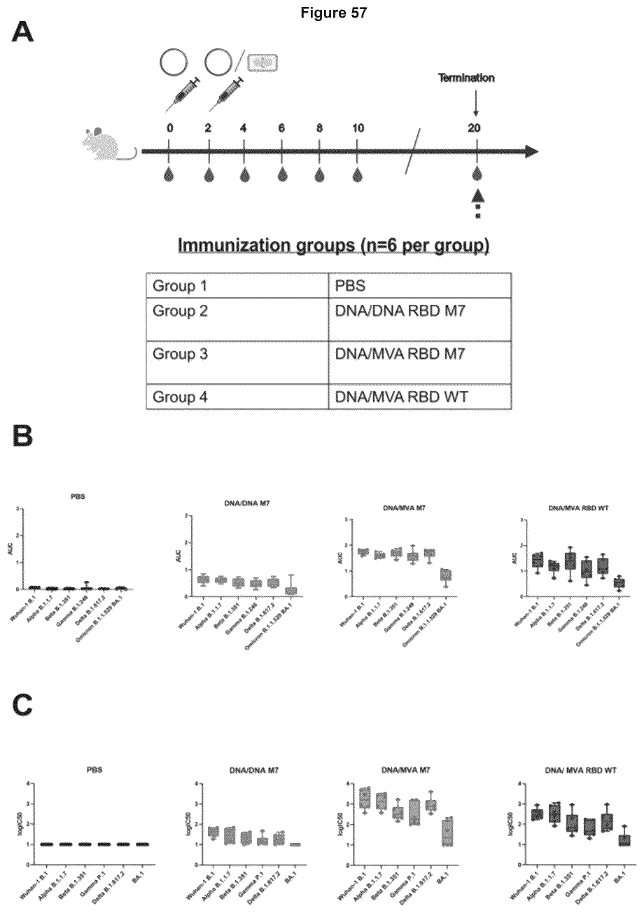

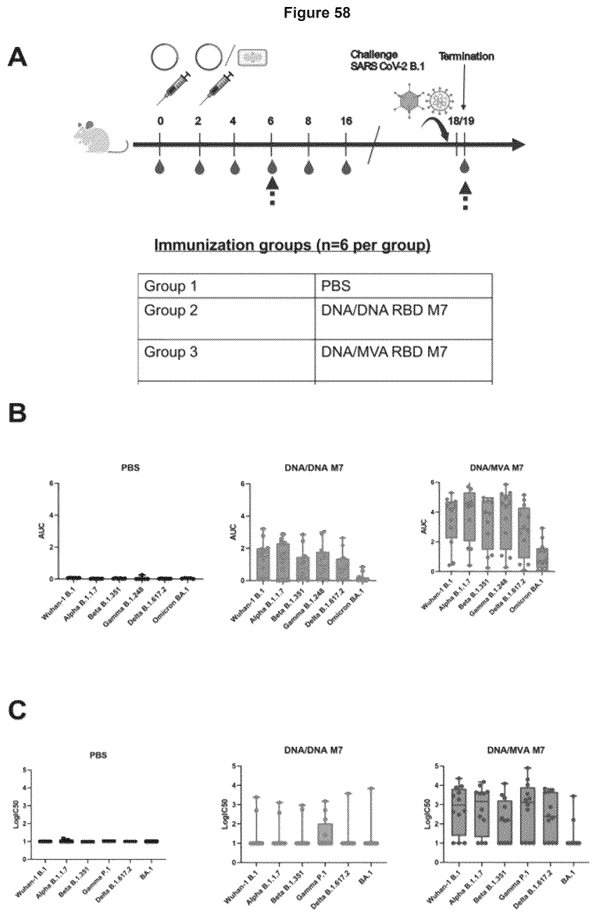

FIG. 58 shows challenge in human ACE2 transduced mice with SARS COV-2 wildtype virus;

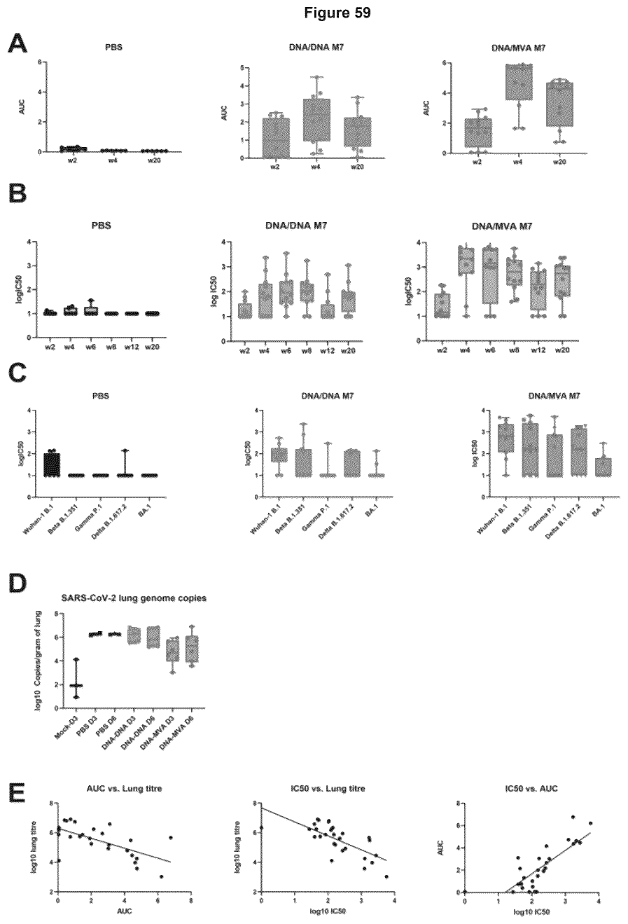

FIG. 59 shows DNA prime and MVA boost provides strong, broad and longer lasting neutralising antibody response that results in a reduction of viral load after challenge with SARS COV-2 wildtype strain;

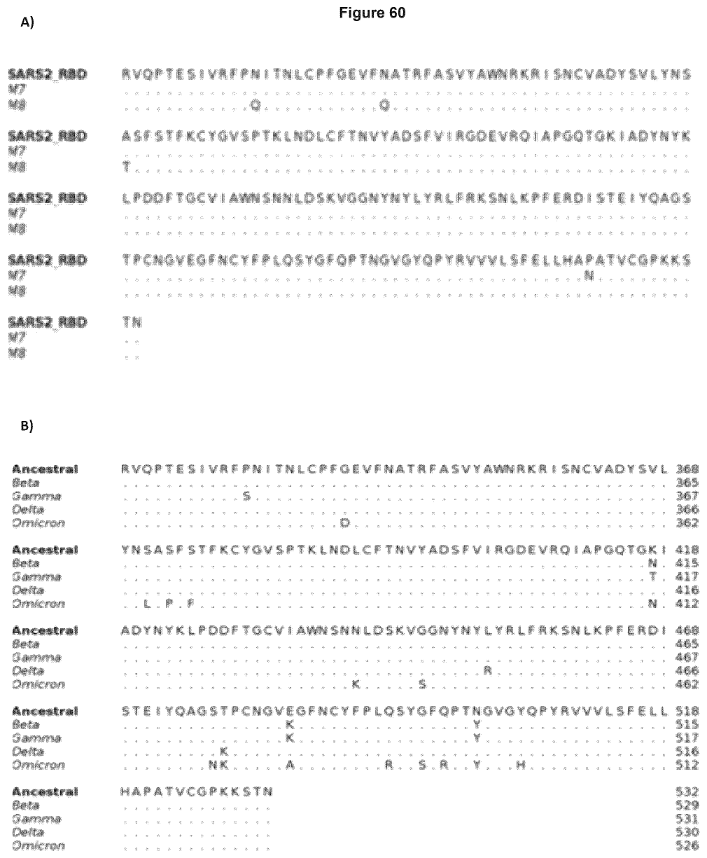

FIG. 60A shows an enlarged image of the sequence alignment of FIG. 54B, and FIG. 60B shows an enlarged image of the sequence alignment of FIG. 54D;

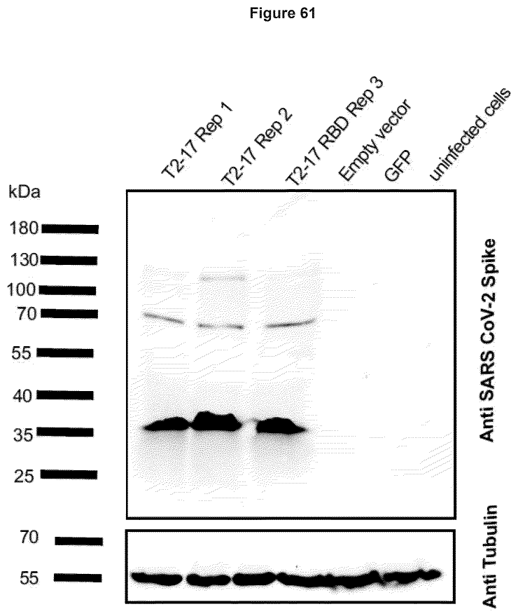

FIG. 61 shows expression analysis of HEK293T cells following transfection with pURVac T2_17 RBD;

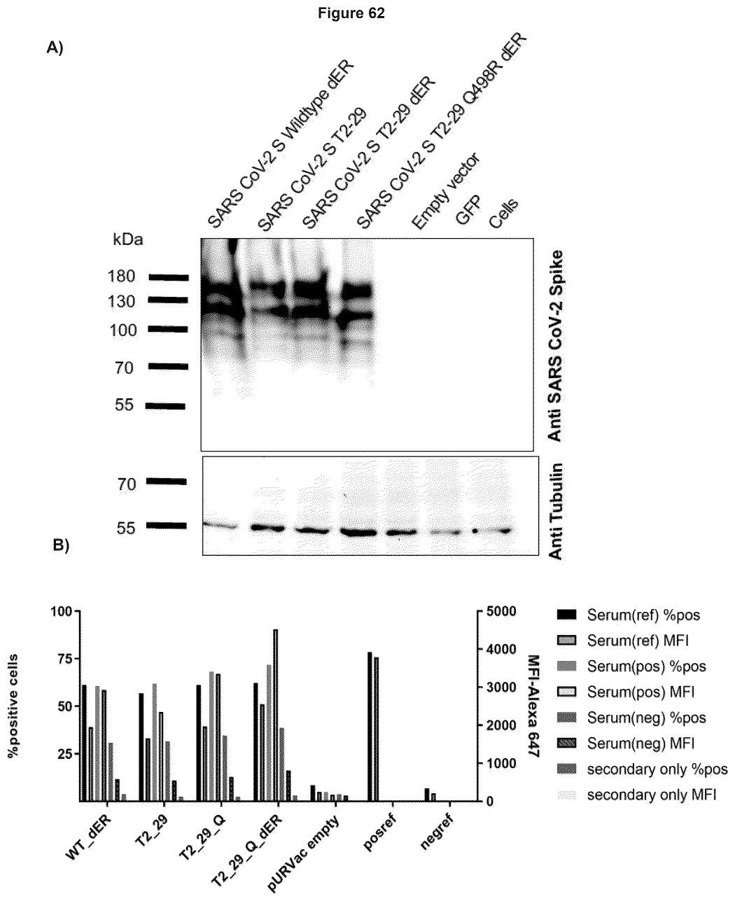

FIG. 62A shows expression analysis (Western blot) of HEK293T cells following transfection with pURVac T2_29 DNA constructs;

FIG. 62B shows expression analysis (flow cytometry) of HEK293T cells following transfection with pURVac T2_29 DNA constructs;

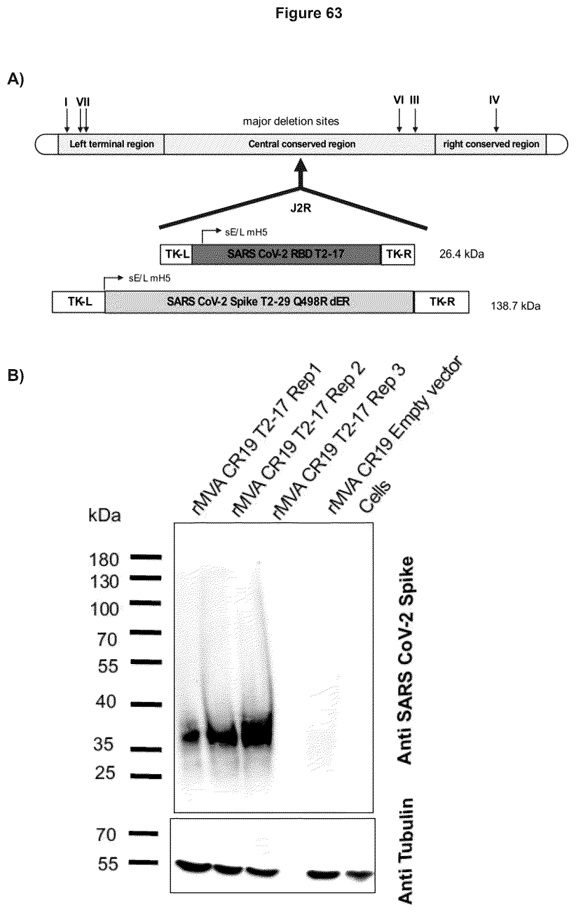

FIG. 63A shows a schematic representation of the MVA genome and design of the recombinant SARS COV-2 RBD T2_17 and SARS COV-2 Spike T2_29+Q498R+dER MVAs;

FIG. 63B shows expression analysis (Western blot) of T2_17+tPA RBD rMVA;

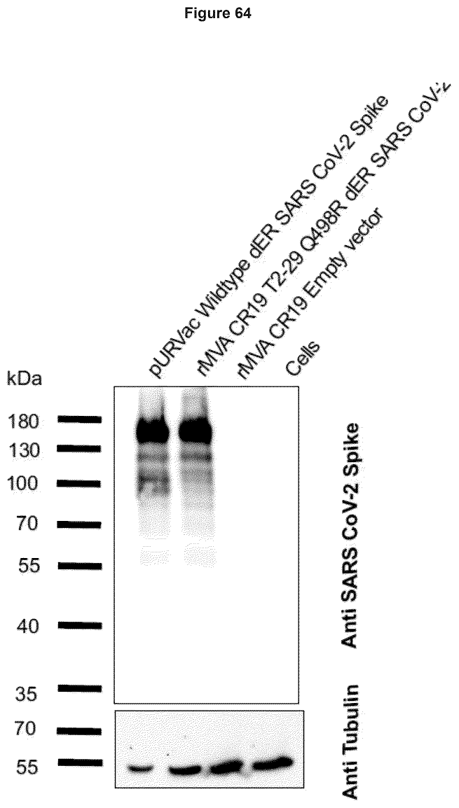

FIG. 64 shows expression analysis (Western blot) of T2_29+Q498R+dER rMVA;

FIG. 65 is a colour version of FIG. 49, and shows a surface representation of the extra-virion region of the spike protein of SARS-CoV-2;

FIG. 66 is a colour version of FIG. 54, and shows rational immunogen design of glycan engineered SARS COV-2 RBD mutants. The figure shows three epitope regions of the class 1 monoclonal antibody (mAb) B3829 (shown in red brown), class 3 mAb CR302230 (shown in yellow) and class 4 S30931 (shown in grey), which were selected for glycan engineering of the SARS COV-2 RBD ancestral sequence to generate M7 and M8 designed sequences.

TABLE of
SEQ ID NOS:

| SEQ ID NO: | Description |
|---|---|
| 1 | AY274119 (CoV_T1_1): full length S-protein |
| 2 | Nucleic acid sequence encoding amino acid sequence of SEQ ID NO: 1 |
| 3 | AY274119_tr (CoV_T2_2): truncated S-protein |
| 4 | Nucleic acid sequence encoding amino acid sequence of SEQ ID NO: 3 |
| 5 | AY274119_RBD (COV_T2_5): RBD |
| 6 | Nucleic acid sequence encoding amino acid sequence of SEQ ID NO: 5 |
| 7 | EPI_ISL_402119 (COV_T1_2): full length S-protein |
| 8 | Nucleic acid sequence encoding amino acid sequence of SEQ ID NO: 7 |
| 9 | EPI_ISL_402119_tr (CoV_T2_3): truncated S-protein |
| 10 | Nucleic acid sequence encoding amino acid sequence of SEQ ID NO: 9 |
| 11 | EPI_ISL_402119_RBD (COV_T2_6): RBD |
| 12 | Nucleic acid sequence encoding amino acid sequence of SEQ ID NO: 11 |
| 13 | Wuhan_Node1 (CoV_T2_1): full length S-protein |
| 14 | Nucleic acid sequence encoding amino acid sequence of SEQ ID NO: 13 |
| 15 | Wuhan_Node1_tr (COV_T2_4): truncated S-protein |
| 16 | Nucleic acid sequence encoding amino acid sequence of SEQ ID NO: 15 |
| 17 | Wuhan_Node1_RBD (CoV_T2_7): RBD |
| 18 | Nucleic acid sequence encoding amino acid sequence of SEQ ID NO: 17 |
| 19 | Sequence of pEVAC Multiple Cloning Site (MCS) |
| 20 | Entire Sequence of pEVAC |
| 21 | Amino acid sequence of the SARS envelope protein |
| 22 | COV_E_T2_1 (a designed Sarbecovirus sequence) |
| 23 | COV_E_T2_2 (a designed SARS2 sequence) |
| 24 | COV_M_T2_1/1-221 Sarbeco_M_root-Sarbecovirus root ancestor |
| 25 | COV_M_T2_2/1-222 Sarbeco_M_Node88b_epitope_optimised |
| 26 | COV_M_T1_1/1-222 NC_045512.2 SARS2 reference sequence |
| 27 | COV_S_T2_13 (designed S protein RBD sequence) |
| 28 | COV_S_T2_14 (designed S protein RBD sequence) |
| 29 | COV_S_T2_15 (designed S protein RBD sequence) |
| 30 | COV_S_T2_16 (designed S protein RBD sequence) |
| 31 | COV_S_T2_17 (designed S protein RBD sequence) |
| 32 | COV_S_T2_18 (designed S protein RBD sequence) |
| 33 | Designed S protein RBD sequence M7 |
| 34 | Designed S protein RBD sequence M8 |
| 35 | Designed S protein RBD sequence M9 |
| 36 | Designed S protein RBD sequence M10 |
| 37 | Nucleic acid sequence encoding designed S protein RBD sequence M7 |
| 38 | Nucleic acid sequence encoding designed S protein RBD sequence M8 |
| 39 | Nucleic acid sequence encoding designed S protein RBD sequence M9 |
| 40 | Nucleic acid sequence encoding designed S protein RBD sequence M10 |
| 41 | SARS2 reference E protein sequence |
| 42 | COV_E_T2_3 (SARS2_mutant) |
| 43 | COV_E_T2_4 (Env1_mutant) |
| 44 | COV_E_T2_5 (Env2_mutant) |
| 45 | YP_009724397.2/1-419 nucleocapsid phosphoprotein [SARS-CoV-2] (reference |
| | sequence) |
| 46 | COV_N_T2_1/1-418 Node1b 321-323 deleted |
| 47 | COV_N_T2_2/1-417 epitope optimised 321-323 deleted |
| 48 | COV_M_T2_3 |
| 49 | COV_M_T2_4 |
| 50 | COV_M_T2_5 |
| 51 | Amino acid sequence of “Ralf RBD protein” (Leader-RBD-Tag) |
| 52 | Amino acid sequence of full length S protein for strain |
| | EPI_ISL_402130_Wuhan |
| 53 | Amino acid sequence for designed full length S protein COV_S_T2_29 (“VOC |
| | Chimera” or “Super_spike”) |
| 54 | Amino acid sequence for designed full length S protein COV_S_T2_29, but |
| | with cysteine residues at positions 410 and 984 (i.e. G410C and P984C), |
| | which correspond to positions 413 and 987, respectively, of SEQ ID NO: 52 |
| 55 | COV_S_T2_19 (designed S protein RBD sequence) |
| 56 | COV_S_T2_20 (designed S protein RBD sequence) |
| 57 | residues (i) of a discontinuous epitope present in COV_S_T2_14 and |
| | COV_S_T2_17: NITNLCPFGEVFNATK; |
| 58 | residues (ii) of a discontinuous epitope present in COV_S_T2_14 and |
| | COV_S_T2_17: KKISN; |
| 59 | residues (iii) of a discontinuous epitope present in COV_S_T2_14 and |
| | COV_S_T2_17: NI; |
| 60 | residues (i) of a discontinuous epitope present in COV_S_T2_15 and |
| | COV_S_T2_18: YNSTFFSTFKCYGVSPTKLNDLCFS; |
| 61 | residues (ii) of a discontinuous epitope present in COV_S_T2_15 and |
| | COV_S_T2_18: DDFM; |
| 62 | residues (iii) of a discontinuous epitope present in COV_S_T2_15 and |
| | COV_S_T2_18: FELLN; |
| 63 | residues (i) of a discontinuous epitope present in COV_S_T2_16: RGDEVRQ; |
| 64 | residues (ii) of a discontinuous epitope present in COV_S_T2_16: TGKIADY; |
| 65 | residues (iii) of a discontinuous epitope present in COV_S_T2_16: |
| | YRLFRKSN; |
| 66 | residues (iv) of a discontinuous epitope present in COV_S_T2_16: YQAGST; |
| 67 | residues (v) of a discontinuous epitope present in COV_S_T2_16: |
| | FNCYFPLQSYGFQPTNGVGY. |
| 68 | residues (i) of a discontinuous epitope present in COV_S_T2_13: |
| | NITNLCPFGEVFNATR |
| 69 | residues (ii) of a discontinuous epitope present in COV_S_T2_13: KRISN |
| 70 | residues (iii) of a discontinuous epitope present in COV_S_T2_13: NL |
| 71 | residues (i) of a discontinuous epitope present in COV_S_T2_13: |
| | YNSTSFSTFKCYGVSPTKLNDLCFT |
| 72 | residues (ii) of a discontinuous epitope present in COV_S_T2_13: DDFT |
| 73 | residues (ii) of a discontinuous epitope present in COV_S_T2_13: TGVIADY |
| 74 | residues (iii) of a discontinuous epitope present in COV_S_T2_13: |
| | YRSLRKSK |
| 75 | residues (iv) of a discontinuous epitope present in COV_S_T2_13: YSPGGK |
| 76 | residues (v) of a discontinuous epitope present in COV_S_T2_13: |
| | FNCYYPLRSYGFFPTNGVGY |
| 77 | residues (v) of a discontinuous epitope present in COV_S_T2_17, 18: |
| | FNCYYPLRSYGFFPTNGTGY |
| 78 | Nucleic acid encoding COV_S_T2_13 |
| 79 | Nucleic acid encoding COV_S_T2_14 |
| 80 | Nucleic acid encoding COV_S_T2_15 |
| 81 | Nucleic acid encoding COV_S_T2_16 |
| 82 | Nucleic acid encoding COV_S_T2_17 |
| 83 | Nucleic acid encoding COV_S_T2_18 |
| 84 | Nucleic acid encoding COV_S_T2_19 |
| 85 | Nucleic acid encoding COV_S_T2_20 |
| 86 | T2_17 + pEVAC Expression Vector |
| 87 | Amino acid sequence for designed full length S protein COV_S_T2_29, but |
| | with arginine residue at position 498 (i.e. Q498R) of SEQ ID NO:52, which |
| | corresponds to position 495, of SEQ ID NO: 53 (COV_S_T2_29) |
| 88 | Amino acid sequence for designed full length S protein |
| | COV_S_T2_29 + Q498R with 19 amino acid C-terminal truncation (dER) |
| 89 | COV_S_T2_29 nucleic acid sequence |
| 90 | COV_S_T2_29 + Q498R nucleic acid sequence |
| 91 | COV_S_T2_29 + Q498R + dER nucleic acid sequence |
| 92 | CoV_S_T2_17 + tPA signal sequence (amino acid sequence) |
| 93 | CoV_S_T2_17 + tPA signal sequence (nucleic acid sequence) |
| 94 | pURVAC_T2_17 + tPA (nucleic acid sequence) |
| 95 | pURVAC_COV_S_T2_29 + Q498R + dER (nucleic acid sequence) |
| 96 | MVA transfer vector |
| 97 | pMVA Trans TK mH5 T2_17 + tPA |
| 98 | pMVA Trans TK mH5 T2_29 + Q498R + dER |
| 99 | SARS1 S protein |
| 100 | SARS2 RaTG13 S protein |
| 101 | SARS2 EPI_ISL_402119 S protein |
| 102 | SARS2 EPI_ISL_402132 S protein |
| 103 | SARS2 EPI_ISL_403936 S protein |
| 104 | SARS2 EPI_ISL_404253 S protein |
| 105 | Consensus sequence S protein |
| 106 | NL63_Alpha E protein |
| 107 | 229E_Alpha E protein |
| 108 | HKU1_Beta E protein |
| 109 | HKU1_Beta E protein |
| 110 | KF600630_MERS_Beta E protein |
| 111 | Consensus E protein |
| 112 | Consensus E protein |
| 113 | Consensus E protein |
| 114 | SARS2 M protein |
| 115 | WIV16 S protein RBD |
| 116 | RaTG13 S protein RBD |
| 117 | SARS2 N1 protein |
| 118 | SARS1 N protein |
| 119 | EPI_ISL_402119_RBD (COV_T2_6) with additional C-terminal Lysine |
| | residue |

We have developed vaccines that protect against Coronaviruses, such as SARS-CoV-2 and SARS-CoV-1, which have the potential to cause future outbreaks from zoonotic reservoirs. We have designed antigens to induce immune responses against the Sarbecoviruses (i.e. β-Coronavirus, Lineage B) in order to protect against the current pandemic and future outbreaks of related Coronaviruses.

A major concern for coronavirus vaccines is disease enhancement (Tseng et al. (2012) “Immunization with SARS Coronavirus Vaccines Leads to Pulmonary Immunopathology on Challenge with the SARS Virus”. PLOS ONE 7 (4): e35421). We have modified our antigens to avoid antibody dependant enhancement (ADE) (or ADE-like pro-inflammatory responses) and hyper-activation of the complement pathway.

DNA sequences encoding the antigens are optimised for expression in mammalian cells before inserting into a DNA plasmid expression vector, such as pEVAC. The pEVAC vector is a flexible vaccine platform and any combination of antigens can be inserted to produce a different vaccine. A previous version was used in a SARS-1 clinical trial (Martin et al, Vaccine 2008 25:633). This platform is clinically proven and GMP compliant allowing rapid scale-up. The DNA vaccine may be administered using pain-free needleless technology causing patients' cells to produce the antigens, which are recognised by the immune system to induce durable protection against SARS-CoV-2 and future outbreaks of related Coronaviruses.

While high affinity monoclonal antibodies are capable of protecting animals from SARS virus infection (Traggiai, et al. “An efficient method to make human monoclonal antibodies from memory B cells: potent neutralization of SARS coronavirus”. Nat Med 10, 871-875 (2004)), a robust antibody response in early infection in humans is associated with COVID-19 disease progression (Zhao et al, medRxiv: https://doi.org/10.1101/2020.03.02.20030189). Importantly, after recovery from infection and re-challenge of primates with SARS, lung pathology became more severe on secondary exposure, despite limited replication of the virus (Clay et al, “Primary Severe Acute Respiratory Syndrome Coronavirus Infection Limits Replication but Not Lung Inflammation upon Homologous Rechallenge”, J Virol. 2012 April; 86(8): 4234-4244). There is a growing body of evidence of adverse effects of vaccine induced Antibody Dependant Enhancement (ADE) due to post-vaccination infection (Peeples, Avoiding pitfalls in the pursuit of a COVID-19 vaccine, PNAS Apr. 14, 2020 117 (15) 8218-8221). Non-neutralizing antibodies to S-protein may enable an alternative infection pathway via Fc receptor-mediated uptake (Wan et al. Journal of Virology. 2020, 94(5): 1-13). These and other reports underline the importance of discriminating between viral antigen structures that induce protective anti-viral effects and those which trigger pro-inflammatory responses. Thus, careful selection and modification of vaccine antigens and the type of vaccine vector that induce protective anti-viral effects, without enhancing lung pathology, is paramount.

Vaccine sequences described herein offer safety from ADE (or ADE-like pro-inflammatory responses), and also increase the breadth of the immune response that can be extended to SARS-CoV-2, SARS and related Bat Sarbecovirus Coronaviruses, which represent future pandemic threats.

Antigens encoded by vaccine sequences described herein have precision immunogenicity, are devoid of ADE sites, and are versatile and compatible with a great number of vaccine vector technologies. DNA molecules may be delivered by PharmaJet's needleless-delivery device with demonstrated immunogenicity in advanced clinical trials for other viruses and cancer, or by other DNA delivery such as electroporation or direct injection. Alternatively, the vaccine inserts can be conveniently swapped out to other viral vector, or RNA delivery platforms, which may be easily scaled for greater capacity production or to induce immune responses with different characteristics.

We have designed Coronavirus antigens to induce a highly specific immune response that not only avoids deleterious immune responses induced by the virus, but will provide broader protection, for SARS-CoV-2, SARS-1 and other zoonotic Sarbeco-Coronaviruses. By using libraries of multiple antigens, we are able to down-select the optimal antigenic structures of each class (for instance RBD, E, and M proteins) and to combine the best in class to maximise the breadth of protection from Coronaviruses, by recruiting B- and T-cell responses against multiple targets.

### EXAMPLE 1—VACCINE SEQUENCES

The CoV S-protein is a trimeric transmembrane glycoprotein essential for the entry of the virus particles into the host cell. The S-protein comprises two domains, the S1 domain responsible for ACE-2 receptor binding, and the S2 domain, responsible for fusion of the viral and cell membranes. The S-protein is the main target for immunisation. However, evidence has shown antibody dependent enhancement (ADE) of SARS-CoV infections, in particular of the S-protein, resulting in enhanced infection and immune evasion, and/or resulting proinflammatory responses. The S-protein contains non-neutralising epitopes which are bound by antibodies. This immune diversion results in enhanced disease progression due to the inability of the immune system to neutralise the pathogen. ADE can also increase infectivity of the pathogen into host cells. Neutralising antibodies produced after an initial infection of SARS-CoV may be non-neutralising to a second infection with a different SARS-CoV strain.

The high genetic similarity between SARS-CoV and SARS-CoV-2 means that it is possible to map boundaries of the S1 and S2 domains, as well as the RBD, onto a novel design scaffold. The applicant has generated a novel sequence for an S-protein, called CoV_T2_1 (also referred to as Wuhan-Node-1), which includes modifications to improve its immunogenicity, and to remove or mask epitopes that are responsible for ADE (or ADE-like pro-inflammatory responses).

This example provides amino acid and nucleic acid sequences of full length S-protein, truncated S-protein (tr, missing the C-terminal part of the S2 sequence), and the receptor binding domain (RBD) for:
SARS-TOR2 isolate AY274119;
SARS_COV_2 isolate-hCov-19/Wuhan/LVDC-HB-01/2019 (EPI_ISL_402119); and
embodiments of the invention, termed “CoV_T2_1” (or “Wuhan_Node1”).

The CoV_T2_1 (Wuhan_Node1) sequences include modifications to provide effective vaccines that induce a broadly neutralising immune response to protect against diseases caused by CoVs, especially β-CoVs, such as SARS-CoV and SARS-CoV-2. The vaccines also lack non-neutralising epitopes that may result in virus immune evasion and disease progression by ADE (or ADE-like pro-inflammatory responses).

The following amino acid and nucleic acid sequences are provided in this example:

SARS-TOR2 isolate AY274119:
AY274119 (CoV_T1_1):
   full length S-protein (SEQ ID NO:1) and nucleic acid encoding full length S-protein (SEQ ID NO:2)
AY274119_tr (CoV_T2_2):
   truncated S-protein (SEQ ID NO:3) and nucleic acid encoding truncated S-protein (SEQ ID NO:4)
AY274119_RBD (COV_T2_5):
   RBD (SEQ ID NO:5) and nucleic acid encoding RBD (SEQ ID NO:6)
      SARS_COV_2 isolate-hCov-19/Wuhan/LVDC-HB-01/2019 (EPI_ISL_402119):
EPI_ISL_402119 (CoV_T1_2):
   full length S-protein (SEQ ID NO:7) and nucleic acid encoding full length S-protein (SEQ ID NO:8)
EPI_ISL_402119_tr (CoV_T2_3):
   truncated S-protein (SEQ ID NO:9) and nucleic acid encoding truncated S-protein (SEQ ID NO:10)
EPI_ISL_402119_RBD (COV_T2_6):
   RBD (SEQ ID NO:11) and nucleic acid encoding RBD (SEQ ID NO:12)
      Sequences according to embodiments of the invention: CoV_T2_1 (Wuhan_Node1), CoV_T2_4 (Wuhan_Node1_tr), or CoV_T2_7 (Wuhan_Node1_RBD):
Wuhan_Node1 (CoV_T2_1):
   full length S-protein (SEQ ID NO:13) and nucleic acid encoding full length S-protein (SEQ ID NO:14)
Wuhan_Node1_tr (CoV_T2_4):
   truncated S-protein (SEQ ID NO:15) and nucleic acid encoding truncated S-protein (SEQ ID NO:16)
Wuhan_Node1_RBD (COV_T2_7):
   RBD (SEQ ID NO:17) and nucleic acid encoding RBD (SEQ ID NO:18)

>AY274119(COV_T1_1) (SEQ ID NO: 1)
Amino acid sequence:
MFIFLLFLTLTSGSDLDRCTTFDDVQAPNYTQHTSSMRGVYYPDEIFRSDTLYLTQDLFLPFYSNVTG
FHTINHTFGNPVIPFKDGIYFAATEKSNVVRGWVFGSTMNNKSQSVIIINNSTNVVIRACNFELCDNP
FFAVSKPMGTQTHTMIFDNAFNCTFEYISDAFSLDVSEKSGNFKHLREFVEKNKDGFLYVYKGYQPID
VVRDLPSGENTLKPIFKLPLGINITNFRAILTAFSPAQDIWGTSAAAYFVGYLKPTTEMLKYDENGTI
TDAVDCSQNPLAELKCSVKSFEIDKGIYQTSNERVVPSGDVVRFPNITNLCPFGEVENATKEPSVYAW
ERKKISNCVADYSVLYNSTFFSTFKCYGVSATKLNDLCFSNVYADSFVVKGDDVRQIAPGQTGVIADY
NYKLPDDEMGCVLAWNTRNIDATSTGNYNYKYRYLRHGKLRPFERDISNVPFSPDGKPCTPPALNCYW
PLNDYGFYTTTGIGYQPYRVVVLSFELLNAPATVCGPKLSTDLIKNQCVNENENGLTGTGVLTPSSKR
FQPFQQFGRDVSDFTDSVRDPKTSEILDISPCAFGGVSVITPGTNASSEVAVLYQDVNCTDVSTAIHA
DQLTPAWRIYSTGNNVFQTQAGCLIGAEHVDTSYECDIPIGAGICASYHTVSLLRSTSQKSIVAYTMS
LGADSSIAYSNNTIAIPTNESISITTEVMPVSMAKTSVDCNMYICGDSTECANLLLQYGSFCTQLNRA
LSGIAAEQDRNTREVFAQVKQMYKTPTLKYFGGENFSQILPDPLKPTKRSFIEDLLENKVTLADAGFM
KQYGECLGDINARDLICAQKENGLTVLPPLLTDDMIAAYTAALVSGTATAGWTFGAGAALQIPFAMQM
AYRFNGIGVTQNVLYENQKQIANQFNKAISQIQESLTTTSTALGKLQDVVNQNAQALNTLVKQLSSNF
GAISSVLNDILSRLDKVEAEVQIDRLITGRLQSLQTYVTQQLIRAAEIRASANLAATKMSECVLGQSK
RVDFCGKGYHLMSFPQAAPHGVVFLHVTYVPSQERNETTAPAICHEGKAYFPREGVEVENGTSWFITQ
RNFFSPQIITTDNTFVSGNCDVVIGIINNTVYDPLQPELDSFKEELDKYFKNHTSPDVDLGDISGINA
SVVNIQKEIDRLNEVAKNLNESLIDLQELGKYEQYIKWPWYVWLGFIAGLIAIVMVTILLCCMTSCCS
CLKGACSCGSCCKFDEDDSEPVLKGVKLHYT
>AY274119(COV_T1_1) (SEQ ID NO: 2)
Nucleic acid sequence:
atgtttatctttctgctgtttctgaccctgaccagcggcagcgacctggatagatgcacc
accttcgacgatgtgcaggcccctaactacacccagcacaccagctctatgcggggcgtg
tactaccccgacgagattttcagaagcgacaccctgtatctgacccaggacctgttcctg
cctttctacagcaacgtgaccggcttccacaccatcaaccacaccttcggcaaccctgtg
atccccttcaaggacggcatctactttgccgccaccgagaagtccaacgtcgtcagagga
tgggtgttcggcagcaccatgaacaacaagagccagagcgtgatcatcatcaacaacagc
accaacgtggtcatccgggcctgcaacttcgagctgtgcgacaacccattcttcgccgtg
tccaagcctatgggcacccagacacacaccatgatcttcgacaacgccttcaactgcacc
ttcgagtacatcagcgacgccttcagcctggacgtgtccgaaaagagcggcaacttcaag
cacctgagggaattcgtgttcaagaacaaggatggcttcctgtacgtgtacaagggctac
cagcctatcgacgtcgtgcgggatctgcccagcggcttcaataccctgaagcctatcttc
aagctgcccctgggcatcaacatcaccaacttcagagccatcctgaccgctttcagcccc
gctcaggatatctggggaacaagcgccgctgcctacttcgtgggctacctgaagccaacc
accttcatgctgaagtacgacgagaacggcaccatcaccgacgccgtggactgtagccaa
aatcctctggccgagctgaagtgcagcgtgaagtccttcgagatcgacaagggcatctac
cagaccagcaatttcagagtggtgccctccggggatgtcgtgcggttccccaacatcaca
aatctgtgccccttcggcgaggtgttcaacgccaccaagtttccctctgtgtacgcctgg
gagcgcaaaaagatcagcaactgcgtggccgactacagcgtgctgtacaactccaccttc
ttcagcaccttcaagtgctacggcgtgtccgccacaaagctgaacgacctgtgcttctcc
aacgtgtacgccgacagcttcgtggtcaaaggcgacgacgttcggcagattgcccctgga
caaacaggcgtgatcgccgattacaactacaagctgcctgacgacttcatgggctgcgtg
ctggcctggaacaccagaaacatcgatgccacctccaccggcaactacaattacaagtac
agatacctgcggcacggcaagctgcggcctttcgagagggatatcagcaatgtgcctttt
agccccgacggcaagccctgcacacctcctgctctgaattgctactggcccctgaacgac
tacggcttttacaccaccacaggcatcggctatcagccctatagagtggtggtcctgtcc
tttgagctgctgaatgcccctgccacagtgtgcggacctaagctgtctaccgacctgatc
aagaaccagtgcgtgaacttcaacttcaacggcctgaccggcaccggcgtgctgacacca
agcagcaagagattccagcctttccagcagttcggccgggatgtgtccgacttcacagac
agcgtcagagatcccaagaccagcgagatcctggacatcagcccttgtgcctttggcgga
gtgtccgtgatcacccctggcacaaatgcctctagcgaagtggccgtgctgtatcaggac
gtgaactgcaccgatgtgtccaccgccattcacgccgatcagctgactcccgcttggcgg
atctatagcacaggcaacaacgtgttccagacacaagccggctgtctgatcggagccgag
catgtggataccagctacgagtgcgacatccctatcggcgctggcatctgtgcctcttac
cacaccgtgtctctgctgcggagcaccagccagaaatccatcgtggcctacaccatgagc
ctgggcgccgattcttctatcgcctactccaacaacacaatcgctatccccaccaatttc
agcatctccatcaccaccgaagtgatgcccgtgtccatggccaagacctccgtggattgc
aacatgtacatctgcggcgacagcaccgagtgcgccaatctgctgctccagtacggcagc
ttctgcacccagctgaatagagccctgtctggaattgccgccgagcaggacagaaacacc
agagaagtgttcgcccaagtgaagcagatgtataagaccccgacactcaagtacttcggc
gggttcaacttctcccagatcctgcctgatcctctgaagcccaccaagcggagcttcatc
gaggacctgctgttcaacaaagtgaccctggccgacgccggctttatgaagcagtatggc
gagtgcctgggcgacatcaacgccagggatctgatttgcgcccagaagtttaacggactg
accgtgctgcctcctctgctgaccgatgatatgatcgccgcctacacagccgctctggtg
tctggtacagctaccgccggatggacatttggagctggcgccgctctccagattccattc
gctatgcagatggcctaccggttcaacggcatcggagtgacccagaatgtgctgtacgag
aatcagaagcagatcgccaatcagttcaacaaggccatcagccagatccaagagagcctg
accaccacaagcacagccctgggaaagctccaggacgtggtcaaccagaatgctcaggcc
ctgaacaccctggtcaagcagctgagcagcaacttcggcgccatcagctccgtgctgaat
gacatcctgagccggctggacaaggtggaagcagaggtgcagatcgaccggctgatcaca
ggcagactccagagcctccagacctacgtgacacagcagctgatcagagccgccgagatt
agagcctctgccaatctggccgccaccaaaatgagcgagtgtgtcctgggccagagcaag
agagtggacttttgcggcaagggctatcacctgatgagcttcccacaggccgctcctcat
ggcgtggtctttctgcacgtgacatacgtgcccagccaagagagaaacttcaccaccgct
ccagccatctgccacgagggcaaagcctactttcccagagaaggcgtgttcgtgtttaac
ggcacctcctggtttatcacccagcggaatttcttcagcccgcaaatcatcaccacagac
aacaccttcgtgtccggcaactgtgacgtcgtgatcggcatcattaacaataccgtgtac
gaccctctccagcctgagctggacagcttcaaagaggaactggataagtacttcaagaat
cacacgagccccgatgtggacctgggcgatatctctggcatcaatgccagcgtcgtgaac
atccagaaagagattgacaggctgaacgaggtggccaagaacctgaacgagtccctgatc
gacctgcaagagctggggaagtacgagcagtacatcaagtggccttggtacgtgtggctg
ggctttatcgccggactgatcgccatcgtgatggtcaccatcctgctgtgctgcatgacc
agctgttgcagctgtctgaagggcgcctgtagctgtggctcctgctgcaagttcgatgag
gacgactctgagccagtgctgaaaggcgtgaagctgcactacacc
>AY274119_tr(COV_T2_2) (SEQ ID NO: 3)
Amino acid sequence:
MFIFLLFLTLTSGSDLDRCTTFDDVQAPNYTQHTSSMRGVYYPDEIFRSDTLYLTQDLFLPFYSNVTG
FHTINHTFGNPVIPFKDGIYFAATEKSNVVRGWVFGSTMNNKSQSVIIINNSTNVVIRACNFELCDNP
FFAVSKPMGTQTHTMIFDNAFNCTFEYISDAFSLDVSEKSGNFKHLREFVEKNKDGFLYVYKGYQPID
VVRDLPSGENTLKPIFKLPLGINITNFRAILTAFSPAQDIWGTSAAAYFVGYLKPTTEMLKYDENGTI
TDAVDCSQNPLAELKCSVKSFEIDKGIYQTSNFRVVPSGDVVRFPNITNLCPFGEVENATKEPSVYAW
ERKKISNCVADYSVLYNSTFFSTFKCYGVSATKLNDLCFSNVYADSFVVKGDDVRQIAPGQTGVIADY
NYKLPDDFMGCVLAWNTRNIDATSTGNYNYKYRYLRHGKLRPFERDISNVPFSPDGKPCTPPALNCYW
PLNDYGFYTTTGIGYQPYRVVVLSFELLNAPATVCGPKLSTDLIKNQCVNENENGLTGTGVLTPSSKR
FQPFQQFGRDVSDFTDSVRDPKTSEILDISPCAFGGVSVITPGTNASSEVAVLYQDVNCTDVSTAIHA
DQLTPAWRIYSTGNNVFQTQAGCLIGAEHVDTSYECDIPIGAGICASYHTVSLLRSTSQKSIVAYTMS
LGADSSIAYSNNTIAIPTNFSISITTEVMPVSMAKTSVDCNMYICGDSTECANLLLQYGSFCTQLNRA
LSGIAAEQDRNTREVFAQVKQMYKTPTLKYFGGFNFSQILPDPLKPTKRSFIEDLLENKVTLADAGfM
KQYGECLGDINARDLICAQKENGLTVLPPLLTDDMIAAYTAALVSGTATAGWTFGAGAALQIPFAMQM
AYRENGIGVTQNVLYENQKQIANQFNKAISQIQESLTTTSTALGKLQDVVNQNAQALNTLVKQLSSNF
GAISSVLNDILSRLDKVEAEVQIDRLITGRLQSLQTYVTQQLIRAAEIRASANLAATKMSECVLGQSK
RVDFCGKGYHLMSFPQAAPHGVVFLHVTYVPSQERNFTTAPAICHEGKAYFPREGVEVENGTSWFITQ
RNFFSPQIITTDNTFVSGNCDVVIGIINNTVYDPLQPELDSFKEELDKYFKNHTSPDVDLGDIS
>AY274119_tr(COV_T2_2) (SEQ ID NO: 4)
Nucleic acid sequence:
atgtttatctttctgctgtttctgaccctgaccagcggcagcgacctggatagatgcacc
accttcgacgatgtgcaggcccctaactacacccagcacaccagctctatgcggggcgtg
tactaccccgacgagattttcagaagcgacaccctgtatctgacccaggacctgttcctg
cctttctacagcaacgtgaccggcttccacaccatcaaccacaccttcggcaaccctgtg
atccccttcaaggacggcatctactttgccgccaccgagaagtccaacgtcgtcagagga
tgggtgttcggcagcaccatgaacaacaagagccagagcgtgatcatcatcaacaacagc
accaacgtggtcatccgggcctgcaacttcgagctgtgcgacaacccattcttcgccgtg
tccaagcctatgggcacccagacacacaccatgatcttcgacaacgccttcaactgcacc
ttcgagtacatcagcgacgccttcagcctggacgtgtccgaaaagagcggcaacttcaag
cacctgagggaattcgtgttcaagaacaaggatggcttcctgtacgtgtacaagggctac
cagcctatcgacgtcgtgcgggatctgcccagcggcttcaataccctgaagcctatcttc
aagctgcccctgggcatcaacatcaccaacttcagagccatcctgaccgctttcagcccc
gctcaggatatctggggaacaagcgccgctgcctacttcgtgggctacctgaagccaacc
accttcatgctgaagtacgacgagaacggcaccatcaccgacgccgtggactgtagccaa
aatcctctggccgagctgaagtgcagcgtgaagtccttcgagatcgacaagggcatctac
cagaccagcaatttcagagtggtgccctccggggatgtcgtgcggttccccaacatcaca
aatctgtgccccttcggcgaggtgttcaacgccaccaagtttccctctgtgtacgcctgg
gagcgcaaaaagatcagcaactgcgtggccgactacagcgtgctgtacaactccaccttc
ttcagcaccttcaagtgctacggcgtgtccgccacaaagctgaacgacctgtgcttctcc
aacgtgtacgccgacagcttcgtggtcaaaggcgacgacgttcggcagattgcccctgga
caaacaggcgtgatcgccgattacaactacaagctgcctgacgacttcatgggctgcgtg
ctggcctggaacaccagaaacatcgatgccacctccaccggcaactacaattacaagtac
agatacctgcggcacggcaagctgcggcctttcgagagggatatcagcaatgtgcctttt
agccccgacggcaagccctgcacacctcctgctctgaattgctactggcccctgaacgac
tacggcttttacaccaccacaggcatcggctatcagccctatagagtggtggtcctgtcc
tttgagctgctgaatgcccctgccacagtgtgcggacctaagctgtctaccgacctgatc
aagaaccagtgcgtgaacttcaacttcaacggcctgaccggcaccggcgtgctgacacca
agcagcaagagattccagcctttccagcagttcggccgggatgtgtccgacttcacagac
agcgtcagagatcccaagaccagcgagatcctggacatcagcccttgtgcctttggcgga
gtgtccgtgatcacccctggcacaaatgcctctagcgaagtggccgtgctgtatcaggac
gtgaactgcaccgatgtgtccaccgccattcacgccgatcagctgactcccgcttggcgg
atctatagcacaggcaacaacgtgttccagacacaagccggctgtctgatcggagccgag
catgtggataccagctacgagtgcgacatccctatcggcgctggcatctgtgcctcttac
cacaccgtgtctctgctgcggagcaccagccagaaatccatcgtggcctacaccatgagc
ctgggcgccgattcttctatcgcctactccaacaacacaatcgctatccccaccaatttc
agcatctccatcaccaccgaagtgatgcccgtgtccatggccaagacctccgtggattgc
aacatgtacatctgcggcgacagcaccgagtgcgccaatctgctgctccagtacggcagc
ttctgcacccagctgaatagagccctgtctggaattgccgccgagcaggacagaaacacc
agagaagtgttcgcccaagtgaagcagatgtataagaccccgacactcaagtacttcggc
gggttcaacttctcccagatcctgcctgatcctctgaagcccaccaagcggagcttcatc
gaggacctgctgttcaacaaagtgaccctggccgacgccggctttatgaagcagtatggc
gagtgcctgggcgacatcaacgccagggatctgatttgcgcccagaagtttaacggactg
accgtgctgcctcctctgctgaccgatgatatgatcgccgcctacacagccgctctggtg
tctggtacagctaccgccggatggacatttggagctggcgccgctctccagattccattc
gctatgcagatggcctaccggttcaacggcatcggagtgacccagaatgtgctgtacgag
aatcagaagcagatcgccaatcagttcaacaaggccatcagccagatccaagagagcctg
accaccacaagcacagccctgggaaagctccaggacgtggtcaaccagaatgctcaggcc
ctgaacaccctggtcaagcagctgagcagcaacttcggcgccatcagctccgtgctgaat
gacatcctgagccggctggacaaggtggaagcagaggtgcagatcgaccggctgatcaca
ggcagactccagagcctccagacctacgtgacacagcagctgatcagagccgccgagatt
agagcctctgccaatctggccgccaccaaaatgagcgagtgtgtcctgggccagagcaag
agagtggacttttgcggcaagggctatcacctgatgagcttcccacaggccgctcctcat
ggcgtggtctttctgcacgtgacatacgtgcccagccaagagagaaacttcaccaccgct
ccagccatctgccacgagggcaaagcctactttcccagagaaggcgtgttcgtgtttaac
ggcacctcctggtttatcacccagcggaatttcttcagcccgcaaatcatcaccacagac
aacaccttcgtgtccggcaactgtgacgtcgtgatcggcatcattaacaataccgtgtac
gaccctctccagcctgagctggacagcttcaaagaggaactggataagtacttcaagaat
cacacgagccccgatgtggacctgggcgatatctct
>AY274119_RBD (COV_T2_5) (SEQ ID NO: 5)
Amino acid sequence:
RVVPSGDVVRFPNITNLCPFGEVENATKEPSVYAWERKKISNCVADYSVLYNSTFFSTEKCYGVSATK
LNDLCFSNVYADSFVVKGDDVRQIAPGQTGVIADYNYKLPDDFMGCVLAWNTRNIDATSTGNYNYKYR
YLRHGKLRPFERDISNVPFSPDGKPCTPPALNCYWPLNDYGFYTTTGIGYQPYRVVVLSFELLNAPAT
VCGPKLSTD
>AY274119_RBD (COV_T2 5) (SEQ ID NO: 6)
Nucleic acid sequence:
agagtggtgccctccggggatgtcgtgcggttccccaacatcacaaatctgtgccccttc
ggcgaggtgttcaacgccaccaagtttccctctgtgtacgcctgggagcgcaaaaagatc
agcaactgcgtggccgactacagcgtgctgtacaactccaccttcttcagcaccttcaag
tgctacggcgtgtccgccacaaagctgaacgacctgtgcttctccaacgtgtacgccgac
agcttcgtggtcaaaggcgacgacgttcggcagattgcccctggacaaacaggcgtgatc
gccgattacaactacaagctgcctgacgacttcatgggctgcgtgctggcctggaacacc
agaaacatcgatgccacctccaccggcaactacaattacaagtacagatacctgcggcac
ggcaagctgcggcctttcgagagggatatcagcaatgtgccttttagccccgacggcaag
ccctgcacacctcctgctctgaattgctactggcccctgaacgactacggcttttacacc
accacaggcatcggctatcagccctatagagtggtggtcctgtcctttgagctgctgaat
gcccctgccacagtgtgcggacctaagctgtctaccgac
AY274119 (full length S protein amino acid sequence, with RBD
residues shown in bold, and residues not present in truncated S
protein shown underlined) (SEQ ID NO: 1)

| | |
|---|---|
| MFIFLLFLTL TSGSDLDRCT TFDDVQAPNY TQHTSSMRGV YYPDEIFRSD TLYLTQDLFL | 60 |
| PFYSNVTGFH TINHTFGNPV IPFKDGIYFA ATEKSNVVRG WVFGSTMNNK SQSVIIINNS | 120 |
| TNVVIRACNF ELCDNPFFAV SKPMGTQTHT MIFDNAFNCT FEYISDAFSL DVSEKSGNFK | 180 |
| HLREFVFKNK DGFLYVYKGY QPIDVVRDLP SGFNTLKPIF KLPLGINITN FRAILTAFSP | 240 |
| AQDIWGTSAA AYFVGYLKPT TFMLKYDENG TITDAVDCSQ NPLAELKCSV KSFEIDKGIY | 300 |
| QTSNFRVVPS GDVVRFPNIT NLCPFGEVFN ATKFPSVYAW ERKKISNCVA DYSVLYNSTF | 360 |
| FSTFKCYGVS ATKLNDLCFS NVYADSFVVK GDDVRQIAPG QTGVIADYNY KLPDDFMGCV | 420 |
| LAWNTRNIDA TSTGNYNYKY RYLRHGKLRP FERDISNVPF SPDGKPCTPP ALNCYWPLND | 480 |
| YGFYTTTGIG YQPYRVVVLS FELLNAPATV CGPKLSTDLI KNQCVNFNFN GLTGTGVLTP | 540 |
| SSKRFQPFQQ FGRDVSDFTD SVRDPKTSEI LDISPCAFGG VSVITPGTNA SSEVAVLYQD | 600 |
| VNCTDVSTAI HADQLTPAWR IYSTGNNVFQ TQAGCLIGAE HVDTSYECDI PIGAGICASY | 660 |
| HTVSLLRSTS QKSIVAYTMS LGADSSIAYS NNTIAIPTNF SISITTEVMP VSMAKTSVDC | 720 |
| NMYICGDSTE CANLLLQYGS FCTQLNRALS GIAAEQDRNT REVFAQVKQM YKTPTLKYFG | 780 |
| GFNFSQILPD PLKPTKRSFI EDLLFNKVTL ADAGFMKQYG ECLGDINARD LICAQKFNGL | 840 |
| TVLPPLLTDD MIAAYTAALV SGTATAGWTF GAGAALQIPF AMQMAYRFNG IGVTQNVLYE | 900 |
| NQKQIANQFN KAISQIQESL TTTSTALGKL QDVVNQNAQA LNTLVKQLSS NFGAISSVLN | 960 |
| DILSRLDKVE AEVQIDRLIT GRLQSLQTYV TQQLIRAAEI RASANLAATK MSECVLGQSK | 1020 |
| RVDFCGKGYH LMSFPQAAPH GVVFLHVTYV PSQERNFTTA PAICHEGKAY FPREGVFVFN | 1080 |
| GTSWFITQRN FFSPQIITTD NTFVSGNCDV VIGIINNTVY DPLQPELDSF KEELDKYFKN | 1140 |
| HTSPDVDLGD ISGINASVVN IQKEIDRLNE VAKNLNESLI DLQELGKYEQ YIKWPWYVWL | 1200 |
| GFIAGLIAIV MVTILLCCMT SCCSCLKGAC SCGSCCKFDE DDSEPVLKGV KLHYT | 1255 |

>EPI_ISL_402119 (COV_T1 2) (SEQ ID NO: 7)
Amino acid sequence:
MFVFLVLLPLVSSQCVNLTTRTQLPPAYTNSFTRGVYYPDKVERSSVLHSTQDLFLPFFSNVTWFHAI
HVSGTNGTKRFDNPVLPENDGVYFASTEKSNIIRGWIFGTTLDSKTQSLLIVNNATNVVIKVCEFQFC
NDPFLGVYYHKNNKSWMESEFRVYSSANNCTFEYVSQPFLMDLEGKQGNEKNLREFVFKNIDGYFKIY
SKHTPINLVRDLPQGFSALEPLVDLPIGINITRFQTLLALHRSYLTPGDSSSGWTAGAAAYYVGYLQP
RTFLLKYNENGTITDAVDCALDPLSETKCTLKSFTVEKGIYQTSNERVQPTESIVRFPNITNLCPFGE
VFNATRFASVYAWNRKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLERKSNLKPFERDISTEIYQAG
STPCNGVEGENCYFPLQSYGFQPTNGVGYQPYRVVVLSFELLHAPATVCGPKKSTNLVKNKCVNFNFN
GLTGTGVLTESNKKELPFQQFGRDIADTTDAVRDPQTLEILDITPCSFGGVSVITPGTNTSNQVAVLY
QDVNCTEVPVAIHADQLTPTWRVYSTGSNVFQTRAGCLIGAEHVNNSYECDIPIGAGICASYQTQTNS
PRRARSVASQSIIAYTMSLGAENSVAYSNNSIAIPTNFTISVTTEILPVSMTKTSVDCTMYICGDSTE
CSNLLLQYGSFCTQLNRALTGIAVEQDKNTQEVFAQVKQIYKTPPIKDFGGENFSQILPDPSKPSKRS
FIEDLLENKVTLADAGFIKQYGDCLGDIAARDLICAQKENGLTVLPPLLTDEMIAQYTSALLAGTITS
GWTFGAGAALQIPFAMQMAYRENGIGVTQNVLYENQKLIANQFNSAIGKIQDSLSSTASALGKLQDVV
NQNAQALNTLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFKEELDKYF
KNHTSPDVDLGDISGINASVVNIQKEIDRLNEVAKNLNESLIDLQELGKYEQYIKWPWYIWLGFIAGL
IAIVMVTIMLCCMTSCCSCLKGCCSCGSCCKFDEDDSEPVLKGVKLHYT
>EPI_ISL_402119 (COV_T1_2) (SEQ ID NO: 8)
Nucleic acid sequence:
atgttcgtgtttctggtgctgctgcctctggtgtccagccagtgtgtgaacctgaccacc
agaacacagctgcctccagcctacaccaacagctttaccagaggcgtgtactaccccgac
aaggtgttcagatccagcgtgctgcactctacccaggacctgttcctgcctttcttcagc
aacgtgacctggttccacgccatccacgtgtccggcaccaatggcaccaagagattcgac
aaccccgtgctgcccttcaacgacggggtgtactttgccagcaccgagaagtccaacatc
atcagaggctggatcttcggcaccacactggacagcaagacccagagcctgctgatcgtg
aacaacgccaccaacgtggtcatcaaagtgtgcgagttccagttctgcaacgaccccttc
ctgggcgtctactaccacaagaacaacaagagctggatggaaagcgagttccgggtgtac
agcagcgccaacaactgcaccttcgagtacgtgtcccagcctttcctgatggacctggaa
ggcaagcagggcaacttcaagaacctgcgcgagttcgtgttcaagaacatcgacggctac
ttcaaaatctacagcaagcacacccctatcaacctcgtgcgggatctgcctcagggcttc
tctgctctggaacccctggtggatctgcccatcggcatcaacatcacccggtttcagaca
ctgctggccctgcacagaagctacctgacacctggcgatagcagcagcggatggacagct
ggtgccgccgcttactacgtgggatacctccagccaagaaccttcctgctgaagtacaac
gagaacggcaccatcaccgacgccgtggattgtgctctggaccctctgagcgagacaaag
tgcaccctgaagtccttcaccgtggaaaagggcatctaccagaccagcaacttccgggtg
cagcccaccgaatccatcgtgcggttccccaatatcaccaatctgtgccccttcggcgag
gtgttcaatgccaccagattcgcctctgtgtacgcctggaaccggaagcggatcagcaat
tgcgtggccgactactccgtgctgtacaactccgccagcttcagcaccttcaagtgctac
ggcgtgtcccctaccaagctgaacgacctgtgcttcacaaacgtgtacgccgacagcttc
gtgatccggggagatgaagtgcggcagattgcccctggacagacaggcaagatcgccgac
tacaactacaagctgcccgacgacttcaccggctgtgtgattgcctggaacagcaacaac
ctggactccaaagtcggcggcaactacaattacctgtaccggctgttccggaagtccaat
ctgaagcccttcgagcgggacatcagcaccgaaatctatcaggccggcagcaccccttgc
aacggcgtggaaggcttcaactgctacttcccactgcaaagctacggctttcagcccaca
aatggcgtgggctaccagccttacagagtggtggtgctgagcttcgagctgctgcatgct
cctgccacagtgtgcggccctaagaaatccaccaatctcgtgaagaacaaatgcgtgaac
ttcaacttcaacggcctgaccggcaccggcgtgctgacagagagcaacaagaagttcctg
ccattccagcagttcggccgggatatcgccgataccacagatgccgtcagagatccccag
acactggaaatcctggacatcaccccatgcagcttcggcggagtgtctgtgatcacccct
ggcaccaacaccagcaatcaggtggcagtgctgtaccaggacgtgaactgtaccgaagtg
cccgtggccattcacgccgatcagctgacacctacatggcgggtgtactccaccggcagc
aatgtgtttcagaccagagccggctgtctgatcggagccgagcacgtgaacaatagctac
gagtgcgacatccccatcggcgctggcatctgcgcctcttaccagacacagacaaacagc
cccagacgggctagaagcgtggccagccagagcatcattgcctacacaatgtctctgggc
gccgagaacagcgtggcctactccaacaactctatcgctatccccaccaacttcaccatc
agcgtgaccaccgagatcctgcctgtgtccatgaccaagaccagcgtggactgcaccatg
tacatctgcggcgattccaccgagtgctccaacctgctgctccagtacggcagcttctgc
acccagctgaatagagccctgacagggatcgccgtggaacaggacaagaacacccaagag
gtgttcgcccaagtgaagcaaatctacaagacccctcctatcaaggacttcggcggcttc
aatttcagccagattctgcccgatcctagcaagcccagcaagcggagcttcatcgaggac
ctgctgttcaacaaagtgacactggccgacgccggcttcatcaagcagtacggcgattgt
ctgggcgacattgccgccagggatctgatttgcgcccagaagtttaacggactgacagtg
ctgcctcctctgctgaccgatgagatgatcgcccagtacacatctgccctgctggccggc
acaatcacaagcggctggacatttggagctggcgccgctctccagattccattcgctatg
cagatggcctaccggttcaacggcatcggagtgacccagaatgtgctgtacgagaaccag
aagctgatcgccaaccagttcaacagcgccatcggcaagatccaggacagcctgagcagc
acagcaagcgccctgggaaagctccaggacgtcgtgaaccagaatgcccaggcactgaac
accctggtcaagcagctgtcctccaacttcggcgccatcagctctgtgctgaacgatatc
ctgagcagactggacaaggtggaagccgaggtgcagatcgacagactgatcaccggcaga
ctccagtctctccagacctacgtgacccagcagctgatcagagccgccgagattagagcc
tctgccaatctggccgccaccaagatgtctgagtgtgtgctgggccagagcaagagagtg
gacttttgcggcaagggctaccacctgatgagcttccctcagtctgcccctcacggcgtg
gtgtttctgcacgtgacatacgtgcccgctcaagagaagaatttcaccaccgctccagcc
atctgccacgacggcaaagcccactttcctagagaaggcgtgttcgtgtccaacggcacc
cattggttcgtgacacagcggaacttctacgagccccagatcatcaccaccgacaacacc
ttcgtgtctggcaactgcgacgttgtgatcggcattgtgaacaataccgtgtacgaccct
ctccagcctgaactggactccttcaaagaggaactcgacaagtactttaagaaccacaca
agccccgacgtggacctgggcgatatcagcggaatcaatgccagcgtggtcaacatccag
aaagagatcgaccggctgaacgaggtggccaagaatctgaacgagagcctgatcgacctg
caagaactggggaagtacgagcagtacatcaagtggccctggtacatctggctgggcttt
atcgccggactgattgccatcgtgatggtcacaatcatgctgtgttgcatgaccagctgc
tgtagctgcctgaagggctgttgtagctgtggctcctgctgcaagttcgacgaggacgat
tctgagcccgtgctgaagggcgtgaaactgcactacacc
>EPI_ISL_402119_tr (COV_T2_3) (SEQ ID NO: 9)
Amino acid sequence:
MFVFLVLLPLVSSQCVNLTTRTQLPPAYTNSFTRGVYYPDKVFRSSVLHSTQDLFLPFFSNVTWFHAI
HVSGTNGTKRFDNPVLPENDGVYFASTEKSNIIRGWIFGTTLDSKTQSLLIVNNATNVVIKVCEFQFC
NDPFLGVYYHKNNKSWMESEFRVYSSANNCTFEYVSQPFLMDLEGKQGNEKNLREFVFKNIDGYFKIY
SKHTPINLVRDLPQGFSALEPLVDLPIGINITRFQTLLALHRSYLTPGDSSSGWTAGAAAYYVGYLQP
RTFLLKYNENGTITDAVDCALDPLSETKCTLKSFTVEKGIYQTSNERVQPTESIVRFPNITNLCPFGE
VFNATRFASVYAWNRKRISNCVADYSVLYNSASFSTFKCYGVSPTKLNDLCFTNVYADSEVIRGDEVR
QIAPGQTGKIADYNYKLPDDFTGCVIAWNSNNLDSKVGGNYNYLYRLFRKSNLKPFERDISTEIYQAG
STPCNGVEGFNCYFPLQSYGFQPTNGVGYQPYRVVVLSFELLHAPATVCGPKKSTNLVKNKCVNFNFN
GLTGTGVLTESNKKELPFQQFGRDIADTTDAVRDPQTLEILDITPCSFGGVSVITPGTNTSNQVAVLY
QDVNCTEVPVAIHADQLTPTWRVYSTGSNVFQTRAGCLIGAEHVNNSYECDIPIGAGICASYQTQTNS
PRRARSVASQSIIAYTMSLGAENSVAYSNNSIAIPTNFTISVTTEILPVSMTKTSVDCTMYICGDSTE
CSNLLLQYGSFCTQLNRALTGIAVEQDKNTQEVFAQVKQIYKTPPIKDFGGENFSQILPDPSKPSKRS
FIEDLLENKVTLADAGFIKQYGDCLGDIAARDLICAQKENGLTVLPPLLTDEMIAQYTSALLAGTITS
GWTFGAGAALQIPFAMQMAYRENGIGVTQNVLYENQKLIANQFNSAIGKIQDSLSSTASALGKLQDVV
NQNAQALNTLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFKEELDKYF
KNHTSPDVDLGDIS
>EPI_ISL_402119_tr (COV_T2_3) (SEQ ID NO: 10)
Nucleic acid sequence:
atgttcgtgtttctggtgctgctgcctctggtgtccagccagtgtgtgaacctgaccacc
agaacacagctgcctccagcctacaccaacagctttaccagaggcgtgtactaccccgac
aaggtgttcagatccagcgtgctgcactctacccaggacctgttcctgcctttcttcagc
aacgtgacctggttccacgccatccacgtgtccggcaccaatggcaccaagagattcgac
aaccccgtgctgcccttcaacgacggggtgtactttgccagcaccgagaagtccaacatc
atcagaggctggatcttcggcaccacactggacagcaagacccagagcctgctgatcgtg
aacaacgccaccaacgtggtcatcaaagtgtgcgagttccagttctgcaacgaccccttc
ctgggcgtctactaccacaagaacaacaagagctggatggaaagcgagttccgggtgtac
agcagcgccaacaactgcaccttcgagtacgtgtcccagcctttcctgatggacctggaa
ggcaagcagggcaacttcaagaacctgcgcgagttcgtgttcaagaacatcgacggctac
ttcaaaatctacagcaagcacacccctatcaacctcgtgcgggatctgcctcagggcttc
tctgctctggaacccctggtggatctgcccatcggcatcaacatcacccggtttcagaca
ctgctggccctgcacagaagctacctgacacctggcgatagcagcagcggatggacagct
ggtgccgccgcttactacgtgggatacctccagccaagaaccttcctgctgaagtacaac
gagaacggcaccatcaccgacgccgtggattgtgctctggaccctctgagcgagacaaag
tgcaccctgaagtccttcaccgtggaaaagggcatctaccagaccagcaacttccgggtg
cagcccaccgaatccatcgtgcggttccccaatatcaccaatctgtgccccttcggcgag
gtgttcaatgccaccagattcgcctctgtgtacgcctggaaccggaagcggatcagcaat
tgcgtggccgactactccgtgctgtacaactccgccagcttcagcaccttcaagtgctac
ggcgtgtcccctaccaagctgaacgacctgtgcttcacaaacgtgtacgccgacagcttc
gtgatccggggagatgaagtgcggcagattgcccctggacagacaggcaagatcgccgac
tacaactacaagctgcccgacgacttcaccggctgtgtgattgcctggaacagcaacaac
ctggactccaaagtcggcggcaactacaattacctgtaccggctgttccggaagtccaat
ctgaagcccttcgagcgggacatcagcaccgaaatctatcaggccggcagcaccccttgc
aacggcgtggaaggcttcaactgctacttcccactgcaaagctacggctttcagcccaca
aatggcgtgggctaccagccttacagagtggtggtgctgagcttcgagctgctgcatgct
cctgccacagtgtgcggccctaagaaatccaccaatctcgtgaagaacaaatgcgtgaac
ttcaacttcaacggcctgaccggcaccggcgtgctgacagagagcaacaagaagttcctg
ccattccagcagttcggccgggatatcgccgataccacagatgccgtcagagatccccag
acactggaaatcctggacatcaccccatgcagcttcggcggagtgtctgtgatcacccct
ggcaccaacaccagcaatcaggtggcagtgctgtaccaggacgtgaactgtaccgaagtg
cccgtggccattcacgccgatcagctgacacctacatggcgggtgtactccaccggcagc
aatgtgtttcagaccagagccggctgtctgatcggagccgagcacgtgaacaatagctac
gagtgcgacatccccatcggcgctggcatctgcgcctcttaccagacacagacaaacagc
cccagacgggctagaagcgtggccagccagagcatcattgcctacacaatgtctctgggc
gccgagaacagcgtggcctactccaacaactctatcgctatccccaccaacttcaccatc
agcgtgaccaccgagatcctgcctgtgtccatgaccaagaccagcgtggactgcaccatg
tacatctgcggcgattccaccgagtgctccaacctgctgctccagtacggcagcttctgc
acccagctgaatagagccctgacagggatcgccgtggaacaggacaagaacacccaagag
gtgttcgcccaagtgaagcaaatctacaagacccctcctatcaaggacttcggcggcttc
aatttcagccagattctgcccgatcctagcaagcccagcaagcggagcttcatcgaggac
ctgctgttcaacaaagtgacactggccgacgccggcttcatcaagcagtacggcgattgt
ctgggcgacattgccgccagggatctgatttgcgcccagaagtttaacggactgacagtg
ctgcctcctctgctgaccgatgagatgatcgcccagtacacatctgccctgctggccggc
acaatcacaagcggctggacatttggagctggcgccgctctccagattccattcgctatg
cagatggcctaccggttcaacggcatcggagtgacccagaatgtgctgtacgagaaccag
aagctgatcgccaaccagttcaacagcgccatcggcaagatccaggacagcctgagcagc
acagcaagcgccctgggaaagctccaggacgtcgtgaaccagaatgcccaggcactgaac
accctggtcaagcagctgtcctccaacttcggcgccatcagctctgtgctgaacgatatc
ctgagcagactggacaaggtggaagccgaggtgcagatcgacagactgatcaccggcaga
ctccagtctctccagacctacgtgacccagcagctgatcagagccgccgagattagagcc
tctgccaatctggccgccaccaagatgtctgagtgtgtgctgggccagagcaagagagtg
gacttttgcggcaagggctaccacctgatgagcttccctcagtctgcccctcacggcgtg
gtgtttctgcacgtgacatacgtgcccgctcaagagaagaatttcaccaccgctccagcc
atctgccacgacggcaaagcccactttcctagagaaggcgtgttcgtgtccaacggcacc
cattggttcgtgacacagcggaacttctacgagccccagatcatcaccaccgacaacacc
ttcgtgtctggcaactgcgacgttgtgatcggcattgtgaacaataccgtgtacgaccct
ctccagcctgaactggactccttcaaagaggaactcgacaagtactttaagaaccacaca
agccccgacgtggacctgggcgatatcagt
>EPI_ISL_402119_RBD (COV_T2_6) (SEQ ID NO: 11)
Amino acid sequence:
RVQPTESIVRFPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVGGNYNYLYR
LFRKSNLKPFERDISTEIYQAGSTPCNGVEGENCYFPLQSYGFQPTNGVGYQPYRVVVLSFELLHAPA
TVCGPKKSTN
>EPI_ISL_402119_RBD (COV_T2_6) (SEQ ID NO: 12)
Nucleic acid sequence:
cgggtgcagcccaccgaatccatcgtgcggttccccaatatcaccaatctgtgccccttc
ggcgaggtgttcaatgccaccagattcgcctctgtgtacgcctggaaccggaagcggatc
agcaattgcgtggccgactactccgtgctgtacaactccgccagcttcagcaccttcaag
tgctacggcgtgtcccctaccaagctgaacgacctgtgcttcacaaacgtgtacgccgac
agcttcgtgatccggggagatgaagtgcggcagattgcccctggacagacaggcaagatc
gccgactacaactacaagctgcccgacgacttcaccggctgtgtgattgcctggaacagc
aacaacctggactccaaagtcggcggcaactacaattacctgtaccggctgttccggaag
tccaatctgaagcccttcgagcgggacatcagcaccgaaatctatcaggccggcagcacc
ccttgcaacggcgtggaaggcttcaactgctacttcccactgcaaagctacggctttcag
cccacaaatggcgtgggctaccagccttacagagtggtggtgctgagcttcgagctgctg
catgctcctgccacagtgtgcggccctaagaaatccaccaat
EPI_ISL_402119 (full length S protein amino acid sequence, with RBD
residues shown in bold, and residues not present in truncated S
protein shown underlined) (SEQ ID NO: 7)

| | |
|---|---|
| MFVFLVLLPL VSSQCVNLTT RTQLPPAYTN SFTRGVYYPD KVFRSSVLHS TQDLFLPFFS | 60 |
| NVTWFHAIHV SGTNGTKRFD NPVLPFNDGV YFASTEKSNI IRGWIFGTTL DSKTQSLLIV | 120 |
| NNATNVVIKV CEFQFCNDPF LGVYYHKNNK SWMESEFRVY SSANNCTFEY VSQPFLMDLE | 180 |
| GKQGNFKNLR EFVFKNIDGY FKIYSKHTPI NLVRDLPQGF SALEPLVDLP IGINITRFQT | 240 |
| LLALHRSYLT PGDSSSGWTA GAAAYYVGYL QPRTFLLKYN ENGTITDAVD CALDPLSETK | 300 |
| CTLKSFTVEK GIYQTSNFRV QPTESIVRFP NITNLCPFGE VFNATRFASV YAWNRKRISN | 360 |
| CVADYSVLYN SASFSTFKCY GVSPTKLNDL CFTNVYADSF VIRGDEVRQI APGQTGKIAD | 420 |
| YNYKLPDDFT GCVIAWNSNN LDSKVGGNYN YLYRLFRKSN LKPFERDIST EIYQAGSTPC | 480 |
| NGVEGFNCYF PLQSYGFQPT NGVGYQPYRV VVLSFELLHA PATVCGPKKS TNLVKNKCVN | 540 |
| FNFNGLTGTG VLTESNKKFL PFQQFGRDIA DTTDAVRDPQ TLEILDITPC SFGGVSVITP | 600 |
| GTNTSNQVAV LYQDVNCTEV PVAIHADQLT PTWRVYSTGS NVFQTRAGCL IGAEHVNNSY | 660 |
| ECDIPIGAGI CASYQTQTNS PRRARSVASQ SIIAYTMSLG AENSVAYSNN SIAIPTNFTI | 720 |
| SVTTEILPVS MTKTSVDCTM YICGDSTECS NLLLQYGSFC TQLNRALTGI AVEQDKNTQE | 780 |
| VFAQVKQIYK TPPIKDFGGF NFSQILPDPS KPSKRSFIED LLFNKVTLAD AGFIKQYGDC | 840 |
| LGDIAARDLI CAQKFNGLTV LPPLLTDEMI AQYTSALLAG TITSGWTFGA GAALQIPFAM | 900 |
| QMAYRFNGIG VTQNVLYENQ KLIANQFNSA IGKIQDSLSS TASALGKLQD VVNQNAQALN | 960 |
| TLVKQLSSNF GAISSVLNDI LSRLDKVEAE VQIDRLITGR LQSLQTYVTQ QLIRAAEIRA | 1020 |
| SANLAATKMS ECVLGQSKRV DFCGKGYHLM SFPQSAPHGV VFLHVTYVPA QEKNFTTAPA | 1080 |
| ICHDGKAHFP REGVFVSNGT HWFVTQRNFY EPQIITTDNT FVSGNCDVVI GIVNNTVYDP | 1140 |
| LQPELDSFKE ELDKYFKNHT SPDVDLGDIS GINASVVNIQ KEIDRLNEVA KNLNESLIDL | 1200 |
| QELGKYEQYI KWPWYIWLGF IAGLIAIVMV TIMLCCMTSC CSCLKGCCSC GSCCKFDEDD | 1260 |
| SEPVLKGVKL HYT | 1273 |

>Wuhan_Node1 (COV_T2_1) (SEQ ID NO: 13)
Amino acid sequence:
MFLFLFIIIFAFFLLSAKANERCGIFTSKPQPKLAQVSSSRRGVYYPDDIFRSDVLHLTQDYELPFDS
NVTRYFSLNANGPDRIVYFDNPIIPFKDGVYFAATEKSNVIRGWIFGSTLDNTSQSVIIVNNSTNVII
RVCNFDLCNDPFFTVSRPTDKHIKTWSIREFAVYQSAFNCTFEYVSKSELLDVAEKPGNFKHLREFVF
KNVDGFLNVYSTYKPINVVSGLPTGFSVLKPILKLPLGINITSFRVLLTMERGDPTPGHTTANWLTAA
AAYYVGYLKPTTFMLKYNENGTITDAVDCSQNPLAELKCTLKNENVDKGIYQTSNFRVSPTQEVVRFP
NITNLCPFDKVENATRFPSVYAWERTKISDCVADYTVLYNSTSFSTFKCYGVSPSKLIDLCFTSVYAD
TFLIRCSEVRQVAPGQTGVIADYNYKLPDDFTGCVIAWNTAKQDTGSSGNYNYYYRSHRKTKLKPFER
DLSSDECSPDGKPCTPPAFNGVRGENCYFTLSTYDENPNVPVEYQATRVVVLSFELLNAPATVCGPKL
STQLVKNQCVNFNFNGLKGTGVLTASSKRFQSFQQFGRDASDETDSVRDPQTLEILDISPCSFGGVSV
ITPGTNTSSEVAVLYQDVNCTDVPTAIHADQLTPAWRVYSTGVNVFQTQAGCLIGAEHVNASYECDIP
IGAGICASYHTASNSPRILRSTGQKSIVAYTMSLGAENSIAYANNSIAIPTNESISVTTEVMPVSMAK
TSVDCTMYICGDSLECSNLLLQYGSFCTQLNRALTGIAIEQDKNTQEVFAQVKQMYKTPAIKDEGGFN
FSQILPDPSKPTKRSFIEDLLENKVTLADAGEMKQYGECLGDISARDLICAQKENGLTVLPPLLTDEM
IAAYTAALVSGTATAGWTFGAGAALQIPFAMQMAYRENGIGVTQNVLYENQKQIANQENKAISQIQES
LTTTSTALGKLQDVVNQNAQALNTLVKQLSSNFGAISSVLNDILSRLDKVEAEVQIDRLITGRLQSLQ
TYVTQQLIRAAEIRASANLAATKMSECVLGQSKRVDFCGKGYHLMSFPQAAPHGVVFLHVTYVPSQER
NFTTAPAICHEGKAYFPREGVFVSNGTSWFITQRNFYSPQIITTDNTFVSGNCDVVIGIINNTVYDPL
QPELDSFKEELDKYFKNHTSPDVDLGDISGINASVVNIQKEIDRLNEVAKNLNESLIDLQELGKYEQY
IKWPWYVWLGFIAGLIAIVMATILLCCMTSCCSCLKGACSCGSCCKEDEDDSEPVLKGVKLHYT
>Wuhan_Node1 (COV_T2_1) (SEQ ID NO: 14)
Nucleic acid sequence:
atgtttctgttcctcttcattattatcttcgcattcttcctgctgagcgccaaggccaac
gagagatgcggcatcttcaccagcaagccccagcctaagctggcccaggtgtccagttct
agacggggcgtgtactaccccgacgacatcttcagatccgacgtgctgcatctgacccag
gactacttcctgcctttcgacagcaacgtgacccggtacttcagcctgaacgccaacgga
cccgaccggatcgtgtacttcgacaaccctatcatccccttcaaggacggggtgtacttt
gccgccaccgagaagtccaacgtgatcagaggctggatcttcggcagcaccctggacaat
accagccagagcgtgatcatcgtgaacaacagcaccaacgtcatcatccgcgtgtgcaac
ttcgacctgtgcaacgacccattcttcaccgtgtccagaccaaccgacaagcacatcaag
acctggtccatccgcgagttcgccgtgtaccagagcgccttcaattgcaccttcgagtac
gtgtccaagagctttctgctggacgtggccgagaagcccggcaactttaagcacctgaga
gaattcgtgttcaagaacgtggacggcttcctgaacgtgtacagcacctacaagcccatc
aacgtggtgtccggcctgcctacaggattcagcgtgctgaagcccatcctgaagctgccc
ctgggcatcaacatcaccagcttcagagtgctgctgaccatgttcagaggcgaccctaca
cctggccacaccaccgctaattggctgacagccgccgctgcctactacgtgggatacctg
aagcctaccaccttcatgctcaagtacaacgagaacggcaccatcaccgacgccgtggac
tgtagccaaaatcctctggccgagctgaagtgcaccctgaagaacttcaacgtggacaag
ggcatctaccagaccagcaacttccgggtgtcccctacacaagaggtcgtgcggttcccc
aatatcaccaatctgtgccccttcgacaaggtgttcaacgccaccagatttcccagcgtg
tacgcctgggagcgcaccaagatttccgattgcgtggccgactacaccgtgctgtataac
tccacctccttcagcaccttcaagtgctacggcgtgtccccaagcaagctgatcgatctg
tgcttcacctctgtgtacgccgacaccttcctgatccggtgtagcgaagtgcgacaggtg
gcacctggacagacaggcgtgatcgccgattacaactacaagctgcccgacgacttcacc
ggctgtgtgatcgcctggaataccgccaagcaggatacaggcagcagcggcaactacaac
tactactacagaagccaccgcaagaccaagctgaagcctttcgagagggacctgagcagc
gacgagtgtagccctgatggcaagccttgtacacctcctgccttcaatggcgtgcggggc
ttcaactgctacttcaccctgagcacctacgacttcaaccccaacgtgcccgtggaatac
caggccacaagagtggtggtgctgagcttcgagctgctgaatgcccctgccacagtgtgt
ggccctaagctgtctacccagctggtcaagaaccagtgcgtgaacttcaatttcaacggc
ctgaaaggcaccggcgtgctgaccgccagcagcaagagattccagagcttccagcagttc
ggcagggacgccagcgatttcacagatagcgtcagagatccccagacactggaaatcctg
gacatcagcccttgcagcttcggcggagtgtctgtgatcacccctggcaccaatacctct
agcgaggtggcagtgctgtaccaggacgtgaactgcaccgatgtgcctacagccatccac
gccgatcagctgacaccagcttggagagtgtactctaccggtgtcaacgtgttccagaca
caagccggctgtctgattggagccgaacacgtgaacgccagctacgagtgcgacatccct
atcggagccggcatctgtgcctcttaccacaccgcctctaacagccccagaatcctgaga
agcaccggccagaaatccatcgtggcctacacaatgtctctgggcgccgagaactctatc
gcctacgccaacaactccattgctatccccaccaacttcagcatctccgtgaccaccgaa
gtgatgcctgtgtccatggccaagaccagcgtggactgcacaatgtacatctgcggcgac
agcctggaatgcagcaacctgctgctccagtacggcagcttctgcacccagctgaataga
gccctgaccggaatcgccatcgagcaggacaagaacacccaagaggtgttcgcccaagtg
aagcagatgtataagacccctgccatcaaggacttcggcggctttaacttcagccagatc
ctgcctgatcctagcaagcccaccaagcggagcttcatcgaggacctgctgttcaacaaa
gtgaccctggccgacgccggctttatgaagcagtatggcgagtgcctgggcgacatctct
gccagggatctgatttgcgcccagaagttcaacggactgaccgtgctgcctcctctgctg
accgatgagatgatcgccgcctatacagccgctctggtgtctggcacagctaccgccgga
tggacatttggagctggcgccgctctccagattccattcgctatgcagatggcctaccgc
ttcaacggcatcggcgtgacccagaacgtgctgtacgagaaccagaagcagatcgccaac
cagttcaacaaggccatcagtcagatccaagagagcctgaccacaaccagcacagccctg
ggaaagctccaggacgtcgtgaaccagaatgcccaggctctgaacaccctggtcaagcag
ctgagcagcaatttcggcgccatcagctccgtgctgaacgacatcctgagccggctggat
aaggtggaagccgaggtgcagatcgaccggctgattacaggcagactccagtctctccag
acctacgtgacacagcagctgatcagagccgccgagattagagcctctgccaatctggcc
gccaccaagatgtctgagtgtgtgctgggccagtctaagagagtggacttctgcggcaag
ggctaccacctgatgagcttccctcaggctgctcctcacggcgtggtgtttctgcacgtg
acatacgtgcccagccaagagcggaacttcacaactgccccagccatctgccacgagggc
aaagcctactttcccagagaaggcgtgttcgtgtccaacggcacctcctggttcatcacc
cagagaaacttctacagccctcagatcatcaccaccgacaacaccttcgtgtccggcaac
tgcgacgtggtcatcggcatcatcaacaataccgtgtacgaccctctccagccagaactg
gatagcttcaaagaggaactcgacaagtacttcaagaatcacacaagccccgacgtggac
ctgggcgatatcagcggaatcaatgccagcgtggtcaacatccagaaagagatcgacaga
ctgaacgaggtggccaagaacctgaacgagtccctgatcgacctgcaagagctggggaag
tacgagcagtacatcaagtggccttggtacgtgtggctgggctttatcgccggactgatc
gccattgtgatggccaccatcctgctgtgctgcatgacaagctgctgtagctgcctgaag
ggcgcctgtagctgtggcagctgctgcaagttcgacgaggacgattctgagcctgtgctg
aaaggcgtgaagctgcactacacc
>Wuhan_Node1_tr (COV_T2_4) (SEQ ID NO: 15)
Amino acid sequence:
MFLFLFIIIFAFFLLSAKANERCGIFTSKPQPKLAQVSSSRRGVYYPDDIFRSDVLHLTQDYELPFDS
NVTRYFSLNANGPDRIVYFDNPIIPFKDGVYFAATEKSNVIRGWIFGSTLDNTSQSVIIVNNSTNVII
RVCNFDLCNDPFFTVSRPTDKHIKTWSIREFAVYQSAFNCTFEYVSKSELLDVAEKPGNEKHLREFVF
KNVDGFLNVYSTYKPINVVSGLPTGFSVLKPILKLPLGINITSFRVLLTMERGDPTPGHTTANWLTAA
AAYYVGYLKPTTFMLKYNENGTITDAVDCSQNPLAELKCTLKNENVDKGIYQTSNERVSPTQEVVRFP
NITNLCPFDKVFNATRFPSVYAWERTKISDCVADYTVLYNSTSFSTFKCYGVSPSKLIDLCFTSVYAD
TFLIRCSEVRQVAPGQTGVIADYNYKLPDDETGCVIAWNTAKQDTGSSGNYNYYYRSHRKTKLKPFER
DLSSDECSPDGKPCTPPAFNGVRGENCYFTLSTYDENPNVPVEYQATRVVVLSFELLNAPATVCGPKL
STQLVKNQCVNFNENGLKGTGVLTASSKRFQSFQQFGRDASDETDSVRDPQTLEILDISPCSFGGVSV
ITPGTNTSSEVAVLYQDVNCTDVPTAIHADQLTPAWRVYSTGVNVFQTQAGCLIGAEHVNASYECDIP
IGAGICASYHTASNSPRILRSTGQKSIVAYTMSLGAENSIAYANNSIAIPTNFSISVTTEVMPVSMAK
TSVDCTMYICGDSLECSNLLLQYGSFCTQLNRALTGIAIEQDKNTQEVFAQVKQMYKTPAIKDEGGFN
FSQILPDPSKPTKRSFIEDLLENKVTLADAGEMKQYGECLGDISARDLICAQKENGLTVLPPLLTDEM
IAAYTAALVSGTATAGWTFGAGAALQIPFAMQMAYRENGIGVTQNVLYENQKQIANQFNKAISQIQES
LTTTSTALGKLQDVVNQNAQALNTLVKQLSSNFGAISSVLNDILSRLDKVEAEVQIDRLITGRLQSLQ
TYVTQQLIRAAEIRASANLAATKMSECVLGQSKRVDFCGKGYHLMSFPQAAPHGVVFLHVTYVPSQER
NFTTAPAICHEGKAYFPREGVFVSNGTSWFITQRNFYSPQIITTDNTFVSGNCDVVIGIINNTVYDPL
QPELDSFKEELDKYFKNHTSPDVDLGDIS
>Wuhan_Node1_tr (COV_T2_4) (SEQ ID NO: 16)
Nucleic acid sequence:
atgtttctgttcctcttcattattatcttcgcattcttcctgctgagcgccaaggccaac
gagagatgcggcatcttcaccagcaagccccagcctaagctggcccaggtgtccagttct
agacggggcgtgtactaccccgacgacatcttcagatccgacgtgctgcatctgacccag
gactacttcctgcctttcgacagcaacgtgacccggtacttcagcctgaacgccaacgga
cccgaccggatcgtgtacttcgacaaccctatcatccccttcaaggacggggtgtacttt
gccgccaccgagaagtccaacgtgatcagaggctggatcttcggcagcaccctggacaat
accagccagagcgtgatcatcgtgaacaacagcaccaacgtcatcatccgcgtgtgcaac
ttcgacctgtgcaacgacccattcttcaccgtgtccagaccaaccgacaagcacatcaag
acctggtccatccgcgagttcgccgtgtaccagagcgccttcaattgcaccttcgagtac
gtgtccaagagctttctgctggacgtggccgagaagcccggcaactttaagcacctgaga
gaattcgtgttcaagaacgtggacggcttcctgaacgtgtacagcacctacaagcccatc
aacgtggtgtccggcctgcctacaggattcagcgtgctgaagcccatcctgaagctgccc
ctgggcatcaacatcaccagcttcagagtgctgctgaccatgttcagaggcgaccctaca
cctggccacaccaccgctaattggctgacagccgccgctgcctactacgtgggatacctg
aagcctaccaccttcatgctcaagtacaacgagaacggcaccatcaccgacgccgtggac
tgtagccaaaatcctctggccgagctgaagtgcaccctgaagaacttcaacgtggacaag
ggcatctaccagaccagcaacttccgggtgtcccctacacaagaggtcgtgcggttcccc
aatatcaccaatctgtgccccttcgacaaggtgttcaacgccaccagatttcccagcgtg
tacgcctgggagcgcaccaagatttccgattgcgtggccgactacaccgtgctgtataac
tccacctccttcagcaccttcaagtgctacggcgtgtccccaagcaagctgatcgatctg
tgcttcacctctgtgtacgccgacaccttcctgatccggtgtagcgaagtgcgacaggtg
gcacctggacagacaggcgtgatcgccgattacaactacaagctgcccgacgacttcacc
ggctgtgtgatcgcctggaataccgccaagcaggatacaggcagcagcggcaactacaac
tactactacagaagccaccgcaagaccaagctgaagcctttcgagagggacctgagcagc
gacgagtgtagccctgatggcaagccttgtacacctcctgccttcaatggcgtgcggggc
ttcaactgctacttcaccctgagcacctacgacttcaaccccaacgtgcccgtggaatac
caggccacaagagtggtggtgctgagcttcgagctgctgaatgcccctgccacagtgtgt
ggccctaagctgtctacccagctggtcaagaaccagtgcgtgaacttcaatttcaacggc
ctgaaaggcaccggcgtgctgaccgccagcagcaagagattccagagcttccagcagttc
ggcagggacgccagcgatttcacagatagcgtcagagatccccagacactggaaatcctg
gacatcagcccttgcagcttcggcggagtgtctgtgatcacccctggcaccaatacctct
agcgaggtggcagtgctgtaccaggacgtgaactgcaccgatgtgcctacagccatccac
gccgatcagctgacaccagcttggagagtgtactctaccggtgtcaacgtgttccagaca
caagccggctgtctgattggagccgaacacgtgaacgccagctacgagtgcgacatccct
atcggagccggcatctgtgcctcttaccacaccgcctctaacagccccagaatcctgaga
agcaccggccagaaatccatcgtggcctacacaatgtctctgggcgccgagaactctatc
gcctacgccaacaactccattgctatccccaccaacttcagcatctccgtgaccaccgaa
gtgatgcctgtgtccatggccaagaccagcgtggactgcacaatgtacatctgcggcgac
agcctggaatgcagcaacctgctgctccagtacggcagcttctgcacccagctgaataga
gccctgaccggaatcgccatcgagcaggacaagaacacccaagaggtgttcgcccaagtg
aagcagatgtataagacccctgccatcaaggacttcggcggctttaacttcagccagatc
ctgcctgatcctagcaagcccaccaagcggagcttcatcgaggacctgctgttcaacaaa
gtgaccctggccgacgccggctttatgaagcagtatggcgagtgcctgggcgacatctct
gccagggatctgatttgcgcccagaagttcaacggactgaccgtgctgcctcctctgctg
accgatgagatgatcgccgcctatacagccgctctggtgtctggcacagctaccgccgga
tggacatttggagctggcgccgctctccagattccattcgctatgcagatggcctaccgc
ttcaacggcatcggcgtgacccagaacgtgctgtacgagaaccagaagcagatcgccaac
cagttcaacaaggccatcagtcagatccaagagagcctgaccacaaccagcacagccctg
ggaaagctccaggacgtcgtgaaccagaatgcccaggctctgaacaccctggtcaagcag
ctgagcagcaatttcggcgccatcagctccgtgctgaacgacatcctgagccggctggat
aaggtggaagccgaggtgcagatcgaccggctgattacaggcagactccagtctctccag
acctacgtgacacagcagctgatcagagccgccgagattagagcctctgccaatctggcc
gccaccaagatgtctgagtgtgtgctgggccagtctaagagagtggacttctgcggcaag
ggctaccacctgatgagcttccctcaggctgctcctcacggcgtggtgtttctgcacgtg
acatacgtgcccagccaagagcggaacttcacaactgccccagccatctgccacgagggc
aaagcctactttcccagagaaggcgtgttcgtgtccaacggcacctcctggttcatcacc
cagagaaacttctacagccctcagatcatcaccaccgacaacaccttcgtgtccggcaac
tgcgacgtggtcatcggcatcatcaacaataccgtgtacgaccctctccagccagaactg
gatagcttcaaagaggaactcgacaagtacttcaagaatcacacaagccccgacgtggac
ctgggcgatatcagt
>Wuhan_Node1_RBD (CoV_T2_7) (SEQ ID NO: 17)
Amino acid sequence:
RVSPTQEVVRFPNITNLCPFDKVENATRFPSVYAWERTKISDCVADYTVLYNSTSFSTEKCYGVSPSK
LIDLCFTSVYADTFLIRCSEVRQVAPGQTGVIADYNYKLPDDFTGCVIAWNTAKQDTGSSGNYNYYYR
SHRKTKLKPFERDLSSDECSPDGKPCTPPAFNGVRGENCYFTLSTYDENPNVPVEYQATRVVVLSFEL
LNAPATVCGPKLSTQ
>Wuhan_Node1_RBD (CoV_T2_7) (SEQ ID NO: 18)
Nucleic acid sequence:
cgggtgtcccctacacaagaggtcgtgcggttccccaatatcaccaatctgtgccccttc
gacaaggtgttcaacgccaccagatttcccagcgtgtacgcctgggagcgcaccaagatt
tccgattgcgtggccgactacaccgtgctgtataactccacctccttcagcaccttcaag
tgctacggcgtgtccccaagcaagctgatcgatctgtgcttcacctctgtgtacgccgac
accttcctgatccggtgtagcgaagtgcgacaggtggcacctggacagacaggcgtgatc
gccgattacaactacaagctgcccgacgacttcaccggctgtgtgatcgcctggaatacc
gccaagcaggatacaggcagcagcggcaactacaactactactacagaagccaccgcaag
accaagctgaagcctttcgagagggacctgagcagcgacgagtgtagccctgatggcaag
ccttgtacacctcctgccttcaatggcgtgcggggcttcaactgctacttcaccctgagc
acctacgacttcaaccccaacgtgcccgtggaataccaggccacaagagtggtggtgctg
agcttcgagctgctgaatgcccctgccacagtgtgtggccctaagctgtctacccag
Wuhan_Node1 (CoV_T2_1) (full length S protein amino acid sequence,
with RBD residues shown in bold, and residues not present in
truncated S protein shown underlined) (SEQ ID NO: 13)

| | |
|---|---|
| MFLFLFIIIF AFFLLSAKAN ERCGIFTSKP QPKLAQVSSS RRGVYYPDDI FRSDVLHLTQ | 60 |
| DYFLPFDSNV TRYFSLNANG PDRIVYFDNP IIPFKDGVYF AATEKSNVIR GWIFGSTLDN | 120 |
| TSQSVIIVNN STNVIIRVCN FDLCNDPFFT VSRPTDKHIK TWSIREFAVY QSAFNCTFEY | 180 |
| VSKSFLLDVA EKPGNFKHLR EFVFKNVDGF LNVYSTYKPI NVVSGLPTGF SVLKPILKLP | 240 |
| LGINITSFRV LLTMFRGDPT PGHTTANWLT AAAAYYVGYL KPTTFMLKYN ENGTITDAVD | 300 |
| CSQNPLAELK CTLKNFNVDK GIYQTSNFRV SPTQEVVRFP NITNLCPFDK VFNATRFPSV | 360 |
| YAWERTKISD CVADYTVLYN STSFSTFKCY GVSPSKLIDL CFTSVYADTF LIRCSEVRQV | 420 |
| APGQTGVIAD YNYKLPDDFT GCVIAWNTAK QDTGSSGNYN YYYRSHRKTK LKPFERDLSS | 480 |
| DECSPDGKPC TPPAFNGVRG FNCYFTLSTY DFNPNVPVEY QATRVVVLSF ELLNAPATVC | 540 |
| GPKLSTQLVK NQCVNFNFNG LKGTGVLTAS SKRFQSFQQF GRDASDFTDS VRDPQTLEIL | 600 |
| DISPCSFGGV SVITPGTNTS SEVAVLYQDV NCTDVPTAIH ADQLTPAWRV YSTGVNVFQT | 660 |
| QAGCLIGAEH VNASYECDIP IGAGICASYH TASNSPRILR STGQKSIVAY TMSLGAENSI | 720 |
| AYANNSIAIP TNFSISVTTE VMPVSMAKTS VDCTMYICGD SLECSNLLLQ YGSFCTQLNR | 780 |
| ALTGIAIEQD KNTQEVFAQV KQMYKTPAIK DFGGFNFSQI LPDPSKPTKR SFIEDLLFNK | 840 |
| VTLADAGFMK QYGECLGDIS ARDLICAQKF NGLTVLPPLL TDEMIAAYTA ALVSGTATAG | 900 |
| WTFGAGAALQ IPFAMQMAYR FNGIGVTQNV LYENQKQIAN QFNKAISQIQ ESLTTTSTAL | 960 |
| GKLQDVVNQN AQALNTLVKQ LSSNFGAISS VINDILSRLD KVEAEVQIDR LITGRLQSLQ | 1020 |
| TYVTQQLIRA AEIRASANLA ATKMSECVLG QSKRVDFCGK GYHLMSFPQA APHGVVFLHV | 1080 |
| TYVPSQERNF TTAPAICHEG KAYFPREGVF VSNGTSWFIT QRNFYSPQII TTDNTFVSGN | 1140 |
| CDVVIGIINN TVYDPLQPEL DSFKEELDKY FKNHTSPDVD LGDISGINAS VVNIQKEIDR | 1200 |
| LNEVAKNLNE SLIDLQELGK YEQYIKWPWY VWLGFIAGLI AIVMATILLC CMTSCCSCLK | 1260 |
| GACSCGSCCK FDEDDSEPVL KGVKLHYT | 1288 |

### EXAMPLE 2

Alignment of full-length S-protein amino acid sequence of CoV_T2_1 (Wuhan_Node1) with
AY274119

Score = 55060.0
Length of alignment = 1284
Sequence Wuhan_Node1/5-1288 (Sequence length = 1288) (SEQ ID NO: 13)
Sequence AY274119/1-1255 (Sequence length = 1255) (SEQ ID NO: 1)

| | |
|---|---|
| Wuhan_Node1/5-1288 | LFIIIFAFFLLSAKANERCGIFTSKPQPKLAQVSSSRRGVYYPDDIFRSDVLH |
| | .\|\|... . \| \|. .\|\| \| \|. .\| .\|\| \|\|\|\|\|\|\|.\|\|\|\|\| \| |
| AY274119/1-1255 | MFIFLLELTLTSGSDLDRCTTFDDVQAPNYTQHTSSMRGVYYPDEIFRSDTLY |
| Wuhan_Node1/5-1288 | LTQDYFLPFDSNVTRYFSLNANGPDRIVYFDNPIIPFKDGVYFAATEKSNVIR |
| | \|\|\|\| \|\|\|\| \|\|\|\| . ..\| . \|.\|\|.\|\|\|\|\|\|.\|\|\|\|\|\|\|\|\|\|.\| |
| AY274119/1-1255 | LTQDLFLPFYSNVTGFHTIN-----HT--FGNPVIPFKDGIYFAATEKSNVVR |
| Wuhan_Node1/5-1288 | GWIFGSTLDNTSQSVIIVNNSTNVIIRVCNFDLCNDPFFTVSRPTDKHIKTWS |
| | \|\|.\|\|\|\|..\| \|\|\|\|\|\|.\|\|\|\|\|\|.\|\| \|\|\|.\|\|..\|\|\|.\|\|.\| . .\| . |
| AY274119/1-1255 | GWVFGSTMNNKSQSVIIINNSTNVVIRACNFELCDNPFFAVSKPMG--TQTHT |
| Wuhan_Node1/5-1288 | IREFAVYQSAFNCTFEYVSKSFLLDVAEKPGNFKHLREFVFKNVDGFLNVYST |
| | ....\|\|\|\|\|\|\|\|.\| .\| \|\|\|.\|\|.\|\|\|\|\|\|\|\|\|\|\|\|\| \|\|\|\| \|\| |
| AY274119/1-1255 | ----MIFDNAFNCTFEYISDAFSLDVSEKSGNFKHLREFVEKNKDGFLYVYKG |
| Wuhan_Node1/5-1288 | YKPINVVSGLPTGFSVLKPILKLPLGINITSFRVLLTMFRGDPTPGHTTANWL |
| | \|.\|\|.\|\| .\|\|.\|\|. \|\|\|\|.\|\|\|\|\|\|\|\|\|.\|\| .\|\| \| .\|.. \| |
| AY274119/1-1255 | YQPIDVVRDLPSGFNTLKPIFKLPLGINITNFRAILTAF----SPAQDI--WG |
| Wuhan_Node1/5-1288 | TAAAAYYVGYLKPTTFMLKYNENGTITDAVDCSQNPLAELKCTLKNFNVDKGI |
| | \|.\|\|\|\|.\|\|\|\|\|\|\|\|\|\|\|\|\|.\|\|\|\|\|\|\|\|\|\|\|\|\|\|\|\|\|\|\|\|\|..\|.\|..\|\|\|\| |
| AY274119/1-1255 | TSAAAYFVGYLKPTTFMLKYDENGTITDAVDCSQNPLAELKCSVKSFEIDKGI |
| Wuhan_Node1/5-1288 | YQTSNFRVSPTQEVVRFPNITNLCPFDKVFNATRFPSVYAWERTKISDCVADY |
| | \|\|\|\|\|\|\|\| \|. .\|\|\|\|\|\|\|\|\|\|\|\|\|. \|\|\|\|\|.\|\|\|\|\|\|\|\|\| \|\|\|.\|\|\|\|\| |
| AY274119/1-1255 | YQTSNFRVVPSGDVVRFPNITNLCPFGEVFNATKEPSVYAWERKKISNCVADY |
| Wuhan_Node1/5-1288 | TVLYNSTSFSTFKCYGVSPSKLIDLCFTSVYADTFLIRCSEVRQVAPGQTGVI |
| | .\|\|\|\|\|\| \|\|\|\|\|\|\|\|\|\|..\|\| \|\|\|\|..\|\|\|\|.\|... .\|\|\|.\|\|\|\|\|\|\|\| |
| AY274119/1-1255 | SVLYNSTFFSTFKCYGVSATKLNDLCFSNVYADSFVVKGDDVRQIAPGQTGVI |
| Wuhan_Node1/5-1288 | ADYNYKLPDDFTGCVIAWNTAKQDTGSSGNYNYYYRSHRKTKLKPFERDLSSD |
| | \|\|\|\|\|\|\|\|\|\|\| \|\|\|.\|\|\|\| . \|. \|.\|\|\|\|\| \|\| \| \|\|.\|\|\|\|\|.\|. |
| AY274119/1-1255 | ADYNYKLPDDFMGCVLAWNTRNIDATSTGNYNYKYRYLRHGKLRPFERDISNV |
| Wuhan_Node1/5-1288 | ECSPDGKPCTPPAFNGVRGFNCYFTLSTYDFNPNVPVEYQATRVVVLSFELLN |
| | \|\|\|\|\|\|\|\|\|\|\| .\|\|\| \|. \|.\| . \|\|. \|\|\|\|\|\|\|\|\|\|\| |
| AY274119/1-1255 | PFSPDGKPCTPPA------LNCYWPLNDYGFYTTTGIGYQPYRVVVLSFELLN |
| Wuhan_Node1/5-1288 | APATVCGPKLSTQLVKNQCVNFNFNGLKGTGVLTASSKRFQSFQQFGRDASDF |
| | \|\|\|\|\|\|\|\|\|\|\|\|.\|.\|\|\|\|\|\|\|\|\|\|\|\| \|\|\|\|\|\|.\|\|\|\|\|\|.\|\|\|\|\|\|\| \|\|\| |
| AY274119/1-1255 | APATVCGPKLSTDLIKNQCVNFNFNGLTGTGVLTPSSKRFQPFQQFGRDVSDF |
| Wuhan_Node1/5-1288 | TDSVRDPQTLEILDISPCSFGGVSVITPGTNTSSEVAVLYQDVNCTDVPTAIH |
| | \|\|\|\|\|\|\|.\| \|\|\|\|\|\|\|\|.\|\|\|\|\|\|\|\|\|\|\|\|.\|\|\|\|\|\|\|\|\|\|\|\|\|\|\|\|.\|\|\|\| |
| AY274119/1-1255 | TDSVRDPKTSEILDISPCAFGGVSVITPGTNASSEVAVLYQDVNCTDVSTAIH |
| Wuhan_Node1/5-1288 | ADQLTPAWRVYSTGVNVFQTQAGCLIGAEHVNASYECDIPIGAGICASYHTAS |
| | \|\|\|\|\|\|\|\|\|.\|\|\|\| \|\|\|\|\|\|\|\|\|\|\|\|\|\|\|\|..\|\|\|\|\|\|\|\|\|\|\|\|\|\|\|\|\|\| \| |
| AY274119/1-1255 | ADQLTPAWRIYSTGNNVFQTQAGCLIGAEHVDTSYECDIPIGAGICASYHTVS |
| Wuhan_Node1/5-1288 | NSPRILRSTGQKSIVAYTMSLGAENSIAYANNSIAIPTNFSISVTTEVMPVSM |
| | .\|\|\|\|.\|\|\|\|\|\|\|\|\|\|\|\|\|..\|\|\|\|.\|\|.\|\|\|\|\|\|\|\|\|\|.\|\|\|\|\|\|\|\|\| |
| AY274119/1-1255 | ----LLRSTSQKSIVAYTMSLGADSSIAYSNNTIAIPTNFSISITTEVMPVSM |
| Wuhan_Node1/5-1288 | AKTSVDCTMYICGDSLECSNLLLQYGSFCTQLNRALTGIAIEQDKNTQEVFAQ |
| | \|\|\|\|\|\|\| \|\|\|\|\|\|\| \|\|.\|\|\|\|\|\|\|\|\|\|\|\|\|\|\|\|\|.\|\|\| \|\|\|.\|\|.\|\|\|\|\| |
| AY274119/1-1255 | AKTSVDCNMYICGDSTECANLLLQYGSFCTQLNRALSGIAAEQDRNTREVFAQ |
| Wuhan_Node1/5-1288 | VKQMYKTPAIKDFGGFNFSQILPDPSKPTKRSFIEDLLFNKVTLADAGFMKQY |
| | \|\|\|\|\|\|\|\|..\| \|\|\|\|\|\|\|\|\|\|\|\|\| \|\|\|\|\|\|\|\|\|\|\|\|\|\|\|\|\|\|\|\|\|\|\|\|\|\|\| |
| AY274119/1-1255 | VKQMYKTPTLKYFGGFNFSQILPDPLKPTKRSFIEDLLFNKVTLADAGFMKQY |
| Wuhan_Node1/5-1288 | GECLGDISARDLICAQKFNGLTVLPPLLTDEMIAAYTAALVSGTATAGWTFGA |
| | \|\|\|\|\|\|\|.\|\|\|\|\|\|\|\|\|\|\|\|\|\|\|\|\|\|\|\|\|\|.\|\|\|\|\|\|\|\|\|\|\|\|\|\|\|\|\|\|\|\|\|\| |
| AY274119/1-1255 | GECLGDINARDLICAQKFNGLTVLPPLLTDDMIAAYTAALVSGTATAGWTFGA |
| Wuhan_Node1/5-1288 | GAALQIPFAMQMAYRFNGIGVTQNVLYENQKQIANQFNKAISQIQESLTTTST |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119/1-1255 | GAALQIPFAMQMAYRFNGIGVTQNVLYENQKQIANQFNKAISQIQESLTTTST |
| Wuhan_Node1/5-1288 | ALGKLQDVVNQNAQALNTLVKQLSSNFGAISSVLNDILSRLDKVEAEVQIDRL |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119/1-1255 | ALGKLQDVVNQNAQALNTLVKQLSSNFGAISSVLNDILSRLDKVEAEVQIDRL |
| Wuhan_Node1/5-1288 | ITGRLQSLQTYVTQQLIRAAEIRASANLAATKMSECVLGQSKRVDFCGKGYHL |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119/1-1255 | ITGRLQSLQTYVTQQLIRAAEIRASANLAATKMSECVLGQSKRVDFCGKGYHL |
| Wuhan_Node1/5-1288 | MSFPQAAPHGVVFLHVTYVPSQERNFTTAPAICHEGKAYFPREGVFVSNGTSW |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\| |
| AY274119/1-1255 | MSFPQAAPHGVVFLHVTYVPSQERNFTTAPAICHEGKAYFPREGVFVENGTSW |
| Wuhan_Node1/5-1288 | FITQRNFYSPQIITTDNTFVSGNCDVVIGIINNTVYDPLQPELDSFKEELDKY |
| | \|\|\|\|\|\|\|.\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119/1-1255 | FITQRNFFSPQIITTDNTFVSGNCDVVIGIINNTVYDPLQPELDSFKEELDKY |
| Wuhan_Node1/5-1288 | FKNHTSPDVDLGDISGINASVVNIQKEIDRLNEVAKNLNESLIDLQELGKYEQ |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119/1-1255 | FKNHTSPDVDLGDISGINASVVNIQKEIDRLNEVAKNLNESLIDLQELGKYEQ |
| Wuhan_Node1/5-1288 | YIKWPWYVWLGFIAGLIAIVMATILLCCMTSCCSCLKGACSCGSCCKFDEDDS |
| | \|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\| |
| AY274119/1-1255 | YIKWPWYVWLGFIAGLIAIVMVTILLCCMTSCCSCLKGACSCGSCCKFDEDDS |
| Wuhan_Node1/5-1288 | EPVLKGVKLHYT |
| | \|\|\|\|\|\|\|\|\|\|\|\| |
| AY274119/1-1255 | EPVLKGVKLHYT |

Percentage ID = 82.32

### EXAMPLE 3

Alignment of full-length S-protein amino acid sequence of CoV_T2_1 (Wuhan_Node1) with
EPI_ISL_402119

Score = 53960.0
Length of alignment = 1280
Sequence Wuhan_Node1/9-1288 (Sequence length = 1288) (SEQ ID NO: 13)
Sequence EPI_ISL_402119/1-1273 (Sequence length = 1273) (SEQ ID NO: 7)

| | |
|---|---|
| Wuhan_Node1/9-1288 | IFAFFLLSAKANERCGIFTSKPQPKLAQVSSSRRGVYYPDDIFRSDVLHL |
| | .\| .\|..\| . . .\| .\|.. \| \| .\| \|\|\|\|\|\|\| .\|\|\| \|\|\| |
| EPI_ISL_402119/1-1273 | MFVFLVLLPLVSSQCVNLTTRTQLPPAYTNSFTRGVYYPDKVFRSSVLHS |
| Wuhan_Node1/9-1288 | TQDYFLPFDSNVTRYFSLNANGPDRIVYFDNPIIPFKDGVYFAATEKSNV |
| | \|\|\| \|\|\|\| \|\|\|\|.. ... .\| . \|\|\|\|..\|\|.\|\|\|\|\|\|.\|\|\|\|\|. |
| EPI_ISL_402119/1-1273 | TQDLFLPFFSNVTWFHAIHVSGTNGTKRFDNPVLPFNDGVYFASTEKSNI |
| Wuhan_Node1/9-1288 | IRGWIFGSTLDNTSQSVIIVNNSTNVIIRVCNFDLCNDPFFTVSRPTDKH |
| | \|\|\|\|\|\|\|.\|\|\|. .\|\|..\|\|\|\|.\|\|\|.\|.\|\|.\|..\|\|\|\|\|. \| .\|. |
| EPI_ISL_402119/1-1273 | IRGWIFGTTLDSKTQSLLIVNNATNVVIKVCEFQFCNDPFLGVY--YHKN |
| Wuhan_Node1/9-1288 | IKTWSIREFAVYQSAFNCTFEYVSKSFLLDVAEKPGNFKHLREFVFKNVD |
| | \|.\| \|\| \|\| \|\| \|\|\|\|\|\|\|\|..\|\|.\|. \| \|\|\|\|.\|\|\|\|\|\|\|\|.\| |
| EPI_ISL_402119/1-1273 | NKSWMESEFRVYSSANNCTFEYVSQPFLMDLEGKQGNFKNLREFVFKNID |
| Wuhan_Node1/9-1288 | GFLNVYSTYKPINVVSGLPTGFSVLKPILKLPLGINITSFRVLLTMFRGD |
| | \|....\|\| \|\|\|.\| .\|\| \|\|\| \| \|.. \|\|.\|\|\|\|\| \|. \|\|.. \|. |
| EPI_ISL_402119/1-1273 | GYFKIYSKHTPINLVRDLPQGFSALEPLVDLPIGINITRFQTLLALHRSY |
| Wuhan_Node1/9-1288 | PTPGHTTANWLTAAAAYYVGYLKPTTFMLKYNENGTITDAVDCSQNPLAE |
| | \|\|\|.... \| ..\|\|\|\|\|\|\|\|\|.\| \|\|.\|\|\|\|\|\|\|\|\|\|\|\|\|\|\|. .\|\|.\| |
| EPI_ISL_402119/1-1273 | LTPGDSSSGWTAGAAAYYVGYLQPRTFLLKYNENGTITDAVDCALDPLSE |
| Wuhan_Node1/9-1288 | LKCTLKNFNVDKGIYQTSNFRVSPTQEVVRFPNITNLCPFDKVFNATRFP |
| | \|\|\|\|\|.\| \|.\|\|\|\|\|\|\|\|\|\|\| \|\|. .\|\|\|\|\|\|\|\|\|\|\|\|. \|\|\|\|\|\|\|. |
| EPI_ISL_402119/1-1273 | TKCTLKSFTVEKGIYQTSNFRVQPTESIVRFPNITNLCPFGEVFNATRFA |
| Wuhan_Node1/9-1288 | SVYAWERTKISDCVADYTVLYNSTSFSTFKCYGVSPSKLIDLCFTSVYAD |
| | \|\|\|\|\|.\| .\|\|.\|\|\|\|\|.\|\|\|\|\|.\|\|\|\|\|\|\|\|\|\|\|\|.\|\| \|\|\|\|\|.\|\|\|\| |
| EPI_ISL_402119/1-1273 | SVYAWNRKRISNCVADYSVLYNSASFSTFKCYGVSPTKLNDLCFTNVYAD |
| Wuhan_Node1/9-1288 | TFLIRCSEVRQVAPGQTGVIADYNYKLPDDFTGCVIAWNTAKQDTGSSGN |
| | .\|.\|\| \|\|\|\|.\|\|\|\|\| \|\|\|\|\|\|\|\|\|\|\|\|\|\|\|\|\|\|\|\|\|. . \|. .\|\| |
| EPI_ISL_402119/1-1273 | SFVIRGDEVRQIAPGQTGKIADYNYKLPDDFTGCVIAWNSNNLDSKVGGN |
| Wuhan_Node1/9-1288 | YNYYYRSHRKTKLKPFERDLSSDECSPDGKPCTPPAFNGVRGFNCYFTLS |
| | \|\|\| \|\| \|\|..\|\|\|\|\|\|\|.\|.. ... \|\| \|\|\| \|\|\|\|\|\| \| |
| EPI_ISL_402119/1-1273 | YNYLYRLFRKSNLKPFERDISTEIYQAGSTPC-----NGVEGFNCYFPLQ |
| Wuhan_Node1/9-1288 | TYDFNPNVPVEYQATRVVVLSFELLNAPATVCGPKLSTQLVKNQCVNFNF |
| | .\|.\|.\| \| \|\|. \|\|\|\|\|\|\|\|\|\|.\|\|\|\|\|\|\|\|\| \|\|.\|\|\|\|.\|\|\|\|\|\| |
| EPI_ISL_402119/1-1273 | SYGFQPTNGVGYQPYRVVVLSFELLHAPATVCGPKKSTNLVKNKCVNFNF |
| Wuhan_Node1/9-1288 | NGLKGTGVLTASSKRFQSFQQFGRDASDFTDSVRDPQTLEILDISPCSFG |
| | \|\|\| \|\|\|\|\|\| \|.\|.\| .\|\|\|\|\|\|\| .\| \|\|.\|\|\|\|\|\|\|\|\|\|\|\|.\|\|\|\|\| |
| EPI_ISL_402119/1-1273 | NGLTGTGVLTESNKKFLPFQQFGRDIADTTDAVRDPQTLEILDITPCSFG |
| Wuhan_Node1/9-1288 | GVSVITPGTNTSSEVAVLYQDVNCTDVPTAIHADQLTPAWRVYSTGVNVF |
| | \|\|\|\|\|\|\|\|\|\|\|\|..\|\|\|\|\|\|\|\|\|\|\|.\|\| \|\|\|\|\|\|\|\|\|.\|\|\|\|\|\|\| \|\|\| |
| EPI_ISL_402119/1-1273 | GVSVITPGTNTSNQVAVLYQDVNCTEVPVAIHADQLTPTWRVYSTGSNVF |
| Wuhan_Node1/9-1288 | QTQAGCLIGAEHVNASYECDIPIGAGICASYHTASNSPRILRSTGQKSIV |
| | \|\|.\|\|\|\|\|\|\|\|\|\|\| \|\|\|\|\|\|\|\|\|\|\|\|\|\|\|\|.\| .\|\|\|\| \|\| . .\|\|. |
| EPI_ISL_402119/1-1273 | QTRAGCLIGAEHVNNSYECDIPIGAGICASYQTQTNSPRRARSVASQSII |
| Wuhan_Node1/9-1288 | AYTMSLGAENSIAYANNSIAIPTNFSISVTTEVMPVSMAKTSVDCTMYIC |
| | \|\|\|\|\|\|\|\|\|\|\|.\|\|.\|\|\|\|\|\|\|\|\|\|.\|\|\|\|\|\|..\|\|\|\|.\|\|\|\|\|\|\|\|\|\|\| |
| EPI_ISL_402119/1-1273 | AYTMSLGAENSVAYSNNSIAIPTNFTISVTTEILPVSMTKTSVDCTMYIC |
| Wuhan_Node1/9-1288 | GDSLECSNLLLQYGSFCTQLNRALTGIAIEQDKNTQEVFAQVKQMYKTPA |
| | \|\|\| \|\|\|\|\|\|\|\|\|\|\|\|\|\|\|\|\|\|\|\|\|\|\|\|.\|\|\|\|\|\|\|\|\|\|\|\|\|\|\|.\|\|\|\|. |
| EPI_ISL_402119/1-1273 | GDSTECSNLLLQYGSFCTQLNRALTGIAVEQDKNTQEVFAQVKQIYKTPP |
| Wuhan_Node1/9-1288 | IKDFGGFNFSQILPDPSKPTKRSFIEDLLFNKVTLADAGFMKQYGECLGD |
| | \|\|\|\|\|\|\|\|\|\|\|\|\|\|\|\|\|\|\|.\|\|\|\|\|\|\|\|\|\|\|\|\|\|\|\|\|\|\|\|.\|\|\|\|.\|\|\|\| |
| EPI_ISL_402119/1-1273 | IKDFGGFNFSQILPDPSKPSKRSFIEDLLFNKVTLADAGFIKQYGDCLGD |
| Wuhan_Node1/9-1288 | ISARDLICAQKFNGLTVLPPLLTDEMIAAYTAALVSGTATAGWTFGAGAA |
| | \|.\|\|\|\|\|\|\|\|\|\|\|\|\|\|\|\|\|\|\|\|\|\|\|\|\|\| \|\|.\|\|..\|\| \|.\|\|\|\|\|\|\|\|\| |
| EPI_ISL_402119/1-1273 | IAARDLICAQKFNGLTVLPPLLTDEMIAQYTSALLAGTITSGWTFGAGAA |
| Wuhan_Node1/9-1288 | LQIPFAMQMAYRFNGIGVTQNVLYENQKQIANQFNKAISQIQESLTTTST |
| | \|\|\|\|\|\|\|\|\|\|\|\|\|\|\|\|\|\|\|\|\|\|\|\|\|\|\|\| \|\|\|\|\|\| \|\|..\|\|.\|\|..\|.. |
| EPI_ISL_402119/1-1273 | LQIPFAMQMAYRFNGIGVTQNVLYENQKLIANQFNSAIGKIQDSLSSTAS |
| Wuhan_Node1/9-1288 | ALGKLQDVVNQNAQALNTLVKQLSSNFGAISSVLNDILSRLDKVEAEVQI |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| EPI_ISL_402119/1-1273 | ALGKLQDVVNQNAQALNTLVKQLSSNFGAISSVLNDILSRLDKVEAEVQI |
| Wuhan_Node1/9-1288 | DRLITGRLQSLQTYVTQQLIRAAEIRASANLAATKMSECVLGQSKRVDFC |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| EPI_ISL_402119/1-1273 | DRLITGRLQSLQTYVTQQLIRAAEIRASANLAATKMSECVLGQSKRVDFC |
| Wuhan_Node1/9-1288 | GKGYHLMSFPQAAPHGVVFLHVTYVPSQERNFTTAPAICHEGKAYFPREG |
| | \|\|\|\|\|\|\|\|\|\|\|.\|\|\|\|\|\|\|\|\|\|\|\|\|\|.\|\|.\|\|\|\|\|\|\|\|\|\|.\|\|\| \|\|\|\|\| |
| EPI_ISL_402119/1-1273 | GKGYHLMSFPQSAPHGVVFLHVTYVPAQEKNFTTAPAICHDGKAHFPREG |
| Wuhan_Node1/9-1288 | VFVSNGTSWFITQRNFYSPQIITTDNTFVSGNCDVVIGIINNTVYDPLQP |
| | \|\|\|\|\|\|\| \|\|.\|\|\|\|\|\| \|\|\|\|\|\|\|\|\|\|\|\|\|\|\|\|\|\|\|\|\|.\|\|\|\|\|\|\|\|\|\| |
| EPI_ISL_402119/1-1273 | VFVSNGTHWFVTQRNFYEPQIITTDNTFVSGNCDVVIGIVNNTVYDPLQP |
| Wuhan_Node1/9-1288 | ELDSFKEELDKYFKNHTSPDVDLGDISGINASVVNIQKEIDRLNEVAKNL |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| EPI_ISL_402119/1-1273 | ELDSFKEELDKYFKNHTSPDVDLGDISGINASVVNIQKEIDRLNEVAKNL |
| Wuhan_Node1/9-1288 | NESLIDLQELGKYEQYIKWPWYVWLGFIAGLIAIVMATILLCCMTSCCSC |
| | \|\|\|\|\|\|\|\|\|\|\|\|\|\|\|\|\|\|\|\|\|\|.\|\|\|\|\|\|\|\|\|\|\|\|\| \|\|.\|\|\|\|\|\|\|\|\|\| |
| EPI_ISL_402119/1-1273 | NESLIDLQELGKYEQYIKWPWYIWLGFIAGLIAIVMVTIMLCCMTSCCSC |
| Wuhan_Node1/9-1288 | LKGACSCGSCCKFDEDDSEPVLKGVKLHYT |
| | \|\|\| \|\|\|\|\|\|\|\|\|\|\|\|\|\|\|\|\|\|\|\|\|\|\|\|\|\| |
| EPI_ISL_402119/1-1273 | LKGCCSCGSCCKFDEDDSEPVLKGVKLHYT |

Percentage ID = 78.98

### EXAMPLE 4

Alignment of truncated S-protein amino acid sequence of CoV_T2_4 (Wuhan_Node1_tr)
with AY274119

Score = 49480.0
Length of alignment = 1181
Sequence Wuhan_Node1_tr/5-1185 (Sequence length = 1185) (SEQ ID NO: 15)
Sequence AY274119_tr(CoV_T2_2)/1-1152 (Sequence length = 1152) (SEQ ID NO: 3)

| | |
|---|---|
| Wuhan_Node1_tr/5-1185 | LFIIIFAFFLLSAKANERCGIFTSKPQPKLAQVSSSRRGVYYP |
| | .\|\|... . \| \|. .\|\| \| \|. .\| .\|\| \|\|\|\|\|\| |
| AY274119_tr(CoV_T2_2)/1-1152 | MFIFLLFLTLTSGSDLDRCTTFDDVQAPNYTQHTSSMRGVYYP |
| Wuhan_Node1_tr/5-1185 | DDIFRSDVLHLTQDYFLPFDSNVTRYFSLNANGPDRIVYFDNP |
| | \|.\|\|\|\|\| \| \|\|\|\| \|\|\|\| \|\|\|\| . ..\| . \|.\|\| |
| AY274119_tr(CoV_T2_2)/1-1152 | DEIFRSDTLYLTQDLFLPFYSNVTGFHTIN-----HT--FGNP |
| Wuhan_Node1_tr/5-1185 | IIPFKDGVYFAATEKSNVIRGWIFGSTLDNTSQSVIIVNNSTN |
| | .\|\|\|\|\|\|.\|\|\|\|\|\|\|\|\|\|.\|\|\|.\|\|\|\|..\| \|\|\|\|\|\|.\|\|\|\|\| |
| AY274119_tr(CoV_T2_2)/1-1152 | VIPFKDGIYFAATEKSNVVRGWVFGSTMNNKSQSVIIINNSTN |
| Wuhan_Node1_tr/5-1185 | VIIRVCNFDLCNDPFFTVSRPTDKHIKTWSIREFAVYQSAFNC |
| | \|.\|\| \|\|\|.\|\|..\|\|\|.\|\|.\| . .\| . ....\|\|\|\| |
| AY274119_tr(CoV_T2_2)/1-1152 | VVIRACNFELCDNPFFAVSKPMG--TQTHT----MIFDNAFNC |
| Wuhan_Node1_tr/5-1185 | TFEYVSKSFLLDVAEKPGNFKHLREFVFKNVDGFLNVYSTYKP |
| | \|\|\|\|.\| .\| \|\|\|.\|\|.\|\|\|\|\|\|\|\|\|\|\|\|\| \|\|\|\| \|\| \|.\| |
| AY274119_tr(CoV_T2_2)/1-1152 | TFEYISDAFSLDVSEKSGNFKHLREFVFKNKDGFLYVYKGYQP |
| Wuhan_Node1_tr/5-1185 | INVVSGLPTGFSVLKPILKLPLGINITSFRVLLTMFRGDPTPG |
| | \|.\|\| .\|\|.\|\|. \|\|\|\|.\|\|\|\|\|\|\|\|\|.\|\| .\|\| \| .\|. |
| AY274119_tr(CoV_T2_2)/1-1152 | IDVVRDLPSGFNTLKPIFKLPLGINITNFRAILTAF----SPA |
| Wuhan_Node1_tr/5-1185 | HTTANWLTAAAAYYVGYLKPTTFMLKYNENGTITDAVDCSQNP |
| | . \| \|.\|\|\|\|.\|\|\|\|\|\|\|\|\|\|\|\|\|.\|\|\|\|\|\|\|\|\|\|\|\|\|\|\| |
| AY274119_tr(CoV_T2_2)/1-1152 | QDI--WGTSAAAYFVGYLKPTTFMLKYDENGTITDAVDCSQNP |
| Wuhan_Node1_tr/5-1185 | LAELKCTLKNFNVDKGIYQTSNFRVSPTQEVVRFPNITNLCPF |
| | \|\|\|\|\|\|..\|.\|..\|\|\|\|\|\|\|\|\|\|\|\| \|. .\|\|\|\|\|\|\|\|\|\|\|\|\| |
| AY274119_tr(CoV_T2_2)/1-1152 | LAELKCSVKSFEIDKGIYQTSNFRVVPSGDVVRFPNITNLCPF |
| Wuhan_Node1_tr/5-1185 | DKVFNATRFPSVYAWERTKISDCVADYTVLYNSTSFSTEKCYG |
| | . \|\|\|\|\|.\|\|\|\|\|\|\|\|\| \|\|\|.\|\|\|\|\|.\|\|\|\|\|\| \|\|\|\|\|\|\|\| |
| AY274119_tr(CoV_T2_2)/1-1152 | GEVFNATKFPSVYAWERKKISNCVADYSVLYNSTFFSTEKCYG |
| Wuhan_Node1_tr/5-1185 | VSPSKLIDLCFTSVYADTFLIRCSEVRQVAPGQTGVIADYNYK |
| | \|\|.\|\| \|\|\|\|..\|\|\|\|.\|... .\|\|\|.\|\|\|\|\|\|\|\|\|\|\|\|\|\|\| |
| AY274119_tr(CoV_T2_2)/1-1152 | VSATKLNDLCFSNVYADSFVVKGDDVRQIAPGQTGVIADYNYK |
| Wuhan_Node1_tr/5-1185 | LPDDFTGCVIAWNTAKQDTGSSGNYNYYYRSHRKTKLKPFERD |
| | \|\|\|\|\| \|\|\|.\|\|\|\| . \|. \|.\|\|\|\|\| \|\| \| \|\|.\|\|\|\|\| |
| AY274119_tr(CoV_T2_2)/1-1152 | LPDDFMGCVLAWNTRNIDATSTGNYNYKYRYLRHGKLRPFERD |
| Wuhan_Node1_tr/5-1185 | LSSDECSPDGKPCTPPAFNGVRGFNCYFTLSTYDFNPNVPVEY |
| | .\|. \|\|\|\|\|\|\|\|\|\|\| .\|\|\| \|. \|.\| . \| |
| AY274119_tr(CoV_T2_2)/1-1152 | ISNVPFSPDGKPCTPPA------LNCYWPLNDYGFYTTTGIGY |
| Wuhan_Node1_tr/5-1185 | QATRVVVLSFELLNAPATVCGPKLSTQLVKNQCVNFNFNGLKG |
| | \|.\|\|\|\|\|\|\|\|\|\|\|\|\|\|\|\|\|\|\|\|\|\|\|\|.\|.\|\|\|\|\|\|\|\|\|\|\|\| \| |
| AY274119_tr(CoV_T2_2)/1-1152 | QPYRVVVLSFELLNAPATVCGPKLSTDLIKNQCVNFNFNGLTG |
| Wuhan_Node1_tr/5-1185 | TGVLTASSKRFQSFQQFGRDASDFTDSVRDPQTLEILDISPCS |
| | \|\|\|\|\|.\|\|\|\|\|\|.\|\|\|\|\|\|\| \|\|\|\|\|\|\|\|\|\|.\| \|\|\|\|\|\|\|\|. |
| AY274119_tr(CoV_T2_2)/1-1152 | TGVLTPSSKRFQPFQQFGRDVSDFTDSVRDPKTSEILDISPCA |
| Wuhan_Node1_tr/5-1185 | FGGVSVITPGTNTSSEVAVLYQDVNCTDVPTAIHADQLTPAWR |
| | \|\|\|\|\|\|\|\|\|\|\|\|.\|\|\|\|\|\|\|\|\|\|\|\|\|\|\|\|.\|\|\|\|\|\|\|\|\|\|\|\|\| |
| AY274119_tr(CoV_T2_2)/1-1152 | FGGVSVITPGTNASSEVAVLYQDVNCTDVSTAIHADQLTPAWR |
| Wuhan_Node1_tr/5-1185 | VYSTGVNVFQTQAGCLIGAEHVNASYECDIPIGAGICASYHTA |
| | .\|\|\|\| \|\|\|\|\|\|\|\|\|\|\|\|\|\|\|\|..\|\|\|\|\|\|\|\|\|\|\|\|\|\|\|\|\|\| |
| AY274119_tr(CoV_T2_2)/1-1152 | IYSTGNNVFQTQAGCLIGAEHVDTSYECDIPIGAGICASYHTV |
| Wuhan_Node1_tr/5-1185 | SNSPRILRSTGQKSIVAYTMSLGAENSIAYANNSIAIPTNFSI |
| | \| .\|\|\|\|.\|\|\|\|\|\|\|\|\|\|\|\|\|..\|\|\|\|.\|\|.\|\|\|\|\|\|\|\|\| |
| AY274119_tr(CoV_T2_2)/1-1152 | S----LLRSTSQKSIVAYTMSLGADSSIAYSNNTIAIPTNFSI |
| Wuhan_Node1_tr/5-1185 | SVTTEVMPVSMAKTSVDCTMYICGDSLECSNLLLQYGSFCTQL |
| | \|.\|\|\|\|\|\|\|\|\|\|\|\|\|\|\|\| \|\|\|\|\|\|\| \|\|.\|\|\|\|\|\|\|\|\|\|\|\|\| |
| AY274119_tr(CoV_T2_2)/1-1152 | SITTEVMPVSMAKTSVDCNMYICGDSTECANLLLQYGSFCTQL |
| Wuhan_Node1_tr/5-1185 | NRALTGIAIEQDKNTQEVFAQVKQMYKTPAIKDFGGFNFSQIL |
| | \|\|\|\|.\|\|\| \|\|\|.\|\|.\|\|\|\|\|\|\|\|\|\|\|\|\|..\| \|\|\|\|\|\|\|\|\|\| |
| AY274119_tr(CoV_T2_2)/1-1152 | NRALSGIAAEQDRNTREVFAQVKQMYKTPTLKYFGGFNFSQIL |
| Wuhan_Node1_tr/5-1185 | PDPSKPTKRSFIEDLLFNKVTLADAGFMKQYGECLGDISARDL |
| | \|\|\| \|\|\|\|\|\|\|\|\|\|\|\|\|\|\|\|\|\|\|\|\|\|\|\|\|\|\|\|\|\|\|\|\|\|.\|\|\|\| |
| AY274119_tr(CoV_T2_2)/1-1152 | PDPLKPTKRSFIEDLLFNKVTLADAGFMKQYGECLGDINARDL |
| Wuhan_Node1_tr/5-1185 | ICAQKFNGLTVLPPLLTDEMIAAYTAALVSGTATAGWTFGAGA |
| | \|\|\|\|\|\|\|\|\|\|\|\|\|\|\|\|\|\|.\|\|\|\|\|\|\|\|\|\|\|\|\|\|\|\|\|\|\|\|\|\|\|\| |
| AY274119_tr(CoV_T2_2)/1-1152 | ICAQKFNGLTVLPPLLTDDMIAAYTAALVSGTATAGWTFGAGA |
| Wuhan_Node1_tr/5-1185 | ALQIPFAMQMAYRFNGIGVTQNVLYENQKQIANQFNKAISQIQ |
| | \|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119_tr(CoV_T2_2)/1-1152 | ALQIPFAMQMAYRFNGIGVTQNVLYENQKQIANQFNKAISQIQ |
| Wuhan_Node1_tr/5-1185 | ESLTTTSTALGKLQDVVNQNAQALNTLVKQLSSNFGAISSVLN |
| | \|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119_tr(CoV_T2_2)/1-1152 | ESLTTTSTALGKLQDVVNQNAQALNTLVKQLSSNFGAISSVLN |
| Wuhan_Node1_tr/5-1185 | DILSRLDKVEAEVQIDRLITGRLQSLQTYVTQQLIRAAEIRAS |
| | \|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119_tr(CoV_T2_2)/1-1152 | DILSRLDKVEAEVQIDRLITGRLQSLQTYVTQQLIRAAEIRAS |
| Wuhan_Node1_tr/5-1185 | ANLAATKMSECVLGQSKRVDFCGKGYHLMSFPQAAPHGVVFLH |
| | \|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119_tr(CoV_T2_2)/1-1152 | ANLAATKMSECVLGQSKRVDFCGKGYHLMSFPQAAPHGVVFLH |
| Wuhan_Node1_tr/5-1185 | VTYVPSQERNFTTAPAICHEGKAYFPREGVFVSNGTSWFITQR |
| | \|\|\|\|\|\|\|\|\|\|\|\|\|\|\|\|\|\|\|\|\|\|\|\|\|\|\|\|\|\|\|\| \|\|\|\|\|\|\|\|\|\| |
| AY274119_tr(CoV_T2_2)/1-1152 | VTYVPSQERNFTTAPAICHEGKAYFPREGVFVFNGTSWFITQR |
| Wuhan_Node1_tr/5-1185 | NFYSPQIITTDNTFVSGNCDVVIGIINNTVYDPLQPELDSFKE |
| | \|\|.\|\|\|\|\|\|\|\|\|\|\|\|\|\|\|\|\|\|\|\|\|\|\|\|\|\|\|\|\|\|\|\|\|\|\|\|\|\|\|\| |
| AY274119_tr(CoV_T2_2)/1-1152 | NFFSPQIITTDNTFVSGNCDVVIGIINNTVYDPLQPELDSFKE |
| Wuhan_Node1_tr/5-1185 | ELDKYFKNHTSPDVDLGDIS |
| | \|\|\|\|\|\|\|\|\|\|\|\|\|\|\|\|\|\|\|\| |
| AY274119_tr(CoV_T2_2)/1-1152 | ELDKYFKNHTSPDVDLGDIS |

Percentage ID = 80.86

### EXAMPLE 5

Alignment of truncated S-protein amino acid sequence of CoV_T2_4 (Wuhan_Node1_tr)
with EPI_ISL_402119

Score = 48450.0
Length of alignment = 1177
Sequence Wuhan_Node1_tr/9-1185 (Sequence length = 1185) (SEQ ID NO: 15)
Sequence EPI_ISL_402119_tr/1-1170 (Sequence length = 1170) (SEQ ID NO: 9)

| | |
|---|---|
| Wuhan_Node1_tr/9-1185 | IFAFFLLSAKANERCGIFTSKPOPKLAQVSSSRRGVYYPDDIFRSDV |
| | .\| \|..\| . . .\| .\|.. \| \| .\| \|\|\|\|\|\|\| .\|\|\| \| |
| EPI_ISL_402119_tr/1-1170 | MFVFLVLLPLVSSQCVNLTTRTQLPPAYTNSFTRGVYYPDKVFRSSV |
| Wuhan_Node1_tr/9-1185 | LHLTQDYFLPFDSNVTRYFSLNANGPDRIVYFDNPIIPFKDGVYFAA |
| | \|\| \|\|\| \|\|\|\| \|\|\|\|.. ... .\| . \|\|\|\|..\|\|.\|\|\|\|\|\|. |
| EPI_ISL_402119_tr/1-1170 | LHSTQDLFLPFFSNVTWFHAIHVSGTNGTKRFDNPVLPFNDGVYFAS |
| Wuhan_Node1_tr/9-1185 | TEKSNVIRGWIFGSTLDNTSQSVIIVNNSTNVIIRVCNFDLCNDPFF |
| | \|\|\|\|\|.\|\|\|\|\|\|\|.\|\|\|. .\|\|..\|\|\|\|.\|\|\|.\|.\|\|.\|..\|\|\|\|\|. |
| EPI_ISL_402119_tr/1-1170 | TEKSNIIRGWIFGTTLDSKTQSLLIVNNATNVVIKVCEFQFCNDPFL |
| Wuhan_Node1_tr/9-1185 | TVSRPTDKHIKTWSIREFAVYQSAFNCTFEYVSKSFLLDVAEKPGNF |
| | \| .\|. \|.\| \|\| \|\| \|\| \|\|\|\|\|\|\|\|..\|\|.\|. \| \|\|\| |
| EPI_ISL_402119_tr/1-1170 | GVY--YHKNNKSWMESEFRVYSSANNCTFEYVSQPFLMDLEGKQGNF |
| Wuhan_Node1_tr/9-1185 | KHLREFVFKNVDGFLNVYSTYKPINVVSGLPTGFSVLKPILKLPLGI |
| | \|.\|\|\|\|\|\|\|\|.\|\|....\|\| \|\|\|.\| .\|\| \|\|\| \| \|.. \|\|.\|\| |
| EPI_ISL_402119_tr/1-1170 | KNLREFVFKNIDGYFKIYSKHTPINLVRDLPQGFSALEPLVDLPIGI |
| Wuhan_Node1_tr/9-1185 | NITSFRVLLTMFRGDPTPGHTTANWLTAAAAYYVGYLKPTTFMLKYN |
| | \|\|\| \|. \|\|.. \|. \|\|\|.... \| ...\|\|\|\|\|\|\|\|.\| \|\|.\|\|\|\| |
| EPI_ISL_402119_tr/1-1170 | NITRFQTLLALHRSYLTPGDSSSGWTAGAAAYYVGYLQPRTFLLKYN |
| Wuhan_Node1_tr/9-1185 | ENGTITDAVDCSQNPLAELKCTLKNFNVDKGIYQTSNFRVSPTQEVV |
| | \|\|\|\|\|\|\|\|\|\|\|. .\|\|.\| \|\|\|\|\|.\| \|.\|\|\|\|\|\|\|\|\|\|\| \|\|. .\| |
| EPI_ISL_402119_tr/1-1170 | ENGTITDAVDCALDPLSETKCTLKSFTVEKGIYQTSNFRVQPTESIV |
| Wuhan_Node1_tr/9-1185 | RFPNITNLCPFDKVFNATRFPSVYAWERTKISDCVADYTVLYNSTSF |
| | \|\|\|\|\|\|\|\|\|\|\|. \|\|\|\|\|\|\|.\|\|\|\|\|.\| .\|\|.\|\|\|\|\|.\|\|\|\|\|.\|\| |
| EPI_ISL_402119_tr/1-1170 | RFPNITNLCPFGEVFNATRFASVYAWNRKRISNCVADYSVLYNSASF |
| Wuhan_Node1_tr/9-1185 | STFKCYGVSPSKLIDLCFTSVYADTFLIRCSEVRQVAPGQTGVIADY |
| | \|\|\|\|\|\|\|\|\|\|.\|\| \|\|\|\|\|.\|\|\|\|.\|.\|\| \|\|\|\|.\|\|\|\|\|\| \|\|\|\| |
| EPI_ISL_402119_tr/1-1170 | STFKCYGVSPTKLNDLCFTNVYADSFVIRGDEVRQIAPGQTGKIADY |
| Wuhan_Node1_tr/9-1185 | NYKLPDDFTGCVIAWNTAKQDTGSSGNYNYYYRSHRKTKLKPFERDL |
| | \|\|\|\|\|\|\|\|\|\|\|\|\|\|\|\|. . \|. .\|\|\|\|\| \|\| \|\|..\|\|\|\|\|\|\|. |
| EPI_ISL_402119_tr/1-1170 | NYKLPDDFTGCVIAWNSNNLDSKVGGNYNYLYRLFRKSNLKPFERDI |
| Wuhan_Node1_tr/9-1185 | SSDECSPDGKPCTPPAFNGVRGFNCYFTLSTYDFNPNVPVEYQATRV |
| | \|.. ... \|\| \|\|\| \|\|\|\|\|\| \| .\|.\|.\| \| \|\|. \|\| |
| EPI_ISL_402119_tr/1-1170 | STEIYQAGSTPC-----NGVEGFNCYFPLQSYGFQPTNGVGYQPYRV |
| Wuhan_Node1_tr/9-1185 | VVLSFELLNAPATVCGPKLSTQLVKNQCVNFNFNGLKGTGVLTASSK |
| | \|\|\|\|\|\|\|\|.\|\|\|\|\|\|\|\|\| \|\|.\|\|\|\|.\|\|\|\|\|\|\|\|\| \|\|\|\|\|\| \|.\| |
| EPI_ISL_402119_tr/1-1170 | VVLSFELLHAPATVCGPKKSTNLVKNKCVNFNFNGLTGTGVLTESNK |
| Wuhan_Node1_tr/9-1185 | RFQSFQQFGRDASDFTDSVRDPQTLEILDISPCSFGGVSVITPGTNT |
| | .\| .\|\|\|\|\|\|\| .\| \|\|..\|\|\|\|\|\|\|\|\|\|\|.\|\|\|\|\|\|\|\|\|\|\|\|\|\|\|\| |
| EPI_ISL_402119_tr/1-1170 | KFLPFQQFGRDIADTTDAVRDPQTLEILDITPCSFGGVSVITPGTNT |
| Wuhan_Node1_tr/9-1185 | SSEVAVLYQDVNCTDVPTAIHADQLTPAWRVYSTGVNVFQTQAGCLI |
| | \|..\|\|\|\|\|\|\|\|\|\|\|.\|\| \|\|\|\|\|\|\|\|\|.\|\|\|\|\|\|\| \|\|\|\|\|.\|\|\|\|\| |
| EPI_ISL_402119_tr/1-1170 | SNQVAVLYQDVNCTEVPVAIHADQLTPTWRVYSTGSNVFQTRAGCLI |
| Wuhan_Node1_tr/9-1185 | GAEHVNASYECDIPIGAGICASYHTASNSPRILRSTGQKSIVAYTMS |
| | \|\|\|\|\|\| \|\|\|\|\|\|\|\|\|\|\|\|\|\|\|\|.\| .\|\|\|\| \|\| . .\|\|.\|\|\|\|\| |
| EPI_ISL_402119_tr/1-1170 | GAEHVNNSYECDIPIGAGICASYQTQTNSPRRARSVASQSIIAYTMS |
| Wuhan_Node1_tr/9-1185 | LGAENSIAYANNSIAIPTNFSISVTTEVMPVSMAKTSVDCTMYICGD |
| | \|\|\|\|\|\|.\|\|.\|\|\|\|\|\|\|\|\|\|.\|\|\|\|\|\|..\|\|\|\|.\|\|\|\|\|\|\|\|\|\|\|\|\| |
| EPI_ISL_402119_tr/1-1170 | LGAENSVAYSNNSIAIPTNFTISVTTEILPVSMTKTSVDCTMYICGD |
| Wuhan_Node1_tr/9-1185 | SLECSNLLLQYGSFCTQLNRALTGIAIEQDKNTQEVFAQVKQMYKTP |
| | \| \|\|\|\|\|\|\|\|\|\|\|\|\|\|\|\|\|\|\|\|\|\|\|\|.\|\|\|\|\|\|\|\|\|\|\|\|\|\|\|.\|\|\|\| |
| EPI_ISL_402119_tr/1-1170 | STECSNLLLQYGSFCTQLNRALTGIAVEQDKNTQEVFAQVKQIYKTP |
| Wuhan_Node1_tr/9-1185 | AIKDFGGFNFSQILPDPSKPTKRSFIEDLLFNKVTLADAGFMKQYGE |
| | .\|\|\|\|\|\|\|\|\|\|\|\|\|\|\|\|\|\|\|.\|\|\|\|\|\|\|\|\|\|\|\|\|\|\|\|\|\|\|\|.\|\|\|\|. |
| EPI_ISL_402119_tr/1-1170 | PIKDFGGFNFSQILPDPSKPSKRSFIEDLLFNKVTLADAGFIKQYGD |
| Wuhan_Node1_tr/9-1185 | CLGDISARDLICAQKFNGLTVLPPLLTDEMIAAYTAALVSGTATAGW |
| | \|\|\|\|\|.\|\|\|\|\|\|\|\|\|\|\|\|\|\|\|\|\|\|\|\|\|\|\|\|\|\| \|\|.\|\|..\|\| \|.\|\| |
| EPI_ISL_402119_tr/1-1170 | CLGDIAARDLICAQKFNGLTVLPPLLTDEMIAQYTSALLAGTITSGW |
| Wuhan_Node1_tr/9-1185 | TFGAGAALQIPFAMQMAYRFNGIGVTQNVLYENQKQIANQFNKAISQ |
| | \|\|\|\|\|\|\|\|\|\|\|\|\|\|\|\|\|\|\|\|\|\|\|\|\|\|\|\|\|\|\|\|\|\|\| \|\|\|\|\|\| \|\|.. |
| EPI_ISL_402119_tr/1-1170 | TFGAGAALQIPFAMQMAYRFNGIGVTQNVLYENQKLIANQFNSAIGK |
| Wuhan_Node1_tr/9-1185 | IQESLTTTSTALGKLQDVVNQNAQALNTLVKQLSSNFGAISSVLNDI |
| | \|\|.\|\|..\|..\|\|\|\|\|\|\|\|\|\|\|\|\|\|\|\|\|\|\|\|\|\|\|\|\|\|\|\|\|\|\|\|\|\|\|\|\| |
| EPI_ISL_402119_tr/1-1170 | IQDSLSSTASALGKLQDVVNQNAQALNTLVKQLSSNFGAISSVLNDI |
| Wuhan_Node1_tr/9-1185 | LSRLDKVEAEVQIDRLITGRLQSLQTYVTQQLIRAAEIRASANLAAT |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| EPI_ISL_402119_tr/1-1170 | LSRLDKVEAEVQIDRLITGRLQSLQTYVTQQLIRAAEIRASANLAAT |
| Wuhan_Node1_tr/9-1185 | KMSECVLGQSKRVDFCGKGYHLMSFPQAAPHGVVFLHVTYVPSQERN |
| | \|\|\|\|\|\|\|\|\|\|\|\|\|\|\|\|\|\|\|\|\|\|\|\|\|\|\|.\|\|\|\|\|\|\|\|\|\|\|\|\|\|.\|\|.\| |
| EPI_ISL_402119_tr/1-1170 | KMSECVLGQSKRVDFCGKGYHLMSFPQSAPHGVVFLHVTYVPAQEKN |
| Wuhan_Node1_tr/9-1185 | FTTAPAICHEGKAYFPREGVFVSNGTSWFITQRNFYSPQIITTDNTF |
| | \|\|\|\|\|\|\|\|\|.\|\|\| \|\|\|\|\|\|\|\|\|\|\|\| \|\|.\|\|\|\|\|\| \|\|\|\|\|\|\|\|\|\| |
| EPI_ISL_402119_tr/1-1170 | FTTAPAICHDGKAHFPREGVFVSNGTHWFVTQRNFYEPQIITTDNTF |
| Wuhan_Node1_tr/9-1185 | VSGNCDVVIGIINNTVYDPLQPELDSFKEELDKYFKNHTSPDVDLGD |
| | \|\|\|\|\|\|\|\|\|\|\|.\|\|\|\|\|\|\|\|\|\|\|\|\|\|\|\|\|\|\|\|\|\|\|\|\|\|\|\|\|\|\|\|\|\|\| |
| EPI_ISL_402119_tr/1-1170 | VSGNCDVVIGIVNNTVYDPLQPELDSFKEELDKYFKNHTSPDVDLGD |
| Wuhan_Node1_tr/9-1185 | IS |
| | \|\| |
| EPI_ISL_402119_tr/1-1170 | IS |

Percentage ID = 77.49

### EXAMPLE 6

Alignment of S-protein RBD amino acid sequence of CoV_T2_7 (Wuhan_Node1_RBD) with
AY274119

Score = 8170.0
Length of alignment = 219
Sequence Wuhan_Node1_RBD/1-219 (Sequence length = 219) (SEQ ID NO: 17)
Sequence AY274119_RBD/1-213 (Sequence length = 213) (SEQ ID NO: 5)

| | |
|---|---|
| Wuhan_Node1_RBD/1-219 | RVSPTQEVVRFPNITNLCPFDKVFNATRFPSVYAWERTKISDCVADYTVL |
| | \|\| \|. .\|\|\|\|\|\|\|\|\|\|\|\|\|. \|\|\|\|\|.\|\|\|\|\|\|\|\|\| \|\|\|.\|\|\|\|\|.\|\| |
| AY274119_RBD/1-213 | RVVPSGDVVRFPNITNLCPFGEVFNATKFPSVYAWERKKISNCVADYSVL |
| Wuhan_Node1_RBD/1-219 | YNSTSFSTFKCYGVSPSKLIDLCFTSVYADTFLIRCSEVRQVAPGQTGVI |
| | \|\|\|\| \|\|\|\|\|\|\|\|\|\|..\|\| \|\|\|\|..\|\|\|\|.\|... .\|\|\|.\|\|\|\|\|\|\|\| |
| AY274119_RBD/1-213 | YNSTFFSTFKCYGVSATKLNDLCFSNVYADSFVVKGDDVRQIAPGQTGVI |
| Wuhan_Node1_RBD/1-219 | ADYNYKLPDDFTGCVIAWNTAKQDTGSSGNYNYYYRSHRKTKLKPFERDL |
| | \|\|\|\|\|\|\|\|\|\|\| \|\|\|.\|\|\|\| . \|. \|.\|\|\|\|\| \|\| \| \|\|.\|\|\|\|\|. |
| AY274119_RBD/1-213 | ADYNYKLPDDFMGCVLAWNTRNIDATSTGNYNYKYRYLRHGKLRPFERDI |
| Wuhan_Node1_RBD/1-219 | SSDECSPDGKPCTPPAFNGVRGFNCYFTLSTYDFNPNVPVEYQATRVVVL |
| | \|. \|\|\|\|\|\|\|\|\|\|\| .\|\|\| \|. \|.\| . \|\|. \|\|\|\|\| |
| AY274119_RBD/1-213 | SNVPFSPDGKPCTPPA------LNCYWPLNDYGFYTTTGIGYQPYRVVVL |
| Wuhan_Node1_RBD/1-219 | SFELLNAPATVCGPKLSTQ |
| | \|\|\|\|\|\|\|\|\|\|\|\|\|\|\|\|\|\|. |
| AY274119_RBD/1-213 | SFELLNAPATVCGPKLSTD |

Percentage ID = 70.32

### EXAMPLE 7

Alignment of S-protein RBD amino acid sequence of CoV_T2_7 (Wuhan_Node1_RBD) with
EPI_ISL_402119

Score = 8150.0
Length of alignment = 219
Wuhan Nodel RBD/1-219 (Sequence length = 219) (SEQ ID NO: 17)
Sequence Wuhan_Node1_RBD/1-219 (Sequence length = 219) (SEQ ID NO: 17)
Sequence EPI_ISL_402119_RBD/1-214 (Sequence length = 214) (SEQ ID NO: 11)

| | |
|---|---|
| Wuhan_Node1_RBD/1-219 | RVSPTQEVVRFPNITNLCPFDKVFNATRFPSVYAWERTKISDCVADY |
| | \|\| \|\|. .\|\|\|\|\|\|\|\|\|\|\|\|. \|\|\|\|\|\|\|.\|\|\|\|\|.\| .\|\|.\|\|\|\|\| |
| EPI_ISL_402119_RBD/1-214 | RVQPTESIVRFPNITNLCPFGEVFNATRFASVYAWNRKRISNCVADY |
| Wuhan_Node1_RBD/1-219 | TVLYNSTSFSTFKCYGVSPSKLIDLCFTSVYADTFLIRCSEVRQVAP |
| | .\|\|\|\|\|.\|\|\|\|\|\|\|\|\|\|\|\|.\|\| \|\|\|\|\|.\|\|\|\|.\|.\|\| \|\|\|\|.\|\| |
| EPI_ISL_402119_RBD/1-214 | SVLYNSASFSTFKCYGVSPTKLNDLCFTNVYADSFVIRGDEVRQIAP |
| Wuhan_Node1_RBD/1-219 | GQTGVIADYNYKLPDDFTGCVIAWNTAKQDTGSSGNYNYYYRSHRKT |
| | \|\|\|\| \|\|\|\|\|\|\|\|\|\|\|\|\|\|\|\|\|\|\|\|. . \|. .\|\|\|\|\| \|\| \|\|. |
| EPI_ISL_402119_RBD/1-214 | GQTGKIADYNYKLPDDFTGCVIAWNSNNLDSKVGGNYNYLYRLFRKS |
| Wuhan_Node1_RBD/1-219 | KLKPFERDLSSDECSPDGKPCTPPAFNGVRGFNCYFTLSTYDFNPNV |
| | .\|\|\|\|\|\|\|.\|.. ... \|\| \|\|\| \|\|\|\|\|\| \| .\|.\|.\| |
| EPI_ISL_402119_RBD/1-214 | NLKPFERDISTEIYQAGSTPC-----NGVEGFNCYFPLQSYGFQPTN |
| Wuhan_Node1_RBD/1-219 | PVEYQATRVVVLSFELLNAPATVCGPKLSTQ |
| | \| \|\|. \|\|\|\|\|\|\|\|\|\|.\|\|\|\|\|\|\|\|\| \|\|. |
| EPI_ISL_402119_RBD/1-214 | GVGYQPYRVVVLSFELLHAPATVCGPKKSTN |

Percentage ID = 70.32

### EXAMPLE 8

pEVAC Expression Vector

FIG. 3 shows a map of the pEVAC expression vector. The sequence of the multiple cloning site of the vector is given below, followed by its entire nucleotide sequence.

Sequence of pEVAC Multiple Cloning Site (MCS) (SEQ ID NO: 19):

```
                                                                                PstI KpnI SalI
pEVAC 1301 ACAGACTGTT CCTTTCCATG GGTCTTTTCT GCAGTCACCG TCGGTACCGT
                                        BclI XbaI BamHI NotI BglII
pEVAC 1351 CGACACGTGT GATCATCTAG AGGATCCGCG GCCGCAGATC T
Entire Sequence of pEVAC (SEQ ID NO: 20):
```

| | | |
|---|---|---|
| CMV-IE-E/P: | 248-989 | CMV immediate early 1 enhancer/promoter |
| KanR: | 3445-4098 | Kanamycin resistance |
| SD: | 990-1220 | Splice donor |
| SA: | 1221-1343 | Splice acceptor |
| Tbgh : | 1392-1942 | Terminator signal from bovine growth hormone |
| pUC-ori : | 2096-2769 | pUC-plasmid origin of replication |

| | |
|---|---|
| 1 | TCGCGCGTTT CGGTGATGAC GGTGAAAACC TCTGACACAT GCAGCTCCCG |
| 51 | GAGACGGTCA CAGCTTGTCT GTAAGCGGAT GCCGGGAGCA GACAAGCCCG |
| 101 | TCAGGGCGCG TCAGCGGGTG TTGGCGGGTG TCGGGGCTGG CTTAACTATG |
| 151 | CGGCATCAGA GCAGATTGTA CTGAGAGTGC ACCATATGCG GTGTGAAATA |
| 201 | CCGCACAGAT GCGTAAGGAG AAAATACCGC ATCAGATTGG CTATTGGCCA |
| 251 | TTGCATACGT TGTATCCATA TCATAATATG TACATTTATA TTGGCTCATG |
| 301 | TCCAACATTA CCGCCATGTT GACATTGATT ATTGACTAGT TATTAATAGT |
| 351 | AATCAATTAC GGGGTCATTA GTTCATAGCC CATATATGGA GTTCCGCGTT |
| 401 | ACATAACTTA CGGTAAATGG CCCGCCTGGC TGACCGCCCA ACGACCCCCG |
| 451 | CCCATTGACG TCAATAATGA CGTATGTTCC CATAGTAACG CCAATAGGGA |
| 501 | CTTTCCATTG ACGTCAATGG GTGGAGTATT TACGGTAAAC TGCCCACTTG |
| 551 | GCAGTACATC AAGTGTATCA TATGCCAAGT ACGCCCCCTA TTGACGTCAA |
| 601 | TGACGGTAAA TGGCCCGCCT GGCATTATGC CCAGTACATG ACCTTATGGG |
| 651 | ACTTTCCTAC TTGGCAGTAC ATCTACGTAT TAGTCATCGC TATTACCATG |
| 701 | GTGATGCGGT TTTGGCAGTA CATCAATGGG CGTGGATAGC GGTTTGACTC |
| 751 | ACGGGGATTT CCAAGTCTCC ACCCCATTGA CGTCAATGGG AGTTTGTTTT |
| 801 | GGCACCAAAA TCAACGGGAC TTTCCAAAAT GTCGTAACAA CTCCGCCCCA |
| 851 | TTGACGCAAA TGGGCGGTAG GCGTGTACGG TGGGAGGTCT ATATAAGCAG |
| 901 | AGCTCGTTTA GTGAACCGTC AGATCGCCTG GAGACGCCAT CCACGCTGTT |
| 951 | TTGACCTCCA TAGAAGACAC CGGGACCGAT CCAGCCTCCA TCGGCTCGCA |
| 1001 | TCTCTCCTTC ACGCGCCCGC CGCCCTACCT GAGGCCGCCA TCCACGCCGG |
| 1051 | TTGAGTCGCG TTCTGCCGCC TCCCGCCTGT GGTGCCTCCT GAACTGCGTC |
| 1101 | CGCCGTCTAG GTAAGTTTAA AGCTCAGGTC GAGACCGGGC CTTTGTCCGG |
| 1151 | CGCTCCCTTG GAGCCTACCT AGACTCAGCC GGCTCTCCAC GCTTTGCCTG |
| 1201 | ACCCTGCTTG CTCAACTCTA GTTAACGGTG GAGGGCAGTG TAGTCTGAGC |
| 1251 | AGTACTCGTT GCTGCCGCGC GCGCCACCAG ACATAATAGC TGACAGACTA |
| 1301 | ACAGACTGTT CCTTTCCATG GGTCTTTTCT GCAGTCACCG TCGGTACCGT |
| 1351 | CGACACGTGT GATCATCTAG AGGATCCGCG GCCGCAGATC TGCTGTGCCT |
| 1401 | TCTAGTTGCC AGCCATCTGT TGTTTGCCCC TCCCCCGTGC CTTCCTTGAC |
| 1451 | CCTGGAAGGT GCCACTCCCA CTGTCCTTTC CTAATAAAAT GAGGAAATTG |
| 1501 | CATCGCATTG TCTGAGTAGG TGTCATTCTA TTCTGGGGGG TGGGGTGGGG |
| 1551 | CAGGACAGCA AGGGGGAGGA TTGGGAAGAC AATAGCAGGC ATGCTGGGGA |
| 1601 | TGCGGTGGGC TCTATGGCTA CCCAGGTGCT GAAGAATTGA CCCGGTTCCT |
| 1651 | CCTGGGCCAG AAAGAAGCAG GCACATCCCC TTCTCTGTGA CACACCCTGT |
| 1701 | CCACGCCCCT GGTTCTTAGT TCCAGCCCCA CTCATAGGAC ACTCATAGCT |
| 1751 | CAGGAGGGCT CCGCCTTCAA TCCCACCCGC TAAAGTACTT GGAGCGGTCT |
| 1801 | CTCCCTCCCT CATCAGCCCA CCAAACCAAA CCTAGCCTCC AAGAGTGGGA |
| 1851 | AGAAATTAAA GCAAGATAGG CTATTAAGTG CAGAGGGAGA GAAAATGCCT |
| 1901 | CCAACATGTG AGGAAGTAAT GAGAGAAATC ATAGAATTTT AAGGCCATGA |
| 1951 | TTTAAGGCCA TCATGGCCTT AATCTTCCGC TTCCTCGCTC ACTGACTCGC |
| 2001 | TGCGCTCGGT CGTTCGGCTG CGGCGAGCGG TATCAGCTCA CTCAAAGGCG |
| 2051 | GTAATACGGT TATCCACAGA ATCAGGGGAT AACGCAGGAA AGAACATGTG |
| 2101 | AGCAAAAGGC CAGCAAAAGG CCAGGAACCG TAAAAAGGCC GCGTTGCTGG |
| 2151 | CGTTTTTCCA TAGGCTCCGC CCCCCTGACG AGCATCACAA AAATCGACGC |
| 2201 | TCAAGTCAGA GGTGGCGAAA CCCGACAGGA CTATAAAGAT ACCAGGCGTT |
| 2251 | TCCCCCTGGA AGCTCCCTCG TGCGCTCTCC TGTTCCGACC CTGCCGCTTA |
| 2301 | CCGGATACCT GTCCGCCTTT CTCCCTTCGG GAAGCGTGGC GCTTTCTCAT |
| 2351 | AGCTCACGCT GTAGGTATCT CAGTTCGGTG TAGGTCGTTC GCTCCAAGCT |
| 2401 | GGGCTGTGTG CACGAACCCC CCGTTCAGCC CGACCGCTGC GCCTTATCCG |
| 2451 | GTAACTATCG TCTTGAGTCC AACCCGGTAA GACACGACTT ATCGCCACTG |
| 2501 | GCAGCAGCCA CTGGTAACAG GATTAGCAGA GCGAGGTATG TAGGCGGTGC |
| 2551 | TACAGAGTTC TTGAAGTGGT GGCCTAACTA CGGCTACACT AGAAGAACAG |
| 2601 | TATTTGGTAT CTGCGCTCTG CTGAAGCCAG TTACCTTCGG AAAAAGAGTT |
| 2651 | GGTAGCTCTT GATCCGGCAA ACAAACCACC GCTGGTAGCG GTGGTTTTTT |
| 2701 | TGTTTGCAAG CAGCAGATTA CGCGCAGAAA AAAAGGATCT CAAGAAGATC |
| 2751 | CTTTGATCTT TTCTACGGGG TCTGACGCTC AGTGGAACGA AAACTCACGT |
| 2801 | TAAGGGATTT TGGTCATGAG ATTATCAAAA AGGATCTTCA CCTAGATCCT |
| 2851 | TTTAAATTAA AAATGAAGTT TTAAATCAAT CTAAAGTATA TATGAGTAAA |
| 2901 | CTTGGTCTGA CAGTTACCAA TGCTTAATCA GTGAGGCACC TATCTCAGCG |
| 2951 | ATCTGTCTAT TTCGTTCATC CATAGTTGCC TGACTCGGGG GGGGGGGGCG |
| 3001 | CTGAGGTCTG CCTCGTGAAG AAGGTGTTGC TGACTCATAC CAGGCCTGAA |
| 3051 | TCGCCCCATC ATCCAGCCAG AAAGTGAGGG AGCCACGGTT GATGAGAGCT |
| 3101 | TTGTTGTAGG TGGACCAGTT GGTGATTTTG AACTTTTGCT TTGCCACGGA |
| 3151 | ACGGTCTGCG TTGTCGGGAA GATGCGTGAT CTGATCCTTC AACTCAGCAA |
| 3201 | AAGTTCGATT TATTCAACAA AGCCGCCGTC CCGTCAAGTC AGCGTAATGC |
| 3251 | TCTGCCAGTG TTACAACCAA TTAACCAATT CTGATTAGAA AAACTCATCG |
| 3301 | AGCATCAAAT GAAACTGCAA TTTATTCATA TCAGGATTAT CAATACCATA |
| 3351 | TTTTTGAAAA AGCCGTTTCT GTAATGAAGG AGAAAACTCA CCGAGGCAGT |
| 3401 | TCCATAGGAT GGCAAGATCC TGGTATCGGT CTGCGATTCC GACTCGTCCA |
| 3451 | ACATCAATAC AACCTATTAA TTTCCCCTCG TCAAAAATAA GGTTATCAAG |
| 3501 | TGAGAAATCA CCATGAGTGA CGACTGAATC CGGTGAGAAT GGCAAAAGCT |
| 3551 | TATGCATTTC TTTCCAGACT TGTTCAACAG GCCAGCCATT ACGCTCGTCA |
| 3601 | TCAAAATCAC TCGCATCAAC CAAACCGTTA TTCATTCGTG ATTGCGCCTG |
| 3651 | AGCGAGACGA AATACGCGAT CGCTGTTAAA AGGACAATTA CAAACAGGAA |
| 3701 | TCGAATGCAA CCGGCGCAGG AACACTGCCA GCGCATCAAC AATATTTTCA |
| 3751 | CCTGAATCAG GATATTCTTC TAATACCTGG AATGCTGTTT TCCCGGGGAT |
| 3801 | CGCAGTGGTG AGTAACCATG CATCATCAGG AGTACGGATA AAATGCTTGA |
| 3851 | TGGTCGGAAG AGGCATAAAT TCCGTCAGCC AGTTTAGTCT GACCATCTCA |
| 3901 | TCTGTAACAT CATTGGCAAC GCTACCTTTG CCATGTTTCA GAAACAACTC |
| 3951 | TGGCGCATCG GGCTTCCCAT ACAATCGATA GATTGTCGCA CCTGATTGCC |
| 4001 | CGACATTATC GCGAGCCCAT TTATACCCAT ATAAATCAGC ATCCATGTTG |
| 4051 | GAATTTAATC GCGGCCTCGA GCAAGACGTT TCCCGTTGAA TATGGCTCAT |
| 4101 | AACACCCCTT GTATTACTGT TTATGTAAGC AGACAGTTTT ATTGTTCATG |
| 4151 | ATGATATATT TTTATCTTGT GCAATGTAAC ATCAGAGATT TTGAGACACA |
| 4201 | ACGTGGCTTT CCCCCCCCCC CCATTATTGA AGCATTTATC AGGGTTATTG |
| 4251 | TCTCATGAGC GGATACATAT TTGAATGTAT TTAGAAAAAT AAACAAATAG |
| 4301 | GGGTTCCGCG CACATTTCCC CGAAAAGTGC CACCTGACGT CTAAGAAACC |
| 4351 | ATTATTATCA TGACATTAAC CTATAAAAAT AGGCGTATCA CGAGGCCCTT |
| 4401 | TCGTC |

### EXAMPLE 9

Common Amino Acid Differences of Wuhan Node1 RBD (CoV_T2_7) Amino Acid Sequence (SEQ ID NO: 17) with AY274119 RBD (CoV_T2_5) (SEQ ID NO:5) and EPI ISL 402119 RBD (CoV_T2_6) (SEQ ID NO: 11) Amino Acid Sequences

FIG. 4 shows Wuhan_Node1_RBD (CoV_T2_7) amino acid sequence (SEQ ID NO: 17) with amino acid residue differences highlighted in bold and underline from the respective alignments with AY274119_RBD (COV_T2_5) (SEQ ID NO:5) and EPI_ISL_402119_RBD (CoV_T2_6) (SEQ ID NO:11) amino acid sequences (Examples 6 and 7, respectively).

The amino acid residue differences from the two alignments are listed in the table below (the numbering of residue positions corresponds to positions of the Wuhan_Node1_RBD (CoV_T2_7) (SEQ ID NO:17) amino acid sequence. The common differences from the two alignments are at amino acid residues: 3, 6, 7, 21, 22, 38, 42, 48, 67, 70, 76, 81, 83, 86, 87, 92, 121, 122, 123, 125, 126, 128, 134, 137, 138, 141, 150, 152, 153, 154, 155, 167, 171, 178, 180, 181, 183, 185, 187, 188, 189, 191, 194, 195, 219 (shown with grey highlighting in FIG. 4, and in the table below):

| Wuhan_Node1_RBD | Amino acid residue | Amino acid residue |
|---|---|---|
| (CoV_T2_7) residue | difference vs | difference vs |
| position | AY274119_RBD | EPI_ISL_402119_RBD |
| 3 | S | S |
| 5 | T | — |
| 6 | Q | Q |
| 7 | E | E |
| 8 | — | V |
| 21 | D | D |
| 22 | K | K |
| 28 | R | — |
| 30 | — | P |
| 36 | — | E |
| 38 | T | T |
| 39 | — | K |
| 42 | D | D |
| 48 | T | T |
| 54 | — | T |
| 55 | S | — |
| 66 | P | — |
| 67 | S | S |
| 70 | I | I |
| 75 | T | — |
| 76 | S | S |
| 81 | T | T |
| 83 | L | L |
| 84 | I | — |
| 85 | R | — |
| 86 | C | C |
| 87 | S | S |
| 88 | E | — |
| 92 | V | V |
| 99 | — | V |
| 112 | T | — |
| 116 | I | — |
| 120 | — | T |
| 121 | A | A |
| 122 | K | K |
| 123 | Q | Q |
| 125 | T | T |
| 126 | G | G |
| 127 | — | S |
| 128 | S | S |
| 134 | Y | Y |
| 137 | S | S |
| 138 | H | H |
| 140 | K | — |
| 141 | T | T |
| 142 | — | K |
| 144 | K | — |
| 150 | L | L |
| 152 | S | S |
| 153 | D | D |
| 154 | E | E |
| 155 | C | C |
| 156 | — | S |
| 157 | — | P |
| 158 | — | D |
| 159 | — | G |
| 160 | — | K |
| 163 | — | T |
| 164 | — | P |
| 165 | — | P |
| 166 | — | A |
| 167 | F | F |
| 168 | N | — |
| 169 | G | — |
| 170 | V | — |
| 171 | R | R |
| 172 | G | — |
| 173 | F | — |
| 177 | F | — |
| 178 | T | T |
| 180 | S | S |
| 181 | T | T |
| 183 | D | D |
| 185 | N | N |
| 186 | P | — |
| 187 | N | N |
| 188 | V | V |
| 189 | P | P |
| 190 | V | — |
| 191 | E | E |
| 194 | A | A |
| 195 | T | T |
| 206 | — | N |
| 216 | — | L |
| 219 | Q | Q |

Amino acid insertions are at positions 167-172 (compared to AY274119_RBD), and 163-167 (compared to EPI_ISL_402119_RBD) (shown boxed in FIG. 4).

### EXAMPLE 9

Immune Response Induced by DNA Vaccine Encoding “panS” Antigen

Mice (n=6) were immunised with DNA encoding a “panS” antigen according to an embodiment of the invention (Wuhan_Node1 (CoV_T2_1), nucleic acid of SEQ ID NO:13, encoding full length S-protein of amino acid SEQ ID NO:14), full-length S gene from SARS-Cov-1, or full-length S gene from SARS-CoV-2.

Antibodies in serum obtained from the mice were compared for their ability to bind wild-type antigens through FACS.

FIG. 5 shows dose response curves of antibody binding to SARS-CoV-1 (A) or SARS-CoV-2 (B) full length Spike protein expressed on HEK293T cells. Flow cytometry based cell display assay reported in MFI (Median Fluorescent Intensity).

Serum from mice immunised with either wildtype S gene show weak binding to heterologous protein. In contrast, serum from mice immunised with the “panS” antigen binds to both SARS-CoV-1 and SARS-CoV-2 Spike proteins.

It was concluded that the “panS” antigen induces an immune response that is more cross-reactive than wild-type antigens, indicating protection against future Sarbecovirus outbreaks not conferred by using naturally occurring antigens.

### EXAMPLE 10

### Envelope (E) Protein Vaccine Sequences

FIG. 6 shows an amino acid sequence of the SARS envelope protein (SEQ ID NO:21), and illustrates key features of the sequence:

(SEQ ID NO: 21)
MYSFVSEETG TLIVNSVLLF LAFVVFLLVT LAILTALRLC
AYCCNIVNVS LVKPTVYVYS RVKNLNSSEG VPDLLV

FIG. 7 shows a multiple sequence alignment of coronavirus Envelope (E) protein sequences, comparing sequences for isolates of NL63 and 229E (alpha-coronaviruses), and HKU1, MERS, SARS, and SARS2 (beta-coronaviruses). The alignment shows that the C-terminal end of the E protein for the SARS2 and SARS sequences (beta-coronaviruses of subgenus Sarbeco) includes a deletion, compared with the other sequences, and that the SARS2 E protein sequence includes a deletion, and an Arginine (positively charged) amino acid residue, compared with the SARS sequence.

We have generated novel sequences for the Envelope (E) protein, called COV_E_T2_1 (a designed Sarbecovirus sequence) (SEQ ID NO:22) and COV_E_T2_2 (a designed SARS2 sequence) (SEQ ID NO:23):

>COV_E_T2_1
(SEQ ID NO: 22)
MYSFVSEETG TLIVNSVLLF LAFVVFLLVT LAILTALRLC
AYCCNIVNVS LVKPTFYVYS RVKNLNSSQG VPDLLV
>COV_E_T2 2
(SEQ ID NO: 23)
MYSFVSEETG TLIVNSVLLF LAFVVFLLVT LAILTALRLC
AYCCNIVNVS LVKPTFYVYS RVKNLNSSR- VPDLLV

Alignment of the SARS2 reference E protein sequence in FIG. 7 with these designed sequences highlights that there are four amino acid differences between the SARS2 reference E protein sequence and the COV_E_T2_1 designed sequence (SEQ ID NO:22), and two amino acid differences between the SARS2 reference E protein sequence and the COV_E_T2_2 designed sequence (SEQ ID NO:23) (see the boxed amino acid residues in the amino acid sequence alignment below):

| | |
|---|---|
| SARS2 COV_E_T2_ [symbol] | 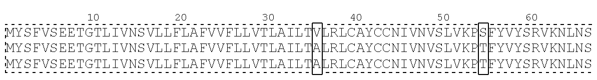 |
| SARS2 COV_E_T2_ [symbol] | 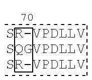 |

The C-terminal sequence of the COV_E_T2_2 sequence is identical to the SARS2 reference sequence. The C-terminal of the E protein is one of the identified epitopes for E-protein, so the amino acid deletion and the substitution with an Arginine residue present in the SARS2 reference sequence (compared with the SARS reference sequence in FIG. 6) have been retained in the COV_E_T2_2 designed sequence. The amino acid differences at the other positions are optimised to maximise induction of an immune response that recognises all Sarbeco viruses.

The amino acid differences are summarised in the table below:

| SARS2 E | SARS2 | | |
|---|---|---|---|
| protein | Reference | COV_E_T2_1 | COV_E_T2_2 |
| residue | Amino acid | Amino acid | Amino acid |
| position | residue | residue | residue |
| 36 | V | A | A |
| 55 | S | T | T |
| 69 | R | Q | R |
| 70 | — | G | — |

In the alignment above, residue 36 of the SARS2 reference sequence is shown as V, but is actually A (as correctly shown in FIG. 7 and SEQ ID NO:21). Alignment of SEQ ID NO:21 with the designed sequences highlights that there are three amino acid differences between the alternative SARS2 reference E protein sequence and the COV_E_T2_1 designed sequence (SEQ ID NO:22), and one amino acid difference between the SARS2 reference E protein sequence and the COV_E_T2_2 designed sequence (SEQ ID NO:23):

| | | |
|---|---|---|
| SARS2 COV_E_T2_1 COV_E_T2_2 | 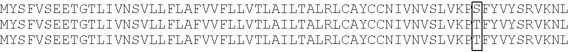 | 65 65 65 |
| SARS2 COV_E_T2_1 COV_E_T2_2 | 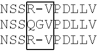 | 75 76 75 |

The amino acid differences are summarised in the table below:

| SARS2 E | SARS2 | | |
|---|---|---|---|
| protein | Reference | COV_E_T2_1 | COV_E_T2_2 |
| residue | Amino acid | Amino acid | Amino acid |
| position | residue | residue | residue |
| 55 | S | T | T |
| 69 | R | Q | R |
| 70 | — | G | — |

### EXAMPLE 11

### Membrane (M) Protein Vaccine Sequences

We have generated novel sequences for the coronavirus membrane (M) protein:
COV_M_T2_1 Sarbecovirus root ancestor (SEQ ID NO:24);
COV_M_T2_2 Epitope optimised version of SARS2 clade ancestor Node88b (D4 removed), SARS2 equivalent of B cell epitope from start and end added, and then T cell epitopes added whilst observing coevolving site constraints (SEQ ID NO:25).

The amino acid sequences of these designed sequences are:

>COV_M_T2_1/1-221 Sarbeco_M_root:
(SEQ ID NO: 24)
MADNGTITVE ELKQLLEQWN LVIGFLFLAW IMLLQFAYSN RNRFLYIIKL VFLWLLWPVT
LACFVLAAVY RINWVTGGIA IAMACIVGLM WLSYFVASFR LFARTRSMWS FNPETNILLN
VPLRGTILTR PLMESELVIG AVIIRGHLRM AGHSLGRCDI KDLPKEITVA TSRTLSYYKL
GASQRVGTDS GFAAYNRYRI GNYKLNTDHA GSNDNIALLV Q
>COV_M_T2_2/1-222 Sarbeco_M_Node88b_epitope_optimised:
(SEQ ID NO: 25)
MADSNGTITV EELKKLLEQW NLVIGFLFLT WICLLQFAYS NRNRFLYIIK LIFLWLLWPV
TLACFVLAAV YRINWVTGGI AIAMACIVGL MWLSYFVASF RLFARTRSMW SENPETNILL
NVPLRGSIIT RPLMESELVI GAVILRGHLR MAGHSLGRCD IKDLPKEITV ATSRTLSYYK
LGASQRVASD SGFAVYNRYR IGNYKLNTDH SSSSDNIALL VQ

Alignment of the following SARS2 reference M protein sequence (SEQ ID NO:26) with the designed sequences is shown in FIG. 8. The reference M protein sequence is:

>COV_M_T1_1/1-222 NC_045512.2 SARS2 reference sequence:
(SEQ ID NO: 26)
MADSNGTITV EELKKLLEQW NLVIGFLFLT WICLLQFAYA NRNRFLYIIK LIFLWLLWPV
TLACFVLAAV YRINWITGGI AIAMACLVGL MWLSYFIASF RLFARTRSMW SENPETNILL
NVPLHGTILT RPLLESELVI GAVILRGHLR IAGHHLGRCD IKDLPKEITV ATSRTLSYYK
LGASQRVAGD SGFAAYSRYR IGNYKLNTDH SSSSDNIALL VQ

The alignment shown in FIG. 8 highlights the amino acid differences between the SARS2 reference M protein sequence and the COV_M_T2_1 and COV_M_T2_2 designed sequences, as shown in the table below:

| SARS2 M | SARS2 | | |
|---|---|---|---|
| protein | Reference | COV_M_T2_1 | COV_M_T2_2 |
| residue | Amino acid | Amino acid | Amino acid |
| position | residue | residue | residue |
| 4 | S | — | S |
| 15 | K | Q | K |
| 30 | T | A | T |
| 33 | C | M | C |
| 40 | A | S | S |
| 52 | I | V | I |
| 76 | I | V | V |
| 87 | L | I | I |
| 97 | I | V | V |
| 125 | H | R | R |
| 127 | T | T | S |
| 134 | L | M | M |
| 145 | L | I | L |
| 151 | I | M | M |
| 155 | H | S | S |
| 188 | A | G | A |
| 189 | G | T | S |
| 195 | A | A | V |
| 197 | S | N | N |
| 211 | S | A | S |
| 212 | S | G | S |
| 214 | S | N | S |

### EXAMPLE 12

### Clinical Trial Design

The study will consist of thirty SARS-CoV-2 PCR, antibody and T-cell negative healthy human volunteers enrolled for this trial, who agree to self-isolate and report back during the three immunisations, in order to demonstrate safety and immunogenicity.

The first of 3 study Groups will consist of:
Group 1; n=6 dose escalation;
Group 2; 12 healthy human volunteers with the needleless PharmaJet delivery;
Group 3; 12 healthy human volunteers receiving direct intramuscular (IM) administration of DNA to benchmark the results by Martin et al (Vaccine, 2008).

The PharmaJet arm of the trial uses a dose-sparing needleless delivery system, which minimises the barriers to people taking the vaccine. Power calculations are based on an estimated standard deviation of 0.27 log 10 units, using the ELISA data from the SARS clinical Trial (Martin et al, Vaccine, 2008).

Due to the pandemic emergency, primary and secondary endpoints will be analysed when the last patient has completed 3 months following primary immunisation (complete safety data for 28 days, and immunogenicity primary and key secondary endpoints to 3 months).

### Secondary Objective/Endpoints to Assess the Immunogenicity of the Vaccine:

Key immunogenicity endpoints to be analysed and reported at 3 months: Serology (t=0, 14 days, 28 days, 2 months, 3 months). In addition to antigen specific IgM and IgG ELISAs, ADE and ADCC assays will be performed at all time points. Standardised microneutralization assays to measure neutralizing capacity of vaccine antigen-specific antibodies in sera collected pre- and post-immunization at the defined time points.

Antigen-specific T cellular immune responses will be measured at t=0, 14 days, 28 days, 2 months, 3 months). Antigen-specific T cell immune responses will be evaluated in cryopreserved PBMC from vaccinees by proliferation assay (CFSE) and IFN gamma ELISPOT as a preliminary screening of positive responders. A detailed phenotypic analysis of the vaccine-induced T cell responses performed by flow cytometry will follow to determine subpopulations induced by the vaccine candidates [Central memory T-cells (TCM), Effector memory T-cells (TEM) and regulatory T-cells (Treg)] coupled to functional analysis of T cells by intracellular staining for different cytokines (IFN gamma, TNF-α, IL-17, IL-2 and IL-10). Ex vivo nCOV-specific CD8+ and CD4+ T cell subsets, tested for their expression of CD3, CD4, CD8, CD45RA/RO, CD62L, CCR7, CD127, CD25 and nuclear FoxP3, will be identified by multiparametric flow cytometry with fluorochrome-labelled dextramers. If necessary, dextramer analysis will be coupled to a 12-15 day in vitro re-stimulation with vaccine-specific synthetic peptides (20 amino acids overlapped by 12 amino-acids) spanning the Spike(S) protein. Moreover, supernatants of secondary cultures will be also assessed for a large panel of cytokines (IFN-gamma IL-4, IL-5, IL-2, IL-10, IL-13, IL-17, IL-21 and TNF-α) in order to precisely define T cell polarization allowing the identification of T helper subsets and poly-functionality by using the Bio-Plex Pro™ Human Cytokine plex Assay (Biorad).

### EXAMPLE 13

### Further Designed S Protein RBD Sequences.

We have generated further novel S protein RBD sequences by modifying the previous input alignment to our design algorithm: CoV_S_T2_13-COV_S_T2_18. COV_S_T2_13 is the direct output of the design algorithm, and CoV_S_T2_14-COV_S_T2_18 are epitope-enriched versions of COV_S_T2_13.

The amino acid sequences of these designed sequences are:

>COV_S_T2_13
(SEQ ID NO: 27)
RVAPTKEVVR FPNITNLCPF GEVENATRFP SVYAWERKRI SNCVADYSVL YNSTSFSTFK CYGVSPTKLN
DLCFTNVYAD SFVIRGDEVR QIAPGQTGVI ADYNYKLPDD FTGCVIAWNT NNLDSTTGGN YNYLYRSLRK
SKLKPFERDI SSDIYSPGGK PCSGVEGENC YYPLRSYGFF PTNGVGYQPY RVVVLSFELL NAPATVCGPK
LSTD
>COV_S_T2_14
(SEQ ID NO: 28)
RVAPTKEVVR FPNITNLCPF GEVENATKEP SVYAWERKKI SNCVADYSVL YNSTSFSTFK CYGVSPTKLN
DLCFTNVYAD SFVIRGDEVR QIAPGQTGVI ADYNYKLPDD FTGCVIAWNT NNIDSTTGGN YNYLYRSLRK
SKLKPFERDI SSDIYSPGGK PCSGVEGENC YYPLRSYGFF PTNGVGYQPY RVVVLSFELL NAPATVCGPK
LSTD
>COV_S_T2_15
(SEQ ID NO: 29)
RVAPTKEVVR FPNITNLCPF GEVENATREP SVYAWERKRI SNCVADYSVL YNSTFFSTEK CYGVSPTKLN
DLCFSNVYAD SFVIRGDEVR QIAPGQTGVI ADYNYKLPDD FMGCVIAWNT NNLDSTTGGN YNYLYRSLRK
SKLKPFERDI SSDIYSPGGK PCSGVEGENC YYPLRSYGFF PTNGVGYQPY RVVVLSFELL NAPATVCGPK
LSTD
>COV_S_T2_16
(SEQ ID NO: 30)
RVAPTKEVVR FPNITNLCPF GEVENATRFP SVYAWERKRI SNCVADYSVL YNSTSFSTFK CYGVSPTKLN
DLCFTNVYAD SFVIRGDEVR QIAPGQTGKI ADYNYKLPDD FTGCVIAWNT NNLDSTTGGN YNYLYRLFRK
SNLKPFERDI SSDIYQAGST PCSGVEGENC YFPLQSYGFQ PTNGVGYQPY RVVVLSFELL NAPATVCGPK
LSTD
>COV_S_T2_17
(SEQ ID NO: 31)
RVAPTKEVVR FPNITNLCPF GEVENATKFP SVYAWERKKI SNCVADYSVL YNSTSFSTEK CYGVSPTKLN
DLCFTNVYAD SFVIRGDEVR QIAPGQTGVI ADYNYKLPDD FTGCVIAWNT NNIDSTTGGN YNYLYRSLRK
SKLKPFERDI SSDIYSPGGK PCSGVEGENC YYPLRSYGFF PTNGTGYQPY RVVVLSFELL NAPATVCGPK
LSTD
>COV_S_T2_18
(SEQ ID NO: 32)
RVAPTKEVVR FPNITNLCPF GEVENATREP SVYAWERKRI SNCVADYSVL YNSTFFSTEK CYGVSPTKLN
DLCFSNVYAD SFVIRGDEVR QIAPGQTGVI ADYNYKLPDD FMGCVIAWNT NNLDSTTGGN YNYLYRSLRK
SKLKPFERDI SSDIYSPGGK PCSGVEGENC YYPLRSYGFF PTNGTGYQPY RVVVLSFELL NAPATVCGPK
LSTD

Alignment of these sequences with SARS2 Reference sequence (EPI_ISL_402119_RBD (CoV_T2_6) (SEQ ID NO:11)) is shown below (the boxed regions highlight sequence differences in the alignments):

| | | |
|---|---|---|
| SARS2 COV_S_T2_13 COV_S_T2_14 COV_S_T2_15 COV_S_T2_16 COV_S_T2_17 COV_S_T2_18 | 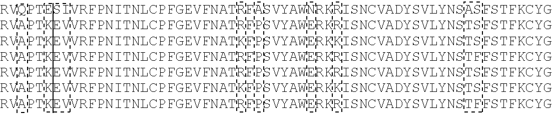 | 63 63 63 63 63 63 63 |
| SARS2 COV_S_T2_13 COV_S_T2_14 COV_S_T2_15 COV_S_T2_16 COV_S_T2_17 COV_S_T2_18 | 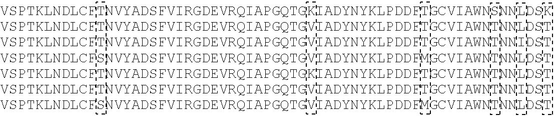 | 126 126 126 126 126 126 126 |
| SARS2 COV_S_T2_13 COV_S_T2_14 COV_S_T2_15 COV_S_T2_16 COV_S_T2_17 COV_S_{'}T2_18 | 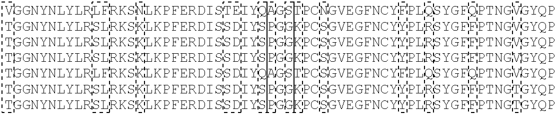 | 189 189 189 189 189 189 189 |
| SARS2 COV_S_T2_13 COV_S_T2_14 COV_S_T2_15 COV_S_T2_16 COV_S_T2_17 COV_S_T2_18 | 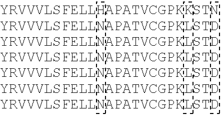 | 214 214 214 214 214 214 214 |

### EXAMPLE 14

Further Designed S Protein RBD Sequences (with Altered Glycosylation Sites)

Masking/de-masking of epitopes has been shown to alter the immune response by masking non-neutralising epitopes, or by de-masking important epitopes in MERS (Du L et. al., Nat. Comm, 2016).

We have prepared additional designed S protein RBD sequences in which we have deleted a glycosylation site of, or introduced a glycosylation site to, the SARS2 RBD sequence. The changes made are illustrated in FIG. 13. The figure shows amino acid sequence of the RBD region. The circled numbers show the positions at which a glycosylation site has been deleted or introduced. Numbers circled in light grey represent deletion of a glycosylation site. Numbers circled in dark grey represent introduction of a glycosylation site. At the position marked by circled number 3, a glycosylation site is present in the SARS wild-type sequence, but absent in the SARS-2 wild-type sequence. This may be important for non-neutralising epitope masking. The introduced glycosylation site is only present in the M8 design.

### Modifications in the RBD:

designs M7 and M9 include a glycosylation site introduced at the position indicated by circled number 4 (residue position 203);
designs M8 and M10 include a deleted glycosylation site at each of the positions indicated by circled numbers 1 and 2 (residue positions 13 and 25, respectively). The M8 design also includes an introduced glycosylation site at the position indicated by circled number 3 (residue position 54).

The amino acid sequences of SARS2 RBD designs M7, M8, M9, and M10 are shown below.

>M7
(SEQ ID NO: 33)
RVQPTESIVR FPNITNLCPF GEVENATRFA SVYAWNRKRI SNCVADYSVL YNSASFSTEK CYGVSPTKLN
DLCFTNVYAD SFVIRGDEVR QIAPGQTGKI ADYNYKLPDD FTGCVIAWNS NNLDSKVGGN YNYLYRLFRK
SNLKPFERDI STEIYQAGST PCNGVEGENC YFPLQSYGFQ PTNGVGYQPY RVVVLSFELL HANATVCGPK
KSTN
>M8
(SEQ ID NO: 34)
RVQPTESIVR FPQITNLCPF GEVFQATRFA SVYAWNRKRI SNCVADYSVL YNSTSFSTEK CYGVSPTKLN
DLCFTNVYAD SFVIRGDEVR QIAPGQTGKI ADYNYKLPDD FTGCVIAWNS NNLDSKVGGN YNYLYRLFRK
SNLKPFERDI STEIYQAGST PCNGVEGENC YFPLQSYGFQ PTNGVGYQPY RVVVLSFELL HAPATVCGPK
KSTN
>M9
(SEQ ID NO: 35)
RVSPTQEVVR FPNITNLCPF DKVFNATRFP SVYAWERTKI SDCVADYTVL YNSTSFSTEK CYGVSPSKLI
DLCFTSVYAD TFLIRCSEVR QVAPGQTGVI ADYNYKLPDD FTGCVIAWNT AKQDTGSSGN YNYYYRSHRK
TKLKPFERDL SSDECSPDGK PCTPPAFNGV RGENCYFTLS TYDENPNVPV EYQATRVVVL SFELLNANAT
VCGPKLSTQ
>M10
(SEQ ID NO: 36)
RVSPTQEVVR FPQITNLCPF DKVFQATREP SVYAWERTKI SDCVADYTVL YNSTSESTEK CYGVSPSKLI
DLCFTSVYAD TFLIRCSEVR QVAPGQTGVI ADYNYKLPDD FTGCVIAWNT AKQDTGSSGN YNYYYRSHRK
TKLKPFERDL SSDECSPDGK PCTPPAENGV RGENCYFTLS TYDENPNVPV EYQATRVVVL SFELLNAPAT
VCGPKLSTQ

Alignment of these sequences with the SARS2 Reference sequence (EPI_ISL_402119_RBD (CoV_T2_6) (SEQ ID NO:11)) is shown below (with the dots representing no difference in amino acid residue from the reference sequence, and the dashes representing positions where amino acid residues have been inserted in the M9 and M10 sequences):

| | | |
|---|---|---|
| SARS2_RBD | RVQPTESIVRFPNITNLCPFGEVFNATRFASVYAWNRKRISNCVADYSVLYNSASFSTFKCYGV | 64 |
| M7 | ................................................................ | 64 |
| M8 | ............Q...........Q............................T.......... | 64 |
| M9 | ..S..QEV............DK.......P.....E.TK..D.....T.....T.......... | 64 |
| M10 | ..S..QEV............DK..Q....P.....E.TK..D.....T.....T.......... | 64 |
| SARS2_RBD | SPTKLNDLCFTNVYADSFVIRGDEVRQIAPGQTGKIADYNYKLPDDFTGCVIAWNSNNLDSKVG | 128 |
| M7 | ................................................................ | 128 |
| M8 | ................................................................ | 128 |
| M9 | ..S..I.....S....T.L..CS....V......V....................TAKQ.TGSS | 128 |
| M10 | ..S..I.....S....T.L..CS....V......V....................TAKQ.TGSS | 128 |
| SARS2_RBD | GNYNYLYRLFRKSNLKPFERDISTEIYQAGSTPC.....NGVEGFNCYFPLQSYGFQPTNGVGY | 187 |
| M7 | ................................................................ | 187 |
| M8 | ................................................................ | 187 |
| M9 | .....Y..SH..TK.......L.SDECSPDGK..TPPAF...R......T.ST.D.N.NVP.E. | 192 |
| M10 | .....Y..SH..TK.......L.SDECSPDGK..TPPAF...R......T.ST.D.N.NVP.E. | 192 |
| SARS2_RBD | QPYRVVVLSFELLHAPATVCGPKKSTN | 214 |
| M7 | ...............N........... | 214 |
| M8 | ........................... | 214 |
| M9 | .AT..........N.N.......L..Q | 219 |
| M10 | .AT..........N.........L..Q | 219 |

The amino acid differences of the designed sequences from the SARS2 reference sequence are summarised in the table below (with differences from the reference sequence highlighted in bold):

| Circled | SARS2 RBD | | | | | |
|---|---|---|---|---|---|---|
| number of | residue | Reference | | | | |
| FIG. 13 | position | residue | M7 residue | M8 residue | M9 residue | M10 residue |
| | 3 | Q | | | S | S |
| | 6 | E | | | Q | Q |
| | 7 | S | | | E | E |
| | 8 | I | | | V | V |
| 1 | 13 | N | | Q | | Q |
| | 21 | G | | | D | K |
| | 22 | E | | | D | K |
| 2 | 25 | N | | Q | | Q |
| | 30 | A | | | P | P |
| | 36 | N | | | E | E |
| | 38 | K | | | T | K |
| | 39 | R | | | T | K |
| | 42 | N | | | D | D |
| | 48 | S | | | T | T |
| 3 | 54 | A | | T | T | T |
| | 67 | T | | | S | S |
| | 70 | N | | | I | I |
| | 76 | N | | | S | S |
| | 81 | S | | | T | T |
| | 83 | V | | | L | L |
| | 86 | G | | | C | C |
| | 87 | D | | | S | S |
| | 92 | I | | | V | V |
| | 99 | K | | | V | V |
| | 120 | S | | | T | T |
| | 121 | N | | | A | A |
| | 122 | N | | | K | K |
| | 123 | L | | | Q | Q |
| | 125 | S | | | T | T |
| | 126 | K | | | G | G |
| | 127 | V | | | S | S |
| | 128 | G | | | S | S |
| | 134 | L | | | Y | Y |
| | 137 | L | | | S | S |
| | 138 | F | | | H | H |
| | 141 | S | | | T | T |
| | 142 | N | | | K | K |
| | 150 | I | | | L | L |
| | 152 | T | | | S | S |
| | 153 | E | | | D | D |
| | 154 | I | | | E | E |
| | 155 | Y | | | C | C |
| | 156 | Q | | | S | S |
| | 157 | A | | | P | P |
| | 158 | G | | | D | D |
| | 159 | S | | | G | G |
| | 160 | T | | | K | K |
| | | — | | | T | T |
| | | — | | | P | P |
| | | — | | | P | P |
| | | — | | | A | A |
| | | — | | | F | F |
| | 166 | E | | | R | R |
| | 173 | P | | | T | T |
| | 175 | Q | | | S | S |
| | 176 | S | | | T | T |
| | 178 | G | | | D | D |
| | 180 | Q | | | N | N |
| | 182 | T | | | N | N |
| | 183 | N | | | V | V |
| | 184 | G | | | P | P |
| | 186 | E | | | E | E |
| | 189 | P | | | A | A |
| | 190 | Y | | | T | T |
| | 201 | H | | | N | N |
| 4 | 203 | P | N | | N | — |
| | 211 | K | | | L | L |
| | 214 | Q | | | Q | Q |

### EXAMPLE 15

### Nucleotide Sequences of Further Designed S Protein RBD Sequences

Nucleotide sequences encoding the M7, M8, M9, and M10 SARS2 RBD designs discussed in Example 14 are shown below:

>M7
(SEQ ID NO: 37)
cgggtgcagc ccaccgaatc catcgtgcgg ttccccaata tcaccaatct gtgccccttc 60
ggcgaggtgt tcaatgccac cagattcgcc tctgtgtacg cctggaaccg gaagcggatc 120
agcaattgcg tggccgacta ctccgtgctg tacaactccg ccagcttcag caccttcaag 180
tgctacggcg tgtcccctac caagctgaac gacctgtgct tcacaaacgt gtacgccgac 240
agcttcgtga tccggggaga tgaagtgcgg cagattgccc ctggacagac aggcaagatc 300
gccgactaca actacaagct gcccgacgac ttcaccggct gtgtgattgc ctggaacagc 360
aacaacctgg actccaaagt cggcggcaac tacaattacc tgtaccggct gttccggaag 420
tccaatctga agcccttcga gcgggacatc agcaccgaaa tctatcaggc cggcagcacc 480
ccttgcaacg gcgtggaagg cttcaactgc tacttcccac tgcaaagcta cggctttcag 540
cccacaaatg gcgtgggcta ccagccttac agagtggtgg tgctgagctt cgagctgctg 600
catgctaacg ccacagtgtg cggccctaag aaatccacca at 642
>M8
(SEQ ID NO: 38)
cgggtgcagc ccaccgaatc catcgtgcgg ttcccccaga tcaccaatct gtgccccttc 60
ggcgaggtgt tccaggccac cagattcgcc tctgtgtacg cctggaaccg gaagcggatc 120
agcaattgcg tggccgacta ctccgtgctg tacaactcca ccagcttcag caccttcaag 180
tgctacggcg tgtcccctac caagctgaac gacctgtgct tcacaaacgt gtacgccgac 240
agcttcgtga tccggggaga tgaagtgcgg cagattgccc ctggacagac aggcaagatc 300
gccgactaca actacaagct gcccgacgac ttcaccggct gtgtgattgc ctggaacagc 360
aacaacctgg actccaaagt cggcggcaac tacaattacc tgtaccggct gttccggaag 420
tccaatctga agcccttcga gcgggacatc agcaccgaaa tctatcaggc cggcagcacc 480
ccttgcaacg gcgtggaagg cttcaactgc tacttcccac tgcaaagcta cggctttcag 540
cccacaaatg gcgtgggcta ccagccttac agagtggtgg tgctgagctt cgagctgctg 600
catgctcctg ccacagtgtg cggccctaag aaatccacca at 642
>M9
(SEQ ID NO: 39)
cgggtgtccc ctacacaaga ggtcgtgcgg ttccccaata tcaccaatct gtgccccttc 60
gacaaggtgt tcaacgccac cagatttccc agcgtgtacg cctgggagcg caccaagatt 120
tccgattgcg tggccgacta caccgtgctg tataactcca cctccttcag caccttcaag 180
tgctacggcg tgtccccaag caagctgatc gatctgtgct tcacctctgt gtacgccgac 240
accttcctga tccggtgtag cgaagtgcga caggtggcac ctggacagac aggcgtgatc 300
gccgattaca actacaagct gcccgacgac ttcaccggct gtgtgatcgc ctggaatacc 360
gccaagcagg atacaggcag cagcggcaac tacaactact actacagaag ccaccgcaag 420
accaagctga agcctttcga gagggacctg agcagcgacg agtgtagccc tgatggcaag 480
ccttgtacac ctcctgcctt caatggcgtg cggggcttca actgctactt caccctgagc 540
acctacgact tcaaccccaa cgtgcccgtg gaataccagg ccacaagagt ggtggtgctg 600
agcttcgagc tgctgaatgc caacgccaca gtgtgtggcc ctaagctgtc tacccag 657
>M10
(SEQ ID NO: 40)
cgggtgtccc ctacacaaga ggtcgtgcgg ttcccccaga tcaccaatct gtgccccttc 60
gacaaggtgt tccaggccac cagatttccc agcgtgtacg cctgggagcg caccaagatt 120
tccgattgcg tggccgacta caccgtgctg tataactcca cctccttcag caccttcaag 180
tgctacggcg tgtccccaag caagctgatc gatctgtgct tcacctctgt gtacgccgac 240
accttcctga tccggtgtag cgaagtgcga caggtggcac ctggacagac aggcgtgatc 300
gccgattaca actacaagct gcccgacgac ttcaccggct gtgtgatcgc ctggaatacc 360
gccaagcagg atacaggcag cagcggcaac tacaactact actacagaag ccaccgcaag 420
accaagctga agcctttcga gagggacctg agcagcgacg agtgtagccc tgatggcaag 480
ccttgtacac ctcctgcctt caatggcgtg cggggcttca actgctactt caccctgagc 540
acctacgact tcaaccccaa cgtgcccgtg gaataccagg ccacaagagt ggtggtgctg 600
agcttcgagc tgctgaatgc ccctgccaca gtgtgtggcc ctaagctgtc tacccag 657

Differences between these sequences are highlighted in the alignment below (with the dots indicating that the nucleotide residue is the same as the corresponding M7 nucleotide residue):

| | | |
|---|---|---|
| M7 | CGGGTGCAGCCCACCGAATCCATCGTGCGGTTCCCCAATATCACCAATCTGTGCCCCTTCGGCGAGGT | 68 |
| M8 | ....................................C.G............................. | 68 |
| M9 | ......TCC..T..AC..GAGG.......................................A.A.... | 68 |
| M10 | ......TCC..T..AC..GAGG..............C.G......................A.A.... | 68 |
| M7 | GTTCAATGCCACCAGATTCGCCTCTGTGTACGCCTGGAACCGGAAGCGGATCAGCAATTGCGTGGCCG | 136 |
| M8 | ....C.G............................................................. | 136 |
| M9 | ......C...........TC..AGC............G.G..C.CCAA...TTC.G............ | 136 |
| M10 | ....C.G...........TC..AGC............G.G..C.CCAA...TTC.G............ | 136 |
| M7 | ACTACTCCGTGCTGTACAACTCCGCCAGCTTCAGCACCTTCAAGTGCTACGGCGTGTCCCCTACCAAG | 204 |
| M8 | .......................A............................................ | 204 |
| M9 | .....A..........T......A..TC.................................A.G.... | 204 |
| M10 | .....A..........T......A..TC.................................A.G.... | 204 |
| M7 | CTGAACGACCTGTGCTTCACAAACGTGTACGCCGACAGCTTCGTGATCCGGGGAGATGAAGTGCGGCA | 272 |
| M8 | .................................................................... | 272 |
| M9 | ....T...T...........CTCT.............C....C........T.TAGC........A.. | 272 |
| M10 | ....T...T...........CTCT.............C....C........T.TAGC........A.. | 272 |
| M7 | GATTGCCCCTGGACAGACAGGCAAGATCGCCGACTACAACTACAAGCTGCCCGACGACTTCACCGGCT | 340 |
| M8 | .................................................................... | 340 |
| M9 | .G.G..A...............GT.........T.................................. | 340 |
| M10 | .G.G..A...............GT.........T.................................. | 340 |
| M7 | GTGTGATTGCCTGGAACAGCAACAACCTGGACTCCAAAGTCGGCGGCAACTACAATTACCTGTACCGG | 408 |
| M8 | .................................................................... | 408 |
| M9 | .......C........T.C.GC...G.A...TA.AGGCAG.A.............C...TAC...A.A | 408 |
| M10 | .......C........G.C.GC...G.A...TA.AGGCAG.A.............C...TAC...A.A | 408 |
| M7 | CTGTTCCGGAAGTCCAATCTGAAGCCCTTCGAGCGGGACATCAGCACCGAAATCTATCAGGCCGGCAG | 476 |
| M8 | .................................................................... | 476 |
| M9 | AGGCA...C...A....G........T......A.....C.G....G...CGAG.G.AGCC.T.ATG. | 476 |
| M10 | AGGCA...C...A....G........T......A.....C.G....G...CGAG.G.AGCC.T.ATG. | 476 |
| M7 | CACCCCTTGC---------------AACGGCGTGGAAGGCTTCAACTGCTACTTCCCACTGCAAAGCT | 529 |
| M8 | .................................................................... | 529 |
| M9 | ..AG.....TACACCTCCTGCCTTC..T......CGG..................A.C...AGC.C.. | 544 |
| M10 | ..AG.....TACACCTCCTGCCTTC..T......CGG..................A.C...AGC.C.. | 544 |
| M7 | ACGGCTTTCAGCCCACAAATGGCGTGGGCTACCAGCCTTACAGAGTGGTGGTGCTGAGCTTCGAGCTG | 597 |
| M8 | .................................................................... | 597 |
| M9 | ...A...CA..C...ACGTGCC.....AA......G.CACA........................... | 612 |
| M10 | ...A...CA..C...ACGTGCC.....AA......G.CACA........................... | 612 |
| M7 | CTGCATGCTAACGCCACAGTGTGCGGCCCTAAGAAATCCACCAAT | 642 |
| M8 | .........CCT................................. | 642 |
| M9 | ...A....C..............T.........CTG..T...C.G | 657 |
| M10 | ...A....CCCT...........T.........CTG..T...C.G | 657 |

### EXAMPLE 16

### Ability of Different Full-Length S Protein Genes to Induce Antibodies to SARS2 RBD

Mice were immunised with different full-length Coronavirus S protein genes (from SARS-1 and SARS-2), and the sera was collected and tested at different dilutions for binding (by ELISA) to SARS2 RBD. The sera were heat inactivated (HI) to check for non-specific interactions in the ELISA.

The results are shown in FIG. 9.

The binding of the sera to SARS-2 RBD was tested using ELISA. The ELISA protocol is as follows:

### Materials and Reagents:

F96 Nunc Maxisorp flat-bottom plates (Cat #: 44-2404-21, Thermo Scientific)
Plate sealers (Cat #: 676001, Greiner Bio-one)
Shaker (Cat #: 544-11200-00, Heidolph Instruments Titramax 100). 50 mL and 100 mL reservoirs (Cat #4870 Corning and #B3110-100 Argos)
U-bottom dilution plates (Cat #: 650201, Greiner bio-one)
1×PBS (−Ca/−Mg):
   Add 2 PBS tablets (Cat #: 18912-014, Gibco) to 1 L milliQ water
1×PBS (−Ca/−Mg)+0.1% Tween-20 (PBST):
   Add 4 PBS tablets (Cat #: 18912-014, Gibco) and 2 mL Tween-20 (Cat #: P1379-500ML, Sigma Aldrich) to 2 L milliQ water
3% (w/v) non-fat milk in 1×PBST (blocking solution):
   Add 1.5 g of semi-skimmed milk powder (Cat #: 70166-500G, Sigma Aldrich) in 50 ml of PBST
1% (w/v) non-fat milk in 1×PBST (serum dilution solution):
   0.5 g of milk powder (Cat #: 70166-500G, Sigma Aldrich) in 50 ml of PBST
HRP-conjugated secondary antibodies:
   Anti-mouse IgG-horseradish peroxidase (HRP) conjugated secondary antibody (Cat #: 715-035-150, Jackson ImmunoResearch)
   Anti-human IgG/IgM/IgA-horseradish peroxidase (HRP) conjugated secondary antibody (Cat #: 109-035-064, Jackson ImmunoResearch)
1-Step™ Ultra TMB (Cat #34029, Thermo Scientific).
Stop solution of H₂SO₄ (add 28 mL of 1.84 kg/L H₂SO₄ to 472 mL milliQ water).
Serum samples (about 4 μl is needed to run a duplicate, starting at 1:50 dilution with 10-fold serial dilutions; about 5.5 μl is needed to run a duplicated, starting at 1:50 dilution with 2-fold serial dilutions)
Human positive control: strong antibody positive plasma from Covid-19 patient (Cat #20/130, NIBSC)
Human negative control: WHO Reference Anti-EBOV Negative human plasma (Cat #: 15/288, NIBSC)

### Method:

### Day 0

1. Coat ninety-six well Nunc Maxisorp plates with 50 μl (per well) of 1 μg/mL of protein diluted in PBS−/−. Tap the plates gently against the counter to ensure that the liquid has fully coated the bottom of the plate.
2. Seal the plates tightly with plate sealer. Store plates in −4° C. fridge overnight, to a maximum of 4 days. Ensure that the liquid has not evaporated when using.
3. Prepare 3% and 1% non-fat milk, vortex and leave to dissolve on the shaker at 1350 rpm at room temperature. Leave to dissolve for at least one hour. Store in the −4° C. fridge overnight.

### Day 1

4. Prepare the negative and positive controls
   Mouse Negative control: Prepare a pool of all six mice from the PBS-immunized group (usually Group 1) from the corresponding bleed, at a final dilution of 1:50 in 1% non-fat milk in PBST
   Mouse Positive control: Prepare a 1:500 dilution of a known strong positive in 1% non-fat milk in PBST
   Human Negative control: Prepare a 1:50 dilution of the required amount of anti-EBOV plasma in 1% non-fat milk in PBST
   Human Positive control: Prepare a 1:500 dilution of the required amount of 20/130 in 1% non-fat milk in PBST
5. Decant the protein from the 96-well plate and add 100 μl of 3% non-fat milk per well. Incubate for 1 hour at room temperature on the shaker at 200-400 rpm.
6. During the blocking step, prepare serial dilutions of the serum in 1% non-fat milk in PBST using the U-bottom dilution plates.
   For a two-fold serial dilution starting at 1:50-Add 130 μl 1% non-fat milk to the first row with 2.6 μl of serum (in duplicates). Add 65 μl 1% non-fat milk to the remaining rows. Transfer 65 μl for the serial dilutions.
   For a ten-fold serial dilution starting at 1:50-Add 75 μl 1% non-fat milk to the first row with 1.5 μl of serum (in duplicates). Add 63 μl 1% non-fat milk to the remaining rows. Transfer 7 μl for the serial dilutions.
7. After the 1-hour blocking, decant the blocking solution and add 50 μl of the serial dilutions to the corresponding plates. Incubate on the shaker at 200-400 rpm for two hours at room temperature.
8. During the incubation, dilute the HRP-conjugated anti-mouse IgG secondary antibody 1:3000 in PBST. Make up 5 ml of diluted secondary per 96-well plate.
9. After the 2-hour primary antibody incubation, wash the plates three times with 200 μl (per well) of PBST. Tap dry after the last wash. Then add 50 μl (per well) of the diluted secondary antibody. Incubate on the shaker at 200-400 rpm at room temperature for 1 hour.
10. After adding the secondary antibody, take the appropriate volume of TMB and leave it on the counter to come to room temperature. Take 5 mL of TMB per 96-well plate.
11. After the 1-hour secondary antibody incubation, wash the plates three times with 200 μl (per well) of PBST. Tap dry after the last wash.
12. Add 50 μl (per well) of room temperature TMB. Agitate the plate gently. Leave for approximately 2-3 mins. Monitor the plate to ensure that the colour change does not become saturated. Add TMB to a maximum of 5 plates at a time.
13. Add 50 μl (per well) of room temperature stop solution. Agitate the plate gently. Read immediately.
14. Read endpoint optical density at 450 nm.

The following DNA vaccines were used:

### Heat Inactivation (HI)

SARS-1 (DNA encoding full length SARS-1 S protein)
SARS-2 (DNA encoding full length SARS-2 S protein)
DIOS-ancestor (Wuhan Node 1 full length)

### Not HI

SARS-1
SARS-2
DIOS-ancestor

Human sera against SARS-2 and anti-SARS1 spike monoclonal antibody were used as positive controls, and anti-MERS human sera was used as a negative control.

The figure shows that all the full-length S protein genes tested induced a relatively poor or negligible binding response to SARS2 RBD.

### EXAMPLE 17

### Ability of DNA Vaccines Encoding SARS1 and SARS2 Truncated Spike(s) Protein and RBD to Induce Antibodies to SARS1 and SARS2 S Protein

Mice were immunised with different DNA vaccines, and sera collected from the mice was used to test binding by FACS to SARS1 and SARS2 spike protein.

### 1—Reagents and Consumables

HEK293T/17 cells
DMEM with 10% FBS and 1% Pen/strep
OptiMEM
1×PBS
FuGENE-HD
   pEVAC expressing plasmid

### 2—Protocol

### Day 1—Seeding Cells

1. Seed 6-well plates with ˜150,000 cells per well for next day transfection (2 six well plates are enough for one 96 well plate)
2. Incubate overnight at 37° C., 5% CO₂.

### Day 2—Cell Transfection

1. Thaw producer cell plasmid DNA and pre-warm DMEM and OptiMEM to 37° C.
2. Prepare DNA mix in 600 μl OptiMEM (amount per plate; see table 1) in a labelled 1.5 ml tube
3. Incubate DNA mix for 5 minutes at room temperature
4. Add 9 μl of FuGENE-HD transfection reagent per 3 μg DNA in the transfection complex (see table below)
5. Incubate at room temperature for 20 minutes; mix by gently flicking the tube.
6. During incubation, remove depleted media from each well of the 6 well plate and replace with 2 ml DMEM per well.
7. After incubation, add the transfection complex to cells in a dropwise manner, and swirl to ensure even distribution.
8. Return cells to tissue culture incubator (37° C., 5% CO₂)

| Transfection | Glycoprotein | OptiMEM | FuGENE HD |
|---|---|---|---|
| complex | plasmid (ng) | (μl) | (μl) |
| pEVAC SARS1 | 3000 | 600 | 9.0 |
| pEVAC SARS2 | 3000 | 600 | 9.0 |

### Day 3—Antibody/Serum Dilution

1. Perform 1:2 serial dilution of serum or antibodies in cold PBS 1% FBS (e.g. 6 μl of serum in 300 μl of buffer, aliquot 150 μl for a duplicate. (6-well U-plate is preferred)
2. Human serum or IgG isotype controls must be included in the experimental plan

### Day 4—Flow Cytometry

3. Remove media and collect cells in a falcon
4. Centrifuge 5′ at 300×g
5. Resuspend cell pellet in 10 ml PBS (per plate)
6. Aliquot 100 μl of cell suspension per well in a 96 well plate V-bottom, using P100 multichannel and reservoir.
7. Centrifuge the plate 2′ at 300×g (R2 rotor in 227)
8. Flick out the plate in the sink
9. By using a multichannel, transfer 75 μl of diluted serum or antibodies from dilution plate to the FCAS plate and resuspend cells
10. Incubate RT 40′
11. Wash plate by adding 100 μl of PBS
12. Centrifuge the plate 2′ at 300×g
13. Flick out the plate in the sink
14. Wash plate by adding 180 μl of PBS and resuspend cell pellet
15. Flick out the plate
16. Add 60 μl/well of secondary antibody (20 μl/ml) and resuspend cells
17. Incubate RT 40′
18. Wash plate by adding 100 μl of PBS
19. Centrifuge the plate 2′ at 300×g
20. Flick out the plate in the sink
21. Wash plate by adding 180 μl of PBS and resuspend cells
22. Flick out the plate
23. Resuspend cells in 200 μl of PBS
   The DNA Vaccines Used were:

| | |
|---|---|
| COV_S_T2_2 | AY274119_tr (CoV_T2_2): nucleic |
| | acid encoding truncated S-protein |
| | (SEQ ID NO: 4) |
| COV_S_T2_3 | EPI_ISL_402119_tr (CoV_T2_3): nucleic |
| | acid encoding truncated S-protein |
| | (SEQ ID NO: 10) |
| COV_S_T2_5 | AY274119_RBD (CoV_T2_5): nucleic |
| | acid encoding RBD (SEQ ID NO: 6) |
| COV_S_T2_6 | EPI_ISL_402119_RBD (CoV_T2_6): nucleic |
| | acid encoding RBD (SEQ ID NO: 12) |
| COV_S_T2_7 | Wuhan_Node1_RBD (CoV_T2_7): nucleic |
| | acid encoding RBD (SEQ ID NO: 18) |
| COV_S_T2_8 | “SARS_2 RBD_mut1” (the M7 construct, |
| | SEQ ID NO: 37) |
| COV_S_T2_10 | “SARS_an RBD_mut1” (the M9 construct, |
| | SEQ ID NO: 39) |

Binding of the sera obtained following the immunisations to SARS1 spike protein and SARS2 spike protein, at different dilutions, was assessed by FACS. The results are shown in FIG. 10.

The results show that the sera collected following immunisation with DNA encoding truncated spike protein and the RBD domains binds to the respective SARS protein. The M7 construct induced sera with better binding than the corresponding wild type SARS2 RBD.

### EXAMPLE 18

### Ability of DNA Vaccines Encoding Wild-Type SARS1 or SARS2 Spike Protein (Full-Length, Truncated, or RBD) to Induce a Neutralisation Response to SARS1 and SARS2 Pseudotypes

Mice were immunised with DNA vaccine encoding wild-type full-length SARS1 or SARS2 spike protein, DNA vaccine encoding wild-type truncated SARS1 or SARS2 spike protein, DNA vaccine encoding wild-type SARS1 or SARS2 spike RBD protein, or wild-type SARS1 or SARS2 RBD protein. Sera collected from the immunised mice were tested at different dilutions for their ability to neutralise SARS1 or SARS2 pseudotypes.

The vaccines used were:
DNA encoding full-length SARS1 or SARS2 spike protein;
DNA encoding truncated SARS1 or SARS2 spike protein;
DNA encoding SARS1 or SARS2 spike RBD; and
SARS1 or SARS2 RBD protein.

PBS was used as a negative control, and 20/130 (a National Institute for Biological Standards and Control (NIBSC) standard) and serum from patient 4 (a COVID-19 patient with strongly neutralising antibodies) were used as positive controls.

The results are shown in FIG. 11.

The results show that mice immunised with the SARS1 immunogens (DNA or protein) induce antibodies which neutralise SARS1 pseudotypes. However, the only SARS2 immunogen which induces SARS2 pseudotype neutralising antibodies is the DNA encoding SARS2 RBD.

### EXAMPLE 19

### Ability of SARS1 and SARS2 RBD Protein Vaccines to Induce Antibodies to SARS2 RBD

Mice were immunised with different protein vaccines. The sera were collected and tested for binding to SARS2 RBD at different dilutions.

The vaccines used were:
P-RBD-CoV1 (wild-type SARS1 RBD protein)
P-RBD-CoV2 (wild-type SARS2 RBD protein)
P-S_Stab_CoV2 (full-length spike protein stabilised by two proline mutations and removal of transmembrane region)

The results are shown in FIG. 12.

The results show that all of the protein vaccines tested induced SARS2 RBD-binding antibodies, including the SARS1 RBD (P-RBD-CoV1).

### EXAMPLE 20

### Ability of Different S Protein RBD DNA Vaccines to Induce Antibodies to SARS2 RBD

Mice were immunised with different S protein (truncated or RBD) DNA vaccines, then sera was collected and tested for binding to SARS2 RBD by ELISA (using the protocol described in Example 16).

The Vaccines Used were:
Ancestor RBD
Conv373 (positive control-sera from a Covid positive patient; data not shown)
Human_s (negative control, pre-Covid serum from Sigma)
SARS_1 RBD
SARS_1 trunc
SARS_2 RBD
SARS_2 RBD_mut1 (M7)
SARS_2 trunc
SARS_anc RBD_mut1 (M9)

The results are shown in FIG. 14.

The results show that the M7 SARS2 RBD DNA vaccine induced an immune response with stronger binding to SARS2 RBD than wild-type SARS2 RBD DNA in the early bleed.

### EXAMPLE 21

Inhibition of RBD-ACE2 Interaction by Sera Collected Following Immunisation with M7 and Wild-Type SARS2 RBD DNA Vaccines

A competition assay was used to show to what extent mouse sera, after immunisation of mice with M7 and wild-type RBD DNA vaccines, prevents binding of SARS2 pseudotypes to ACE2 receptors, using sera collected 2 and 8 weeks after immunisation.

The DNA vaccines used were:
D-RBD-CoV2 (DNA encoding wild-type SARS2 RBD);
D-RBD-M7_CoV2 (DNA encoding M7 SARS2 RBD).
D-RBD-TM_CoV2 (DNA encoding wild type RBD with a transmembrane domain, so that it remains tethered to the cell membrane rather than released as soluble protein like other RBD constructs)

The results are shown in FIG. 15.

The results presented in the left hand figure (a) (week 2) show that sera collected 2 weeks after immunisation with DNA encoding wild-type RBD and tethered wild-type RBD has no effect on binding of SARS2 pseudotypes to ACE2 receptors, but the sera collected 2 weeks after immunisation with DNA encoding M7 RBD does inhibit binding of SARS2 pseudotypes to ACE2 receptors.

The results presented in the right hand figure (b) (week 8) show that sera collected 8 weeks after immunisation with DNA encoding wild-type RBD and M7 RBD both show strong neutralisation.

It was concluded from these results that the DNA vaccine encoding wild-type RBD and M7 RBD elicit a neutralising immune response 8 weeks after immunisation, but that DNA vaccine encoding M7 SARS2 RBD elicits a neutralising immune response more rapidly than DNA vaccine encoding wild-type SARS2 RBD.

### Methods:

The competition assay was carried out using the GenScript SARS-CoV-2 Surrogate Virus Neutralization Test (SVNT) Kit, according to the manufacturer's protocol. The kit can detect circulating neutralizing antibodies against SARS-CoV-2 that block the interaction between the receptor binding domain of the viral spike glycoprotein (RBD) with the ACE2 cell surface receptor. The assay detects any antibodies in serum and plasma that neutralize the RBD-ACE2 interaction. The test is both species and isotype independent.

First, the samples and controls are pre-incubated with the HRP-RBD to allow the binding of the circulating neutralization antibodies to HRP-RBD. The mixture is then added to the capture plate which is pre-coated with the hACE2 protein. The unbound HRP-RBD as well as any HRP-RBD bound to non-neutralizing antibody will be captured on the plate, while the circulating neutralization antibodies-HRP-RBD complexes remain in the supernatant and get removed during washing. After washing steps, TMB solution is added, making the colour blue. By adding Stop Solution, the reaction is quenched and the colour turns yellow. This final solution can be read at 450 nm in a microtiter plate reader. The absorbance of the sample is inversely dependent on the titre of the anti-SARS-CoV-2 neutralizing antibodies.

### EXAMPLE 22

### Neutralisation of SARS2 Pseudotype Induced by M7 and Wild-Type SARS2 RBD DNA Vaccines

Mice were immunised with different RBD DNA vaccines listed below, then sera was collected and tested for SARS2 pseudotype neutralisation. Two studies were carried out (COV002.1 and COV002.2).

The DNA Vaccines Used were:
Ancestor RBD (DNA encoding ancestor RBD);
SARS_1 RBD (DNA encoding wild-type SARS1 RBD);
SARS_1 trunc (DNA encoding wild-type SARS1 truncated S protein);
SARS_2 RBD (DNA encoding wild-type SARS2 RBD)
SARS_2 RBD_mut1 (M7) (DNA encoding M7 SARS2 RBD)
SARS_2 trunc (DNA encoding wild-type SARS2 truncated S protein)
SARS_anc RBD_mut1 (M9) (DNA encoding M9 SARS ancestor RBD)

The results are shown in FIGS. 16 and 17.

The results from study COV002.1 and COV002.2 are shown in FIG. 16(a) (bleed at week 2 from the immunised mice), and the results from study COV002.1 and COV002.2 are shown in FIGS. 16(b) (bleed at week 3 from the immunised mice), and 16(c) (bleed at week 4 from the immunised mice).

FIG. 17 shows SARS2 pseudotype neutralisation IC₅₀ values for sera collected from the mice immunised with wild-type SARS2 RBD DNA vaccine, and M7 SARS2 RBD DNA vaccine. The dots in FIG. 17 show IC₅₀ values for individual mice, and the horizontal cross bars show the estimate based on all mice with 95% confidence intervals. The results shown in FIG. 17(a) are from study COV002.1 and COV002.2. The results shown in FIG. 17(b) are from study COV002.2.

The results in FIGS. 16 and 17 show that the M7 SARS2 RBD DNA vaccine induces a more neutralising response than the wild-type SARS2 RBD DNA vaccine in sera collected from bleeds at weeks 1 and 2, but that by later bleeds there appears to be little difference between the two vaccines.

### EXAMPLE 23

Supernatant of Cells Expressing M7 SARS2 RBD Competes with Other ACE2 Binding Viruses for ACE2 Cell Entry

Supernatant of cells was used to compete with one of three coronavirus pseudotypes (NL63, SARS1, SARS2) for ACE2 receptors. The supernatant was either from cells expressing M7 or from cells transfected with the empty pEVAC. The results are shown in FIG. 18.

The results show that the M7 supernatant competes effectively with the three ACE2 binding viruses, although possibly to a lesser extent with SARS1.

### EXAMPLE 24

### M7 SARS2 RBD DNA Vaccine Induces T Cell Responses

An enzyme-linked immunospot (ELISPOT) assay against an RBD peptide pool was used to determine T cell responses induced by the M7 SARS2 RBD DNA vaccine (compared with PBS as a negative control). The results are shown in FIG. 19. The results show that T cell responses were induced by the M7 DNA vaccine that were reactive against peptides of the RBD peptide pool. The medium is used as the negative control.

The ELISPOT assay is a highly sensitive immunoassay that measures the frequency of cytokine-secreting cells (in this case, murine T cells secreting IFN-γ) at the single-cell level. In this assay, cells are cultured on a surface coated with a specific capture antibody in the presence or absence of stimuli. Proteins, such as cytokines, that are secreted by the cells will be captured by the specific antibodies on the surface. After an appropriate incubation time, cells are removed and the secreted molecule is detected using a detection antibody in a similar procedure to that employed by the enzyme-linked immunoassay (ELISA). The detection antibody is either biotinylated and followed by a streptavidin-enzyme conjugate or the antibody is directly conjugated to an enzyme. By using a substrate with a precipitating rather than a soluble product, the end result is visible spots on the surface. Each spot corresponds to an individual cytokine-secreting cell.

The ELISPOT assay was carried out according to the manufacturer's protocol (Cellular Technology Limited, CTL) repeated below:

### Murine IFN-γ Single-Color Enzymatic ELISPOT Assay:

PROCEDURE (If using precoated plates, start at Day 1)

### Day 0—Sterile Conditions

Prepare Murine IFN-γ Capture Solution (see Solutions).
Pipette 80 μl/well Murine IFN-γ Capture Solution. Seal plate with parafilm and incubate at 4° C. overnight. (Prewetting of plates with ethanol is not required but in some instances where a large response is expected, the assay can benefit from removing the underdrain, adding 15 μl of 70% ethanol/well for less than one minute, washing three times with 150 μl of PBS/well, replacing the underdrain, and immediately [before plate dries], add the Capture Solution. If using strip plates, there is no underdrain to remove before prewetting. As an alternative, one can purchase CTL precoated plates.) Note: Activation of the membrane with ethanol is instantaneous and can be seen visually as a graying of the membrane. Ethanol should be washed off as quickly as possible following activation.

### Day 1—Sterile Conditions

Prepare CTL-Test™ Medium (see Solutions).
Prepare antigen/mitogen solutions at two times final concentration in CTL-Test™ Medium.
Decant plate containing Capture Solution from Day 0 and wash one time with 150 μl PBS.
Plate antigen/mitogen solutions, 100 μl/well. Ensure the pH and temperature are ideal for cells by placing the plate containing antigens into a 37° C. incubator for 10-20 minutes before plating cells.
Adjust cells to desired concentration in CTL-Test™ Medium, e.g.: 3 million/ml corresponding to 300,000 cells/well (cell numbers can be adjusted according to expected spot counts since 100,000-800,000 cells/well will provide linear results). Keep cells at 37° C. in humidified incubator, 9% CO₂ while processing cells and until plating.
Plate cells 100 μl/well using large orifice tips. Once completed, gently tap the sides of the plate and immediately place into a 37° C. humidified incubator, 9% CO₂.
Incubate for 24 hours. Do not stack plates. Avoid shaking plates by carefully opening and closing incubator door.

Do not touch plates during incubation.

### Day 2

Prepare Buffer Solutions: PBS, distilled water and Tween-PBS (see Wash Buffers).
Prepare Anti-murine IFN-γ Detection Solution (see Solutions).
Wash plate two times with PBS and then two times with 0.05% Tween-PBS, 200 μl/well each time.
Add 80 μl/well Anti-murine IFN-γ Detection Solution. Incubate at room temperature, two hours.
Prepare Tertiary Solution (see Solutions).
Wash plate three times with 0.05% Tween-PBS, 200 μl/well.
Add 80 μl/well of Tertiary Solution. Incubate at room temperature, 30 minutes.
During incubation, prepare Blue Developer Solution (see Solutions).
Wash plate two times with 0.05% Tween-PBS, and then two times with distilled water, 200 μl/well each time.
Add Blue Developer Solution, 80 μl/well. Incubate at room temperature, 15 minutes.
Stop reaction by gently rinsing membrane with tap water, decant, and repeat three times.
Remove protective underdrain from the plate and rinse back of plate with tap water.
Air-dry plate for two hours in running laminar flow hood or for 24 hours face down on paper towels on bench top.
Scan and count plate. (CTL has scanning and analysis services available and offers a trial version of ImmunoSpot® Software with the purchase of any kit. Email kitscanningservices@immunospot.com for more info.)

### Solutions

All solutions should be freshly-made prior to use. It is important to quick-spin the vials before use to ensure content volumes.
70% Ethanol (if prewetting—not included): Dilute 190-200 proof ethanol. For 10 ml, add 7 ml of ethanol to 3 ml of distilled water.
CTL-Test™ Medium: Prepare medium by adding 1% fresh L-glutamine. The amount of medium needed will depend on variables such as cell yield and number of samples tested but will be no less than 20 ml for one full plate.
Capture Solution: Dilute Murine IFN-γ Capture Antibody in Diluent A. For one plate, add 60 μl of Murine IFN-γ Capture Antibody to 10 ml of Diluent A.
Detection Solution: Dilute Anti-murine IFN-γ (Biotin) Detection Antibody in Diluent B. For one plate, add 10 μl of Anti-murine IFN-γ (Biotin) Detection Antibody to 10 ml of Diluent B.
Tertiary Solution: Dilute Strep-AP Solution in Diluent C,1:1000. For one plate, add 10 μl of Strep-AP to 10 ml of Diluent C.
Blue Developer Solution: Add the Substrate Solutions in sequential steps to 10 ml of Diluent Blue.

For one plate:

Step 1—Add 160 μl of S1 to 10 ml of Diluent Blue. Mix well!

Step 2—Add 160 μl of S2. Mix well!

Step 3—Add 92 μl of S3. Mix well!

It is recommended to make the Blue Developer Solution within ten minutes of use and to keep it protected from direct light.

Wash Buffers (not included)

For each plate prepare:
0.05% Tween-PBS: 100 μl Tween-20 in 200 ml PBS
PBS, sterile, 100 ml
Distilled water, 100 ml

### Cryopreservation of Mouse Splenocytes

This was carried out according to the protocol of CELLULAR TECHNOLOGY LIMITED, repeated below:

Cell permeability, reagent toxicity, and cooling rates must be considered for each cell type when freezing. The osmotic pressure caused by DMSO (more than DMSO's intrinsic toxicity) is one of the primary factors that need to be controlled for successful freezing and thawing of splenocytes. To maintain the metabolic activity of the cells and their membrane lipid fluidity (so they can compensate for the osmotic pressure), all reagents should be at room temperature (preferably at 37° C.).

### Preparation:

1. Mix CTL-Cryo™ A with CTL-Cryo™ B in an 80% to 20% (v/v) ratio (4+1) by slowly adding CTL-Cryo™ B into CTL-Cryo™ A.

(CTL-Cryo™ B contains DMSO as a component. Please refer to MSDS, included.)

2. Warm the resulting CTL-Cryo™ A-B Mix and CTL-Cryo™ C in a 37° C. CO2 incubator. (It is advised to start with this step while counting cells).

3. Each cryotube should contain approximately 10×10⁶ cells (10-15 million). Freezing more cells per tube may lead to cell loss.

### After Washing:

1. After counting, centrifuge the cell suspension at room temperature at 330 g for 10 minutes with rapid acceleration and brake on high.

2. Decant supernatant and mix cells gently by tapping the tube with your finger. Do not use a pipette and avoid foam formation!

3. Slowly, over a time period of ˜2 minutes, add an equal volume of warm CTL-Cryo™ A-B Mix to the CTL-Cryo™ C containing the splenocytes. (Add CTL-Cryo™ A-B mix drop-by-drop while gently whirling the tube to ensure complete mixing of the two solutions.

4. Aliquot the resulting CTL-Cryo™ A-B-C suspension containing the splenocytes into pre-labeled 1.8 ml cryovials, 1 ml into each vial.

Pipette gently and slowly to minimize shear forces; do not attempt additional mixing with the pipette. The cells can remain in the completed CTL-Cryo™ A-B-C medium for 10-20 minutes without loss of viability or function.

5. Place cryovials into a room temperature Nalgene® cryofreezing container (Mr. Frosty™) filled with propanol and transfer into a −80° C. freezer for a minimum of 12 hours. Do not open the freezer during this time period. Use a dedicated −80° C. freezer in order to prevent shaking the samples and fluctuation of the freezer's temperature due to opening and closing of the freezer door.

6. After a minimum of 12 hours and no more than 48 hours, transfer the cryovials into vapor/liquid nitrogen tanks for storage.

### EXAMPLE 25

Further Designed E Protein Sequences (with Abrogated Ion Channel Activity)

SARS-CoV envelope (E) gene encodes a 76-amino acid transmembrane protein with ion channel (IC) activity, an important function in virus-host interaction. Infection of mice with viruses lacking or displaying E protein IC activity revealed that activation of the inflammasome pathway, and the exacerbated inflammatory response induced by SARS-CoV, was decreased in infections by ion channel-deficient viruses (Nieto-Torres et al., 2014, Severe Acute Respiratory Syndrome Coronavirus Envelope Protein Ion Channel Activity Promotes Virus Fitness and Pathogenesis. PLOS Pathog 10(5): e1004077).

We have made new E protein designs Cov_E_T2_3, COV_E_T2_4 and CoV_E_T2_5, which correspond to SARS2, CoV_E_T2_1 and CoV_E_T2_2 (see Example 10), respectively. The new designs have a point mutation, N15A, which abrogates the ion channel activity, but does not influence the stability of the structure. Nieto-Torres et al., supra, discusses this mutation as well as the toxicity and inflammatory action of SARS E on the host cell.

The amino acid sequences of the new E protein designs are shown below:

>COV_E_T2_3 (SARS2_mutant)
(SEQ ID NO: 42)
MYSFVSEETG TLIVASVLLF LAFVVFLLVT LAILTALRLC
AYCCNIVNVS LVKPSFYVYS RVKNLNSSR- VPDLLV
>COV_E_T2_4 (Env1_mutant)
(SEQ ID NO: 43)
MYSFVSEETG TLIVASVLLF LAFVVFLLVT LAILTALRLC
AYCCNIVNVS LVKPTFYVYS RVKNLNSSQG VPDLLV
>COV_E_T2_5 (Env2_mutant)
(SEQ ID NO: 44)
MYSFVSEETG TLIVASVLLF LAFVVFLLVT LAILTALRLC
AYCCNIVNVS LVKPTFYVYS RVKNLNSSR- VPDLLV

Alignment of the E protein designs with SARS2 E protein reference sequence is shown below:

| | | |
|---|---|---|
| SARS2_ COV_E_T2_1 COV_E_T2_2 COV_E_T2_3 COV_E_T2_4 COV_E_T2_5 | 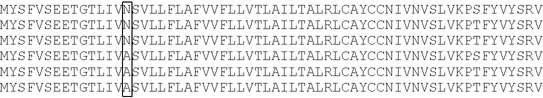 | 62 62 62 62 62 62 |
| SARS2 | 63 KNLNSSR-VPDLLV | 75 |
| COV_E_T2_1 | 63 KNLNSSQGVPDLLV | 76 |
| COV_E_T2_2 | 63 KNLNSSR-VPDLLV | 75 |
| COV_E_T2_3 | 63 KNLNSSR-VPDLLV | 75 |
| COV_E_T2_4 | 63 KNLNSSQGVPDLLV | 76 |
| COV_E_T2_5 | 63 KNLNSSR-VPDLLV | 75 |

The amino acid differences of the designed sequences from the SARS2 reference sequence are shown in the table below (with differences from the reference sequence highlighted in bold):

| | SARS2 | | | | | |
|---|---|---|---|---|---|---|
| SARS2 E | Reference | | | | | |
| protein | Amino | COV_E_T2_1 | COV_E_T2_2 | COV_E_T2_3 | COV_E_T2_4 | COV_E_T2_5 |
| residue | acid | Amino acid | Amino acid | Amino acid | Amino acid | Amino acid |
| position | residue | residue | residue | residue | residue | residue |
| 15 | N | N | N | A | A | A |
| 55 | S | T | T | S | T | T |
| 69 | R | Q | R | R | Q | R |
| 70 | — | G | — | — | G | — |

### EXAMPLE 26

### Nucleoprotein (N) Protein Vaccine Sequences

We have made new N protein designs, COV_N_T2_1 and COV_N_T2_2. The amino acid sequences of these designs is shown below. Sequence COV_N_T2_2 was designed using a methodology and algorithm which selected predicted epitopes to include based on their conservation across the sarbecoviruses (whilst minimising redundancy), the frequency and number of MHC alleles the epitope is restricted by the predicted epitope quality, and a handful of user specified weightings.

>YP 009724397.2/1-419 nucleocapsid phosphoprotein [SARS-COV-2]
(reference sequence)
(SEQ ID NO: 45)
MSDNGPQ-NQ RNAPRITFGG PSDSTGSNQN GERSGARSKQ RRPQGLPNNT ASWFTALTQH GKEDLKFPRG
QGVPINTNSS PDDQIGYYRR ATRRIRGGDG KMKDLSPRWY FYYLGTGPEA GLPYGANKDG IIWVATEGAL
NTPKDHIGTR NPANNAAIVL QLPQGTTLPK GFYAEGSRGG SQASSRSSSR SRNSSRNSTP GSSRGTSPAR
MAGNGGDAAL ALLLLDRLNQ LESKMSGKGQ QQQGQTVTKK SAAEASKKPR QKRTATKAYN VTQAFGRRGP
EQTQGNFGDQ ELIRQGTDYK HWPQIAQFAP SASAFFGMSR IGMEVTPSGT WLTYTGAIKL DDKDPNEKDQ
VILLNKHIDA YKTFPPTEPK KDKKKKADET QALPQRQKKQ QTVTLLPAAD LDDFSKQLQQ SMSSA--DST
QA
>COV_N_T2_1/1-418 Node1b 321-323 deleted
(SEQ ID NO: 46)
MSDNGPQ-NQ RSAPRITFGG PSDSTDNNQN GERSGARPKQ RRPQGLPNNT ASWFTALTQH GKEDLRFPRG
QGVPINTNSG KDDQIGYYRR ATRRVRGGDG KMKELSPRWY FYYLGTGPEA ALPYGANKEG IVWVATEGAL
NTPKDHIGTR NPNNNAAIVL QLPQGTTLPK GFYAEGSRGG SQASSRSSSR SRGNSRNSTP GSSRGTSPAR
MASGGGDTAL ALLLLDRLNQ LESKVSGKGQ QQQGQTVTKK SAAEASKKPR QKRTATKQYN VTQAFGRRGP
EQTQGNFGDQ ELIRQGTDYK HWPQIAQFAP SASAFFGMSR ---EVTPSGT WLTYHGAIKL DDKDPQFKDN
VILLNKHIDA YKTEPPTEPK KDKKKKADEA QPLPQRQKKQ PTVTLLPAAD LDDESKQLQN SMSGASADST
QA
>COV N T2 2/1-417 epitope optimised 321-323 deleted
(SEQ ID NO: 47)
MTDNGQQ-GP RNAPRITF-G VSDNEDNNQD GGRSGARPKQ RRPQGLPNNT ASWFTALTQH GKEDLRFPRG
QGVPINTNSS PDDQIGYYRR ATRRIRGGDG KMKDLSPRWY FYYLGTGPEA ALPYGANKEG IVWVATEGAL
NTPKDHIGTR NPNNNAAIVL QLPQGTTLPK GFYAEGSRGG SQASSRSSSR SRNSSRNSTP GSSRGTSPAR
NLQAGGDTAL ALLLLDRINQ LESKMSGKGQ QQQGQTVTKK SAAEASKKPR QKRTATKQYN VTQAFGRRGP
EQTQGNFGDQ ELIRQGTDYK QWPQIAQFAP SASAFFGMSR ---EVTPSGT WLTYTGAIKL DDKDPQFKDN
VILLNKHIDA YKTFPPTEPK KDKKKKADEA QPLPQRQKKQ QTVTLLPAAD LDDESRQLQN SMSGASADST
QA

Alignment of the N protein designs with SARS2 N protein reference sequence is shown below.

| | | |
|---|---|---|
| YP_009724397 | MSDNGPQNQRNAPRITFGGPSDSTGSNQNGERSGARSKQRRPQGLPNNTASWFTALTQHGKEDLKFPRGQGYPINTNSSPDDQIGYYRRATRRIRGGDGKMKDLSPRW | 108 |
| COV_N_T2_1 | MSDNGPQNQRSAPRITFGGPSDSTQNNQNGERSGARPKQRRPQGLPNNTASWFTALTQHGKEDLRFPRGQGVPINTNSGKDDQIGYYRRATRRVRGGDGKMKELSPRW | 108 |
| COV_N_T2_2 | MTDNGQQGPRNAPRITF-GVSDNFDNNQDGGRSGARPKQRRPQGLPNNTASWFTALTQHGKEDLRFPRGQGVPINTNSSPDDQIGYYRRATRRIRGGDGKMKDLSPRW | 107 |
| YP_009724397 | YFYYLGTGPEAGLPYGANKDGIIWVATEGALNTPKDHIGTRNPANNAAIVLQLPQGTTLPK-GFYAEGERGGSQASSRSSSRSRNSSRNSTPGSSRGTSPARMAGNGG | 215 |
| COV_N_T2_1 | YFYYLGTGPEAALPYGANKEGIVWVATEGALNTPKDHIGTRNPNNNAAIVLQLPQGTTLPF-GFYAEGSRGGSQASSRSSSRSRGNSRNSTPGSSRGTSPARMASGGG | 215 |
| COV_N_T2_2 | YFYYLGTGPEAALPYGANKEGIVWVATEGALNTPKDHIGTPNPNNNAAIVLQLPQGTTLPK-GFYAEGSRGGSDASSRSSSRSRNSSRNSTPGSBRGTSPARNLGAGG | 214 |
| YP_009724397 | DAALALLLLDRLNQLESKMSGKGQQQQGQTYTKKSAAEASKKPRQKRTATKAYNVTQAFGRRGPEQTQGNFGDQELIRQGTDYKHWPQIAQFAPSASAFFGMSRIGME | 323 |
| COV_N_T2_1 | DTALALLLLDRLNQLESKVSGKGQQQQGQTYTKKSAAEASKKPRQKRTATKQYNVTQAFGRRGPEQTQGNFGDQELIRQGTDYKHWPQIAQFAPSASAFFGMSR---E | 320 |
| COV_N_T2_2 | DTALALLLLDRLNQLESKMSGKGQQQQGQTVTKKSAAEASKKPRQKRTATKQYNYTQAFGRPGPEQTQGNFGDQELIRQGTDYKQWPQIAQFAPSASAFFGMSR---E | 319 |
| YP_009724397 | VTPSGTWLTYTGAIKLDDKDPNFKDQVILLNKHIDAYKTFPPTEPKKDKKKKADETQALPQRQKKQQTVTLLPAAD-LDDFSKQLQQSMSSA--DSTQA | 419 |
| COV_N_T2_1 | VTPSGTWLTYHGAIRLDDKDFQFKDNVILLNRHIDAYKTFPPTEPKKDKKKKADEAQPLPQRQKKQPTVTLLPAAD-LDDFSKQLQNSMSGASADSTQA | 418 |
| COV_N_T2_2 | VTPSGTWLTYTGAIKLDDKDPQFKDNYILLNKHIDAYKTFPPTEPKKDKKKKADEAQPLPQRQKKQQTVTLLPAAD-LDDFSRQLQNSMSGASADSTQA | 417 |

The amino acid differences of the designed sequences from the SARS2 reference sequence are shown in the table below (with differences from the reference sequence highlighted in bold, and differences that are common to all the designed sequences underlined):

| SARS2 N | | N_T2_1 | N_T2_2 |
|---|---|---|---|
| protein residue | SARS2 Reference | amino acid | amino acid |
| position (SEQ | amino acid residue | residue (SEQ | residue (SEQ |
| ID NO: 45) | (SEQ ID NO: 45) | ID NO: 46) | ID NO: 47) |
| 2 | S | S | T |
| 6 | P | P | Q |
| 8 | N | N | G |
| 9 | Q | Q | P |
| 11 | N | S | N |
| 18 | G | G | — |
| 20 | P | P | V |
| 23 | S | S | N |
| 24 | T | T | F |
| 25 | G | D | D |
| 26 | S | N | N |
| 29 | N | N | D |
| 31 | E | E | G |
| 37 | S | P | P |
| 65 | K | R | R |
| 79 | S | G | S |
| 80 | P | K | P |
| 94 | I | V | I |
| 103 | D | E | D |
| 120 | G | A | A |
| 128 | D | E | E |
| 131 | I | V | V |
| 152 | A | N | N |
| 192 | N | G | N |
| 193 | S | N | S |
| 211 | A | A | L |
| 212 | G | S | Q |
| 213 | N | G | A |
| 217 | A | T | T |
| 234 | M | V | M |
| 267 | A | Q | Q |
| 300 | H | H | Q |
| 320 | I | — | — |
| 321 | G | — | — |
| 322 | M | — | — |
| 334 | T | H | T |
| 345 | N | Q | Q |
| 349 | Q | N | N |
| 379 | T | A | A |
| 390 | Q | P | Q |
| 406 | K | K | R |
| 409 | Q | N | N |
| 413 | S | G | G |
| 415 | — | S | S |
| 416 | — | A | A |

Positions 415 and 416 are italicised as they are not residues of the reference sequences, but include insertions in the N_T2_1 and N_T2_2 sequences.

### EXAMPLE 27

### Membrane (M) Protein Vaccine Sequences

We have made further new M protein designs. In these designs, we have deleted the 1st and the 2nd transmembrane region of the membrane protein to abrogate its interaction with the S protein:
The string construct with S, M and E was showing higher order aggregates.
Abrogation of interaction between S and M—can reduce aggregation.
M-del constructs (Cov_M_T2_(3-5)) designed to abrogate the interaction with S.

FIG. 20 shows an illustration of the M protein. Interaction between the M, E and N proteins is important for viral assembly. The M protein also binds to the nucleocapsid, and this interaction promotes the completion of virion assembly. These interactions have been mapped to the C-terminus of the endo-domain of the M protein, and the C-terminal domain of the N-protein. In FIG. 20, * denotes identification of immunodominant epitopes on the membrane protein of the Severe Acute Respiratory Syndrome-Associated Coronavirus, and ** denotes mapping of the Coronavirus membrane protein domains involved in interaction with the Spike protein.

The amino acid sequences of the new M protein designs are given below:

>COV_M_T2_3
(SEQ ID NO: 48)
MADSNGTITV EELKKLLEQI TGGIAIAMAC LVGLMWLSYF IASFRLFART RSMWSENPET NILLNVPLHG
TILTRPLLES ELVIGAVILR GHLRIAGHHL GRCDIKDLPK EITVATSRTL SYYKLGASQR VAGDSGFAAY
SRYRIGNGKL NTDHSSSSDN IALLVQ
>COV_M_T2_4
(SEQ ID NO: 49)
MADNGTITVE ELKQLLEQVT GGIAIAMACI VGLMWLSYFV ASFRLFARTR SMWSENPETN ILLNVPLRGT
ILTRPLMESE LVIGAVIIRG HLRMAGHSLG RCDIKDLPKE ITVATSRTLS YYKLGASQRV GTDSGFAAYN
RYRIGNGKLN TDHAGSNDNI ALLVQ
>COV_M_T2_5
(SEQ ID NO: 50)
MADSNGTITV EELKKLLEQV TGGIAIAMAC IVGLMWLSYF VASFRLFART RSMWSENPET NILLNVPLRG
SIITRPLMES ELVIGAVILR GHLRMAGHSL GRCDIKDLPK EITVATSRTL SYYKLGASQR VASDSGFAVY
NRYRIGNGKL NTDHSSSSDN IALLVQ

Sequence alignment of the new M protein designs (COV_M_T2_3, COV_M_T2_4, COV_M_T2_5) with the previous M protein designs (COV_M_T1_1, COV_M_T2_1, COV_M_T2_2) is shown below:

| | | |
|---|---|---|
| COV_M_T1_1 | MADSNGTITVEELKKLLEQWNLVIGFLFLTWICLLQFAYANRNRFLYLIKLEFLWLLWPVTLA | 63 |
| COV_M_T2_3 | MADSNGTITVEELKKLLEQ-------------------------------------------- | 19 |
| COV_M_T2_1 | MAD-NGTITVEELKQLLEQWNLVIGFLFLAWIMLLQFAYANRNRFLYLVKLEFLWLLWPVTLA | 62 |
| COV_M_T2_4 | MAD-NGTITVEELKQLLEQ-------------------------------------------- | 18 |
| COV_M_T2_2 | MADSNGTITVEELKKLLEQWNLVIGFLFLTWICLLQFAYANRNRFLYLIKLEFLWLLWPVTLA | 63 |
| COV_M_T2_5 | MADSNGTITVEELKKLLEQ-------------------------------------------- | 19 |
| COV_M_T1_1 | CFVLAAVYRINWITGGIATAMACLVGLMWLSYFIASFRLFARTRSMWSFNPETNILLNVPLHG | 126 |
| COV_M_T2_3 | -------------TGGIATAMACLVGLMWLSYFIASFRLFARTRSMWSFNPETNILLNVPLHG | 70 |
| COV_M_T2_1 | CFVLAAVYRINWVTGGIATAMACLVGLMWLSYFVASFRLFARTRSMWSFNPETNILLNVPLRG | 125 |
| COV_M_T2_4 | CFVLAAVYRINWVTGGIATAMACLVGLMWLSYFVASFRLFARTRSMWSFNPETNILLNVPLRG | 69 |
| COV_M_T2_2 | CFVLAAVYRINWVTGGIATAMACLVGLMWLSYFVASFRLFARTRSMWSFNPETNILLNVPLRG | 126 |
| COV_M_T2_5 | ------------VTGGIATAMACLVGLMWLSYFVASFRLFARTRSMWSFNPETNILLNVPLRG | 70 |
| COV_M_T1_1 | TILTRPLLESELVIGAVILRGHLRIAGHHLGRCDIKDLPKEITVATSRTLSYYKLGASQRVAG | 189 |
| COV_M_T2_3 | TILTRPLLESELVIGAVILRGHLRIAGHHLGRCDIKDLPKEITVATSRTLSYYKLGASQRVAG | 133 |
| COV_M_T2_1 | TILTRPLMESELVIGAVIIRGHLAMAGHSLGRCDIKDLPKEITVATSRTLSYYKLGASQRVGT | 188 |
| COV_M_T2_4 | TILTRPLMESELVIGAVIIRGHLRMAGHSLGRCDIKDLPKEITVATSRTLSYYKLGASQRVGT | 132 |
| COV_M_T2_2 | SIITRPLMESELVIGAVILRGHLRMAGHSLGRCDIKDLPKEITVATSRTLSYYKLGASQRVAS | 189 |
| COV_M_T2_5 | SIITRPLMESELVIGAVILRGHLRMAGHSLGRCDIKDLPKEITVATSRTLSYYKLGASQRVAS | 133 |
| COV_M_T1_1 | DSGFAAYSRYRIGNYKENTQNSSSSDNIALLVQ | 222 |
| COV_M_T2_3 | DSGFAAYSRYRIGNGKENTDHSSSSDNIALLVQ | 166 |
| COV_M_T2_1 | DSGFAAYNRYRIGNYKENTDHAGSNDNIALLVQ | 221 |
| COV_M_T2_4 | DSGFAAYNRYRIGNGKENTDHAGSNDNIALLVQ | 165 |
| COV_M_T2_2 | DSGFAVYNRYRIGNYKENTQNSSSSDNIALLVQ | 222 |
| COV_M_T2_5 | DSGFAVYNRYRIGNGKLNTDH5555DNIALLVQ | 166 |

The amino acid differences of the designed sequences from the SARS2 M protein reference sequence are shown in the table below (with differences from the reference sequence highlighted in bold):

| | SARS2 Reference | COV_M_T2_1 | COV_M_T2_2 | COV_M_T2_3 | COV_M_T2_4 | COV_M_T2_5 |
|---|---|---|---|---|---|---|
| SARS2 M protein | Amino acid residue | Amino acid | Amino acid | Amino acid | Amino acid | Amino acid |
| residue position | (COV_M_T1_1) | residue | residue | residue | residue | residue |
| 4 | S | Deleted | S | S | Deleted | S |
| 15 | K | Q | K | K | Q | K |
| 20-75 | | | | Deleted | Deleted | Deleted |
| 30 | T | A | T | | | |
| 33 | C | M | C | | | |
| 40 | A | S | S | | | |
| 52 | I | V | I | | | |
| 76 | I | V | V | I | V | V |
| 87 | L | I | I | L | I | I |
| 97 | I | V | V | I | V | V |
| 125 | H | R | R | H | R | R |
| 127 | T | T | S | T | T | S |
| 129 | L | L | I | L | L | I |
| 134 | L | M | M | L | M | M |
| 145 | L | I | L | L | I | L |
| 151 | I | M | M | 1 | M | M |
| 155 | H | S | S | H | S | S |
| 188 | A | G | A | A | G | A |
| 189 | G | T | S | G | T | S |
| 195 | A | A | V | A | A | V |
| 197 | S | N | N | S | N | N |
| 204 | Y | Y | Y | G | G | G |
| 211 | S | A | S | S | A | S |
| 212 | S | G | S | S | G | S |
| 214 | S | N | S | S | N | S |

### EXAMPLE 28

### Glycosylation of S Protein RBD Proteins

FIG. 21 shows the spectra overlap (MALDI MS) of supernatants derived from HEK cells transfected with pEVAC plasmid encoding the following S protein RBD sequences:
COV_S_T2_5 (wild-type SARS1 RBD)
COV_S_T2_6 (wild-type SARS2 RBD)
COV_S_T2_13
COV_S_T2_14
COV_S_T2_15
COV_S_T2_16
COV_S_T2_17
COV_S_T2_18
COV_S_T2_19
COV_S_T2_20
M7 RBD
TM RBD

The results show that the RBD is peaking at 25-26 KDa, and a second peak appears at 29 KDa.

FIG. 22 shows the spectra for the following examples of recombinant RBD proteins:
RBD (one sample labelled “LMB”);
His-tagged RBD;
Another RBD protein sample labelled “Ralph”.

The amino acid sequence of COV_S_T2_19 is below:

>COV_S_T2_19
(SEQ ID NO: 55)
RVAPTKEVVRFPNITNLCPFGEVFNATRFPSVYAWERKRISNCVA
DYSVLYNSTSFSTFKCY GVSPTKLNDLCFTNVYADSFVIRGDEV
RQIAPGQTGVIADYNYKLPDDFTGCVIAWNTNNLDSTTGGNYNYL
YRSLRKSKLKPFERDISSDIYSPGGKPCSGVEGFNCYYPLRSYGF
FPTNGVGYQPYRVVVLSFELLNAPATVCGPKLSTDGGGGSGGGGS
GGGGSGGGGSKSSIASFFFIIGLIIGLFLVLRVGIHLCIKLKHTK
KRQIYTDIEMNRLGK

The amino acid sequence of COV_S_T2_20 is below:

>COV_S_T2_20
(SEQ ID NO: 56)
RVAPTKEVVRFPNITNLCPFGEVFNATKFPSVYAWERKKISNCVA
DYSVLYNSTSFSTFKCY GVSPTKLNDLCFTNVYADSFVIRGDEV
RQIAPGQTGVIADYNYKLPDDFTGCVIAWNTNNIDSTTGGNYNYL
YRSLRKSKLKPFERDISSDIYSPGGKPCSGVEGFNCYYPLRSYGF
FPTNGTGYQPYRVVVLSFELLNAPATVCGPKLSTDGGGGSGGGGS
GGGGSGGGGSKSSIASFFFIIGLIIGLFLVLRVGIHLCIKLKHTK
KRQIYTDIEMNRLGK

COV_S_T2_19 is essentially COV_S_T2_13 with a transmembrane domain, and COV_S_T2_20 is COV_S_T2_17 with a transmembrane domain.

The amino acid sequence of RBD protein (Leader-RBD-Tag) is below:

(SEQ ID NO: 51)
MKRGLCCVLLLCGAVFVSPSAARVQPTESIVRFPNITNLCPFGEV
FNATRFASVYAWNRKRISNCVADYSVLYNSASFSTFKCYGVSPTK
LNDLCFTNVYADSFVIRGDEVRQIAPGQTGKIADYNYKLPDDFTG
CVIAWNSNNLDSKVGGNYNYLYRLFRKSNLKPFERDISTEIYQAG
STPCNGVEGFNCYFPLQSYGFQPTNGVGYQPYRVVVLSFELLHAP
ATVCGPKKSTNGGSGLNDIFEAQKIEWHEGSHHHHHH

FIG. 22 shows that the LMB and His-tagged RBD proteins peak at ˜26 KDa (LMB is the higher peak in the figure), and that the Ralph RBD sample peaks at ˜31-32 KDa. Peaks are also seen at ˜52 KDa for “LMB” and “his RBD” (LMB is the higher peak), and at ˜62-64 KDa for the Ralph RBD sample.

It was concluded from these results that there are two main glycosylated forms of the proteins obtained from the supernatant, in comparison to purified (recombinant) protein. The purified protein is non-glycosylated or sparsely glycosylated. This difference in glycosylation is believed to be important, as the glycosylation sites surround the epitope region and are conserved in most sarbecoviruses. These glycosylation sites are also important for interaction with some of the antibodies.

FIG. 23 provides a reference for glycosylation of the “S” Spike protein. As can be seen from the spectra, the glycosylation pattern of the spike protein is mixed. On average, the mass for each glycan is ˜2 kDa. There are three sites of glycosylation for four of the S protein RBD designs (COV_T2_13, COV_T2_14, COV_T2_15, and COV_T2_16) and wild-type SARS1 RBD, two for wild-type SARS2 RBD, and four for S protein RBD designs COV_T2_17, COV_T2_18.

The mass of “Ralf RBD protein” is 29.2 kDa. The mass of the designed RBD proteins, and wild-type RBD is ˜24 kDa.

### EXAMPLE 29

### Pan-Sarbecovirus Vaccine Coverage

Pan-Sarbecovirus protection: Beta-Coronaviruses including SARS-CoV-2 (SARS2), -1 (SARS1) & the many Bat SARSr-COV (ACE2 receptor using) that threaten to spillover into humans.

FIG. 24 illustrates antigenic coverage achieved by universal Sarbecovirus B-cell and T-cell antigen targets. Part 1 shows Sarbecoviruses with the SARS1 and SARS2 clades highlighted along with human or bat host species. Part 2 shows machine learning predicted MHC class II binding (higher is stronger binding) of predicted epitopes within the insert. Lighter grey is for epitopes conserved within SARS2, darker grey are epitopes grafted in from other Sarbecoviruses such as SARS1.

### EXAMPLE 30

### Designed S Protein Sequence to Protect Against COVID-19 Variants

Multiple SARS-CoV-2 variants are circulating globally. Several new variants emerged in the fall of 2020, most notably:

In the United Kingdom (UK), a new variant of SARS-CoV-2 (known as 201/501Y.V1, VOC 202012/01, or B.1.1.7) emerged with a large number of mutations. This variant has since been detected in numerous countries around the world, including the United States (US). In January 2021, scientists from UK reported evidence that suggests the B.1.1.7 variant may be associated with an increased risk of death compared with other variants, although more studies are needed to confirm this finding. This variant was reported in the US at the end of December 2020.

In South Africa, another variant of SARS-CoV-2 (known as 20H/501Y.V2 or B. 1.351) emerged independently of B.1.1.7. This variant shares some mutations with B.1.1.7. Cases attributed to this variant have been detected in multiple countries outside of South Africa. This variant was reported in the US at the end of January 2021.

In Brazil, a variant of SARS-CoV-2 (known as P.1) emerged that was first was identified in four travelers from Brazil, who were tested during routine screening at Haneda airport outside Tokyo, Japan. This variant has 17 unique mutations, including three in the receptor binding domain of the spike protein. This variant was detected in the US at the end of January 2021.

Scientists are working to learn more about these variants to better understand how easily they might be transmitted and the effectiveness of currently authorized vaccines against them. New information about the virologic, epidemiologic, and clinical characteristics of these variants is rapidly emerging.

### B.1.1.7 Lineage (a.k.a. 201/501Y.V1 Variant of Concern (VOC) 202012/01)

This variant has a mutation in the receptor binding domain (RBD) of the spike protein at position 501, where the amino acid asparagine (N) has been replaced with tyrosine (Y). The shorthand for this mutation is N501Y. This variant also has several other mutations, including:
69/70 deletion: occurred spontaneously many times and likely leads to a conformational change in the spike protein.
P681H: near the S1/S2 furin cleavage site, a site with high variability in coronaviruses. This mutation has also emerged spontaneously multiple times.

This variant is estimated to have first emerged in the UK during September 2020.

Since Dec. 20, 2020, several countries have reported cases of the B.1.1.7 lineage, including the United States.

This variant is associated with increased transmissibility (i.e., more efficient and rapid transmission).

In January 2021, scientists from UK reported evidence (Horby P, Huntley C, Davies N, et al. NERVTAG note on B.1.1.7 severity. SAGE meeting report. Jan. 21, 2021) that suggests the B.1.1.7 variant may be associated with an increased risk of death compared with other variants.

Early reports found no evidence to suggest that the variant has any impact on the severity of disease or vaccine efficacy (Wu K, Werner A P, Moliva J I, et al. mRNA-1273 vaccine induces neutralizing antibodies against spike mutants from global SARS-CoV-2 variants. bioRxiv. Posted Jan. 25, 2021; Xie X, Zou J, Fontes-Garfias C R, et al. Neutralization of N501Y mutant SARS-CoV-2 by BNT162b2 vaccine-elicited sera. bioRxiv. Posted Jan. 7, 2021; Greaney A J, Loes A N, Crawford K H D, et al. Comprehensive mapping of mutations to the SARS-CoV-2 receptor-binding domain that affect recognition by polyclonal human serum antibodies. bioRxiv. [Preprint posted online Jan. 4, 2021]; Weisblum Y, Schmidt F, Zhang F, et al. Escape from neutralizing antibodies by SARS-CoV-2 spike protein variants. eLife 2020; 9:e61312.)

### B.1.351 Lineage (a.k.a. 20H/501Y.V2)

This variant has multiple mutations in the spike protein, including K417N, E484K, N501Y. Unlike the B.1.1.7 lineage detected in the UK, this variant does not contain the deletion at 69/70.

This variant was first identified in Nelson Mandela Bay, South Africa, in samples dating back to the beginning of October 2020, and cases have since been detected outside of South Africa, including the United States.

The variant also was identified in Zambia in late December 2020, at which time it appeared to be the predominant variant in the country.

Currently there is no evidence to suggest that this variant has any impact on disease severity.

There is some evidence to indicate that one of the spike protein mutations, E484K, may affect neutralization by some polyclonal and monoclonal antibodies (Weisblum Y, Schmidt F, Zhang F, et al. Escape from neutralizing antibodies by SARS-CoV-2 spike protein variants. eLife 2020; 9:e61312; Resende P C, Bezerra J F, de Vasconcelos R H T, at al. Spike E484K mutation in the first SARS-CoV-2 reinfection case confirmed in Brazil, 2020. [Posted on www.virological.org on Jan. 10, 2021])

### P.1 Lineage (a.k.a. 20J/501Y.V3)

The P.1 variant is a branch off the B.1.1.28 lineage that was first reported by the National Institute of Infectious Diseases (NIID) in Japan in four travelers from Brazil, sampled during routine screening at Haneda airport outside Tokyo.

The P.1 lineage contains three mutations in the spike protein receptor binding domain: K417T, E484K, and N501Y.

There is evidence to suggest that some of the mutations in the P.1 variant may affect its transmissibility and antigenic profile, which may affect the ability of antibodies generated through a previous natural infection or through vaccination to recognize and neutralize the virus.-A recent study reported on a cluster of cases in Manaus, the largest city in the Amazon region, in which the P.1 variant was identified in 42% of the specimens sequenced from late December (Resende P C, Bezerra J F, de Vasconcelos R H T, at al. Spike E484K mutation in the first SARS-CoV-2 reinfection case confirmed in Brazil, 2020. [Posted on www.virological.org on Jan. 10, 2021]). In this region, it is estimated that approximately 75% of the population had been infected with SARS-CoV2 as of October 2020. However, since mid-December the region has observed a surge in cases. The emergence of this variant raises concerns of a potential increase in transmissibility or propensity for SARS-CoV-2 re-infection of individuals.

This variant was identified in the United States at the end of January 2021.

One specific mutation, called D614G, is shared by these three variants. It gives the variants the ability to spread more quickly than the predominant viruses, as described in a non-peer-reviewed preprint article (1Bin Zhou, Tran Thi Nhu Thao, Donata Hoffmann, et al. SARS-CoV-2 spike D614G variant confers enhanced replication and transmissibility bioRxiv 2020.10.27 doi: https://doi.org/10.1101/2020.10.27.357558; Volz E, Hill V, McCrone J, et al. Evaluating the Effects of SARS-CoV-2 Spike Mutation D614G on Transmissibility and Pathogenicity. Cell 2021; 184 (64-75). doi: https://doi.org/10.1016/j.cell.2020.11.020). There also is epidemiologic evidence that variants with this specific mutation spread more quickly than viruses without the mutation (Korber B, Fischer W M, Gnanakaran S, et al. Tracking Changes in SARS-CoV-2 Spike: Evidence that D614G Increases Infectivity of the COVID-19 Virus. Cell 2021; 182 (812-7). doi: https://doi.org/10.1016/j.cell.2020.06.043). This mutation was one of the first documented in the US in the initial stages of the pandemic, after having initially circulated in Europe (Yurkovetskiy L, Wang X, Pascal K E, et al. Structural and Functional Analysis of the D614G SARS-CoV-2 Spike Protein Variant. Cell 2020; 183(3): 739-1. doi: https://doi.org/10.1016/j.cell.2020.09.032).

The variants are summarised in the table below (https://www.cdc.gov/coronavirus/2019-ncov/cases-updates/variant-surveillance/variant-info.html):

| | | | Countries | | Trans- |
|---|---|---|---|---|---|
| | Name | First | Reporting | Key | missibility |
| Name | (Nextstrain) | Detected | Cases | Mutations | Rate |
| B.1.1.7 | 20I/501Y.V1 | United | 70 | 69/70 | ~50% |
| | | Kingdom | | deletion | increase |
| | | | | 144Y | |
| | | | | deletion | |
| | | | | N501Y | |
| | | | | A570D | |
| | | | | D614G | |
| | | | | P681H | |
| P.1 | 20J/501Y.V3 | Japan/ | >4 | E484K | Not |
| | | Brazil | | K417N/T | determined |
| | | | | N501Y | |
| | | | | D614G | |
| B.1.351 | 20H/501.V2 | South | >30 | K417N | Not |
| | | Africa | | E484K | determined |
| | | | | N501Y | |
| | | | | D614G | |

We have designed a new full-length S protein sequence (referred to as “VOC Chimera”, or COV_S_T2_29) for use as a COVID-19 vaccine insert to protect against variants B.1.1.7, P.1, and B.1.351.

The full-length S protein amino acid sequence of SARS_COV_2 isolate EPI_ISL_402130 (a reference sequence) is given below:

>EPI_ISL_402130 (Wuhan strain)
(SEQ ID NO: 52)
MFVFLVLLPL VSSQCVNLTT RTQLPPAYTN SFTRGVYYPD KVFRSSVLHS TQDLFLPFFS 60
NVTWFHAIHV SGTNGTKRED NPVLPENDGV YFASTEKSNI IRGWIFGTTL DSKTQSLLIV 120
NNATNVVIKV CEFQFCNDPF LGVYYHKNNK SWMESEFRVY SSANNCTFEY VSQPELMDLE 180
GKQGNFKNLR EFVFKNIDGY FKIYSKHTPI NLVRDLPQGF SALEPLVDLP IGINITRFQT 240
LLALHRSYLT PGDSSSGWTA GAAAYYVGYL QPRTELLKYN ENGTITDAVD CALDPLSETK 300
CTLKSFTVEK GIYQTSNERV QPTESIVRFP NITNLCPFGE VENATRFASV YAWNRKRISN 360
CVADYSVLYN SASFSTEKCY GVSPTKLNDL CFTNVYADSF VIRGDEVRQI APGQTGKIAD 420
YNYKLPDDFT GCVIAWNSNN LDSKVGGNYN YLYRLERKSN LKPFERDIST EIYQAGSTPC 480
NGVEGENCYF PLQSYGFQPT NGVGYQPYRV VVLSFELLHA PATVCGPKKS TNLVKNKCVN 540
FNFNGLTGTG VLTESNKKEL PFQQFGRDIA DTTDAVRDPQ TLEILDITPC SFGGVSVITP 600
GTNTSNQVAV LYQDVNCTEV PVAIHADQLT PTWRVYSTGS NVFQTRAGCL IGAEHVNNSY 660
ECDIPIGAGI CASYQTQTNS PRRARSVASQ SIIAYTMSLG AENSVAYSNN SIAIPTNFTI 720
SVTTEILPVS MTKTSVDCTM YICGDSTECS NLLLQYGSFC TQLNRALTGI AVEQDKNTQE 780
VFAQVKQIYK TPPIKDEGGF NFSQILPDPS KPSKRSFIED LLENKVTLAD AGFIKQYGDC 840
LGDIAARDLI CAQKENGLTV LPPLLTDEMI AQYTSALLAG TITSGWTFGA GAALQIPFAM 900
QMAYRENGIG VTQNVLYENQ KLIANQFNSA IGKIQDSLSS TASALGKLQD VVNQNAQALN 960
TLVKQLSSNF GAISSVLNDI LSRLDKVEAE VQIDRLITGR LQSLQTYVTQ QLIRAAEIRA 1020
SANLAATKMS ECVLGQSKRV DECGKGYHLM SFPQSAPHGV VFLHVTYVPA QEKNFTTAPA 1080
ICHDGKAHEP REGVFVSNGT HWFVTQRNFY EPQIITTDNT FVSGNCDVVI GIVNNTVYDP 1140
LQPELDSFKE ELDKYFKNHT SPDVDLGDIS GINASVVNIQ KEIDRLNEVA KNLNESLIDL 1200
QELGKYEQYI KWPWYIWLGF IAGLIAIVMV TIMLCCMTSC CSCLKGCCSC GSCCKEDEDD 1260
SEPVLKGVKL HYT 1273

The amino acid sequence of the designed full-length S protein sequence is given below:

COV_S_T2_29 (VOC chimera)
(SEQ ID NO: 53)
MFVFLVLLPL VSSQCVNFTN RTQLPSAYIN SFTRGVYYPD KVERSSVLHS TQDLFLPFFS 60
NVTWFHAISG TNGTKRFDNP VLPENDGVYF ASTEKSNIIR GWIFGTTLDS KTQSLLIVNN 120
ATNVVIKVCE FQFCNDPFLG VYHKNNKSWM ESEFRVYSSA NNCTFEYVSQ PFLMDLEGKQ 180
GNFKNLREFV FKNIDGYFKI YSKHTPINLV RDLPQGESAL EPLVDLPIGI NITRFQTLLA 240
LHRSYLTPGD SSSGWTAGAA AYYVGYLQPR TELLKYNENG TITDAVDCAL DPLSETKCTL 300
KSFTVEKGIY QTSNERVQPT ESIVREPNIT NLCPFGEVEN ATRFASVYAW NRKRISNCVA 360
DYSVLYNSAS FSTFKCYGVS PTKLNDLCFT NVYADSFVIR GDEVRQIAPG QTGNIADYNY 420
KLPDDETGCV IAWNSNNLDS KVGGNYNYLY RLERKSNLKP FERDISTEIY QAGSTPCNGV 480
KGENCYFPLQ SYGFQPTYGV GYQPYRVVVL SFELLHAPAT VCGPKKSTNL VKNKCVNENE 540
NGLTGTGVLT ESNKKELPFQ QFGRDIADTT DAVRDPQTLE ILDITPCSFG GVSVITPGTN 600
TSNQVAVLYQ GVNCTEVPVA IHADQLTPTW RVYSTGSNVF QTRAGCLIGA EHVNNSYECD 660
IPIGAGICAS YQTQTNSHRR ARSVASQSII AYTMSLGAEN SVAYSNNSIA IPTNFTISVT 720
TEILPVSMTK TSVDCTMYIC GDSTECSNLL LQYGSFCTQL NRALTGIAVE QDKNTQEVFA 780
QVKQIYKTPP IKDEGGENFS QILPDPSKPS KRSFIEDLLF NKVTLADAGF IKQYGDCLGD 840
IAARDLICAQ KENGLTVLPP LLTDEMIAQY TSALLAGTIT SGWTFGAGAA LQIPFAMQMA 900
YRENGIGVTQ NVLYENQKLI ANQFNSAIGK IQDSLSSTAS ALGKLQDVVN QNAQALNTLV 960
KQLSSNFGAI SSVLNDILSR LDPPEAEVQI DRLITGRLQS LQTYVTQQLI RAAEIRASAN 1020
LAATKMSECV LGQSKRVDFC GKGYHLMSFP QSAPHGVVFL HVTYVPAQEK NFTTAPAICH 1080
DGKAHFPREG VFVSNGTHWF VTQRNFYEPQ IITTDNTFVS GNCDVVIGIV NNTVYDPLQP 1140
ELDSFKEELD KYFKNHTSPD VDLGDISGIN ASVVNIQKEI DRLNEVAKNL NESLIDLQEL 1200
GKYEQYIKWP WYIWLGFIAG LIAIVMVTIM LCCMTSCCSC LKGCCSCGSC CKEDEDDSEP 1260
VLKGVKLHYT 1270

Alignment of these two sequences is shown below. The amino acid differences between the sequences are shown boxed, with the two amino acid changes made to provide structure stability shown in the shaded box.

The amino acid differences of the designed sequence COV_S_T2_29 from the SARS2 S protein reference sequence (EPI_ISL_402130_Wuhan strain) are summarised in the table below.

| SARS2 S protein | SARS2 Reference | COV_S_T2_29 |
|---|---|---|
| residue position | amino acid residue | amino acid residue |
| (SEQ ID NO: 52) | (SEQ ID NO: 52) | (SEQ ID NO: 53) |
| 18 | L | F |
| 20 | T | N |
| 26 | P | S |
| 69 | H | Deletion |
| 70 | V | Deletion |
| 144 | Y | Deletion |
| 417 | K | N |
| 484 | E | K |
| 501 | N | Y |
| 614 | D | G |
| 681 | P | H |
| 986 | K | P |
| 987 | V | P |

| | | |
|---|---|---|
| EPI_ISL_ 402130_ [symbol] COV_S_T2_29 | 1 1 | 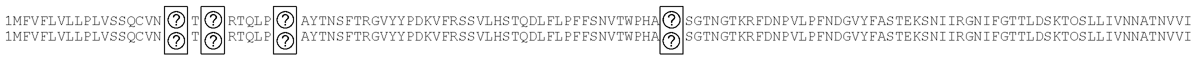 |
| EPI_ISL_ 402130_ [symbol] . COV_S_T2_29 | 129 127 | 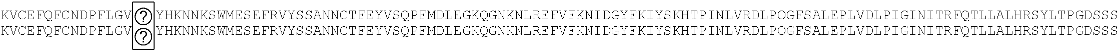 |
| EPI_ISL_ | 257 | GVVTAGAAAYVGLQPRTFLLKYNENGTITDAVDCALDPLSETKCTLKSFTVEKGIYDTSNFRVQPTESIVRFPNITNLCPFGEVFNATRFASVYANNRKRISNCVADYSVLYNSASTFSTFKCYGVSP |
| 402130_ | 254 | GVVTAGAAAYYVGYLQFRTFLLKYNENGTITDAVDCALDPLSETKCTLKSFTVEKGIYDTSNFRVQPTESIVRFPNITNLCPFGEVFNATRFASVYANNRKRISNGYADYSVLVNEASFSTFKCYDVSP |
| Wuha. | | |
| COV_S_T2_29 | | |
| EPI_ISL_ 407130_ Wuha. COV_S_T2_29 | 385 382 | 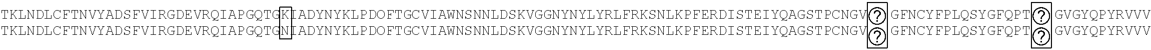 |
| EPI_ISL_ 402130_ Wuha. COV_S_T2_29 | 513 510 | 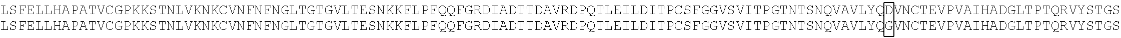 |
| EPI_ISL_ 402130_ Wuha. COV_S_T2_29 | 641 638 | 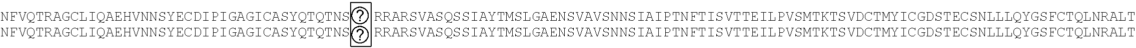 |
| EPI_ISL_ | 769 | DIAVEQDKNTQEVFADVKQIYKTPPIKDFGGFNHFSQILPDFSKPSKRSFIEDLLFNKVTLADAGFIKQYGDCLGDIAARDLICAQKFNDLTVLPPLLTDEMIAQYTSALLAGTITSGVVTFGAGAALQI |
| 401230_ | 766 | GIAVEQDKNTQEVFAGVKQIYKTPPIKDFGGFNFSQILPDPSKPSKRSFIEDLLFNKVTLADAGFIKQYGDCLGDIAARDLICAGKFNGLTVLPPLLTDEMIAQYTGALLAGTITSGATFGAGAALDI |
| Wuha. | | |
| COV_S_T2_29 | | |
| EPI_ISL_ 402130_ Wuha. COV_S_T2_29 | 897 894 | 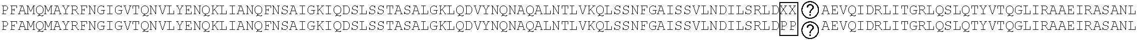 |
| EPI_ISL_ | 1025 | AATKMSECVLGDSKRVDFCGKGYHLMSFPQSAPHGVVFLHVTVVPAQEKNIFTTAPAICHDGKAHFPREGVFVSNGTHAFVTQRNFVEPQIITTDNTFVSGNCDVVIGIYNNTVYDPLQPELDSFKEEL |
| 402130_ | 1022 | AATKMSECVLGQSKRYDFCGKGYHLMSFPQSAPHGVVFLHVTYYPAEKNFTTAPAICHDGKAHFREQYFVSNGTHAFVTQRNFYEPQIITTDNTFYSGNCDVVIGIVNNTVYDPLGPELDSFKEEL |
| Wuha. | | |
| COV_S_T2_29 | | |
| EPI_ISL_ | 1153 | DKYFKNHTSPDYDLGDISGINASVVNIQKEIDRLNEVAKNLNESLIDLQELGKYEQYIKAPWYIWLGFIAGLIAIVMVTIMLQCMTSCCSCLKGCCSCGSCLKFDEDDSEPVLKGVKLHYT |
| 402130_ | 1150 | DKYFKNHTSPDVGLGDISGINASVVNIQKEIDRLNEVAKNLNESLIDLQELGKYEGYIKAPVVYIWLGFIAGLIAIVMVTIMLCCMTSCCSCLKGCCSQSCDKFDEDDSEPVLKGSVKLGVKLMYT |
| Wuha. | | |
| COV_S_T2_29 | | |

[symbol]

indicates data missing or illegible when filed

### EXAMPLE 31

### Designed S Protein Sequence in Closed State to Protect Against Known COVID-19 Variants, And Predicted Future Variants

The majority of SARS-CoV-2 vaccines in use or in advanced clinical development are based on the viral spike protein(S) as their immunogen. S is present on virions as pre-fusion trimers in which the receptor binding domain (RBD) is stochastically open or closed. Neutralizing antibodies have been described that act against both open and closed conformations. The long-term success of vaccination strategies will depend upon inducing antibodies that provide long-lasting broad immunity against evolving, circulating SARS-CoV-2 strains, while avoiding the risk of antibody dependent enhancement as observed with other Coronavirus vaccines.

Carnell et al. (“SARS-CoV-2 spike protein arrested in the closed state induces potent neutralizing responses”; https://doi.org/10.1101/2021.01.14.426695, posted 14 Jan. 2021) have assessed the results of immunization in a mouse model using an S protein trimer that is arrested in the closed state to prevent exposure of the receptor binding site and therefore interaction with the receptor. The authors compared this with a range of other modified S protein constructs, including representatives used in current vaccines. They found that all trimeric S proteins induce a long-lived, strongly neutralizing antibody response as well as T-cell responses. Notably, the protein binding properties of sera induced by the closed spike differed from those induced by standard S protein constructs. Closed S proteins induced more potent neutralising responses than expected based on the degree to which they inhibit interactions between the RBD and ACE2. The authors conclude that these observations suggest that closed spikes recruit different, but equally potent, virus-inhibiting immune responses than open spikes, and that this is likely to include neutralizing antibodies against conformational epitopes present in the closed conformation.

We have appreciated that the amino acid changes of the designed S protein sequences disclosed herein (and especially in Example 30 above) may optionally be present in a designed S protein that is arrested in the closed state, and thereby further improve the antibody response of the designed sequences. In particular, use of such structural constraints may reduce immunodominance to key regions, and spread the antibody response to focus on other, or less immunodominant sites.

SARS-CoV-2 is continually evolving, with more contagious mutations spreading rapidly. Zahradník et al., 2021 (“SARS-CoV-2 RBD in vitro evolution follows contagious mutation spread, yet generates an able infection inhibitor”; doi: https://doi.org/10.1101/2021.01.06.425392, posted 29 Jan. 2021) recently reported using in vitro evolution to affinity maturate the receptor-binding domain (RBD) of the spike protein towards ACE2 resulting in the more contagious mutations, S477N, E484K, and N501Y, to be among the first selected, explaining the convergent evolution of the “European” (20E-EU1), “British” (501.V1), “South African” (501.V2), and “Brazilian” variants (501.V3). The authors report that further in vitro evolution enhancing binding by 600-fold provides guidelines towards potentially new evolving mutations with even higher infectivity. For example, Q498R epistatic to N501Y.

We have also appreciated that the designed S protein sequences (RBD, truncated, or full-length) disclosed herein (and especially in Example 30 above) may optionally also include amino acid substitutions at residue positions predicted to be mutated in future COVID-19 variants with a vaccine escape response.

The amino acid sequence alignment below shows the full-length S protein amino acid sequence of SARS_COV_2 isolate EPI_ISL_402130 (a reference sequence; SEQ ID NO:52) with the amino acid changes made for the designed S protein sequence described in Example 30 above (“VOC Chimera”, or COV_S_T2_29; SEQ ID NO:53), shown underneath the isolate sequence (in the line referred to as “Super_spike”). This designed (“Super_spike”) S protein sequence may optionally also include one or more amino acid changes (a substitution or deletion) at one or more of the residue positions predicted to be mutated in future COVID-19 variants with a vaccine escape response.

The line underneath the “Super_spike” sequence alignment shows the residues that may be substituted for cysteine residues to allow formation of a disulphide bridge to form a “closed S protein”. These cysteine substitutions may be combined with one or more (or all) of the amino acid changes made in the designed S protein sequence of the “Super_spike” sequence (COV_S_T2_29; SEQ ID NO:53), and optionally with one or more (or all) amino acid changes at the residue positions predicted to be mutated in future COVID-19 variants with a vaccine escape response (especially including, for example, Q498R).

The table below the alignment summarises the amino acid changes.

The shaded residues in the alignment (and table) are as follows:
Grey—amino acid residues that have been changed in the “Super_spike” design;
Dark grey—amino acid residues that may be substituted for a cysteine residue to allow formation of a “closed S protein”;
Light grey—amino acid residues that have been predicted to be mutated in future COVID-19 variants and potentially generate a vaccine escape response.

| | |
|---|---|
| EPI_ISL_ 402130 Super_ spike X2 | 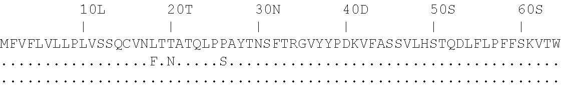 |
| EPI_ISL_ 402130 Super_ spike X2 | 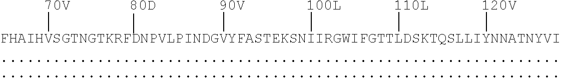 |
| EPI_ISL_ 402130 Super_ spike X2 | 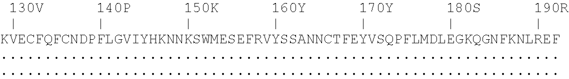 |
| EPI_ISL_ | 200Y 210I 220F 230P 240T 250T |
| 402130 | \| \| \| \| \| \| |
| Super_ | VFKNIDGYFKIYSKHTFINLVRDLPQGFSALEPLVDLPIGINITRFQTLLALHRSYLTPGDSSS |
| spike | ................................................................ |
| X2 | ................................................................ |
| EPI_ISL_ | 260A 270L 280K 290D 300K 310K |
| 402130 | \| \| \| \| \| \| |
| Super_ | GWTAGAAAYTVGYLQPHTFLLKYNENGTITDAVDCALDPLSETKCTLKSFTVEKGIYQTSKFRY |
| spike | ................................................................ |
| X2 | ................................................................ |
| EPI_ISL_ | 330P 340F 350V 360N 370N 380Y |
| 402130 | \| \| \| \| \| \| |
| Super_ | QPTESIVRFPKITNLCFFGEVFNATRFASVYAWNRKRISNCVADYSVLYNSASFSTFKCYCVSP |
| spike | ................................................................ |
| X2 | ................................................................ |
| EPI_ISL_ 402130 Super_ spike X2 | 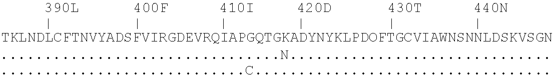 |
| EPI_ISL_ 402130 Super_ spike X2 | 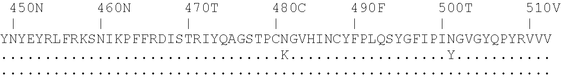 |
| EPI_ISL_ | 520A 530S 540N 550G 560K 570A |
| 402130 | \| \| \| \| \| \| |
| Super_ | LSFELLHAPATVCGPKKSTNLVKNKCVNFNFNGLTGTCVLTESNKKFLPFQQFQRDIADTTDAY |
| spike | ................................................................ |
| X2 | ................................................................ |
| EPI_ISL_ 402130 Super_ spike X2 | 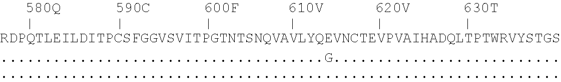 |
| EPI_ISL_ 402130 Super_ spike X2 | 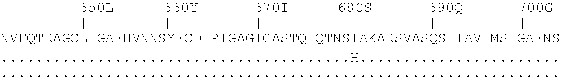 |
| EPI_ISL_ | 710N 720I 730S 740M 750S 760C |
| 402130 | \| \| \| \| \| \| |
| Super_ | VAYSNNSIAIPTNFTISVTTEILPVSMTKTSYDCTMYICDDSTECSNLLLQYGSFCTQLNRALT |
| spike | ................................................................ |
| X2 | ................................................................ |
| EPI_ISL_ | 770I 780E 790K 800F 810S 820D 830D |
| 402130 | \| \| \| \| \| \| \| |
| Super_ | GIAVEQDKNTQEVFAQVKQIYKTPPIKDFGGFNFSQILPDPSKPSKRSFIEDLLFNKVTLADAG |
| spike | ................................................................ |
| X2 | ................................................................ |
| EPI_ISL_ | 840C 850I 860V 870I 880G 890A |
| 402130 | \| \| \| \| \| \| |
| Super_ | FIKQYGDCLGDIAARDLICAQKFNGLTVLPPLLTDEMIAQYTSALLAGTITSGWTFGAGAALQI |
| spike | ................................................................ |
| X2 | ................................................................ |
| EPI_ISL_ | 900M 910G 920Q 930A 940S 950D |
| 402130 | \| \| \| \| \| \| |
| Super_ | PFAMGMAYEFNGIGVTQNVLYENQKLIANQFNSAIGKIQDSLSSYASALGKLQDVVNQNAQALN |
| spike | ................................................................ |
| X2 | ................................................................ |
| EPI_ISL_ 402130 Super_ spike X2 | 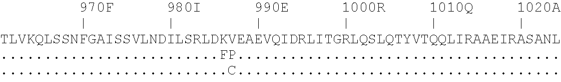 |
| EPI_ISL_ | 1030S 1040V 1050M 1060V 1070A 1080A |
| 402130 | \| \| \| \| \| \| |
| Super_ | AATKNSECVLGQSKRVDFCGKSYHLMSFPQSAPEGVVTLHVTYVPAQEKNFTTAPAICHDGKAH |
| spike | ................................................................ |
| X2 | ................................................................ |
| EPI_ISL_ | 1090P 1100T 1110Y 1120T 1130I 1140P 1150E |
| 402130 | \| \| \| \| \| \| \| |
| Super_ | FPREGVFVSNGTHWFVTQRNFYEPQIITYDNTFVSGNCDVVIGIVNNTVYDPLQPELDSFKEHL |
| spike | ................................................................ |
| X2 | ................................................................ |
| EPI_ISL_ | 1160T 1170S 1180Q 1190A 1200L 1210I |
| 402130 | \| \| \| \| \| \| |
| Super_ | DKYFKNHTSPDVDLGDISGINASVVNIQKEIDRLNEVAKNLNESLIDLQELGKYEQYIKWPWYI |
| spike | ................................................................ |
| X2 | ................................................................ |
| EPI_ISL_ | 1220F 1230V 1240C 1250C 1260D 1270L |
| 402130 | \| \| \| \| \| \| |
| Super_ | WLGTIAGLIAIVMVTIMLCCMTSCCSCLKGCCSCGSCCKFDEDDSEPVLKGVKLHYR |
| spike | ......................................................... |
| X2 | ......................................................... |

| | | | Residues at | |
|---|---|---|---|---|
| | | | which | Residue at |
| | | | mutations | which |
| | SARS2 | | have been | cysteine |
| SARS2 S | Reference | | predicted to | substitution |
| protein | amino | COV_S_T2_29 | arise and | can be made |
| residue | acid | amino acid | generate | to form |
| position | residue | residue | vaccine | closed |
| (SEQ ID | (SEQ ID | (SEQ ID | escape | spike |
| NO: 52) | NO: 52) | NO: 53) | response | protein |
| 18 | L | F | | |
| 20 | T | N | | |
| 26 | P | S | | |
| 69 | H | Deletion | | |
| 70 | V | Deletion | | |
| 144 | Y | Deletion | | |
| 413 | G | | | C |
| 417 | K | N | | |
| 446 | G | | * | |
| 452 | L | | * | |
| 477 | S | | * | |
| 484 | E | K | | |
| 498 | Q | | * (for | |
| | | | example | |
| | | | Q498R) | |
| 501 | N | Y | | |
| 614 | D | G | | |
| 681 | P | H | | |
| 986 | K | P | | |
| 987 | V | P | | C |

Optionally G413C and V987C is combined with one or more (or all) of the amino acid changes listed in the table below:

| SARS2 S protein | COV_S_T2_29 |
|---|---|
| residue position | amino acid residue |
| (SEQ ID NO: 52) | (SEQ ID NO: 53) |
| 18 | F |
| 20 | N |
| 26 | S |
| 69 | — (deletion) |
| 70 | — (deletion) |
| 144 | — (deletion) |
| 417 | N |
| 484 | K |
| 501 | Y |
| 614 | G |
| 681 | H |

A further amino acid change that may optionally be included is K986P.

### EXAMPLE 32

### Epitope Optimised Broad Coverage Vaccine Designs for Sarbecoviruses

### Overview

To increase the coverage of our receptor binding domain (RBD) based vaccine designs to all the extant sarbecovirus sub-genus of Beta-coronaviruses, a phylogenetically optimised vaccine design is constructed. This design is further used as backbone for designing both epitope optimised and immune re-focussed designs. The epitope information is derived largely from the known high-resolution structural data of spike protein-antibody complex. Few of these epitopes are reported to cross protect SARS-1 and SARS-2 and were included in the designs to increase the coverage of the vaccine designs. On further analysis of the sequence divergence of the epitopes, it was observed that one of the epitopes shows maximum divergence among sarbecovirus in comparison to other regions/epitopes of RBD. To enhance the immune response toward better conserved epitopes, post-translation modification-glycosylation was introduced at this epitope.

### Results

Design of Broad Coverage Vaccine Antigens

To achieve broader response towards sarbecoviruses, we first generated a phylogenetically optimised design (COV_S_T2_13) (SEQ ID NO:27) where the amino acid sequence of RBD is optimised for all the extant sequences represented in FIG. 35A. Such a design is expected to generate broader antibody response compared to individual antigen from the extant species. To further understand the contribution of each epitope to antibody response, we modified the epitope sequences of COV_S_T2_13 to match the epitope sequences from SARS-1 and SARS-2. Three conformational epitopes (also referred to herein as “discontinuous epitopes”) are identified through structural analyses of RBD-antibody complex (FIG. 35B). Two of these epitopes (henceforth termed as A and B) are reported to bind antibodies that neutralise both SARS-1 and SARS-2. These epitopes on COV_S_T2_13 designs are modified to match the SARS-1 epitope sequence (COV_S_T2_14 (SEQ ID NO: 28) and COV_S_T2_15 (SEQ ID NO:29)) to understand the contribution of these epitopes to generate neutralising antibody response against both SARS-1 and SARS-2. The third epitope (henceforth termed as C) is in and around the receptor binding region. This epitope shows maximum divergence (FIG. 35C) and is expected to generate a virus specific antibody response. To understand the importance of the amino acid composition of this epitope in generating neutralising antibody response, this epitope is modified to match the epitope from SARS-2 (COV_S_T2_16) (SEQ ID NO:30). Further to broaden the antibody response to both SARS-1 and SARS-2, a glycosylation site is introduced at the third epitope for both COV_S_T2_14 and COV_S_T2_15 (COV_S_T2_17 (SEQ ID NO:31) and COV_S_T2_18 (SEQ ID NO:32) respectively). To compare the efficacy in generating neutralising antibody response in soluble or membrane bound form, a membrane bound form for COV_S_T2_13 and COV_S_T2_17 (COV_S_T2_19 (SEQ ID NO:55) and COV_S_T2_20 (SEQ ID NO:56) respectively) is designed. All the designs are tabulated in the Table below. The sequence alignment of all the vaccine designs is shown in FIG. 37A. The residues that differ between the vaccine designs are boxed in black.

TABLE
Description of the vaccine designs used in the study.

| DESIGN | DESCRIPTION |
|---|---|
| COV_S_T2_13 | Phylogenetic optimised |
| COV_S_T2_14 | COV_S_T2_13 with epitope A and epitope B |
| COV_S_T2_15 | from SARS-1 |
| COV_S_T2_16 | COV_S_T2_13 with epitope C from SARS-2 |
| COV_S_T2_17 | COV_S_T2_13 with glycosylation site at |
| COV_S_T2_18 | epitope C |
| COV_S_T2_19 | Membrane bound version of COV_S_T2_13 |
| COV_S_T2_20 | Membrane bound version of COV_S_T2_17 |

FIG. 36(A) shows a Western Blot of sera from mice immunised with the vaccine designs. FIG. 36 (B) shows antibody binding responses of Cell Surface expression bleed 2.

### Neutralisation Data

Sera from mice injected with the vaccine designs (COV_S_T2-13-20), SARS-1 RBD and SARS-2 RBD are checked for neutralisation of SARS-1 and SARS-2 pseudotypes. As a positive control, human sera from an infected individual are used. The neutralisation curves are shown in FIG. 37B. The phylogenetically optimised design (COV_S_T2_13) could generate neutralising antibody against SARS-2 but not for SARS-1. On comparing the sequence of the COV_S_T2_13 with SARS-1 and SARS-2, it is observed that the epitope C was enriched with amino acids from SARS-2 in comparison to other sarbecoviruses represented in phylogenetic tree (FIG. 35A). Sera from mice vaccinated with COV_S_T2_14, COV_S_T2_15, and COV_S_T2_16 showed data like COV_S_T2_13 for SARS-1, suggesting strongly that the epitope C is an immunodominant epitope and epitope A and B are immune sub-dominant epitope. Better neutralisation of SARS-2 by COV_S_T2_16 in comparison to COV_S_T2_13 suggests that the mutations at epitope C can lead to lower neutralisation of SARS-2. Substitution made in COV_S_T2_15 enhances the immunogenic response for SARS-2. The difference in immunogenic response could be due to the substitution of a small amino acid serine by bulky phenylalanine group.

Sera from COV_T2_S_17 and COV_T2_S_18 designs could neutralise both SARS-1 and SARS-2, suggesting that the introduction of glycosylation at epitope C successfully focused the immune response towards epitope A and epitope B. Thus, validating our design strategy. Comparison of neutralisation data of COV_T2_S_13 and COV_T2_S_17 with COV_S_T2_19 and COV_S_T2_20 respectively suggest that the membrane bound and soluble form similar immunogenic response in mice.

Neutralisation data for bat viruses (not shown) shows broader coverage. This rationalises the usage of phylogenetic optimised sequence as the template for further designs.

Competition data (not shown) shows that all the designs generate antibodies that block receptor binding.

### Discussion

A vaccine design which can generate antibody response against diverse sarbecovirus is desirable. To achieve this, we first generated a novel protein sequence (COV_S_T2_13) for the receptor binding domain of the spike protein by using sequence information for all the know extant sarbecoviruses. Each amino acid position in the sequence is chosen based on the phylogenetic relatedness of the input sequences. The novel sequence generated neutralising response against SARS-2 but not much against SARS-1. On comparison of the epitopes in the COV_S_T2_13 and SARS-1 and SARS-2, it was observed that the epitopes were more biased towards SARS-2 compared to SARS-1. To expand the reactivity towards SARS-1, two of the epitopes (which were also conserved between SARS-1 and SARS-2) were mutated to match the sequence from SARS-1 (COV_S_T2_14 and COV_S_T2_15) and the third epitope was mutated to match SARS-2 (COV_S_T2_16). Comparison of the neutralisation from these designs suggested that the two conserved epitopes are sub-dominant in nature compared to the third epitope. Also, comparison of COV_S_T2_16 with COV_S_T2_13 suggested that conservative mutations in the third epitope can cause immune escape. To focus the immune response towards the conserved epitopes, a glycosylation site was introduced at the more diverged third epitope (COV_S_T2_17 and COV_S_T2_18). The introduction of the glycosylation site indeed broadened the immune response to both SARS-1 and SARS-2, with cross-neutralisation observed for both the designs. The data presented here strongly supports the design strategy to broaden the coverage of vaccine designs by re-focussing the immune response to better conserved epitopes by introducing modifications in epitopes that more diverged.

### Methodology

### Phylogenetic Analysis

Protein sequences of spike proteins were downloaded from the NCBI virus database for all the known sarbecoviruses. Multiple sequence alignment (MSA) was generated using the MUSCLE algorithm. The resulting MSA was pruned to the RBD region and used as input for phylogenetic tree reconstruction. The phylogenetic tree was generated using IQTREE algorithm using protein model with best AIC score. The resultant tree was used for generation of phylogenetically optimised design using FASTML algorithm.

### Epitope Identification

Available structural data for Spike protein-antibody complexes for SARS-1 and SARS-2 were downloaded from the Protein Databank (PDB). These structural data were further pruned for antigen-antibody complexes where the epitope region is in the RBD. Amino acid residues of antigen that have at least one atom within 5 Å radii of at least one atom of amino acid of antibody are defined as epitope residues. An epitope region is defined as contiguous stretch of at least 5 amino acids.

### Molecular Modelling

Structural models were generated for COV_S_T2_13 using MODELLER algorithm. The structural model with the highest DOPE score was chosen as the working model for the further molecular modelling. The side chains for the model were further optimised using SCWRL library and energy minimised using GROMACS package. Structural stability of the COV_S_T2_14-COV_S_T2_18 designs was checked for using POSSCAN and BUILD module of FOLDX algorithm using the optimised structural model of COV_S_T2_13.

### EXAMPLE 33

### Dose Finding Study of COV_S_T2_17 (SEQ ID NO:31), a Pan-Sarbeco Coronavirus Vaccine DNA Candidate, Delivered by Needleless Intradermal Administration

Study protocol in brief (FIG. 38):

To determine the optimal dose of DNA, a pre-clinical vaccine study was undertaken in mature Hartley Guinea pigs. Animals were randomised into six groups of eight animals and pre-bled to determine the absence of anti-SARS-CoV-2 antibodies.

Group 1 (control) group received the high dose of 400 μg (2 mg/ml) of the modified SARS-CoV-2 RBD COV_S_T2_8 DNA subcutaneously, to compare to a second group the same control DNA of COV_S_T2_8 at 400 μg administered intradermally (ID) by the PharmaJet Tropis device. The remaining four groups received the pan-Sarbeco vaccine candidate, COV_S_T2_17 at 100 μg (0.5 mg/ml), 200 μg (1 mg/ml) (two groups, one receiving 2, the other 3 doses) or 400 μg (400 μg/ml) intradermally at day 0 and 28. Animals were bled at days 14, 28, 42, 56 and 70.

### Elisa to Determine the Level of Antibodies to the RBD of SARS-CoV-2, and SARS (FIG. 39):

Panel A (left) Plates coated with SARS-CoV-2 RBD.

28 days following the first immunisation an ELISA assay was performed to determine the titre of anti-SARS-CoV-2 RBD, or anti-SARS RBD antibodies induced 28 days after one DNA immunisation. The top left panel (T2_8 at 400 μg sc) demonstrates the antibody responses to SARS-CoV-2 in 5 out of 8 animals, compared to the bottom right hand panel (T2_8 at 400 μg DNA administered ID by the Tropis Pharmajet) where 7 of 8 animals respond strongly to SARS-CoV-2 RBD. The 4 remaining groups receiving COV_S_T2_17 ID by PharmaJet delivery, showed similar anti-SARS-CoV-2 responses to 400 μg of the SARS-CoV-2 RBD DNA administered at the maximal dose.

Panel B (right) Plates coated with SARS RBD.

The same 28 day serum samples at serial dilutions were tested for binding to the SARS RBD.

The top left panel (T2_8 at 400 μg sc) demonstrates low titre antibodies, with only 2 of 8 animals reaching an OD of 0.5. The same dose of the SARS-CoV-2 RBD vaccine given by the PharmaJet device (bottom right hand panel) demonstrates slightly improved but weak cross-reactive responses to the SARS RBD in contrast to its homotypic response to the SARS-CoV-2 RBD (panel A, left). In contrast all of the pan-Sarbeco T2_17 groups respond strongly to the SARS RBD in a dose-dependent manor, with all animals in the high (400 μg) (bottom row left in panel B) and medium doses (200 μg) groups (middle row panel B) responding strongly, and a more variable but distinct response in all 8 animals in the lowest (100 μg) T2_17 group (top right, panel B).

Virus Neutralisation at Day 28 after 1 Immunisation (Pseudotype MicroNeutralisation or pMN Assay) (FIG. 40):

Panel A (left) Antibody neutralisation of SARS-CoV-2 28 days after 1 dose.

Similar to RBD antibody responses, neutralising antibodies to SARS-CoV-2 were identified. In all groups 28 days following the first immunisation. The top left panel (T2_8 at 400 μg sc) had low level responses compared to the same vaccine candidate (T2_8 at 400 μg DNA) administered ID by the Tropis Pharmajet device, which was the strongest of all the groups. T2_17 ID by PharmaJet delivery, showed lower but significant responses to SARS-CoV-2.

Panel B (right) Antibody neutralisation of SARS 28 days after 1 dose.

The same 28 day serum samples at serial dilutions were tested for neutralising to SARS pseudotyped viruses. At this time point, after 1 administration, responses were absent in the T2_8 groups (top left and bottom right of panel B (right).

The pan-Sarbeco T2_17 groups respond at low and variable levels after 1 dose of vaccine, again with the best but weak response in the highest dose group (400 μg) (bottom row left in panel B)

Groups 1 to 3, Comparison of Virus Neutralisation Responses after First to Second Immunisation (FIG. 41):

Panel A (left SARS-CoV-2) Comparing bleeds 2 (pre) and 3 (post) second immunisation (boost)

There was significant boost effect with increased neutralising responses to SARS-CoV-2 in all groups, though not all animals in group 1 (T2_8 at 400 μg) administered subcutaneously. Groups 2 and 3, middle and lower rows of panel A, left, were more uniform and comparably boosted neutralising titres to SARS-CoV-2.

Panel B (right SARS) Comparing bleeds 2 (pre) and 3 (post) second immunisation (boost).

There was weak and variable boost effect in 5 of 8 animals to SARS in group 1 (T2_8 at 400 μg). Groups 2 and 3, middle and lower rows of panel A, left, were uniform and comparably strongly boosted with significant neutralising titres to SARS.

Groups 4, 5 and 6. Comparison of Virus Neutralisation Responses after First to Second Immunisation (FIG. 42):

Panel A (left SARS-CoV-2) Comparing bleeds 2 (pre) and 3 (post) second immunisation (boost).

Comparing the left hand column of groups 4, 5 and 6, there was significant boost effect with increased neutralising responses to SARS-CoV-2 in Group 4 200 μg T_17 Tropis, group 5 400 μg T_17 Tropis, and the SARS-CoV-2 specific 400 μg T2_8 also delivered by Tropis.

Panel B (right SARS) Comparing bleeds 2 (pre) and 3 (post) second immunisation (boost).

Comparing the left to the right hand column of groups 4, 5 and 6, there was clear boost effect with increased neutralising responses to SARS in all 3 groups, but most significantly in the two T2_17 immunised groups (4 and 5, upper right hand graphs) that received 200 μg (top row panel B), and 400 μg of T2_17 (middle row panel B), with a possible dose effect in the 400 μg dose. In contrast, the 400 μg T2_8 group was boosted to a much lower and variable effect.

### Neutralisation of Variants of Concern (FIG. 43);

Selected high, middle and low neutralising antibody responders from T2_8 and T2_17 guinea pig groups were tested for pseudotype based viral neutralisation of the original Wuhan strain (control), as well as variants of concern (VOC) lineages B1.248 (Brazil P1 lineage) and B1.351 (South Africa). Both these VOCs contain the E484K mutation that confers resistance to current vaccines in use (AstraZeneca, Pfizer, Moderna). High responding T2_8 guinea pig (8 and 11) antisera do not neutralise the VOCs, whereas high responders from the T2_17 group (31 and 34) still neutralise strongly.

### EXAMPLE 34

Nucleic Acid Sequences Encoding COV_S_T2_13-20
>COV_S_T2_13 encoding nucleic acid
(SEQ ID NO: 78)
AGAGTGGCCCCTACCAAAGAAGTCGTGCGGTTCCCCAACATCACCAATCTGTGCCCTTTC
GGCGAGGTGTTCAACGCCACCAGATTTCCCTCTGTGTACGCCTGGGAGAGAAAGCGGATC
AGCAACTGCGTGGCCGACTACAGCGTGCTGTACAACAGCACCAGCTTCAGCACCTTCAAG
TGCTACGGCGTGTCACCCACCAAGCTGAACGACCTGTGCTTCACCAACGTGTACGCCGAC
AGCTTCGTGATCAGAGGCGACGAAGTGCGGCAGATTGCCCCTGGACAAACAGGCGTGATC
GCCGATTACAACTACAAGCTGCCCGACGACTTCACCGGCTGTGTGATCGCCTGGAACACC
AACAACCTGGACAGCACCACCGGCGGCAACTACAACTACCTGTACAGAAGCCTGCGGAAG
TCTAAGCTGAAGCCCTTCGAGCGGGACATCAGCAGCGACATCTATAGCCCTGGCGGCAAG
CCTTGTTCTGGCGTGGAAGGCTTCAACTGCTACTACCCTCTGCGGAGCTACGGCTTCTTC
CCCACAAATGGCGTGGGCTACCAGCCTTACAGAGTGGTGGTCCTGAGCTTCGAGCTGCTG
AATGCCCCTGCCACAGTGTGTGGCCCTAAGCTGTCTACCGAC
>COV_S_T2_14 encoding nucleic acid
(SEQ ID NO: 79)
AGAGTGGCCCCTACCAAAGAAGTCGTGCGGTTCCCCAACATCACCAATCTGTGCCCTTTC
GGCGAGGTGTTCAACGCCACCAAGTTTCCCTCTGTGTACGCCTGGGAGCGCAAAAAGATC
AGCAACTGCGTGGCCGACTACAGCGTGCTGTACAACAGCACCAGCTTCAGCACCTTCAAG
TGCTACGGCGTGTCACCCACCAAGCTGAACGACCTGTGCTTCACCAACGTGTACGCCGAC
AGCTTCGTGATCAGAGGCGACGAAGTGCGGCAGATTGCCCCTGGACAAACAGGCGTGATC
GCCGATTACAACTACAAGCTGCCCGACGACTTCACCGGCTGTGTGATCGCCTGGAACACC
AACAACATCGACAGCACCACCGGCGGCAACTACAACTACCTGTACAGAAGCCTGCGGAAG
TCTAAGCTGAAGCCCTTCGAGCGGGACATCAGCAGCGACATCTATAGCCCTGGCGGCAAG
CCTTGTTCTGGCGTGGAAGGCTTCAACTGCTACTACCCTCTGCGGAGCTACGGCTTCTTC
CCCACAAATGGCGTGGGCTACCAGCCTTACAGAGTGGTGGTCCTGAGCTTCGAGCTGCTG
AATGCCCCTGCCACAGTGTGTGGCCCTAAGCTGTCTACCGAC
>COV_S_T2_15 encoding nucleic acid
(SEQ ID NO: 80)
AGAGTGGCCCCTACCAAAGAAGTCGTGCGGTTCCCCAACATCACCAATCTGTGCCCTTTC
GGCGAGGTGTTCAACGCCACCAGATTTCCCTCTGTGTACGCCTGGGAGAGAAAGCGGATC
AGCAACTGCGTGGCCGACTACAGCGTGCTGTACAACAGCACCTTCTTCAGCACCTTTAAG
TGCTACGGCGTGTCACCCACCAAGCTGAACGACCTGTGCTTCAGCAACGTGTACGCCGAC
AGCTTCGTGATCAGAGGCGACGAAGTGCGGCAGATTGCCCCTGGACAAACAGGCGTGATC
GCCGATTACAACTACAAGCTGCCCGACGACTTCATGGGCTGTGTGATCGCCTGGAACACC
AACAACCTGGACAGCACCACCGGCGGCAACTACAACTACCTGTACAGAAGCCTGCGGAAG
TCTAAGCTGAAGCCCTTCGAGCGGGACATCAGCAGCGACATCTATAGCCCTGGCGGCAAG
CCTTGTTCTGGCGTGGAAGGCTTCAACTGCTACTACCCTCTGCGGAGCTACGGCTTCTTC
CCCACAAATGGCGTGGGCTACCAGCCTTACAGAGTGGTGGTCCTGAGCTTCGAGCTGCTG
AATGCCCCTGCCACAGTGTGTGGCCCTAAGCTGTCTACCGAC
>COV_S_T2_16 encoding nucleic acid
(SEQ ID NO: 81)
AGAGTGGCCCCTACCAAAGAAGTCGTGCGGTTCCCCAACATCACCAATCTGTGCCCTTTC
GGCGAGGTGTTCAACGCCACCAGATTTCCCTCTGTGTACGCCTGGGAGAGAAAGCGGATC
AGCAACTGCGTGGCCGACTACAGCGTGCTGTACAACAGCACCAGCTTCAGCACCTTCAAG
TGCTACGGCGTGTCACCCACCAAGCTGAACGACCTGTGCTTCACCAACGTGTACGCCGAC
AGCTTCGTGATCAGAGGCGACGAAGTGCGGCAGATTGCCCCTGGACAGACAGGCAAGATC
GCCGATTACAACTACAAGCTGCCCGACGACTTCACCGGCTGTGTGATCGCCTGGAACACC
AACAACCTGGACAGCACCACCGGCGGCAACTACAACTACCTGTACCGGCTGTTCCGGAAG
TCCAACCTGAAGCCTTTCGAGCGGGACATCAGCAGCGACATCTATCAGGCCGGCAGCACA
CCTTGTTCTGGCGTGGAAGGCTTCAACTGCTACTTCCCACTGCAAAGCTACGGCTTCCAG
CCTACCAACGGCGTGGGCTACCAGCCTTATAGAGTGGTGGTCCTGAGCTTCGAGCTGCTG
AATGCCCCTGCCACAGTGTGTGGCCCTAAGCTGTCTACCGAC
>COV_S_T2_17 encoding nucleic acid
(SEQ ID NO: 82)
AGAGTGGCCCCTACCAAAGAAGTCGTGCGGTTCCCCAACATCACCAATCTGTGCCCTTTC
GGCGAGGTGTTCAACGCCACCAAGTTTCCCTCTGTGTACGCCTGGGAGCGCAAAAAGATC
AGCAACTGCGTGGCCGACTACAGCGTGCTGTACAACAGCACCAGCTTCAGCACCTTCAAG
TGCTACGGCGTGTCACCCACCAAGCTGAACGACCTGTGCTTCACCAACGTGTACGCCGAC
AGCTTCGTGATCAGAGGCGACGAAGTGCGGCAGATTGCCCCTGGACAAACAGGCGTGATC
GCCGATTACAACTACAAGCTGCCCGACGACTTCACCGGCTGTGTGATCGCCTGGAACACC
AACAACATCGACAGCACCACCGGCGGCAACTACAACTACCTGTACAGAAGCCTGCGGAAG
TCTAAGCTGAAGCCCTTCGAGCGGGACATCAGCAGCGACATCTATAGCCCTGGCGGCAAG
CCTTGTTCTGGCGTGGAAGGCTTCAACTGCTACTACCCTCTGCGGAGCTACGGCTTCTTC
CCCACAAATGGCACAGGCTACCAGCCTTACAGAGTGGTGGTCCTGAGCTTCGAGCTGCTG
AATGCCCCTGCCACAGTGTGTGGCCCTAAGCTGTCTACCGAC
>COV_S_T2_18 encoding nucleic acid
(SEQ ID NO: 83)
AGAGTGGCCCCTACCAAAGAAGTCGTGCGGTTCCCCAACATCACCAATCTGTGCCCTTTC
GGCGAGGTGTTCAACGCCACCAGATTTCCCTCTGTGTACGCCTGGGAGAGAAAGCGGATC
AGCAACTGCGTGGCCGACTACAGCGTGCTGTACAACAGCACCTTCTTCAGCACCTTTAAG
TGCTACGGCGTGTCACCCACCAAGCTGAACGACCTGTGCTTCAGCAACGTGTACGCCGAC
AGCTTCGTGATCAGAGGCGACGAAGTGCGGCAGATTGCCCCTGGACAAACAGGCGTGATC
GCCGATTACAACTACAAGCTGCCCGACGACTTCATGGGCTGTGTGATCGCCTGGAACACC
AACAACCTGGACAGCACCACCGGCGGCAACTACAACTACCTGTACAGAAGCCTGCGGAAG
TCTAAGCTGAAGCCCTTCGAGCGGGACATCAGCAGCGACATCTATAGCCCTGGCGGCAAG
CCTTGTTCTGGCGTGGAAGGCTTCAACTGCTACTACCCTCTGCGGAGCTACGGCTTCTTC
CCCACAAATGGCACAGGCTACCAGCCTTACAGAGTGGTGGTCCTGAGCTTCGAGCTGCTG
AATGCCCCTGCCACAGTGTGTGGCCCTAAGCTGTCTACCGAC
>COV_S_T2_19 encoding nucleic acid
(SEQ ID NO: 84)
AGAGTGGCCCCTACCAAAGAAGTCGTGCGGTTCCCCAACATCACCAATCTGTGCCCTTTC
GGCGAGGTGTTCAACGCCACCAGATTTCCCTCTGTGTACGCCTGGGAGAGAAAGCGGATC
AGCAACTGCGTGGCCGACTACAGCGTGCTGTACAACAGCACCAGCTTCAGCACCTTCAAG
TGCTACGGCGTGTCACCCACCAAGCTGAACGACCTGTGCTTCACCAACGTGTACGCCGAC
AGCTTCGTGATCAGAGGCGACGAAGTGCGGCAGATTGCCCCTGGACAAACAGGCGTGATC
GCCGATTACAACTACAAGCTGCCCGACGACTTCACCGGCTGTGTGATCGCCTGGAACACC
AACAACCTGGACAGCACCACCGGCGGCAACTACAACTACCTGTACAGAAGCCTGCGGAAG
TCTAAGCTGAAGCCCTTCGAGCGGGACATCAGCAGCGACATCTATAGCCCTGGCGGCAAG
CCTTGTTCTGGCGTGGAAGGCTTCAACTGCTACTACCCTCTGCGGAGCTACGGCTTCTTC
CCCACAAATGGCGTGGGCTACCAGCCTTACAGAGTGGTGGTCCTGAGCTTCGAGCTGCTG
AATGCCCCTGCCACAGTGTGTGGCCCTAAGCTGTCTACAGATGGCGGCGGAGGATCTGGC
GGAGGTGGAAGCGGAGGCGGAGGAAGCGGTGGCGGCGGATCTAAATCTTCTATCGCCAG
CTTCTTCTTCATCATCGGCCTGATTATCGGCCTGTTCCTGGTGCTGAGAGTGGGCATCCAC
CTGTGCATCAAGCTGAAACACACCAAGAAGCGGCAAATCTACACCGACATCGAGATGAAC
CGGCTGGGCAAA
>COV_S_T2_20 encoding nucleic acid
(SEQ ID NO: 85)
AGAGTGGCCCCTACCAAAGAAGTCGTGCGGTTCCCCAACATCACCAATCTGTGCCCTTTC
GGCGAGGTGTTCAACGCCACCAAGTTTCCCTCTGTGTACGCCTGGGAGCGCAAAAAGATC
AGCAACTGCGTGGCCGACTACAGCGTGCTGTACAACAGCACCAGCTTCAGCACCTTCAAG
TGCTACGGCGTGTCACCCACCAAGCTGAACGACCTGTGCTTCACCAACGTGTACGCCGAC
AGCTTCGTGATCAGAGGCGACGAAGTGCGGCAGATTGCCCCTGGACAAACAGGCGTGATC
GCCGATTACAACTACAAGCTGCCCGACGACTTCACCGGCTGTGTGATCGCCTGGAACACC
AACAACATCGACAGCACCACCGGCGGCAACTACAACTACCTGTACAGAAGCCTGCGGAAG
TCTAAGCTGAAGCCCTTCGAGCGGGACATCAGCAGCGACATCTATAGCCCTGGCGGCAAG
CCTTGTTCTGGCGTGGAAGGCTTCAACTGCTACTACCCTCTGCGGAGCTACGGCTTCTTC
CCCACAAATGGCACAGGCTACCAGCCTTACAGAGTGGTGGTCCTGAGCTTCGAGCTGCTG
AATGCCCCTGCCACAGTGTGTGGCCCTAAGCTGTCTACAGATGGCGGCGGAGGATCTGGC
GGAGGTGGAAGCGGAGGCGGAGGAAGCGGTGGCGGCGGATCTAAATCTTCTATCGCCAG
CTTCTTCTTCATCATCGGCCTGATTATCGGCCTGTTCCTGGTGCTGAGAGTGGGCATCCAC
CTGTGCATCAAGCTGAAACACACCAAGAAGCGGCAAATCTACACCGACATCGAGATGAAC
CGGCTGGGCAAA

### EXAMPLE 35

Gene Delivery of a Structurally Engineered Coronavirus Vaccine Candidate Elicits Pan-Sarbecovirus Neutralisation and Protects from Delta Variant Challenge

### SUMMARY

Of the Coronaviruses that have caused zoonotic spill-overs in past two decades, the ACE-2 receptor-using sarbeco Coronaviruses have caused the most severe human epidemics of highest morbidity and mortality. The COVID-19 pandemic and emerging variants have highlighted the need for vaccines capable of providing broader protection. Here we report on an engineered antigen structure of conserved receptor binding domain (RBD) epitopes, immune selected to protect against diverse sarbecoviruses. From an array of phylogenetically informed antigen structures displaying different broad neutralising epitopes, synthetic genes expressing these were selected based on broad immune responses in mice. Immunogenicity of the lead vaccine antigen was confirmed in Guinea pigs using needleless intradermal immunisation. The broad neutralising immune profile against SARS-CoV-1, SARS-CoV-2, WIV16, and RaTG13 was further confirmed in Rabbits with GMP manufactured DNA. Notably sera from immunised Rabbits showed potent antibody responses against Beta, Gamma, and Delta variants of concern (VOC) and protected vaccinated mice from Delta challenge. These findings demonstrate the potential of a novel phylogenetically informed, structurally engineered vaccine antigen pipeline for the in vivo selection of nucleic acid-based immunogens, and a pan-sarbecovirus vaccine candidate capable of generating broad neutralising antibodies across the sarbecoviruses.

### Results

Amongst the coronaviruses of the greatest pandemic risk are the angiotensin-converting enzyme 2 (ACE-2) binding viruses of sarbecoviruses sub-genus of the genus β-Coronaviruses resident in diverse bat species^{1.2}. Over the last two decades, two ACE-2 binding sarbecoviruses have spilled over into human population causing the SARS epidemic in 2002/2003 and the current on-going SARS-CoV-2 pandemic. Bats are the reservoir of a large number of SARS-CoV-like ACE-2 binding sarbecoviruses which pose a constant threat for future spill-over into human population, and potentially new epidemics^{3.4}. In addition to emergence of new ACE-2 binding viruses from zoonotic reservoirs, another concern is emergence of variants of these viruses capable of escaping vaccine-induced immunity, a constant concern in the current on-going pandemic. As human infections increase globally during the current pandemic, the virus has continued to accrue mutations, most significantly in the spike protein⁵. An accumulating number of variants of concerns (VOCs) have implications for increased transmission and escape from natural and vaccine immunity⁶⁻⁹. The N501Y asparagine to tyrosine in the receptor binding domain (RBD) of the spike protein is a common feature of VOCs and is associated with increased affinity of the viral particle to the ACE-2 receptor and subsequent increase in transmission¹⁰. Two of the variants have K417N/T and E484K mutation in the RBD and is reported to escape immune responses generated by most approved vaccines^{7,8}. The delta VOC¹¹ is the most contagious variants reported to date, with L452R and T478K mutation in the RBD. Notably, the majority of these mutations reported in VOCs are in or around the region in RBD that interacts with ACE-2 as well as one of the regions that induce highly potent neutralising antibodies^{12,13}. The continued emergence of these VOCs during the on-going COVID-19 pandemic, and the constant threat of new zoonotic spill overs of coronaviruses from animals to humans, highlights the need for next generation vaccines with broader protection from ACE-2 binding sarbecoviruses as well as the emerging VOCs. To increase the coverage to all ACE-2 receptor using viruses of the sarbecovirus sub-genus of β-coronaviruses, a structure-based, RBD subunit-based vaccine strategy was employed comparing all the known human and animal reservoir sarbecoviruses. This design was further used as backbone for designing both epitope optimised and immune re-focussed designs using available structural data from a number of high-quality structural data is available for spike protein in complex with monoclonal antibodies, specifically those targeting the ACE-2 receptor binding domain (RBD), such as S30914 and CR302215 that bind both SARS-CoV-1 and SARS-CoV-2. The nucleic acid sequence of these in silico designed vaccine antigens were optimised for expression in humans and synthetic genes expressing each unique antigen structure was shuttled in an expression cassette for in vitro and in vivo screens to select the optimal antigen as the vaccine candidate for nucleic acid vaccine delivery.

Sequences of spike protein of viruses belonging to the sarbecovirus lineage were compiled from NCBI virus database¹⁶ and pruned. The phylogenetic tree of these sequences is represented in FIG. 44A. Two distinct clades are observed in the tree, separating those in clade 1 which do not interact with ACE-2 receptor^{1,17} from those in clade 2 which do. Clade 1 viruses share many of the sequence feature of the members of clade 2 but possess deletions around the ACE-2 binding region (FIG. 46). An optimised core sequence (T2_13) was designed, such that each amino acid position in this sequence was optimised to be phylogenetically closer to all the sarbecoviruses represented in the phylogenetic tree in FIG. 44A. To further understand the importance of amino-acid composition of epitopes in generating antibody responses, we further modified T2_13 to display the epitopes of SARS-CoV-1 for monoclonal antibodies-S309¹⁴ (T2_14), and CR3022¹⁵ (T2_15) and of SARS-CoV-2 for monoclonal antibody-B38¹² (T2_16). The sequence of epitopes for monoclonal antibodies-S309¹⁴, and CR3022¹⁵ are highly conserved across the sequences considered in this study while the sequence of epitopes for monoclonal antibody-B38¹² is highly divergent (FIG. 44B). We further modified the epitope region for monoclonal antibody-B38¹² by introducing a glycosylation site on the backbone of T2_14 (T2_17) and T2_15 (T2_16). This was done to mask the divergent epitope region and enhance the presentation of the conserved epitopes to the immune system. The masking of epitopes by introducing glycans has been exploited by many viruses such as Hepatitis C Virus¹⁸ and Lassa virus¹⁹ to escape natural immunity. To compare the immunogenicity of soluble and membrane bound RBD subunit-based vaccine, membrane bound forms of T2_13 and T2_17 (T2_19 and T2_20 respectively) were generated. The structural stability of these designs was evaluated in-silico using FOLDX²⁰ algorithm. Structural models of these vaccine antigens are represented in FIG. 44C.

In vivo screening in BALB/c mice was performed by immunising different the lead antigen designs and assaying for cross reactive antibodies against different sarbeco Spike proteins by FACS cell-surface display (FIG. 44D). Sera taken two weeks following the second immunisation with antigen designs (T2_13 through to T2_20), demonstrated the binding profile of the vaccine candidates for different spike proteins (FIG. 44E). As expected, sera from SARS-CoV-1 RBD immunised mice bound strongly to both homologous SARS-CoV-1 spike protein and closely related WIV16 spike in comparison to other vaccine designs, while sera from SARS-CoV-2 RBD immunised mice binds homologous SARS-CoV-2 spike protein and closely related RaTG13 spike protein in the similar range of other vaccine designs. Sera from SARS-CoV-1 immunised mice show binding to SARS-CoV-2 spike, though significantly less than sera from SARS-CoV-2 RBD immunised mice (Mann-Whitney U test, p-value=0.02). Across the four spike proteins, no significant differences in binding were observed for sera from mice immunised with T2_13 and sera from mice immunised with SARS-CoV-2 RBD (Mann-Whitney U test, all p-values >0.05), demonstrating that epitopes in this design is biased towards SARS-CoV-2 RBD. For the T2_16 design, in which the epitope region for mAb B38 was matched identical to SARS-CoV-2, binding to SARS-CoV-1, WIV16, and RaTG13 declined in comparison to T2_13 (Mann-Whitney U test, p-value <0.05) without statistical changes in binding to SARS-CoV-2. This observation is suggestive of immunodominance of this region in comparison to other sites. Matching of the epitopes of S309 and CR3022 to SARS-CoV-1 (T2_14 and T2_15), enhanced the binding to SARS-CoV-1 (Mann-Whitney U test, p-value <0.05) but not to other spike proteins. Introduction of glycosylation site in design T2_17 significantly enhanced the binding of SARS-CoV-1 and RaTG13 (Mann-Whitney U test, p-value <0.01) in comparison to T2_14, but no difference is observed in T2_18 in comparison to T2_15. There was no difference between trans-membrane and non-trans-membrane bound designs. Elicitation of cross-binding antibodies by T2_17 was further confirmed by ELISA with SARS-CoV-1 RBD and SARS-CoV-2 RBD (FIG. 44F) revealing robust antibody responses to both SARS-CoV-1 and SARS-CoV-2 within two weeks of the second immunisation. While the T2_17 antigen elicited stronger responses against SARS-CoV-1, it was lower than those induced by the homologous SARS-CoV-1 antigen, but significantly higher than SARS-CoV-2. Against SARS-CoV-2, all the three antigens—SARS-CoV-1 RBD, SARS-CoV-2 RBD, and T2_17 generated similar binding antibody responses. Given the breadth of antibody responses induced by the T2_17 antigen, we asked if this antigen could boost and broaden the efficacy of current licensed vaccines against SARS-CoV-2 VOCs. To address this we used homozygous K18 hACE2 transgenic mice and immunised them with 1.4 e9 vp of commercially available AZD1222 (ChAdOx1 nCOV-19) and 4 weeks later re-boosted with either T2_17, SARS-CoV-2 RBD or the AZD1222 vaccine (FIG. 44G), while the control group received only PBS with each immunisation. Eight weeks post boost, all groups of mice were challenged with either with a January 2020 isolate of SARS-CoV-2 or the more recent Delta variant of SARS-CoV-2 (FIG. 44H). Binding antibodies to both SARS-CoV-1 and SARS-CoV-2 were observed four weeks after immunisation and boosting by either AZD1222 or T2_17 or SARS-CoV-2_RBD which further increased the antibody titres for both SARS-CoV and SARS-CoV-2 (FIG. 47A). Significant difference in antibody titres to SARS-CoV-2 were observed four weeks after boosting with T2_17 and SARS-CoV-2_RBD in comparison to boosting by AZD1222 (FIG. 44I).

Neutralising antibodies for SARS-CoV-2 and the Delta VOC were detected four weeks post boost for all the groups, except the control group prior to challenge (FIG. 47B). T2_17 neutralised the Delta variant significantly better than the sera from mice boosted with AZD1222 (FIG. 44J). Mice from all the groups, except controls, survived and continued to gain weight following challenge with either the vaccine strain or Delta variant (FIG. 44K).

To determine the optimal dose for the best breadth of antibody neutralisation responses in outbred animals, Guinea pigs were immunised with different doses of T2_17 DNA using a CE approved, and clinically validated Pharmajet Tropis needleless, intradermal delivery device ensure standardised intradermal delivery (FIG. 45A). As a control we used a C-terminal glycosylation modified SARS-CoV-2 RBD (SARS2_RBD_P521N) (FIG. 45B) which we had previously evaluated in BALB/c mice (FIG. 48). Generation of binding, and neutralising antibodies to both SARS-CoV-1 and SARS-CoV-2 was confirmed using lenti-pseudovirus neutralisation expressing full-length spike proteins of SARS-CoV-1, and SARS-CoV-2. While both T2_17 and SARS2_RBD_P521N generated binding antibodies against both SARS-CoV-1 and SARS-CoV-2 (FIG. 45C) after one immunisation, T2_17 elicited significantly higher antibodies than SARS2_RBD_P521N to SARS-CoV-1 and comparable antibodies against SARS-CoV-2. Higher binding antibodies were detected for T2_17 to SARS-CoV-1 in comparison to SARS2_RBD_P521N after two immunisations while the responses were comparable for SARS-CoV-2. After three immunisations SARS2_RBD_P521N had developed a bias response to SARS-CoV-2, while T2_17 had higher responses to SARS-CoV. Neutralising antibodies were detected for SARS-CoV-2 after first immunisation while neutralising responses to SARS-CoV-1 became significant following two immunisations, though more potent for T2_17 than SARS2_RBD_P521N (FIG. 45D). Better binding and neutralising response by SARS2_RBD_P521N were expected as it differs from SARS-CoV-2 by only one amino acid. To further confirm, whether T2_17 vaccine design generates broader responses, we compared sera induced by SARS2_RBD_P521N, 30 days post 3rd immunisation for neutralisation against SARS-CoV-1, WIV16, RaTG13, and SARS-CoV-2. Statistically significantly higher neutralising antibodies were generated by T2_17 against SARS-CoV-1, WIV16, and RaTG13 (FIG. 45E). To further confirm T2_17 anti-sera could abrogate HuACE2 receptor binding, we performed an ELISA based competition assay (FIG. 45F) demonstrating T2_17 and SARS2_RBD_P521N anti-sera abrogated binding to ACE-2 receptor and are comparable to the international standard of pooled convalescent COVID-19 patient sera. These findings demonstrated important proof-of-concept of T2_17 as a single gene delivered, structurally engineered antigen capable of eliciting broad pan-sarbeco Coronavirus neutralising antibodies.

Prior to clinical trials in humans, a GMP lot of pEVAC T2_17 was manufactured and evaluated for safety and immunogenicity in Rabbits using the same gene delivery device to ensure uniform intradermal administration (FIG. 45G). After one immunisation, binding antibodies to SARS-CoV-1 and SARS-CoV-2 were elicited (FIG. 45H), increasing on subsequent immunisations until a plateau was reached by the fourth immunisation. Robust neutralising antibodies were observed 2 weeks following the third immunisation (FIG. 45I) revealing broad neutralising antibody responses against the SARS-CoV-1, SARS-CoV-2, Beta, Gamma, and Delta VOCs as well as the bat sarbecoviruses-WIV16, and RaTG13 elicited by gene delivery of the engineered T2_17 pan-Sarbeco vaccine antigen candidate (FIG. 45J). It is important to note that sera from animals immunised with T2_17 had at least a fold IC₅₀ greater neutralisation than the WHO reference standard for SARS-CoV-2 vaccine evaluation.

Emergence of two ACE-2 binding sarbecoviruses in past two decades highlights the urgent need for vaccines that can provide broad protection from SARS-CoV-2 VOCs as well as to the ACE-2 receptor using sarbecoviruses that threaten to spill-over from zoonotic animal reservoirs. Here, we describe a structural informatics platform pipeline for generating broadly reactive vaccine antigens expressed as synthetic genes that can be delivered by nucleic acid delivery. As proof of concept, lead vaccine antigen candidates were immune selected to elicit a broad neutralisation profile demonstrated in 3 species against SARS-CoV-1, WIV16, RaTG13, SARS-CoV-2 and it's VOCs. Further broadening of vaccine protection is being expanded by the use of multiple digitally immune optimised antigens selected to immunologically recruit additional T and B effector responses for maximising breadth and depth of immunity to pre-emergent Coronaviruses or Influenza viruses, while reducing the potential for immune escape.

### Figure Legends

### FIG. 44—In-Silico Design and In-Vivo Selection of Vaccine Antigen Candidate.

A. Phylogenetic tree generated for sarbecoviruses using protein sequence of receptor binding domain (RBD) of the spike protein. The tree was generated using IQ-Tree¹⁷. Human viruses are represented in green, palm civet viruses in pink and bat viruses in dark grey. The distinct two clades are coloured in red (non-ACE-2 binding) and blue (ACE-2 binding). B. Structural models of RBD with epitope regions highlighted as spheres. The backbone of RBD is coloured according to the CONSURF score calculated using the alignment used for construction of phylogenetic tree. The figure was generated and rendered using PyMol using PDB id 6wps¹⁴, 6w41¹⁵, and 7bz5¹⁹. C. Structural representation of the different vaccine designs used in this study. The epitopes that were modified to match the wild-type SARS-CoV-1 (coloured orange) and wild-type SARS-CoV-2 (coloured grey) are represented in spheres. Further glycosylation site modification is represented in green sphere. D. Immunisation and bleed schedule of BALB/c mice. Mice were immunised at interval of 30 days and bled every 15 days. E. FACS binding data for different vaccine designs. Sera from mice immunised with these vaccine antigens were screened for binding to SARS-CoV-1, SARS-CoV-2, WIV16, and RaTG13 spike proteins. The X-axis represents the mean fluorescence intensity (MFI), and the Y-axis represents all the vaccine designs considered for screening. F. Elicitation of binding anti-bodies against SARS-CoV-1 and SARS-CoV-2 by T2_17 was confirmed using ELISA, with SARS-CoV-1 and SARS-CoV-2 RBD as control vaccine design. T2_17 generated cross-binding antibodies. The X-axis represents the vaccine designs, and the Y-axis represents the area under the curve (AUC) for ELISA binding curves. G. Immunisation, bleed, and challenge schedule of K18 hACE2 mice. H. K18 hACE2 mice were primed with AZD1222 vaccine and then boosted with either AZD1222, T2_17, or SARS2_RBD after four weeks. The mice were challenged after 8 weeks with either Victoria strain of SARS-CoV-2 or the delta variant. I. Elicitation of binding anti-bodies against SARS-CoV-1 and SARS-CoV-2 before challenge was confirmed using ELISA using K18 hACE2 mice sera 4 weeks post boost (bleed4). Boost by T2_17, and SARS2_RBD significantly increased the binding antibody titres in comparison to boost by AZD1222. The X-axis represents the vaccine designs, and the Y-axis represents the area under the curve (AUC) for ELISA binding curves. J. Neutralisation of SARS-CoV-1, SARS-CoV-2, and delta variant of SARS-CoV-2 by K18 hACE2 mice sera 4 weeks post boost (bleed4). Sera of mice boosted with T2_17 significantly neutralised the Delta variant (B.1.617.2) in comparison to those boosted by AZD1222. The X-axis represents the bleed number, and the Y-axis represents the log₁₀IC₅₀ values for neutralisation curves. K. Weight loss profile of K18 hACE2 mice following challenge by the Victoria strain and the delta variant. All the mice, except naïve were protected. Mann-Whitney U test is used as statistical significance test in all the plots (p-value: *≤0.05, **<0.01, ***≤0.001).

### FIG. 45—Immunogenicity Studies in Guinea Pigs and Rabbits

A. Immunisation and bleed schedule of Guinea pigs. Guinea pigs were immunised using DNA delivered intradermally (i.d) by the Tropis ParmaJet device at 28 day intervals of 30 days and bled every 15 days. B. Structure models of the vaccine designs used for the study in Guinea pigs. The glycosylation site and the modified epitope are represented as green and orange spheres respectively. C. Elicitation of binding anti-bodies against SARS-CoV-1 and SARS-CoV-2 by T2_17 and SARS2_RBD_P521N was confirmed using ELISA. T2_17 and SARS2_RBD_P521N generated cross-binding antibodies after one immunisation. The pre-bleed (Bleed 0) is considered as the control for non-specific binding. The X-axis represents the bleed number, and the Y-axis represents the area under the curve (AUC) for ELISA binding curve. D. Neutralisation by Guinea pig sera immunised with T2_17 and SARS2_RBD_P521N. Both T2_17 and SARS2_RBD_P521N generated neutralising antibodies against SARS-CoV-1 and SARS-CoV-2. The X-axis represents the bleed number, and the Y-axis represents the log₁₀IC₅₀ values for neutralisation curves. E. Broad-neutralisation of SARS-CoV-1, WIV16, RaTG13, and SARS-CoV-2 by T2_17 in comparison to SARS2_RBD_P521N. Sera post 30 days after three immunisation (bleed 6) was used for comparison. F. ACE-2 competition ELISA. Guinea pigs sera immunised with T2_17 and SARS2_RBD_P521N effectively abrogated the interaction of SARS-CoV-2 RBD with ACE-2 receptor and were more potent in abrogating the interaction that the NIBSC standard (20/162). G. Immunisation and bleed schedule of Rabbits. Rabbits were immunised at interval of 15 days and bled every 15 days. H. Elicitation of binding anti-bodies against SARS-CoV-, 1 and SARS-CoV-2 by T2_17 was confirmed using ELISA. T2_17 generated cross-binding antibodies after one immunisation. The X-axis represents the bleed number, and the Y-axis represents the area under the curve (AUC) for ELISA binding curve. I. Neutralisation by Rabbit sera immunised with T2_17. T2_17 generated neutralising antibodies against SARS-CoV-1 and SARS-CoV-2. The X-axis represents the bleed number, and the Y-axis represents the log₁₀IC₅₀ values for neutralisation curves. J. Broad-neutralisation of SARS-CoV-1, WIV16, RaTG13, SARS-CoV-2, SARS-CoV-2 Beta, SARS-CoV-2 Gamma, and SARS-CoV-2 Delta by T2_17. Sera post 15 days after four immunisation (bleed 4) was used for comparison. NISBSC standard for SARS-CoV-2 and SARS-CoV-1 antiserum are used as reference. Mann-Whitney U demonstrated statistical significance (p-value: *≤0.05, **<0.01, ***≤0.001).

### REFERENCES

1. Liu, K. et al. Cross-species recognition of SARS-CoV-2 to bat ACE2. Proc. Natl. Acad. Sci. U.S.A 118, (2021).
2. Olival, K. J. et al. Possibility for reverse zoonotic transmission of SARS-CoV-2 to free-ranging wildlife: A case study of bats. PLOS Pathog. 16, e1008758 (2020).
3. Hu, B. et al. Discovery of a rich gene pool of bat SARS-related coronaviruses provides new insights into the origin of SARS coronavirus. PLOS Pathog. 13, e1006698 (2017).
4. Menachery, V. D. et al. A SARS-like cluster of circulating bat coronaviruses shows potential for human emergence. Nat. Med. 21, 1508-1513 (2015).
5. Vilar, S. & Isom, D. G. One Year of SARS-CoV-2: How Much Has the Virus Changed? Biology 10, (2021).
6. Horspool, A. M. et al. SARS-CoV-2 B.1.1.7 and B.1.351 variants of concern induce lethal disease in K18-hACE2 transgenic mice despite convalescent plasma therapy. BioRxiv Prepr. Serv. Biol. (2021) doi: 10.1101/2021.05.05.442784.
7. Planas, D. et al. Sensitivity of infectious SARS-CoV-2 B. 1.1.7 and B.1.351 variants to neutralizing antibodies. Nat. Med. 27, 917-924 (2021).
8. Wang, P. et al. Increased resistance of SARS-CoV-2 variant P.1 to antibody neutralization. Cell Host Microbe 29, 747-751.e4 (2021).
9. Leung, K., Shum, M. H., Leung, G. M., Lam, T. T. & Wu, J. T. Early transmissibility assessment of the N501Y mutant strains of SARS-CoV-2 in the United Kingdom, October to November 2020. Euro Surveill. Bull. Eur. Sur Mal. Transm. Eur. Commun. Dis. Bull. 26, (2021).
10. Tian, F. et al. Mutation N501Y in RBD of Spike Protein Strengthens the Interaction between COVID-19 and its Receptor ACE2. bioRxiv 2021.02.14.431117 (2021) doi: 10.1101/2021.02.14.431117.
11. Campbell, F. et al. Increased transmissibility and global spread of SARS-CoV-2 variants of concern as at June 2021. Eurosurveillance 26, 2100509 (2021).
12. Wu, Y. et al. A noncompeting pair of human neutralizing antibodies block COVID-19 virus binding to its receptor ACE2. Science 368, 1274-1278 (2020).
13. Hwang, W. C. et al. Structural Basis of Neutralization by a Human Anti-severe Acute Respiratory Syndrome Spike Protein Antibody, 80R. J. Biol. Chem. 281, 34610-34616 (2006).
14. Pinto, D. et al. Cross-neutralization of SARS-CoV-2 by a human monoclonal SARS-CoV antibody. Nature 583, 290-295 (2020).
15. Yuan, M. et al. A highly conserved cryptic epitope in the receptor binding domains of SARS-CoV-2 and SARS-CoV. Science 368, 630-633 (2020).
16. Hatcher, E. L. et al. Virus Variation Resource-improved response to emergent viral outbreaks. Nucleic Acids Res. 45, D482-D490 (2017).
17. Li, W. et al. Animal Origins of the Severe Acute Respiratory Syndrome Coronavirus: Insight from ACE2-S-Protein Interactions. J. Virol. 80, 4211-4219 (2006).
18. Lavie, M., Hanoulle, X. & Dubuisson, J. Glycan Shielding and Modulation of Hepatitis C Virus Neutralizing Antibodies. Front. Immunol. 9, 910 (2018).
19. Watanabe, Y. et al. Structure of the Lassa virus glycan shield provides a model for immunological resistance. Proc. Natl. Acad. Sci. U.S.A 115, 7320-7325 (2018).
20. Schymkowitz, J. et al. The FoldX web server: an online force field. Nucleic Acids Res. 33, W382-W388 (2005).

### Methods

### Phylogenetic Analysis

Protein sequences of spike proteins were downloaded from the NCBI virus database for the sarbecoviruses. Multiple sequence alignment (MSA) was generated using the MUSCLE algorithm¹. The resulting MSA was pruned to the RBD region and used as input for phylogenetic tree reconstruction. The phylogenetic tree was generated using IQTREE algorithm² using protein model with best AIC score. The resultant tree was used for generation of phylogenetically optimised design using HyPhy algorithm³.

### Epitope Identification

Available structural data for Spike protein-antibody complexes for SARS-CoV-1 and SARS-CoV-2 were downloaded from the Protein Databank (PDB)⁴. Structural data were then pruned for antigen-antibody complexes where the epitopes are on the RBD. Amino acid residues of antigen that have at least one atom within 5 Å radii of at least one atom of amino acid of antibody are defined as epitope residues. And epitope regions are defined as contiguous stretches of at least 5 amino acids.

### Molecular Modelling

Structural models were generated for T2_13 using MODELLER algorithm^{5,6}. The structural model with the highest DOPE score⁷ was chosen as the working model for the further molecular modelling. The side chains for the model were further optimised using SCWRL library⁸ and energy minimised using GROMACS package⁹. Structural stability of the COV_S_T2_14-COV_S_T2_18 designs was checked for using POSSCAN and BUILD module of FOLDX algorithm¹⁰ using the optimised structural model of COV_S_T2_13.

### Fluorescence Assisted Cell Sorting (FACS) Assay

HEK293T cells were transfected with an expression plasmid expressing wild-type Spike glycoprotein of each of the four ACE-2 binding sarbecoviruses including SARS-CoV-1, SARS-CoV-2, RaTG13, and WIV16. 48 hours after transfection, cells were transferred into V-bottom 96-well plates (20,000 cells/well). Cells were incubated with sera (diluted at 1:50 in PBS) or anti-human IgG Isotype negative control (Invitrogen 02-7102, diluted 1:500 in PBS) for 30 min, washed with FACS buffer (PBS, 1% FBS, 0.02% Tween 20) and stained with Goat anti-Human IgG (H+L) Alexa Fluor 647 Secondary Antibody (diluted at 20 μg/mL in FACS buffer), for 30 min in the dark. Cells were washed with FACS buffer and samples were run on a Attune NxT Flow Cytometer (Invitrogen) with a high-throughput auto sampler. Dead cells were excluded from the analysis by staining cells with 7-Aminoactinomycin D (7-AAD) and gating 7-AAD negative live cells.

### Enzyme-Linked Immunosorbent Assay (ELISA)

The assays were adapted from those originally described by Amanat and co-workers¹¹. Briefly, Nunc MaxiSorp™ flat-bottom plates were coated with 50 μl per well of 1 μg/ml of RBD from SARS-1 or SARS-2 DPSB (−Ca²⁺/−Mg²⁺) and incubated overnight at 4° C. The next day, the plates were blocked with 3% milk in PBST (0.1% w/v Tween20 in PBS) for 1 hour. After removing the blocking buffer, 50 μl/well of serum samples, diluted in PBST-NFM (1% w/w non-fat milk in PBST) were added to the plates and incubated on a plate shaker for two hours at 20° C. The plates were washed three times with 200 μl of PBST, and 50 μl of HRP-conjugated goat anti-human Ig (H and L chains) (Jackson ImmunoResearch) was added to each well and left to incubate for one hour on a plate shaker for 1 hour. Plates were washed three times with 200 μl of PBST and 50 μl/well of 1-Step Ultra TMB chromogenic substrate (Sigma) were added to the plates and the chemical reaction was stopped three minutes later with 50 μl 2N H₂SO₄. The optical density at a wavelength of 450 nm (OD450) was measured using a Biorad microplate reader. Values from the dilution curved were used to determine the area under the curve.

### Pseudotype-Based Micro-Neutralisation Assay

Pseudotype-based micro-neutralisation assay was performed as described previously¹². Briefly, serial dilutions of serum were incubated with SARS-2/RaTG13/SARS-1/WIV16 spike bearing lentiviral pseudotypes for 1 h at 37° C., 5% CO₂ in 96-well white cell culture plates. 1.5×10⁴ HEK293T/17 transiently expressing human ACE-2 and TMPRSS2 were then added per well and plates incubated for 48 h at 37° C., 5% CO₂ in a humidified incubator. Bright-Glo (Promega) was then added to each well and luminescence read after a five-minute incubation period. Experimental data points were normalised to 100% and 0% neutralisation controls and non-linear regression analysis performed to produce neutralisation curves and IC₅₀ values.

### Vaccination Experiments in Mice

Female 8-10-week-old BALB/c mice were purchased from Charles River Laboratories (Kent, United Kingdom). Mice were immunised a total of four times with 30 days intervals. A total volume of 50 μl of PBS containing 50 μg of plasmid DNA was administered via subcutaneous route in the rear flank. Blood was sampled from the saphenous vein at 15 days intervals, and animals were terminally bled by cardiac puncture under non-recovery anaesthesia at day 150.

Vaccine Boost Efficacy Studies in K18 hACE2 Mice.
Intradermal Nucleic Acid Immunisation with Tropis PharmaJet Delivery in Guinea Pigs.

Female 7-week-old Dunkin Hartley Guinea pigs (Envigo RMS, Blackthorn, United Kingdom) were immunised a total of three times with 30 days intervals. A total volume of 200 μl of PBS containing 400 μg of plasmid DNA was administered by PharmaJet Tropis intradermal device. 100 μl was administered to each hind leg. Blood was sampled from the saphenous vein at 15 days intervals.

Intradermal Nucleic Acid Immunisation with Tropis PharmaJet Delivery in in Rabbits.

Ten mature (five male, five female) rabbits were immunised with a GMP lot pEVAC_T2_17 (clinical pEVAC_PS) i.d. by PharmaJet Tropis needleless delivery to the upper left and right hind limbs (300 μl at 2 mg/mL). Arterial blood was sampled at 14 days intervals

ACE-2 competition assay

The SARS-CoV-2 surrogate virus neutralisation test (SVNT, Genscript, Location, country) was carried out as per manufacturer's instructions. Briefly, serum from bleed 6 guinea pigs were diluted in PBS across an 8 point 1:2 dilution series from a starting concentration of 1:50. Samples were further diluted in the provided sample buffer at a 1:9 ratio, and then mixed with HRP conjugated to SARS-CoV-2 RBD protein, incubated at 37° C. for 30 min and added to human ACE-2 protein coated wells in 96-well plate format. The reaction was incubated at 37° C. for 15 min and then washed four times with provided wash buffer. TMB solution was then added, incubated for 15 minutes in the dark at R.T to allow the reaction to develop. The reaction was then quenched using the provided stop solution, and then absorbance read at 450 nm.

### Statistical Analyses

Mann-Whitney U test was performed for all the comparison using python sklearn package¹³. All the plots were generated using python Matplotlib package¹⁴.

### REFERENCES

1. Edgar, R. C. MUSCLE: a multiple sequence alignment method with reduced time and space complexity. BMC Bioinformatics 5, 113 (2004).
2. Nguyen, L.-T., Schmidt, H. A., von Haeseler, A. & Minh, B. Q. IQ-TREE: A Fast and Effective Stochastic Algorithm for Estimating Maximum-Likelihood Phylogenies. Mol. Biol. Evol. 32, 268-274 (2015).
3. Pond, S. L. K., Frost, S. D. W. & Muse, S. V. HyPhy: hypothesis testing using phylogenies. Bioinformatics 21, 676-679 (2005).
4. Berman, H. M. et al. The Protein Data Bank. Nucleic Acids Res. 28, 235-242 (2000).
5. Eswar, N. et al. Comparative protein structure modeling using MODELLER. Curr. Protoc. Protein Sci. Chapter 2, Unit 2.9 (2007).
6. Sali, A. & Blundell, T. L. Comparative protein modelling by satisfaction of spatial restraints. J. Mol. Biol. 234, 779-815 (1993).
7. Shen, M. & Sali, A. Statistical potential for assessment and prediction of protein structures. Protein Sci. Publ. Protein Soc. 15, 2507-2524 (2006).
8. Krivov, G. G., Shapovalov, M. V. & Dunbrack, R. L. Improved prediction of protein side-chain conformations with SCWRL4: Side-Chain Prediction with SCWRL4. Proteins Struct. Funct. Bioinforma. 77, 778-795 (2009).
9. Van Der Spoel, D. et al. GROMACS: fast, flexible, and free. J. Comput. Chem. 26, 1701-1718 (2005).
10. Schymkowitz, J. et al. The FoldX web server: an online force field. Nucleic Acids Res. 33, W382-W388 (2005).
11. Amanat, F. et al. A serological assay to detect SARS-CoV-2 seroconversion in humans. Nat. Med. 26, 1033-1036 (2020).
12. Carnell, G., Grehan, K., Ferrara, F., Molesti, E. & Temperton, N. J. An Optimised Method for the Production using PEI, Titration and Neutralization of SARS-CoV Spike Luciferase Pseudotypes. Bio-Protoc. 7, (2017).
13. Pedregosa, F. et al. Scikit-learn: Machine Learning in Python. J. Mach. Learn. Res. 12, 2825-2830 (2011).
14. Hunter, J. D. Matplotlib: A 2D Graphics Environment. Comput. Sci. Eng. 9, 90-95 (2007).

### Supplementary Information

### FIG. 46—Multiple Sequence Alignment of the Known Sarbecoviruses

Sarbecoviruses are divided into two distinct phylogenetic clades—clade 1 (boxed in blue) and clade 2. Members of clade 1 has deletions around the ACE-2 binding motif and have been reported to not bind human ACE-2 receptor. The regions corresponding to epitope region of S309, CR3022 and B38 antibodies are coloured in grey, purple, and orange respectively.

FIG. 47A—Elisa Binding Data of K18 hACE2 Sera

Binding antibodies were observed 4 weeks post immunisation with AZD1222 and 4 weeks post boosting with different AZD1222/T2_17/SARS2_RBD.

FIG. 48B—Neutralisation Data of K18 hACE2 Sera

Neutralising antibodies against SARS-CoV-1 and delta variant of SARS-CoV-1 were observed two-week post boost (bleed 3) and the levels were maintained 6 weeks post boost (bleed 5).

FIG. 49—Neutralisation data for SARS2_RBD_P521N and SARS2_RBD in BALB/c Mice

Sera from BALB/c mice immunised with SARS2_RBD_P521N and SARS-2 RBD generated similar neutralising antibody response 15 days post four immunisations. The difference is statistically non-significant (Mann-Whitney U test, p-value=0.4681).

### EXAMPLE 36

### T2_17+pEVAC Expression Vector (SEQ ID NO:86)

This example provides the nucleic acid sequence encoding the T2_17 vaccine construct (amino acid sequence SEQ ID NO:31; nucleic acid sequence SEQ ID NO:82) within the pEVAC expression vector.

CCTCTGACACATGCAGCTCCCGGAGACGGTCACAGCTTGTCTGTAAGCGGATGCCGGGAGCAGACAAG
CCCGTCAGGGCGCGTCAGCGGGTGTTGGCGGGTGTCGGGGCTGGCTTAACTATGCGGCATCAGAGCAG
ATTGTACTGAGAGTGCACCATATGCGGTGTGAAATACCGCACAGATGCGTAAGGAGAAAATACCGCAT
CAGATTGGCTATTGGCCATTGCATACGTTGTATCCATATCATAATATGTACATTTATATTGGCTCATG
TCCAACATTACCGCCATGTTGACATTGATTATTGACTAGTTATTAATAGTAATCAATTACGGGGTCAT
TAGTTCATAGCCCATATATGGAGTTCCGCGTTACATAACTTACGGTAAATGGCCCGCCTGGCTGACCG
CCCAACGACCCCCGCCCATTGACGTCAATAATGACGTATGTTCCCATAGTAACGCCAATAGGGACTTT
CCATTGACGTCAATGGGTGGAGTATTTACGGTAAACTGCCCACTTGGCAGTACATCAAGTGTATCATA
TGCCAAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGTGATGCG
GTTTTGGCAGTACATCAATGGGCGTGGATAGCGGTTTGACTCACGGGGATTTCCAAGTCTCCACCCCA
TTGACGTCAATGGGAGTTTGTTTTGGCACCAAAATCAACGGGACTTTCCAAAATGTCGTAACAACTCC
GCCCCATTGACGCAAATGGGCGGTAGGCGTGTACGGTGGGAGGTCTATATAAGCAGAGCTCGTTTAGT
GAACCGTCAGATCGCCTGGAGACGCCATCCACGCTGTTTTGACCTCCATAGAAGACACCGGGACCGAT
CCAGCCTCCATCGGCTCGCATCTCTCCTTCACGCGCCCGCCGCCCTACCTGAGGCCGCCATCCACGCC
GGTTGAGTCGCGTTCTGCCGCCTCCCGCCTGTGGTGCCTCCTGAACTGCGTCCGCCGTCTAGGTAAGT
TTAAAGCTCAGGTCGAGACCGGGCCTTTGTCCGGCGCTCCCTTGGAGCCTACCTAGACTCAGCCGGCT
CTCCACGCTTTGCCTGACCCTGCTTGCTCAACTCTAGTTAACGGTGGAGGGCAGTGTAGTCTGAGCAG
TACTCGTTGCTGCCGCGCGCGCCACCAGACATAATAGCTGACAGACTAACAGACTGTTCCTTTCCATG
GGTCTTTTCTGCAGTCACCGTCGGTACCGCCACCATGGATGCTATGAAGAGGGGCCTGTGCTGCGTGC
TGCTTCTGTGTGGCGCTGTGTTTGTGTCTCCTAGCGCCGCTAGAGTGGCCCCTACCAAAGAAGTCGTG
CGGTTCCCCAACATCACCAATCTGTGCCCTTTCGGCGAGGTGTTCAACGCCACCAAGTTTCCCTCTGT
GTACGCCTGGGAGCGCAAAAAGATCAGCAACTGCGTGGCCGACTACAGCGTGCTGTACAACAGCACCA
GCTTCAGCACCTTCAAGTGCTACGGCGTGTCACCCACCAAGCTGAACGACCTGTGCTTCACCAACGTG
TACGCCGACAGCTTCGTGATCAGAGGCGACGAAGTGCGGCAGATTGCCCCTGGACAAACAGGCGTGAT
CGCCGATTACAACTACAAGCTGCCCGACGACTTCACCGGCTGTGTGATCGCCTGGAACACCAACAACA
TCGACAGCACCACCGGCGGCAACTACAACTACCTGTACAGAAGCCTGCGGAAGTCTAAGCTGAAGCCC
TTCGAGCGGGACATCAGCAGCGACATCTATAGCCCTGGCGGCAAGCCTTGTTCTGGCGTGGAAGGCTT
CAACTGCTACTACCCTCTGCGGAGCTACGGCTTCTTCCCCACAAATGGCACAGGCTACCAGCCTTACA
GAGTGGTGGTCCTGAGCTTCGAGCTGCTGAATGCCCCTGCCACAGTGTGTGGCCCTAAGCTGTCTACC
GACTGAGCGGCCGCAGATCTGCTGTGCCTTCTAGTTGCCAGCCATCTGTTGTTTGCCCCTCCCCCGTG
CCTTCCTTGACCCTGGAAGGTGCCACTCCCACTGTCCTTTCCTAATAAAATGAGGAAATTGCATCGCA
TTGTCTGAGTAGGTGTCATTCTATTCTGGGGGGTGGGGTGGGGCAGGACAGCAAGGGGGAGGATTGGG
AAGACAATAGCAGGCATGCTGGGGATGCGGTGGGCTCTATGGCTACCCAGGTGCTGAAGAATTGACCC
GGTTCCTCCTGGGCCAGAAAGAAGCAGGCACATCCCCTTCTCTGTGACACACCCTGTCCACGCCCCTG
GTTCTTAGTTCCAGCCCCACTCATAGGACACTCATAGCTCAGGAGGGCTCCGCCTTCAATCCCACCCG
CTAAAGTACTTGGAGCGGTCTCTCCCTCCCTCATCAGCCCACCAAACCAAACCTAGCCTCCAAGAGTG
GGAAGAAATTAAAGCAAGATAGGCTATTAAGTGCAGAGGGAGAGAAAATGCCTCCAACATGTGAGGAA
GTAATGAGAGAAATCATAGAATTTTAAGGCCATGATTTAAGGCCATCATGGCCTTAATCTTCCGCTTC
CTCGCTCACTGACTCGCTGCGCTCGGTCGTTCGGCTGCGGCGAGCGGTATCAGCTCACTCAAAGGCGG
TAATACGGTTATCCACAGAATCAGGGGATAACGCAGGAAAGAACATGTGAGCAAAAGGCCAGCAAAAG
GCCAGGAACCGTAAAAAGGCCGCGTTGCTGGCGTTTTTCCATAGGCTCCGCCCCCCTGACGAGCATCA
CAAAAATCGACGCTCAAGTCAGAGGTGGCGAAACCCGACAGGACTATAAAGATACCAGGCGTTTCCCC
CTGGAAGCTCCCTCGTGCGCTCTCCTGTTCCGACCCTGCCGCTTACCGGATACCTGTCCGCCTTTCTC
CCTTCGGGAAGCGTGGCGCTTTCTCATAGCTCACGCTGTAGGTATCTCAGTTCGGTGTAGGTCGTTCG
CTCCAAGCTGGGCTGTGTGCACGAACCCCCCGTTCAGCCCGACCGCTGCGCCTTATCCGGTAACTATC
GTCTTGAGTCCAACCCGGTAAGACACGACTTATCGCCACTGGCAGCAGCCACTGGTAACAGGATTAGC
AGAGCGAGGTATGTAGGCGGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAAGAGTTGGTAGCTCTTGAT
CCGGCAAACAAACCACCGCTGGTAGCGGTGGTTTTTTTGTTTGCAAGCAGCAGATTACGCGCAGAAAA
AAAGGATCTCAAGAAGATCCTTTGATCTTTTCTACGGGGTCTGACGCTCAGTGGAACGAAAACTCACG
TTAAGGGATTTTGGTCATGAGATTATCAAAAAGGATCTTCACCTAGATCCTTTTAAATTAAAAATGAA
GTTTTAAATCAATCTAAAGTATATATGAGTAAACTTGGTCTGACAGTTACCAATGCTTAATCAGTGAG
GCACCTATCTCAGCGATCTGTCTATTTCGTTCATCCATAGTTGCCTGACTCGGGGGGGGGGGGCGCTG
AGGTCTGCCTCGTGAAGAAGGTGTTGCTGACTCATACCAGGCCTGAATCGCCCCATCATCCAGCCAGA
AAGTGAGGGAGCCACGGTTGATGAGAGCTTTGTTGTAGGTGGACCAGTTGGTGATTTTGAACTTTTGC
TTTGCCACGGAACGGTCTGCGTTGTCGGGAAGATGCGTGATCTGATCCTTCAACTCAGCAAAAGTTCG
ATTTATTCAACAAAGCCGCCGTCCCGTCAAGTCAGCGTAATGCTCTGCCAGTGTTACAACCAATTAAC
CAATTCTGATTAGAAAAACTCATCGAGCATCAAATGAAACTGCAATTTATTCATATCAGGATTATCAA
TACCATATTTTTGAAAAAGCCGTTTCTGTAATGAAGGAGAAAACTCACCGAGGCAGTTCCATAGGATG
GCAAGATCCTGGTATCGGTCTGCGATTCCGACTCGTCCAACATCAATACAACCTATTAATTTCCCCTC
GTCAAAAATAAGGTTATCAAGTGAGAAATCACCATGAGTGACGACTGAATCCGGTGAGAATGGCAAAA
GCTTATGCATTTCTTTCCAGACTTGTTCAACAGGCCAGCCATTACGCTCGTCATCAAAATCACTCGCA
TCAACCAAACCGTTATTCATTCGTGATTGCGCCTGAGCGAGACGAAATACGCGATCGCTGTTAAAAGG
ACAATTACAAACAGGAATCGAATGCAACCGGCGCAGGAACACTGCCAGCGCATCAACAATATTTTCAC
CTGAATCAGGATATTCTTCTAATACCTGGAATGCTGTTTTCCCGGGGATCGCAGTGGTGAGTAACCAT
GCATCATCAGGAGTACGGATAAAATGCTTGATGGTCGGAAGAGGCATAAATTCCGTCAGCCAGTTTAG
TCTGACCATCTCATCTGTAACATCATTGGCAACGCTACCTTTGCCATGTTTCAGAAACAACTCTGGCG
CATCGGGCTTCCCATACAATCGATAGATTGTCGCACCTGATTGCCCGACATTATCGCGAGCCCATTTA
TACCCATATAAATCAGCATCCATGTTGGAATTTAATCGCGGCCTCGAGCAAGACGTTTCCCGTTGAAT
ATGGCTCATAACACCCCTTGTATTACTGTTTATGTAAGCAGACAGTTTTATTGTTCATGATGATATAT
TTTTATCTTGTGCAATGTAACATCAGAGATTTTGAGACACAACGTGGCTTTCCCCCCCCCCCCATTAT
TGAAGCATTTATCAGGGTTATTGTCTCATGAGCGGATACATATTTGAATGTATTTAGAAAAATAAACA
AATAGGGGTTCCGCGCACATTTCCCCGAAAAGTGCCACCTGACGTCTAAGAAACCATTATTATCATGA
CATTAACCTATAAAAATAGGCGTATCACGAGGCCCTTTCGTCTCGCGCGTTTCGGTGATGACGGTGAA
AA

### EXAMPLE 37

### Digitally Immune Optimised Spike Vaccine Induces Broad Neutralising Responses Against SARS-CoV-2 Variants of Concern

### SUMMARY

Successive waves of SARS-CoV-2 variants of concern (VOC) have increased ability to escape existing immunity in vaccinated and infected populations. Current licensed SARS-CoV-2 vaccines have a reduced ability to elicit or boost neutralising antibodies against the most recent variants. New vaccine strategies are needed that can induce broad protective immunity across the VOCs. The evolution of SARS-CoV-2 variants can be re-capitulated from the detailed global surveillance efforts, and epidemiological sequence data on the VOCs. Using this data, we undertook a structure-based approach to computationally generate artificial Spike genes designed to induce neutralising antibody responses across a spectrum of VOCs. The study was a VOC mutation informed spike termed T2_29, with multiple versions, such as a Q498R mutant, a mutation later to be acquired by the Omicron lineage VOCs and a C-terminal truncated and Q498R mutant. Three DNA immunisations in Guinea pigs between the C-terminal truncated ancestral spike and T2_29 Spike with or without the C-terminal truncation and Q498R mutation, revealed superior immune response across the VOCs by T2_29 and modified T2_29 constructs in comparison to the C-terminal truncated ancestral construct. We further boosted all the groups with MVA expressing T2_29 with C-terminal truncated and Q498R modifications. MVA boost significantly increased the immune response across the groups against all the variants tested for.

### INTRODUCTION

Over the past two years SARS-CoV-2 has acquired many spike mutations with different degree of effects on its interaction with the host, and its ability to escape pre-existing human immune responses acquired by vaccination and or infection. In addition to the evolution of SARS-CoV-2 in humans, it has been reported that the virus has transmitted to other mammals such as mink, cat, dogs, and certain species of deer. Cross-species infections of SARS-CoV-2, in which species-specific variants occur, provide an additional dimension to rate of evolution, their fitness, and immune escape features that may enable future SARS-CoV-2 variant epidemics. Since late 2020, many variants of concern (VOCs) have been reported, starting with the Alpha, Beta, Gamma, Delta, and the most recent variants of the Omicron lineage. It is the evolution of the spike protein, that enables immune escape and evasion that are influenced by several different selective pressures including immune pressure. The emergence of adaptive mutations present in the S protein can strongly affect host tropism and viral transmission. Facing an increasingly immune population, immunological escape from host immunity acquired during prior infection and/or vaccination is required for future variants to acquire advantageous changes allowing them to replicate and spread in the immune human population. With the emergence of each subsequent variant of concern (VOC), there has been a declining level of vaccine induced neutralising antibodies induced by the ancestral Spike antigen (Wuhan Hu-1 strain) used by all the current generation of COVID-19 vaccines. Out of these, Delta, and Omicron, and Omicron sub-variants have been reported with higher transmission rates and immune escape from both naturally as well as vaccine acquired immunity. This necessitates an urgent update of current SARS-CoV-2 vaccines, which still use the ancestral strain. Continued use of vaccines based on ancestral sequence have a diminishing effect on facilitating de novo responses to new epitopes of new variants. Leading COVID-19 mRNA vaccine manufacturers have added an Omicron BA.1 Spike antigen to their Wuhan Spike based vaccine as a bivalent vaccine to provide better protection against the Omicron lineage of variants by adapting their vaccine to omicron lineage and administered either as a monovalent vaccine or a bivalent vaccine. Adapting the vaccine to a specific lineage can be beneficial to provide protection against a new emerging variant from the vaccine matched lineage but it may not provide desirable protection against emerging antigenically different lineages of SARS-CoV-2 or re-emergence of already reported antigenically distinct lineages of SARS-CoV-2. To circumvent this problem, we have developed a new single Spike-based vaccine antigen that expresses diverse epitopes covering majority of the VOCs known at the time of its devising (comprising Alpha, Beta, and Gamma lineages). This novel vaccine antigen, T2_29 (SEQ ID NO:53), demonstrated considerable neutralising breadth against SARS-CoV-2 pseudotypes expressing the ancestral Wuhan spike, as well as pseudoviruses expressing Alpha, Beta, Gamma, and Delta lineage S proteins, and pseudoviruses of Omicron BA.1, BA.2, and BA.4/5 variants.

As explained in more detail below, we have also designed full length S protein COV_S_T2_29 with arginine residue at position 498 (i.e. Q498R) (SEQ ID NO:87), which corresponds to position 501, of SEQ ID NO:52. The amino acid sequence of the designed full-length S protein sequence is given below.

>COV_S_T2_29 + Q498R
(SEQ ID NO: 87)
MFVFLVLLPLVSSQCVNFTNRTQLPSAYTNSFTRGVYYPDKVFRSSVLHSTQDLFLPFFSNVTWFHAI--SG
TNGTKRFDNPVLPFNDGVYFASTEKSNIIRGWIFGTTLDSKTQSLLIVNNATNVVIKVCEFQFCNDPFLGV-
YHKNNKSWMESEFRVYSSANNCTFEYVSQPFLMDLEGKQGNFKNLREFVFKNIDGYFKIYSKHTPINLVR--
-DLPQGFSALEPLVDLPIGINITRFQTLLALHRSYLTPGDSSSGWTAGAAAYYVGYLQPRTFLLKYNENGTI
TDAVDCALDPLSETKCTLKSFTVEKGIYQTSNFRVQPTESIVRFPNITNLCPFGEVFNATRFASVYAWNRKR
ISNCVADYSVLYNSASFSTFKCYGVSPTKLNDLCFTNVYADSFVIRGDEVRQIAPGQTGNIADYNYKLPDDF
TGCVIAWNSNNLDSKVGGNYNYLYRLFRKSNLKPFERDISTEIYQAGSTPCNGVKGFNCYFPLQSYGFRPTY
GVGYQPYRVVVLSFELLHAPATVCGPKKSTNLVKNKCVNFNFNGLTGTGVLTESNKKFLPFQQFGRDIADTT
DAVRDPQTLEILDITPCSFGGVSVITPGTNTSNQVAVLYQGVNCTEVPVAIHADQLTPTWRVYSTGSNVFQT
RAGCLIGAEHVNNSYECDIPIGAGICASYQTQTNSHRRARSVASQSIIAYTMSLGAENSVAYSNNSIAIPTN
FTISVTTEILPVSMTKTSVDCTMYICGDSTECSNLLLQYGSFCTQLNRALTGIAVEQDKNTQEVFAQVKQIY
KTPPIKDFGGFNFSQILPDPSKPSKRSFIEDLLFNKVTLADAGFIKQYGDCLGDIAARDLICAQKFNGLTVL
PPLLTDEMIAQYTSALLAGTITSGWTFGAGAALQIPFAMQMAYRFNGIGVTQNVLYENQKLIANQFNSAIGK
IQDSLSSTASALGKLQDVVNQNAQALNTLVKQLSSNFGAISSVLNDILSRLDPPEAEVQIDRLITGRLQSLQ
TYVTQQLIRAAEIRASANLAATKMSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNCDVVIGIVNNTVYDPLQPELDSFK
EELDKYFKNHTSPDVDLGDISGINASVVNIQKEIDRLNEVAKNLNESLIDLQELGKYEQYIKWPWYIWLGFI
AGLIAIVMVTIMLCCMTSCCSCLKGCCSCGSCCKFDEDDSEPVLKGVKLHYT

We have also designed full length S protein COV_S_T2_29+Q498R+dER (SEQ ID NO:88), wherein COV_S_T2_29+Q498R also has a C-terminal truncation (deletion of the ER signal sequence). The amino acid sequence of the designed full-length S protein sequence is given below.

>COV_S_T2_29 + Q498R + dER
(SEQ ID NO: 88)
MFVFLVLLPLVSSQCVNFTNRTQLPSAYTNSFTRGVYYPDKVFRSSVLHSTQDLFLPFFSNVTWFHAI--SG
TNGTKRFDNPVLPFNDGVYFASTEKSNIIRGWIFGTTLDSKTQSLLIVNNATNVVIKVCEFQFCNDPFLGV-
YHKNNKSWMESEFRVYSSANNCTFEYVSQPFLMDLEGKQGNFKNLREFVFKNIDGYFKIYSKHTPINLVR--
-DLPQGFSALEPLVDLPIGINITRFQTLLALHRSYLTPGDSSSGWTAGAAAYYVGYLQPRTFLLKYNENGTI
TDAVDCALDPLSETKCTLKSFTVEKGIYQTSNFRVQPTESIVRFPNITNLCPFGEVFNATRFASVYAWNRKR
ISNCVADYSVLYNSASFSTFKCYGVSPTKLNDLCFTNVYADSFVIRGDEVRQIAPGQTGNIADYNYKLPDDF
TGCVIAWNSNNLDSKVGGNYNYLYRLFRKSNLKPFERDISTEIYQAGSTPCNGVKGFNCYFPLQSYGFRPTY
GVGYQPYRVVVLSFELLHAPATVCGPKKSTNLVKNKCVNFNFNGLTGTGVLTESNKKFLPFQQFGRDIADTT
DAVRDPQTLEILDITPCSFGGVSVITPGTNTSNQVAVLYQGVNCTEVPVAIHADQLTPTWRVYSTGSNVFQT
RAGCLIGAEHVNNSYECDIPIGAGICASYQTQTNSHRRARSVASQSIIAYTMSLGAENSVAYSNNSIAIPTN
FTISVTTEILPVSMTKTSVDCTMYICGDSTECSNLLLQYGSFCTQLNRALTGIAVEQDKNTQEVFAQVKQIY
KTPPIKDFGGFNFSQILPDPSKPSKRSFIEDLLFNKVTLADAGFIKQYGDCLGDIAARDLICAQKFNGLTVL
PPLLTDEMIAQYTSALLAGTITSGWTFGAGAALQIPFAMQMAYRFNGIGVTQNVLYENQKLIANQFNSAIGK
IQDSLSSTASALGKLQDVVNQNAQALNTLVKQLSSNFGAISSVLNDILSRLDPPEAEVQIDRLITGRLQSLQ
TYVTQQLIRAAEIRASANLAATKMSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNCDVVIGIVNNTVYDPLQPELDSFK
EELDKYFKNHTSPDVDLGDISGINASVVNIQKEIDRLNEVAKNLNESLIDLQELGKYEQYIKWPWYIWLGFI
AGLIAIVMVTIMLCCMTSCCSCLKGCCSCGSCC-------------------

### Methods

### In-Silico Design of the Vaccine Antigen

A consensus sequence was generated for each VOCs viz. Alpha, Beta, and Gamma using the sequences deposited in NCBI virus. Each mutation was mapped to the different regions of Spike and clusters of mutation from different structural domains were sequentially combined to generate the next generation spike-based vaccine antigen. The structural integrity of the resultant vaccine antigen was checked for by generating homology models using Modeller software.

### Production and Transformation of Plasmids

Sequences of vaccine designs were gene-optimized and adapted to human codon use via the GeneOptimizer algorithm. These genes were cloned into pEVAC (GeneArt, Germany) via restriction digestion. Plasmids were transformed via heat-shock in chemically induced competent E. coli DH5a cells (Invitrogen 18265-017). Plasmid DNA was extracted from transformed bacterial cultures via the Plasmid Mini Kit (Qiagen 12125). All plasmids were subsequently quantified using UV spectrophotometry (NanoDrop™-Thermo Scientific).

### Vaccination Experiments in Guinea Pigs

Four groups of four seven-week-old female Hartley guinea pigs were purchased from Envigo (Maastricht, Netherlands). Guinea pigs were immunised at two-week intervals with 200 μg DNA vaccines bearing the antigen gene in the pURVac vector, administered by intradermal route using the Pharmajet® device in a total volume of 200 μl over the hind legs. Animals were given three doses of DNA by the same route and then boosted with MVA by intramuscular route at a dose of 1e7 PFU/dose seven weeks after the first three doses. Bleeds were taken through the saphenous vein at two weeks intervals.

### Production of Lentiviral Pseudotypes

Lentiviral pseudotypes were produced by transient transfection of HEK293T/17 cells with packaging plasmids p8.91 and pCSFLW and different spike expression plasmids bearing the using the Fugene-HD transfection reagent. Supernatants were taken after 48h, filtered at 0.45 μm and titrated on HEK293T/17 cells transiently expressing human ACE-2 and TMPRSS2. Target cells used were HEK293T/17 cells transfected 24h prior with 2 μg huACE-2 and 75 ng TMPRSS2.

### Pseudotype-Based Micro-Neutralisation Assay

Pseudotype-based micro-neutralisation assay was performed as described previously. Briefly, serial dilutions of serum were incubated with SARS-CoV-2 spike bearing lentiviral pseudotype for 1 h at 37° C., 5% CO2 in 96-well white cell culture plates. 1.5×104 HEK293T/17 transiently expressing human ACE-2 and TMPRSS2 were then added per well and plates incubated for 48 hrs at 37° C., 5% CO2 in a humidified incubator. Bright-Glo (Promega) was then added to each well and luminescence read after a five-minute incubation period. Experimental data points were normalised to 100% and 0% neutralisation controls and non-linear regression analysis performed in GraphPad Prism 9 to produce neutralisation curves and IC50 values.

### Statistical Analyses

Two-tailed Mann-Whitney U tests were performed for all the comparisons using the Python sklearn package. All the plots were generated using the Python Matplotlib package.

### Results

### In-Silico Design of the Vaccine Antigen

The structure of the spike protein of SARS-CoV-2 can be antigenically divided into three distinct regions,—the N-terminal domain (NTD), the receptor binding domain (RBD), and the stalk region. The RBD possesses most of the experimentally characterised epitopes, followed by the NTD and the stalk. The relevance of these epitopes in protection from SARS-CoV-2 can also be appreciated from the observation of multiple mutations in the RBD and NTD in the SARS-CoV-2 VOCs. For our variant vaccine antigen, we clustered all the reported mutations in the VOCs. Alpha, Beta, and Gamma into NTD, RBD, and stalk regions. It is important to note that for our bioinformatics analyses, we consider the epitopes in NTD, RBD, and stalk to be non-synergistic in eliciting immune responses and immune responses to these domains would be independent of each other. Once, epitopes were clustered, antigens were generated by sequential combination of different VOC specific mutations in NTD, RBD, and stalk. Only mutations that were reported in immunodominant regions were considered for the design. Based on these combinations, the Spike vaccine antigen—T2_29 (FIG. 49) was generated using available data on Alpha, Beta, and Gamma variants. The T2_29 modified Spike was further modified to three other antigens viz. T2_29+Q, and T2_29+Q+dER. The mutation Q498R was observed to be prominent in the circulating SARS-CoV-2 variants of interest prior to April 2021 and was included on the backbone of T2_29 to give T2_29+Q design as a pre-emptive antigen design for future variants. It is interesting to note that the Q498R mutation was later acquired by the Omicron variants in late 2021. A C-terminal deletion version of the T2_29+Q was also generated for comparison. Deletion of 19 amino acid from C-terminal was reported to express the spike protein on the surface of cell better in comparison to full-length and hence higher antigen presentation. We also deleted this C-terminal region from the WT ancestral antigen as a control, henceforth referred as WTdER. All these vaccine antigens have the stabilising double Proline mutations, as reported in majority of the current vaccines.

### Spike Vaccine Antigen Delivered by DNA and MVA in Guinea Pigs

Guinea pigs were immunised thrice with the antigens—T2_29, T2_29+Q, T2_29+Q+dER, and WTdER in DNA vector and were boosted with MVA expressing T2_29+Q+dER once (FIG. 50A). The neutralising titres were longitudinally analysed for WTdER against pseudoviruses (PVs) expressing VOC spikes. The neutralising antibodies peaked at bleed 4 following three immunisation and bleed 6 following MVA boost (FIG. 50B). The neutralising titre against all the VOCs and the ancestral sequence were measured for these bleeds (FIGS. 50C and 50D). The first-generation spike vaccine antigen—T2_29 and its modifications viz. T2_29+Q, T2_29+Q+dER were able to induce broad neutralising response against all the VOCs tested. The T2_29 based antigens generated at least two-fold better neutralising response against Alpha, Beta, Gamma, and Omicron in comparison to WTdER (FIG. 50C) after three doses of DNA vaccine. The neutralising antibody titres against both the Ancestral sequence and Delta were comparable to WTdER (FIG. 50C) for T2_29 and T2_29+Q+dER. But a lower titre was observed for T2_29+Q before MVA boost. The WTdER generated a very weak neutralising antibody titre against the Omicron but all our vaccine antigens generated a robust neutralising antibody response against Omicron. Interestingly, the T2_29+Q showed lower neutralising titre to Omicron in comparison to T2_29 and T2_29+Q_dER. We believe the higher neutralising titre of the T2_29+Q_dER in comparison to T2_29+Q is due to higher expression of the construct. It is important to note that T2_29 doesn't include many of the mutations reported in Delta and Omicron variants, as these were designed prior to the outbreak of Delta and Omicron. Despite of lacking many of the important mutations reported in Delta and Omicron neutralising, T2_29 induced high titres against Omicron and titres comparable to wild type for Delta. We further boosted all the groups of guinea pigs with MVA expressing T2_29+Q+dER. We chose this specific construct, as this was antigenically closer to the omicron BA.1. On boosting with MVA, the neutralisation titre of all the vaccine antigens significantly increased (FIG. 50D). Most importantly, the neutralising titre of the WTdER against Omicron BA.1 increased by 3-fold on boosting with MVA expressing T2_29+Q+dER. This strongly supports the applicability of these spike antigens for boosting the immune response against emerging variants in the already vaccinated or infected human population.

### Discussion

Advancement in the field of vaccine technology and genomics had successfully led to the development and distribution of vaccines against COVID-19. These vaccines have been successful in controlling the spread of COVID-19 as well as the mortality rate due to COVID-19, but the rapid emergence of new variants of SARS-CoV-2 has caused worrying trends in the infection rate and associated hospitalization. The variants are observed to escape from immune response generated either by natural infection or vaccination, causing infections as well as re-infection. This has led to further boosting of the immune response by immunizing the population with booster doses of the original vaccine. Although the booster results in increased antibody titre, it still may be ineffective against emerging variants. In view of this many vaccine manufacturing companies have rolled out vaccines with updated antigens to include the recent variants, or a combination of the original Wuhan-Hu-1 based antigen and the recent Omicron BA.1 variant. These may provide protection against circulating variants but may fail against new variants that are phylogenetically different from the current circulating variants, or against new variants that are phylogenetically alike to already known variants. Here we describe novel spike-based antigens that incorporate the information on the mutations observed across the reported VOCs known at the time of devising. We designed our spike antigen using mutations observed in Alpha, Beta, and Gamma and validated the immunogenicity of the design in Guinea pigs using DNA/MVA vaccination regime. Robust neutralising titre were observed after three DNA vaccinations. T2_29 generated a superior neutralising response to all the tested VOCs except Delta, where it was comparable to the Ancestral Wuhan-Hu-1 antigen. Interestingly and importantly, elicitation of comparable and superior immune response to Delta and Omicron BA. 1 by T2_29 is encouraging and validate our rationale that the novel spike antigens that include mutation information across the VOCs would be better vaccine antigen against emerging variants in comparison to natural variant sequence. From the data presented we can conclude that antigenically engineered Spike genes may induce superior immune breadth than combinations of original and variant Spike antigens.

### Supplementary Information

Background: Current COVID-19 Vaccines are based on wild-type Spike SARS-CoV-2, from the original Wuhan sequence or the Omicron BA. 1 Spike variant.

Problem: As new variants continue to emerge, the use of “historical” Spike antigens from past waves of SARS-CoV-2 variants will have reduced benefit on prevention of the emergence of new variants. Thus repeated boosting of immune responses from past immunisations or infections (“Original Antigenic Sin”) may have a diminishing effect on facilitating de novo responses to new epitopes to prevent the emergence of new variants.

Objective: To determine if a single engineered SARS-CoV-2 Spike design expressing diverse epitopes is capable of inducing neutralization across the SARS-CoV-2 Variants of Concern (VOCs), and to increase the breadth of protective immunity that can be achieved by novel immunogens to protect against future SARS-CoV-2 variants.

### Study 1a:

Neutralising antibody titres in outbred Guinea pigs after DNA immunisation with SARS-CoV-2 Spikes designed with VOC mutations.

### Study Design:

Group 1. a full spike construct based on the original Wuhan sequence of SARS-CoV-2 with a deletion of the ER retention signal

Group 2a: T2_29, Group 2b: T2_29+Q, Group 2c: T2_29+Q+dER mutation (all in combination with N501Y)

### Results:

FIG. 51 shows VOC RBD binding antibody levels of guinea pigs at bleed 4, shown by ELISA. The area under the curve (=AUC) calculated from the logarithmic dilution curves is plotted against the different vaccine constructs. A-F: Binding to each of the RBD variants is plotted. Overall signal strength varies between RBD variants and thus a comparison can only be made within one variant RBD. For each group the 4 individual values and the mean with 95% CI were plotted.

As shown, at bleed 4, sera of Guinea pigs immunised with the WT spike DNA construct demonstrated the second highest level of neutralisation (mean IC₅₀=1,438) against the PV carrying the homologous WT spike. Neutralisation against the alpha PV was higher than against the wildtype (mean IC₅₀=3,844).

When assayed against the VOCs delta (573), beta (94), gamma (46), and omicron BA. 1/BA.2 (15, 26), WTΔER shows a continuous decrease in mean nAb titres with almost no neutralisation against omicron. Compared with all three group 2 Super spikes (2a, 2b, 2c) with the group 1 WTΔER immunised group, there is a significant increase in mean IC₅₀ nAb values when assayed against beta (**, p<0.01), gamma (**, p<0.005), omicron BA.1 (*, p<0.05), and omicron BA.2 (*, p<0.05). T2_29+Q, one of the two constructs carrying the additional Q498R mutation, shows lower levels of neutralisation (mean IC₅₀=295) against omicron BA.1 than T2_29 (mean IC₅₀=3,045).

The RBD of the T2_29 construct is identical to that of beta and almost identical to gamma with K417N instead of gamma's K417T. T2_29 shares three AA mutations with omicron and T2_29+Q (+/−ΔER) additionally includes omicron's Q498R, making them the genetically closest constructs to omicron in this study. The delta variant, on the other hand, carries two RBD mutations not found in the other VOC's (except T478K in BA.2) nor in any of the Super-spike designs. The delta RBD is therefore the most antigenically distant from the Super-spike constructs, especially those including Q498R.

FIG. 52 shows the distribution of the neutralisation titre of guinea pig serum (at bleed 4) against Ancestral and VOCs, after DNA immunisation using WT vaccine (WTdER) and T2_29 vaccine groups (2a, 2b, 2c; data combined). The x-axis represents the pseudoviruses test for neutralisation, and the y-axis represents the IC₅₀ values. The WT vaccine appears on the left for each coronavirus pseudovirus, and the combined T2_29 vaccine appears on the right for each coronavirus pseudotype.

Discussion: The significant differences in mean IC₅₀ values between the combined T2_29 groups (2a, 2b, 2c) against the WTΔER group reveal a strong increase in neutralisation against beta, gamma, and omicron over the WTΔER immunised group. The IC₅₀ levels against Beta and Gamma are very similar.

Neither the effect of NTD mutations nor that of N417 versus T417 in the PVs could be observed. The T2_29 group 2's nAb activity against the WT and delta variant PV are still similar to those of the WTΔER group.

The addition of VOC mutations is not a “zero-sum game” where any gain in neutralisation against one variant leads to an equal loss against another (Thesis LO March 2022).

The T2_29 groups reveal a strong increase in neutralisation against beta, gamma, and omicron over the WTΔER immunised group. The T2_29 group's nAb levels against the WT and delta variant PVs are still similar to those of the WTΔER group.

### Study 1b:

Neutralising antibody titres in MVA boosted DNA immunised Guinea pigs after with MVA T2_29+Q+dER.

### Study Design:

Group 1. DNA delivered WT spike+dER, all boosted with MVA T2_29+Q+dER

Group 2: DNA delivered gp 2a, 2b, 2c, all boosted with MVA T2_29+Q+dER

FIG. 53G shows an overview of 3×DNA and MVA boost immunisation and bleed schedule for Groups 1 and 2. Guinea pigs were immunised with plasmid DNA (Guinea pig icons with PharmaJet device shown in green) on days 0, 14, and 70. The fourth immunisation with MVA (Guinea pig with syringe) followed on day 113. Bleeds (blood drop icons) were taken before the start of the immunisations, 2 and 4 weeks after each immunisation, and at the point of sacrifice (Terminal bleed).

### Results:

FIG. 53A-F shows neutralisation data at bleed 6 for guinea pigs immunised with WT or designed DNA constructs and then boosted with MVA T2_29+Q+dER. The Figure shows neutralisation data for each vaccine construct when challenged with a panel of VOCs. The x-axis represents the pseudoviruses test for neutralisation, and the y-axis represents the IC50 values.

Group 1: Despite the heterologous boost with T2_29+Q+ΔER, the nAb levels of the WTΔER group at bleed 6 still strongly correlated with levels at bleed 4 (Spearman r=0.83, ****). No correlation (p>0.05) was found between the WTΔER group at bleed 6, T2_29+Q+ΔER group at bleed 4. Notably, the WT vaccinated group given a heterologous MVA T2_29+Q+ΔER boost was found to only partially expand variant neutralisation.

Group 2: As expected, the three group 2 (2a, 2b, 2c) MVA T2_29+Q+ΔER boosted groups show a very similar pattern of neutralisation as at bleed 4. T2_29 group neutralisation of BA. 1 PV was not boosted to the same degree as that of the beta and gamma PVs.

FIG. 50 shows a summary of the data for this example; spike vaccine antigen T2_29 delivered by DNA and MVA in Guinea pigs:

A. Bleed schedule of the Guinea pigs. B. Distribution of the neutralisation titre of the Guinea pigs against Ancestral virus pseudotype on immunisation with WTdER. The x-axis represents the bleed number, and the y-axis represents the log 10 (IC50) values. C. Distribution of the neutralisation titre of bleed 4 against Ancestral and VOCs—Beta, Gamma, Delta, and BA.1. The x-axis represents the pseudoviruses test for neutralisation, and the y-axis represents the log 10 (IC50) values. The boxplots are colour coded according to vaccines, and appear in the following order from left to right for each challenge variant: WT dER, T2_29, T2_29+Q, and T2_29+Q+dER. D. Distribution of the neutralisation titre of bleed 6 against Ancestral and VOCs—Beta, Gamma, Delta, and BA.1. The x-axis represents the pseudoviruses test for neutralisation, and the y-axis represents the log 10 (IC50) values. The boxplots are colour coded according to vaccines, and the vaccines appear in the same order as for FIG. 50C. Mann-Whitney U test is used as statistical significance test in all the plots (p-value: *≤0.05, **<0.01, ***≤0.001). The distribution that are not statistically significant are not labelled in the plot;

### EXAMPLE 38

### Glycan Masking of a Non-Neutralising Epitope Alters the Balance of Neutralising Antibodies to the Receptor Binding Domain of SARS-CoV-2 and its Variants

### ABSTRACT

Vaccination has saved millions of lives from COVID-19, and millions more from long lasting and poorly understood sequelae. This amazing success has been mediated by robust vaccine platforms using the full length SARS COV-2 spike protein as an immunogen. As expected of RNA viruses, new variants have evolved and quickly replaced the ancestral wild type SARS-CoV-2, leading to escape from natural infection or vaccine induced immunity to the original ancestral SARS-CoV-2 virus. Vaccines that confer specific and targeted immunity to broadly neutralising epitopes on the SARS-CoV-2 spike protein against different SARS COV-2 variants could offer an advancement on current booster shots of previously used vaccines. Here, we present a targeted approach to the elicitation of antibodies that bind and neutralise both SARS-CoV-2, and the variants of concern, by introducing a glycosylation site on a non-neutralising epitope of the RBD. The addition of a glycosylation site in the RBD based vaccine candidate, delivered in a DNA prime- and recombinant viral vector boost using Modified Vaccinia Virus Ankara (MVA) focused the immune response towards broadly neutralising epitopes in comparison to the ancestral SARS CoV-2 RBD vaccine candidate. We further observed enhanced cross-neutralisation and cross-binding using a DNA-MVA prime-boost regime, thus demonstrating the superiority of the glycan engineered RBD vaccine candidate across two platforms and a promising candidate as a ‘variant proof’ booster to SARS-CoV-2 for broader neutralisation capacity.

### INTRODUCTION

Severe acute respiratory syndrome coronavirus 2 (SARS-CoV-2) is the causative agent of COVID-19. Since its emergence in late 2019, SARS-CoV-2 has rapidly spread worldwide, causing mortality and morbidity in all the age groups, but especially the elderly and those with pre-existing health concerns. To date, more than 500 million cases have been reported resulting in around 6.4 million deaths worldwide (https://www.who.int/emergencies/diseases/novel-coronavirus-2019). Much of the deaths and severe manifestation of the disease has been brought down considerably worldwide by rapid and effective introduction of vaccines by the end of 2020. Both the mRNA-based (Moderna and Pfizer/BioNTech) as well as viral vector-based (Oxford-AstraZeneca and Jansen and Jansen) SARS-CoV-2 vaccine candidates induce strong neutralising antibody responses against SARS-CoV-2 and are highly effective at protecting against hospitalisation, severe disease and mortality¹⁻⁸. Most of the currently licensed and approved COVID-19 vaccines are based on the stabilised prefusion conformation of the spike protein derived from the WA-1/2020 strain. The spike protein serves as the most important target antigen as the trimeric spike protein at the virion surface and is essential for virus cell entry^{9,10}. During infection, SARS-CoV-2 uses the receptor-binding domain (RBD) of the spike protein as a key functional component to interact with angiotensin-converting enzyme 2 (ACE-2) on host cells^{11,12}. The trimeric S protein can be in a receptor inaccessible (closed), or accessible (open) state based on the down or up positions respectively of its receptor-binding domain (RBD) (FIG. 54A). Studies have shown that the RBD of SARS-CoV-2 is mainly in the closed conformation which complicates the recognition of the virus particle by the immune system before entering the host cell^{13,14}. The receptor-binding motif (RBM) is the most important motif in the RBD and is composed of two regions that form the interface between the S protein and hACE-2 (FIG. 54B). The RBM is responsible for attachment to the ACE-2 receptor. The region outside the RBM is essential in maintaining the structural stability of the RBD¹⁵. Upon RBD-ACE-2 interaction and spike proteolytic priming by the serine transmembrane protease TMPRSS2, conformational changes lead to the membrane fusion of the spike protein and subsequent entry of the virus into the host cell¹⁵. Antibodies targeting the RBD has been reported to be effective against the infection, making RBD subunit based vaccines a promising candidate for generation of potent and specific neutralising antibodies¹⁶. Furthermore it was clearly shown that the recombinant spike RBD protein of SARS-CoV-2 can potently induce a protective immune response in mice, rabbits, and non-human primates¹⁷. Administration of RBD subunit-based vaccine can also lead to exposure of cryptic epitopes to immune systems, which would otherwise been inaccessible in the full-length spike protein.

At the beginning of the pandemic, evolution of SARS COV-2 was estimated to be slow in line with the evolution rate of other human coronaviruses¹⁸, but since late 2020 several SARS COV-2 variants of concern (VOC) with potentially enhanced transmission, pathogenicity, immune escape, or a combination of all three have emerged and caused multiple waves of infections¹⁹. These SARS CoV-2 variants of concern are characterized by numerous mutations throughout the genome of SARS COV-2, but much of the immune escaping mutation are concentrated in the spike protein, especially the RBD. Therefore, raising constant concerns that they may escape from therapeutic monoclonals and vaccine-induced antibodies. Multiple circulating and evolving lineages now exist, the first of which are designated by the WHO as Alpha, Beta, Gamma, Delta, and Omicron variants²⁰. Most of the mutations in these VOCs have been reported to result in an increased binding affinity to the human ACE-2 receptor²¹. VOC strains currently circulating include those from lineage B.1.1.7 (Alpha), B.1.351 (Beta), P.1 (Gamma), B.1.617.2 (Delta) and B.1.1.529 (Omicron BA.1), first identified in the United Kingdom, South Africa, Brazil, India, and South Africa respectively. B.1.351 and P.1 contain, amongst others, the E484K mutation within the RBD that has been shown to abrogate antibody responses generated from infection or vaccination^{2,22}. B.1.617.2 contains the L452R mutation that contributes to immune evasion in combination with T478K, which leads to the increased transmissibility and immune escape seen with this lineage^{23,24} B.1.1.529 has over 30 mutations in the spike protein, influencing neutralising antibodies generated to previous strains or vaccines, as well as reducing the need for TMPRSS2 priming upon viral attachment and entry²⁵⁻²⁷. A multiple sequence alignment showing these mutations in reference to the Ancestral WA-1/2020 strain is shown in FIG. 54D. In the light of emerging VOCs there is a definite need of a vaccine that can generate a broad neutralising antibody against the known VOCs and could result in a better preparedness for future immune escaping SARS COV-2 mutants.

In this example we present a glycan engineered SARS COV-2 RBD variant based on DNA and generated as a recombinant viral modified vaccinia virus Ankara (MVA) vector vaccine that resulted in a potent binding and neutralising antibody response to all the VOCs tested compared to the SARS COV-2 ancestral RBD in BALB/c mice. The glycan engineered SARS COV-2 RBD variant showed a superior immune response than the ancestral SARS-CoV-2 RBD across two different vaccination regimen such as DNA-DNA and DNA-MVA respectively and vaccination resulted in a protective effect in BALB/c mice after a live challenge using the ancestral SARS COV-2 WA-1/2020 strain. These results obtained from ELISA, pseudotype microneutralization assays and challenge data support the glycan-engineered SARS COV-2 RBD vaccine candidate as a promising candidate for future booster vaccines. Furthermore, in this manuscript we demonstrate that introducing a glycan can focus immune responses towards neutralizing antibodies.

### Results

### Design of the Glycan Engineered SARS COV-2 RBD Antigens—M7 and M8

Glycan engineering of antigenic epitope regions has been shown to focus and facilitate the induction of immune responses to certain epitopes and enhanced the elicitation of neutralising antibodies by either shielding of non-neutralising epitopes or exposing conserved and neutralising epitope regions²⁸. To design novel SARS-CoV-2 RBD modified antigens, three epitope regions of the class 1 monoclonal antibody (mAb) B38²⁹ (FIG. 54A, shown in red brown), class 3 mAb CR3022³⁰ (FIG. 54A, shown in yellow) and class 4 S309³¹ (FIG. 54A, shown in grey) were selected for glycan engineering of the SARS COV-2 RBD ancestral sequence in order to shield or expose certain neutralizing or non-neutralizing epitopes by the addition or removal of N-linked glycosylation sites. The epitope regions of the mAb CR3022 and mAb S309 are outside of the SARS COV-2 receptor binding motif (RBM) which is known to be recognised by many antibodies in convalescent sera from SARS-CoV-2 infected individuals³² while the epitope region of B38 overlaps with the RBM. The CR3022 mAb and S309 mAb have been shown to bind and neutralise SARS-CoV-1 but only the S309 mAb binds and neutralises SARS-CoV-2 while CR3022 only binds SARS-CoV-2³⁰. The S309 epitope has two naturally occurring N-linked glycosylation sites at position 331 and 334 (FIG. 54B), while the CR3022 epitope site is devoid of any glycan. Interestingly, the CR3022 epitope has a glycosylation site in SARS-CoV-1. To understand the effect of glycosylation modifications on the overall immune response to SARS-CoV-2 RBD, two SARS-CoV-2 glycan mutants, namely SARS-CoV-2 RBD M7 (henceforth referred as M7) (amino acid SEQ ID NO:33) and SARS-CoV-2 RBD M8 (henceforth referred as M8) (amino acid sequence SEQ ID NO:34) (FIG. 54B) were engineered. In M7, an additional glycan was added at position 521 located in the epitope region of CR3022 (FIG. 54B). The SARS-CoV-2 RBD M8 was engineered by removing the two natural glycans at position 331 and 334 located in the S309 epitope and addition of a glycan at position 372 that is known to be present in the CR3022 epitope of SARS-CoV-1 (FIG. 54B).

### M7 DNA Based Vaccine Candidate Favourably Tips the Ratio of Neutralising Antibodies to Binding Antibodies Against SARS-CoV-2

For in vitro characterisation of the DNA-based glycan engineered M7 and M8, total cell lysates from HEK293T cells were prepared 48 h after transfection, followed by Western blot analysis. Staining of the membrane with a polyclonal SARS-CoV-2 rabbit antibody showed that all the DNA constructs were successfully expressed at the expected band of approximately of 35 kDa. M7 appears in the immunoblot blot slightly higher due to the addition of a glycan, whereas M8 runs slightly lower due to the removal of glycosylation sites compared to the SARS-CoV-2 RBD WT protein (FIG. 55A). To evaluate the immunogenicity of the DNA vaccine candidates M7 and M8 in comparison to the ancestral SARS-CoV-2, BALB/c mice (n=6) were vaccinated with 50 μg of the DNA vaccine construct expressing M7, M8 or wild type SARS COV-2 RBD subcutaneously four times at two-week intervals (FIG. 55B). An overview of the SARS-CoV-2 RBD DNA vaccine constructs including the mutations for each construct are provided in Table 13. Blood samples were collected every two weeks and analysed for both binding antibodies (bAb) and neutralising (nAb) using SARS-CoV-2 RBD based direct ELISA and pseudovirus neutralisation assay against SARS-CoV-2, respectively.

After the first DNA immunisation there was no significant difference observed in the levels of bAb and nAb titres induced by the M7 and SARS-CoV-2 RBD WT vaccine construct (FIG. 55C), whereas M8 elicited weaker bAb and nAb responses in comparison to both M7 and WT SARS CoV-2 RBD (FIG. 55C). Interestingly, after the fourth and last DNA immunisation, mice immunised with M7 generated slightly lower but not statistically different levels of bAb than the WT SARS-CoV-2 RBD and comparable nAb (FIG. 55D). M8 generated substantially lower nAb and bAb in comparison to WT SARS-CoV-2 RBD but comparable bAb to M7. These observation of different ratios of bAb and nAb between M7, M8, and WT SARS-CoV-2 RBD (FIG. 55E), suggest that masking of the CR3022 epitope via the addition of a glycan at position 521 induces a greater proportion of neutralising antibodies for a given bAb titre while the de-masking of S309 epitope by removing the glycan position at 331 and 343 and simultaneous introduction of glycan at position 372, reduces both the bAb as well as nAb. Taken together, the SARS-CoV-2 RBD WT construct induced homologous bAbs, whereas the SARS-CoV-2 RBD M7 was capable to elicit heterologous bAbs and therefore to focus and direct immune response to the neutralising epitopes through shielding of the CR3022 epitope. However, the M8 construct elicited weaker bAbs and nAbs and was excluded from further studies.

TABLE 13
Glycan engineered SARS-CoV-2 RBD DNA vaccine
constructs evaluated in this study.

| DNA vaccine construct | Mutations |
|---|---|
| pURVac SARS-CoV-2 RBD WT | — |
| pURVAc SARS-CoV-2 RBD M7 | P521N |
| pURVac SARS-CoV-2 RBD M8 | N331Q |
| | N343Q |
| | A372T |

### Design, Generation, and Biochemical Characterisation of Recombinant MVAs Expressing M7 and WT SARS-CoV-2 RBD

Since MVA as a recombinant viral vector is known to effectively boost DNA-primed specific immune responses against multiple infectious diseases^{33,34}, recombinant MVAs were generated encoding the SARS-CoV-2 WT RBD and M7. The sequences of SARS-CoV-2 RBD and SARS-CoV-2 RBD M7 were cloned into the MVA transfer vector pMVA Trans TK under the control of a synthetic poxviral early/late promoter mH5, respectively. The antigens were integrated into the TK locus of the CR19 MVA genome via homologous recombination using MVA CR19 TK GFP as a starting viral vector for fluorescent selection of recombinant MVAs (FIG. 56A). The recombinant MVAs were generated on the AGE1.CR.pIX cell line and purified over several plaque purification rounds until a pure recombinant MVA was obtained. The MVA seed stock was purified via ultracentrifugation through a sucrose cushion gradient. The expression of the antigens was tested in vitro by Western blot analysis. HEK293T cells were infected with the MVA CR19 TK SARS-CoV-2 WT RBD and MVA CR19 TK M7 at a MOI of 2 and 24 h post infection total cell lysates were prepared and subjected to Western blot analysis. The immunoblot stained with a polyclonal SARS-CoV-2 S specific rabbit antibody revealed good antigen expression of both recombinant MVAs with a band around 35 kDa for MVA CR19 TK SARS-CoV-2 RBD WT and a slightly larger band for the glycan engineered MVA CR19 TK M7 (FIG. 56B).

### M7 DNA Prime Followed by a MVA Boost Induces Higher and Longer Lasting Cross-Reactive Titres Binding and Neutralizing Antibodies Against VOCs

To evaluate whether a heterologous DNA prime/MVA boost regimen can induce higher, broadly neutralising, and long-lasting antibodies against VOCs, BALB/c mice (n=6) were immunised subcutaneously with 50 μg of DNA vaccines encoding SARS-CoV-2 RBD WT and SARS-CoV-2 RBD M7 on day 0. At week 2, the mice were either vaccinated with a heterologous MVA boost using MVA SARS-CoV-2 RBD WT and MVA SARS-CoV-2 RBD M7 at with a dose 2×107 pfu per animal intramuscularly or immunised subcutaneously with 50 μg of DNA vaccines encoding SARS-CoV-2 RBD WT and SARS-CoV-2 RBD M7. The bleeds were collected 2 weeks after each immunisation until week 10. For longitudinal analysis of binding and neutralising antibodies, the terminal bleed was taken at week 11 (FIG. 57A). For analysing the binding antibodies against SARS-CoV-2 RBD VOCs, sera from individual mice collected at week 20 were analysed by direct RBD ELISA against Wuhan-1 B, Alpha B.1.1.7, Beta B.1.351, Gamma P.1, Delta B.1.617.2, and Omicron BA.1 RBD. Interestingly, the titres of anti-SARS COV-2 RBD binding antibodies at week 16 were higher across all the tested circulating VOCs in mice that received a boost with MVA in comparison to DNA boost. Similar binding profiles were observed across the VOCs, except Omicron BA.1 RBD. Comparing MVA SARS-CoV-2 RBD WT and RBD M7 across all VOCs, RBD M7 is superior to RBD WT in the induction of binding antibodies across all SARS-CoV-2 RBD VOC (FIG. 57B).

To measure the impact of a heterologous DNA prime/MVA boost immunisation on the induction of higher, long lasting, broadly neutralising antibody, mice sera from week 16 were evaluated against Wuhan-1 B, Alpha B.1.1.7, Beta B.1.351, Gamma P.1, Delta B.1.617.2 and Omicron BA.1 using lentiviral pseudotype microneutralisation assays. The neutralising antibody response also followed the same trend as the binding antibody levels measured by direct RBD ELISA with a significant increase for mice that received a heterologous MVA boost versus mice that were vaccinated two times with DNA vaccine (FIG. 57C). The strongest nAb response could be observed in MVA RBD M7 boosted mice against Wuhan-1 B, Alpha B.1.1.7, Gamma P.1, Delta B.1.617.2 variants. The neutralisation titres against Beta B.1.351 and Omicron BA.1 were much reduced but still relatively high in the mice that were vaccinated with a heterologous MVA boost. On comparing the induction of neutralising antibodies by MVA RBD WT versus RBD M7, the neutralisation titres were observed to significantly lower for MVA RBD WT against Wuhan-1 B, Alpha B.1.1.7, Gamma P.1, Delta B.1.617.2, whereas the difference for Beta B.1.351 and Omicron BA.1 and BA.2 bearing pseudoviruses was not significant.

In conclusion, SARS-CoV-2 RBD M7 DNA prime followed by a MVA boost was superior to two times DNA immunisation and induced higher and longer lasting cross-reactive titres binding and neutralising antibodies against all circulating VOCs and were still relatively high even 7 weeks after the MVA boost.

Human ACE2 Transduced Mice Challenged with SARS-CoV-2 Live Virus

To investigate whether a homologous SARS-CoV-2 RBD M7 DNA prime/DNA boost or heterologous SARS-CoV-2 RBD M7 DNA prime/MVA boost regimen can provide protection from SARS-CoV-2 wild type live virus, a challenge study using human ACE2 transduced BALB/c mice was carried out. For immunisation one group of BALB/c mice (n=12) received two doses of 50 μg of the SARS-CoV-2 M7 DNA vaccine subcutaneously, whereas another group of BALB/c mice (n=12) was vaccinated using a heterologous SARS-CoV-2 RBD M7 DNA prime/MVA boost vaccination regimen with 2×10⁷ pfu (plaque forming unit) intramuscularly at day 0 and week 2. The study was set up longitudinally and sera were collected 2 weeks after each immunization, followed by week 16 and the terminal bleed at week 19 (FIG. 58A).

Two weeks post MVA boost the binding antibodies specific to SARS-CoV-2 and its' variants were analysed by direct RBD ELISA. The binding antibodies were measured against all VOC RBDs including Wuhan-1 B, Alpha B.1.1.7, Beta B.1.351, Gamma P.1, Delta B.1.617.2 and Omicron BA.1 (FIG. 58B). In mice that received M7 MVA boost, the binding antibody titres across all VOCs were significantly higher compared to the mice that were vaccinated two times with M7 DNA. In the MVA boosted mice the binding antibody titres across all VOC RBDs were very high with AUC values above 4, except for Omicron BA.1 that showed AUC values of around 1-2. The neutralisation titres were extremely high and higher in mice that received a heterologous MVA boost compared to mice that were vaccinated two times with DNA (FIG. 58C). The neutralisation was measured against Wuhan-1 B, Alpha B.1.1.7, Beta B.1.351, Gamma P.1, Delta B.1.617.2 and Omicron BA.1 two weeks after the MVA boost.

Prior to challenge with live virus, BALB/c mice were transduced with 1×10⁷ pfu of the ad5-huACE2 vector five days before infection with SARS-CoV-2. For infection with SARS-CoV-2 live virus BALB/c mice received 1×10⁴ pfu of Australia/VIC01/2020 (SARS-CoV-2 B) by intranasal route. The challenge was carried out 14 weeks post last immunisation (FIG. 58A).

Dna-MVA Prime-Boost Regime Results in a Reduction of Viral Load after Challenge with SARS CoV-2 Wildtype Strain

To investigate the durability and waning immunity over time, sera from the longitudinal challenge study were analysed for their binding and neutralising capacity across all variants. After the prime immunisation with DNA, bAb responses were detected in 7/12 mice in the DNA/DNA group whereas 9/12 mice in the DNA/MVA group showed binding antibodies against SARS-CoV-2 (FIG. 59A). The neutralising antibody response against SARS-CoV-2 was low after priming with DNA (FIG. 59B). After the boost with either DNA or MVA, the binding and neutralising antibodies increased significantly with MVA providing a significantly higher boost than DNA at week 4 after boost (FIGS. 59A and 59B). Two weeks after the boost, bAb and nAb responses were significantly higher in the MVA boosted group as compared to the DNA boosted group (FIGS. 59A and 59B). Over the course of the 19-week study, nAb responses peaked four weeks after the boost (week 6) and declined in both DNA/DNA and DNA/MVA groups until the pre-challenge bleed (week 16 FIGS. 59A and 59B).

Mice were rendered susceptible to SARS-CoV-2 by intranasal administration of Ad5-huACE-2 construct and challenged five days later with SARS-CoV-2 Australia/VIC01/2020. Due to the lack of published disease readouts in wild type mice at the time of challenge, even after Ad5-huACE-2 transduction, the decision was made to cull mice at days 3 and 6 post infection to measure virus replication in the lungs. An increase in nAb titres was observed in the terminal bleed sera, in line with a typical reaction to encountering the virus. Terminal sera from culled mice were also tested against VOCs Beta, Gamma, Delta, and Omicron, with a subset of mice showing decreases in or abrogation of nAb, as expected based on the published literature, particularly to Gamma and Omicron (FIG. 59C). Intriguingly, binding, and neutralising antibodies across all VOCs could be detected 14 weeks after the last immunisation suggesting that the MVA boost induces strong, broad, and longer lasting neutralising antibody response that resulted in a reduction of viral load in the lungs after challenge. In contrast, the mice that received two times DNA did not show any reduction of SARS-CoV-2 lung genome copies when compared to naïve controls (FIG. 59D).

Challenged mice showed a positive correlation between detected nAb and bAb responses (r2=0.44, P=<0.0001 Despite weak or no neutralising responses in subsets of mice in all groups at the time of challenge, or the terminal bleed 3-6 days after challenge, a strong inverse correlation was observed between copies of SARS-CoV-2 in the lungs of infected mice and their respective nAb or bAb antibody titre (Pearson's r2=0.49 and 0.62 respectively, P=<0.0001), see FIG. 59E confirming a direct link between RBD-directed neutralising antibodies and the reduction of SARS-CoV-2 replication in the lungs.

While true protection from disease or infection could not be proven with this non-lethal animal model, these results confirm that antibodies and specifically nAb have an antagonistic effect on the virus, and that these antibodies are present in mice immunised with the glycan engineered M7 vaccine.

### Discussion

With the emergence of new variants of SARS-CoV-2 that can escape immunity generated either with prior infection or vaccination, it is imperative that we need a periodic regime of boosters to stay ahead of the variants. In addition to emergence of variants, another concern is the waning of the immunity against SARS-CoV-2 over the time. Given these, a next generation vaccine candidate should provide a better coverage to known as well as emerging variants and longer lasting immunity. Here, we discuss a novel glycan engineered RBD based vaccine antigen (M7) that generated better neutralising response in comparison to wild-type (WT) SARS-CoV-2 RBD. The novel antigen has a single point mutation in comparison to the WT, which introduced a unique glycosylation site in the construct. The glycosylation site was introduced in such a way that it would mask an epitope that was reported to generate non-neutralising antibody, for example CR3022. This was done in line with the assumption that neutralising antibodies would be a better correlate of protection than the non-neutralising but binding antibodies. As per our design strategy, M7 indeed generated a higher proportion of neutralising antibody for the given titre of binding antibody in comparison to WT, when given in DNA prime-boost regime. We also generated another glycosylation site modified construct (M8), where we altered the glycosylation pattern of one of the neutralising epitopes and one non-neutralising epitope. After four successive immunisations, M8 and M7 generated a similar titre of binding antibodies but substantially different levels of neutralising antibodies. This observation strongly suggests the de-glycosylation of the neutralising epitope leads to an inferior vaccine construct.

To further interrogate the superiority of M7 in comparison to WT SARS COV-2 and applicability of the design strategy across platforms, we tested and compared the immunogenicity of M7 in a DNA-DNA versus a DNA-MVA prime-boost regime. MVA has already been established as an excellent booster following DNA prime^{33,34}. DNA-MVA prime-boost regime induces significantly higher titres of binding and neutralising antibodies in comparison to DNA-DNA prime-boost regime and for longer duration in comparison to DNA-DNA prime-boost regime. M7 in DNA-MVA prime-boost regime show better neutralisation of all the VOCs. Among the VOCs, we observed minimal neutralisation against Omicron. This observation is in line with already published data on diminished immune responses against Omicron. Based on all these observations, we propose that the better neutralisation ability against VOCs by M7 is due to the higher proportion of the neutralising antibodies in comparison to WT SARS-CoV-2. A reduced viral load in human ACE2 transduced mice is observed but further work is needed to interrogate the difference between WT and M7 RBDs in their ability to protect mice from challenge.

Overall, the data presented here strongly supports the superiority of the M7 vaccine antigen over the WT SARS-CoV-2 across two vaccination platforms such as DNA-DNA and DNA vector and DNA-MVA prime-boost regime. The observation of the alteration of the neutralising and binding antibody titres by introduction of glycosylation site provides evidence for general applicability of glycosylation modification of the vaccine antigens to tweak the immune response to epitopes of interest and broaden immune response.

### Supplementary Information

### FIG. 54 and FIG. 66

(A) Surface representation of the Wuhan-1 B.1 SARS COV-2 RBD protein in the “up” conformation. Representative epitopes that were selected for glycan engineering of the SARS COV-2 RBD are colored in red-brown (B38, class 1 mAb), yellow (CR3022, class 4 mAb) and grey (S309, class 3 mAb).

(B) Schematic illustration and multiple sequence alignment of glycan engineered SARS COV-2 RBD mutants. The SARS COV-2 RBD located in S1 (depicted in blue) contains two N-glycosylation sites at position 331 and 334 (indicated with glycosylation molecule). In the SARS COV-2 RBD M7 an additional glycan was added at position 521 downstream of the receptor binding motif (RBM, depicted in red). The SARS COV-2 RBD M8 was designed in a way that the glycans at position 331 and 334 located in the S309 epitope were removed and an additional SARS COV-1 glycan of the CR3022 epitope was introduced at position 372. In the panel below a multiple sequence alignment of all the SARS COV-2 RBD mutants is depicted. FIG. 60A shows an enlarged version of the sequence alignment.

(C) Surface representation of glycan engineered SARS COV-2 RBD mutants. In the SARS COV-2 RBD WT protein the glycosylation sites at positions 331 and 343 are shown as green spheres. In the SARS COV-2 RBD M7 mutant an additional glycosylation site at position 521 was added in order to shield the CR3022 epitope (yellow), whereas in the SARS COV-2 RBD M8 mutant the glycans at positions 331 and 343 were removed and an additional SARS COV-1 glycan was introduced at position 372.

(D) Multiple sequence alignment of the SARS COV-2 WT RBD and the VOCs. FIG. 60B shows an enlarged version of the sequence alignment.

### FIG. 55

(A) Expression analysis of DNA based vaccine candidates encoding glycan engineered SARS CoV-2 RBD mutants in vitro. Western blot analysis of HEK293T cell lysates transfected with DNA vectors expressing SARS COV-2 RBD mutants and controls. The cells were harvested after 48 hours. Antigens were detected using a polyclonal SARS COV-2 spike-specific antibody (top panel). As loading control the membrane was stained with a monoclonal anti-Tubulin antibody (bottom panel). Size in kilodaltons (kDa) and size of the molecular weight marker are indicated.

(B) Immunization schedule of Balb/c mice vaccinated with DNA-based vaccines encoding SARS CoV-2 RBD mutants. In the top panel a representation of all tested DNA vaccines is shown. For immunogenicity analysis Balb/c mice (n=6) were vaccinated subcutaneously with a dose of 50 μg four times in a two weeks interval with DNA vectors encoding for SARS COV-2 RBD WT (depicted in purple), SARS COV-2 RBD M7 (shown in magenta) or SARS COV-2 RBD M8 (colored in dark blue). The bleeds were taken two weeks after each immunization. Mice were sacrificed at week 12.

(C) Humoral immune response induced just after one immunization with the SARS COV-2 RBD M7 DNA vaccine candidate. Titers of binding antibodies specific for SARS COV-2 RBD (Wuhan strain) were analysed by ELISA in individual mouse serum samples 2 weeks after the first DNA immunization (left panel). The antibody binding titers are represented as area under the curve (AUC) values in a box plot. The neutralization titers (shown in the right hand panel) were evaluated two weeks after the first DNA immunization against SARS COV-2 WT (Wuhan strain) using lentiviral pseudotype microneutralisation assays. The neutralization titers are shown as log IC50 values in a box plot. For statistical analysis the unpaired Mann-Whitney test was used and *p<0.05; **p<0.005 as asteriks or ns for non-significant was indicated.

(D) Binding and neutralizing antibodies induced after 4 immunisations with the SARS COV-2 RBD M7 DNA vaccine candidate. Antibody binding to SARS COV-2 RBD was determined in individual mouse serum samples by ELISA two weeks after the last DNA immunization and represented as AUC values in a box plot format (left hand panel). Pseudovirus neutralization titers against SARS CoV-2 WT (Wuhan strain) 2 weeks after the last DNA immunization are shown on the right hand panel as log IC50 values in a box plot. The Mann-Whitney statistical test was applied and *p<0.05; **p<0.005 as asteriks or ns for non-significant was indicated.

(E) Binding and neutralizing antibodies induced after 1 immunisation with the SARS COV-2 RBD M7 and M8 DNA vaccine candidate (bleed 2; week 2).

(F) Binding and neutralizing antibodies induced after 4 immunisations with the SARS COV-2 RBD M7 and M8 DNA vaccine candidate (bleed 5; week 8).

### FIG. 56

(A) Schematic representation of the MVA genome and design of the recombinant SARS COV-2 RBD WT and SARS COV-2 RBD M7 MVAs. The MVA genome consists of the left terminal region, the central conserved region and right conserved region and includes major deletion sites. The J2R region or TK locus was used to insert the antigens for SARS COV-2 RBD WT and SARS COV-2 RBD M7 via homologous recombination between MVA DNA sequences (TK-L and TK-R) and the shuttle vector pMVA Trans mH5 TK SARS COV-2 RBD WT and SARS COV-2 RBD M7, respectively. Antigen expression is controlled by the strong early/late poxviral promoter mH5. The recombinant MVAs were generated on the AGE1.CR.pIX cell line through several rounds of plaque purifications and ultracentrifugation via sucrose cushion.

(B) Expression analysis of recombinant MVAs encoding SARS COV-2 RBD WT and SARS COV-2 M7 RBD. Western blot analysis of HEK293T cell lysates infected with a MOI of 2.0 and harvested after 24 h. For antigen detection a polyclonal SARS COV-2 spike-specific antibody (top panel) and as a loading control a monoclonal anti-Tubulin antibody (bottom panel) was used. Size in kilodaltons (kDa) and corresponding bands of the protein standard are indicated.

### FIG. 57

(A) Immunization schedule of Balb/c mice vaccinated using different DNA prime/MVA boost regimen. Balb/c mice (n=6) were primed at day 0 with either SARS COV-2 RBD WT or SARS COV-2 RBD M7 vaccine receiving 50 μg subcutaneously. Two weeks later Balb/c mice were boosted with 2×107 pfu (plaque forming unit) intramuscularly. Sera were collected two weeks after each immunization. For long term analysis of binding/neutralizing antibodies Balb/c mice were sacrificed at week 20.

(B) Titers of anti-SARS COV-2 RBD binding antibodies were determined by ELISA at week 20. Binding antibodies were measured against all VOC RBDs including Wuhan-1 B.1, Alpha B.1.1.7, Beta B.1.351, Gamma P.1, Delta B.1.617.2 and Omicron BA.1 and represented as AUC values.

(C) Neutralization titers against all circulating VOCs to date were evaluated in mouse sera collected at week 20. The neutralization was determined against Wuhan-1 B.1, Alpha B.1.1.7, Beta B.1.351, Gamma P.1, Delta B.1.617.2 and Omicron BA.1. The neutralization titers are shown as log IC50 values.

### FIG. 58

(A) Immunization schedule of BALB/c mice vaccinated using different DNA prime/MVA boost regimen followed by a challenge with SARS COV-2 live virus. BALB/c mice (n=6) were primed at day 0 with either SARS COV-2 RBD WT or SARS COV-2 RBD M7 vaccine receiving 50 μg subcutaneously. Two weeks later Balb/c mice were boosted with 2×107 pfu (plaque forming unit) intramuscularly. Sera were collected two weeks after each immunization until week 12. For challenge, BALB/c mice were transduced with 1×107 pfu of the ad5-huACE2 vector five days before infection with SARS-CoV-2. For infection with SARS COV-2 live virus BALB/c mice received 1×104 pfu of Australia/VIC01/2020 (SARS-CoV-2 B.1) by intranasal route.

(B) Binding antibodies specific to SARS COV-2 and ist variants were analyzed by ELISA two weeks after the boost with either DNA or MVA. Binding antibodies were measured against all VOC RBDs including Wuhan-1 B.1, Alpha B.1.1.7, Beta B.1.351, Gamma P.1, Delta B.1.617.2 and Omicron BA.1 and represented as AUC values.

(C) Neutralization titers against all circulating VOCs to date were evaluated in mouse sera taken two weeks after the boost with either DNA or MVA. The neutralization was measured against Wuhan-1 B.1, Alpha B.1.1.7, Beta B.1.351, Gamma P.1, Delta B.1.617.2 and Omicron BA.1. The neutralization titers are shown as log IC50 values.

### FIG. 59

(A) Titers of anti-SARS COV-2 RBD binding antibodies were measured by ELISA using sera collected at weeks 2, 4 and week 20 from challenged mice. The binding antibodies were determined against the SARS COV-2 WT RBD and represented as AUC values.

(B) Neutralization titers against lentiviral SARS COV-2 pseudotypes were evaluated from mouse sera at week 2, 4 and the terminal bleed at week 20. The neutralization titers are shown as log IC50 values.

(C) Neutralization titers against all circulating VOCs to date were evaluated in mouse sera collected at week 20. The neutralization was measured against Wuhan-1 B.1, Alpha B.1.1.7, Beta B.1.351, Gamma P.1, Delta B.1.617.2 and Omicron BA.1. The neutralization titers are shown as log IC50 values.

(D) SARS-CoV-2 genome copies from the lungs of infected mice at day 3 (D3) and day 6 (D6) post infection shown as log 10 copies/gram of lung.

(E) Correlation of binding (AUC) (left panel) and neutralising (IC50) antibody titer (shown in the middle panel) and SARS-CoV-2 genome copies per gram of lung. On the right hand panel the correlation of binding in AUC and neutralizing antibodies as log IC50 values in immunised mice is shown.

### REFERENCES

1. Lopez Bernal, J. et al. Effectiveness of Covid-19 Vaccines against the B.1.617.2 (Delta) Variant. N Engl J Med NEJMoa2108891 (2021) doi: 10.1056/NEJMoa2108891.
2. Madhi, S. A. et al. Efficacy of the ChAdOx1 nCOV-19 Covid-19 Vaccine against the B.1.351 Variant. New England Journal of Medicine 1-14 (2021) doi: 10.1056/nejmoa2102214.
3. Lopez Bernal, J. et al. Effectiveness of the Pfizer-BioNTech and Oxford-AstraZeneca vaccines on covid-19 related symptoms, hospital admissions, and mortality in older adults in England: test negative case-control study. BMJ 373, n1088 (2021).
4. Baden, L. R. et al. Efficacy and Safety of the mRNA-1273 SARS-CoV-2 Vaccine. New England Journal of Medicine 384, 403-416 (2021).
5. Sadoff, J. et al. Safety and Efficacy of Single-Dose Ad26.COV2.S Vaccine against Covid-19. N Engl J Med 384, 2187-2201 (2021).
6. Polack, F. P. et al. Safety and Efficacy of the BNT162b2 mRNA Covid-19 Vaccine. New England Journal of Medicine 383, 2603-2615 (2020).
7. Voysey, M. et al. Safety and efficacy of the ChAdOx1 nCOV-19 vaccine (AZD1222) against SARS-CoV-2: an interim analysis of four randomised controlled trials in Brazil, South Africa, and the UK. The Lancet 397, 99-111 (2021).
8. Self, W. H. et al. Comparative Effectiveness of Moderna, Pfizer-BioNTech, and Janssen (Johnson & Johnson) Vaccines in Preventing COVID-19 Hospitalizations Among Adults Without Immunocompromising Conditions-United States, March-August 2021. MMWR Morb Mortal Wkly Rep 70, 1337-1343 (2021).
9. Letko, M., Marzi, A. & Munster, V. Functional assessment of cell entry and receptor usage for SARS-CoV-2 and other lineage B betacoronaviruses. Nature Microbiology 5, 562-569 (2020).
10. Walls, A. C. et al. Structure, Function, and Antigenicity of the SARS-CoV-2 Spike Glycoprotein. Cell 181, 281-292.e6 (2020).
11. Fehr, A. R. & Perlman, S. Coronaviruses: An Overview of Their Replication and Pathogenesis. Coronaviruses 1282, 1-23 (2015).
12. Yan, R. et al. Structural basis for the recognition of SARS-CoV-2 by full-length human ACE2. Science 367, 1444-1448 (2020).
13. Carnell, G. W. et al. SARS-CoV-2 Spike Protein Stabilized in the Closed State Induces Potent Neutralizing Responses. Journal of Virology 95, e00203-21.
14. Xiong, X. et al. A thermostable, closed SARS-CoV-2 spike protein trimer. Nature Structural & Molecular Biology 27, 934-941 (2020).
15. Hoffmann, M. et al. SARS-CoV-2 Cell Entry Depends on ACE2 and TMPRSS2 and Is Blocked by a Clinically Proven Protease Inhibitor. Cell 181, 271-280.e8 (2020).
16. Du, L., Yang, Y. & Zhang, X. Neutralizing antibodies for the prevention and treatment of COVID-19. Cell Mol Immunol 18, 2293-2306 (2021).
17. Yang, J. et al. A vaccine targeting the RBD of the S protein of SARS-CoV-2 induces protective immunity. Nature 586, 572-577 (2020).
18. Jo, W. K., Drosten, C. & Drexler, J. F. The evolutionary dynamics of endemic human coronaviruses. Virus Evol 7, veab020 (2021).
19. Singh, J., Pandit, P., McArthur, A. G., Banerjee, A. & Mossman, K. Evolutionary trajectory of SARS-CoV-2 and emerging variants. Virol J 18, 166 (2021).
20. WHO announces simple, easy-to-say labels for SARS-CoV-2 Variants of Interest and Concern. https://www.who.int/news/item/31-05-2021-who-announces-simple-easy-to-say-labels-for-sars-cov-2-variants-of-interest-and-concern.
21. Montagutelli, X. et al. The B1.351 and P.1 variants extend SARS-CoV-2 host range to mice. bioRxiv 2021.03.18.436013 (2021) doi: 10.1101/2021.03.18.436013.
22. Kustin, T. et al. Evidence for increased breakthrough rates of SARS-CoV-2 variants of concern in BNT162b2 mRNA vaccinated individuals. Clalit Health Services 2021.04.06.21254882 (2021) doi: 10.1101/2021.04.06.21254882.
23. Li, Q. et al. The Impact of Mutations in SARS-CoV-2 Spike on Viral Infectivity and Antigenicity. Cell 182, 1284-1294.e9 (2020).
24. Callaway, E. Delta coronavirus variant: scientists brace for impact. Nature 595, 17-18 (2021).
25. Martin, D. P. et al. Selection analysis identifies unusual clustered mutational changes in Omicron lineage BA.1 that likely impact Spike function. 2022.01.14.476382 Preprint at https://doi.org/10.1101/2022.01.14.476382 (2022).
26. Meng, B. et al. Altered TMPRSS2 usage by SARS-CoV-2 Omicron impacts infectivity and fusogenicity. Nature 603, 706-714 (2022).
27. Viana, R. et al. Rapid epidemic expansion of the SARS-CoV-2 Omicron variant in southern Africa. Nature (2022) doi: 10.1038/s41586-022-04411-y.
28. Du, L. et al. Introduction of neutralizing immunogenicity index to the rational design of MERS coronavirus subunit vaccines. Nat Commun 7, 13473 (2016).
29. Wu, Y. et al. A noncompeting pair of human neutralizing antibodies block COVID-19 virus binding to its receptor ACE2. Science 368, 1274-1278 (2020).
30. Yuan, M. et al. A highly conserved cryptic epitope in the receptor binding domains of SARS-CoV-2 and SARS-CoV. Science 368, 630-633 (2020).
31. Pinto, D. et al. Cross-neutralization of SARS-CoV-2 by a human monoclonal SARS-CoV antibody. Nature 583, 290-295 (2020).
32. Barnes, C. O. et al. SARS-CoV-2 neutralizing antibody structures inform therapeutic strategies. Nature 588, 682-687 (2020).
33. Asbach, B. et al. Priming with a Potent HIV-1 DNA Vaccine Frames the Quality of Immune Responses prior to a Poxvirus and Protein Boost. Journal of Virology 93, e01529-18 (2019).
34. Richert, L. et al. T Cell Immunogenicity, Gene Expression Profile, and Safety of Four Heterologous Prime-Boost Combinations of HIV Vaccine Candidates in Healthy Volunteers: Results of the Randomized Multi-Arm Phase I/II ANRS VRI01 Trial. The Journal of Immunology (2022) doi:10.4049/jimmunol.2101076.

### EXAMPLE 39

This example describes DNA vaccine constructs according to embodiments of the invention. In particular, the example describes the amino acid sequence of COV_S_T2_17+tPA (tPA signal peptide sequence), and its encoding nucleic acid sequence. Also described are the nucleic acid sequences for embodiments of the invention COV_S_T2_29, CoV_S_T2_29+Q498R, and CoV_S_T2_29+Q498R+dER. The example further describes the nucleic acid sequences of pURVAC DNA vector comprising designed nucleic acid sequences according to the invention. Also shown is successful transfection of HEK293T cells with pURVAC COV_S_T2_17+tPA and CoV_S_T2_29+Q498R+dER DNA constructs, and subsequent expression of the encoded antigen sequences.

### Cov_S_T2_17 and Related Constructs

The amino acid sequence and encoding nucleic acid sequence for COV_S_T2_17+tPA is given below. The tPA signal sequence is highlighted in grey. pURVAC-COV_S_T2_17+tPA is also provided.

>CoV_S_T2_17 + tPA signal peptide
Amino acid sequence
(SEQ ID NO: 92)
MDAMKRGLCCVLLLCGAVFVSPSAARVAPTKEVVRFPNITNLCPFGEVFNATKEPSVYAWERKKISNC
VADYSVLYNSTSFSTFKCYGVSPTKLNDLCFTNVYADSFVIRGDEVRQIAPGQTGVIADYNYKLPDDF
TGCVIAWNTNNIDSTTGGNYNYLYRSLRKSKLKPFERDISSDIYSPGGKPCSGVEGFNCYYPLRSYGF
FPTNGTGYQPYRVVVLSFELLNAPATVCGPKLSTD
>CoV_S_T2_17 + tPA signal peptide
Nucleic acid sequence
(SEQ ID NO: 93)
ATGGATGCTATGAAGAGGGGCCTGTGCTGCGTGCTGCTTCTGTGTGGCGCTGTGTTTGTGTCTCCTAG
CGCCGCTAGAGTGGCCCCTACCAAAGAAGTCGTGCGGTTCCCCAACATCACCAATCTGTGCCCTTTCG
GCGAGGTGTTCAACGCCACCAAGTTTCCCTCTGTGTACGCCTGGGAGCGCAAAAAGATCAGCAACTGC
GTGGCCGACTACAGCGTGCTGTACAACAGCACCAGCTTCAGCACCTTCAAGTGCTACGGCGTGTCACC
CACCAAGCTGAACGACCTGTGCTTCACCAACGTGTACGCCGACAGCTTCGTGATCAGAGGCGACGAAG
TGCGGCAGATTGCCCCTGGACAAACAGGCGTGATCGCCGATTACAACTACAAGCTGCCCGACGACTTC
ACCGGCTGTGTGATCGCCTGGAACACCAACAACATCGACAGCACCACCGGCGGCAACTACAACTACCT
GTACAGAAGCCTGCGGAAGTCTAAGCTGAAGCCCTTCGAGCGGGACATCAGCAGCGACATCTATAGCC
CTGGCGGCAAGCCTTGTTCTGGCGTGGAAGGCTTCAACTGCTACTACCCTCTGCGGAGCTACGGCTTC
TTCCCCACAAATGGCACAGGCTACCAGCCTTACAGAGTGGTGGTCCTGAGCTTCGAGCTGCTGAATGC
CCCTGCCACAGTGTGTGGCCCTAAGCTGTCTACCGACTGA
>pURVAC + CoV_S_T2_17 + tPA signal peptide
Nucleic acid sequence
(SEQ ID NO: 94)
TCGCGCGTTTCGGTGATGACGGTGAAAACCTCTGACACATGCAGCTCCCGGAGACGGTCACAGCTTGT
CTGTAAGCGGATGCCGGGAGCAGACAAGCCCGTCAGGGCGCGTCAGCGGGTGTTGGCGGGTGTCGGGG
CTGGCTTAACTATGCGGCATCAGAGCAGATTGTACTGAGAGTGCACCATATGCGGTGTGAAATACCGC
ACAGATGCGTAAGGAGAAAATACCGCATCAGATTGGCTATTGGCCATTGCATACGTTGTATCCATATC
ATAATATGTACATTTATATTGGCTCATGTCCAACATTACCGCCATGTTGACATTGATTATTGACTAGT
TATTAATAGTAATCAATTACGGGGTCATTAGTTCATAGCCCATATATGGAGTTCCGCGTTACATAACT
TACGGTAAATGGCCCGCCTGGCTGACCGCCCAACGACCCCCGCCCATTGACGTCAATAATGACGTATG
TTCCCATAGTAACGCCAATAGGGACTTTCCATTGACGTCAATGGGTGGAGTATTTACGGTAAACTGCC
CACTTGGCAGTACATCAAGTGTATCATATGCCAAGTACGCCCCCTATTGACGTCAATGACGGTAAATG
GCCCGCCTGGCATTATGCCCAGTACATGACCTTATGGGACTTTCCTACTTGGCAGTACATCTACGTAT
TAGTCATCGCTATTACCATGGTGATGCGGTTTTGGCAGTACATCAATGGGCGTGGATAGCGGTTTGAC
TCACGGGGATTTCCAAGTCTCCACCCCATTGACGTCAATGGGAGTTTGTTTTGGCACCAAAATCAACG
GGACTTTCCAAAATGTCGTAACAACTCCGCCCCATTGACGCAAATGGGCGGTAGGCGTGTACGGTGGG
AGGTCTATATAAGCAGAGCTCGTTTAGTGAACCGTCAGATCGCCTGGAGACGCCATCCACGCTGTTTT
GACCTCCATAGAAGACACCGGGACCGATCCAGCCTCCATCGGCTCGCATCTCTCCTTCACGCGCCCGC
CGCCCTACCTGAGGCCGCCATCCACGCCGGTTGAGTCGCGTTCTGCCGCCTCCCGCCTGTGGTGCCTC
CTGAACTGCGTCCGCCGTCTAGGTAAGTTTAAAGCTCAGGTCGAGACCGGGCCTTTGTCCGGCGCTCC
CTTGGAGCCTACCTAGACTCAGCCGGCTCTCCACGCTTTGCCTGACCCTGCTTGCTCAACTCTAGTTA
ACGGTGGAGGGCAGTGTAGTCTGAGCAGTACTCGTTGCTGCCGCGCGCGCCACCAGACATAATAGCTG
ACAGACTAACAGACTGTTCCTTTCCATGGGTCTTTTCTGCAGTCACCGTCGGTACCGCCACCATGGAT
GCTATGAAGAGGGGCCTGTGCTGCGTGCTGCTTCTGTGTGGCGCTGTGTTTGTGTCTCCTAGCGCCGC
TAGAGTGGCCCCTACCAAAGAAGTCGTGCGGTTCCCCAACATCACCAATCTGTGCCCTTTCGGCGAGG
TGTTCAACGCCACCAAGTTTCCCTCTGTGTACGCCTGGGAGCGCAAAAAGATCAGCAACTGCGTGGCC
GACTACAGCGTGCTGTACAACAGCACCAGCTTCAGCACCTTCAAGTGCTACGGCGTGTCACCCACCAA
GCTGAACGACCTGTGCTTCACCAACGTGTACGCCGACAGCTTCGTGATCAGAGGCGACGAAGTGCGGC
AGATTGCCCCTGGACAAACAGGCGTGATCGCCGATTACAACTACAAGCTGCCCGACGACTTCACCGGC
TGTGTGATCGCCTGGAACACCAACAACATCGACAGCACCACCGGCGGCAACTACAACTACCTGTACAG
AAGCCTGCGGAAGTCTAAGCTGAAGCCCTTCGAGCGGGACATCAGCAGCGACATCTATAGCCCTGGCG
GCAAGCCTTGTTCTGGCGTGGAAGGCTTCAACTGCTACTACCCTCTGCGGAGCTACGGCTTCTTCCCC
ACAAATGGCACAGGCTACCAGCCTTACAGAGTGGTGGTCCTGAGCTTCGAGCTGCTGAATGCCCCTGC
CACAGTGTGTGGCCCTAAGCTGTCTACCGACTGAGCGGCCGCAGATCTGCTGTGCCTTCTAGTTGCCA
GCCATCTGTTGTTTGCCCCTCCCCCGTGCCTTCCTTGACCCTGGAAGGTGCCACTCCCACTGTCCTTT
CCTAATAAAATGAGGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGATGCGGTGGGCTCTAT
GGCTACCCAGGTGCTGAAGAATTGACCCGGTTCCTCCTGGGCCAGAAAGAAGCAGGCACATCCCCTTC
TCTGTGACACACCCTGTCCACGCCCCTGGTTCTTAGTTCCAGCCCCACTCATAGGACACTCATAGCTC
AGGAGGGCTCCGCCTTCAATCCCACCCGCTAAAGTACTTGGAGCGGTCTCTCCCTCCCTCATCAGCCC
ACCAAACCAAACCTAGCCTCCAAGAGTGGGAAGAAATTAAAGCAAGATAGGCTATTAAGTGCAGAGGG
AGAGAAAATGCCTCCAACATGTGAGGAAGTAATGAGAGAAATCATAGAATTTTAAGGCCATGATTTAA
GGCCATCATGGCCTTAATCTTCCGCTTCCTCGCTCACTGACTCGCTGCGCTCGGTCGTTCGGCTGCGG
CGAGCGGTATCAGCTCACTCAAAGGCGGTAATACGGTTATCCACAGAATCAGGGGATAACGCAGGAAA
GAACATGTGAGCAAAAGGCCAGCAAAAGGCCAGGAACCGTAAAAAGGCCGCGTTGCTGGCGTTTTTCC
ATAGGCTCCGCCCCCCTGACGAGCATCACAAAAATCGACGCTCAAGTCAGAGGTGGCGAAACCCGACA
GGACTATAAAGATACCAGGCGTTTCCCCCTGGAAGCTCCCTCGTGCGCTCTCCTGTTCCGACCCTGCC
GCTTACCGGATACCTGTCCGCCTTTCTCCCTTCGGGAAGCGTGGCGCTTTCTCATAGCTCACGCTGTA
GGTATCTCAGTTCGGTGTAGGTCGTTCGCTCCAAGCTGGGCTGTGTGCACGAACCCCCCGTTCAGCCC
GACCGCTGCGCCTTATCCGGTAACTATCGTCTTGAGTCCAACCCGGTAAGACACGACTTATCGCCACT
GGCAGCAGCCACTGGTAACAGGATTAGCAGAGCGAGGTATGTAGGCGGTGCTACAGAGTTCTTGAAGT
GGTGGCCTAACTACGGCTACACTAGAAGAACAGTATTTGGTATCTGCGCTCTGCTGAAGCCAGTTACC
TTCGGAAAAAGAGTTGGTAGCTCTTGATCCGGCAAACAAACCACCGCTGGTAGCGGTGGTTTTTTTGT
TTGCAAGCAGCAGATTACGCGCAGAAAAAAAGGATCTCAAGAAGATCCTTTGATCTTTTCTACGGGGT
CTGACGCTCAGTGGAACGAAAACTCACGTTAAGGGATTTTGGTCATGAGATTATCAAAAAGGATCTTC
ACCTAGATCCTTTTAAATTAAAAATGAAGTTTTAAATCAATCTAAAGTATATATGAGTAAACTTGGTC
TGACAGTTACCAATGCTTAATCAGTGAGGCACCTATCTCAGCGATCTGTCTATTTCGTTCATCCATAG
TTGCCTGACTCGGGGGGGGGGGGCGCTGAGGTCTGCCTCGTGAAGAAGGTGTTGCTGACTCATACCAG
GCCTGAATCGCCCCATCATCCAGCCAGAAAGTGAGGGAGCCACGGTTGATGAGAGCTTTGTTGTAGGT
GGACCAGTTGGTGATTTTGAACTTTTGCTTTGCCACGGAACGGTCTGCGTTGTCGGGAAGATGCGTGA
TCTGATCCTTCAACTCAGCAAAAGTTCGATTTATTCAACAAAGCCGCCGTCCCGTCAAGTCAGCGTAA
TGCTCTGCCAGTGTTACAACCAATTAACCAATTCTGATTAGAAAAACTCATCGAGCATCAAATGAAAC
TGCAATTTATTCATATCAGGATTATCAATACCATATTTTTGAAAAAGCCGTTTCTGTAATGAAGGAGA
AAACTCACCGAGGCAGTTCCATAGGATGGCAAGATCCTGGTATCGGTCTGCGATTCCGACTCGTCCAA
CATCAATACAACCTATTAATTTCCCCTCGTCAAAAATAAGGTTATCAAGTGAGAAATCACCATGAGTG
ACGACTGAATCCGGTGAGAATGGCAAAAGCTTATGCATTTCTTTCCAGACTTGTTCAACAGGCCAGCC
ATTACGCTCGTCATCAAAATCACTCGCATCAACCAAACCGTTATTCATTCGTGATTGCGCCTGAGCGA
GACGAAATACGCGATCGCTGTTAAAAGGACAATTACAAACAGGAATCGAATGCAACCGGCGCAGGAAC
ACTGCCAGCGCATCAACAATATTTTCACCTGAATCAGGATATTCTTCTAATACCTGGAATGCTGTTTT
CCCGGGGATCGCAGTGGTGAGTAACCATGCATCATCAGGAGTACGGATAAAATGCTTGATGGTCGGAA
GAGGCATAAATTCCGTCAGCCAGTTTAGTCTGACCATCTCATCTGTAACATCATTGGCAACGCTACCT
TTGCCATGTTTCAGAAACAACTCTGGCGCATCGGGCTTCCCATACAATCGATAGATTGTCGCACCTGA
TTGCCCGACATTATCGCGAGCCCATTTATACCCATATAAATCAGCATCCATGTTGGAATTTAATCGCG
GCCTCGAGCAAGACGTTTCCCGTTGAATATGGCTCATAACACCCCTTGTATTACTGTTTATGTAAGCA
GACAGTTTTATTGTTCATGATGATATATTTTTATCTTGTGCAATGTAACATCAGAGATTTTGAGACAC
AACGTGGCTTTCCCCCCCCCCCCATTATTGAAGCATTTATCAGGGTTATTGTCTCATGAGCGGATACA
TATTTGAATGTATTTAGAAAAATAAACAAATAGGGGTTCCGCGCACATTTCCCCGAAAAGTGCCACCT
GACGTCTAAGAAACCATTATTATCATGACATTAACCTATAAAAATAGGCGTATCACGAGGCCCTTTCG
TC

### CoV_S_T2_29 And Related Constructs

The nucleic acid sequences for constructs relating to CoV_S_T2_29 are given below, including T2_29, T2_29+Q498R, and vaccine candidate COV_S_T2_29+Q498R+dER (the amino acid sequences are provided in Example 30 and 37 below). The Example also provides the nucleic acid sequence of pURVAC+COV_S_T2_29+Q498R+dER.

>CoV_S_T2_29
Nucleic acid sequence
(SEQ ID NO: 89)
ATGTTCGTGTTTCTGGTGCTGCTGCCTCTGGTGTCCAGCCAGTGCGTGAACTTCACCAAC
AGAACCCAGCTGCCTAGCGCCTACACCAACAGCTTTACCAGAGGCGTGTACTACCCCGAC
AAGGTGTTCAGATCCAGCGTGCTGCACTCTACCCAGGACCTGTTCCTGCCTTTCTTCAGC
AACGTGACCTGGTTCCACGCCATCAGCGGCACCAATGGCACCAAGAGATTCGACAACCCC
GTGCTGCCCTTCAACGACGGGGTGTACTTTGCCAGCACCGAGAAGTCCAACATCATCAGA
GGCTGGATCTTCGGCACCACACTGGACAGCAAGACCCAGAGCCTGCTGATCGTGAACAAC
GCCACCAACGTGGTCATCAAAGTGTGCGAGTTCCAGTTCTGCAACGACCCCTTCCTGGGC
GTGTACCACAAGAACAACAAGAGCTGGATGGAAAGCGAGTTCCGGGTGTACAGCAGCGCC
AACAACTGCACCTTCGAGTACGTGTCCCAGCCTTTCCTGATGGACCTGGAAGGCAAGCAG
GGCAACTTCAAGAACCTGCGCGAGTTCGTGTTCAAGAACATCGACGGCTACTTCAAAATC
TACAGCAAGCACACCCCTATCAACCTCGTGCGGGATCTGCCTCAGGGCTTCTCTGCTCTG
GAACCCCTGGTGGATCTGCCCATCGGCATCAACATCACCCGGTTTCAGACACTGCTGGCC
CTGCACAGAAGCTACCTGACACCTGGCGATAGCAGCAGCGGATGGACAGCTGGTGCCGCC
GCTTACTACGTGGGATACCTCCAGCCTAGAACCTTCCTGCTGAAGTACAACGAGAACGGC
ACCATCACCGACGCCGTGGATTGTGCTCTGGACCCTCTGAGCGAGACAAAGTGCACCCTG
AAGTCCTTCACCGTGGAAAAGGGCATCTACCAGACCAGCAACTTCCGGGTGCAGCCCACC
GAATCCATCGTGCGGTTCCCCAATATCACCAATCTGTGCCCCTTCGGCGAGGTGTTCAAT
GCCACCAGATTCGCCTCTGTGTACGCCTGGAACCGGAAGCGGATCAGCAATTGCGTGGCC
GACTACTCCGTGCTGTACAACTCCGCCAGCTTCAGCACCTTCAAGTGCTACGGCGTGTCC
CCTACCAAGCTGAACGACCTGTGCTTCACAAACGTGTACGCCGACAGCTTCGTGATCCGG
GGAGATGAAGTGCGGCAGATTGCCCCTGGACAGACCGGCAATATCGCCGACTACAACTAC
AAGCTGCCCGACGACTTCACCGGCTGTGTGATTGCCTGGAACAGCAACAACCTGGACTCC
AAAGTCGGCGGCAACTACAATTACCTGTACCGGCTGTTCCGGAAGTCCAATCTGAAGCCC
TTCGAGCGGGACATCAGCACCGAAATCTATCAGGCCGGCAGCACCCCTTGCAATGGCGTG
AAGGGCTTTAACTGCTACTTCCCACTGCAAAGCTACGGCTTCCAGCCAACATACGGCGTG
GGCTACCAGCCTTACAGAGTGGTGGTGCTGAGCTTCGAGCTGCTGCATGCTCCTGCCACA
GTGTGCGGCCCTAAGAAATCCACCAATCTCGTGAAGAACAAATGCGTCAACTTCAATTTC
AACGGCCTGACCGGCACCGGCGTGCTGACAGAGAGCAACAAGAAGTTCCTGCCATTCCAG
CAGTTCGGCCGGGACATTGCCGATACCACAGATGCCGTCAGAGATCCCCAGACACTGGAA
ATCCTGGACATCACCCCATGCAGCTTCGGCGGAGTGTCTGTGATCACCCCTGGCACCAAC
ACCAGCAATCAGGTGGCAGTGCTGTACCAGGGCGTCAACTGTACAGAGGTGCCAGTGGCC
ATTCACGCCGATCAGCTGACCCCTACTTGGCGGGTGTACTCCACAGGCAGCAATGTGTTT
CAGACCAGAGCCGGCTGTCTGATCGGAGCCGAGCACGTGAACAATAGCTACGAGTGCGAC
ATCCCCATCGGCGCTGGCATCTGCGCCTCTTACCAGACACAGACCAACAGCCACAGACGG
GCTAGAAGCGTGGCCAGCCAGAGCATCATTGCCTACACAATGTCTCTGGGCGCCGAGAAC
AGCGTGGCCTACTCCAACAACTCTATCGCTATCCCCACCAATTTCACCATCAGCGTGACC
ACCGAGATCCTGCCTGTGTCCATGACCAAGACCAGCGTGGACTGCACCATGTACATCTGC
GGCGATTCCACCGAGTGCTCCAACCTGCTGCTCCAGTACGGCAGCTTCTGCACCCAGCTG
AATAGAGCCCTGACAGGGATCGCCGTGGAACAGGACAAGAACACCCAAGAGGTGTTCGCC
CAAGTGAAGCAAATCTACAAGACCCCTCCTATCAAGGACTTCGGCGGCTTCAACTTCAGC
CAGATTCTGCCCGATCCTAGCAAGCCCAGCAAGCGGAGCTTCATCGAGGACCTGCTGTTC
AACAAAGTGACACTGGCCGACGCCGGCTTCATCAAGCAGTACGGCGATTGTCTGGGCGAC
ATTGCAGCCAGGGATCTGATCTGCGCCCAGAAGTTTAACGGACTGACAGTGCTGCCTCCT
CTGCTGACCGATGAGATGATCGCCCAGTACACATCTGCCCTGCTGGCCGGCACAATCACA
AGCGGCTGGACATTTGGAGCTGGCGCCGCTCTCCAGATTCCATTCGCTATGCAGATGGCC
TACAGGTTCAACGGCATCGGAGTGACCCAGAATGTGCTGTACGAGAACCAGAAGCTGATC
GCCAACCAGTTCAACAGCGCCATCGGCAAGATCCAGGACAGCCTGAGCAGCACAGCAAGC
GCCCTGGGAAAGCTCCAGGACGTGGTCAACCAGAATGCCCAGGCACTGAACACCCTGGTC
AAGCAGCTGTCCTCCAACTTCGGCGCCATCTCTAGCGTGCTGAACGATATCCTGAGCAGA
CTGGACCCACCTGAGGCCGAGGTGCAGATCGACAGACTGATCACAGGCCGGCTCCAGAGC
CTCCAGACATACGTTACACAGCAGCTGATCAGAGCCGCCGAGATTAGAGCCTCTGCCAAT
CTGGCCGCCACCAAGATGTCTGAGTGTGTGCTGGGCCAGAGCAAGAGAGTGGACTTTTGC
GGCAAGGGCTACCACCTGATGAGCTTCCCTCAGTCTGCACCACACGGCGTGGTGTTTCTG
CACGTGACATACGTGCCCGCTCAAGAGAAGAACTTTACCACCGCTCCAGCCATCTGCCAC
GACGGCAAAGCCCACTTTCCTAGAGAAGGCGTGTTCGTGTCCAACGGCACCCATTGGTTC
GTGACCCAGCGGAACTTCTACGAGCCCCAGATCATCACCACCGACAACACCTTCGTGTCC
GGCAACTGCGACGTCGTGATCGGCATTGTGAACAATACCGTGTACGACCCTCTCCAGCCG
GAACTGGACTCCTTCAAAGAGGAACTGGATAAGTACTTTAAGAACCACACAAGCCCCGAC
GTGGACCTGGGCGATATCAGCGGAATCAATGCCAGCGTCGTGAACATCCAGAAAGAGATC
GACCGGCTGAACGAGGTGGCCAAGAATCTGAACGAGAGCCTGATCGACCTGCAAGAACTG
GGGAAGTACGAGCAGTACATCAAGTGGCCTTGGTACATCTGGCTGGGCTTTATCGCCGGA
CTGATTGCCATCGTGATGGTCACAATCATGCTGTGTTGCATGACCAGCTGTTGCAGCTGC
CTGAAGGGCTGCTGTAGCTGTGGCTCCTGCTGCAAGTTCGACGAGGACGATTCTGAGCCC
GTGCTGAAGGGCGTGAAACTGCACTACACC
>CoV_S_T2_29 + Q498R
Nucleic acid sequence
(SEQ ID NO: 90)
ATGTTCGTGTTTCTGGTGCTGCTGCCTCTGGTGTCCAGCCAGTGCGTGAACTTCACCAAC
AGAACCCAGCTGCCTAGCGCCTACACCAACAGCTTTACCAGAGGCGTGTACTACCCCGAC
AAGGTGTTCAGATCCAGCGTGCTGCACTCTACCCAGGACCTGTTCCTGCCTTTCTTCAGC
AACGTGACCTGGTTCCACGCCATCAGCGGCACCAATGGCACCAAGAGATTCGACAACCCC
GTGCTGCCCTTCAACGACGGGGTGTACTTTGCCAGCACCGAGAAGTCCAACATCATCAGA
GGCTGGATCTTCGGCACCACACTGGACAGCAAGACCCAGAGCCTGCTGATCGTGAACAAC
GCCACCAACGTGGTCATCAAAGTGTGCGAGTTCCAGTTCTGCAACGACCCCTTCCTGGGC
GTGTACCACAAGAACAACAAGAGCTGGATGGAAAGCGAGTTCCGGGTGTACAGCAGCGCC
AACAACTGCACCTTCGAGTACGTGTCCCAGCCTTTCCTGATGGACCTGGAAGGCAAGCAG
GGCAACTTCAAGAACCTGCGCGAGTTCGTGTTCAAGAACATCGACGGCTACTTCAAAATC
TACAGCAAGCACACCCCTATCAACCTCGTGCGGGATCTGCCTCAGGGCTTCTCTGCTCTG
GAACCCCTGGTGGATCTGCCCATCGGCATCAACATCACCCGGTTTCAGACACTGCTGGCC
CTGCACAGAAGCTACCTGACACCTGGCGATAGCAGCAGCGGATGGACAGCTGGTGCCGCC
GCTTACTACGTGGGATACCTCCAGCCTAGAACCTTCCTGCTGAAGTACAACGAGAACGGC
ACCATCACCGACGCCGTGGATTGTGCTCTGGACCCTCTGAGCGAGACAAAGTGCACCCTG
AAGTCCTTCACCGTGGAAAAGGGCATCTACCAGACCAGCAACTTCCGGGTGCAGCCCACC
GAATCCATCGTGCGGTTCCCCAATATCACCAATCTGTGCCCCTTCGGCGAGGTGTTCAAT
GCCACCAGATTCGCCTCTGTGTACGCCTGGAACCGGAAGCGGATCAGCAATTGCGTGGCC
GACTACTCCGTGCTGTACAACTCCGCCAGCTTCAGCACCTTCAAGTGCTACGGCGTGTCC
CCTACCAAGCTGAACGACCTGTGCTTCACAAACGTGTACGCCGACAGCTTCGTGATCCGG
GGAGATGAAGTGCGGCAGATTGCCCCTGGACAGACCGGCAATATCGCCGACTACAACTAC
AAGCTGCCCGACGACTTCACCGGCTGTGTGATTGCCTGGAACAGCAACAACCTGGACTCC
AAAGTCGGCGGCAACTACAATTACCTGTACCGGCTGTTCCGGAAGTCCAATCTGAAGCCC
TTCGAGCGGGACATCAGCACCGAAATCTATCAGGCCGGCAGCACCCCTTGCAATGGCGTG
AAGGGCTTTAACTGCTACTTCCCACTGCAAAGCTACGGCTTCcggCCAACATACGGCGTG
GGCTACCAGCCTTACAGAGTGGTGGTGCTGAGCTTCGAGCTGCTGCATGCTCCTGCCACA
GTGTGCGGCCCTAAGAAATCCACCAATCTCGTGAAGAACAAATGCGTCAACTTCAATTTC
AACGGCCTGACCGGCACCGGCGTGCTGACAGAGAGCAACAAGAAGTTCCTGCCATTCCAG
CAGTTCGGCCGGGACATTGCCGATACCACAGATGCCGTCAGAGATCCCCAGACACTGGAA
ATCCTGGACATCACCCCATGCAGCTTCGGCGGAGTGTCTGTGATCACCCCTGGCACCAAC
ACCAGCAATCAGGTGGCAGTGCTGTACCAGGGCGTCAACTGTACAGAGGTGCCAGTGGCC
ATTCACGCCGATCAGCTGACCCCTACTTGGCGGGTGTACTCCACAGGCAGCAATGTGTTT
CAGACCAGAGCCGGCTGTCTGATCGGAGCCGAGCACGTGAACAATAGCTACGAGTGCGAC
ATCCCCATCGGCGCTGGCATCTGCGCCTCTTACCAGACACAGACCAACAGCCACAGACGG
GCTAGAAGCGTGGCCAGCCAGAGCATCATTGCCTACACAATGTCTCTGGGCGCCGAGAAC
AGCGTGGCCTACTCCAACAACTCTATCGCTATCCCCACCAATTTCACCATCAGCGTGACC
ACCGAGATCCTGCCTGTGTCCATGACCAAGACCAGCGTGGACTGCACCATGTACATCTGC
GGCGATTCCACCGAGTGCTCCAACCTGCTGCTCCAGTACGGCAGCTTCTGCACCCAGCTG
AATAGAGCCCTGACAGGGATCGCCGTGGAACAGGACAAGAACACCCAAGAGGTGTTCGCC
CAAGTGAAGCAAATCTACAAGACCCCTCCTATCAAGGACTTCGGCGGCTTCAACTTCAGC
CAGATTCTGCCCGATCCTAGCAAGCCCAGCAAGCGGAGCTTCATCGAGGACCTGCTGTTC
AACAAAGTGACACTGGCCGACGCCGGCTTCATCAAGCAGTACGGCGATTGTCTGGGCGAC
ATTGCAGCCAGGGATCTGATCTGCGCCCAGAAGTTTAACGGACTGACAGTGCTGCCTCCT
CTGCTGACCGATGAGATGATCGCCCAGTACACATCTGCCCTGCTGGCCGGCACAATCACA
AGCGGCTGGACATTTGGAGCTGGCGCCGCTCTCCAGATTCCATTCGCTATGCAGATGGCC
TACAGGTTCAACGGCATCGGAGTGACCCAGAATGTGCTGTACGAGAACCAGAAGCTGATC
GCCAACCAGTTCAACAGCGCCATCGGCAAGATCCAGGACAGCCTGAGCAGCACAGCAAGC
GCCCTGGGAAAGCTCCAGGACGTGGTCAACCAGAATGCCCAGGCACTGAACACCCTGGTC
AAGCAGCTGTCCTCCAACTTCGGCGCCATCTCTAGCGTGCTGAACGATATCCTGAGCAGA
CTGGACCCACCTGAGGCCGAGGTGCAGATCGACAGACTGATCACAGGCCGGCTCCAGAGC
CTCCAGACATACGTTACACAGCAGCTGATCAGAGCCGCCGAGATTAGAGCCTCTGCCAAT
CTGGCCGCCACCAAGATGTCTGAGTGTGTGCTGGGCCAGAGCAAGAGAGTGGACTTTTGC
GGCAAGGGCTACCACCTGATGAGCTTCCCTCAGTCTGCACCACACGGCGTGGTGTTTCTG
CACGTGACATACGTGCCCGCTCAAGAGAAGAACTTTACCACCGCTCCAGCCATCTGCCAC
GACGGCAAAGCCCACTTTCCTAGAGAAGGCGTGTTCGTGTCCAACGGCACCCATTGGTTC
GTGACCCAGCGGAACTTCTACGAGCCCCAGATCATCACCACCGACAACACCTTCGTGTCC
GGCAACTGCGACGTCGTGATCGGCATTGTGAACAATACCGTGTACGACCCTCTCCAGCCG
GAACTGGACTCCTTCAAAGAGGAACTGGATAAGTACTTTAAGAACCACACAAGCCCCGAC
GTGGACCTGGGCGATATCAGCGGAATCAATGCCAGCGTCGTGAACATCCAGAAAGAGATC
GACCGGCTGAACGAGGTGGCCAAGAATCTGAACGAGAGCCTGATCGACCTGCAAGAACTG
GGGAAGTACGAGCAGTACATCAAGTGGCCTTGGTACATCTGGCTGGGCTTTATCGCCGGA
CTGATTGCCATCGTGATGGTCACAATCATGCTGTGTTGCATGACCAGCTGTTGCAGCTGC
CTGAAGGGCTGCTGTAGCTGTGGCTCCTGCTGCAAGTTCGACGAGGACGATTCTGAGCCC
GTGCTGAAGGGCGTGAAACTGCACTACACC
>CoV_S_T2_29 + Q498R + dER
Nucleic acid sequence
(SEQ ID NO: 91)
ATGTTCGTGTTTCTGGTGCTGCTGCCTCTGGTGTCCAGCCAGTGCGTGAACTTCACCAAC
AGAACCCAGCTGCCTAGCGCCTACACCAACAGCTTTACCAGAGGCGTGTACTACCCCGAC
AAGGTGTTCAGATCCAGCGTGCTGCACTCTACCCAGGACCTGTTCCTGCCTTTCTTCAGC
AACGTGACCTGGTTCCACGCCATCAGCGGCACCAATGGCACCAAGAGATTCGACAACCCC
GTGCTGCCCTTCAACGACGGGGTGTACTTTGCCAGCACCGAGAAGTCCAACATCATCAGA
GGCTGGATCTTCGGCACCACACTGGACAGCAAGACCCAGAGCCTGCTGATCGTGAACAAC
GCCACCAACGTGGTCATCAAAGTGTGCGAGTTCCAGTTCTGCAACGACCCCTTCCTGGGC
GTGTACCACAAGAACAACAAGAGCTGGATGGAAAGCGAGTTCCGGGTGTACAGCAGCGCC
AACAACTGCACCTTCGAGTACGTGTCCCAGCCTTTCCTGATGGACCTGGAAGGCAAGCAG
GGCAACTTCAAGAACCTGCGCGAGTTCGTGTTCAAGAACATCGACGGCTACTTCAAAATC
TACAGCAAGCACACCCCTATCAACCTCGTGCGGGATCTGCCTCAGGGCTTCTCTGCTCTG
GAACCCCTGGTGGATCTGCCCATCGGCATCAACATCACCCGGTTTCAGACACTGCTGGCC
CTGCACAGAAGCTACCTGACACCTGGCGATAGCAGCAGCGGATGGACAGCTGGTGCCGCC
GCTTACTACGTGGGATACCTCCAGCCTAGAACCTTCCTGCTGAAGTACAACGAGAACGGC
ACCATCACCGACGCCGTGGATTGTGCTCTGGACCCTCTGAGCGAGACAAAGTGCACCCTG
AAGTCCTTCACCGTGGAAAAGGGCATCTACCAGACCAGCAACTTCCGGGTGCAGCCCACC
GAATCCATCGTGCGGTTCCCCAATATCACCAATCTGTGCCCCTTCGGCGAGGTGTTCAAT
GCCACCAGATTCGCCTCTGTGTACGCCTGGAACCGGAAGCGGATCAGCAATTGCGTGGCC
GACTACTCCGTGCTGTACAACTCCGCCAGCTTCAGCACCTTCAAGTGCTACGGCGTGTCC
CCTACCAAGCTGAACGACCTGTGCTTCACAAACGTGTACGCCGACAGCTTCGTGATCCGG
GGAGATGAAGTGCGGCAGATTGCCCCTGGACAGACCGGCAATATCGCCGACTACAACTAC
AAGCTGCCCGACGACTTCACCGGCTGTGTGATTGCCTGGAACAGCAACAACCTGGACTCC
AAAGTCGGCGGCAACTACAATTACCTGTACCGGCTGTTCCGGAAGTCCAATCTGAAGCCC
TTCGAGCGGGACATCAGCACCGAAATCTATCAGGCCGGCAGCACCCCTTGCAATGGCGTG
AAGGGCTTTAACTGCTACTTCCCACTGCAAAGCTACGGCTTCcggCCAACATACGGCGTG
GGCTACCAGCCTTACAGAGTGGTGGTGCTGAGCTTCGAGCTGCTGCATGCTCCTGCCACA
GTGTGCGGCCCTAAGAAATCCACCAATCTCGTGAAGAACAAATGCGTCAACTTCAATTTC
AACGGCCTGACCGGCACCGGCGTGCTGACAGAGAGCAACAAGAAGTTCCTGCCATTCCAG
CAGTTCGGCCGGGACATTGCCGATACCACAGATGCCGTCAGAGATCCCCAGACACTGGAA
ATCCTGGACATCACCCCATGCAGCTTCGGCGGAGTGTCTGTGATCACCCCTGGCACCAAC
ACCAGCAATCAGGTGGCAGTGCTGTACCAGGGCGTCAACTGTACAGAGGTGCCAGTGGCC
ATTCACGCCGATCAGCTGACCCCTACTTGGCGGGTGTACTCCACAGGCAGCAATGTGTTT
CAGACCAGAGCCGGCTGTCTGATCGGAGCCGAGCACGTGAACAATAGCTACGAGTGCGAC
ATCCCCATCGGCGCTGGCATCTGCGCCTCTTACCAGACACAGACCAACAGCCACAGACGG
GCTAGAAGCGTGGCCAGCCAGAGCATCATTGCCTACACAATGTCTCTGGGCGCCGAGAAC
AGCGTGGCCTACTCCAACAACTCTATCGCTATCCCCACCAATTTCACCATCAGCGTGACC
ACCGAGATCCTGCCTGTGTCCATGACCAAGACCAGCGTGGACTGCACCATGTACATCTGC
GGCGATTCCACCGAGTGCTCCAACCTGCTGCTCCAGTACGGCAGCTTCTGCACCCAGCTG
AATAGAGCCCTGACAGGGATCGCCGTGGAACAGGACAAGAACACCCAAGAGGTGTTCGCC
CAAGTGAAGCAAATCTACAAGACCCCTCCTATCAAGGACTTCGGCGGCTTCAACTTCAGC
CAGATTCTGCCCGATCCTAGCAAGCCCAGCAAGCGGAGCTTCATCGAGGACCTGCTGTTC
AACAAAGTGACACTGGCCGACGCCGGCTTCATCAAGCAGTACGGCGATTGTCTGGGCGAC
ATTGCAGCCAGGGATCTGATCTGCGCCCAGAAGTTTAACGGACTGACAGTGCTGCCTCCT
CTGCTGACCGATGAGATGATCGCCCAGTACACATCTGCCCTGCTGGCCGGCACAATCACA
AGCGGCTGGACATTTGGAGCTGGCGCCGCTCTCCAGATTCCATTCGCTATGCAGATGGCC
TACAGGTTCAACGGCATCGGAGTGACCCAGAATGTGCTGTACGAGAACCAGAAGCTGATC
GCCAACCAGTTCAACAGCGCCATCGGCAAGATCCAGGACAGCCTGAGCAGCACAGCAAGC
GCCCTGGGAAAGCTCCAGGACGTGGTCAACCAGAATGCCCAGGCACTGAACACCCTGGTC
AAGCAGCTGTCCTCCAACTTCGGCGCCATCTCTAGCGTGCTGAACGATATCCTGAGCAGA
CTGGACCCACCTGAGGCCGAGGTGCAGATCGACAGACTGATCACAGGCCGGCTCCAGAGC
CTCCAGACATACGTTACACAGCAGCTGATCAGAGCCGCCGAGATTAGAGCCTCTGCCAAT
CTGGCCGCCACCAAGATGTCTGAGTGTGTGCTGGGCCAGAGCAAGAGAGTGGACTTTTGC
GGCAAGGGCTACCACCTGATGAGCTTCCCTCAGTCTGCACCACACGGCGTGGTGTTTCTG
CACGTGACATACGTGCCCGCTCAAGAGAAGAACTTTACCACCGCTCCAGCCATCTGCCAC
GACGGCAAAGCCCACTTTCCTAGAGAAGGCGTGTTCGTGTCCAACGGCACCCATTGGTTC
GTGACCCAGCGGAACTTCTACGAGCCCCAGATCATCACCACCGACAACACCTTCGTGTCC
GGCAACTGCGACGTCGTGATCGGCATTGTGAACAATACCGTGTACGACCCTCTCCAGCCG
GAACTGGACTCCTTCAAAGAGGAACTGGATAAGTACTTTAAGAACCACACAAGCCCCGAC
GTGGACCTGGGCGATATCAGCGGAATCAATGCCAGCGTCGTGAACATCCAGAAAGAGATC
GACCGGCTGAACGAGGTGGCCAAGAATCTGAACGAGAGCCTGATCGACCTGCAAGAACTG
GGGAAGTACGAGCAGTACATCAAGTGGCCTTGGTACATCTGGCTGGGCTTTATCGCCGGA
CTGATTGCCATCGTGATGGTCACAATCATGCTGTGTTGCATGACCAGCTGTTGCAGCTGC
CTGAAGGGCTGCTGTAGCTGTGGCTCCTGCTGCTGA
>pURVAC + CoV_S_T2_29 + Q498R + dER
Nucleic acid sequence
(SEQ ID NO: 95)
TCGCGCGTTTCGGTGATGACGGTGAAAACCTCTGACACATGCAGCTCCCGGAGACGGTCACAGCTTGT
CTGTAAGCGGATGCCGGGAGCAGACAAGCCCGTCAGGGCGCGTCAGCGGGTGTTGGCGGGTGTCGGGG
CTGGCTTAACTATGCGGCATCAGAGCAGATTGTACTGAGAGTGCACCATATGCGGTGTGAAATACCGC
ACAGATGCGTAAGGAGAAAATACCGCATCAGATTGGCTATTGGCCATTGCATACGTTGTATCCATATC
ATAATATGTACATTTATATTGGCTCATGTCCAACATTACCGCCATGTTGACATTGATTATTGACTAGT
TATTAATAGTAATCAATTACGGGGTCATTAGTTCATAGCCCATATATGGAGTTCCGCGTTACATAACT
TACGGTAAATGGCCCGCCTGGCTGACCGCCCAACGACCCCCGCCCATTGACGTCAATAATGACGTATG
TTCCCATAGTAACGCCAATAGGGACTTTCCATTGACGTCAATGGGTGGAGTATTTACGGTAAACTGCC
CACTTGGCAGTACATCAAGTGTATCATATGCCAAGTACGCCCCCTATTGACGTCAATGACGGTAAATG
GCCCGCCTGGCATTATGCCCAGTACATGACCTTATGGGACTTTCCTACTTGGCAGTACATCTACGTAT
TAGTCATCGCTATTACCATGGTGATGCGGTTTTGGCAGTACATCAATGGGCGTGGATAGCGGTTTGAC
TCACGGGGATTTCCAAGTCTCCACCCCATTGACGTCAATGGGAGTTTGTTTTGGCACCAAAATCAACG
GGACTTTCCAAAATGTCGTAACAACTCCGCCCCATTGACGCAAATGGGCGGTAGGCGTGTACGGTGGG
AGGTCTATATAAGCAGAGCTCGTTTAGTGAACCGTCAGATCGCCTGGAGACGCCATCCACGCTGTTTT
GACCTCCATAGAAGACACCGGGACCGATCCAGCCTCCATCGGCTCGCATCTCTCCTTCACGCGCCCGC
CGCCCTACCTGAGGCCGCCATCCACGCCGGTTGAGTCGCGTTCTGCCGCCTCCCGCCTGTGGTGCCTC
CTGAACTGCGTCCGCCGTCTAGGTAAGTTTAAAGCTCAGGTCGAGACCGGGCCTTTGTCCGGCGCTCC
CTTGGAGCCTACCTAGACTCAGCCGGCTCTCCACGCTTTGCCTGACCCTGCTTGCTCAACTCTAGTTA
ACGGTGGAGGGCAGTGTAGTCTGAGCAGTACTCGTTGCTGCCGCGCGCGCCACCAGACATAATAGCTG
ACAGACTAACAGACTGTTCCTTTCCATGGGTCTTTTCTGCAGTCACCGTCGGTACCGCCACCATGTTC
GTGTTTCTGGTGCTGCTGCCTCTGGTGTCCAGCCAGTGCGTGAACTTCACCAACAGAACCCAGCTGCC
TAGCGCCTACACCAACAGCTTTACCAGAGGCGTGTACTACCCCGACAAGGTGTTCAGATCCAGCGTGC
TGCACTCTACCCAGGACCTGTTCCTGCCTTTCTTCAGCAACGTGACCTGGTTCCACGCCATCAGCGGC
ACCAATGGCACCAAGAGATTCGACAACCCCGTGCTGCCCTTCAACGACGGGGTGTACTTTGCCAGCAC
CGAGAAGTCCAACATCATCAGAGGCTGGATCTTCGGCACCACACTGGACAGCAAGACCCAGAGCCTGC
TGATCGTGAACAACGCCACCAACGTGGTCATCAAAGTGTGCGAGTTCCAGTTCTGCAACGACCCCTTC
CTGGGCGTGTACCACAAGAACAACAAGAGCTGGATGGAAAGCGAGTTCCGGGTGTACAGCAGCGCCAA
CAACTGCACCTTCGAGTACGTGTCCCAGCCTTTCCTGATGGACCTGGAAGGCAAGCAGGGCAACTTCA
AGAACCTGCGCGAGTTCGTGTTCAAGAACATCGACGGCTACTTCAAAATCTACAGCAAGCACACCCCT
ATCAACCTCGTGCGGGATCTGCCTCAGGGCTTCTCTGCTCTGGAACCCCTGGTGGATCTGCCCATCGG
CATCAACATCACCCGGTTTCAGACACTGCTGGCCCTGCACAGAAGCTACCTGACACCTGGCGATAGCA
GCAGCGGATGGACAGCTGGTGCCGCCGCTTACTACGTGGGATACCTCCAGCCTAGAACCTTCCTGCTG
AAGTACAACGAGAACGGCACCATCACCGACGCCGTGGATTGTGCTCTGGACCCTCTGAGCGAGACAAA
GTGCACCCTGAAGTCCTTCACCGTGGAAAAGGGCATCTACCAGACCAGCAACTTCCGGGTGCAGCCCA
CCGAATCCATCGTGCGGTTCCCCAATATCACCAATCTGTGCCCCTTCGGCGAGGTGTTCAATGCCACC
AGATTCGCCTCTGTGTACGCCTGGAACCGGAAGCGGATCAGCAATTGCGTGGCCGACTACTCCGTGCT
GTACAACTCCGCCAGCTTCAGCACCTTCAAGTGCTACGGCGTGTCCCCTACCAAGCTGAACGACCTGT
GCTTCACAAACGTGTACGCCGACAGCTTCGTGATCCGGGGAGATGAAGTGCGGCAGATTGCCCCTGGA
CAGACCGGCAATATCGCCGACTACAACTACAAGCTGCCCGACGACTTCACCGGCTGTGTGATTGCCTG
GAACAGCAACAACCTGGACTCCAAAGTCGGCGGCAACTACAATTACCTGTACCGGCTGTTCCGGAAGT
CCAATCTGAAGCCCTTCGAGCGGGACATCAGCACCGAAATCTATCAGGCCGGCAGCACCCCTTGCAAT
GGCGTGAAGGGCTTTAACTGCTACTTCCCACTGCAAAGCTACGGCTTCcqqCCAACATACGGCGTGGG
CTACCAGCCTTACAGAGTGGTGGTGCTGAGCTTCGAGCTGCTGCATGCTCCTGCCACAGTGTGCGGCC
CTAAGAAATCCACCAATCTCGTGAAGAACAAATGCGTCAACTTCAATTTCAACGGCCTGACCGGCACC
GGCGTGCTGACAGAGAGCAACAAGAAGTTCCTGCCATTCCAGCAGTTCGGCCGGGACATTGCCGATAC
CACAGATGCCGTCAGAGATCCCCAGACACTGGAAATCCTGGACATCACCCCATGCAGCTTCGGCGGAG
TGTCTGTGATCACCCCTGGCACCAACACCAGCAATCAGGTGGCAGTGCTGTACCAGGGCGTCAACTGT
ACAGAGGTGCCAGTGGCCATTCACGCCGATCAGCTGACCCCTACTTGGCGGGTGTACTCCACAGGCAG
CAATGTGTTTCAGACCAGAGCCGGCTGTCTGATCGGAGCCGAGCACGTGAACAATAGCTACGAGTGCG
ACATCCCCATCGGCGCTGGCATCTGCGCCTCTTACCAGACACAGACCAACAGCCACAGACGGGCTAGA
AGCGTGGCCAGCCAGAGCATCATTGCCTACACAATGTCTCTGGGCGCCGAGAACAGCGTGGCCTACTC
CAACAACTCTATCGCTATCCCCACCAATTTCACCATCAGCGTGACCACCGAGATCCTGCCTGTGTCCA
TGACCAAGACCAGCGTGGACTGCACCATGTACATCTGCGGCGATTCCACCGAGTGCTCCAACCTGCTG
CTCCAGTACGGCAGCTTCTGCACCCAGCTGAATAGAGCCCTGACAGGGATCGCCGTGGAACAGGACAA
GAACACCCAAGAGGTGTTCGCCCAAGTGAAGCAAATCTACAAGACCCCTCCTATCAAGGACTTCGGCG
GCTTCAACTTCAGCCAGATTCTGCCCGATCCTAGCAAGCCCAGCAAGCGGAGCTTCATCGAGGACCTG
CTGTTCAACAAAGTGACACTGGCCGACGCCGGCTTCATCAAGCAGTACGGCGATTGTCTGGGCGACAT
TGCAGCCAGGGATCTGATCTGCGCCCAGAAGTTTAACGGACTGACAGTGCTGCCTCCTCTGCTGACCG
ATGAGATGATCGCCCAGTACACATCTGCCCTGCTGGCCGGCACAATCACAAGCGGCTGGACATTTGGA
GCTGGCGCCGCTCTCCAGATTCCATTCGCTATGCAGATGGCCTACAGGTTCAACGGCATCGGAGTGAC
CCAGAATGTGCTGTACGAGAACCAGAAGCTGATCGCCAACCAGTTCAACAGCGCCATCGGCAAGATCC
AGGACAGCCTGAGCAGCACAGCAAGCGCCCTGGGAAAGCTCCAGGACGTGGTCAACCAGAATGCCCAG
GCACTGAACACCCTGGTCAAGCAGCTGTCCTCCAACTTCGGCGCCATCTCTAGCGTGCTGAACGATAT
CCTGAGCAGACTGGACCCACCTGAGGCCGAGGTGCAGATCGACAGACTGATCACAGGCCGGCTCCAGA
GCCTCCAGACATACGTTACACAGCAGCTGATCAGAGCCGCCGAGATTAGAGCCTCTGCCAATCTGGCC
GCCACCAAGATGTCTGAGTGTGTGCTGGGCCAGAGCAAGAGAGTGGACTTTTGCGGCAAGGGCTACCA
CCTGATGAGCTTCCCTCAGTCTGCACCACACGGCGTGGTGTTTCTGCACGTGACATACGTGCCCGCTC
AAGAGAAGAACTTTACCACCGCTCCAGCCATCTGCCACGACGGCAAAGCCCACTTTCCTAGAGAAGGC
GTGTTCGTGTCCAACGGCACCCATTGGTTCGTGACCCAGCGGAACTTCTACGAGCCCCAGATCATCAC
CACCGACAACACCTTCGTGTCCGGCAACTGCGACGTCGTGATCGGCATTGTGAACAATACCGTGTACG
ACCCTCTCCAGCCGGAACTGGACTCCTTCAAAGAGGAACTGGATAAGTACTTTAAGAACCACACAAGC
CCCGACGTGGACCTGGGCGATATCAGCGGAATCAATGCCAGCGTCGTGAACATCCAGAAAGAGATCGA
CCGGCTGAACGAGGTGGCCAAGAATCTGAACGAGAGCCTGATCGACCTGCAAGAACTGGGGAAGTACG
AGCAGTACATCAAGTGGCCTTGGTACATCTGGCTGGGCTTTATCGCCGGACTGATTGCCATCGTGATG
GTCACAATCATGCTGTGTTGCATGACCAGCTGTTGCAGCTGCCTGAAGGGCTGCTGTAGCTGTGGCTC
CTGCTGCTGAGCGGCCGCAGATCTGCTGTGCCTTCTAGTTGCCAGCCATCTGTTGTTTGCCCCTCCCC
CGTGCCTTCCTTGACCCTGGAAGGTGCCACTCCCACTGTCCTTTCCTAATAAAATGAGGAAATTGCAT
CGCATTGTCTGAGTAGGTGTCATTCTATTCTGGGGGGTGGGGTGGGGCAGGACAGCAAGGGGGAGGAT
TGGGAAGACAATAGCAGGCATGCTGGGGATGCGGTGGGCTCTATGGCTACCCAGGTGCTGAAGAATTG
ACCCGGTTCCTCCTGGGCCAGAAAGAAGCAGGCACATCCCCTTCTCTGTGACACACCCTGTCCACGCC
CCTGGTTCTTAGTTCCAGCCCCACTCATAGGACACTCATAGCTCAGGAGGGCTCCGCCTTCAATCCCA
CCCGCTAAAGTACTTGGAGCGGTCTCTCCCTCCCTCATCAGCCCACCAAACCAAACCTAGCCTCCAAG
AGTGGGAAGAAATTAAAGCAAGATAGGCTATTAAGTGCAGAGGGAGAGAAAATGCCTCCAACATGTGA
GGAAGTAATGAGAGAAATCATAGAATTTTAAGGCCATGATTTAAGGCCATCATGGCCTTAATCTTCCG
CTTCCTCGCTCACTGACTCGCTGCGCTCGGTCGTTCGGCTGCGGCGAGCGGTATCAGCTCACTCAAAG
GCGGTAATACGGTTATCCACAGAATCAGGGGATAACGCAGGAAAGAACATGTGAGCAAAAGGCCAGCA
AAAGGCCAGGAACCGTAAAAAGGCCGCGTTGCTGGCGTTTTTCCATAGGCTCCGCCCCCCTGACGAGC
ATCACAAAAATCGACGCTCAAGTCAGAGGTGGCGAAACCCGACAGGACTATAAAGATACCAGGCGTTT
CCCCCTGGAAGCTCCCTCGTGCGCTCTCCTGTTCCGACCCTGCCGCTTACCGGATACCTGTCCGCCTT
TCTCCCTTCGGGAAGCGTGGCGCTTTCTCATAGCTCACGCTGTAGGTATCTCAGTTCGGTGTAGGTCG
TTCGCTCCAAGCTGGGCTGTGTGCACGAACCCCCCGTTCAGCCCGACCGCTGCGCCTTATCCGGTAAC
TATCGTCTTGAGTCCAACCCGGTAAGACACGACTTATCGCCACTGGCAGCAGCCACTGGTAACAGGAT
TAGCAGAGCGAGGTATGTAGGCGGTGCTACAGAGTTCTTGAAGTGGTGGCCTAACTACGGCTACACTA
GAAGAACAGTATTTGGTATCTGCGCTCTGCTGAAGCCAGTTACCTTCGGAAAAAGAGTTGGTAGCTCT
TGATCCGGCAAACAAACCACCGCTGGTAGCGGTGGTTTTTTTGTTTGCAAGCAGCAGATTACGCGCAG
AAAAAAAGGATCTCAAGAAGATCCTTTGATCTTTTCTACGGGGTCTGACGCTCAGTGGAACGAAAACT
CACGTTAAGGGATTTTGGTCATGAGATTATCAAAAAGGATCTTCACCTAGATCCTTTTAAATTAAAAA
TGAAGTTTTAAATCAATCTAAAGTATATATGAGTAAACTTGGTCTGACAGTTACCAATGCTTAATCAG
TGAGGCACCTATCTCAGCGATCTGTCTATTTCGTTCATCCATAGTTGCCTGACTCGGGGGGGGGGGGC
GCTGAGGTCTGCCTCGTGAAGAAGGTGTTGCTGACTCATACCAGGCCTGAATCGCCCCATCATCCAGC
CAGAAAGTGAGGGAGCCACGGTTGATGAGAGCTTTGTTGTAGGTGGACCAGTTGGTGATTTTGAACTT
TTGCTTTGCCACGGAACGGTCTGCGTTGTCGGGAAGATGCGTGATCTGATCCTTCAACTCAGCAAAAG
TTCGATTTATTCAACAAAGCCGCCGTCCCGTCAAGTCAGCGTAATGCTCTGCCAGTGTTACAACCAAT
TAACCAATTCTGATTAGAAAAACTCATCGAGCATCAAATGAAACTGCAATTTATTCATATCAGGATTA
TCAATACCATATTTTTGAAAAAGCCGTTTCTGTAATGAAGGAGAAAACTCACCGAGGCAGTTCCATAG
GATGGCAAGATCCTGGTATCGGTCTGCGATTCCGACTCGTCCAACATCAATACAACCTATTAATTTCC
CCTCGTCAAAAATAAGGTTATCAAGTGAGAAATCACCATGAGTGACGACTGAATCCGGTGAGAATGGC
AAAAGCTTATGCATTTCTTTCCAGACTTGTTCAACAGGCCAGCCATTACGCTCGTCATCAAAATCACT
CGCATCAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACAATATTT
TCACCTGAATCAGGATATTCTTCTAATACCTGGAATGCTGTTTTCCCGGGGATCGCAGTGGTGAGTAA
CCATGCATCATCAGGAGTACGGATAAAATGCTTGATGGTCGGAAGAGGCATAAATTCCGTCAGCCAGT
TTAGTCTGACCATCTCATCTGTAACATCATTGGCAACGCTACCTTTGCCATGTTTCAGAAACAACTCT
GGCGCATCGGGCTTCCCATACAATCGATAGATTGTCGCACCTGATTGCCCGACATTATCGCGAGCCCA
TTTATACCCATATAAATCAGCATCCATGTTGGAATTTAATCGCGGCCTCGAGCAAGACGTTTCCCGTT
GAATATGGCTCATAACACCCCTTGTATTACTGTTTATGTAAGCAGACAGTTTTATTGTTCATGATGAT
ATATTTTTATCTTGTGCAATGTAACATCAGAGATTTTGAGACACAACGTGGCTTTCCCCCCCCCCCCA
TTATTGAAGCATTTATCAGGGTTATTGTCTCATGAGCGGATACATATTTGAATGTATTTAGAAAAATA
AACAAATAGGGGTTCCGCGCACATTTCCCCGAAAAGTGCCACCTGACGTCTAAGAAACCATTATTATC
ATGACATTAACCTATAAAAATAGGCGTATCACGAGGCCCTTTCGTC

### Cell Lines and Transfection

HEK293T cells were transfected using the polyethylenimide (PEI) method (Boussif, O. et al. A versatile vector for gene and oligonucleotide transfer into cells in culture and in vivo: polyethylenimine. Proc. Natl. Acad. Sci. 92, 7297 LP-7301, 1995). For PEI transfection, 6×10⁵ cells were seeded in 6-well plates one day before transfection. The cells were transfected with 2.5 μg plasmid (equimolar amounts, filled with empty vector) and 7.5 μg PEI in DMEM without any supplements. After 6 hour incubation, medium was exchanged to DMEM with 10% FCS and 1% Pen/Strep.

FIG. 61 shows Western blot analysis of HEK293T cell lysates 48 hours following transfection with pURVac T2_17 RBD. The antigen was detected using an anti-SARS-CoV-2 Spike antibody (upper panel). Tubulin levels were monitored using an anti-tubulin antibody as loading control (lower panel). Theoretical molecular weight in kilo Dalton (kDa) calculated from amino acid sequence.

FIG. 62A shows Western blot analysis of HEK293T cell lysates 48 hours following transfection with pURVac T2_29 DNA constructs (T2_29, T2_29+dER, T2_29+Q498R+dER). The respective antigens were detected using an anti-SARS-CoV-2 Spike antibody (upper panel). Tubulin levels were monitored using an anti-tubulin antibody as loading control (lower panel). Theoretical molecular weight in kilo Dalton (kDa) calculated from amino acid sequence.

FIG. 62B shows flow cytometry analysis of HEK293T cells 48 hours following transfection with pURVac DNA vaccines (T2_29, T2_29+Q498R, and T2_29+Q498R+dER) using serum obtained before (neg) and after infection with SARS-CoV-2 (ref+inf) as primary antibody for cell surface staining. Depicted is % positive cells and mean fluorescence intensity.

### Results

In order to test expression of the different T2_29 SARS COV-2 Spike DNA constructs, Western blot analysis was performed. Staining of the membrane with a polyclonal SARS-CoV-2 rabbit antibody showed that the pURVac constructs encoding the respective antigen were successfully expressed at the expected band of approximately of 35 kDa for the DNA vaccine constructs pURVac RBD T2_17 (FIG. 61) and around 180 kDa for pURVac T2_29+Q498R+dER (FIG. 62A). The band of the RBD T2_17 appears in the immunoblot blot slightly higher due to glycosylation compared to the calculated molecular weight in kDa. The controls showed no bands as expected (FIG. 61).

FIG. 62A shows that the DNA vaccine vector encoding the T2_29+Q498R+dER Spike antigen was successfully generated. The immunoblot stained with a polyclonal SARS-CoV-2 S specific rabbit antibody revealed good antigen expression and showed the expected band at around 180 kDa. The band of the SARS COV-2 T2_29+Q498R+dER appears in the immunoblot blot higher due to glycosylation compared to the calculated molecular weight in kDa based on the amino acid sequence. The cleavage spike product S1 subunit can be seen at around 110 kDa as the furin cleavage site in the analysed constructs is intact. When the cells were not infected, no expression could be detected as expected.

Flow cytometry was carried to determine the surface display of different T2_29 Spike DNA vaccine constructs. Results of using sera obtained before (neg) and after infection with SARS-CoV-2 (ref and pos) as primary antibodies are displayed in FIG. 62B. There was a relatively high background of positive cells in the negative serum and a low background signal when using only the secondary antibody as a control. Only minor differences in the percentage of positive cells between the T2_29 Spike constructs were observed. However, the mean fluorescence intensity (MFI) revealed higher values for Super S T2_29+Q498R compared to Super S T2_29 and even higher fluorescence signals after transfection with Super S T2_29+Q498R+ΔER. Taken together, the percentage of positive cells was comparable, but MFIs varied between the T2_29 Spike DNA vaccine constructs. Transfection of T2_29+Q498R+ΔER led to increased MFIs compared to T2_29+Q498R including the ER retention motif and T2_29 without modifications, indicating enhanced surface display of Spike protein with deleted ER-retention motif (ΔER).

### EXAMPLE 40

This example describes MVA vaccine constructs according to embodiments of the invention. In particular, the example describes the nucleic acid sequences of the MVA transfer vector (SEQ ID NO:96), and recombinant MVA constructs pMVA_T2_17+tPA and pMVA_T2_29+Q498R+dER. Also shown is successful infection of HEK293T cells with rMVA COV_S_T2_17+tPA and CoV_S_T2_29+Q498R+dER constructs, and subsequent expression of the encoded antigen sequences.

### Mva Transfer Vector

The nucleic acid sequence of the MVA transfer vector is shown below. The sequence is the MVA.CR19 sequence: GenBank accession number: KY633487, version number KY633487.1, release date 28.03.2017, https://www.ncbi.nlm.nih.gov/nuccore/KY633487.1.

Sequences homologous to “transfer vector” used for site specific recombination

5′ flank shown in underline format.

3′ flank coloured in bold and underline format.

TK insertion locus of MVA.CR19 ranging from 86,851 to 88,561

>MVA transfer vector
Nucleic acid sequence
(SEQ ID NO: 96)
CTGAATATGAAGGAGCAAAAGGTTGTAACATTTTATTACCGTGTGGGATATAAAAGTCCTTGATCCAT
TGATCTGGAAACGGGCATCTCCATTTAAGACTAGACGCCACGGGGTTTAAAATACTAATCATGACATT
TTGTAGAGCGTAATTACTTAGTAAATCCGCCGTACTAGGTTCATTTCCTCCTCGTTTGGATCTCACAT
CAGAAATTAAAATAATCTTAGAAGGATGCAGTTGTTTTTTGATGGATCGTAGATATTCCTCATCAACG
AACCGAGTCACTAGAGTCACATCACGCAATCCATTTAAAATAGGATCATGATGGCGGCCGTCAATTAG
CATCCATTTGATGATCACTCCTAAATTATAGAAATGATCTCTCAAATAACGTATATGTGTACCGGGAG
CAGATCCTATATACACTACGGTGGCACCATCTAATATACCGTGTCGCTGTAACTTACTAAGAAAAAAT
AATTCTCCTAGTAATAGTTTTAACTGTCCTTGATACGGCAGTTTTTTTGCGACCTCATTTGCACTTTC
TGGTTCGTAATCTAACTCATTATCAATTTCCTCAAAATACATAAACGGTTTATCTAACGACACAACAT
CCATTTTTAAGTATTATATTAAAATTTAATCAATGTTTATTTTTAGTTTTTTAGATAAAAAATATAAT
ATTATGAGCCGACGTAACACTTTCTACACACCGATTGATACATATCATTACCTCCTATTATCTCTATC
TCGGTTTCCTCACCCAATCGTTTAGAAAAGGAAGCCTCCTTAAAGCATTTCATACACACAGCAGTTAG
TTTTACCACCATTTCAGATAATGGAATAAGATTCAAAATATTATTAAACGGTTTACGTTGAAATGTCC
CATCGAGTGCGGCTACTATAACTATTTTTCCTTCGTTTGCCATACGCTCACAGAATTCAACAATGTCT
GGAAAGAACTGTCCTTCATCGATACCTATCACGGAGAAATCTGTAATTGATTCCAAGACATCACATAG
TTTAGTTGCTTCCAATGCTTCAAAATTATTCTTATCATGCGTCCATAGTCCCGTTCCGTATCTATTAT
CGTTAGAATATTTTATAGTCACGCATTTATATTGAGCTATTTGATAACGTCTAACTCGTCTAATTAAT
TCTGTACTTTTACCTGAAAACATGGGGCCGATTATCAACTGAATATGTCCGCCGTTCATGATGACAAT
AAAGAATTAATTATTGTTCACTTTATTCGACTTTAATATATCCATCACGTTAGAAAATGCGATATCGC
GACGAGGATCTATGTATCTAATAGGATCTATTGCGGTGGTAGCTAGAGAGGATTCTTTTTTGAATCGC
ATCAAACTAATCACAAAGTCGAACAAATATCCTTTATTAAGTTTGACCCTTCCATCTGTAACAATAGG
GACCTTGTTAAACAGTTTTTTAAAATCTTGAAAGTCTGTGAATTTTGTCAATTGTCTGTATTCCTCTG
AAAGAGATTCATAACAATGACCCACGGCTTCTAATTTATTTTTTGATTGGATCAATAATAATAACAGA
AAGTCTAGATATTGAGTGATTTGCAATATATCAGATAATGAAGATTCATCATCTTGACTAGCCAAATA
CTTAAAAAATGAATCATCATCTGCGAAGAACATCGTTAAGAGATACTGGTTGTGATCCATTTATTGAT
CGCAAAAGCTT

As a transfer vector, the pMVA Trans TK under a mH5 poxviral promoter was used for the generation of recombinant MVAs, described below.

### T2_17+tPA

The promoter sequence is shown in underline format.

The terminator is shaded grey.

The gene of interest, namely T2_17+tPA (including start and stop codon), is shown in bold and underline format.

>pMVA Trans TK mH5 T2_17+tPA
Nucleic acid sequence
(SEQ ID NO: 97)
AAAAAATGAAAATAAATACAAAGGTTCTTGAGGGTTGTGTTAAATTGAAAGCGAGAAATAATCATAAA
TTGGTACCGCCACCATGGATGCTATGAAGAGGGGCCTGTGCTGCGTGCTGCTTCTGTGTGGCGCTGTG
TTTGTGTCTCCTAGCGCCGCTAGAGTGGCCCCTACCAAAGAAGTCGTGCGGTTCCCCAACATCACCAA
TCTGTGCCCTTTCGGCGAGGTGTTCAACGCCACCAAGTTTCCCTCTGTGTACGCCTGGGAGCGCAAAA
AGATCAGCAACTGCGTGGCCGACTACAGCGTGCTGTACAACAGCACCAGCTTCAGCACCTTCAAGTGC
TACGGCGTGTCACCCACCAAGCTGAACGACCTGTGCTTCACCAACGTGTACGCCGACAGCTTCGTGAT
CAGAGGCGACGAAGTGCGGCAGATTGCCCCTGGACAAACAGGCGTGATCGCCGATTACAACTACAAGC
TGCCCGACGACTTCACCGGCTGTGTGATCGCCTGGAACACCAACAACATCGACAGCACCACCGGCGGC
AACTACAACTACCTGTACAGAAGCCTGCGGAAGTCTAAGCTGAAGCCCTTCGAGCGGGACATCAGCAG
CGACATCTATAGCCCTGGCGGCAAGCCTTGTTCTGGCGTGGAAGGCTTCAACTGCTACTACCCTCTGC
GGAGCTACGGCTTCTTCCCCACAAATGGCACAGGCTACCAGCCTTACAGAGTGGTGGTCCTGAGCTTC

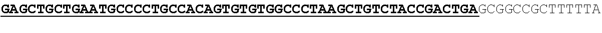

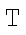

### T2_29+Q498R+dER

The gene of interest, namely T2_29+Q498R+dER (including start and stop codon), is shown in bold and underline.

Again, the promoter sequence is shown in underline format.

The terminator is shaded grey.

>pMVA Trans TK mH5 T2_29 + Q498R + dER
Nucleic acid sequence
(SEQ ID NO: 98)
AAAAAATGAAAATAAATACAAAGGTTCTTGAGGGTTGTGTTAAATTGAAAGCGAGAAATAATCATAAATTGGT
ACCGCCACCATGTTCGTGTTTCTGGTGCTGCTGCCTCTGGTGTCCAGCCAGTGCGTGAACTTCACCAACAGAA
CCCAGCTGCCTAGCGCCTACACCAACAGCTTTACCAGAGGCGTGTACTACCCCGACAAGGTGTTCAGATCCA
GCGTGCTGCACTCTACCCAGGACCTGTTCCTGCCTTTCTTCAGCAACGTGACCTGGTTCCACGCCATCAGCGG
CACCAATGGCACCAAGAGATTCGACAACCCCGTGCTGCCCTTCAACGACGGGGTGTACTTTGCCAGCACCGA
GAAGTCCAACATCATCAGAGGCTGGATCTTCGGCACCACACTGGACAGCAAGACCCAGAGCCTGCTGATCG
TGAACAACGCCACCAACGTGGTCATCAAAGTGTGCGAGTTCCAGTTCTGCAACGACCCCTTCCTGGGCGTGT
ACCACAAGAACAACAAGAGCTGGATGGAAAGCGAGTTCCGGGTGTACAGCAGCGCCAACAACTGCACCTT
CGAGTACGTGTCCCAGCCTTTCCTGATGGACCTGGAAGGCAAGCAGGGCAACTTCAAGAACCTGCGCGAGT
TCGTGTTCAAGAACATCGACGGCTACTTCAAAATCTACAGCAAGCACACCCCTATCAACCTCGTGCGGGATC
TGCCTCAGGGCTTCTCTGCTCTGGAACCCCTGGTGGATCTGCCCATCGGCATCAACATCACCCGGTTTCAGAC
ACTGCTGGCCCTGCACAGAAGCTACCTGACACCTGGCGATAGCAGCAGCGGATGGACAGCTGGTGCCGCCG
CTTACTACGTGGGATACCTCCAGCCTAGAACCTTCCTGCTGAAGTACAACGAGAACGGCACCATCACCGACG
CCGTGGATTGTGCTCTGGACCCTCTGAGCGAGACAAAGTGCACCCTGAAGTCCTTCACCGTGGAAAAGGGC
ATCTACCAGACCAGCAACTTCCGGGTGCAGCCCACCGAATCCATCGTGCGGTTCCCCAATATCACCAATCTGT
GCCCCTTCGGCGAGGTGTTCAATGCCACCAGATTCGCCTCTGTGTACGCCTGGAACCGGAAGCGGATCAGCA
ATTGCGTGGCCGACTACTCCGTGCTGTACAACTCCGCCAGCTTCAGCACCTTCAAGTGCTACGGCGTGTCCCC
TACCAAGCTGAACGACCTGTGCTTCACAAACGTGTACGCCGACAGCTTCGTGATCCGGGGAGATGAAGTGC
GGCAGATTGCCCCTGGACAGACCGGCAATATCGCCGACTACAACTACAAGCTGCCCGACGACTTCACCGGCT
GTGTGATTGCCTGGAACAGCAACAACCTGGACTCCAAAGTCGGCGGCAACTACAATTACCTGTACCGGCTGT
TCCGGAAGTCCAATCTGAAGCCCTTCGAGCGGGACATCAGCACCGAAATCTATCAGGCCGGCAGCACCCCTT
GCAATGGCGTGAAGGGCTTTAACTGCTACTTCCCACTGCAAAGCTACGGCTTCcggCCAACATACGGCGTGG
GCTACCAGCCTTACAGAGTGGTGGTGCTGAGCTTCGAGCTGCTGCATGCTCCTGCCACAGTGTGCGGCCCTA
AGAAATCCACCAATCTCGTGAAGAACAAATGCGTCAACTTCAATTTCAACGGCCTGACCGGCACCGGCGTGC
TGACAGAGAGCAACAAGAAGTTCCTGCCATTCCAGCAGTTCGGCCGGGACATTGCCGATACCACAGATGCC
GTCAGAGATCCCCAGACACTGGAAATCCTGGACATCACCCCATGCAGCTTCGGCGGAGTGTCTGTGATCACC
CCTGGCACCAACACCAGCAATCAGGTGGCAGTGCTGTACCAGGGCGTCAACTGTACAGAGGTGCCAGTGGC
CATTCACGCCGATCAGCTGACCCCTACTTGGCGGGTGTACTCCACAGGCAGCAATGTGTTTCAGACCAGAGC
CGGCTGTCTGATCGGAGCCGAGCACGTGAACAATAGCTACGAGTGCGACATCCCCATCGGCGCTGGCATCT
GCGCCTCTTACCAGACACAGACCAACAGCCACAGACGGGCTAGAAGCGTGGCCAGCCAGAGCATCATTGCC
TACACAATGTCTCTGGGCGCCGAGAACAGCGTGGCCTACTCCAACAACTCTATCGCTATCCCCACCAATTTCA
CCATCAGCGTGACCACCGAGATCCTGCCTGTGTCCATGACCAAGACCAGCGTGGACTGCACCATGTACATCT
GCGGCGATTCCACCGAGTGCTCCAACCTGCTGCTCCAGTACGGCAGCTTCTGCACCCAGCTGAATAGAGCCC
TGACAGGGATCGCCGTGGAACAGGACAAGAACACCCAAGAGGTGTTCGCCCAAGTGAAGCAAATCTACAA
GACCCCTCCTATCAAGGACTTCGGCGGCTTCAACTTCAGCCAGATTCTGCCCGATCCTAGCAAGCCCAGCAA
GCGGAGCTTCATCGAGGACCTGCTGTTCAACAAAGTGACACTGGCCGACGCCGGCTTCATCAAGCAGTACG
GCGATTGTCTGGGCGACATTGCAGCCAGGGATCTGATCTGCGCCCAGAAGTTTAACGGACTGACAGTGCTG
CCTCCTCTGCTGACCGATGAGATGATCGCCCAGTACACATCTGCCCTGCTGGCCGGCACAATCACAAGCGGC
TGGACATTTGGAGCTGGCGCCGCTCTCCAGATTCCATTCGCTATGCAGATGGCCTACAGGTTCAACGGCATC
GGAGTGACCCAGAATGTGCTGTACGAGAACCAGAAGCTGATCGCCAACCAGTTCAACAGCGCCATCGGCAA
GATCCAGGACAGCCTGAGCAGCACAGCAAGCGCCCTGGGAAAGCTCCAGGACGTGGTCAACCAGAATGCC
CAGGCACTGAACACCCTGGTCAAGCAGCTGTCCTCCAACTTCGGCGCCATCTCTAGCGTGCTGAACGATATC
CTGAGCAGACTGGACCCACCTGAGGCCGAGGTGCAGATCGACAGACTGATCACAGGCCGGCTCCAGAGCCT
CCAGACATACGTTACACAGCAGCTGATCAGAGCCGCCGAGATTAGAGCCTCTGCCAATCTGGCCGCCACCAA
GATGTCTGAGTGTGTGCTGGGCCAGAGCAAGAGAGTGGACTTTTGCGGCAAGGGCTACCACCTGATGAGCT
TCCCTCAGTCTGCACCACACGGCGTGGTGTTTCTGCACGTGACATACGTGCCCGCTCAAGAGAAGAACTTTA
CCACCGCTCCAGCCATCTGCCACGACGGCAAAGCCCACTTTCCTAGAGAAGGCGTGTTCGTGTCCAACGGCA
CCCATTGGTTCGTGACCCAGCGGAACTTCTACGAGCCCCAGATCATCACCACCGACAACACCTTCGTGTCCGG
CAACTGCGACGTCGTGATCGGCATTGTGAACAATACCGTGTACGACCCTCTCCAGCCGGAACTGGACTCCTT
CAAAGAGGAACTGGATAAGTACTTTAAGAACCACACAAGCCCCGACGTGGACCTGGGCGATATCAGCGGA
ATCAATGCCAGCGTCGTGAACATCCAGAAAGAGATCGACCGGCTGAACGAGGTGGCCAAGAATCTGAACG
AGAGCCTGATCGACCTGCAAGAACTGGGGAAGTACGAGCAGTACATCAAGTGGCCTTGGTACATCTGGCTG
GGCTTTATCGCCGGACTGATTGCCATCGTGATGGTCACAATCATGCTGTGTTGCATGACCAGCTGTTGCAGC

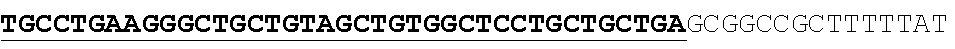

### Methods

### Antigen Design

The antigens were synthesized at Geneart/Thermo Fisher (Regensburg, Germany). The GeneOptimizer algorithm was used to minimize sequence homology and adapt the sequences to human codon usage (Raab, D., Graf, M., Notka, F., Schödl, T. & Wagner, R. The GeneOptimizer Algorithm: using a sliding window approach to cope with the vast sequence space in multiparameter DNA sequence optimization. Syst. Synth. Biol. 4, 215-225, 2010). All constructs were cloned using standard molecular biology methods. Mutations in the T2_29 antigen were introduced by PCR or NEBuilder HIFI DNA Assembly Kit (New England Biolabs, Ipswich, USA) according to manufacturer's instructions. The modifications included the deletion of the endoplasmatic reticulum signal (dER) in order to increase surface expression. Another modification to the T2_29 antigen was the Q498R amino acid substitution The Q498R amino acid substitution was identified using a high-throughput yeast surface display in vitro evolution technique (Zahradnik, J., Marciano, S., Shemesh, M., Zoler, E., Harari, D., & Chiaravalli, J. et al. SARS-CoV-2 variant prediction and antiviral drug design are enabled by RBD in vitro evolution. Nature Microbiology, 6 (9), 1188-1198, 2021). This mutation was described as a potentially new evolving mutation with higher infectivity in combination with the N501Y mutation. It was shown that in combination with N501Y mutation which was previously detected in α, β, and γ SARS COV-2 variants of concern, the affinity of the RBD binding to the ACE2 increased four-fold compared to N501Y alone. Moreover, the mutation Q498R in combination with N501Y also appeared in the Omicron variant of concern. Therefore, Q498R mutation is a significant potential mutation that can increase the infectivity of future SARS-CoV-2 variants. It was shown by this in silico evolution approach that the Q498R mutation led to the formation of additional hydrogen bonds with ACE2 42N and 38D amino acids. These additional hydrogen bonds may contribute to the increased affinity to ACE2 (Xue, T., Wu, W., Guo, N., Wu, C., Huang, J., & Lai, L. et al. Single point mutations can potentially enhance infectivity of SARS-CoV-2 revealed by in silico affinity maturation and SPR assay. RSC Advances, 11(24), 14737-14745, 2021).

All constructs were cloned into the different plasmid backbones using Kpnl-HF and Notl-HF (New England Biolabs, Ipswich, USA). The sequence of the plasmids was verified by Sanger sequencing. Plasmids were prepared, depending on amount, with alkaline lysis or commercially available kits according to manufacturer's instructions (Plasmid Midi plus, EndoFree Plasmid Mega Kit, Qiagen, Hilden, Germany).

For initial biochemical and immunological characterization, the constructs were cloned into pURVac, a derivative of a DNA vaccine vector with a proven track record in various NHP and clinical trials (Asbach, B. et al. Priming with a Potent HIV-1 DNA Vaccine Frames the Quality of Immune Responses prior to a Poxvirus and Protein Boost. J. Virol. 93, 2019; Sarwar, U. N. et al. Safety and immunogenicity of DNA vaccines encoding Ebolavirus and Marburgvirus wild-type glycoproteins in a phase I clinical trial. J. Infect. Dis. 211, 549-557, 2015; Joseph, S. et al. A Comparative Phase I Study of Combination, Homologous Subtype-C DNA, MVA, and Env gp140 Protein/Adjuvant HIV Vaccines in Two Immunization Regimes. Front. Immunol. 8, 149, 2017; Pantaleo, G. et al. Safety and immunogenicity of a multivalent HIV vaccine comprising envelope protein with either DNA or NYVAC vectors (HVTN 096): a phase 1b, double-blind, placebo-controlled trial. Lancet HIV 6, e737-e749, 2019) where antigens are under the control of a human cytomegalovirus (CMV) promoter in combination with a human T-cell leukemia virus-1 (HTLV-1) regulatory element (Barouch, D. H. et al. A human T-cell leukemia virus type 1 regulatory element enhances the immunogenicity of human immunodeficiency virus type 1 DNA vaccines in mice and nonhuman primates. J. Virol. 79, 8828-8834, 2005) and a bovine growth hormone poly-A terminator.

### Cell Lines and Viral Infection

HEK293T cells maintained and grown in Dulbecco's MEM (DMEM) supplemented with 10% fetal calf serum (FCS) and 1% Pen/Strep (PS) at 5% CO2 and 37° C. in a humidified incubator.

For expression analysis of recombinant MVAs 6×105 HEK293T cells were seeded 24 h before infection. For infection HEK293T cells were infected with each individual recombinant MVA vectors at MOI 2.0 and harvested after 24 h.

### Design of MVA Transfer Vectors

For the generation of recombinant MVA expressing SARS-CoV-2 RBD T2_17+tPA and SARS COV-2 T2_29+Q498R+dER the shuttle vectors pMVA Trans TK-SARS-CoV-2 RBD T2_17 and pMVA Trans TK SARS COV-2 T2_29+Q498R+dER were cloned using standard molecular biology techniques. The MVA shuttle vectors were designed in a way that the genes of interest (FIG. 1) can be inserted into the thymidine kinase (TK) locus J2R of the parental virus MVA CR19 TK-GFP under the transcriptional control of the early/late modified H5 promoter (mH5) via homologous recombination. The MVA shuttle vectors also include the reporter gene β-galactosidase (β-Gal) between the two left arm sequences of the TK locus for screening of recombinant MVAs. After several plaque purification rounds the reporter gene gets lost after an internal homologous recombination event resulting in a pure recombinant MVA.

### Generation of Recombinant MVA Vectors

MVA is adapted to replication in avian cells. For production of MVA a host is therefore preferred such as primary chicken embryo fibroblasts (CEF) or AGE1.CR.pIX that is derived from duck retina cells. As opposed to primary cells, an immortalized (or continuous) cell line such as AGE1.CR.pIX has several advantages: the cell substrate can be retrieved out of locally stored cryocultures and thus is resilient to supply constraints. An immortal cell line can furthermore be characterized against adventitious agents at the level of the cell bank, well ahead of the actual production processes. The AGE1.CR.pIX cell line (as opposed to primary material) furthermore proliferates in suspension in media free of animal derived components. This property allows highly efficient and scalable fed-batch production processes for poxviruses of different genera (Jordan I, Northoff S, Thiele M, Hartmann S, Horn D, Howing K, Bernhardt H, Oehmke S, von Horsten H, Rebeski D, Hinrichsen L, Zelnik V, Mueller W, & Sandig V, 2011. A chemically defined production process for highly attenuated poxviruses. Biologicals 39, 50-58. https://doi.org/10.1016/j.biologicals.2010.11.005. PMID 21237672).

Vaccinia viruses mature into infectious particles with one or three membranes without the requirement for budding. One consequence of this complex infectious cycle is that the majority of the wild-type virions remain associated with the producer cell and that only a small fraction of infectious activity can be measured in the culture supernatant. The development of the novel strain MVA-CR19 has been induced by this observation (Jordan I, Horn D, John K, & Sandig V, 2013. A genotype of modified vaccinia Ankara (MVA) that facilitates replication in suspension cultures in chemically defined medium. Viruses 5, 321-339. https://doi.org/10.3390/v5010321. PMID 23337383). MVA-CR19 is a strain of MVA with a unique genotype (Jordan I, Horn D, Thiele K, Haag L, Fiddeke K, & Sandig V, 2019. A Deleted Deletion Site in a New Vector Strain and Exceptional Genomic Stability of Plaque-Purified Modified Vaccinia Ankara (MVA). Virol Sin. https://doi.org/10.1007/s12250-019-00176-3. PMID 31833037). Point mutations in structural genes and recombination of a large portion of the inverted terminal repeat (ITR) at the left side of the linear genomic DNA have profound effects on the phenotype of MVA-CR19. For example, compared to wild-type, MVA-CR19 releases a larger number of infectious particles into the culture supernatant and replicates to higher infectious titers. Viral factors that impact immune responses of the host and the infectious cycle are encoded in the ITRs. The recombination event in MVA-CR 19 has changed the expression pattern of these factors (some were deleted, for others the gene dosehas been duplicated) with positive effects on efficacy and stability as a vaccine vector.

The potentially enhanced release of MVA-CR19 from host cells can also be seen in the CPE in adherent cells: whereas wild-type MVA tends to induce cell fusion and syncytia with well circumscribed plaques, infection with MVA-CR19 leads to a pattern consisting of large but loosely packed (unfused) plaques surrounded by isolated infected cells scattered at greater distances to the primary plaque or localized in comets.

The generation and isolation of recombinant MVA is complex due to the large size of the viral genome (178 kb). The most commonly used technique relies on homologous recombination in infected host cells with a shuttle plasmid that contains the gene of interest. The recombinant viruses must be isolated and purified from a vast background of contaminating parental viruses without the insert. While MVA-CR 19 has advantages for production and vaccine efficacy it can be more complex to purify due to the less confined nature of replication. Furthermore, for both wild-type and MVA-CR19, selection against expression and maintenance of a transgene may occur if the novel sequence impairs the infectious cycle.

For generation of recombinant MVAs the AGE1.CR.pIX cell line and MVA-CR19 were used. Adherent AGE1.CR.pIX cells were maintained in DMEM-F12 medium supplemented with 5% bovine serum (γ-irradiated, Sigma Aldrich/Merck, 12003C) and 2 mM GlutaMAX I (Gibco, 10565-018)).

For in vivo recombination adherent AGE1.CR.pIX (1×106 cells) were infected with parental MVA-CR19 TK-GFP with different MOls ranging from 0.5 to 0.006 plaque formining units (PFU). After 2 h the cells were transfected with 0.4 μg of the shuttle vector pMVA Trans-TK-SARS-CoV-2 RBD T2_17+tPA or pMVA Trans-TK SARS COV-2 T2_29+Q498R+dER using Effectene (Qiagen, Hilden, Germany) according to the manufacturer's instructions. After 48 h the cells were harvested, lysed by three times freeze-cycles, sonicated and used for agarose plaque purification rounds in order to obtain pure recombinant MVAs.

Recombinant MVAs expressing the SARS COV-2 RBD variants that were correctly inserted in the TK-locus and encode the β-galactosidase reporter gene were selected for further five plaque purification rounds after staining the cells with X-Gal (5-bromo-4-chloro-3-indolyl-β-D-galactopyranoside) until no remaining parental MVA-CR19 TK-GFP virus was detected by PCR screening. Three additional plaque purification rounds were performed until the transiently co expressing β-galactosidase reporter gene between two homologous left arm regions of the TK locus was deleted via an internal homologous recombination event and pure recombinant MVA was obtained. The recombinant MVAs were plaque purified for another three rounds in order to confirm no remaining reporter gene was detectable. The resulting recombinant MVA virus stock was grown on AGE1.CR.pIX cells, purified via two ultracentrifugation rounds over a 35% sucrose cushion and titrated. The sequence of the rMVA and absence of non-recombinant MVA was confirmed using PCR amplification, followed by Sanger Sequencing. The expression of rMVA was confirmed using HEK293T cells. Therefore, HEK293T cells were infected with a MOI of 2, harvested after 24 h and subjected to Western blot analysis.

### Western Blot Analysis

For expression analysis by Western blot analysis HEK293T cells were lysed in TDLB buffer (50 mM Tris, pH 8.0, 150 mM NaCl, 0.1% SDS, 1% Nonident P-40, 0.5% sodium deoxycholate) supplemented with protease inhibitors (Complete Mini, Roche, Basel, Swiss). Total protein concentration of the supernatants was determined by Bradford assay (Protein Assay, BioRad, Feldkirchen, Germany). The proteins were separated on SDS-PAGE under reducing conditions and blotted on a nitrocellulose membrane. Targets were probed with primary and secondary antibodies as listed below. HRP-labeled secondary antibodies and enhanced chemiluminescence substrate or Femto ECL (Thermo Fisher, Waltham, USA) were used for detection in a Chemilux Pro device (Intas, Göttingen, Germany). For loading control, the membrane was reprobed with an antibody against tubulin.

### Antibodies

The following antibodies were used: anti-SARS-CoV-2 Spike (1:1000, Sino Biological, Beijing, China, 40589-T62), anti-tubulin (DM1a, 1:1000, Santa Cruz Biotechnology, Heidelberg, Germany), goat anti-mouse-HRP (115-036-003, 1:5000, Jackson, West Grove, USA) and goat anti-rabbit-HRP (P0448, 1:2000, Dako, Santa Clara, USA).

FIG. 63A shows a schematic representation of the MVA genome and design of the recombinant SARS COV-2 RBD T2_17+tPA and SARS COV-2 Spike T2_29+Q498R+dER MVAs. The MVA genome consists of the left terminal region, the central conserved region and right conserved region and includes major deletion sites. The J2R region or TK locus was used to insert the gene of interest via homologous recombination between MVA DNA sequences (TK-L and TK-R) and the shuttle vector pMVA Trans mH5 TK SARS COV-2 RBD T2_17_tPA and SARS COV-2 Spike T2_29+Q498R+dER, respectively. Antigen expression is controlled by the strong early/late poxviral promoter mH5.

FIG. 63B shows expression analysis of T2_17+tPA RBD rMVA. Western blot analysis of HEK293T cell lysates 24 h following infection with rMVA encoding T2_17_tPA RBD antigen at an MOI of 2. As control cells were infected with empty rMVA CR19. The antigen was detected using an anti-SARS-CoV-2 Spike antibody (upper panel). Tubulin levels were monitored using an anti-tubulin antibody as loading control (lower panel). Theoretical molecular weight in kilo Dalton (kDa) calculated from amino acid sequence.

FIG. 64 shows expression analysis of T2_29+Q498R+dER rMVA. Western blot analysis of HEK293T cell lysates 24 h following infection with rMVA encoding T2_29+Q498R+dER antigen at an MOI of 2. As control cells were infected with empty rMVA CR19. As a positive control a cell lysate was prepared from cells transfected with pURVac SARS COV-2 Spike. The antigen was detected using an anti-SARS-CoV-2 Spike antibody (upper panel). Tubulin levels were monitored using an anti-tubulin antibody as loading control (lower panel). Theoretical molecular weight in kilo Dalton (kDa) calculated from amino acid sequence.

### Results

Since MVA as a recombinant viral vector is known to effectively boost DNA-primed specific immune responses against multiple infectious diseases (Asbach B, Kibler KV, Köstler J, et al. Priming with a Potent HIV-1 DNA Vaccine Frames the Quality of Immune Responses prior to a Poxvirus and Protein Boost. Journal of Virology. 2019 February; 93(3):e01529-18. DOI: 10.1128/jvi.01529-18. PMID: 30429343; PMCID: PMC6340047; Patricia Pérez, Miguel A. Martin-Acebes, Teresa Poderoso, Adrián Lázaro-Frias, Juan−Carlos Saiz, Carlos Óscar S. Sorzano, Mariano Esteban & Juan Garcia-Arriaza (2021) The combined vaccination protocol of DNA/MVA expressing Zika virus structural proteins as efficient inducer of T and B cell immune responses, Emerging Microbes & Infections, 10:1, 1441-1456, DOI: 10.1080/22221751.2021.1951624) recombinant MVAs were generated encoding the SARS-CoV-2 RBD T2_17 and SARS COV-2 T2_29 Q498R dER Spike antigens, respectively.

For in vitro characterisation of the rMVA vaccine constructs total cell lysates from HEK293T cells were prepared 24 h post infection, followed by Western blot analysis. Staining of the membrane with a polyclonal SARS-CoV-2 rabbit antibody showed that the rMVA constructs encoding the respective antigen were successfully expressed at the expected band of approximately of 35 kDa for the rMVA RBD T2_17+tPA (FIG. 63B) and around 180 kDa for the rMVA T2_29+Q498R+dER (FIG. 64). The band of the RBD T2_17+tPA appears in the immunoblot blot slightly higher due to glycosylation compared to the calculated molecular weigth in kDa. The controls showed no bands as expected.

FIG. 64 showed that the rMVA encoding the T2_29+Q498R+dER Spike antigen was successfully generated. The immunoblot stained with a polyclonal SARS-CoV-2 S specific rabbit antibody revealed good antigen expression of the recombinant MVAs and showed the expected band at around 180 kDa. The band of the SARS COV-2 T2_29+Q498R+dER appears in the immunoblot blot higher due to glycosylation compared to the calculated molecular weight in kDa based on the amino acid sequence. The cleavage spike product S1 subunit could be seen at around 110 kDa as the furin cleavage site in the analysed constructs is intact. When the cells were not infected, no expression could be detected as expected.

1. An isolated polypeptide, which comprises:
a) an amino acid sequence of SEQ ID NO:88 (COV_S_T2_29+Q498R+dER), or an amino acid sequence which has at least 98%, or 99% amino acid identity over its entire length with the amino acid sequence of SEQ ID NO:88 (COV_S_T2_29+Q498R+dER), optionally which comprises at least one, or all of the amino acid residues or deletions, at positions corresponding to the amino acid residue positions of SEQ ID NO:52, as shown in Table 9.11 below:
TABLE 9.11

| SARS2 S protein | COV_S_T2_29 + Q498R + dER |
|---|---|
| residue position | amino acid residue (SEQ ID |
| (SEQ ID NO: 52) | NO: 88) |
| 18 | F |
| 20 | N |
| 26 | S |
| 69 | — (deletion) |
| 70 | — (deletion) |
| 144 | — (deletion) |
| 417 | N |
| 484 | K |
| 498 | R |
| 501 | Y |
| 614 | G |
| 681 | H |
| 1255-1273 | — (deletion) |

or
b) an amino acid sequence of SEQ ID NO:87 (COV_S_T2_29+Q498R), or an amino acid sequence which has at least 99% amino acid identity over its entire length with the amino acid sequence of SEQ ID NO:87 (COV_S_T2_29+Q498R), optionally which comprises at least one, or all of the amino acid residues or deletions, at positions corresponding to the amino acid residue positions of SEQ ID NO:52, as shown in Table 9.8 below:
TABLE 9.8

| SARS2 S | |
|---|---|
| protein | |
| residue | |
| position | COV_S_T2_29 + Q498R |
| (SEQ ID | amino acid residue |
| NO: 52) | (SEQ ID NO: 87) |
| 18 | F |
| 20 | N |
| 26 | S |
| 69 | — (deletion) |
| 70 | — (deletion) |
| 144 | — (deletion) |
| 417 | N |
| 484 | K |
| 498 | R |
| 501 | Y |
| 614 | G |
| 681 | H |

2.-5. (canceled)

6. A polypeptide according to claim 1, which comprises an R amino acid residue at a position corresponding to amino acid residue position 498 of SEQ ID NO:52.

7. A polypeptide according to claim 1, which comprises a deletion of amino acid residues at positions corresponding to amino acid residue positions 1255-1273 of SEQ ID NO:52.

8. A polypeptide according to claim 1, which comprises an amino acid residue P at a position corresponding to amino acid residue position 986 of SEQ ID NO:52, and an amino acid residue P at a position corresponding to amino acid residue position 987 of SEQ ID NO:52.

9.-28. (canceled)

29. An isolated nucleic acid molecule encoding a polypeptide according to claim 1, or the complement thereof.

30.-31. (canceled)

32. A nucleic acid molecule according to claim 29, which encodes a polypeptide comprising an amino acid sequence of SEQ ID NO:87, wherein the nucleic acid molecule comprises a nucleotide sequence of SEQ ID NO:90, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with a nucleotide sequence of SEQ ID NO:90 over its entire length, or the complement thereof, or a polypeptide comprising an amino acid sequence of SEQ ID NO:88, wherein the nucleic acid molecule comprises a nucleotide sequence of SEQ ID NO:91, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with a nucleotide sequence of SEQ ID NO:91 over its entire length, or the complement thereof.

33.-35. (canceled)

36. A vector comprising a nucleic acid molecule of claim 29, optionally which further comprises a promoter operably linked to the nucleic acid, preferably wherein the promoter is for expression of a polypeptide encoded by the nucleic acid in mammalian cells.

37.-43. (canceled)

44. A vector according to claim 36, which is a vaccine vector, preferably which is a viral vaccine vector, a bacterial vaccine vector, an RNA vaccine vector, a DNA vaccine vector, or an mRNA vaccine vector.

45.-47. (canceled)

48. A vector according to claim 44, which is a pURVac vector, preferably which comprises a nucleic acid molecule encoding a polypeptide according to claim 1, wherein the vector comprises a nucleotide sequence of SEQ ID NO:95.

49. (canceled)

50. A vector according to claim 44, which is a Modified Vaccinia virus Ankara (MVA) vector, preferably wherein the MVA vector comprises a nucleic acid molecule encoding a polypeptide according to claim 1, wherein the vector comprises a nucleotide sequence of SEQ ID NO:98.

51.-55. (canceled)

56. A pharmaceutical composition comprising:
a) a polypeptide according to claim 1;
b) a nucleic acid molecule comprising a nucleotide sequence encoding the polypeptide of (a), or the complement thereof; or
c) a vector comprising a nucleic acid molecule of (b), and
a pharmaceutically acceptable carrier, excipient, or diluent, optionally which further comprises an adjuvant for enhancing an immune response in a subject to the polypeptide, or to a polypeptide encoded by the nucleic acid molecule, of the composition.

57.-76. (canceled)

77. A nucleic acid molecule according to claim 29, a vector comprising a nucleic acid molecule according to claim 29, or a pharmaceutical composition comprising a nucleic acid molecule according to claim 29, wherein the nucleic acid molecule comprises one or more modified nucleosides.

78.-79. (canceled)

80. A nucleic acid molecule, vector, or pharmaceutical composition according to claim 77, wherein the nucleic acid molecule comprises a messenger RNA (mRNA).

81. A nucleic acid molecule, a vector, or a pharmaceutical composition, according to claim 77, wherein the one or more modified nucleosides comprise a 1-methylpseudouridine modification, optionally, wherein at least 80% of the uridines in the open reading frame have been modified.

82.-83. (canceled)

84. A method of inducing an immune response to a coronavirus in a subject, or of immunizing a subject against a coronavirus, which comprises administering to the subject an effective amount of:
a) a polypeptide according to claim 1;
b) a nucleic acid molecule comprising a nucleotide sequence encoding the polypeptide of (a), or the complement thereof;
c) a vector comprising the nucleic acid molecule of (b);
d) a pharmaceutical composition comprising the polypeptide of (a) and a pharmaceutically acceptable carrier, excipient, or diluent; or
e) a pharmaceutical composition comprising the nucleic acid molecule of (b), or the vector of (c), and a pharmaceutically acceptable carrier, excipient, or diluent.

85. (canceled)

86. A method according to claim 84, which comprises administering a nucleic acid a vector, or a pharmaceutical composition, wherein the nucleic acid, vector, or pharmaceutical composition is administered as part of a heterologous prime boost regimen.

87. A method according to claim 86, wherein the heterologous prime boost regimen comprises a DNA prime followed by an MVA boost.

88. A method according to claim 87, wherein the DNA prime comprises administration of a DNA vaccine vector comprising a nucleic acid molecule according to claim 29, and the MVA boost comprises administration of an MVA vector comprising a nucleic acid molecule according to claim 29, optionally wherein the nucleic acid molecule according to claim 29 of the DNA vaccine vector encodes the same amino acid sequence as the nucleic acid molecule according to claim 29 of the MVA vector.

89.-91. (canceled)

92. A method according to claim 84, wherein the coronavirus is a beta-coronavirus, optionally wherein the β-coronavirus is a lineage B beta-coronarvirus.

93.-94. (canceled)

95. A method according to claim 92, wherein the lineage B β-coronavirus is SARS-CoV or SARS-CoV-2, preferably wherein the beta-coronavirus is a SARS-CoV-2 VOC, optionally wherein the beta-coronavirus is a SARS-CoV-2 beta, gamma, delta, or omicron VOC.

96.-103. (canceled)
